(19) United States
(12) Patent Application Publication
(10) Pub. No.: US 2019/0151312 A1
(43) Pub. Date: May 23, 2019

(54) HEPARAN SULFATE BIOSYNTHESIS INHIBITORS FOR THE TREATMENT OF DISEASES

(71) Applicant: BioMarin Pharmaceutical Inc., Novato, CA (US)

(72) Inventors: Shripad Bhagwat, San Diego, CA (US); Bing Wang, Novado, CA (US); Gregory R. Luedtke, San Diego, CA (US); Mark Spyvee, Hampstead, NH (US)

(21) Appl. No.: 15/517,814

(22) PCT Filed: Oct. 8, 2015
(86) PCT No.: PCT/US15/54761

§ 371 Date: Apr. 7, 2017

Related U.S. Application Data
(60) Provisional application No. 62/062,036, filed on Oct. 9, 2014.

(51) Int. Cl. A61K 31/506; A61P 25/28; A61P 25/16; A61P 37/06

(52) U.S. Cl. CPC A61K 31/506; A61P 25/28; A61K 38/1709; A61P 37/06; A61P 25/16

(57) ABSTRACT

Described herein are compounds of Formula I, methods of making such compounds, pharmaceutical compositions and medicaments containing such compounds, and methods of using such compounds to treat or prevent diseases or conditions in need of inhibition of heparan sulfate biosynthesis.

### CROSS REFERENCE TO RELATED APPLICATIONS

This application claims the benefit of priority under 35 U.S.C. § 119(e) to provisional U.S. Patent Application No. 62/062,036 filed Oct. 9, 2014, the entire contents of which are hereby incorporated by reference.

### FIELD

Described herein are compounds, methods of making the compounds, pharmaceutical compositions and medicaments containing the compounds, and methods of using the compounds to treat or prevent diseases or conditions in need of heparan sulfate biosynthesis inhibition.

### BACKGROUND

In lysosomal storage diseases, a person is missing a key enzyme (or has an enzyme which does not function normally) where the degradation of glycosylaminoglycans (GAGs) is impaired resulting in abnormal accumulation of these GAGs and disease. Substrate reduction therapy (SRT) offers an approach to treating diseases by inhibiting the formation of the substrate upon which the missing or abnormally-functioning enzyme acts. The aim is to reduce the rate of biosynthesis of the GAG to offset the catabolic defect, restoring the balance between the rate of biosynthesis and the rate of catabolism. Small molecule inhibitors of GAG biosynthesis can reduce the amount of substrate formed and, if able to cross the blood-brain barrier, have the potential to treat diseases with CNS pathology.

Heparan sulfate (HS) is one such GAG found in mammals comprising glucosamine and uronic acid groups. In certain instances, heparan sulfate is bound to a core protein via a linkage tetrasaccharide, which generally has the structure -GlcAβ3Galβ3Galβ4Xylβ-O—. The cell surface of most mammalian cells contains membrane anchored heparan sulfate proteoglycans (HSPGs) which have important functions in cell adhesion processes (Biochimica et Biphysica Acta-Molecular Cell Research 2001, 1541(3), 135). In certain lysosomal storage disorders, the ability of the lysosome to degrade and turnover HS is impaired.

Diseases associated with abnormal heparan sulfate accumulation include lysosomal storage disorders, such as mucopolysaccharidosis (MPS) I, II, IIIA, IIIB, IIIC, IIID, and VII disorders. These MPS disorders are caused by the inability to produce specific enzymes, which in turn leads to an abnormal storage of mucopolysaccharides, including heparan sulfate. (See Lawrence et al., Nat Chem Riot 2012, 8(2), 197 which is incorporated herein by reference in its entirety.) MPS I, II, IIIA, IIIB, IIIC, IIID, and VII disorders include Hurler syndrome (MPS I H), Scheie syndrome (MPS I S), Hurler-Scheie syndrome (MPS I H-S), Hunter syndrome (MPS II), Sanfilippo syndrome (e.g. Sanfilippo A (MPS III A), Sanfilippo B (MPS III B), Sanfilippo C (MPS III C), and Sanfilippo D (MPS III D)), and Sly syndrome (MPS VII).

HSPGs have been shown to promote formation of amyloid structures typical in amyloid diseases (such as Alzheimer's disease, Parkinson's disease, type 2 diabetes, and chronic hemodialysis-related amyloid) including colocalization with the amyloid plaques and may impart stability to the amyloid fibrils (J. Biol. Chem. 2002, 277, 18421; J. Neuroscience 2011, 31(5), 1644; PNAS 2005, 102(18), 6473).

HSPGs bound to the external surface of plasma membranes play an important role in the control of cell division and growth regulation, but are also implicated in brain pattering, synapse formation, axon regeneration and guidance, and are found in dense networks in active multiple sclerosis (MS) plaques (Hum. Mol. Genet. 2009, 18(4), 767) and thus reducing the amount of HSPGs can be useful in treating MS.

The compounds disclosed herein are useful for inhibiting heparan sulfate biosynthesis and associated diseases.

### SUMMARY OF THE INVENTION

In one aspect, provided is a compound of Formula I:

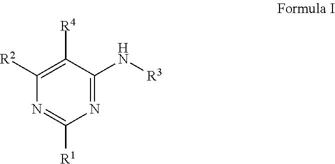

where
R¹ is heterocycloalkyl optionally substituted with 1, 2, 3, or 4 alkyl;
R² is a 9-membered bicyclic ring comprising 1, 2, 3, or 4 nitrogen atoms where one or both of the rings is aromatic, where a carbon atom in R² is the point of attachment to the pyrimidinyl in Formula I, and where R² is optionally substituted with 1 oxo and additionally optionally substituted with 1, 2, or 3 R^{2a} groups;
each R²a is independently selected from cyano, nitro, halo, hydroxy, alkyl, alkenyl, carboxy, alkoxycarbonyl, alkylcarbonyl, alkylsulfinyl, alkylsulfonyl, alkylsulfonyloxyalkyl, alkoxyalkyl, alkoxyalkenyl, aminoalkyl, alkylaminoalkyl, dialkylaminoalkyl, hydroxyalkyl, cycloalkyl, cycloalkylalkyl, heterocycloalkyl, heterocycloalkylalkyl, 1,3-dioxo-isoindolinylalkyl, —NR^{2b}R²′, and —OR^{2d}; where each heterocycloalkyl, either alone or as part of another group, is optionally substituted with 1, 2, 3, or 4 groups independently selected from alkyl, hydroxy, alkylcarbonyl, and alkoxycarbonyl;
R^{2b} is hydrogen or alkyl;
R^{2c} is hydrogen, alkyl, aminoalkyl, alkylaminoalkyl, dialkylaminoalkyl, alkoxyalkyl, hydroxyalkyl, alkylcarbonyl, cycloalkylcarbonyl, alkylsulfonyl, alkoxycarbonyl, cycloalkylalkyl, or heterocycloalkylalkyl; where the heterocycloalkyl in heterocycloalkylalkyl is optionally substituted with 1 or 2 groups independently selected from alkyl, hydroxy, alkylcarbonyl, and alkoxycarbonyl;
R^{2d} is alkyl, haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl; where each heterocycloalkyl, either alone or as part of another group, is optionally substituted with 1 or 2 groups independently selected from alkyl, hydroxy, alkylcarbonyl, and alkoxycarbonyl;
R³ is phenyl or heteroaryl each of which is optionally substituted with 1, 2, or 3 R^{3a} groups;
each R^{3a} is independently selected from —C(═NH)NHOH, cyano, nitro, halo, hydroxy, alkyl, alkoxycarbonyl, alkoxyalkyl, cycloalkyl, cycloalkylalkyl, heterocycloalkyl, heterocycloalkylalkyl, alkylcarbonyl, alkylsulfinyl, alkylsulfonyl, —OR^{3d}, —NR^{3b}R^{3c}, —C(O)NR^{3b}R^{3c}, —S(O)₂NR^{3b}R^{3c}, and heteroaryl optionally substituted with 1, 2, or 3 R⁵ groups; where the heterocycloalkyl either alone or as part of heterocycloalkylalkyl is optionally substituted with 1 or two alkyl groups;
R^{3b} is hydrogen or alkyl;
R^{3c} is hydrogen, alkyl, haloalkyl, aminoalkyl, alkylaminoalkyl, dialkylaminoalkyl, alkoxyalkyl, alkylcarbonyl, alkylsulfonyl, alkoxycarbonyl, cycloalkylalkyl, heterocycloalkyl, heterocycloalkylalkyl, or cycloalkyl optionally substituted with 1 or 2 alkyl; or R^{3b} and R^{3c} together with the nitrogen to which they are attached form heterocycloalkyl;
R^{ad} is alkyl, haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl;
R⁴ is hydrogen, methyl, halo, or —CN; and
each R⁵ is independently halo, hydroxy, alkoxycarbonyl, alkyl, haloalkyl, cycloalkyl, cycloalkylalkyl, heterocycloalkyl, heterocycloalkylalkyl, phenyl, or phenylmethyl which is optionally substituted with 1 or 2 alkoxy;
optionally a tautomer, a single stereoisomer or mixture of stereoisomers thereof and additionally optionally a pharmaceutically acceptable salt thereof;
   provided that
a) the compound is not N-(6-(1H-imidazo[4,5-b]pyridin-6-yl)-2-morpholinopyrimidin-4-yl)quinolin-3-amine;
b) when R³ is pyrazolyl substituted with one R^{3a}, then R^{3c} is not cyclopropyl; and
c) when R³ is phenyl substituted with one R^{3a}, then the one R^{3a} is not 3-7-membered cycloalkyl ring.

In certain embodiments, it is provided that:
a) the compound is not N-(6-(1H-imidazo[4,5-b]pyridin-6-yl)-2-morpholinopyrimidin-4-yl)quinolin-3-amine or N-(6-(1H-pyrazolo[3,4-b]pyridin-5-yl)-2-morpholinopyrimidin-4-yl)quinolin-3-amine;
b) when R³ is pyrazolyl substituted with one R^{3a}, then R^{3c} is not cyclopropyl; and
c) when R³ is phenyl substituted with one R^{3ai}, then the one R^{3a i}is not 3-7-membered cycloalkyl ring.

Embodiments can include any one or more of the following features.

R¹ can be dihydro-2H-pyran-4-yl, tetrahydro-2H-pyran-4-yl, or morpholin-4-yl, each of which is optionally substituted with 1 or 2 alkyl; R³ can be phenyl substituted with 1, 2, or 3 R^{3c} groups independently selected from —C(═NH)NHOH, cyano, nitro, halo, alkyl, alkoxycarbonyl, alkoxyalkyl, cycloalkyl, cycloalkylalkyl, heterocycloalkyl, heterocycloalkylalkyl, alkylcarbonyl, alkylsulfinyl, alkylsulfonyl, —OR^{3d}, —NR^{3b}R^{3c}, —C(O)NR^{3b}R^{3c}, —S(O)₂NR^{3b}R^{3c}, and heteroaryl optionally substituted with 1, 2, or 3 R⁵ groups; where the heterocycloalkyl either alone or as part of heterocycloalkylalkyl can be optionally substituted with 1 alkyl; provided that R³ is not 3-amino-phenyl or 3,4-dimethylphenyl; or R³ can be 6-10 membered heteroaryl each of which is substituted with 1, 2, or 3 R^{3c} groups independently selected from —C(═NH)NHOH, cyano, nitro, halo, hydroxy, alkyl, alkoxycarbonyl, alkoxyalkyl, cycloalkyl, cycloalkylalkyl, heterocycloalkyl, heterocycloalkylalkyl, alkylcarbonyl, alkylsulfinyl, alkylsulfonyl, —OR^{3d}, —NR^{3b}R^{3a}, —C(O)NR^{3b}R^{3a}, —S(O)₂NR^{3b}R^{3c}, and heteroaryl optionally substituted with 1, 2, or 3 R⁵ groups; provided that R³ is not 2-oxo-1H-benzo[d]imidazolyl, 1-ethyl-2-methyl-1H-benzo[d]imidazolyl, or 1-acetyl-indolinyl; R^{3b} can be hydrogen or alkyl; R^{3c} can be hydrogen, alkyl, haloalkyl, aminoalkyl, alkylaminoalkyl, dialkylaminoalkyl, alkoxyalkyl, alkylcarbonyl, alkylsulfonyl, alkoxycarbonyl, cycloalkylalkyl, heterocycloalkyl, heterocycloalkylalkyl, or cycloalkyl optionally substituted with 1 or 2 alkyl; or R^{3b} and R^{3c} together with the nitrogen to which they are attached can form heterocycloalkyl; R^{3d} is haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl;
R⁴ can be hydrogen, methyl, or halo; and each R⁵ can be independently halo, hydroxy, alkoxycarbonyl, alkyl, haloalkyl, cycloalkyl, cycloalkylalkyl, heterocycloalkyl, heterocycloalkylalkyl, phenyl, or phenylmethyl which is optionally substituted with 1 or 2 alkoxy; optionally as a single stereoisomer or mixture of stereoisomers thereof and additionally optionally as a pharmaceutically acceptable salt thereof.

R¹ can be morpholin-4-yl; R² can be a 9-membered bicyclic ring comprising 1, 2, or 3 nitrogen atoms where one or both of the rings is aromatic, where a carbon atom in R² is the point of attachment to the pyrimidinyl in Formula I, and where R² is optionally substituted with 1 oxo and additionally substituted with 1, 2, or 3 R^{2a} groups; each R^{2a} can be independently selected from halo, alkyl, alkoxyalkyl, aminoalkyl, alkylaminoalkyl, dialkylaminoalkyl, hydroxyalkyl, cycloalkyl, cycloalkylalkyl, heterocycloalkyl, heterocycloalkylalkyl, 1,3-dioxo-isoindolinylalkyl, and —NR^{2b}R^{2c}; where each heterocycloalkyl, either alone or as part of another group, is optionally substituted with alkyl; R^{2b} can be hydrogen or alkyl; R^{2c} can be alkyl, aminoalkyl, alkylaminoalkyl, dialkylaminoalkyl, alkoxyalkyl, or heterocycloalkylalkyl; R³ can be phenyl or heteroaryl each of which is optionally substituted with 1, 2, or 3 R^{3a} groups; each R^{3a} can be independently selected from cyano, halo, alkyl, alkoxycarbonyl, cycloalkyl, alkylcarbonyl, alkylsulfinyl, alkylsulfonyl, —OR^{3d}, —NR^{3b}R^{3a}, —C(O)NR^{3b}R^{3a}, —S(O)₂NR^{3b}R^{3a}, and heteroaryl optionally substituted with R⁵; R^{3b} can be hydrogen or alkyl; R^{3c} can be hydrogen, alkyl, alkylcarbonyl, alkylsulfonyl, cycloalkylalkyl, or cycloalkyl optionally substituted with alkyl; R^{3d} can be alkyl; R⁴ can be hydrogen, or halo; and each R⁵ can be independently halo, alkyl, haloalkyl, cycloalkyl, or phenylmethyl which is optionally substituted with alkoxy; optionally as a single stereoisomer or mixture of stereoisomers thereof and additionally optionally as a pharmaceutically acceptable salt thereof.

R¹ can be morpholin-4-yl.

R² can be indazolyl or pyrazolopyridinyl, each of which is optionally substituted on any atom of the ring with 1, 2, or 3 R^{2a} groups. R² can be benzimidazolyl or imidazopyridinyl, each of which is optionally substituted on any atom of the ring with 1, 2, or 3 R^{2a} groups. R² can be 2-oxo-1H-benzo[d]imidazolyl optionally substituted on any atom of the ring with 1, 2, or 3 R^{2a} groups. R² can be indazolyl or benzimidazolyl, each of which is optionally substituted on any atom of the ring with 1, 2, or 3 R^{2a} groups.

R² can be substituted with 1, 2, or 3 R^{2a} groups independently selected from alkyl, hydroxyalkyl, alkoxyalkyl, halo, cycloalkyl, cycloalkylalkyl, heterocycloalkyl, heterocycloalkylalkyl optionally substituted with one alkyl, aminoalkyl, alkylaminoalkyl, dialkylaminoalkyl, 1,3-dioxo-isoindolinylalkyl, and —NR^{2b}R^{2c}. In certain embodiments, R^{2a} is alkyl or heterocycloalkylalkyl. In other embodiments, R^{2a} is C₁₋₃ alkyl or heterocycloalkyl(C₁₋₃)alkyl, where the heterocycloalkyl group is morpholinyl, piperzinyl, or pyrrolodinyl.

R³ can be phenyl substituted with one or two R^{3a} groups. For example, R³ can be phenyl substituted with halo (e.g., chloro). As another example, R³ can be phenyl substituted with —C(O)NR^{3b}R^{3c}. As a further example, R³ can be phenyl substituted with a 5-membered heteroaryl optionally substituted with one R⁵. In still yet another example, R³ can be phenyl substituted with R^{3c} where R^{3c} is triazolyl, oxazolyl, imidazolyl, oxadiazolyl, pyrazolyl, or pyrrolyl, each of which is optionally substituted with one R⁵; or where.g., R^{3c} is triazolyl, oxazolyl, imidazolyl, oxadiazolyl, pyrazolyl, or pyrrolyl, each of which is optionally substituted with alkyl, halo, haloalkyl, cycloalkyl, or phenylmethyl, where the phenylmethyl is optionally substituted with alkoxy.

R³ can be a 6-10 membered heteroaryl substituted with 1, 2, or 3 R^{3a} groups. For example, R³ can be pyridyl substituted with 1, 2, or 3 R^{3c} groups. As another example, R³ can be a 9-membered heteroaryl with 1, 2, or 3 nitrogen atoms, optionally substituted with 1, 2, or 3 R^{3a} groups; e.g., R³ can be indolyl, benzoisoxazolyl, indazolyl, benzotriazolyl, benzoxazolyl, or benzimidazolyl, each of which is optionally substituted with 1, 2, or 3 alkyl groups.

R¹ can be morpholin-4-yl; R² can be indazolyl or benzimidazolyl, either of which is substituted with at least one R^{2a} independently selected from alkyl, hydroxyalkyl, alkoxyalkyl, halo, cycloalkyl, cycloalkylalkyl, heterocycloalkyl, heterocycloalkylalkyl optionally substituted with one alkyl, aminoalkyl, alkylaminoalkyl, dialkylaminoalkyl, 1,3-dioxo-isoindolinylalkyl, and —NR^{2b}R^{2c}; R³ can be phenyl substituted with at least one R^{3a} independently selected from halo, —C(O)NR^{3b}R^{3c}, and a 5-membered heteroaryl optionally substituted with one R⁵; or R³ can be pyridinyl, indolyl, benzoisoxazolyl, indazolyl, benzotriazolyl, benzoxazolyl, or benzimidazolyl, each of which is optionally substituted with 1, 2, or 3 alkyl groups.

The compounds can have Formula I(a) as defined anywhere herein, optionally as a single stereoisomer or mixture of stereoisomers thereof and additionally optionally as a pharmaceutically acceptable salt thereof.

R¹ can be morpholin-4-yl; R² can be indazolyl or benzimidazolyl, either of which is substituted with at least one R^{2a} independently selected from alkyl, hydroxyalkyl, alkoxyalkyl, halo, cycloalkyl, cycloalkylalkyl, heterocycloalkyl, heterocycloalkylalkyl optionally substituted with 1 alkyl, aminoalkyl, alkylaminoalkyl, dialkylaminoalkyl, 1,3-dioxo-isoindolinylalkyl, and —NR^{2b}R^{2c}; R³ can be phenyl substituted with at least one R^{3c} independently selected from halo, —C(O)NR^{3b}R^{3c}, and a 5-membered heteroaryl optionally substituted with one R⁵; or R³ can be pyridinyl, indolyl, benzoisoxazolyl, indazolyl, benzotriazolyl, benzoxazolyl, or benzimidazolyl, each of which is optionally substituted with 1, 2, or 3 alkyl groups.

In another aspect, compounds having Formula II are featured:

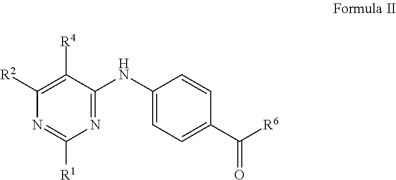

wherein:
R¹ is heterocycloalkyl optionally substituted with 1, 2, 3, or 4 alkyl;
R² is a 9-membered bicyclic ring comprising 1, 2, 3, or 4 nitrogen atoms where one or both of the rings is aromatic, where a carbon atom in R² is the point of attachment to the pyrimidinyl in Formula II, and where R² is optionally substituted with 1 oxo and additionally optionally substituted with 1, 2, or 3 R^{2a} groups;
each R²a is independently selected from cyano, nitro, halo, hydroxy, alkyl, alkenyl, carboxy, alkoxycarbonyl, alkylcarbonyl, alkylsulfinyl, alkylsulfonyl, alkylsulfonyloxyalkyl, alkoxyalkyl, alkoxyalkenyl, aminoalkyl, alkylaminoalkyl, dialkylaminoalkyl, hydroxyalkyl, cycloalkyl, cycloalkylalkyl, heterocycloalkyl, heterocycloalkylalkyl, 1,3-dioxo-isoindolinylalkyl, —NR^{2b}R^{2c}, and —OR^{2d}; where each heterocycloalkyl, either alone or as part of another group, is optionally substituted with 1, 2, 3, or 4 groups independently selected from alkyl, hydroxy, alkylcarbonyl, and alkoxycarbonyl;
R^{2b} is hydrogen or alkyl;
R^{2c} is hydrogen, alkyl, aminoalkyl, alkylaminoalkyl, dialkylaminoalkyl, alkoxyalkyl, hydroxyalkyl, alkylcarbonyl, cycloalkylcarbonyl, alkylsulfonyl, alkoxycarbonyl, cycloalkylalkyl, or heterocycloalkylalkyl; where the heterocycloalkyl in heterocycloalkylalkyl is optionally substituted with 1 or 2 groups independently selected from alkyl, hydroxy, alkylcarbonyl, and alkoxycarbonyl;
R^{2d} is alkyl, haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl; where each heterocycloalkyl, either alone or as part of another group, is optionally substituted with 1 or 2 groups independently selected from alkyl, hydroxy, alkylcarbonyl, and alkoxycarbonyl;
R⁴ is hydrogen, methyl, halo, or —CN; and
R⁶ is halo, hydroxy, or alkoxy; optionally as a single stereoisomer or mixture of stereoisomers thereof and additionally optionally as a pharmaceutically acceptable salt thereof.

In a further aspect, provided is a pharmaceutical composition comprising 1) a Compound of Formula I optionally as a tautomer, a single stereoisomer or mixture of stereoisomers thereof and additionally optionally as a pharmaceutically acceptable salt thereof, and 2) a pharmaceutically acceptable excipient.

In a further aspect, provided is a method of treating a disease or disorder comprising administering to a subject in need thereof a Compound of Formula I, optionally as a tautomer, a single stereoisomer or mixture of stereoisomers thereof and additionally optionally as a pharmaceutically acceptable salt thereof, or the pharmaceutical composition thereof additionally comprising a pharmaceutically acceptable excipient or pharmaceutically acceptable carrier. Methods of treatment are also provided, which include administering to a subject having any one or more of the diseases or disorders described herein a Compound of Formula I, optionally as a tautomer, a single stereoisomer or mixture of stereoisomers thereof and additionally optionally as a pharmaceutically acceptable salt thereof, or the pharmaceutical composition thereof additionally comprising a pharmaceutically acceptable excipient or pharmaceutically acceptable carrier. Any of the methods described herein can further include identifying the subject (e.g., a subject in need of treatment for one or more of the diseases and disorders described herein).

In a further aspect, it is provided a method of making a compound of Formula I, comprising
a) treating an intermediate of formula 102

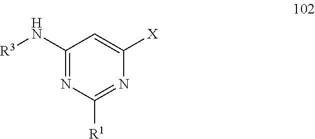

or a salt thereof where X is halo, where R¹, R³, and all other groups are as defined in the Summary of the Invention or as in any of the embodiments described herein; with an intermediate of formula R²B(OR)₂ (where each R is hydrogen or alkyl or together with the atoms to which they are attached form a carbocyclic ring and where R² is as defined in the Summary of the Invention or as in any of the embodiments described herein) in the presence of a catalyst and a base to yield a Compound of Formula I; or
b) treating an intermediate of formula 101

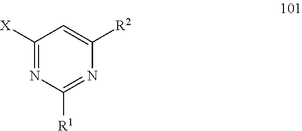

or a salt thereof where X is halo, where R¹, R², and all other groups are as defined in the Summary of the Invention or as in any of the embodiments described herein; with an intermediate of formula R³NH₂ (where R³ is as defined in the Summary of the Invention or as in any of the embodiments described herein) in the presence of a catalyst and a base to yield a Compound of Formula I; and
c) optionally separating individual isomers.

### DETAILED DESCRIPTION

### Abbreviations

| Abbreviation | Meaning |
|---|---|
| ACN | acetonitrile |
| aq | aqueous |
| BINAP | (2,2′-bis(diphenylphosphino)-1,1′-binaphthyl) |
| Boc | tert-butoxycarbonyl |
| conc | concentrated |
| dba | dibenzylideneacetone |
| DBU | 1,8-Diazabicyclo[5.4.0]undec-7-ene |
| DIPEA | diisoproylethylamine |
| DCM | dichloromethane |
| DMA | dimethylacetamide |
| DME | 1,2-dimethoxy ethane |
| DMF | dimethylformamide |
| DMS | dimethylsulfate |
| DMSO | dimethyl sulfoxide |
| dppf | 1,1′-bis(diphenylphosphanyl) ferrocene |
| EDCI | 1-ethyl-3-(3-dimethylaminopropyl)carbodiimide |
| eq | equivalents |
| EtOAc | ethyl acetate |
| g | gram |
| h | hours |
| HATU | O-(7-azabenzotriazol-1-yl)-N,N,N′,N′-tetramethyluronium |
| | hexafluorophosphate |
| HOBt | hydroxybenzotriazole |
| HPLC | high performance liquid chromatography |
| IPA | isopropanol |
| LCMS | liquid chromatography mass spectrometry |
| mg | milligram |
| mHz | megahertz |
| min | minute |
| mL | milliliter |
| μL | microliter |
| Ms | mesyl |
| NBS | N-bromosuccinimide |
| NMM | N-methylmorpholine |
| NMP | N-methylpyrrolidone |
| NMR | nuclear magnetic resonance |
| PMB | p-methoxybenzyl |
| PYBOP | (benzotriazol-1-yl-oxytripyrrolidinophosphonium |
| | hexafluorophosphate) |
| rt or RT | room temperature |
| sat | saturated |
| TEA | triethylamine |
| TFA | trifluoroacetic acid |
| THF | tetrahydrofuran |
| TLC | thin layer chromatography |
| xantphos | 4,5-Bis(diphenylphosphino)-9,9-dimethylxanthene |

### Definitions

To facilitate understanding of the disclosure set forth herein, a number of terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

As used throughout this application and the appended claims, the following terms have the following meanings:

“About” preceding a numerical value refers to a range of values ±10% of the value specified.

“Acceptable” with respect to a formulation, composition or ingredient, as used herein, means having no persistent detrimental effect on the general health of the subject being treated.

“Alkenyl” means a straight or branched hydrocarbon radical having from 2 to 8 carbon atoms and at least one double bond and includes ethenyl, propenyl, 1-but-3-enyl, 1-pent-3-enyl, 1-hex-5-enyl and the like. “Lower alkenyl” means an alkenyl group having one to six carbon atoms.

“Alkoxy” means an —OR group where R is alkyl, as defined herein. Illustrative examples include, but are not limited to, methoxy, ethoxy, propoxy, 2-propoxy, butoxy, tert-butoxy, pentyloxy, and hexyloxy.

“Alkoxyalkenyl” means an alkenyl group, as defined herein, substituted with at least one, in another example 1, 2, or 3 alkoxy group(s), as defined herein.

“Alkoxyalkyl” means an alkyl group, as defined herein, substituted with at least one, in another example 1, 2, or 3 alkoxy group(s), as defined herein. When “C₁-C₆” is used before “alkoxyalkyl,” the “C₁-C₆” modifies both the alkyl in the alkoxy portion as well as the alkyl portion.

“Alkoxycarbonyl” means a —C(O)R group where R is alkoxy, as defined herein.

“Alkyl” means a straight or branched saturated hydrocarbon radical containing from 1-10 carbon atoms, in another example 1-6 carbon atoms. Illustrative examples include, but are not limited to, methyl, ethyl, n-propyl, iso-propyl, n-butyl, sec-butyl, tert-butyl, n-pentyl, isopentyl, neopentyl, n-hexyl, 3-methylhexyl, 2,2-dimethylpentyl, 2,3-dimethylhexyl, n-heptyl, n-octyl, n-nonyl, and n-decyl.

“Alkylamino” means a —NHR radical where R is alkyl as defined herein, or an N-oxide derivative thereof, e.g., methylamino, ethylamino, n-, iso-propylamino, n-, iso-, tert-butylamino, or methylamino-N-oxide, and the like.

“Alkylaminoalkyl” means an alkyl group substituted with at least one, in another example 1, 2, or 3 alkylamino groups, as defined herein. When “C₁-C₆” is used before “alkylaminoalkyl,” the “C₁-C₆” modifies both of the alkyl portions.

“Alkylcarbonyl” means a —C(O)R group where R is alkyl, as defined herein.

“Alkylsulfinyl” means an —S(O)R group where R is alkyl, as defined herein.

“Alkylsulfonyl” means an —S(O)₂R group where R is alkyl as defined herein.

“Alkylsulfonyloxyalkyl” means an alkyl group substituted with 1 or 2 -OS(O)2alkyl group(s), e.g. —CH₂CH₂OS(O)₂CH₃.

“Amino” means an —NH₂ group.

“Aminoalkyl” means an alkyl group substituted with at least one, for example one, two, or three, amino groups.

“Carboxy” means a —C(O)OH group.

“Cycloalkyl” means a monocyclic or fused bicyclic, saturated or partially unsaturated (but not aromatic), hydrocarbon radical of three to ten carbon ring atoms. Fused bicyclic hydrocarbon radical includes bridged rings. Cycloalkyl includes spirocycloalkyl rings. Unless stated otherwise, the valency of the group may be located on any atom of any ring within the radical, valency rules permitting. One or two ring carbon atoms may be replaced by a —C(O)—, —C(S)—, or —C(═NH)— group.

In one embodiment, cycloalkyl includes but is not limited to:

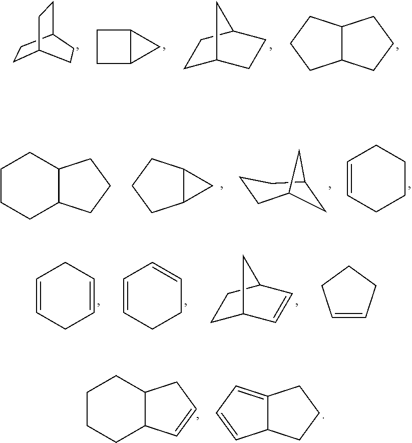

“Cycloalkylalkyl” means an alkyl group, as defined herein, substituted with at least one, in another example 1 or 2, cycloalkyl groups as defined herein.

“Cycloalkylcarbonyl” means a —C(O)R group where R is cycloalkyl, as defined herein.

“Dialkylamino” means an —NRR′ radical where R and R′ are independently alkyl as defined herein, or an N-oxide derivative, or a protected derivative thereof, e.g., dimethylamino, diethylamino, N,N-methylpropylamino or N,N-methylethylamino, and the like.

“Dialkylaminoalkyl” means an alkyl group substituted with at least one, in another example 1, 2, or 3 dialkylamino groups, as defined herein. When “C₁-C₆” is used before “dialkylaminoalkyl,” the “C₁-C₆” modifies all of the alkyl portions.

“Halo” means a halogen, or fluoro, chloro, bromo, or iodo.

“Haloalkyl” means an alkyl group substituted with one or more halo atoms, in another example by 1, 2, 3, 4, 5, or 6 halo atoms, in another example by 1, 2, or 3 halo atoms. Examples include, but are not limited to, trifluoromethyl, chloromethyl, and the like.

“Heteroaryl” means monocyclic, fused bicyclic, or fused tricyclic, radical of 5 to 14 ring atoms containing one or more, in another example one, two, three, or four ring heteroatoms independently selected from —O—, —S(O)ₙ— (n is 0, 1, or 2), —N—, —N(H)—, and N-oxide, and the remaining ring atoms being carbon, wherein the ring comprising a monocyclic radical is aromatic and wherein at least one of the fused rings comprising a bicyclic or tricyclic radical is aromatic (but does not have to be a ring which contains a heteroatom, e.g. 2,3-dihydrobenzo[b][1,4]dioxin-6-yl). One or two ring carbon atoms of any nonaromatic rings comprising a bicyclic or tricyclic radical may be replaced by a —C(O)—, —C(S)—, or —C(═NH)— group. Fused bicyclic radical includes bridged ring systems. Unless stated otherwise, the valency may be located on any atom of any ring of the heteroaryl group, valency rules permitting.

In one embodiment, heteroaryl includes, but is not limited to, triazolyl, tetrazolyl, pyrrolyl, imidazolyl, thienyl, furanyl, pyrazolyl, oxazolyl, isooxazolyl, oxadiazolyl, thiadiazolyl, indolyl, 2,3-dihydro-1H-indolyl (including, for example, 2,3-dihydro-1H-indol-2-yl or 2,3-dihydro-1H-indol-5-yl, and the like), indazolyl, phthalimidyl, benzimidazolyl, benzoxazolyl, benzofuranyl, benzothienyl, benzopyranyl, benzothiazolyl, pyridinyl, pyrazinyl, pyrimidinyl, pyridazinyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), pyrrolo[1,2-b]pyridazinyl, imidazo[1,2-a]pyridinyl, thiazolyl, benzo[d][1,3]dioxolyl, 2,3-dihydrobenzo[b][1,4]dioxinyl, furo[2,3-d]thiazolyl, thieno[2,3-d]oxazolyl, thieno[3,2-b]furanyl, furo[2,3-d]pyrimidinyl, furo[3,2-b]pyridinyl, furo[3,2-c]pyridinyl, 6,7-dihydro-5H-cyclopenta[b]pyridinyl, and 7,8-dihydro-6H-cyclopenta[g]quinoxalinyl; and derivatives, N-oxide and protected derivatives thereof.

“Heterocycloalkyl” means a saturated or partially unsaturated (but not aromatic) monovalent monocyclic group of 3 to 9 ring atoms or a saturated or partially unsaturated (but not aromatic) monovalent fused bicyclic group of 5 to 12 ring atoms in which one or more heteroatoms, for example one, two, three, or four ring heteroatoms, independently selected from —O—, —S(O)ₙ— (n is 0, 1, or 2), —N═, —NH—, and N-oxide, the remaining ring atoms being carbon. One or two ring carbon atoms may be replaced by a —C(O)—, —C(S)—, or —C(═NH)— group. Fused bicyclic radical includes bridged ring systems. Heterocycloalkyl groups include spiro heterocycloalkyl rings. Unless otherwise stated, the valency of the group may be located on any atom of any ring within the radical, valency rules permitting.

In one embodiment, heterocycloalkyl includes, but is not limited to, azetidinyl, pyrrolidinyl, 2-oxopyrrolidinyl, 2,5-dihydro-1H-pyrrolinyl, 2,5-dioxo-1H-pyrrolyl, 2,5-dioxo-pyrrolidinyl, 2,5-dihydro-1H-pyrrolyl, piperidinyl, 2-oxopiperidinyl, 4-piperidonyl, morpholinyl, piperazinyl, 2-oxopiperazinyl, dioxopiperazinyl, pyranyl, tetrahydropyranyl, tetrahydrothiopyranyl, 1,3-dioxinyl, 1,3-dioxanyl, 1,4-dioxinyl, 1,4-dioxanyl, thiomorpholinyl, thiamorpholinyl, perhydroazepinyl, pyrazolidinyl, imidazolinyl, imidazolidinyl, 2,4-dioxo-imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydroindolyl, octahydroisoindolyl, decahydroisoquinolyl, tetrahydrofuryl, 2-oxa-6-azaspiro[3.4]octanyl, 6-azaspiro[2.5]octanyl, 2,6-diazaspiro[3.3]heptanyl, 2-azaspiro[3.3]heptanyl, 7-azabicyclo[2.2.1]heptanyl, and 8-azabicyclo[3.2.1]octanyl, and the derivatives thereof and N-oxide (for example 1-oxido-pyrrolidin-1-yl) or a protected derivative thereof.

“Heterocycloalkylalkyl” means an alkyl group substituted with at least one, in another example 1 or 2, heterocycloalkyl groups, as defined herein.

“Hydroxyalkyl” means an alkyl group, as defined herein substituted with at least one, in another example 1, 2, or 3 hydroxy groups.

“Stereoisomers” include (but are not limited to) geometric isomers, enantiomers, diastereomers, and mixtures of geometric isomers, enantiomers or diastereomers. In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic column.

As used herein, “amelioration” of the symptoms of a particular disorder by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The terms “effective amount” or “therapeutically effective amount,” as used herein, refer to a sufficient amount of an agent or a compound being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

“Excipient” or “pharmaceutically acceptable excipient” means a pharmaceutically-acceptable material, composition, or vehicle, such as a liquid or solid filler, diluent, solvent, or encapsulating material. The term excipient includes carriers and diluents. The term “carrier” includes pharmaceutically inert, inorganic or organic carriers for the production of pharmaceutical compositions. Lactose, corn starch, or derivatives thereof, talc, stearic acid or its salts and the like can be used, for example, as such carriers for tablets, coated tablets, dragees and hard gelatin capsules. Suitable carriers for soft gelatin capsules are, for example, vegetable oils, waxes, fats, semi-solid and liquid polyols and the like. Depending on the nature of the active ingredient no carriers are, however, usually required in the case of soft gelatin capsules, other than the soft gelatin itself. Suitable carriers for the production of solutions and syrups are, for example, water, polyols, glycerol, vegetable oils and the like. Suitable carriers for suppositories are, for example, natural or hardened oils, waxes, fats, semi-liquid or liquid polyols and the like. The term “diluent” refers to chemical compounds that are used to dilute the compound of interest prior to delivery. Diluents include chemicals used to stabilize compounds because they provide a more stable environment. Salts dissolved in buffered solutions (which also can provide pH control or maintenance) are utilized as diluents In certain embodiments, including, but not limited to a phosphate buffered saline solution. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

“Pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt is not specifically limited as far as it can be used in medicaments. Examples of a salt that the compound of the present invention forms with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

“Subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

“Treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

### Embodiments

The following paragraphs present a number of embodiments of the compounds disclosed herein. In each instance the embodiment includes both the recited compound(s) as well as a single stereoisomer or mixture of stereoisomers thereof, as well as a pharmaceutically acceptable salt thereof. The compounds include the N-oxides or pharmaceutically acceptable salts thereof. In some situations, the compounds exist as tautomers. All tautomers are included within the scope of the compounds presented herein.

Excluded from any and all embodiments is the compound N-(6-(1H-imidazo[4,5-b]pyridin-6-yl)-2-morpholinopyrimidin-4-yl)quinolin-3-amine. Excluded from any and all embodiments is the compound N-(6-(1H-pyrazolo[3,4-b]pyridin-5-yl)-2-morpholinopyrimidin-4-yl)quinolin-3-amine. Excluded from any and all embodiments is the compound where R³ is pyrazolyl substituted with one R^{a} where the R^{3a} is cyclopropyl. Excluded from any and all embodiments is the compound where R³ is phenyl substituted with one R^{3a} where the 1 R^{3a} is a 3-7-membered cycloalkyl ring.

In certain embodiments, the compound of Formula I is that where:
R¹ is morpholinyl;
R² is a indazolyl, pyrazolopyridinyl, benzimidazolyl, imidazopyridinyl, or 2(3H)-oxo-benzoimidazolyl; where a carbon atom in R² is the point of attachment to the pyrimidinyl in Formula I, and where R² is optionally substituted with 1 or 2 R^{2a} groups;
each R^{2a} is independently selected from halo, alkyl, alkenyl, alkylsulfonyloxyalkyl, alkoxyalkyl, aminoalkyl, alkylaminoalkyl, dialkylaminoalkyl, hydroxyalkyl, cycloalkyl, cycloalkylalkyl, heterocycloalkyl, heterocycloalkylalkyl, 1,3-dioxo-isoindolinylalkyl, and —NR^{2b}R^{2c}; where each heterocycloalkyl, either alone or as part of another group, is optionally substituted with 1 alkyl;
R^{2b} is hydrogen or alkyl;
R^{2c} is hydrogen, alkyl, aminoalkyl, alkylaminoalkyl, dialkylaminoalkyl, alkoxyalkyl, or heterocycloalkylalkyl; where the heterocycloalkyl in heterocycloalkylalkyl is optionally substituted with 1 or 2 groups independently selected from alkyl, hydroxy, alkylcarbonyl, and alkoxycarbonyl;
R³ is phenyl, pyridinyl, or a bicyclic heteroaryl, each of which is optionally substituted with 1, 2, or 3 R^{3a} groups; p0 each R^{3a} is independently selected from —C(═NH)NHOH, cyano, halo, alkyl, alkylcarbonyl, alkoxycarbonyl, cycloalkyl, alkylsulfinyl, alkylsulfonyl, —OR^{3d}, —NR^{3b}R^{3a}, —C(O)NR^{3b}R^{3a}, —S(O)₂NR^{3b}R^{3c}, heterocycloalkyl, and heteroaryl optionally substituted with 1 R⁵ groups; where the heterocycloalkyl is optionally substituted with 1 alkyl;
R^{3b} is hydrogen or alkyl; R^{3c} is hydrogen, alkyl, alkylcarbonyl, alkylsulfonyl, alkoxycarbonyl, cycloalkylalkyl, or cycloalkyl optionally substituted with 1 alkyl;
R^{3d} is alkyl, methyl, or fluoro;
R⁴ is hydrogen or methyl; and
each R⁵ is independently halo, alkoxycarbonyl, alkyl, haloalkyl, cycloalkyl, or phenylmethyl which is optionally substituted with 1 alkoxy.

In certain embodiments, the compound of Formula I is that where:
R¹ is heterocycloalkyl optionally substituted with 1, 2, 3, or 4 alkyl;
R² is a 9-membered bicyclic ring comprising 1, 2, 3, or 4 nitrogen atoms where one or both of the rings is aromatic, where a carbon atom in R² is the point of attachment to the pyrimidinyl in Formula I, and where R² is optionally substituted with 1 oxo and additionally optionally substituted with 1, 2, or 3 R^{2a} groups;
each R^{2a} is independently selected from cyano, nitro, halo, hydroxy, alkyl, alkenyl, carboxy, alkoxycarbonyl, alkylcarbonyl, alkylsulfinyl, alkylsulfonyl, alkylsulfonyloxyalkyl, alkoxyalkyl, alkoxyalkenyl, aminoalkyl, alkylaminoalkyl, dialkylaminoalkyl, hydroxyalkyl, cycloalkyl, cycloalkylalkyl, heterocycloalkyl, heterocycloalkylalkyl, 1,3-dioxo-isoindolinylalkyl, —NR^{2b}R^{2c}, and —OR^{2d}; where each heterocycloalkyl, either alone or as part of another group, is optionally substituted with 1, 2, 3, or 4 groups independently selected from alkyl, hydroxy, alkylcarbonyl, and alkoxycarbonyl;
R^{2b} is hydrogen or alkyl;
R^{2c} is hydrogen, alkyl, aminoalkyl, alkylaminoalkyl, dialkylaminoalkyl, alkoxyalkyl, hydroxyalkyl, alkylcarbonyl, cycloalkylcarbonyl, alkylsulfonyl, cycloalkylalkyl, or heterocycloalkylalkyl; where the heterocycloalkyl in heterocycloalkylalkyl is optionally substituted with 1 or 2 groups independently selected from alkyl, hydroxy, alkylcarbonyl, and alkoxycarbonyl;
R^{2d} is alkyl, haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl; where each heterocycloalkyl, either alone or as part of another group, is optionally substituted with 1 or 2 groups independently selected from alkyl, hydroxy, alkylcarbonyl, and alkoxycarbonyl;
R³ is phenyl or heteroaryl with 5-9 ring atoms, each of which is optionally substituted with 1, 2, or 3 R^{3c} groups;
each R^{3c} is independently selected from —C(═NH)NHOH, cyano, nitro, halo, hydroxy, alkyl, alkoxycarbonyl, alkoxyalkyl, cycloalkylalkyl, heterocycloalkyl, heterocycloalkylalkyl, alkylcarbonyl, alkylsulfinyl, alkylsulfonyl, —OR^{3d}, —NR^{3b}R^{3c}, —C(O)NR^{3b}R^{3c}, —S(O)₂NR^{3b}R^{3c}, and heteroaryl optionally substituted with 1, 2, or 3 R⁵ groups; where the heterocycloalkyl either alone or as part of heterocycloalkylalkyl is optionally substituted with 1 or two alkyl groups;
R^{3b} is hydrogen or alkyl;
R^{3c} is hydrogen, alkyl, haloalkyl, aminoalkyl, alkylaminoalkyl, dialkylaminoalkyl, alkoxyalkyl, alkylcarbonyl, alkylsulfonyl, alkoxycarbonyl, cycloalkylalkyl, heterocycloalkyl, heterocycloalkylalkyl, or cycloalkyl optionally substituted with 1 or 2 alkyl; or R^{3b} and R^{3c} together with the nitrogen to which they are attached form heterocycloalkyl;
R^{3d} is alkyl, haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl;
R⁴ is hydrogen, methyl, halo, or —CN; and
each R⁵ is independently halo, hydroxy, alkoxycarbonyl, alkyl, haloalkyl, cycloalkyl, cycloalkylalkyl, heterocycloalkyl, heterocycloalkylalkyl, phenyl, or phenylmethyl which is optionally substituted with 1 or 2 alkoxy; optionally as a single stereoisomer or mixture of stereoisomers thereof and additionally optionally as a pharmaceutically acceptable salt thereof.

In certain embodiments of the compounds described herein, R¹ is dihydro-2H-pyran-4-yl, tetrahydro-2H-pyran-4-yl, or morpholin-4-yl, each of which is optionally substituted with 1, 2, 3, or 4 alkyl, or with 1 or 2 alkyl; and all other groups are as defined in the Summary of the Invention or in any of the embodiments. In certain embodiments, R¹ is morpholinyl optionally substituted with 1, 2, 3, or 4 alkyl or with 1 or 2 alkyl; and all other groups are as defined in the Summary of the Invention or in any of the embodiments. In certain embodiments, R¹ is morpholin-4-yl optionally substituted with 1, 2, 3, or 4 alkyl or with 1 or 2 alkyl; and all other groups are as defined in the Summary of the Invention or in any of the embodiments. In certain embodiments, R¹ is unsubstituted morpholin-4-yl; and all other groups are as defined in the Summary of the Invention or in any of the embodiments.

In certain embodiments of the compounds described herein, R² is

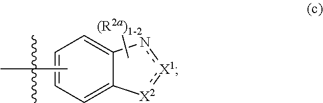

where one of the bonds in

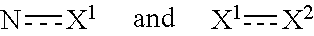

is a single bond and the other is a double bond, X¹ and X² are independently N, NR^{2a}, CH, or CR^{2a}; and all other groups are as defined in the Summary of the Invention or in any of the embodiments. In certain embodiments, R² is ring (c) and is attached by a carbon in the 5- or 6-position of ring (c) to the pyrimidinyl; and all other groups are as defined in the Summary of the Invention or in any of the embodiments. In certain embodiments, R² is ring (c); R^{2a} is alkyl, cycloalkyl, halo, or —NR^{2b}R^{2c}; and all other groups are as defined in the Summary of the Invention or in any of the embodiments. In certain embodiments, R² is ring (c); R^{2a} is alkyl; and all other groups are as defined in the Summary of the Invention or in any of the embodiments. In certain embodiments, R² is ring (c); R^{2a} is alkyl, cycloalkyl, halo, or —NR^{2b}R^{2c} where R^{2b} is hydrogen and R^{2c} is alkyl; and all other groups are as defined in the Summary of the Invention or in any of the embodiments. In certain embodiments, ring (c) is indolyl substituted on the nitrogen with R^{2a} and optionally substituted on the X¹ or X² carbon with R^{2a}; and all other groups are as defined in the Summary of the Invention or in any of the embodiments. In certain embodiments, ring (c) is indazolyl substituted with R^{2a} on the 1-position nitrogen and optionally substituted on the X² carbon with R^{2a} ; and all other groups are as defined in the Summary of the Invention or in any of the embodiments. In certain embodiments, ring (c) is indazolyl substituted with R^{2a} on the 2-position nitrogen and optionally substituted on the X² carbon with R^{2a}; and all other groups are as defined in the Summary of the Invention or in any of the embodiments. In certain embodiments, ring (c) is benzimidazolyl substituted with R^{2a} on the 1-position nitrogen and optionally substituted on the X¹ carbon with R^{2a}; and all other groups are as defined in the Summary of the Invention or in any of the embodiments. In certain embodiments, ring (c) is benzotriazolyl substituted on the X¹ nitrogen with R^{2a}; and all other groups are as defined in the Summary of the Invention or in any of the embodiments. In certain embodiments, ring (c) is benzotriazolyl substituted on the X² nitrogen with R^{2a}; and all other groups are as defined in the Summary of the Invention or in any of the embodiments.

In certain embodiments of the compounds described herein, R² is indazolyl or pyrazolopyridinyl where a carbon atom in R² is the point of attachment to the pyrimidinyl in Formula I and where R² is optionally substituted with 1, 2, or 3 R^{2a} groups; and all other groups are as defined in the Summary of the Invention or in any of the embodiments. In certain embodiments, R² is indazolyl or pyrazolopyridinyl where a carbon atom in R² is the point of attachment to the pyrimidinyl in Formula I and where R² is optionally substituted with 1 or 2 R^{2a} groups; and all other groups are as defined in the Summary of the Invention or in any of the embodiments. In certain embodiments, R² is indazol-5-yl, indazl-6-yl, pyrazolo[4,5-b]pyridin-6-yl, pyrazolo[5,4-d]pyridin-6-yl, pyrazolo[4,5-e]pyridin-6-yl, each of which is optionally substituted with 1 or 2 R^{2a} groups; and all other groups are as defined in the Summary of the Invention or in any of the embodiments. In certain embodiments, R² is indazol-5-yl or indazl-6-yl, each of which is optionally substituted with 1 or 2 R^{2a} groups; and all other groups are as defined in the Summary of the Invention or in any of the embodiments. In certain embodiments, R² is pyrazolo[4,5-b]pyridin-6-yl, pyrazolo[5,4-d]pyridin-6-yl, or pyrazolo[4,5-e]pyridin-6-yl, each of which is optionally substituted with 1 or 2 R^{2a} groups; and all other groups are as defined in the Summary of the Invention or in any of the embodiments.

In certain embodiments of the compounds described herein, R² is 2(3H)-oxo-benzoimidazolyl where a carbon atom in R² is the point of attachment to the pyrimidinyl in Formula I and where R² is optionally substituted with 1, 2, or 3 R^{2a} groups; and all other groups are as defined in the Summary of the Invention or in any of the embodiments.

In certain embodiments of the compounds described herein, R² is benzimidazolyl or imidazopyridinyl where a carbon atom in R² is the point of attachment to the pyrimidinyl in Formula I and where R² is optionally substituted with 1, 2, or 3 R^{2a} groups; and all other groups are as defined in the Summary of the Invention or in any of the embodiments. In certain embodiments, R² is benzimidazolyl or imidazopyridinyl where a carbon atom in R² is the point of attachment to the pyrimidinyl in Formula I and where R² is optionally substituted with 1 or 2 R^{2a} groups; and all other groups are as defined in the Summary of the Invention or in any of the embodiments. In certain embodiments, R² is benzimidazol-5-yl, benzimidazol-6-yl, imidazo[4,5-b]pyridin-5-yl, or imidazo[4,5-b]pyridin-6-yl, each of which is optionally substituted with 1 or 2 R^{2a} groups; and all other groups are as defined in the Summary of the Invention or in any of the embodiments. In certain embodiments, R² is benzimidazol-5-yl or benzimidazol-6-yl, each of which is optionally substituted with 1 or 2 R^{2a} groups; and all other groups are as defined in the Summary of the Invention or in any of the embodiments. In certain embodiments, R² is imidazo[4,5-b]pyridin-5-yl, or imidazo[4,5-b]pyridin-6-yl, each of which is optionally substituted with 1 or 2 R^{2a} groups; and all other groups are as defined in the Summary of the Invention or in any of the embodiments.

In certain embodiments of the compounds described herein, R² is indazolyl or benzimidazolyl where a carbon atom in R² is the point of attachment to the pyrimidinyl in Formula I and where R² is optionally substituted with 1, 2, or 3 R^{2a} groups; and all other groups are as defined in the Summary of the Invention or in any of the embodiments. In certain embodiments, R² is indazolyl or benzimidazolyl where a carbon atom in R² is the point of attachment to the pyrimidinyl in Formula I and where R² is optionally substituted with 1 or 2 R^{2a} groups; and all other groups are as defined in the Summary of the Invention or in any of the embodiments. In certain embodiments, R² is indazol-5-yl, indazl-6-yl, benzimidazol-5-yl, or benzimidazol-6-yl, each of which is optionally substituted with 1 or 2 R^{2a} groups; and all other groups are as defined in the Summary of the Invention or in any of the embodiments. In certain embodiments, R² is indazol-5-yl or indazl-6-yl, each of which is optionally substituted with 1 or 2 R^{2a} groups; and all other groups are as defined in the Summary of the Invention or in any of the embodiments. In certain embodiments, R² is benzimidazol-5-yl or benzimidazol-6-yl, each of which is optionally substituted with 1 or 2 R^{2a} groups; and all other groups are as defined in the Summary of the Invention or in any of the embodiments.

In certain embodiments of the compounds described herein, R² is substituted with one R^{2a}; and all other groups are as defined in the Summary of the Invention or in any of the embodiments. In certain embodiments, R² is substituted with one R^{2a} where R^{2a} is alkyl; hydroxyalkyl; alkoxyalkyl; halo; cycloalkyl; cycloalkylalkyl; heterocycloalkyl; heterocycloalkylalkyl optionally substituted with 1 alkyl; aminoalkyl; alkylaminoalkyl; dialkylaminoalkyl; 1,3-dioxo-isoindolinylalkyl; or —NR^{2b}R^{2c}; and all other groups are as defined in the Summary of the Invention or in any of the embodiments. In certain embodiments, R² is substituted with one R^{2a}; and all other groups are as defined in the Summary of the Invention or in any of the embodiments. In certain embodiments, R² is substituted with one R^{2a} where R^{2a} is alkyl; hydroxyalkyl; alkoxyalkyl; halo; cycloalkyl; cycloalkylalkyl; heterocycloalkyl; heterocycloalkylalkyl optionally substituted with 1 alkyl; aminoalkyl; alkylaminoalkyl; dialkylaminoalkyl; 1,3-dioxo-isoindolinylalkyl; or —NR^{2b}R^{2c} where R^{2b} is hydrogen or alkyl and R^{2c} is alkyl, alkoxylalkyl, dialkylaminoalkyl or heterocycloalkylalkyl; and all other groups are as defined in the Summary of the Invention or in any of the embodiments.

In certain embodiments of the compounds described herein, R² is substituted with one R^{2a} where R^{2a} is methyl; ethyl; hydroxyalkyl; methoxyalkyl; chloro; cyclopropyl; cyclobutyl; cyclopropylmethyl; oxetanyl; pyrrolidinyl; morpholinyl; piperazinyl; N-alkyl-piperazinyl; pyrrolidinylalkyl; morpholinylalkyl; N-alkyl-piperazinylalkyl; aminoalkyl; methylaminoalkyl; dimethylaminoalkyl; 1,3-dioxo-isoindolinylalkyl; or —NR^{2b}R^{2c} where R^{2b} is hydrogen or methyl and R^{2c} is methyl, methoxyalkyl, dimethylaminoalkyl, pyrrolidinylalkyl, or morpholinylalkyl; and all other groups are as defined in the Summary of the Invention or in any of the embodiments.

In certain embodiments of the compounds described herein, R² is substituted with two R^{2a}; and all other groups are as defined in the Summary of the Invention or in any of the embodiments. In certain embodiments, R² is substituted with two R^{2a} where one R^{2a} is alkyl, cycloalkylalkyl, or halo and a second R^{2a} is alkyl; hydroxyalkyl; cycloalkyl; cycloalkylalkyl; heterocycloalkyl; heterocycloalkylalkyl optionally substituted with 1 alkyl; aminoalkyl; alkylaminoalkyl; dialkylaminoalkyl; 1,3-dioxo-isoindolinylalkyl; or —NR^{2b}R^{2c}; and all other groups are as defined in the Summary of the Invention or in any of the embodiments. In certain embodiments, R² is substituted with two R^{2a} where one R^{2a} is alkyl, cycloalkylalkyl, or halo and a second R^{2a} is alkyl; hydroxyalkyl; cycloalkyl; cycloalkylalkyl; heterocycloalkyl; heterocycloalkylalkyl optionally substituted with 1 alkyl; aminoalkyl; alkylaminoalkyl; dialkylaminoalkyl; 1,3-dioxo-isoindolinylalkyl; or —NR^{2b}R^{2c} where R^{2b} is hydrogen or alkyl and R^{2c} is alkyl, alkoxylalkyl, dialkylaminoalkyl or heterocycloalkylalkyl; and all other groups are as defined in the Summary of the Invention or in any of the embodiments. In certain embodiments, R² is substituted with two R^{2a} where one R^{2a} is alkyl and a second R^{2a} is alkyl; hydroxyalkyl; cycloalkyl; cycloalkylalkyl; heterocycloalkyl; heterocycloalkylalkyl optionally substituted with 1 alkyl; aminoalkyl; alkylaminoalkyl; dialkylaminoalkyl; 1,3-dioxo-isoindolinylalkyl; or —NR^{2b}R^{2c} where R^{2b} is hydrogen or alkyl and R^{2c} is alkyl, alkoxylalkyl, dialkylaminoalkyl or heterocycloalkylalkyl; and all other groups are as defined in the Summary of the Invention or in any of the embodiments.

In certain embodiments of the compounds described herein, R² is substituted with two R^{2a} where one R^{2a} is methyl, cyclopropylmethyl, or chloro and a second R^{2a} is methyl; ethyl; 4-hydroxybutyl; cyclopropyl; cyclobutyl; cyclopropylmethyl; oxetanyl; pyrrolidinyl; morpholinyl; piperazinyl; N-alkyl-piperazinyl; pyrrolidinylalkyl; morpholinylalkyl; N-alkyl-piperazinylalkyl; methylaminoalkyl; methylaminoalkyl; dimethylaminoalkyl; 1,3-dioxo-isoindolinylalkyl; or —NR^{2b}R^{2c} where R^{2b} is hydrogen or methyl and R^{2c} is methyl, methoxyalkyl, dimethylaminoalkyl, pyrrolidinylalkyl, or morpholinylalkyl; and all other groups are as defined in the Summary of the Invention or in any of the embodiments. In certain embodiments, R² is substituted with two R^{2a} where one R^{2a} is methyl and a second R^{2a} is methyl; ethyl; 4-hydroxybutyl; cyclopropyl; cyclobutyl; cyclopropylmethyl; pyrrolidinyl; morpholinyl; piperazinyl; N-alkyl-piperazinyl; pyrrolidinylalkyl; morpholinylalkyl; N-alkyl-piperazinylalkyl; methylaminoalkyl; methylaminoalkyl; dimethylaminoalkyl; 1,3-dioxo-isoindolinylalkyl; or —NR^{2b}R^{2c} where R^{2b} is hydrogen or methyl and R^{2c} is methyl, methoxyalkyl, dimethylaminoalkyl, pyrrolidinylalkyl, or morpholinylalkyl; and all other groups are as defined in the Summary of the Invention or in any of the embodiments.

In certain embodiments of the compounds described herein, R² is substituted with one or two R^{2a} where R^{2a} is alkyl, aminoalkyl, alkylaminoalkyl, dialkylaminoalkyl, cycloalkyl, cycloalkylalkyl, or heterocycloalkylalkyl; and all other groups are as defined in the Summary of the Invention or in any of the embodiments. In certain embodiments, R^{2A} is alkyl; and all other groups are as defined in the Summary of the Invention or in any of the embodiments. In certain embodiments, R^{2A} is aminoalkyl, alkylaminoalkyl, dialkylaminoalkyl, or heterocycloalkylalkyl; and all other groups are as defined in the Summary of the Invention or in any of the embodiments. In certain embodiments, R^{2A} is aminoalkyl, alkylaminoalkyl, or dialkylaminoalkyl; and all other groups are as defined in the Summary of the Invention or in any of the embodiments. In certain embodiments, R^{2A} is heterocycloalkylalkyl; and all other groups are as defined in the Summary of the Invention or in any of the embodiments. In certain embodiments, R^{2A} is cycloalkyl or cycloalkylalkyl; and all other groups are as defined in the Summary of the Invention or in any of the embodiments.

In certain embodiments of the compounds described herein, at least one of the 1, 2, or 3 R^{2a} is alkyl; all other groups are as defined in the Summary of the Invention or in any of the embodiments. In certain embodiments, at least one of the 1, 2, or 3 R^{2a} is heterocycloalkyl; all other groups are as defined in the Summary of the Invention or in any of the embodiments. In certain embodiments, at least one of the 1, 2, or 3 R^{2a} is heterocycloalkylalkyl; all other groups are as defined in the Summary of the Invention or in any of the embodiments. In certain embodiments, at least one of the 1, 2, or 3 R^{2a} is aminoalkyl, alkylaminoalkyl, or dialkylaminoalkyl; all other groups are as defined in the Summary of the Invention or in any of the embodiments. In certain embodiments, at least one of the 1, 2, or 3 R^{2a} is —NR^{2b}R^{2c}; all other groups are as defined in the Summary of the Invention or in any of the embodiments.

In certain embodiments of the compounds described herein, R² is

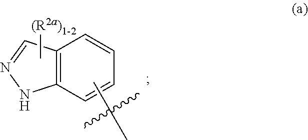

where a carbon atom in R² is the point of attachment to the pyrimidinyl in Formula I and all other groups are as defined in the Summary of the Invention or in any of the embodiments. In certain embodiments, R² is (a) and is attached by the carbon in the 5- or 6-position of the (a) ring; and all other groups are as defined in the Summary of the Invention or in any of the embodiments.

In certain embodiments of the compounds described herein, R² is

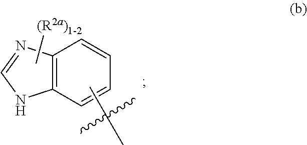

and all other groups are as defined in the Summary of the Invention or in any of the embodiments. In certain embodiments, R² is (b) and is attached by the carbon in the 5- or 6-position of the (b) ring; and all other groups are as defined in the Summary of the Invention or in any of the embodiments.

In certain embodiments, the compound of Formula I is according to Formula I(a):

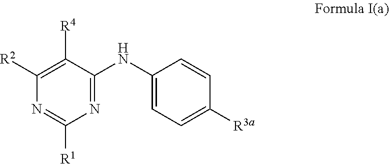

where all other groups are as defined in the Summary of the Invention or in any of the embodiments.

In certain embodiments of the compounds described herein, R¹ is morpholin-4-yl; and all other groups are as defined in the Summary of the Invention or in any of the embodiments.

In certain embodiments of the compounds described herein, R^{3a} is cyano, halo, alkyl, alkoxycarbonyl, heterocycloalkyl, alkylcarbonyl, alkylsulfinyl, alkylsulfonyl, alkoxy, —NR^{3b}R^{3a}, —C(O)NR^{3b}R^{3a}, —S(O)₂NR^{3b}R^{3c}, or 5-membered heteroaryl optionally substituted with 1 or 2 R⁵ groups; and all other groups are as defined in the Summary of the Invention or in any of the embodiments.

In certain embodiments of the compounds described herein, R^{3a} is halo; alkyl; methoxy; methylcarbonyl; alkylsulfinyl; alkylsulfonyl; cyano; alkoxycarbonyl; —NR^{3b}R^{3c} where R^{3b} is hydrogen and R^{3c} is alkylcarbonyl, alkylsulfonyl, or alkoxycarbonyl; —C(O)NR^{3b}R^{3c} where R^{3b} is hydrogen or alkyl and R^{3c} is hydrogen, alkyl, cycloalkyl (optionally substituted with 1 alkyl), or cycloalkylmethyl; —S(O)₂NR^{3b}R^{3c} where R^{3b} is hydrogen and R^{3c} is hydrogen or alkyl; 5-membered heterocycloalkyl; or a 5-membered heteroaryl optionally substituted with 1 R⁵ groups; and all other groups are as defined in the Summary of the Invention or in any of the embodiments.

In certain embodiments of the compounds described herein, R^{3a} is halo; and all other groups are as defined in the Summary of the Invention or in any of the embodiments. In certain embodiments, R^{3a} is chloro; and all other groups are as defined in the Summary of the Invention or in any of the embodiments.

In certain embodiments of the compounds described herein, R^{3a} is —C(O)NR^{3b}R^{3c}; and all other groups are as defined in the Summary of the Invention or in any of the embodiments. In certain embodiments, Rⁱa is —C(O)NR^{3b}R^{3c} and R^{3b} and R^{3c} are independently hydrogen or alkyl; and all other groups are as defined in the Summary of the Invention or in any of the embodiments.

In certain embodiments of the compounds described herein, R^{3a} is a 5-membered heteroaryl optionally substituted with 1 R⁵; and all other groups are as defined in the Summary of the Invention or in any of the embodiments. In certain embodiments, R^{3a} is triazolyl, oxazolyl, imidazolyl, oxadiazolyl, pyrazolyl, or pyrrolyl, each of which is optionally substituted with 1 R⁵; and all other groups are as defined in the Summary of the Invention or in any of the embodiments. In certain embodiments, R^{3a} is triazolyl, oxazolyl, imidazolyl, oxadiazolyl, pyrazolyl, or pyrrolyl, each of which is substituted with one alkyl, halo, haloalkyl, cycloalkyl, or phenylmethyl (substituted with one alkoxy); and all other groups are as defined in the Summary of the Invention or in any of the embodiments. In certain embodiments, R^{3a} is triazolyl, oxazolyl, imidazolyl, oxadiazolyl, pyrazolyl, or pyrrolyl, each of which is substituted with one methyl, isopropyl, fluoro, trifluoromethyl, cyclopropyl, or phenylmethyl (substituted with methoxy); and all other groups are as defined in the Summary of the Invention or in any of the embodiments.

In certain embodiments of the compounds described herein, R^{3a} is a 5-membered heteroaryl with 1, 2, or 3 ring nitrogen atoms, optionally substituted with one or two R⁵ groups; and all other groups are as defined in the Summary of the Invention or in any of the embodiments. In certain embodiments, R^{3a} is a 5-membered heteroaryl with 1 or 2 ring nitrogen atoms, optionally substituted with one or two R⁵ groups; and all other groups are as defined in the Summary of the Invention or in any of the embodiments. In certain embodiments, R^{3a} is triazolyl, oxazolyl, imidazolyl, oxadiazolyl, pyrazolyl, or pyrrolyl, each of which is optionally substituted with one or two R⁵ groups; and all other groups are as defined in the Summary of the Invention or in any of the embodiments. In certain embodiments, R^{3a} is triazolyl optionally substituted with one or two R⁵ groups; and all other groups are as defined in the Summary of the Invention or in any of the embodiments. In certain embodiments, R^{3a} is oxazolyl or oxadiazolyl, each of which is optionally substituted with one or two R⁵ groups; and all other groups are as defined in the Summary of the Invention or in any of the embodiments. In certain embodiments, R^{3a} is triazolyl, imidazolyl, pyrazolyl, or pyrrolyl, each of which is optionally substituted with one or two R⁵ groups; and all other groups are as defined in the Summary of the Invention or in any of the embodiments. In certain embodiments, R^{3a} is imidazolyl, pyrazolyl, or pyrrolyl, each of which is optionally substituted with one or two R⁵ groups; and all other groups are as defined in the Summary of the Invention or in any of the embodiments.

In certain embodiments of the compounds described herein, R⁴ is hydrogen; and all other groups are as defined in the Summary of the Invention or in any of the embodiments. In certain embodiments, R⁴ is methyl; and all other groups are as defined in the Summary of the Invention or in any of the embodiments. In certain embodiments, R⁴ is fluoro; and all other groups are as defined in the Summary of the Invention or in any of the embodiments. In certain embodiments, R⁴ is —CN; and all other groups are as defined in the Summary of the Invention or in any of the embodiments.

In certain embodiments of the compounds described herein, R³ is heteroaryl optionally substituted with 1, 2, or 3 R^{3a} groups; and all other groups are as defined in the Summary of the Invention or in any of the embodiments. In certain embodiments, R³ is a 6-10 membered heteroaryl optionally substituted with 1, 2, or 3 R^{3a} groups; and all other groups are as defined in the Summary of the Invention or in any of the embodiments. In certain embodiments, R³ is a 5-9 membered heteroaryl optionally substituted with 1, 2, or 3 R^{3a} groups; and all other groups are as defined in the Summary of the Invention or in any of the embodiments. In certain embodiments, R³ is a 5-6 membered or a 9 membered heteroaryl optionally substituted with 1, 2, or 3 R^{3a} groups; and all other groups are as defined in the Summary of the Invention or in any of the embodiments. In certain embodiments, R³ is a 6-membered heteroaryl optionally substituted with 1, 2, or 3 R^{3a} groups; and all other groups are as defined in the Summary of the Invention or in any of the embodiments. In certain embodiments, R³ is a 9-membered heteroaryl optionally substituted with 1, 2, or 3 R^{3a} groups; and all other groups are as defined in the Summary of the Invention or in any of the embodiments. In certain embodiments, R³ is a 9-membered heteroaryl with 1, 2, or 3 nitrogen atoms where the heteroaryl is optionally substituted with 1, 2, or 3 R^{3a} groups; and all other groups are as defined in the Summary of the Invention or in any of the embodiments. In certain embodiments, R³ is pyridinyl, indolyl, benzoisoxazolyl, indazolyl, benzotriazolyl, benzoxazolyl, or benzimidazolyl, each of which is optionally substituted with 1, 2, or 3 R^{3a} groups; and all other groups are as defined in the Summary of the Invention or in any of the embodiments. In certain embodiments, R³ is pyridin-3-yl, pyridin-4-yl, indol-4-yl, indol-5-yl, indol-6-yl, benzoisoxazolyl, indazol-5-yl, indazol-6-yl, benzotriazol-5-yl, benzotriazol-6-yl, benzoxazol-6-yl, benzimidazol-5-yl, or benzimidazol-6-yl, each of which is optionally substituted with 1, 2, or 3 R^{3c} groups; and all other groups are as defined in the Summary of the Invention or in any of the embodiments. In certain embodiments, R³ is pyridinyl, indolyl, indazolyl, benzotriazolyl, or benzimidazolyl, each of which is optionally substituted with 1, 2, or 3 Rⁱa groups; and all other groups are as defined in the Summary of the Invention or in any of the embodiments. In certain embodiments, R³ is pyridin-3-yl, indol-5-yl, indol-6-yl, indazol-5-yl, indazol-6-yl, benzotriazol-5-yl, benzotriazol-6-yl, benzimidazol-5-yl, or benzimidazol-6-yl, each of which is optionally substituted with 1, 2, or 3 R^{3c} groups; and all other groups are as defined in the Summary of the Invention or in any of the embodiments.

In certain embodiments of the compounds described herein, R³ is a 6-10 membered heteroaryl; or R³ is pyridinyl; or R³ is a 9-membered heteroaryl; where R³ is optionally substituted with 1, 2, or 3 R^{3a} groups and each R^{3a} is independently alkyl, cycloalkyl, alkoxy, halo, —NR^{3b}R^{3a}, or —C(O)NR^{3b}R^{3c}; and all other groups are as defined in the Summary of the Invention or in any of the embodiments.

In certain embodiments of the compounds described herein, R³ is a 6-10 membered heteroaryl; or R³ is pyridinyl; or R³ is a 9-membered heteroaryl; where R³ is optionally substituted with 1 R^{3a} groups and each R^{3c} is alkyl; cycloalkyl; alkoxy; halo; —NR^{3b}R^{3c} where R^{3b} is hydrogen and R^{3c} is alkyl; or —C(O)NR^{3b}R^{3c} where R^{3b} is hydrogen and R^{3c} is alkyl or cycloalkyl; and all other groups are as defined in the Summary of the Invention or in any of the embodiments.

In certain embodiments of the compounds described herein, R³ is a 6-10 membered heteroaryl; or R³ is pyridinyl; or R³ is a 9-membered heteroaryl; where R³ is optionally substituted with 2 R^{3a} groups independently selected from alkyl and halo; and all other groups are as defined in the Summary of the Invention or in any of the embodiments.

In certain embodiments of the compounds described herein, R³ is a 6-10 membered heteroaryl; or R³ is pyridinyl; or R³ is a 9-membered heteroaryl; where R³ is optionally substituted with 3 R^{3a} groups where R^{3a} is alkyl; and all other groups are as defined in the Summary of the Invention or in any of the embodiments.

In certain embodiments of the compounds described herein, R³ is a 6-10 membered heteroaryl; or R³ is pyridinyl; or R³ is a 9-membered heteroaryl; where R³ is optionally substituted with 1 R^{3a} group which is alkyl; and all other groups are as defined in the Summary of the Invention or in any of the embodiments.

In certain embodiments of the compounds described herein, R³ is a 6-10 membered heteroaryl; or R³ is pyridinyl; or R³ is a 9-membered heteroaryl; where R³ is optionally substituted with 1 R^{3a} group which is —NR^{3b}R^{3c} or —C(O)NR^{3b}R^{3c}; and all other groups are as defined in the Summary of the Invention or in any of the embodiments.

In certain embodiments of the compounds described herein, R³ is a 6-10 membered heteroaryl; or R³ is pyridinyl; or R³ is a 9-membered heteroaryl; where R³ is optionally substituted with 1 R^{3a} group which is alkoxy; and all other groups are as defined in the Summary of the Invention or in any of the embodiments.

In certain embodiments of the compounds described herein, R³ is

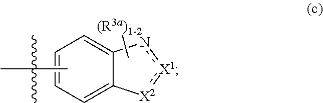

where one of the bonds in

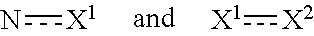

is a single bond and the other is a double bond, X¹ and X² are independently N, NR^{3a}, CH, or CR^{3a}; and all other groups are as defined in the Summary of the Invention or in any of the embodiments. In certain embodiments, R³ is ring (c) and is attached by a carbon in the 5- or 6-position of ring (c) to the —NH— group; and all other groups are as defined in the Summary of the Invention or in any of the embodiments. In certain embodiments, R³ is ring (c); R^{3a} is alkyl, cycloalkyl, halo, or —NR^{3b}R^{3c}; and all other groups are as defined in the Summary of the Invention or in any of the embodiments. In certain embodiments, R³ is ring (c); R^{3a} is alkyl; and all other groups are as defined in the Summary of the Invention or in any of the embodiments. In certain embodiments, R³ is ring (c); R^{3c} is alkyl, cycloalkyl, halo, or —NR^{3b}R^{3c} where R^{ab} is hydrogen and R^{3c} is alkyl; and all other groups are as defined in the Summary of the Invention or in any of the embodiments. In certain embodiments, ring (c) is indolyl substituted on the nitrogen with R^{3a} and optionally substituted on the X¹ or X² carbon with R^{3a}; and all other groups are as defined in the Summary of the Invention or in any of the embodiments. In certain embodiments, ring (c) is indazolyl substituted with R^{3a} on the 1-position nitrogen and optionally substituted on the X² carbon with R^{3a}; and all other groups are as defined in the Summary of the Invention or in any of the embodiments. In certain embodiments, ring (c) is indazolyl substituted with R^{3a} on the 2-position nitrogen and optionally substituted on the X² carbon with R^{3a}; and all other groups are as defined in the Summary of the Invention or in any of the embodiments. In certain embodiments, ring (c) is benzimidazolyl substituted with R^{3a} on the 1-position nitrogen and optionally substituted on the X¹ carbon with R^{3a}; and all other groups are as defined in the Summary of the Invention or in any of the embodiments. In certain embodiments, ring (c) is benzotriazolyl substituted on the X¹ nitrogen with R^{3a}; and all other groups are as defined in the Summary of the Invention or in any of the embodiments. In certain embodiments, ring (c) is benzotriazolyl substituted on the X² nitrogen with R^{3a}; and all other groups are as defined in the Summary of the Invention or in any of the embodiments.

In certain embodiments, the Compound of Formula I is according to Formula I(b):

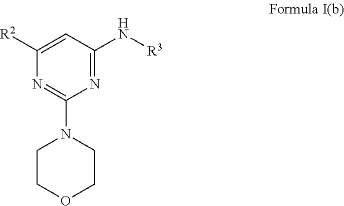

where all other groups are as defined in the Summary of the Invention or in any of the embodiments.

In certain embodiments, the Compound of Formula I is according to Formula I(c):

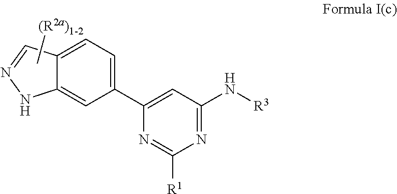

where all other groups are as defined in the Summary of the Invention or in any of the embodiments.

In certain embodiments, the compound of Formula I is according to Formula I(d):

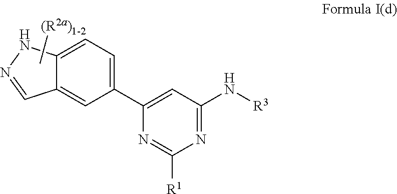

where all other groups are as defined in the Summary of the Invention or in any of the embodiments.

In certain embodiments, the compound of Formula I is according to Formula I(e):

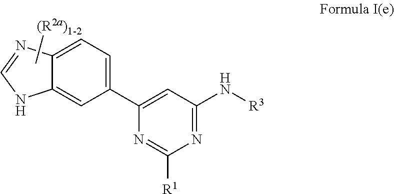

where all other groups are as defined in the Summary of the Invention or in any of the embodiments.

In certain embodiments, the compound of Formula I is according to Formula I(f):

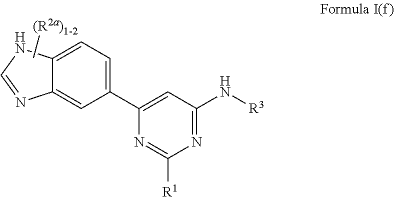

where all other groups are as defined in the Summary of the Invention or in any of the embodiments.

In certain embodiments, the compound of Formula I is according to Formula I(g):

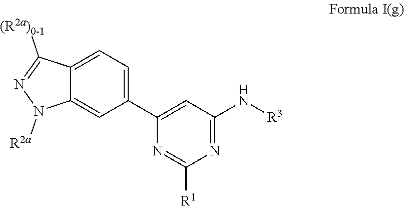

where all other groups are as defined in the Summary of the Invention or in any of the embodiments.

In certain embodiments, the compound of Formula I is according to Formula I(h):

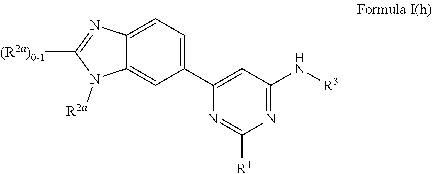

where all other groups are as defined in the Summary of the Invention or in any of the embodiments.

In certain embodiments of the compounds described herein, R¹ is morpholin-4-yl; and all other groups are as defined in the Summary of the Invention or in any of the embodiments.

In certain embodiments of the compounds described herein, R³ is phenyl optionally substituted with 1, 2, or 3 R^{3a} groups, or with 1 or 2 R^{3a} groups, or with 1 R^{3a} group; and all other groups are as defined in the Summary of the Invention or in any of the embodiments.

In certain embodiments of the compounds described herein, R³ is phenyl substituted with 1 R^{3a} group or R³ is phenyl substituted with 1 R^{3a} group at the para position; and R^{3a} is cyano, halo, alkyl, alkoxycarbonyl, heterocycloalkyl, alkylcarbonyl, alkylsulfinyl, alkylsulfonyl, alkoxy, —NR^{3b}R^{3a}, —C(O)NR^{3b}R^{3a}, —S(O)₂NR^{3b}R^{3a}, or 5-membered heteroaryl optionally substituted with 1 or 2 R⁵ groups; or R^{3a} is halo, alkyl, methoxy, methylcarbonyl, alkylsulfinyl, alkylsulfonyl, cyano, alkoxycarbonyl, —NHR^{3c}, —C(O)NR^{3b}R^{3c}; —S(O)₂NR^{3b}R^{3c}, 5-membered heterocycloalkyl, or a 5-membered heteroaryl optionally substituted with 1 R⁵ groups; where for —NHR^{3a}, R^{3c} is alkylcarbonyl, alkylsulfonyl, or alkoxycarbonyl; and for —C(O)NR^{3b}R^{3c}, R^{3b} is hydrogen or alkyl and R^{3c} is hydrogen, alkyl, cycloalkylmethyl, or cycloalkyl (where cycloalkyl is optionally substituted with 1 alkyl); and for —S(O)₂NR^{3b}R^{3a}, R^{3b} is hydrogen and R^{3c} is hydrogen or alkyl; or R^{3a} is halo, alkoxycarbonyl, —NR^{3b}R^{3a}, —C(O)NR^{3b}R^{3c}, or 5-membered heteroaryl optionally substituted with 1 or 2 R⁵ groups; or R^{3c} is halo, alkoxycarbonyl, —C(O)NR^{3b}R^{3a}, or 5-membered heteroaryl optionally substituted with 1 or 2 R⁵ groups; or R^{3a} is halo; or R^{3a} is chloro; or R^{3a} is —C(O)NR^{3b}R^{3c}; or R^{3c} is —C(O)NR^{3b}R^{3c}, where R^{3b} and R^{3c} are independently hydrogen or alkyl; or R^{3a} is a 5-membered heteroaryl optionally substituted with 1 R⁵; or R^{3a} is triazolyl, oxazolyl, imidazolyl, oxadiazolyl, pyrazolyl, or pyrrolyl, each of which is optionally substituted with 1 R⁵; or R^{3a} is triazolyl, oxazolyl, imidazolyl, oxadiazolyl, pyrazolyl, or pyrrolyl, each of which is substituted with one alkyl, halo, haloalkyl, cycloalkyl, or phenylmethyl (optionally substituted with one alkoxy); or R^{3a} is triazolyl, oxazolyl, imidazolyl, oxadiazolyl, pyrazolyl, or pyrrolyl, each of which is substituted with one methyl, isopropyl, fluoro, trifluoromethyl, cyclopropyl, or phenylmethyl (substituted with methoxy); and all other groups are as defined in the Summary of the Invention or in any of the embodiments.

In certain embodiments of the compounds described herein, R³ is pyridinyl, indolyl, benzoisoxazolyl, indazolyl, benzotriazolyl, benzoxazolyl, or benzimidazolyl, each of which is optionally substituted with 1, 2, or 3 R^{3c} groups; or R³ is pyridin-3-yl, pyridin-4-yl, indol-4-yl, indol-5-yl, indol-6-yl, benzoisoxazolyl, indazol-5-yl, indazol-6-yl, benzotriazol-5-yl, benzotriazol-6-yl, benzoxazol-6-yl, benzimidazol-5-yl, or benzimidazol-6-yl, each of which is optionally substituted with 1, 2, or 3 R^{3a} groups; or R³ is pyridinyl, indolyl, indazolyl, benzotriazolyl, or benzimidazolyl, each of which is optionally substituted with 1, 2, or 3 R^{3a} groups; or R³ is pyridin-3-yl, indol-5-yl, indol-6-yl, indazol-5-yl, indazol-6-yl, benzotriazol-5-yl, benzotriazol-6-yl, benzimidazol-5-yl, or benzimidazol-6-yl, each of which is optionally substituted with 1, 2, or 3 R^{3a} groups; and all other groups are as defined in the Summary of the Invention or in any of the embodiments.

In certain embodiments of the compounds described herein, R³ is phenyl substituted at the para position with one halo, cyano, alkylcarbonyl, alkylsulfonyl, alkylsulfinyl, alkoxy, alkoxycarbonyl, alkyl, —NR^{3b}R^{3c}, —C(O)NR^{3b}R^{3c}, —S(O)₂NR^{3b}R^{3c}, or 5-membered heteroaryl optionally substituted with 1 or 2 R⁵ groups; or R³ is pyridinyl substituted with one alkoxy or —C(O)NR^{3b}R^{3c}; or R³ is a 9-membered heteroaryl substituted with one alkyl or cycloalkyl; or R³ is a 9-membered heteroaryl substituted with 2 or 3 alkyl; and all other groups are as defined in the Summary of the Invention or in any of the embodiments. In certain embodiments, R³ is phenyl substituted at the para position with one halo, cyano, carboxy, alkylcarbonyl, alkylsulfonyl, alkylsulfinyl, alkoxy, alkoxycarbonyl, alkyl, —NR^{3b}R^{3c}, —C(O)NR^{3b}R^{3c}, —S(O)₂NR^{3b}R^{3c}, or 5-membered heteroaryl optionally substituted with 1 or 2 R⁵ groups; or R³ is pyridinyl substituted with one alkoxy or —C(O)NR^{3b}R^{3c}; or R³ is a 9-membered heteroaryl substituted with one alkyl or cycloalkyl; or R³ is a 9-membered heteroaryl substituted with 2 or 3 alkyl; and R^{3b} is hydrogen or alkyl and R^{3c} is hydrogen, alkyl, cycloalkyl, cycloalkylalkyl, alkylsulfonyl, alkylcarbonyl, or alkoxycarbonyl; and all other groups are as defined in the Summary of the Invention or in any of the embodiments. In certain embodiments, R³ is phenyl substituted at the para position with one halo, carboxy, alkoxycarbonyl, —NR^{3b}R^{3c}, —C(O)NR^{3b}R^{3c}, or 5-membered heteroaryl optionally substituted with 1 or 2 R⁵ groups; or R³ is pyridinyl substituted with one alkoxy or —C(O)NR^{3b}R^{3c}; or R³ is a 9-membered heteroaryl substituted with one alkyl or cycloalkyl; or R³ is a 9-membered heteroaryl substituted with 2 or 3 alkyl; and all other groups are as defined in the Summary of the Invention or in any of the embodiments. In certain embodiments, R³ is phenyl substituted at the para position with one halo, carboxy, alkoxycarbonyl, —C(O)NR^{3b}R^{3a}, or 5-membered heteroaryl optionally substituted with 1 or 2 R⁵ groups; or R³ is pyridinyl substituted with one —C(O)NR^{3b}R^{3c}; or R³ is a 9-membered heteroaryl substituted with one alkyl or cycloalkyl; or R³ is a 9-membered heteroaryl substituted with 2 or 3 alkyl; and all other groups are as defined in the Summary of the Invention or in any of the embodiments.

In certain embodiments of the compounds described herein, R³ is pyridinyl substituted with one alkoxy or —C(O)NR^{3b}R^{3c}; or R³ is a 9-membered heteroaryl substituted with one alkyl or cycloalkyl; or R³ is a 9-membered heteroaryl substituted with 2 or 3 alkyl; and R^{3b} optionally is hydrogen or alkyl and R^{3c} is optionally hydrogen, alkyl, cycloalkyl, cycloalkylalkyl, alkylsulfonyl, alkylcarbonyl, or alkoxycarbonyl; and all other groups are as defined in the Summary of the Invention or in any of the embodiments.

In certain embodiments of the compounds described herein, R² is ring (a) or ring (b); and all other groups are as defined in the Summary of the Invention or in any of the embodiments.

In certain embodiments of the compounds described herein, R² is substituted with 1 or 2 R^{2a} where each R^{2a} is independently alkyl, cycloalkyl, cycloalkylalkyl, aminoalkyl, alkylaminoalkyl, dialkylaminoalkyl, alkoxyalkyl, heterocycloalkyl, or heterocycloalkylalkyl; and all other groups are as defined in the Summary of the Invention or in any of the embodiments.

In certain embodiments of the compounds described herein, R² is substituted with one R^{2a} where R^{2a} is alkyl, aminoalkyl, alkylaminoalkyl, dialkylaminoalkyl, or heterocycloalkylalkyl; and all other groups are as defined in the Summary of the Invention or in any of the embodiments.

In certain embodiments of the compounds described herein, R² is substituted with two R^{2a} groups, where one R^{2a} is alkyl, heterocycloalkyl, cycloalkyl, or cycloalkylalkyl and the other R^{2a} is alkyl, aminoalkyl, alkylaminoalkyl, dialkylaminoalkyl, alkoxyalkyl, heterocycloalkyl, or heterocycloalkylalkyl; and all other groups are as defined in the Summary of the Invention or in any of the embodiments.

In certain embodiments of the compounds described herein, R² is substituted with 1 or 2 R^{2a} groups, where one R^{2a} is alkyl and the other R^{2a}, when present, is aminoalkyl, alkylaminoalkyl, dialkylaminoalkyl, alkoxyalkyl, cycloalkyl, cycloalkylalkyl, heterocycloalkyl, or heterocycloalkylalkyl; and all other groups are as defined in the Summary of the Invention or in any of the embodiments.

In certain embodiments of the compounds described herein, R² is substituted with two R^{2a} groups, where one R^{2a} is alkyl and the other R^{2a} is aminoalkyl, alkylaminoalkyl, dialkylaminoalkyl, alkoxyalkyl, cycloalkyl, cycloalkylalkyl, heterocycloalkyl, or heterocycloalkylalkyl; and all other groups are as defined in the Summary of the Invention or in any of the embodiments.

In certain embodiments, the compound of Formula I is according to Formula I(i):

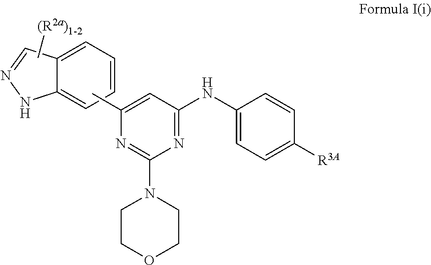

where all other groups are as defined in the Summary of the Invention or in any of the embodiments.

In certain embodiments, the compound of Formula I(i) is that where:
each R^{2a} is independently selected from halo, alkyl, alkenyl, alkylsulfonyloxyalkyl, alkoxyalkyl, aminoalkyl, alkylaminoalkyl, dialkylaminoalkyl, hydroxyalkyl, cycloalkyl, cycloalkylalkyl, heterocycloalkyl, heterocycloalkylalkyl, 1,3-dioxo-isoindolinylalkyl, and —NR^{2b}R^{2c}; where each heterocycloalkyl, either alone or as part of another group, is optionally substituted with 1 alkyl;
R^{2b} is hydrogen or alkyl;
R^{2c} is hydrogen, alkyl, aminoalkyl, alkylaminoalkyl, dialkylaminoalkyl, alkoxyalkyl, or heterocycloalkylalkyl; where the heterocycloalkyl in heterocycloalkylalkyl is optionally substituted with 1 or 2 groups independently selected from alkyl, hydroxy, alkylcarbonyl, and alkoxycarbonyl;
each R^{3a} is independently selected from —C(═NH)NHOH, cyano, halo, alkyl, alkylcarbonyl, alkoxycarbonyl, cycloalkyl, alkylsulfinyl, alkylsulfonyl, —OR^{3d}, —NR^{3b}R^{3c}, —C(O)NR^{3b}R^{3a}, —S(O)₂NR^{3b}R^{3a}, heterocycloalkyl, and heteroaryl optionally substituted with 1 R⁵ groups; where the heterocycloalkyl is optionally substituted with 1 alkyl;
R^{3b} is hydrogen or alkyl; R^{3c} is hydrogen, alkyl, alkylcarbonyl, alkylsulfonyl, alkoxycarbonyl, cycloalkylalkyl, or cycloalkyl optionally substituted with 1 alkyl;
R^{3d} is alkyl, methyl, or fluoro;
each R⁵ is independently halo, alkoxycarbonyl, alkyl, haloalkyl, cycloalkyl, or phenylmethyl which is optionally substituted with 1 alkoxy.

In certain embodiments, the compound of Formula I is according to Formula I(j):

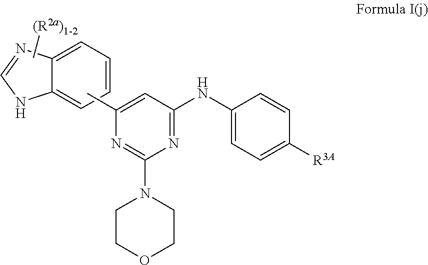

where all other groups are as defined in the Summary of the Invention or in any of the embodiments.

In certain embodiments, the compound of Formula I(j) is that where:
each R^{2a} is independently selected from halo, alkyl, alkenyl, alkylsulfonyloxyalkyl, alkoxyalkyl, aminoalkyl, alkylaminoalkyl, dialkylaminoalkyl, hydroxyalkyl, cycloalkyl, cycloalkylalkyl, heterocycloalkyl, heterocycloalkylalkyl, 1,3-dioxo-isoindolinylalkyl, and —NR^{2b}R^{2c}; where each heterocycloalkyl, either alone or as part of another group, is optionally substituted with 1 alkyl;
R^{2b} is hydrogen or alkyl;
R^{2c} is hydrogen, alkyl, aminoalkyl, alkylaminoalkyl, dialkylaminoalkyl, alkoxyalkyl, or heterocycloalkylalkyl; where the heterocycloalkyl in heterocycloalkylalkyl is optionally substituted with 1 or 2 groups independently selected from alkyl, hydroxy, alkylcarbonyl, and alkoxycarbonyl;
each R^{3c} is independently selected from —C(═NH)NHOH, cyano, halo, alkyl, alkylcarbonyl, alkoxycarbonyl, cycloalkyl, alkylsulfinyl, alkylsulfonyl, —OR^{3d}, —NR^{3b}R^{3a}, —C(O)NR^{3b}R^{3a}, —S(O)₂NR^{3b}R^{3c}, heterocycloalkyl, and heteroaryl optionally substituted with 1 R⁵ groups; where the heterocycloalkyl is optionally substituted with 1 alkyl;
R^{3b} is hydrogen or alkyl; R^{3c} is hydrogen, alkyl, alkylcarbonyl, alkylsulfonyl, alkoxycarbonyl, cycloalkylalkyl, or cycloalkyl optionally substituted with 1 alkyl;
R^{3d} is alkyl, methyl, or fluoro; and
each R⁵ is independently halo, alkoxycarbonyl, alkyl, haloalkyl, cycloalkyl, or phenylmethyl which is optionally substituted with 1 alkoxy.

In certain embodiments, the compound of Formula I is according to Formula I(k):

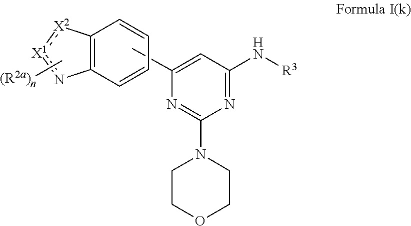

wherein:
n is 1 or 2;
where one of the bonds in

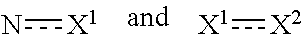

is a single pond and the other is a double bond, X¹ and X² are independently N, NR²a, CH, or CR^{2a};
each R^{2a} is independently selected from cyano, nitro, halo, hydroxy, alkyl, alkenyl, carboxy, alkoxycarbonyl, alkylcarbonyl, alkylsulfinyl, alkylsulfonyl, alkylsulfonyloxyalkyl, alkoxyalkyl, alkoxyalkenyl, aminoalkyl, alkylaminoalkyl, dialkylaminoalkyl, hydroxyalkyl, cycloalkyl, cycloalkylalkyl, heterocycloalkyl, heterocycloalkylalkyl, 1,3-dioxo-isoindolinylalkyl, —NR^{2b}R^{2c}, and —OR^{2d}; where each heterocycloalkyl, either alone or as part of another group, is optionally substituted with 1, 2, 3, or 4 groups independently selected from alkyl, hydroxy, alkylcarbonyl, and alkoxycarbonyl;
R^{2b} is hydrogen or alkyl;
R^{2c} is hydrogen, alkyl, aminoalkyl, alkylaminoalkyl, dialkylaminoalkyl, alkoxyalkyl, hydroxyalkyl, alkylcarbonyl, cycloalkylcarbonyl, alkylsulfonyl, alkoxycarbonyl, cycloalkylalkyl, or heterocycloalkylalkyl; where the heterocycloalkyl in heterocycloalkylalkyl is optionally substituted with 1 or 2 groups independently selected from alkyl, hydroxy, alkylcarbonyl, and alkoxycarbonyl;
R^{2d} is alkyl, haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl; where each heterocycloalkyl, either alone or as part of another group, is optionally substituted with 1 or 2 groups independently selected from alkyl, hydroxy, alkylcarbonyl, and alkoxycarbonyl;
R³ is phenyl or heteroaryl each of which is optionally substituted with 1, 2, or 3 R^{3a} groups;
each R^{3c} is independently selected from —C(═NH)NHOH, cyano, nitro, halo, hydroxy, alkyl, alkoxycarbonyl, alkoxyalkyl, cycloalkyl, cycloalkylalkyl, heterocycloalkyl, heterocycloalkylalkyl, alkylcarbonyl, alkylsulfinyl, alkylsulfonyl, —OR^{3d}, —NR^{3b}R^{3c}, —C(O)NR^{3b}R^{3a}, —S(O)₂NR^{3b}R^{3c}, and heteroaryl optionally substituted with 1, 2, or 3 R⁵ groups; where the heterocycloalkyl either alone or as part of heterocycloalkylalkyl is optionally substituted with 1 or two alkyl groups;
R^{3b} is hydrogen or alkyl;
R^{3c} is hydrogen, alkyl, haloalkyl, aminoalkyl, alkylaminoalkyl, dialkylaminoalkyl, alkoxyalkyl, alkylcarbonyl, alkylsulfonyl, alkoxycarbonyl, cycloalkylalkyl, heterocycloalkyl, heterocycloalkylalkyl, or cycloalkyl optionally substituted with 1 or 2 alkyl; or R^{3b} and R^{3c} together with the nitrogen to which they are attached form heterocycloalkyl;
R^{3d} is alkyl, haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl;
R⁴ is hydrogen, methyl, halo, or —CN; and
each R⁵ is independently halo, hydroxy, alkoxycarbonyl, alkyl, haloalkyl, cycloalkyl, cycloalkylalkyl, heterocycloalkyl, heterocycloalkylalkyl, phenyl, or phenylmethyl which is optionally substituted with 1 or 2 alkoxy;
optionally as a single stereoisomer or mixture of stereoisomers thereof and additionally optionally as a pharmaceutically acceptable salt thereof;
   provided that:
a) when R³ is pyrazolyl substituted with one R^{3a}, then R^{3a} is not cyclopropyl; and
b) when R³ is phenyl substituted with one R^{3a}, then the one R^{3a} is not 3-7-membered cycloalkyl ring.

In certain embodiments, the compound is according to Formula II

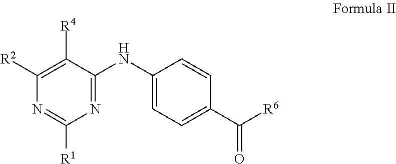

where
R¹ is heterocycloalkyl optionally substituted with 1, 2, 3, or 4 alkyl;
R² is a 9-membered bicyclic ring comprising 1, 2, 3, or 4 nitrogen atoms where one or both of the rings is aromatic, where a carbon atom in R² is the point of attachment to the pyrimidinyl in Formula I, and where R² is optionally substituted with 1 oxo and additionally optionally substituted with 1, 2, or 3 R^{2a} groups;
each R²a is independently selected from cyano, nitro, halo, hydroxy, alkyl, alkenyl, carboxy, alkoxycarbonyl, alkylcarbonyl, alkylsulfinyl, alkylsulfonyl, alkylsulfonyloxyalkyl, alkoxyalkyl, alkoxyalkenyl, aminoalkyl, alkylaminoalkyl, dialkylaminoalkyl, hydroxyalkyl, cycloalkyl, cycloalkylalkyl, heterocycloalkyl, heterocycloalkylalkyl, 1,3-dioxo-isoindolinylalkyl, —NR^{2b}R^{2c}, and —OR^{2d}; where each heterocycloalkyl, either alone or as part of another group, is optionally substituted with 1, 2, 3, or 4 groups independently selected from alkyl, hydroxy, alkylcarbonyl, and alkoxycarbonyl;
R^{2b} is hydrogen or alkyl;
R^{2c} is hydrogen, alkyl, aminoalkyl, alkylaminoalkyl, dialkylaminoalkyl, alkoxyalkyl, hydroxyalkyl, alkylcarbonyl, cycloalkylcarbonyl, alkylsulfonyl, alkoxycarbonyl, cycloalkylalkyl, or heterocycloalkylalkyl; where the heterocycloalkyl in heterocycloalkylalkyl is optionally substituted with 1, 2, 3, or 4 groups independently selected from alkyl, hydroxy, alkylcarbonyl, and alkoxycarbonyl;
R^{2d} is alkyl, haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl; where each heterocycloalkyl, either alone or as part of another group, is optionally substituted with 1 or 2 groups independently selected from alkyl, hydroxy, alkylcarbonyl, and alkoxycarbonyl;
R⁴ is hydrogen, methyl, halo, or —CN;
each R⁵ is independently halo, hydroxy, alkoxycarbonyl, alkyl, haloalkyl, cycloalkyl, cycloalkylalkyl, heterocycloalkyl, heterocycloalkylalkyl, phenyl, or phenylmethyl which is optionally substituted with 1 or 2 alkoxy; and
R⁶ is halo, hydroxy, or alkoxy;
optionally a tautomer, a single stereoisomer or mixture of stereoisomers thereof and additionally optionally a pharmaceutically acceptable salt thereof;
provided that when R³ is pyrazolyl, then R^{3c} is not cyclopropyl.

In certain embodiments, the compound of Formula II is that wherein R¹ is morpholin-4-yl; and all other groups are as defined in the Summary of the Invention or in any of the embodiments.

In certain embodiments, the compound of Formula II is that wherein R² is indazolyl, pyrrolopyridinyl, benzimidazolyl, or imidazopyridinyl, each of which is optionally substituted with 1, 2, or 3 R^{2a} groups; and all other groups are as defined in the Summary of the Invention or in any of the embodiments.

In certain embodiments, the compound of Formula II is that wherein R⁴ is hydrogen or methyl; and all other groups are as defined in the Summary of the Invention or in any of the embodiments. In certain embodiments, the Compound of Formula II is that wherein R⁴ is hydrogen; and all other groups are as defined in the Summary of the Invention or in any of the embodiments.

Any combination of the groups described above for the various variables is contemplated herein.

In certain embodiments, provided in Table 1 are compounds of the following structures:

TABLE 1

| Cmpd | | |
|---|---|---|
| No. | Structure | Name |
| 1 | 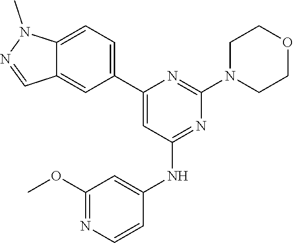 [SMILES: COc1cc(Nc2cc(-c3ccc4c(cnn4C)c3)nc(N3CCOCC3)n2)ccn1] | (2-methoxy(4-pyridyl))[6-(1- methyl(1H-indazol-5-yl))-2-morpholin- 4-ylpyrimidin-4-yl]amine |
| 2 | 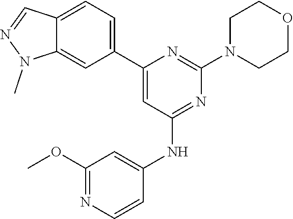 [SMILES: COc1cc(Nc2cc(-c3ccc4cnn(C)c4c3)nc(N3CCOCC3)n2)ccn1] | (2-methoxy(4-pyridyl))[6-(1- methyl(1H-indazol-6-yl))-2-morpholin- 4-ylpyrimidin-4-yl]amine |
| 3 | 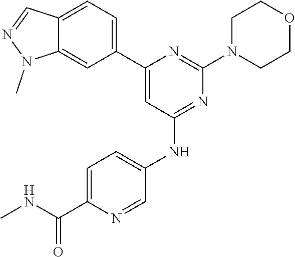 [SMILES: CNC(=O)c1ccc(Nc2cc(-c3ccc4cnn(C)c4c3)nc(N3CCOCC3)n2)cn1] | N-methyl(5-{[6-(1-methyl(1H-indazol- 6-yl))-2-morpholin-4-ylpyrimidin-4- yl]amino}(2-pyridyl))carboxamide |
| 4 | 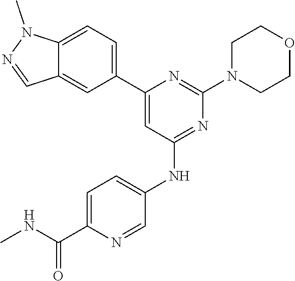 [SMILES: CNC(=O)c1ccc(Nc2cc(-c3ccc4c(cnn4C)c3)nc(N3CCOCC3)n2)cn1] | N-methyl(5-{[6-(1-methyl(1H-indazol- 5-yl))-2-morpholin-4-ylpyrimidin-4- yl]amino}(2-pyridyl))carboxamide |
| 5 | 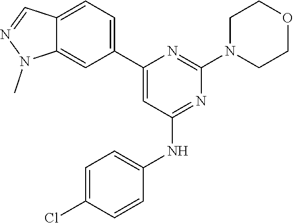 [SMILES: Cn1ncc2ccc(-c3cc(Nc4ccc(Cl)cc4)nc(N4CCOCC4)n3)cc21] | (4-chlorophenyl)[6-(1-methyl(1H- indazol-6-yl))-2-morpholin-4- ylpyrimidin-4-yl]amine |
| 6 | 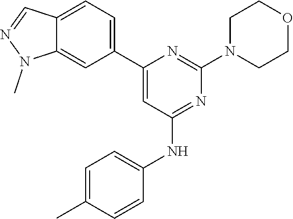 [SMILES: Cc1ccc(Nc2cc(-c3ccc4cnn(C)c4c3)nc(N3CCOCC3)n2)cc1] | [6-(1-methyl(1H-indazol-6-yl))-2- morpholin-4-ylpyrimidin-4-yl](4- methylphenyl)amine |
| 7 | 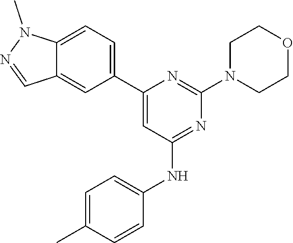 [SMILES: Cc1ccc(Nc2cc(-c3ccc4c(cnn4C)c3)nc(N3CCOCC3)n2)cc1] | [6-(1-methyl(1H-indazol-5-yl))-2- morpholin-4-ylpyrimidin-4-yl](4- methylphenyl)amine |
| 8 | 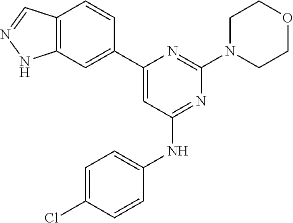 [SMILES: Clc1ccc(Nc2cc(-c3ccc4cn[nH]c4c3)nc(N3CCOCC3)n2)cc1] | (6-(1H-indazol-6-yl)-2-morpholin-4- ylpyrimidin-4-yl)(4-chlorophenyl)amine |
| 9 | 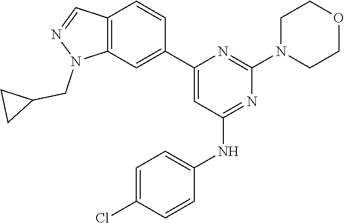 [SMILES: Clc1ccc(Nc2cc(-c3ccc4cnn(CC5CC5)c4c3)nc(N3CCOCC3)n2)cc1] | (4-chlorophenyl){6-[1- (cyclopropylmethyl)(1H-indazol-6-yl)]- 2-morpholin-4-ylpyrimidin-4-yl}amine |
| 10 | 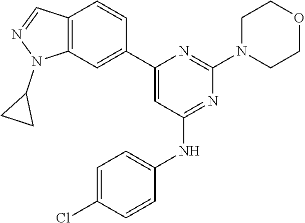 [SMILES: Clc1ccc(Nc2cc(-c3ccc4cnn(C5CC5)c4c3)nc(N3CCOCC3)n2)cc1] | (4-chlorophenyl)[6-(1-cyclopropyl(1H- indazol-6-yl))-2-morpholin-4- ylpyrimidin-4-yl]amine |
| 11 | 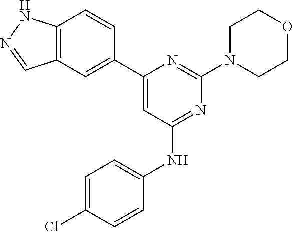 [SMILES: Clc1ccc(Nc2cc(-c3ccc4[nH]ncc4c3)nc(N3CCOCC3)n2)cc1] | (6-(1H-indazol-5-yl)-2-morpholin-4- ylpyrimidin-4-yl)(4-chlorophenyl)amine |
| 12 | 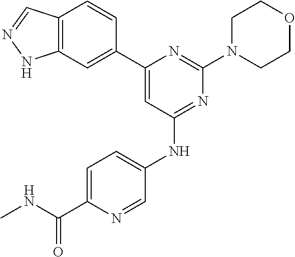 [SMILES: CNC(=O)c1ccc(Nc2cc(-c3ccc4cn[nH]c4c3)nc(N3CCOCC3)n2)cn1] | {5-[(6-(1H-indazol-6-yl)-2-morpholin- 4-ylpyrimidin-4-yl)amino](2-pyridyl)}- N-methylcarboxamide |
| 13 | 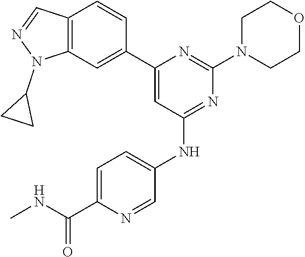 [SMILES: CNC(=O)c1ccc(Nc2cc(-c3ccc4cnn(C5CC5)c4c3)nc(N3CCOCC3)n2)cn1] | (5-{[6-(1-cyclopropyl(1H-indazol-6- yl))-2-morpholin-4-ylpyrimidin-4- yl]amino}(2-pyridyl))-N- methylcarboxamide |
| 14 | 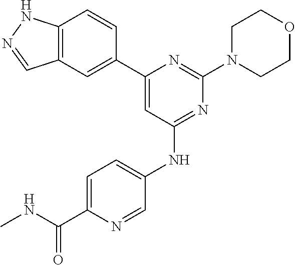 [SMILES: CNC(=O)c1ccc(Nc2cc(-c3ccc4[nH]ncc4c3)nc(N3CCOCC3)n2)cn1] | {5-[(6-(1H-indazol-5-yl)-2-morpholin- 4-ylpyrimidin-4-yl)amino](2-pyridyl)}- N-methylcarboxamide |
| 15 | 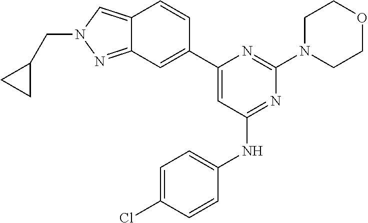 [SMILES: Clc1ccc(Nc2cc(-c3ccc4cn(CC5CC5)nc4c3)nc(N3CCOCC3)n2)cc1] | (4-chlorophenyl){6-[2- (cyclopropylmethyl)(2H-indazol-6-yl)]- 2-morpholin-4-ylpyrimidin-4-yl}amine |
| 16 | 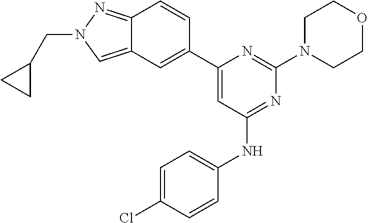 [SMILES: Clc1ccc(Nc2cc(-c3ccc4nn(CC5CC5)cc4c3)nc(N3CCOCC3)n2)cc1] | (4-chlorophenyl){6-[2- (cyclopropylmethyl)(2H-indazol-5-yl)]- 2-morpholin-4-ylpyrimidin-4-yl}amine |
| 17 | 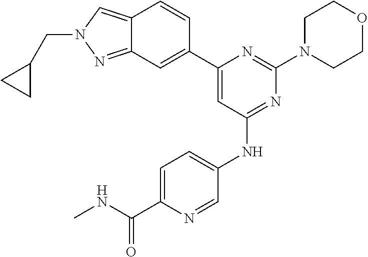 [SMILES: CNC(=O)c1ccc(Nc2cc(-c3ccc4cn(CC5CC5)nc4c3)nc(N3CCOCC3)n2)cn1] | [5-({6-[2-(cyclopropylmethyl)(2H- indazol-6-yl)]-2-morpholin-4- ylpyrimidin-4-yl}amino)(2-pyridyl)]-N- methylcarboxamide |
| 18 | 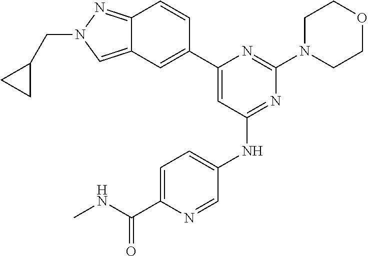 [SMILES: CNC(=O)c1ccc(Nc2cc(-c3ccc4nn(CC5CC5)cc4c3)nc(N3CCOCC3)n2)cn1] | [5-({6-[2-(cyclopropylmethyl)(2H- indazol-5-yl)]-2-morpholin-4- ylpyrimidin-4-yl}amino)(2-pyridyl)]-N- methylcarboxamide |
| 19 | 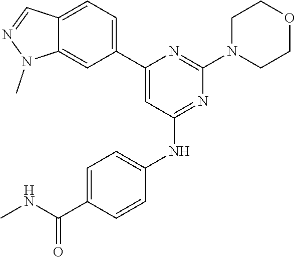 [SMILES: CNC(=O)c1ccc(Nc2cc(-c3ccc4cnn(C)c4c3)nc(N3CCOCC3)n2)cc1] | N-methyl(4-{[6-(1-methyl(1H-indazol- 6-yl))-2-morpholin-4-ylpyrimidin-4- yl]amino}phenyl)carboxamide |
| 20 | 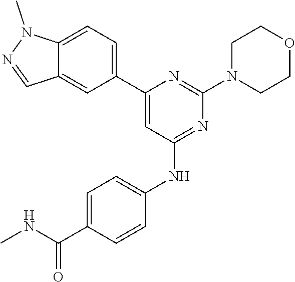 [SMILES: CNC(=O)c1ccc(Nc2cc(-c3ccc4c(cnn4C)c3)nc(N3CCOCC3)n2)cc1] | N-methyl(4-{[6-(1-methyl(1H-indazol- 5-yl))-2-morpholin-4-ylpyrimidin-4- yl]amino}phenyl)carboxamide |
| 21 | 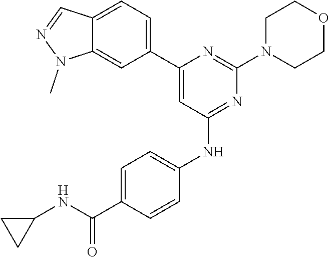 [SMILES: Cn1ncc2ccc(-c3cc(Nc4ccc(C(=O)NC5CC5)cc4)nc(N4CCOCC4)n3)cc21] | N-cyclopropyl(4-{[6-(1-methyl(1H- indazol-6-yl))-2-morpholin-4- ylpyrimidin-4- yl]amino}phenyl)carboxamide |
| 22 | 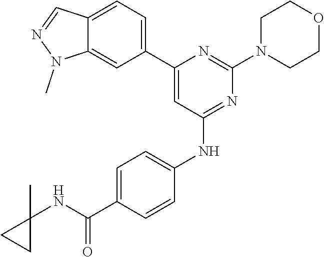 [SMILES: Cn1ncc2ccc(-c3cc(Nc4ccc(C(=O)NC5(C)CC5)cc4)nc(N4CCOCC4)n3)cc21] | (4-{[6-(1-methyl(1H-indazol-6-yl))-2- morpholin-4-ylpyrimidin-4- yl]amino}phenyl)-N- (methylcyclopropyl)carboxamide |
| 23 | 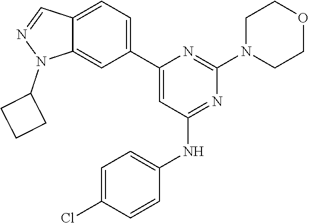 [SMILES: Clc1ccc(Nc2cc(-c3ccc4cnn(C5CCC5)c4c3)nc(N3CCOCC3)n2)cc1] | (4-chlorophenyl)[6-(1-cyclobutyl(1H- indazol-6-yl))-2-morpholin-4- ylpyrimidin-4-yl]amine |
| 24 | 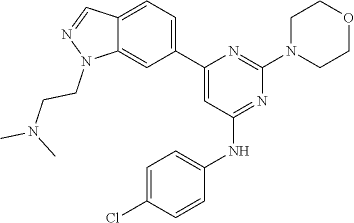 [SMILES: CN(C)CCn1ncc2ccc(-c3cc(Nc4ccc(Cl)cc4)nc(N4CCOCC4)n3)cc21] | [2-(6-{6-[(4-chlorophenyl)amino]-2- morpholin-4-ylpyrimidin-4-yl}(1H- indazolyl))ethyl]dimethylamine |
| 25 | 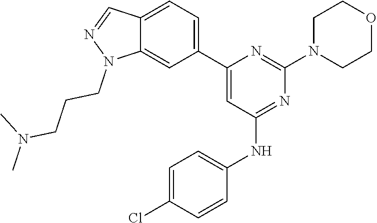 [SMILES: CN(C)CCCn1ncc2ccc(-c3cc(Nc4ccc(Cl)cc4)nc(N4CCOCC4)n3)cc21] | [3-(6-{6-[(4-chlorophenyl)amino]-2- morpholin-4-ylpyrimidin-4-yl}(1H- indazolyl))propyl]dimethylamine |
| 26 | 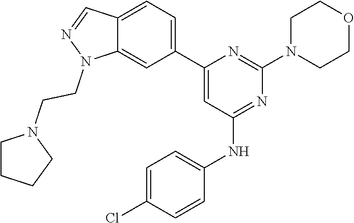 [SMILES: Clc1ccc(Nc2cc(-c3ccc4cnn(CCN5CCCC5)c4c3)nc(N3CCOCC3)n2)cc1] | (4-chlorophenyl){2-morpholin-4-yl-6- [1-(2-pyrrolidinylethyl)(1H-indazol-6- yl)]pyrimidin-4-yl}amine |
| 27 | 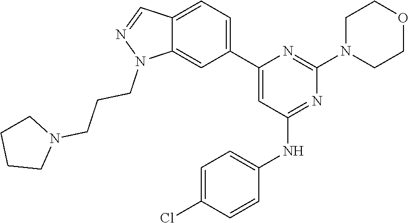 [SMILES: Clc1ccc(Nc2cc(-c3ccc4cnn(CCCN5CCCC5)c4c3)nc(N3CCOCC3)n2)cc1] | (4-chlorophenyl){2-morpholin-4-yl-6- [1-(3-pyrrolidinylpropyl)(1H-indazol-6- yl)]pyrimidin-4-yl}amine |
| 28 | 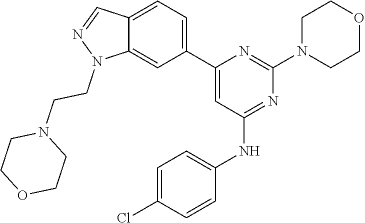 [SMILES: Clc1ccc(Nc2cc(-c3ccc4cnn(CCN5CCOCC5)c4c3)nc(N3CCOCC3)n2)cc1] | (4-chlorophenyl){2-morpholin-4-yl-6- [1-(2-morpholin-4-ylethyl)(1H-indazol- 6-yl)]pyrimidin-4-yl}amine |
| 29 | 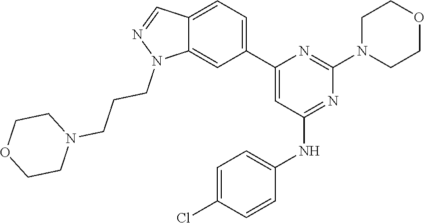 [SMILES: Clc1ccc(Nc2cc(-c3ccc4cnn(CCCN5CCOCC5)c4c3)nc(N3CCOCC3)n2)cc1] | (4-chlorophenyl){2-morpholin-4-yl-6- [1-(3-morpholin-4-ylpropyl)(1H- indazol-6-yl)]pyrimidin-4-yl}amine |
| 30 | 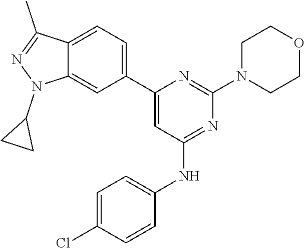 [SMILES: Cc1nn(C2CC2)c2cc(-c3cc(Nc4ccc(Cl)cc4)nc(N4CCOCC4)n3)ccc12] | (4-chlorophenyl)[6-(1-cyclopropyl-3- methyl(1H-indazol-6-yl))-2-morpholin- 4-ylpyrimidin-4-yl]amine |
| 31 | 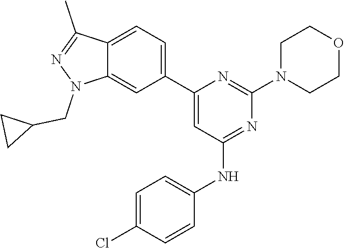 [SMILES: Cc1nn(CC2CC2)c2cc(-c3cc(Nc4ccc(Cl)cc4)nc(N4CCOCC4)n3)ccc12] | (4-chlorophenyl){6-[1- (cyclopropylmethyl)-3-methyl(1H- indazol-6-yl)]-2-morpholin-4- ylpyrimidin-4-yl}amine |
| 32 | 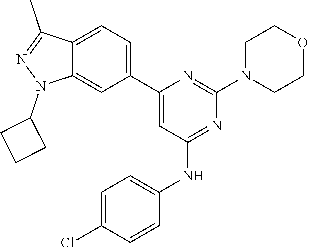 [SMILES: Cc1nn(C2CCC2)c2cc(-c3cc(Nc4ccc(Cl)cc4)nc(N4CCOCC4)n3)ccc12] | (4-chlorophenyl)[6-(1-cyclobutyl-3- methyl(1H-indazol-6-yl))-2-morpholin- 4-ylpyrimidin-4-yl]amine |
| 33 | 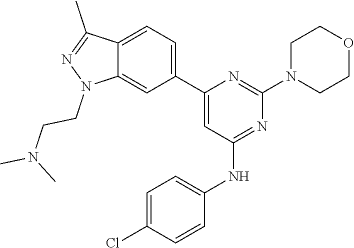 [SMILES: Cc1nn(CCN(C)C)c2cc(-c3cc(Nc4ccc(Cl)cc4)nc(N4CCOCC4)n3)ccc12] | [2-(6-{6-[(4-chlorophenyl)amino]-2- morpholin-4-ylpyrimidin-4-yl}-3- methyl(1H- indazolyl))ethyl]dimethylamine |
| 34 | 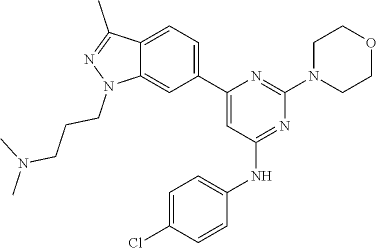 [SMILES: Cc1nn(CCCN(C)C)c2cc(-c3cc(Nc4ccc(Cl)cc4)nc(N4CCOCC4)n3)ccc12] | [3-(6-{6-[(4-chlorophenyl)amino]-2- morpholin-4-ylpyrimidin-4-yl}-3- methyl(1H- indazolyl))propyl]dimethylamine |
| 35 | 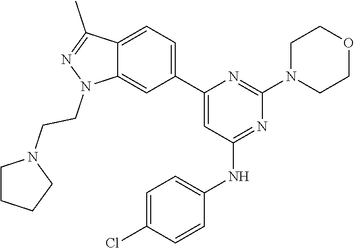 [SMILES: Cc1nn(CCN2CCCC2)c2cc(-c3cc(Nc4ccc(Cl)cc4)nc(N4CCOCC4)n3)ccc12] | (4-chlorophenyl){6-[3-methyl-1-(2- pyrrolidinylethyl)(1H-indazol-6-yl)]-2- morpholin-4-ylpyrimidin-4-yl}amine |
| 36 | 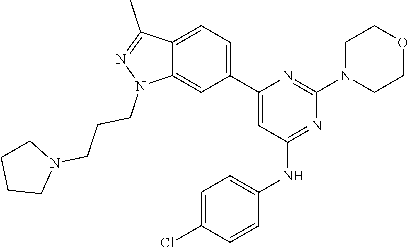 [SMILES: Cc1nn(CCCN2CCCC2)c2cc(-c3cc(Nc4ccc(Cl)cc4)nc(N4CCOCC4)n3)ccc12] | (4-chlorophenyl){6-[3-methyl-1-(3- pyrrolidinylpropyl)(1H-indazol-6-yl)]-2- morpholin-4-ylpyrimidin-4-yl}amine |
| 37 | 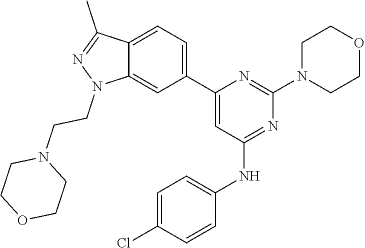 [SMILES: Cc1nn(CCN2CCOCC2)c2cc(-c3cc(Nc4ccc(Cl)cc4)nc(N4CCOCC4)n3)ccc12] | (4-chlorophenyl){6-[3-methyl-1-(2- morpholin-4-ylethyl)(1H-indazol-6-yl)]- 2-morpholin-4-ylpyrimidin-4-yl}amine |
| 38 | 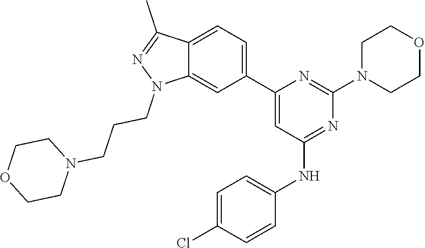 [SMILES: Cc1nn(CCCN2CCOCC2)c2cc(-c3cc(Nc4ccc(Cl)cc4)nc(N4CCOCC4)n3)ccc12] | (4-chlorophenyl){6-[3-methyl-1-(3- morpholin-4-ylpropyl)(1H-indazol-6- yl)]-2-morpholin-4-ylpyrimidin-4- yl}amine |
| 39 | 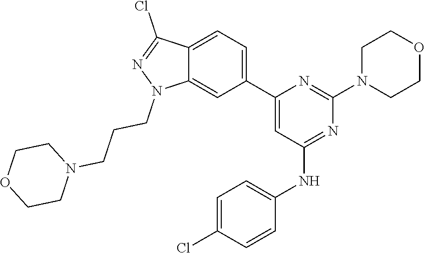 [SMILES: Clc1ccc(Nc2cc(-c3ccc4c(Cl)nn(CCCN5CCOCC5)c4c3)nc(N3CCOCC3)n2)cc1] | {6-[3-chloro-1-(3-morpholin-4- ylpropyl)(1H-indazol-6-yl)]-2- morpholin-4-ylpyrimidin-4-yl}(4- chlorophenyl)amine |
| 40 | 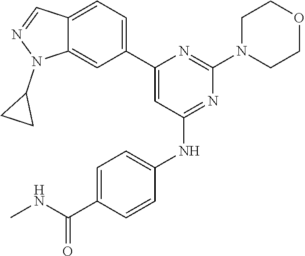 [SMILES: CNC(=O)c1ccc(Nc2cc(-c3ccc4cnn(C5CC5)c4c3)nc(N3CCOCC3)n2)cc1] | (4-{[6-(1-cyclopropyl(1H-indazol-6- yl))-2-morpholin-4-ylpyrimidin-4- yl]amino}phenyl)-N- methylcarboxamide |
| 41 | 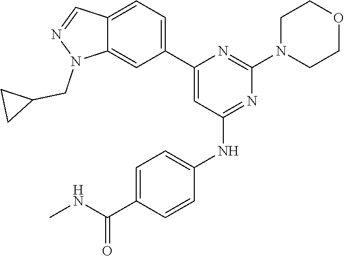 [SMILES: CNC(=O)c1ccc(Nc2cc(-c3ccc4cnn(CC5CC5)c4c3)nc(N3CCOCC3)n2)cc1] | [4-({6-[1-(cyclopropylmethyl)(1H- indazol-6-yl)]-2-morpholin-4- ylpyrimidin-4-yl}amino)phenyl]-N- methylcarboxamide |
| 42 | 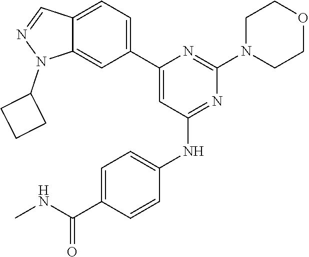 [SMILES: CNC(=O)c1ccc(Nc2cc(-c3ccc4cnn(C5CCC5)c4c3)nc(N3CCOCC3)n2)cc1] | (4-{[6-(1-cyclobutyl(1H-indazol-6-yl))- 2-morpholin-4-ylpyrimidin-4- yl]amino}phenyl)-N- methylcarboxamide |
| 43 | 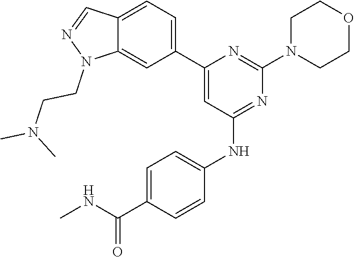 [SMILES: CNC(=O)c1ccc(Nc2cc(-c3ccc4cnn(CCN(C)C)c4c3)nc(N3CCOCC3)n2)cc1] | {4-[(6-{1-[2-(dimethylamino)ethyl](1H- indazol-6-yl)}-2-morpholin-4- ylpyrimidin-4-yl)amino]phenyl}-N- methylcarboxamide |
| 44 | 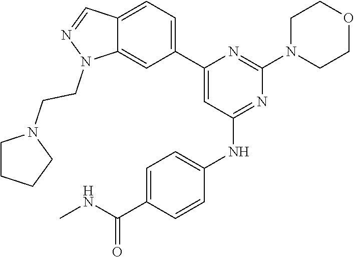 [SMILES: CNC(=O)c1ccc(Nc2cc(-c3ccc4cnn(CCN5CCCC5)c4c3)nc(N3CCOCC3)n2)cc1] | N-methyl[4-({2-morpholin-4-yl-6-[1-(2- pyrrolidinylethyl)(1H-indazol-6- yl)]pyrimidin-4- yl}amino)phenyl]carboxamide |
| 45 | 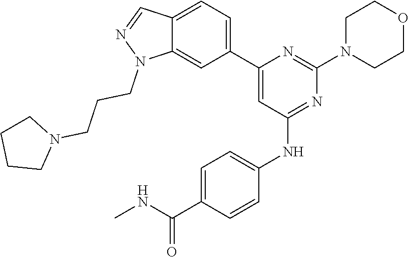 [SMILES: CNC(=O)c1ccc(Nc2cc(-c3ccc4cnn(CCCN5CCCC5)c4c3)nc(N3CCOCC3)n2)cc1] | N-methyl[4-({2-morpholin-4-yl-6-[1-(3- pyrrolidinylpropyl)(1H-indazol-6- yl)]pyrimidin-4- yl}amino)phenyl]carboxamide |
| 46 | 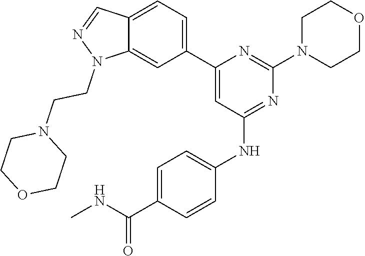 [SMILES: CNC(=O)c1ccc(Nc2cc(-c3ccc4cnn(CCN5CCOCC5)c4c3)nc(N3CCOCC3)n2)cc1] | N-methyl[4-({2-morpholin-4-yl-6-[1-(2- morpholin-4-ylethyl)(1H-indazol-6- yl)]pyrimidin-4- yl}amino)phenyl]carboxamide |
| 47 | 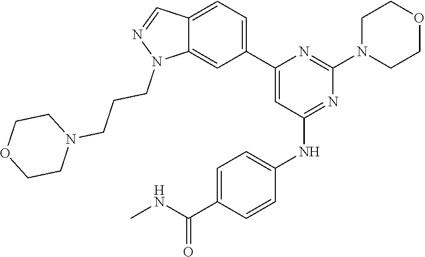 [SMILES: CNC(=O)c1ccc(Nc2cc(-c3ccc4cnn(CCCN5CCOCC5)c4c3)nc(N3CCOCC3)n2)cc1] | N-methyl[4-({2-morpholin-4-yl-6-[1-(3- morpholin-4-ylpropyl)(1H-indazol-6- yl)]pyrimidin-4- yl}amino)phenyl]carboxamide |
| 48 | 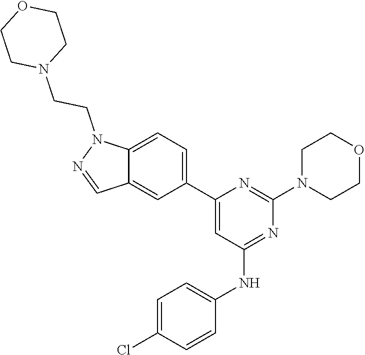 [SMILES: Clc1ccc(Nc2cc(-c3ccc4c(cnn4CCN4CCOCC4)c3)nc(N3CCOCC3)n2)cc1] | (4-chlorophenyl){2-morpholin-4-yl-6- [1-(2-morpholin-4-ylethyl)(1H-indazol- 5-yl)]pyrimidin-4-yl}amine |
| 49 | 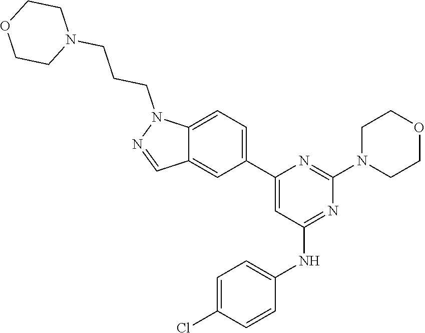 [SMILES: Clc1ccc(Nc2cc(-c3ccc4c(cnn4CCCN4CCOCC4)c3)nc(N3CCOCC3)n2)cc1] | (4-chlorophenyl){2-morpholin-4-yl-6- [1-(3-morpholin-4-ylpropyl)(1H- indazol-5-yl)]pyrimidin-4-yl}amine |
| 50 | 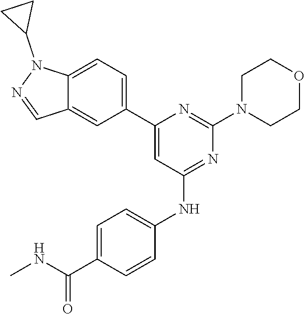 [SMILES: CNC(=O)c1ccc(Nc2cc(-c3ccc4c(cnn4C4CC4)c3)nc(N3CCOCC3)n2)cc1] | (4-{[6-(1-cyclopropyl(1H-indazol-5- yl))-2-morpholin-4-ylpyrimidin-4- yl]amino}phenyl)-N- methylcarboxamide |
| 51 | 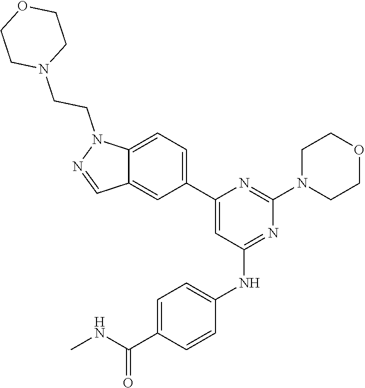 [SMILES: CNC(=O)c1ccc(Nc2cc(-c3ccc4c(cnn4CCN4CCOCC4)c3)nc(N3CCOCC3)n2)cc1] | N-methyl[4-({2-morpholin-4-yl-6-[1-(2- morpholin-4-ylethyl)(1H-indazol-5- yl)]pyrimidin-4- yl}amino)phenyl]carboxamide |
| 52 | 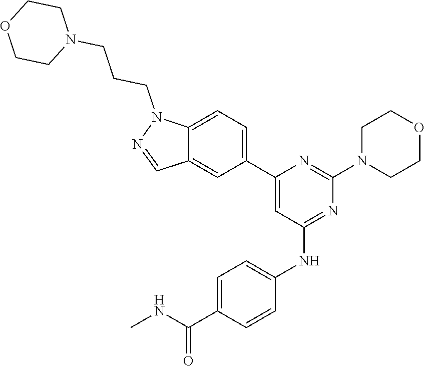 [SMILES: CNC(=O)c1ccc(Nc2cc(-c3ccc4c(cnn4CCCN4CCOCC4)c3)nc(N3CCOCC3)n2)cc1] | N-methyl[4-({2-morpholin-4-yl-6-[1-(3- morpholin-4-ylpropyl)(1H-indazol-5- yl)]pyrimidin-4- yl}amino)phenyl]carboxamide |
| 53 | 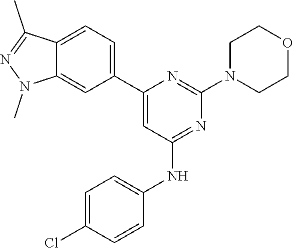 [SMILES: Cc1nn(C)c2cc(-c3cc(Nc4ccc(Cl)cc4)nc(N4CCOCC4)n3)ccc12] | [6-(1,3-dimethyl(1H-indazol-6-yl))-2- morpholin-4-ylpyrimidin-4-yl](4- chlorophenyl)amine |
| 54 | 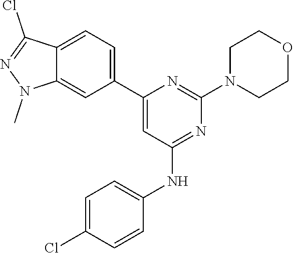 [SMILES: Cn1nc(Cl)c2ccc(-c3cc(Nc4ccc(Cl)cc4)nc(N4CCOCC4)n3)cc21] | [6-(3-chloro-1-methyl(1H-indazol-6- yl))-2-morpholin-4-ylpyrimidin-4-yl](4- chlorophenyl)amine |
| 55 | 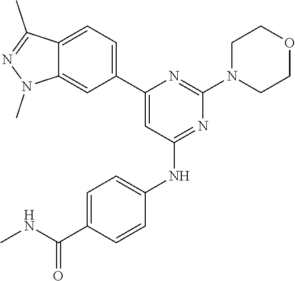 [SMILES: CNC(=O)c1ccc(Nc2cc(-c3ccc4c(C)nn(C)c4c3)nc(N3CCOCC3)n2)cc1] | (4-{[6-(1,3-dimethyl(1H-indazol-6-yl))- 2-morpholin-4-ylpyrimidin-4- yl]amino}phenyl)-N- methylcarboxamide |
| 56 | 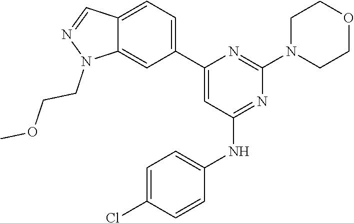 [SMILES: COCCn1ncc2ccc(-c3cc(Nc4ccc(Cl)cc4)nc(N4CCOCC4)n3)cc21] | (4-chlorophenyl){6-[1-(2- methoxyethyl)(1H-indazol-6-yl)]-2- morpholin-4-ylpyrimidin-4-yl}amine |
| 57 | 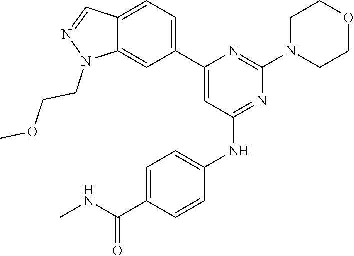 [SMILES: CNC(=O)c1ccc(Nc2cc(-c3ccc4cnn(CCOC)c4c3)nc(N3CCOCC3)n2)cc1] | [4-({6-[1-(2-methoxyethyl)(1H-indazol- 6-yl)]-2-morpholin-4-ylpyrimidin-4- yl}amino)phenyl]-N- methylcarboxamide |
| 58 | 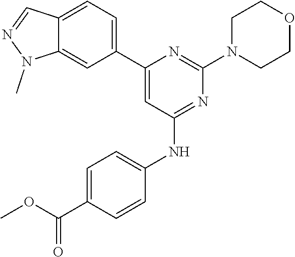 [SMILES: COC(=O)c1ccc(Nc2cc(-c3ccc4cnn(C)c4c3)nc(N3CCOCC3)n2)cc1] | methyl 4-{[6-(1-methyl(1H-indazol-6- yl))-2-morpholin-4-ylpyrimidin-4- yl]amino}benzoate |
| 59 | 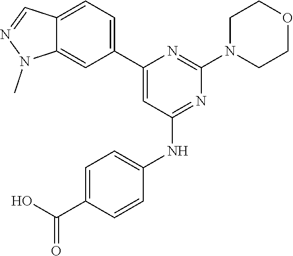 [SMILES: Cn1ncc2ccc(-c3cc(Nc4ccc(C(=O)O)cc4)nc(N4CCOCC4)n3)cc21] | 4-{[6-(1-methyl(1H-indazol-6-yl))-2- morpholin-4-ylpyrimidin-4- yl]amino}benzoic acid |
| 60 | 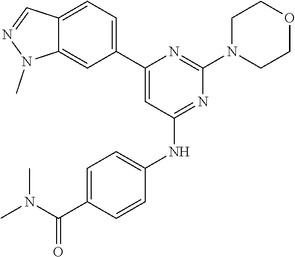 [SMILES: CN(C)C(=O)c1ccc(Nc2cc(-c3ccc4cnn(C)c4c3)nc(N3CCOCC3)n2)cc1] | N,N-dimethyl(4-{[6-(1-methyl(1H- indazol-6-yl))-2-morpholin-4- ylpyrimidin-4- yl]amino}phenyl)carboxamide |
| 61 | 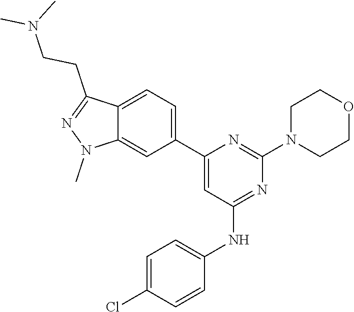 [SMILES: CN(C)CCc1nn(C)c2cc(-c3cc(Nc4ccc(Cl)cc4)nc(N4CCOCC4)n3)ccc12] | [2-(6-{6-[(4-chlorophenyl)amino]-2- morpholin-4-ylpyrimidin-4-yl}-1- methyl(1H-indazol-3- yl))ethyl]dimethylamine |
| 62 | 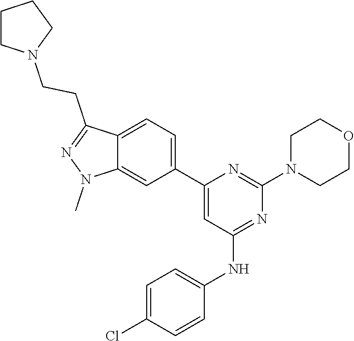 [SMILES: Cn1nc(CCN2CCCC2)c2ccc(-c3cc(Nc4ccc(Cl)cc4)nc(N4CCOCC4)n3)cc21] | (4-chlorophenyl){6-[1-methyl-3-(2- pyrrolidinylethyl)(1H-indazol-6-yl)]-2- morpholin-4-ylpyrimidin-4-yl}amine |
| 63 | 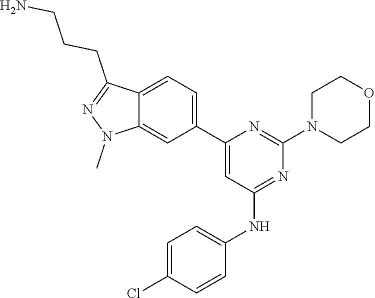 [SMILES: Cn1nc(CCCN)c2ccc(-c3cc(Nc4ccc(Cl)cc4)nc(N4CCOCC4)n3)cc21] | {6-[3-(3-aminopropyl)-1-methyl(1H- indazol-6-yl)]-2-morpholin-4- ylpyrimidin-4-yl}(4- chlorophenyl)amine |
| 64 | 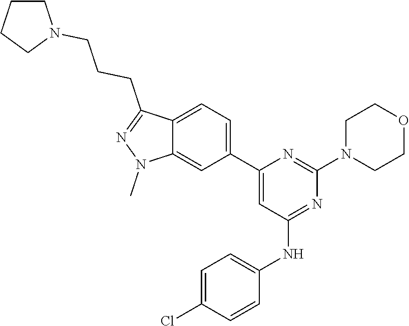 [SMILES: Cn1nc(CCCN2CCCC2)c2ccc(-c3cc(Nc4ccc(Cl)cc4)nc(N4CCOCC4)n3)cc21] | (4-chlorophenyl){6-[1-methyl-3-(3- pyrrolidinylpropyl)(1H-indazol-6-yl)]-2- morpholin-4-ylpyrimidin-4-yl}amine |
| 65 | 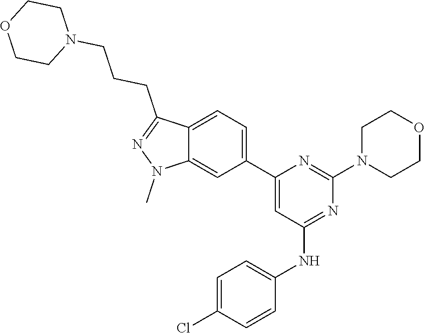 [SMILES: Cn1nc(CCCN2CCOCC2)c2ccc(-c3cc(Nc4ccc(Cl)cc4)nc(N4CCOCC4)n3)cc21] | (4-chlorophenyl){6-[1-methyl-3-(3- morpholin-4-ylpropyl)(1H-indazol-6- yl)]-2-morpholin-4-ylpyrimidin-4- yl}amine |
| 66 | 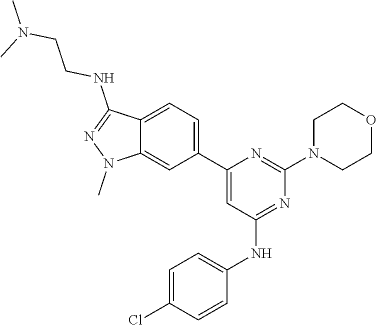 [SMILES: CN(C)CCNc1nn(C)c2cc(-c3cc(Nc4ccc(Cl)cc4)nc(N4CCOCC4)n3)ccc12] | {2-[(6-{6-[(4-chlorophenyl)amino]-2- morpholin-4-ylpyrimidin-4-yl}-1- methyl(1H-indazol-3- yl))amino]ethyl}dimethylamine |
| 67 | 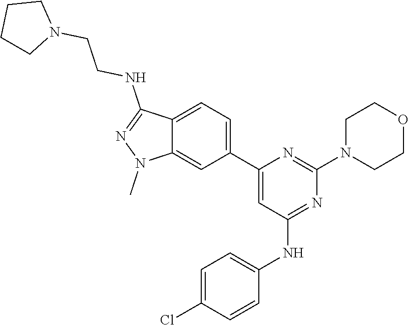 [SMILES: Cn1nc(NCCN2CCCC2)c2ccc(-c3cc(Nc4ccc(Cl)cc4)nc(N4CCOCC4)n3)cc21] | (4-chlorophenyl)(6-{1-methyl-3-[(2- pyrrolidinylethyl)amino](1H-indazol-6- yl)}-2-morpholin-4-ylpyrimidin-4- yl)amine |
| 68 | 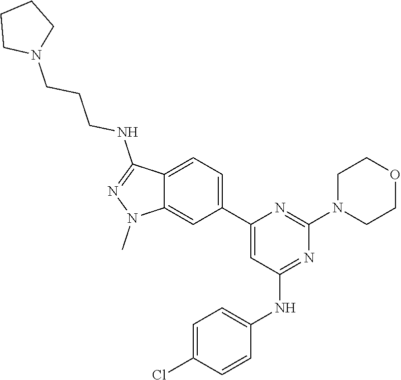 [SMILES: Cn1nc(NCCCN2CCCC2)c2ccc(-c3cc(Nc4ccc(Cl)cc4)nc(N4CCOCC4)n3)cc21] | (4-chlorophenyl)(6-{1-methyl-3-[(3- pyrrolidinylpropyl)amino](1H-indazol- 6-yl)}-2-morpholin-4-ylpyrimidin-4- yl)amine |
| 69 | 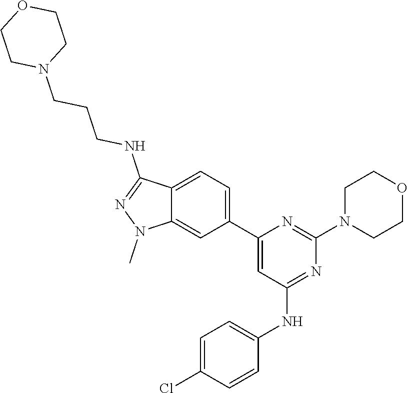 [SMILES: Cn1nc(NCCCN2CCOCC2)c2ccc(-c3cc(Nc4ccc(Cl)cc4)nc(N4CCOCC4)n3)cc21] | (4-chlorophenyl)(6-{1-methyl-3-[(3- morpholin-4-ylpropyl)amino](1H- indazol-6-yl)}-2-morpholin-4- ylpyrimidin-4-yl)amine |
| 70 | 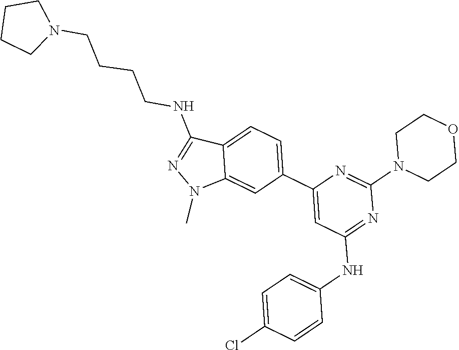 [SMILES: Cn1nc(NCCCCN2CCCC2)c2ccc(-c3cc(Nc4ccc(Cl)cc4)nc(N4CCOCC4)n3)cc21] | (4-chlorophenyl)(6-{1-methyl-3-[(4- pyrrolidinylbutyl)amino](1H-indazol-6- yl)}-2-morpholin-4-ylpyrimidin-4- yl)amine |
| 71 | 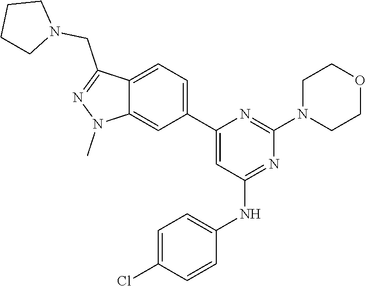 [SMILES: Cn1nc(CN2CCCC2)c2ccc(-c3cc(Nc4ccc(Cl)cc4)nc(N4CCOCC4)n3)cc21] | (4-chlorophenyl){6-[1-methyl-3- (pyrrolidinylmethyl)(1H-indazol-6-yl)]- 2-morpholin-4-ylpyrimidin-4-yl}amine |
| 72 | 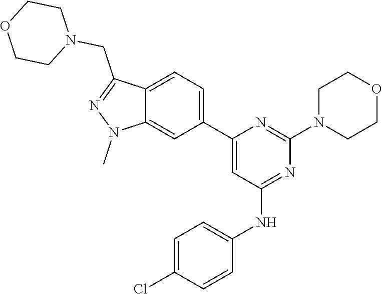 [SMILES: Cn1nc(CN2CCOCC2)c2ccc(-c3cc(Nc4ccc(Cl)cc4)nc(N4CCOCC4)n3)cc21] | (4-chlorophenyl){6-[1-methyl-3- (morpholin-4-ylmethyl)(1H-indazol-6- yl)]-2-morpholin-4-ylpyrimidin-4- yl}amine |
| 73 | 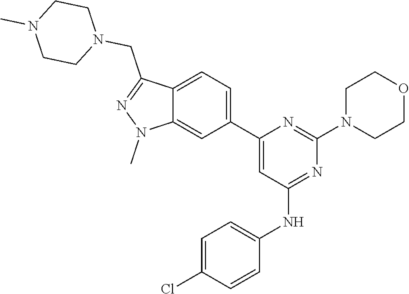 [SMILES: CN1CCN(Cc2nn(C)c3cc(-c4cc(Nc5ccc(Cl)cc5)nc(N5CCOCC5)n4)ccc23)CC1] | (4-chlorophenyl)(6-{1-methyl-3-[(4- methylpiperazinyl)methyl](1H-indazol- 6-yl)}-2-morpholin-4-ylpyrimidin-4- yl)amine |
| 74 | 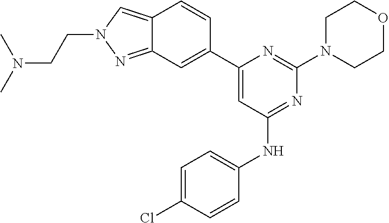 [SMILES: CN(C)CCn1cc2ccc(-c3cc(Nc4ccc(Cl)cc4)nc(N4CCOCC4)n3)cc2n1] | [2-(6-{6-[(4-chlorophenyl)amino]-2- morpholin-4-ylpyrimidin-4-yl}(2H- indazol-2-yl))ethyl]dimethylamine |
| 75 | 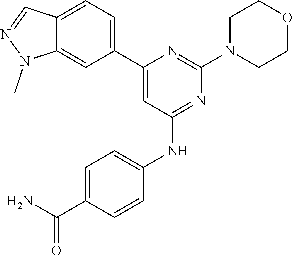 [SMILES: Cn1ncc2ccc(-c3cc(Nc4ccc(C(N)=O)cc4)nc(N4CCOCC4)n3)cc21] | 4-{[6-(1-methyl(1H-indazol-6-yl))-2- morpholin-4-ylpyrimidin-4- yl]amino}benzamide |
| 76 | 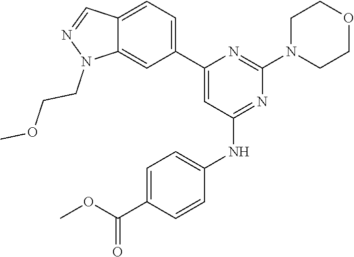 [SMILES: COCCn1ncc2ccc(-c3cc(Nc4ccc(C(=O)OC)cc4)nc(N4CCOCC4)n3)cc21] | methyl 4-({6-[1-(2-methoxyethyl)(1H- indazol-6-yl)]-2-morpholin-4- ylpyrimidin-4-yl}amino)benzoate |
| 77 | 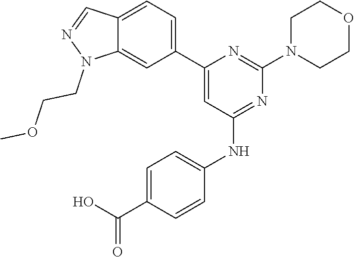 [SMILES: COCCn1ncc2ccc(-c3cc(Nc4ccc(C(=O)O)cc4)nc(N4CCOCC4)n3)cc21] | 4-({6-[1-(2-methoxyethyl)(1H-indazol- 6-yl)]-2-morpholin-4-ylpyrimidin-4- yl}amino)benzoic acid |
| 78 | 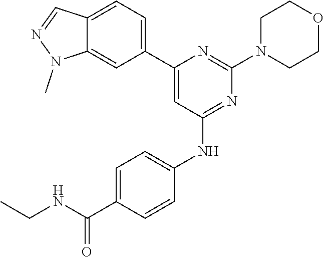 [SMILES: CCNC(=O)c1ccc(Nc2cc(-c3ccc4cnn(C)c4c3)nc(N3CCOCC3)n2)cc1] | N-ethyl(4-{[6-(1-methyl(1H-indazol-6- yl))-2-morpholin-4-ylpyrimidin-4- yl]amino}phenyl)carboxamide |
| 79 | 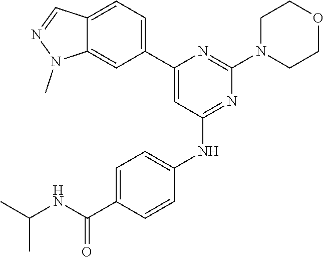 [SMILES: CC(C)NC(=O)c1ccc(Nc2cc(-c3ccc4cnn(C)c4c3)nc(N3CCOCC3)n2)cc1] | (4-{[6-(1-methyl(1H-indazol-6-yl))-2- morpholin-4-ylpyrimidin-4- yl]amino}phenyl)-N- (methylethyl)carboxamide |
| 80 | 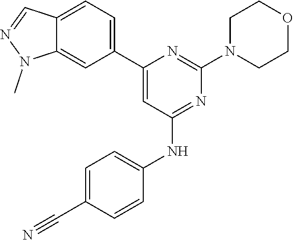 [SMILES: Cn1ncc2ccc(-c3cc(Nc4ccc(C#N)cc4)nc(N4CCOCC4)n3)cc21] | 4-{[6-(1-methyl(1H-indazol-6-yl))-2- morpholin-4-ylpyrimidin-4- yl]amino}benzenecarbonitrile |
| 81 | 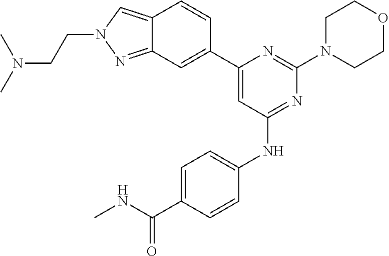 [SMILES: CNC(=O)c1ccc(Nc2cc(-c3ccc4cn(CCN(C)C)nc4c3)nc(N3CCOCC3)n2)cc1] | {4-[(6-{2-[2-(dimethylamino)ethyl](2H- indazol-6-yl)}-2-morpholin-4- ylpyrimidin-4-yl)amino]phenyl}-N- methylcarboxamide |
| 82 | 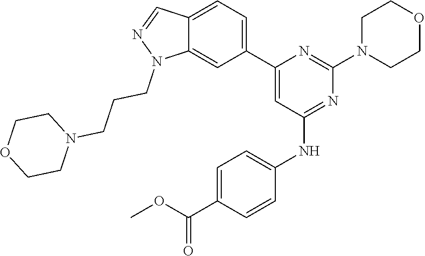 [SMILES: COC(=O)c1ccc(Nc2cc(-c3ccc4cnn(CCCN5CCOCC5)c4c3)nc(N3CCOCC3)n2)cc1] | methyl 4-({2-morpholin-4-yl-6-[1-(3- morpholin-4-ylpropyl)(1H-indazol-6- yl)]pyrimidin-4-yl}amino)benzoate |
| 83 | 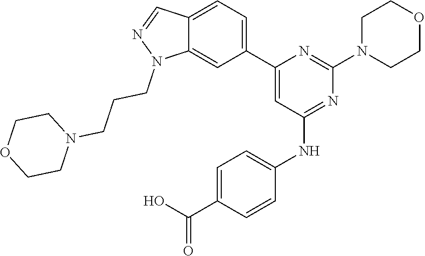 [SMILES: O=C(O)c1ccc(Nc2cc(-c3ccc4cnn(CCCN5CCOCC5)c4c3)nc(N3CCOCC3)n2)cc1] | 4-({2-morpholin-4-yl-6-[1-(3- morpholin-4-ylpropyl)(1H-indazol-6- yl)]pyrimidin-4-yl}amino)benzoic acid |
| 84 | 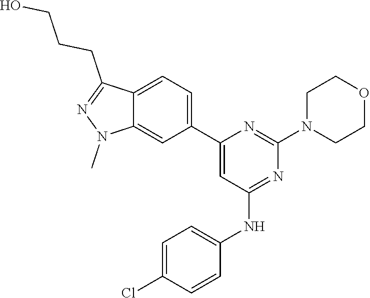 [SMILES: Cn1nc(CCCO)c2ccc(-c3cc(Nc4ccc(Cl)cc4)nc(N4CCOCC4)n3)cc21] | 3-(6-{6-[(4-chlorophenyl)amino]-2- morpholin-4-ylpyrimidin-4-yl}-1- methyl-1H-indazol-3-yl)propan-1-ol |
| 85 | 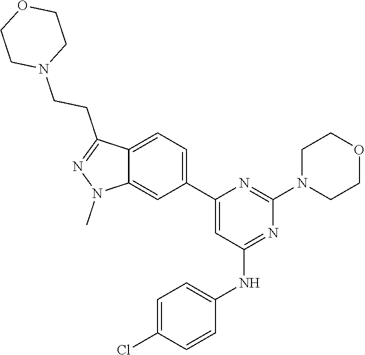 [SMILES: Cn1nc(CCN2CCOCC2)c2ccc(-c3cc(Nc4ccc(Cl)cc4)nc(N4CCOCC4)n3)cc21] | (4-chlorophenyl){6-[1-methyl-3-(2- morpholin-4-ylethyl)(1H-indazol-6-yl)]- 2-morpholin-4-ylpyrimidin-4-yl}amine |
| 86 | 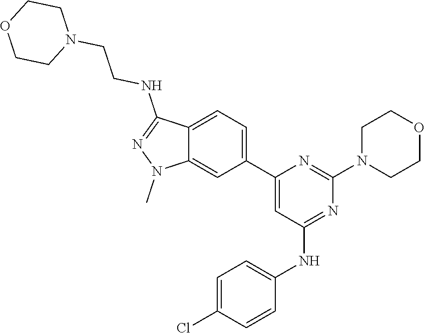 [SMILES: Cn1nc(NCCN2CCOCC2)c2ccc(-c3cc(Nc4ccc(Cl)cc4)nc(N4CCOCC4)n3)cc21] | (4-chlorophenyl)(6-{1-methyl-3-[(2- morpholin-4-ylethyl)amino](1H- indazol-6-yl)}-2-morpholin-4- ylpyrimidin-4-yl)amine |
| 87 | 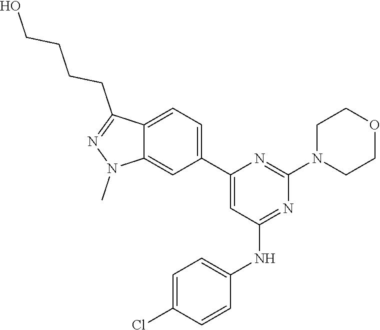 [SMILES: Cn1nc(CCCCO)c2ccc(-c3cc(Nc4ccc(Cl)cc4)nc(N4CCOCC4)n3)cc21] | 4-(6-{6-[(4-chlorophenyl)amino]-2- morpholin-4-ylpyrimidin-4-yl}-1- methyl-1H-indazol-3-yl)butan-1-ol |
| 88 | 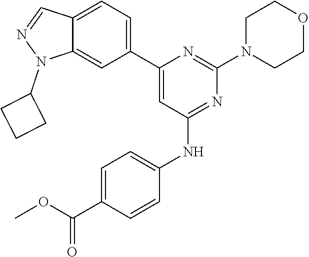 [SMILES: COC(=O)c1ccc(Nc2cc(-c3ccc4cnn(C5CCC5)c4c3)nc(N3CCOCC3)n2)cc1] | methyl 4-{[6-(1-cyclobutyl(1H-indazol- 6-yl))-2-morpholin-4-ylpyrimidin-4- yl]amino}benzoate |
| 89 | 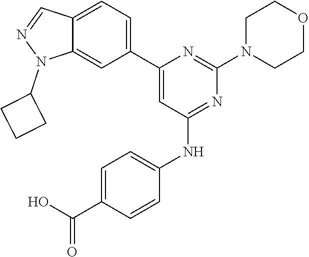 [SMILES: O=C(O)c1ccc(Nc2cc(-c3ccc4cnn(C5CCC5)c4c3)nc(N3CCOCC3)n2)cc1] | 4-{[6-(1-cyclobutyl(1H-indazol-6-yl))- 2-morpholin-4-ylpyrimidin-4- yl]amino}benzoic acid |
| 90 | 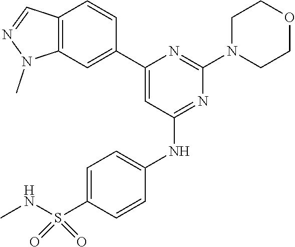 [SMILES: CNS(=O)(=O)c1ccc(Nc2cc(-c3ccc4cnn(C)c4c3)nc(N3CCOCC3)n2)cc1] | methyl[(4-{[6-(1-methyl(1H-indazol-6- yl))-2-morpholin-4-ylpyrimidin-4- yl]amino}phenyl)sulfonyl]amine |
| 91 | 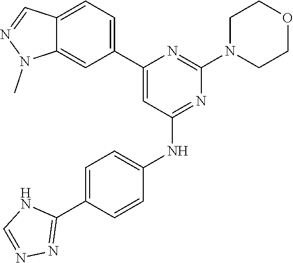 [SMILES: Cn1ncc2ccc(-c3cc(Nc4ccc(-c5nnc[nH]5)cc4)nc(N4CCOCC4)n3)cc21] | (4-(4H-1,2,4-triazol-3-yl)phenyl)[6-(1- methyl(1H-indazol-6-yl))-2-morpholin- 4-ylpyrimidin-4-yl]amine |
| 92 | 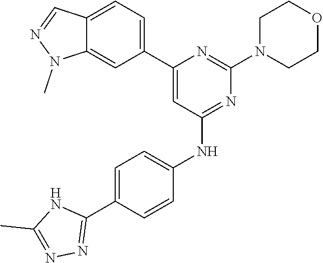 [SMILES: Cc1nnc(-c2ccc(Nc3cc(-c4ccc5cnn(C)c5c4)nc(N4CCOCC4)n3)cc2)[nH]1] | [6-(1-methyl(1H-indazol-6-yl))-2- morpholin-4-ylpyrimidin-4-yl][4-(5- methyl(4H-1,2,4-triazol-3- yl))phenyl]amine |
| 93 | 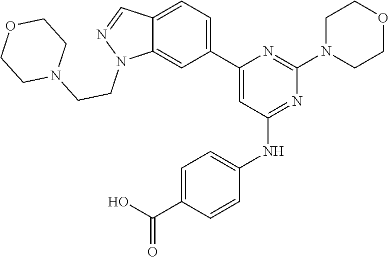 [SMILES: O=C(O)c1ccc(Nc2cc(-c3ccc4cnn(CCN5CCOCC5)c4c3)nc(N3CCOCC3)n2)cc1] | 4-({2-morpholin-4-yl-6-[1-(2- morpholin-4-ylethyl)(1H-indazol-6- yl)]pyrimidin-4-yl}amino)benzoic acid |
| 94 | 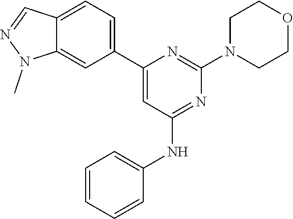 [SMILES: Cn1ncc2ccc(-c3cc(Nc4ccccc4)nc(N4CCOCC4)n3)cc21] | [6-(1-methyl(1H-indazol-6-yl))-2- morpholin-4-ylpyrimidin-4- yl]phenylamine |
| 95 | 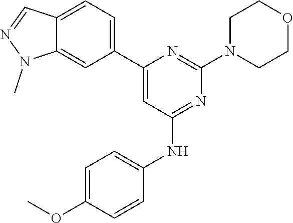 [SMILES: COc1ccc(Nc2cc(-c3ccc4cnn(C)c4c3)nc(N3CCOCC3)n2)cc1] | (4-methoxyphenyl)[6-(1-methyl(1H- indazol-6-yl))-2-morpholin-4- ylpyrimidin-4-yl]amine |
| 96 | 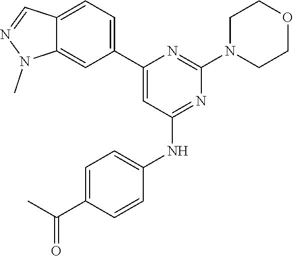 [SMILES: CC(=O)c1ccc(Nc2cc(-c3ccc4cnn(C)c4c3)nc(N3CCOCC3)n2)cc1] | 1-acetyl-4-{[6-(1-methyl(1H-indazol-6- yl))-2-morpholin-4-ylpyrimidin-4- yl]amino}benzene |
| 97 | 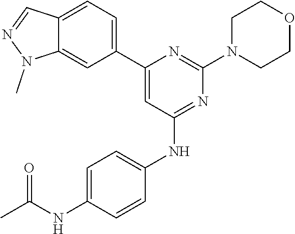 [SMILES: CC(=O)Nc1ccc(Nc2cc(-c3ccc4cnn(C)c4c3)nc(N3CCOCC3)n2)cc1] | N-(4-{[6-(1-methyl(1H-indazol-6-yl))- 2-morpholin-4-ylpyrimidin-4- yl]amino}phenyl)acetamide |
| 98 | 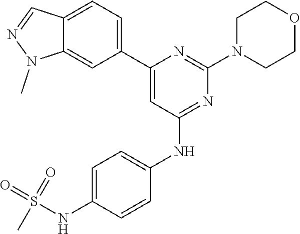 [SMILES: Cn1ncc2ccc(-c3cc(Nc4ccc(NS(C)(=O)=O)cc4)nc(N4CCOCC4)n3)cc21] | (4-{[6-(1-methyl(1H-indazol-6-yl))-2- morpholin-4-ylpyrimidin-4- yl]amino}phenyl)(methylsulfonyl)amine |
| 99 | 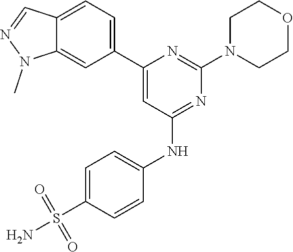 [SMILES: Cn1ncc2ccc(-c3cc(Nc4ccc(S(N)(=O)=O)cc4)nc(N4CCOCC4)n3)cc21] | 4-{[6-(1-methyl(1H-indazol-6-yl))-2- morpholin-4-ylpyrimidin-4- yl]amino}benzenesulfonamide |
| 100 | 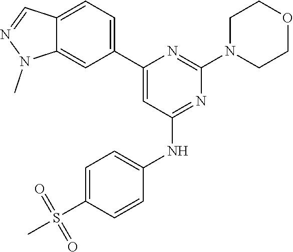 [SMILES: Cn1ncc2ccc(-c3cc(Nc4ccc(S(C)(=O)=O)cc4)nc(N4CCOCC4)n3)cc21] | 1-{[6-(1-methyl(1H-indazol-6-yl))-2- morpholin-4-ylpyrimidin-4-yl]amino}- 4-(methylsulfonyl)benzene |
| 101 | 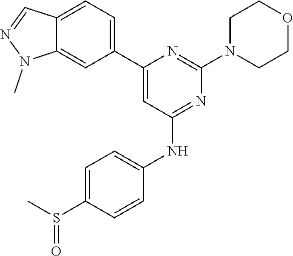 [SMILES: Cn1ncc2ccc(-c3cc(Nc4ccc(S(C)=O)cc4)nc(N4CCOCC4)n3)cc21] | 1-{[6-(1-methyl(1H-indazol-6-yl))-2- morpholin-4-ylpyrimidin-4-yl]amino}- 4-(methylsulfinyl)benzene |
| 102 | 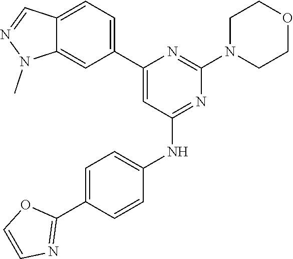 [SMILES: Cn1ncc2ccc(-c3cc(Nc4ccc(-c5ncco5)cc4)nc(N4CCOCC4)n3)cc21] | [6-(1-methyl(1H-indazol-6-yl))-2- morpholin-4-ylpyrimidin-4-yl](4-(1,3- oxazol-2-yl)phenyl)amine |
| 103 | 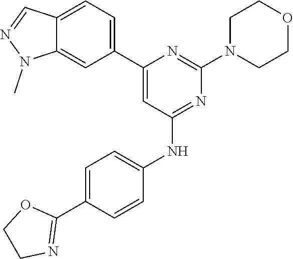 [SMILES: Cn1ncc2ccc(-c3cc(Nc4ccc(C5=NCCO5)cc4)nc(N4CCOCC4)n3)cc21] | [6-(1-methyl(1H-indazol-6-yl))-2- morpholin-4-ylpyrimidin-4-yl](4-(1,3- oxazolin-2-yl)phenyl)amine |
| 104 | 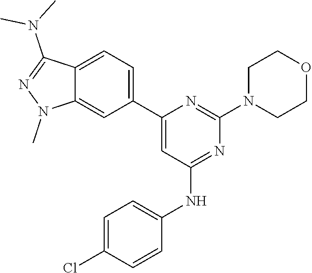 [SMILES: CN(C)c1nn(C)c2cc(-c3cc(Nc4ccc(Cl)cc4)nc(N4CCOCC4)n3)ccc12] | (6-{6-[(4-chlorophenyl)amino]-2- morpholin-4-ylpyrimidin-4-yl}-1- methyl(1H-indazol-3-yl))dimethylamine |
| 105 | 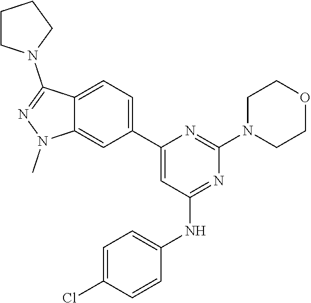 [SMILES: Cn1nc(N2CCCC2)c2ccc(-c3cc(Nc4ccc(Cl)cc4)nc(N4CCOCC4)n3)cc21] | (4-chlorophenyl)[6-(1-methyl-3- pyrrolidinyl(1H-indazol-6-yl))-2- morpholin-4-ylpyrimidin-4-yl]amine |
| 106 | 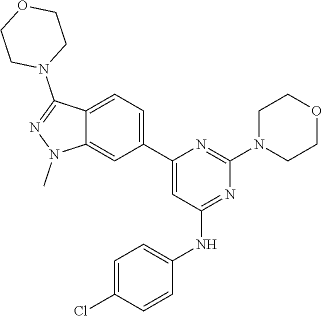 [SMILES: Cn1nc(N2CCOCC2)c2ccc(-c3cc(Nc4ccc(Cl)cc4)nc(N4CCOCC4)n3)cc21] | (4-chlorophenyl)[6-(1-methyl-3- morpholin-4-yl(1H-indazol-6-yl))-2- morpholin-4-ylpyrimidin-4-yl]amine |
| 107 | 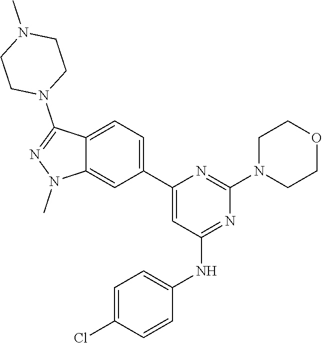 [SMILES: CN1CCN(c2nn(C)c3cc(-c4cc(Nc5ccc(Cl)cc5)nc(N5CCOCC5)n4)ccc23)CC1] | (4-chlorophenyl){6-[1-methyl-3-(4- methylpiperazinyl)(1H-indazol-6-yl)]-2- morpholin-4-ylpyrimidin-4-yl}amine |
| 108 | 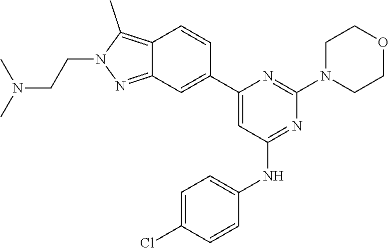 [SMILES: Cc1c2ccc(-c3cc(Nc4ccc(Cl)cc4)nc(N4CCOCC4)n3)cc2nn1CCN(C)C] | [2-(6-{6-[(4-chlorophenyl)amino]-2- morpholin-4-ylpyrimidin-4-yl}-3- methyl(2H-indazol-2- yl))ethyl]dimethylamine |
| 109 | 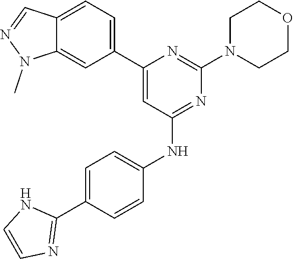 [SMILES: Cn1ncc2ccc(-c3cc(Nc4ccc(-c5ncc[nH]5)cc4)nc(N4CCOCC4)n3)cc21] | (4-imidazol-2-ylphenyl)[6-(1- methyl(1H-indazol-6-yl))-2-morpholin- 4-ylpyrimidin-4-yl]amine |
| 110 | 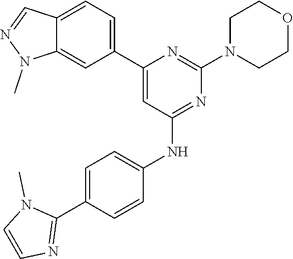 [SMILES: Cn1ccnc1-c1ccc(Nc2cc(-c3ccc4cnn(C)c4c3)nc(N3CCOCC3)n2)cc1] | [6-(1-methyl(1H-indazol-6-yl))-2- morpholin-4-ylpyrimidin-4-yl][4-(1- methylimidazol-2-yl)phenyl]amine |
| 111 | 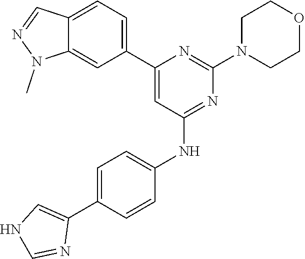 [SMILES: Cn1ncc2ccc(-c3cc(Nc4ccc(-c5c[nH]cn5)cc4)nc(N4CCOCC4)n3)cc21] | (4-imidazol-4-ylphenyl)[6-(1- methyl(1H-indazol-6-yl))-2-morpholin- 4-ylpyrimidin-4-yl]amine |
| 112 | 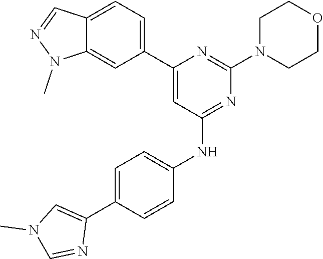 [SMILES: Cn1cnc(-c2ccc(Nc3cc(-c4ccc5cnn(C)c5c4)nc(N4CCOCC4)n3)cc2)c1] | [6-(1-methyl(1H-indazol-6-yl))-2- morpholin-4-ylpyrimidin-4-yl][4-(1- methylimidazol-4-yl)phenyl]amine |
| 113 | 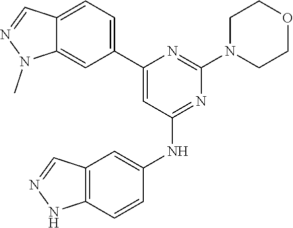 [SMILES: Cn1ncc2ccc(-c3cc(Nc4ccc5[nH]ncc5c4)nc(N4CCOCC4)n3)cc21] | 1H-indazol-5-yl[6-(1-methyl(1H- indazol-6-yl))-2-morpholin-4- ylpyrimidin-4-yl]amine |
| 114 | 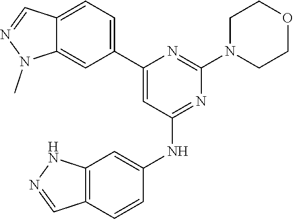 [SMILES: Cn1ncc2ccc(-c3cc(Nc4ccc5cn[nH]c5c4)nc(N4CCOCC4)n3)cc21] | 1H-indazol-6-yl[6-(1-methyl(1H- indazol-6-yl))-2-morpholin-4- ylpyrimidin-4-yl]amine |
| 115 | 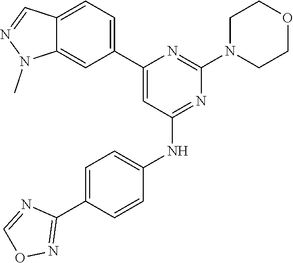 [SMILES: Cn1ncc2ccc(-c3cc(Nc4ccc(-c5ncon5)cc4)nc(N4CCOCC4)n3)cc21] | [6-(1-methyl(1H-indazol-6-yl))-2- morpholin-4-ylpyrimidin-4-yl](4-(1,2,4- oxadiazol-3-yl)phenyl)amine |
| 116 | 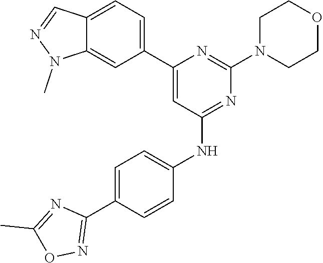 [SMILES: Cc1nc(-c2ccc(Nc3cc(-c4ccc5cnn(C)c5c4)nc(N4CCOCC4)n3)cc2)no1] | [4-(5-methyl(1,2,4-oxadiazol-3- yl))phenyl][6-(1-methyl(1H-indazol-6- yl))-2-morpholin-4-ylpyrimidin-4- yl]amine |
| 117 | 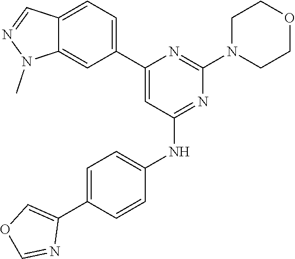 [SMILES: Cn1ncc2ccc(-c3cc(Nc4ccc(-c5cocn5)cc4)nc(N4CCOCC4)n3)cc21] | [6-(1-methyl(1H-indazol-6-yl))-2- morpholin-4-ylpyrimidin-4-yl](4-(1,3- oxazol-4-yl)phenyl)amine |
| 118 | 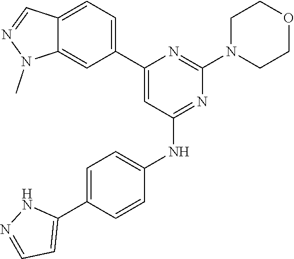 [SMILES: Cn1ncc2ccc(-c3cc(Nc4ccc(-c5ccn[nH]5)cc4)nc(N4CCOCC4)n3)cc21] | [6-(1-methyl(1H-indazol-6-yl))-2- morpholin-4-ylpyrimidin-4-yl](4- pyrazol-5-ylphenyl)amine |
| 119 | 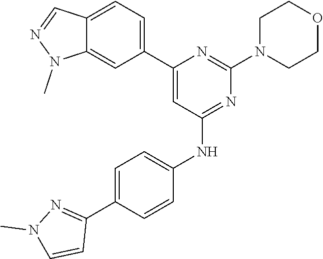 [SMILES: Cn1ccc(-c2ccc(Nc3cc(-c4ccc5cnn(C)c5c4)nc(N4CCOCC4)n3)cc2)n1] | [6-(1-methyl(1H-indazol-6-yl))-2- morpholin-4-ylpyrimidin-4-yl][4-(1- methylpyrazol-3-yl)phenyl]amine |
| 120 | 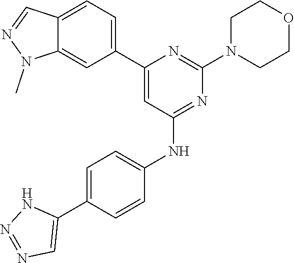 [SMILES: Cn1ncc2ccc(-c3cc(Nc4ccc(-c5cnn[nH]5)cc4)nc(N4CCOCC4)n3)cc21] | (4-(1H-1,2,3-triazol-5-yl)phenyl)[6-(1- methyl(1H-indazol-6-yl))-2-morpholin- 4-ylpyrimidin-4-yl]amine |
| 121 | 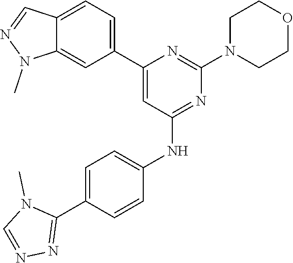 [SMILES: Cn1cnnc1-c1ccc(Nc2cc(-c3ccc4cnn(C)c4c3)nc(N3CCOCC3)n2)cc1] | [4-(4-methyl(1,2,4-triazol-3- yl))phenyl][6-(1-methyl(1H-indazol-6- yl))-2-morpholin-4-ylpyrimidin-4- yl]amine |
| 122 | 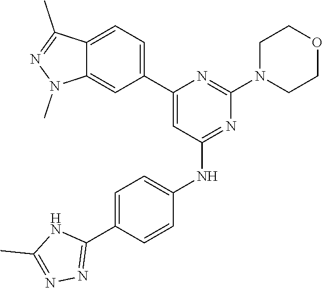 [SMILES: Cc1nnc(-c2ccc(Nc3cc(-c4ccc5c(C)nn(C)c5c4)nc(N4CCOCC4)n3)cc2)[nH]1] | [6-(1,3-dimethyl(1H-indazol-6-yl))-2- morpholin-4-ylpyrimidin-4-yl][4-(5- methyl(4H-1,2,4-triazol-3- yl))phenyl]amine |
| 123 | 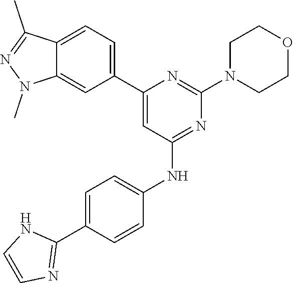 [SMILES: Cc1nn(C)c2cc(-c3cc(Nc4ccc(-c5ncc[nH]5)cc4)nc(N4CCOCC4)n3)ccc12] | [6-(1,3-dimethyl(1H-indazol-6-yl))-2- morpholin-4-ylpyrimidin-4-yl](4- imidazol-2-ylphenyl)amine |
| 124 | 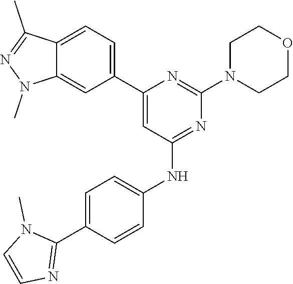 [SMILES: Cc1nn(C)c2cc(-c3cc(Nc4ccc(-c5nccn5C)cc4)nc(N4CCOCC4)n3)ccc12] | [6-(1,3-dimethyl(1H-indazol-6-yl))-2- morpholin-4-ylpyrimidin-4-yl][4-(1- methylimidazol-2-yl)phenyl]amine |
| 125 | 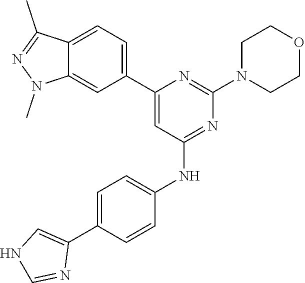 [SMILES: Cc1nn(C)c2cc(-c3cc(Nc4ccc(-c5c[nH]cn5)cc4)nc(N4CCOCC4)n3)ccc12] | [6-(1,3-dimethyl(1H-indazol-6-yl))-2- morpholin-4-ylpyrimidin-4-yl](4- imidazol-4-ylphenyl)amine |
| 126 | 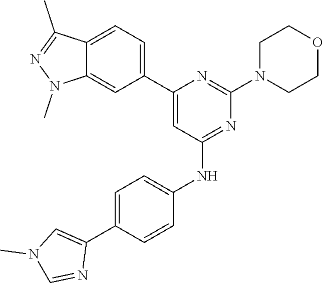 [SMILES: Cc1nn(C)c2cc(-c3cc(Nc4ccc(-c5cn(C)cn5)cc4)nc(N4CCOCC4)n3)ccc12] | [6-(1,3-dimethyl(1H-indazol-6-yl))-2- morpholin-4-ylpyrimidin-4-yl][4-(1- methylimidazol-4-yl)phenyl]amine |
| 127 | 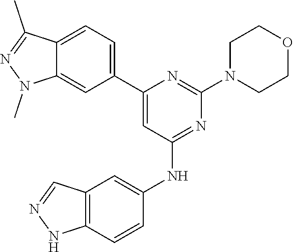 [SMILES: Cc1nn(C)c2cc(-c3cc(Nc4ccc5[nH]ncc5c4)nc(N4CCOCC4)n3)ccc12] | 1H-indazol-5-yl[6-(1,3-dimethyl(1H- indazol-6-yl))-2-morpholin-4- ylpyrimidin-4-yl]amine |
| 128 | 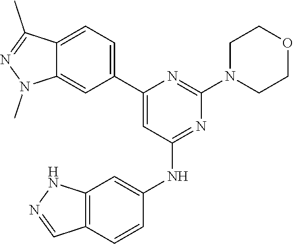 [SMILES: Cc1nn(C)c2cc(-c3cc(Nc4ccc5cn[nH]c5c4)nc(N4CCOCC4)n3)ccc12] | 1H-indazol-6-yl[6-(1,3-dimethyl(1H- indazol-6-yl))-2-morpholin-4- ylpyrimidin-4-yl]amine |
| 129 | 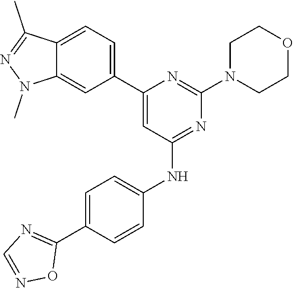 [SMILES: Cc1nn(C)c2cc(-c3cc(Nc4ccc(-c5ncno5)cc4)nc(N4CCOCC4)n3)ccc12] | [6-(1,3-dimethyl(1H-indazol-6-yl))-2- morpholin-4-ylpyrimidin-4-yl](4-(1,2,4- oxadiazol-5-yl)phenyl)amine |
| 130 | 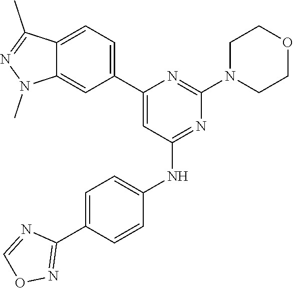 [SMILES: Cc1nn(C)c2cc(-c3cc(Nc4ccc(-c5ncon5)cc4)nc(N4CCOCC4)n3)ccc12] | [6-(1,3-dimethyl(1H-indazol-6-yl))-2- morpholin-4-ylpyrimidin-4-yl](4-(1,2,4- oxadiazol-3-yl)phenyl)amine |
| 131 | 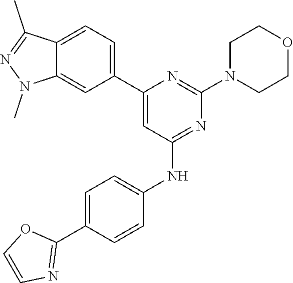 [SMILES: Cc1nn(C)c2cc(-c3cc(Nc4ccc(-c5ncco5)cc4)nc(N4CCOCC4)n3)ccc12] | [6-(1,3-dimethyl(1H-indazol-6-yl))-2- morpholin-4-ylpyrimidin-4-yl](4-(1,3- oxazol-2-yl)phenyl)amine |
| 132 | 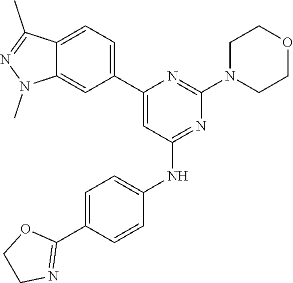 [SMILES: Cc1nn(C)c2cc(-c3cc(Nc4ccc(C5=NCCO5)cc4)nc(N4CCOCC4)n3)ccc12] | [6-(1,3-dimethyl(1H-indazol-6-yl))-2- morpholin-4-ylpyrimidin-4-yl](4-(1,3- oxazolin-2-yl)phenyl)amine |
| 133 | 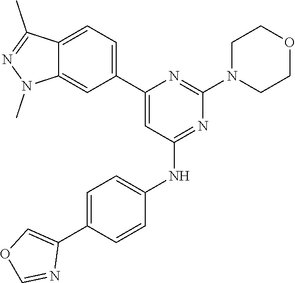 [SMILES: Cc1nn(C)c2cc(-c3cc(Nc4ccc(-c5cocn5)cc4)nc(N4CCOCC4)n3)ccc12] | [6-(1,3-dimethyl(1H-indazol-6-yl))-2- morpholin-4-ylpyrimidin-4-yl](4-(1,3- oxazol-4-yl)phenyl)amine |
| 134 | 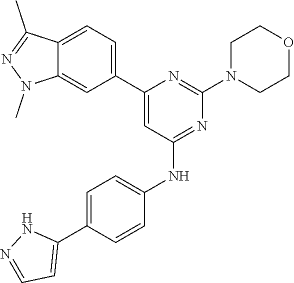 [SMILES: Cc1nn(C)c2cc(-c3cc(Nc4ccc(-c5ccn[nH]5)cc4)nc(N4CCOCC4)n3)ccc12] | [6-(1,3-dimethyl(1H-indazol-6-yl))-2- morpholin-4-ylpyrimidin-4-yl](4- pyrazol-5-ylphenyl)amine |
| 135 | 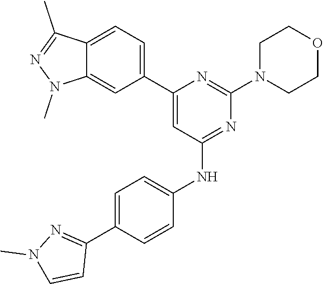 [SMILES: Cc1nn(C)c2cc(-c3cc(Nc4ccc(-c5ccn(C)n5)cc4)nc(N4CCOCC4)n3)ccc12] | [6-(1,3-dimethyl(1H-indazol-6-yl))-2- morpholin-4-ylpyrimidin-4-yl][4-(1- methylpyrazol-3-yl)phenyl]amine |
| 136 | 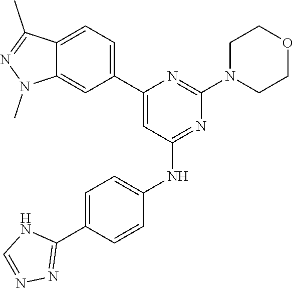 [SMILES: Cc1nn(C)c2cc(-c3cc(Nc4ccc(-c5nnc[nH]5)cc4)nc(N4CCOCC4)n3)ccc12] | (4-(4H-1,2,4-triazol-3-yl)phenyl)[6- (1,3-dimethyl(1H-indazol-6-yl))-2- morpholin-4-ylpyrimidin-4-yl]amine |
| 137 | 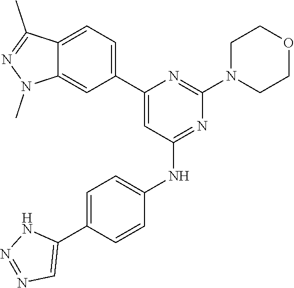 [SMILES: Cc1nn(C)c2cc(-c3cc(Nc4ccc(-c5cnn[nH]5)cc4)nc(N4CCOCC4)n3)ccc12] | (4-(1H-1,2,3-triazol-5-yl)phenyl)[6- (1,3-dimethyl(1H-indazol-6-yl))-2- morpholin-4-ylpyrimidin-4-yl]amine |
| 138 | 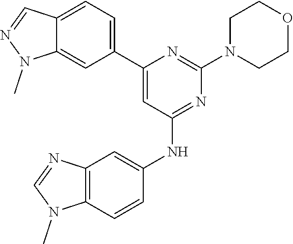 [SMILES: Cn1cnc2cc(Nc3cc(-c4ccc5cnn(C)c5c4)nc(N4CCOCC4)n3)ccc21] | [6-(1-methyl(1H-indazol-6-yl))-2- morpholin-4-ylpyrimidin-4-yl](1- methylbenzimidazol-5-yl)amine |
| 139 | 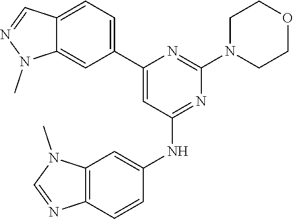 [SMILES: Cn1cnc2ccc(Nc3cc(-c4ccc5cnn(C)c5c4)nc(N4CCOCC4)n3)cc21] | [6-(1-methyl(1H-indazol-6-yl))-2- morpholin-4-ylpyrimidin-4-yl](1- methylbenzimidazol-6-yl)amine |
| 140 | 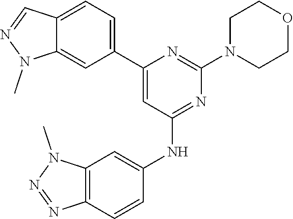 [SMILES: Cn1ncc2ccc(-c3cc(Nc4ccc5nnn(C)c5c4)nc(N4CCOCC4)n3)cc21] | [6-(1-methyl(1H-indazol-6-yl))-2- morpholin-4-ylpyrimidin-4-yl](1- methylbenzotriazol-6-yl)amine |
| 141 | 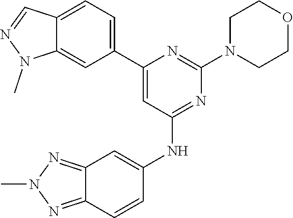 [SMILES: Cn1nc2ccc(Nc3cc(-c4ccc5cnn(C)c5c4)nc(N4CCOCC4)n3)cc2n1] | [6-(1-methyl(1H-indazol-6-yl))-2- morpholin-4-ylpyrimidin-4-yl](2- methyl(2-hydrobenzotriazol-5-yl))amine |
| 142 | 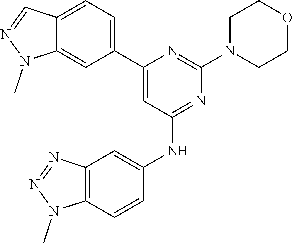 [SMILES: Cn1ncc2ccc(-c3cc(Nc4ccc5c(c4)nnn5C)nc(N4CCOCC4)n3)cc21] | [6-(1-methyl(1H-indazol-6-yl))-2- morpholin-4-ylpyrimidin-4-yl](1- methylbenzotriazol-5-yl)amine |
| 143 | 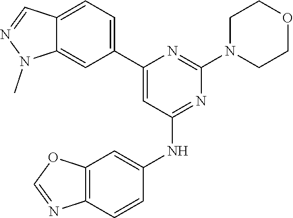 [SMILES: Cn1ncc2ccc(-c3cc(Nc4ccc5ncoc5c4)nc(N4CCOCC4)n3)cc21] | benzoxazol-6-yl[6-(1-methyl(1H- indazol-6-yl))-2-morpholin-4- ylpyrimidin-4-yl]amine |
| 144 | 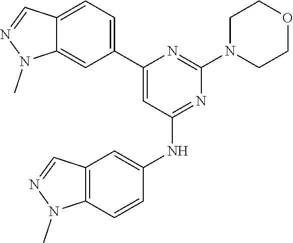 [SMILES: Cn1ncc2cc(Nc3cc(-c4ccc5cnn(C)c5c4)nc(N4CCOCC4)n3)ccc21] | (1-methyl(1H-indazol-5-yl))[6-(1- methyl(1H-indazol-6-yl))-2-morpholin- 4-ylpyrimidin-4-yl]amine |
| 145 | 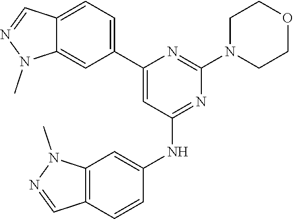 [SMILES: Cn1ncc2ccc(Nc3cc(-c4ccc5cnn(C)c5c4)nc(N4CCOCC4)n3)cc21] | (1-methyl(1H-indazol-6-yl))[6-(1- methyl(1H-indazol-6-yl))-2-morpholin- 4-ylpyrimidin-4-yl]amine |
| 146 | 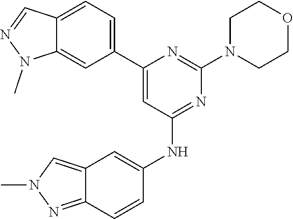 [SMILES: Cn1cc2cc(Nc3cc(-c4ccc5cnn(C)c5c4)nc(N4CCOCC4)n3)ccc2n1] | [6-(1-methyl(1H-indazol-6-yl))-2- morpholin-4-ylpyrimidin-4-yl](2- methyl(2H-indazol-5-yl))amine |
| 147 | 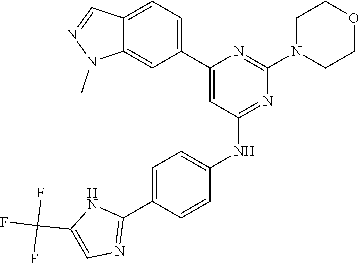 [SMILES: Cn1ncc2ccc(-c3cc(Nc4ccc(-c5ncc(C(F)(F)F)[nH]5)cc4)nc(N4CCOCC4)n3)cc21] | [6-(1-methyl(1H-indazol-6-yl))-2- morpholin-4-ylpyrimidin-4-yl]{4-[5- (trifluoromethyl)imidazol-2- yl]phenyl}amine |
| 148 | 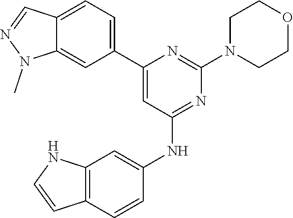 [SMILES: Cn1ncc2ccc(-c3cc(Nc4ccc5cc[nH]c5c4)nc(N4CCOCC4)n3)cc21] | indol-6-yl[6-(1-methyl(1H-indazol-6- yl))-2-morpholin-4-ylpyrimidin-4- yl]amine |
| 149 | 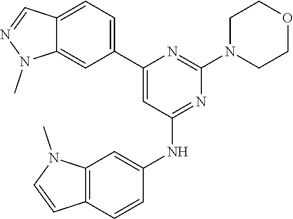 [SMILES: Cn1ccc2ccc(Nc3cc(-c4ccc5cnn(C)c5c4)nc(N4CCOCC4)n3)cc21] | [6-(1-methyl(1H-indazol-6-yl))-2- morpholin-4-ylpyrimidin-4-yl](1- methylindol-6-yl)amine |
| 150 | 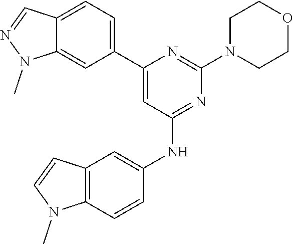 [SMILES: Cn1ccc2cc(Nc3cc(-c4ccc5cnn(C)c5c4)nc(N4CCOCC4)n3)ccc21] | [6-(1-methyl(1H-indazol-6-yl))-2- morpholin-4-ylpyrimidin-4-yl](1- methylindol-5-yl)amine |
| 151 | 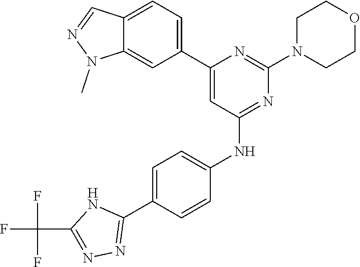 [SMILES: Cn1ncc2ccc(-c3cc(Nc4ccc(-c5nnc(C(F)(F)F)[nH]5)cc4)nc(N4CCOCC4)n3)cc21] | [6-(1-methyl(1H-indazol-6-yl))-2- morpholin-4-ylpyrimidin-4-yl]{4-[5- (trifluoromethyl)(4H-1,2,4-triazol-3- yl)]phenyl}amine |
| 152 | 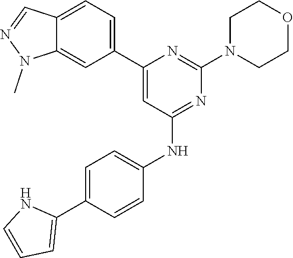 [SMILES: Cn1ncc2ccc(-c3cc(Nc4ccc(-c5ccc[nH]5)cc4)nc(N4CCOCC4)n3)cc21] | [6-(1-methyl(1H-indazol-6-yl))-2- morpholin-4-ylpyrimidin-4-yl](4-pyrrol- 2-ylphenyl)amine |
| 153 | 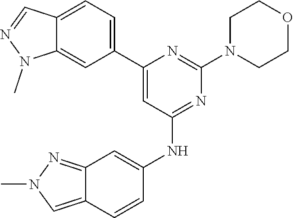 [SMILES: Cn1cc2ccc(Nc3cc(-c4ccc5cnn(C)c5c4)nc(N4CCOCC4)n3)cc2n1] | [6-(1-methyl(1H-indazol-6-yl))-2- morpholin-4-ylpyrimidin-4-yl](2- methyl(2H-indazol-6-yl))amine |
| 154 | 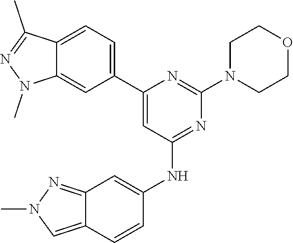 [SMILES: Cc1nn(C)c2cc(-c3cc(Nc4ccc5cn(C)nc5c4)nc(N4CCOCC4)n3)ccc12] | [6-(1,3-dimethyl(1H-indazol-6-yl))-2- morpholin-4-ylpyrimidin-4-yl](2- methyl(2H-indazol-6-yl))amine |
| 155 | 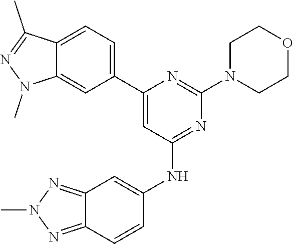 [SMILES: Cc1nn(C)c2cc(-c3cc(Nc4ccc5nn(C)nc5c4)nc(N4CCOCC4)n3)ccc12] | [6-(1,3-dimethyl(1H-indazol-6-yl))-2- morpholin-4-ylpyrimidin-4-yl](2- methyl(2-hydrobenzotriazol-5-yl))amine |
| 156 | 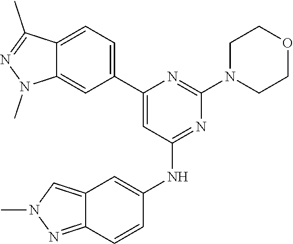 [SMILES: Cc1nn(C)c2cc(-c3cc(Nc4ccc5nn(C)cc5c4)nc(N4CCOCC4)n3)ccc12] | [6-(1,3-dimethyl(1H-indazol-6-yl))-2- morpholin-4-ylpyrimidin-4-yl](2- methyl(2H-indazol-5-yl))amine |
| 157 | 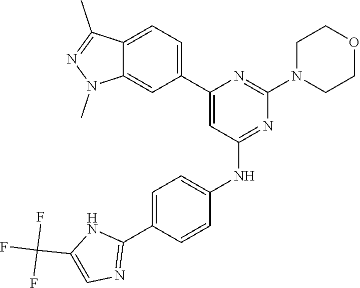 [SMILES: Cc1nn(C)c2cc(-c3cc(Nc4ccc(-c5ncc(C(F)(F)F)[nH]5)cc4)nc(N4CCOCC4)n3)ccc12] | [6-(1,3-dimethyl(1H-indazol-6-yl))-2- morpholin-4-ylpyrimidin-4-yl]{4-[5- (trifluoromethyl)imidazol-2- yl]phenyl}amine |
| 158 | 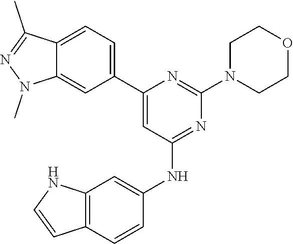 [SMILES: Cc1nn(C)c2cc(-c3cc(Nc4ccc5cc[nH]c5c4)nc(N4CCOCC4)n3)ccc12] | [6-(1,3-dimethyl(1H-indazol-6-yl))-2- morpholin-4-ylpyrimidin-4-yl]indol-6- ylamine |
| 159 | 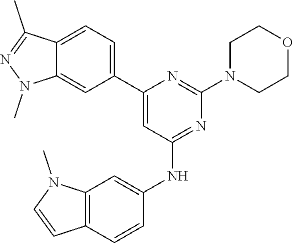 [SMILES: Cc1nn(C)c2cc(-c3cc(Nc4ccc5ccn(C)c5c4)nc(N4CCOCC4)n3)ccc12] | [6-(1,3-dimethyl(1H-indazol-6-yl))-2- morpholin-4-ylpyrimidin-4-yl](1- methylindol-6-yl)amine |
| 160 | 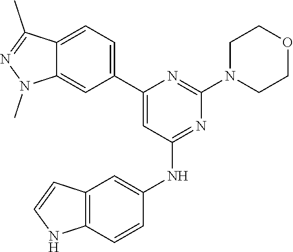 [SMILES: Cc1nn(C)c2cc(-c3cc(Nc4ccc5[nH]ccc5c4)nc(N4CCOCC4)n3)ccc12] | [6-(1,3-dimethyl(1H-indazol-6-yl))-2- morpholin-4-ylpyrimidin-4-yl]indol-5- ylamine |
| 161 | 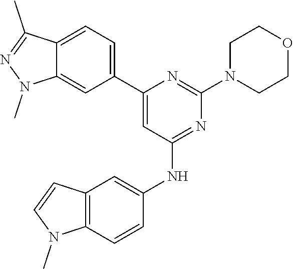 [SMILES: Cc1nn(C)c2cc(-c3cc(Nc4ccc5c(ccn5C)c4)nc(N4CCOCC4)n3)ccc12] | [6-(1,3-dimethyl(1H-indazol-6-yl))-2- morpholin-4-ylpyrimidin-4-yl](1- methylindol-5-yl)amine |
| 162 | 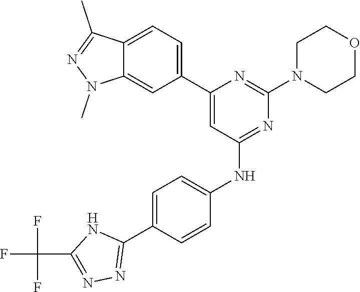 [SMILES: Cc1nn(C)c2cc(-c3cc(Nc4ccc(-c5nnc(C(F)(F)F)[nH]5)cc4)nc(N4CCOCC4)n3)ccc12] | [6-(1,3-dimethyl(1H-indazol-6-yl))-2- morpholin-4-ylpyrimidin-4-yl]{4-[5- (trifluoromethyl)(4H-1,2,4-triazol-3- yl)]phenyl}amine |
| 163 | 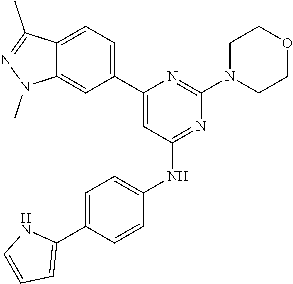 [SMILES: Cc1nn(C)c2cc(-c3cc(Nc4ccc(-c5ccc[nH]5)cc4)nc(N4CCOCC4)n3)ccc12] | [6-(1,3-dimethyl(1H-indazol-6-yl))-2- morpholin-4-ylpyrimidin-4-yl](4-pyrrol- 2-ylphenyl)amine |
| 164 | 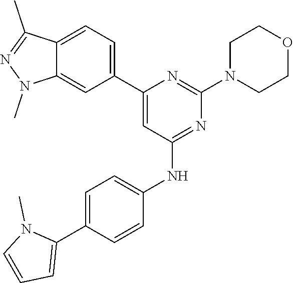 [SMILES: Cc1nn(C)c2cc(-c3cc(Nc4ccc(-c5cccn5C)cc4)nc(N4CCOCC4)n3)ccc12] | [6-(1,3-dimethyl(1H-indazol-6-yl))-2- morpholin-4-ylpyrimidin-4-yl][4-(1- methylpyrrol-2-yl)phenyl]amine |
| 165 | 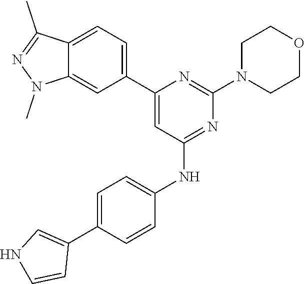 [SMILES: Cc1nn(C)c2cc(-c3cc(Nc4ccc(-c5cc[nH]c5)cc4)nc(N4CCOCC4)n3)ccc12] | [6-(1,3-dimethyl(1H-indazol-6-yl))-2- morpholin-4-ylpyrimidin-4-yl](4-pyrrol- 3-ylphenyl)amine |
| 166 | 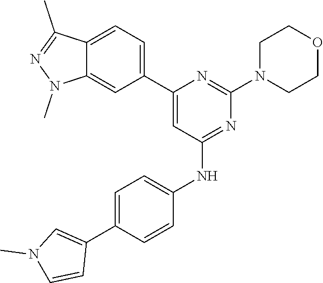 [SMILES: Cc1nn(C)c2cc(-c3cc(Nc4ccc(-c5ccn(C)c5)cc4)nc(N4CCOCC4)n3)ccc12] | [6-(1,3-dimethyl(1H-indazol-6-yl))-2- morpholin-4-ylpyrimidin-4-yl][4-(1- methylpyrrol-3-yl)phenyl]amine |
| 167 | 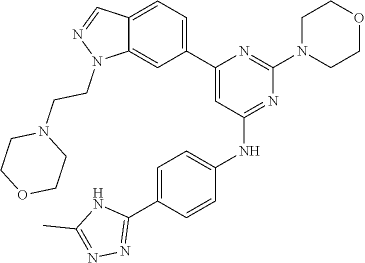 [SMILES: Cc1nnc(-c2ccc(Nc3cc(-c4ccc5cnn(CCN6CCOCC6)c5c4)nc(N4CCOCC4)n3)cc2)[nH]1] | [4-(5-methyl(4H-1,2,4-triazol-3- yl))phenyl]{2-morpholin-4-yl-6-[1-(2- morpholin-4-ylethyl)(1H-indazol-6- yl)]pyrimidin-4-yl}amine |
| 168 | 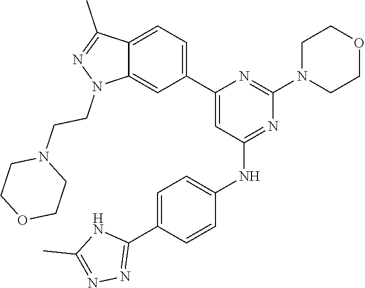 [SMILES: Cc1nnc(-c2ccc(Nc3cc(-c4ccc5c(C)nn(CCN6CCOCC6)c5c4)nc(N4CCOCC4)n3)cc2)[nH]1] | [4-(5-methyl(4H-1,2,4-triazol-3- yl))phenyl]{6-[3-methyl-1-(2- morpholin-4-ylethyl)(1H-indazol-6-yl)]- 2-morpholin-4-ylpyrimidin-4-yl}amine |
| 169 | 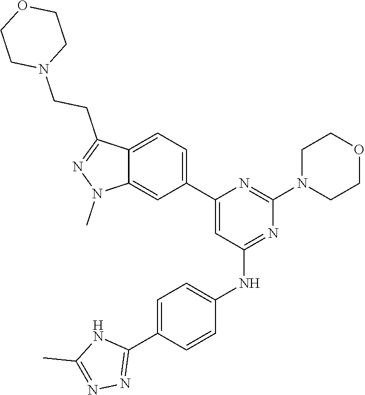 [SMILES: Cc1nnc(-c2ccc(Nc3cc(-c4ccc5c(CCN6CCOCC6)nn(C)c5c4)nc(N4CCOCC4)n3)cc2)[nH]1] | [4-(5-methyl(4H-1,2,4-triazol-3- yl))phenyl]{6-[1-methyl-3-(2- morpholin-4-ylethyl)(1H-indazol-6-yl)]- 2-morpholin-4-ylpyrimidin-4-yl}amine |
| 170 | 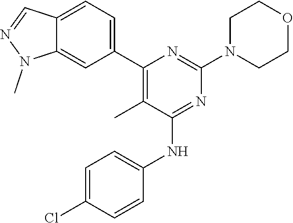 [SMILES: Cc1c(Nc2ccc(Cl)cc2)nc(N2CCOCC2)nc1-c1ccc2cnn(C)c2c1] | (4-chlorophenyl)[5-methyl-6-(1- methyl(1H-indazol-6-yl))-2-morpholin- 4-ylpyrimidin-4-yl]amine |
| 171 | 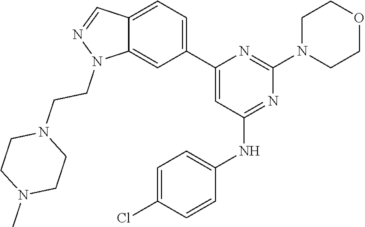 [SMILES: CN1CCN(CCn2ncc3ccc(-c4cc(Nc5ccc(Cl)cc5)nc(N5CCOCC5)n4)cc32)CC1] | (4-chlorophenyl)(6-{1-[2-(4- methylpiperazinyl)ethyl](1H-indazol-6- yl)}-2-morpholin-4-ylpyrimidin-4- yl)amine |
| 172 | 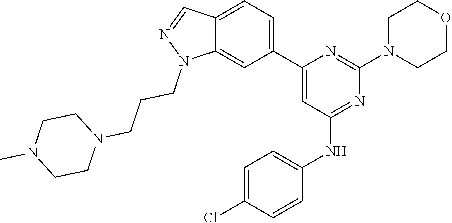 [SMILES: CN1CCN(CCCn2ncc3ccc(-c4cc(Nc5ccc(Cl)cc5)nc(N5CCOCC5)n4)cc32)CC1] | (4-chlorophenyl)(6-{1-[3-(4- methylpiperazinyl)propyl](1H-indazol- 6-yl)}-2-morpholin-4-ylpyrimidin-4- yl)amine |
| 173 | 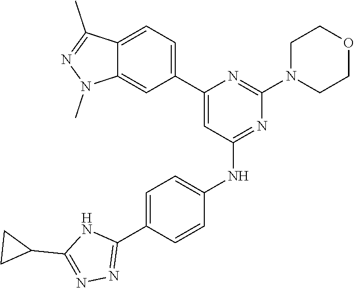 [SMILES: Cc1nn(C)c2cc(-c3cc(Nc4ccc(-c5nnc(C6CC6)[nH]5)cc4)nc(N4CCOCC4)n3)ccc12] | [6-(1,3-dimethyl(1H-indazol-6-yl))-2- morpholin-4-ylpyrimidin-4-yl][4-(5- cyclopropyl(4H-1,2,4-triazol-3- yl))phenyl]amine |
| 174 | 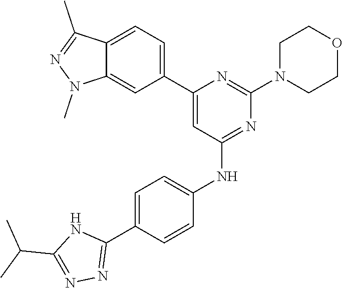 [SMILES: Cc1nn(C)c2cc(-c3cc(Nc4ccc(-c5nnc(C(C)C)[nH]5)cc4)nc(N4CCOCC4)n3)ccc12] | [6-(1,3-dimethyl(1H-indazol-6-yl))-2- morpholin-4-ylpyrimidin-4-yl]{4-[5- (methylethyl)(4H-1,2,4-triazol-3- yl)]phenyl}amine |
| 175 | 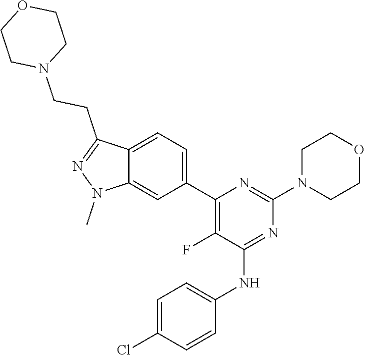 [SMILES: Cn1nc(CCN2CCOCC2)c2ccc(-c3nc(N4CCOCC4)nc(Nc4ccc(Cl)cc4)c3F)cc21] | (4-chlorophenyl){5-fluoro-6-[1-methyl- 3-(2-morpholin-4-ylethyl)(1H-indazol- 6-yl)]-2-morpholin-4-ylpyrimidin-4- yl}amine |
| 176 | 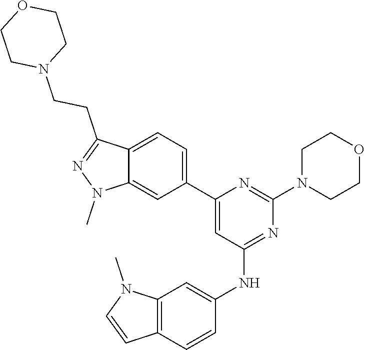 [SMILES: Cn1ccc2ccc(Nc3cc(-c4ccc5c(CCN6CCOCC6)nn(C)c5c4)nc(N4CCOCC4)n3)cc21] | (1-methylindol-6-yl){6-[1-methyl-3-(2- morpholin-4-ylethyl)(1H-indazol-6-yl)]- 2-morpholin-4-ylpyrimidin-4-yl}amine |
| 177 | 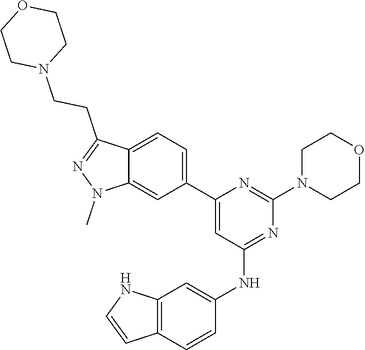 [SMILES: Cn1nc(CCN2CCOCC2)c2ccc(-c3cc(Nc4ccc5cc[nH]c5c4)nc(N4CCOCC4)n3)cc21] | indol-6-yl{6-[1-methyl-3-(2-morpholin- 4-ylethyl)(1H-indazol-6-yl)]-2- morpholin-4-ylpyrimidin-4-yl}amine |
| 178 | 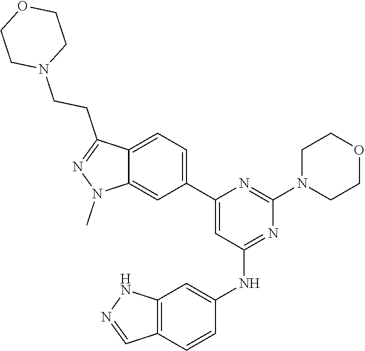 [SMILES: Cn1nc(CCN2CCOCC2)c2ccc(-c3cc(Nc4ccc5cn[nH]c5c4)nc(N4CCOCC4)n3)cc21] | 1H-indazol-6-yl{6-[1-methyl-3-(2- morpholin-4-ylethyl)(1H-indazol-6-yl)]- 2-morpholin-4-ylpyrimidin-4-yl}amine |
| 179 | 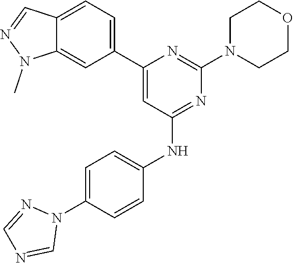 [SMILES: Cn1ncc2ccc(-c3cc(Nc4ccc(-n5cncn5)cc4)nc(N4CCOCC4)n3)cc21] | [6-(1-methyl(1H-indazol-6-yl))-2- morpholin-4-ylpyrimidin-4-yl](4-(1,2,4- triazolyl)phenyl)amine |
| 180 | 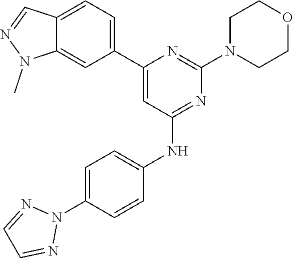 [SMILES: Cn1ncc2ccc(-c3cc(Nc4ccc(-n5nccn5)cc4)nc(N4CCOCC4)n3)cc21] | [6-(1-methyl(1H-indazol-6-yl))-2- morpholin-4-ylpyrimidin-4-yl](4-(1,2,3- triazol-2-yl)phenyl)amine |
| 181 | 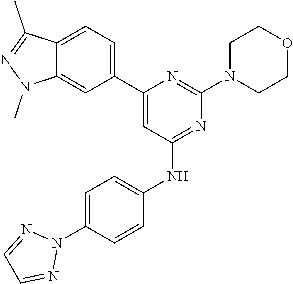 [SMILES: Cc1nn(C)c2cc(-c3cc(Nc4ccc(-n5nccn5)cc4)nc(N4CCOCC4)n3)ccc12] | [6-(1,3-dimethyl(1H-indazol-6-yl))-2- morpholin-4-ylpyrimidin-4-yl](4-(1,2,3- triazol-2-yl)phenyl)amine |
| 182 | 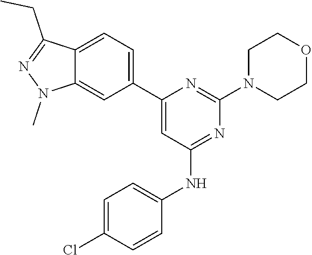 [SMILES: CCc1nn(C)c2cc(-c3cc(Nc4ccc(Cl)cc4)nc(N4CCOCC4)n3)ccc12] | (4-chlorophenyl)[6-(3-ethyl-1- methyl(1H-indazol-6-yl))-2-morpholin- 4-ylpyrimidin-4-yl]amine |
| 183 | 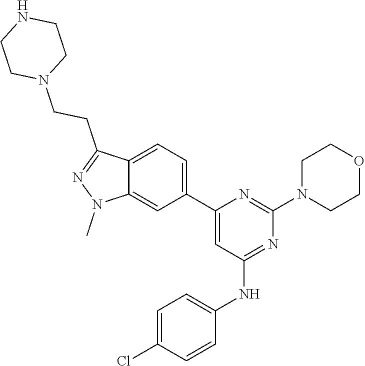 [SMILES: Cn1nc(CCN2CCNCC2)c2ccc(-c3cc(Nc4ccc(Cl)cc4)nc(N4CCOCC4)n3)cc21] | (4-chlorophenyl){6-[1-methyl-3-(2- piperazinylethyl)(1H-indazol-6-yl)]-2- morpholin-4-ylpyrimidin-4-yl}amine |
| 184 | 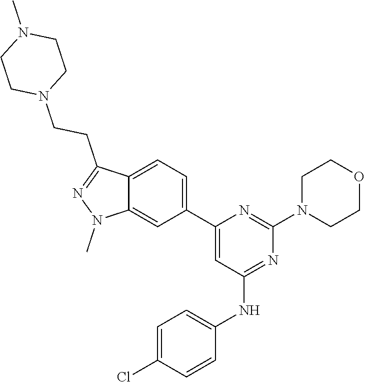 [SMILES: CN1CCN(CCc2nn(C)c3cc(-c4cc(Nc5ccc(Cl)cc5)nc(N5CCOCC5)n4)ccc23)CC1] | (4-chlorophenyl)(6-{1-methyl-3-[2-(4- methylpiperazinyl)ethyl](1H-indazol-6- yl)}-2-morpholin-4-ylpyrimidin-4- yl)amine |
| 185 | 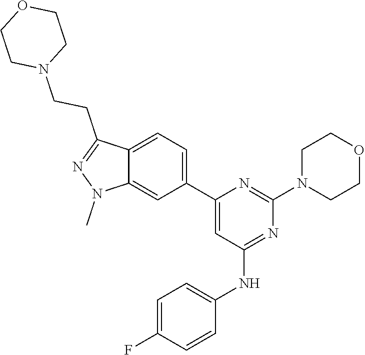 [SMILES: Cn1nc(CCN2CCOCC2)c2ccc(-c3cc(Nc4ccc(F)cc4)nc(N4CCOCC4)n3)cc21] | (4-fluorophenyl){6-[1-methyl-3-(2- morpholin-4-ylethyl)(1H-indazol-6-yl)]- 2-morpholin-4-ylpyrimidin-4-yl}amine |
| 186 | 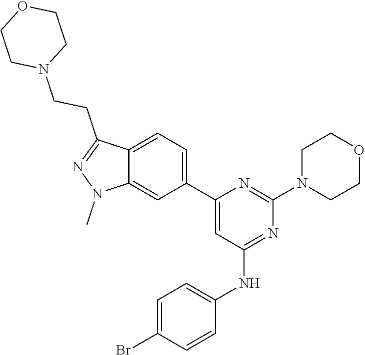 [SMILES: Cn1nc(CCN2CCOCC2)c2ccc(-c3cc(Nc4ccc(Br)cc4)nc(N4CCOCC4)n3)cc21] | (4-bromophenyl){6-[1-methyl-3-(2- morpholin-4-ylethyl)(1H-indazol-6-yl)]- 2-morpholin-4-ylpyrimidin-4-yl}amine |
| 187 | 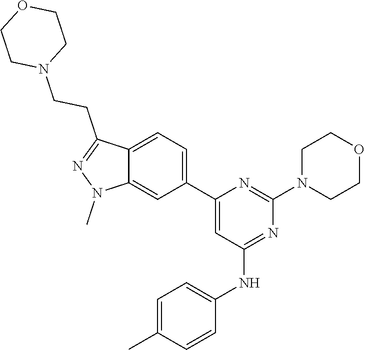 [SMILES: Cc1ccc(Nc2cc(-c3ccc4c(CCN5CCOCC5)nn(C)c4c3)nc(N3CCOCC3)n2)cc1] | {6-[1-methyl-3-(2-morpholin-4- ylethyl)(1H-indazol-6-yl)]-2-morpholin- 4-ylpyrimidin-4-yl}(4- methylphenyl)amine |
| 188 | 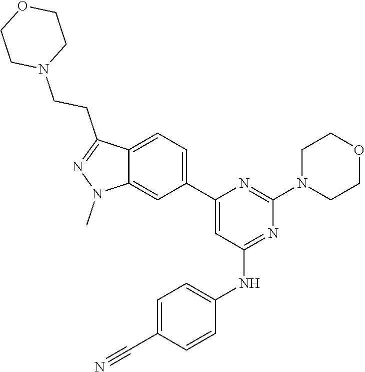 [SMILES: Cn1nc(CCN2CCOCC2)c2ccc(-c3cc(Nc4ccc(C#N)cc4)nc(N4CCOCC4)n3)cc21] | 4-({6-[1-methyl-3-(2-morpholin-4- ylethyl)(1H-indazol-6-yl)]-2-morpholin- 4-ylpyrimidin-4- yl}amino)benzenecarbonitrile |
| 189 | 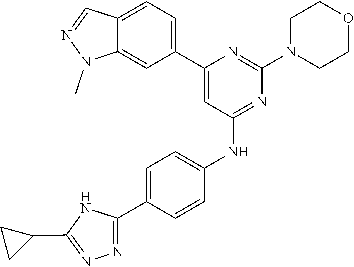 [SMILES: Cn1ncc2ccc(-c3cc(Nc4ccc(-c5nnc(C6CC6)[nH]5)cc4)nc(N4CCOCC4)n3)cc21] | [4-(5-cyclopropyl(4H-1,2,4-triazol-3- yl))phenyl][6-(1-methyl(1H-indazol-6- yl))-2-morpholin-4-ylpyrimidin-4- yl]amine |
| 190 | 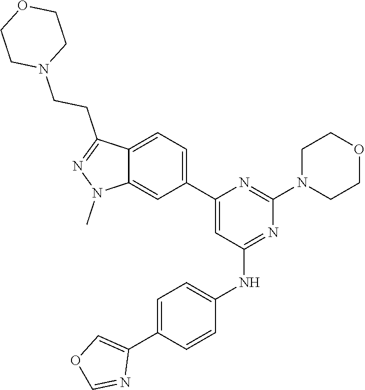 [SMILES: Cn1nc(CCN2CCOCC2)c2ccc(-c3cc(Nc4ccc(-c5cocn5)cc4)nc(N4CCOCC4)n3)cc21] | {6-[1-methyl-3-(2-morpholin-4- ylethyl)(1H-indazol-6-yl)]-2-morpholin- 4-ylpyrimidin-4-yl}(4-(1,3-oxazol-4- yl)phenyl)amine |
| 191 | 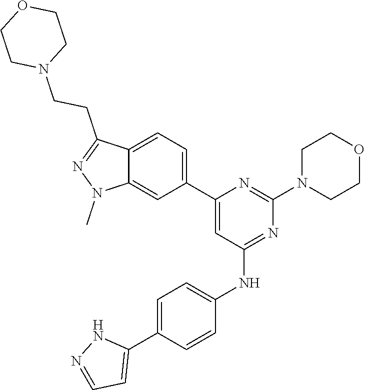 [SMILES: Cn1nc(CCN2CCOCC2)c2ccc(-c3cc(Nc4ccc(-c5ccn[nH]5)cc4)nc(N4CCOCC4)n3)cc21] | {6-[1-methyl-3-(2-morpholin-4- ylethyl)(1H-indazol-6-yl)]-2-morpholin- 4-ylpyrimidin-4-yl}(4-pyrazol-5- ylphenyl)amine |
| 192 | 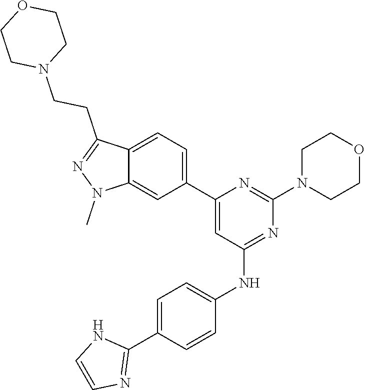 [SMILES: Cn1nc(CCN2CCOCC2)c2ccc(-c3cc(Nc4ccc(-c5ncc[nH]5)cc4)nc(N4CCOCC4)n3)cc21] | (4-imidazol-2-ylphenyl){6-[1-methyl-3- (2-morpholin-4-ylethyl)(1H-indazol-6- yl)]-2-morpholin-4-ylpyrimidin-4- yl}amine |
| 193 | 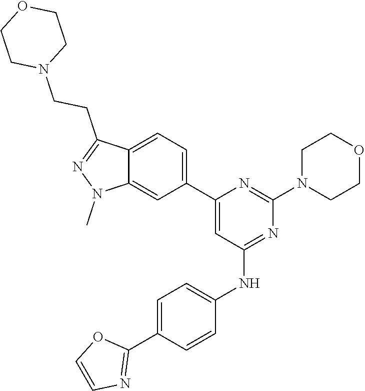 [SMILES: Cn1nc(CCN2CCOCC2)c2ccc(-c3cc(Nc4ccc(-c5ncco5)cc4)nc(N4CCOCC4)n3)cc21] | {6-[1-methyl-3-(2-morpholin-4- ylethyl)(1H-indazol-6-yl)]-2-morpholin- 4-ylpyrimidin-4-yl}(4-(1,3-oxazol-2- yl)phenyl)amine |
| 194 | 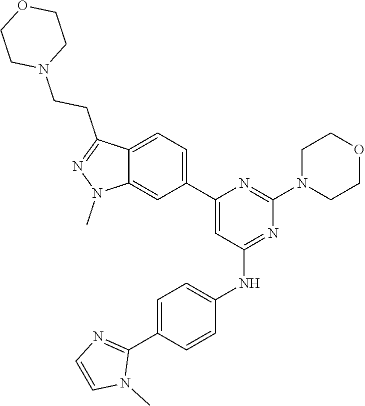 [SMILES: Cn1ccnc1-c1ccc(Nc2cc(-c3ccc4c(CCN5CCOCC5)nn(C)c4c3)nc(N3CCOCC3)n2)cc1] | [4-(1-methylimidazol-2-yl)phenyl]{6- [1-methyl-3-(2-morpholin-4- ylethyl)(1H-indazol-6-yl)]-2-morpholin- 4-ylpyrimidin-4-yl}amine |
| 195 | 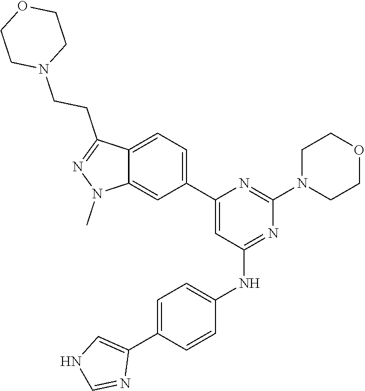 [SMILES: Cn1nc(CCN2CCOCC2)c2ccc(-c3cc(Nc4ccc(-c5c[nH]cn5)cc4)nc(N4CCOCC4)n3)cc21] | (4-imidazol-4-ylphenyl){6-[1-methyl-3- (2-morpholin-4-ylethyl)(1H-indazol-6- yl)]-2-morpholin-4-ylpyrimidin-4- yl}amine |
| 196 | 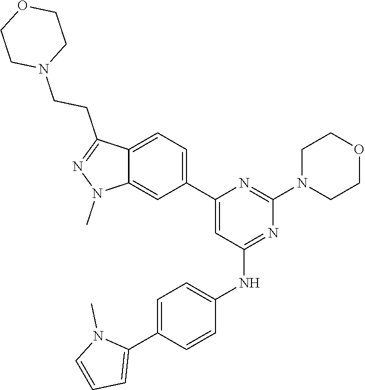 [SMILES: Cn1cccc1-c1ccc(Nc2cc(-c3ccc4c(CCN5CCOCC5)nn(C)c4c3)nc(N3CCOCC3)n2)cc1] | {6-[1-methyl-3-(2-morpholin-4- ylethyl)(1H-indazol-6-yl)]-2-morpholin- 4-ylpyrimidin-4-yl}[4-(1-methylpyrrol- 2-yl)phenyl]amine |
| 197 | 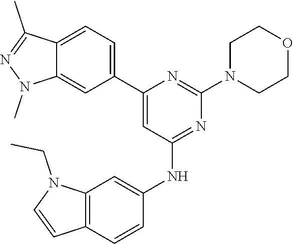 [SMILES: CCn1ccc2ccc(Nc3cc(-c4ccc5c(C)nn(C)c5c4)nc(N4CCOCC4)n3)cc21] | [6-(1,3-dimethyl(1H-indazol-6-yl))-2- morpholin-4-ylpyrimidin-4-yl](1- ethylindol-6-yl)amine |
| 198 | 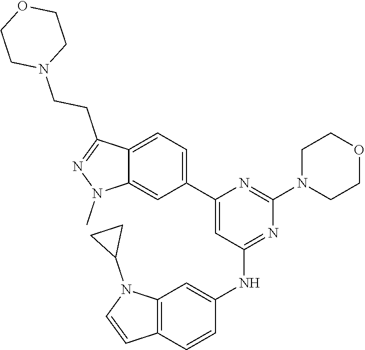 [SMILES: Cn1nc(CCN2CCOCC2)c2ccc(-c3cc(Nc4ccc5ccn(C6CC6)c5c4)nc(N4CCOCC4)n3)cc21] | (1-cyclopropylindol-6-yl){6-[1-methyl- 3-(2-morpholin-4-ylethyl)(1H-indazol- 6-yl)]-2-morpholin-4-ylpyrimidin-4- yl}amine |
| 199 | 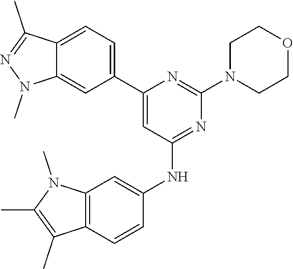 [SMILES: Cc1nn(C)c2cc(-c3cc(Nc4ccc5c(C)c(C)n(C)c5c4)nc(N4CCOCC4)n3)ccc12] | [6-(1,3-dimethyl(1H-indazol-6-yl))-2- morpholin-4-ylpyrimidin-4-yl](1,2,3- trimethylindol-6-yl)amine |
| 200 | 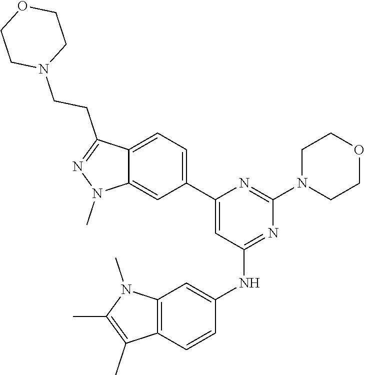 [SMILES: Cc1c(C)n(C)c2cc(Nc3cc(-c4ccc5c(CCN6CCOCC6)nn(C)c5c4)nc(N4CCOCC4)n3)ccc12] | {6-[1-methyl-3-(2-morpholin-4- ylethyl)(1H-indazol-6-yl)]-2-morpholin- 4-ylpyrimidin-4-yl}(1,2,3- trimethylindol-6-yl)amine |
| 201 | 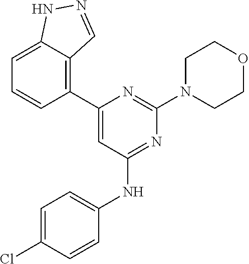 [SMILES: Clc1ccc(Nc2cc(-c3cccc4[nH]ncc34)nc(N3CCOCC3)n2)cc1] | (6-(1H-indazol-4-yl)-2-morpholin-4- ylpyrimidin-4-yl)(4-chlorophenyl)amine |
| 202 | 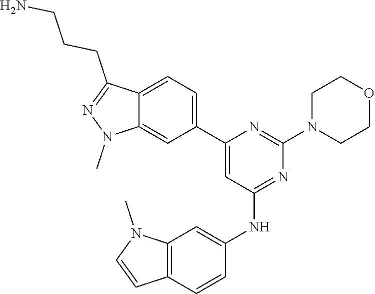 [SMILES: Cn1ccc2ccc(Nc3cc(-c4ccc5c(CCCN)nn(C)c5c4)nc(N4CCOCC4)n3)cc21] | {6-[3-(3-aminopropyl)-1-methyl(1H- indazol-6-yl)]-2-morpholin-4- ylpyrimidin-4-yl}(1-methylindol-6- yl)amine |
| 203 | 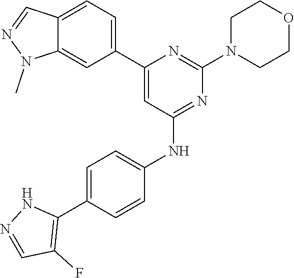 [SMILES: Cn1ncc2ccc(-c3cc(Nc4ccc(-c5[nH]ncc5F)cc4)nc(N4CCOCC4)n3)cc21] | [4-(4-fluoropyrazol-5-yl)phenyl][6-(1- methyl(1H-indazol-6-yl))-2-morpholin- 4-ylpyrimidin-4-yl]amine |
| 204 | 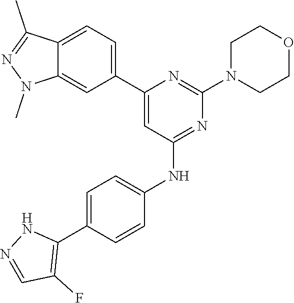 [SMILES: Cc1nn(C)c2cc(-c3cc(Nc4ccc(-c5[nH]ncc5F)cc4)nc(N4CCOCC4)n3)ccc12] | [6-(1,3-dimethyl(1H-indazol-6-yl))-2- morpholin-4-ylpyrimidin-4-yl][4-(4- fluoropyrazol-5-yl)phenyl]amine |
| 205 | 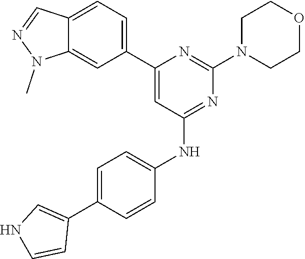 [SMILES: Cn1ncc2ccc(-c3cc(Nc4ccc(-c5cc[nH]c5)cc4)nc(N4CCOCC4)n3)cc21] | [6-(1-methyl(1H-indazol-6-yl))-2- morpholin-4-ylpyrimidin-4-yl](4-pyrrol- 3-ylphenyl)amine |
| 206 | 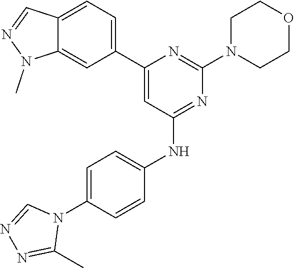 [SMILES: Cc1nncn1-c1ccc(Nc2cc(-c3ccc4cnn(C)c4c3)nc(N3CCOCC3)n2)cc1] | [4-(3-methyl(1,2,4-triazol-4- yl))phenyl][6-(1-methyl(1H-indazol-6- yl))-2-morpholin-4-ylpyrimidin-4- yl]amine |
| 207 | 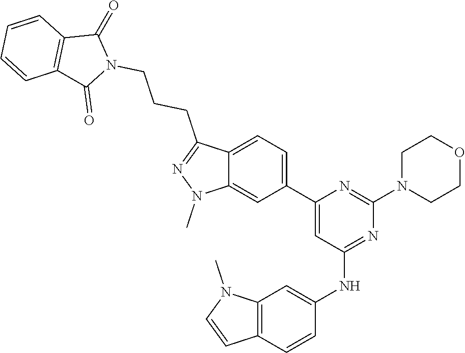 [SMILES: Cn1ccc2ccc(Nc3cc(-c4ccc5c(CCCN6C(=O)c7ccccc7C6=O)nn(C)c5c4)nc(N4CCOCC4)n3)cc21] | 2-[3-(1-methyl-6-{6-[(1-methylindol-6- yl)amino]-2-morpholin-4-ylpyrimidin-4- yl}-1H-indazol-3- yl)propyl]benzo[c]azoline-1,3-dione |
| 208 | 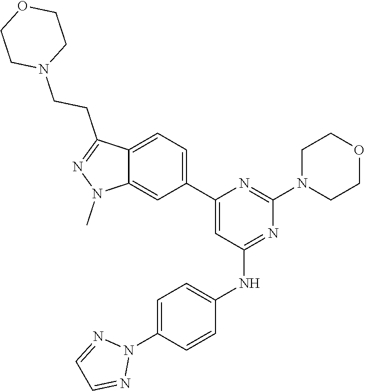 [SMILES: Cn1nc(CCN2CCOCC2)c2ccc(-c3cc(Nc4ccc(-n5nccn5)cc4)nc(N4CCOCC4)n3)cc21] | {6-[1-methyl-3-(2-morpholin-4- ylethyl)(1H-indazol-6-yl)]-2-morpholin- 4-ylpyrimidin-4-yl}(4-(1,2,3-triazol-2- yl)phenyl)amine |
| 209 | 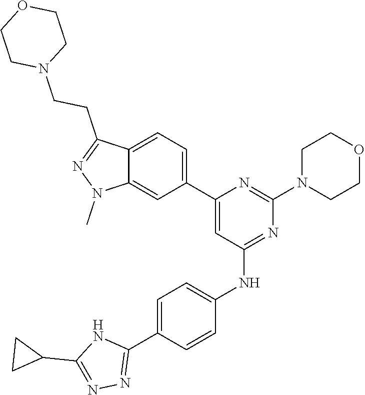 [SMILES: Cn1nc(CCN2CCOCC2)c2ccc(-c3cc(Nc4ccc(-c5nnc(C6CC6)[nH]5)cc4)nc(N4CCOCC4)n3)cc21] | [4-(5-cyclopropyl(4H-1,2,4-triazol-3- yl))phenyl]{6-[1-methyl-3-(2- morpholin-4-ylethyl)(1H-indazol-6-yl)]- 2-morpholin-4-ylpyrimidin-4-yl}amine |
| 210 | 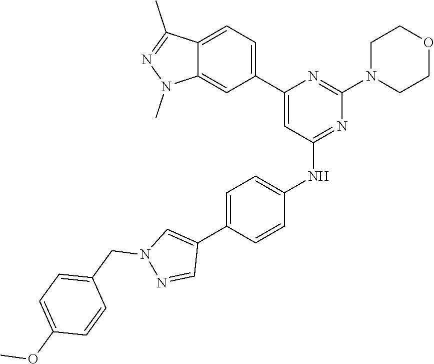 [SMILES: COc1ccc(Cn2cc(-c3ccc(Nc4cc(-c5ccc6c(C)nn(C)c6c5)nc(N5CCOCC5)n4)cc3)cn2)cc1] | [6-(1,3-dimethyl(1H-indazol-6-yl))-2- morpholin-4-ylpyrimidin-4-yl](4-{1-[(4- methoxyphenyl)methyl]pyrazol-4- yl}phenyl)amine |
| 211 | 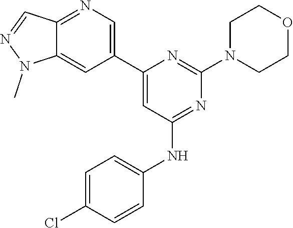 [SMILES: Cn1ncc2ncc(-c3cc(Nc4ccc(Cl)cc4)nc(N4CCOCC4)n3)cc21] | (4-chlorophenyl)[6-(1- methylpyrazolo[4,5-b]pyridin-6-yl)-2- morpholin-4-ylpyrimidin-4-yl]amine |
| 212 | 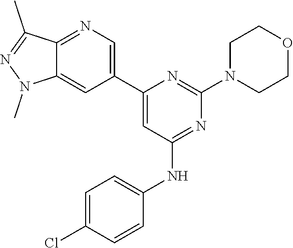 [SMILES: Cc1nn(C)c2cc(-c3cc(Nc4ccc(Cl)cc4)nc(N4CCOCC4)n3)cnc12] | [6-(1,3-dimethylpyrazolo[4,5-b]pyridin- 6-yl)-2-morpholin-4-ylpyrimidin-4- yl](4-chlorophenyl)amine |
| 213 | 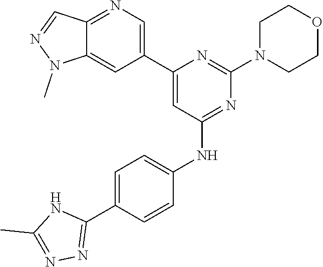 [SMILES: Cc1nnc(-c2ccc(Nc3cc(-c4cnc5cnn(C)c5c4)nc(N4CCOCC4)n3)cc2)[nH]1] | [4-(5-methyl(4H-1,2,4-triazol-3- yl))phenyl][6-(1-methylpyrazolo[4,5- b]pyridin-6-yl)-2-morpholin-4- ylpyrimidin-4-yl]amine |
| 214 | 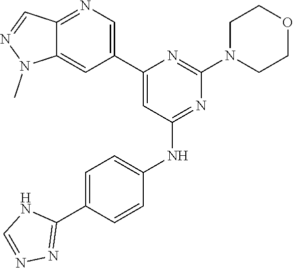 [SMILES: Cn1ncc2ncc(-c3cc(Nc4ccc(-c5nnc[nH]5)cc4)nc(N4CCOCC4)n3)cc21] | (4-(4H-1,2,4-triazol-3-yl)phenyl)[6-(1- methylpyrazolo[4,5-b]pyridin-6-yl)-2- morpholin-4-ylpyrimidin-4-yl]amine |
| 215 | 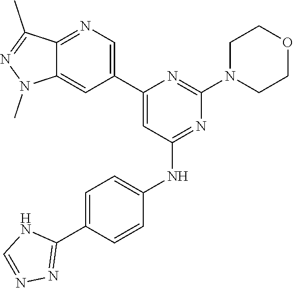 [SMILES: Cc1nn(C)c2cc(-c3cc(Nc4ccc(-c5nnc[nH]5)cc4)nc(N4CCOCC4)n3)cnc12] | (4-(4H-1,2,4-triazol-3-yl)phenyl)[6- (1,3-dimethylpyrazolo[4,5-b]pyridin-6- yl)-2-morpholin-4-ylpyrimidin-4- yl]amine |
| 216 | 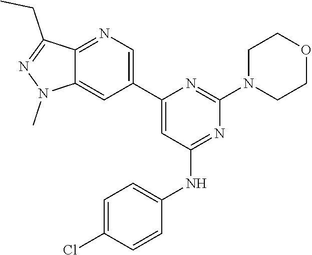 [SMILES: CCc1nn(C)c2cc(-c3cc(Nc4ccc(Cl)cc4)nc(N4CCOCC4)n3)cnc12] | (4-chlorophenyl)[6-(3-ethyl-1- methylpyrazolo[4,5-b]pyridin-6-yl)-2- morpholin-4-ylpyrimidin-4-yl]amine |
| 217 | 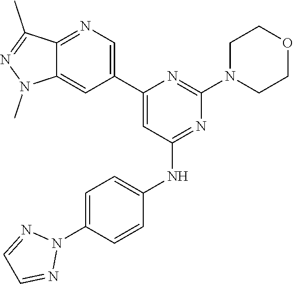 [SMILES: Cc1nn(C)c2cc(-c3cc(Nc4ccc(-n5nccn5)cc4)nc(N4CCOCC4)n3)cnc12] | [6-(1,3-dimethylpyrazolo[4,5-b]pyridin- 6-yl)-2-morpholin-4-ylpyrimidin-4- yl](4-(1,2,3-triazol-2-yl)phenyl)amine |
| 218 | 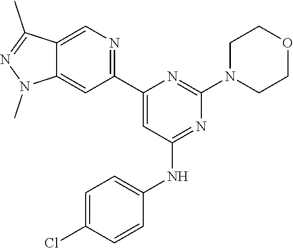 [SMILES: Cc1nn(C)c2cc(-c3cc(Nc4ccc(Cl)cc4)nc(N4CCOCC4)n3)ncc12] | [6-(1,3-dimethylpyrazolo[5,4-d]pyridin- 6-yl)-2-morpholin-4-ylpyrimidin-4- yl](4-chlorophenyl)amine |
| 219 | 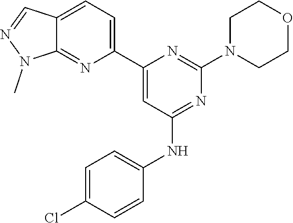 [SMILES: Cn1ncc2ccc(-c3cc(Nc4ccc(Cl)cc4)nc(N4CCOCC4)n3)nc21] | (4-chlorophenyl)[6-(1- methylpyrazolo[4,5-e]pyridin-6-yl)-2- morpholin-4-ylpyrimidin-4-yl]amine |
| 220 | 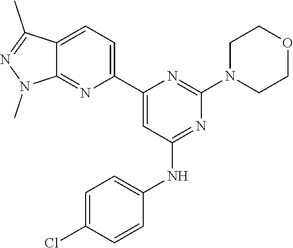 [SMILES: Cc1nn(C)c2nc(-c3cc(Nc4ccc(Cl)cc4)nc(N4CCOCC4)n3)ccc12] | [6-(1,3-dimethylpyrazolo[4,5-e]pyridin- 6-yl)-2-morpholin-4-ylpyrimidin-4- yl](4-chlorophenyl)amine |
| 221 | 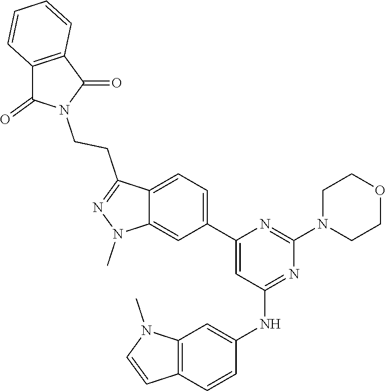 [SMILES: Cn1ccc2ccc(Nc3cc(-c4ccc5c(CCN6C(=O)c7ccccc7C6=O)nn(C)c5c4)nc(N4CCOCC4)n3)cc21] | 2-[2-(1-methyl-6-{6-[(1-methylindol-6- yl)amino]-2-morpholin-4-ylpyrimidin-4- yl}-1H-indazol-3- yl)ethyl]benzo[c]azoline-1,3-dione |
| 222 | 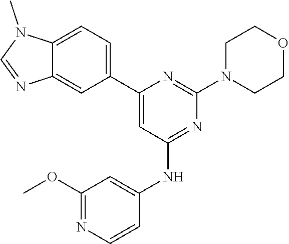 [SMILES: COc1cc(Nc2cc(-c3ccc4c(c3)ncn4C)nc(N3CCOCC3)n2)ccn1] | (2-methoxy(4-pyridyl))[6-(1- methylbenzimidazol-5-yl)-2-morpholin- 4-ylpyrimidin-4-yl]amine |
| 223 | 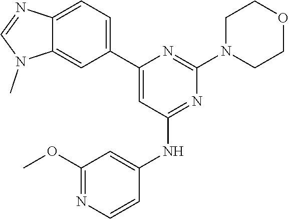 [SMILES: COc1cc(Nc2cc(-c3ccc4ncn(C)c4c3)nc(N3CCOCC3)n2)ccn1] | (2-methoxy(4-pyridyl))[6-(1- methylbenzimidazol-6-yl)-2-morpholin- 4-ylpyrimidin-4-yl]amine |
| 224 | 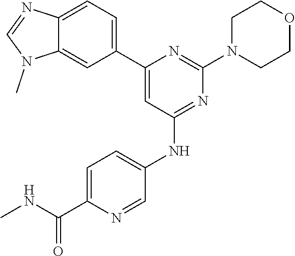 [SMILES: CNC(=O)c1ccc(Nc2cc(-c3ccc4ncn(C)c4c3)nc(N3CCOCC3)n2)cn1] | N-methyl(5-{[6-(1-methylbenzimidazol- 6-yl)-2-morpholin-4-ylpyrimidin-4- yl]amino}(2-pyridyl))carboxamide |
| 225 | 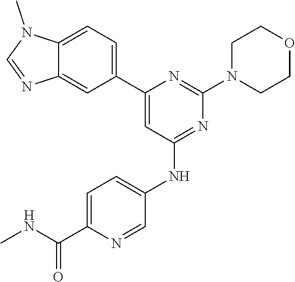 [SMILES: CNC(=O)c1ccc(Nc2cc(-c3ccc4c(c3)ncn4C)nc(N3CCOCC3)n2)cn1] | N-methyl(5-{[6-(1-methylbenzimidazol- 5-yl)-2-morpholin-4-ylpyrimidin-4- yl]amino}(2-pyridyl))carboxamide |
| 226 | 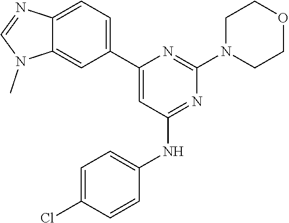 [SMILES: Cn1cnc2ccc(-c3cc(Nc4ccc(Cl)cc4)nc(N4CCOCC4)n3)cc21] | (4-chlorophenyl)[6-(1- methylbenzimidazol-6-yl)-2-morpholin- 4-ylpyrimidin-4-yl]amine |
| 227 | 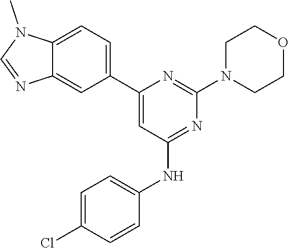 [SMILES: Cn1cnc2cc(-c3cc(Nc4ccc(Cl)cc4)nc(N4CCOCC4)n3)ccc21] | (4-chlorophenyl)[6-(1- methylbenzimidazol-5-yl)-2-morpholin- 4-ylpyrimidin-4-yl]amine |
| 228 | 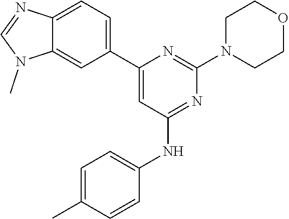 [SMILES: Cc1ccc(Nc2cc(-c3ccc4ncn(C)c4c3)nc(N3CCOCC3)n2)cc1] | [6-(1-methylbenzimidazol-6-yl)-2- morpholin-4-ylpyrimidin-4-yl](4- methylphenyl)amine |
| 229 | 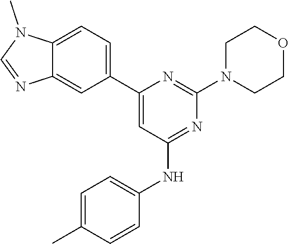 [SMILES: Cc1ccc(Nc2cc(-c3ccc4c(c3)ncn4C)nc(N3CCOCC3)n2)cc1] | [6-(1-methylbenzimidazol-5-yl)-2- morpholin-4-ylpyrimidin-4-yl](4- methylphenyl)amine |
| 230 | 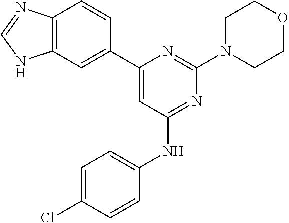 [SMILES: Clc1ccc(Nc2cc(-c3ccc4nc[nH]c4c3)nc(N3CCOCC3)n2)cc1] | (6-benzimidazol-6-yl-2-morpholin-4- ylpyrimidin-4-yl)(4-chlorophenyl)amine |
| 231 | 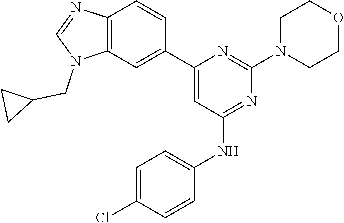 [SMILES: Clc1ccc(Nc2cc(-c3ccc4ncn(CC5CC5)c4c3)nc(N3CCOCC3)n2)cc1] | (4-chlorophenyl){6-[1- (cyclopropylmethyl)benzimidazol-6-yl]- 2-morpholin-4-ylpyrimidin-4-yl}amine |
| 232 | 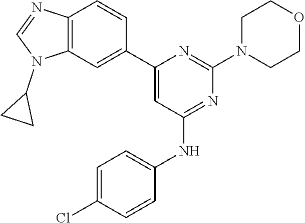 [SMILES: Clc1ccc(Nc2cc(-c3ccc4ncn(C5CC5)c4c3)nc(N3CCOCC3)n2)cc1] | (4-chlorophenyl)[6-(1- cyclopropylbenzimidazol-6-yl)-2- morpholin-4-ylpyrimidin-4-yl]amine |
| 233 | 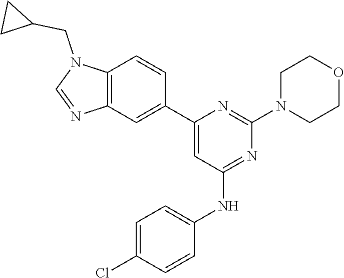 [SMILES: Clc1ccc(Nc2cc(-c3ccc4c(c3)ncn4CC3CC3)nc(N3CCOCC3)n2)cc1] | (4-chlorophenyl){6-[1- (cyclopropylmethyl)benzimidazol-5-yl]- 2-morpholin-4-ylpyrimidin-4-yl}amine |
| 234 | 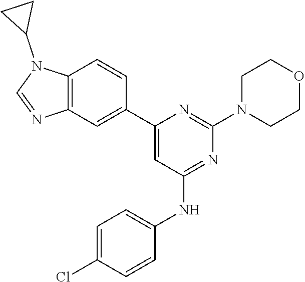 [SMILES: Clc1ccc(Nc2cc(-c3ccc4c(c3)ncn4C3CC3)nc(N3CCOCC3)n2)cc1] | (4-chlorophenyl)[6-(1- cyclopropylbenzimidazol-5-yl)-2- morpholin-4-ylpyrimidin-4-yl]amine |
| 235 | 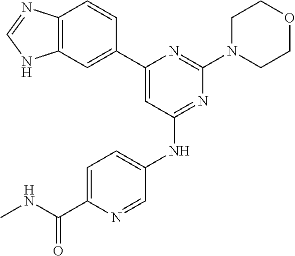 [SMILES: CNC(=O)c1ccc(Nc2cc(-c3ccc4nc[nH]c4c3)nc(N3CCOCC3)n2)cn1] | {5-[(6-benzimidazol-6-yl-2-morpholin- 4-ylpyrimidin-4-yl)amino](2-pyridyl)}- N-methylcarboxamide |
| 236 | 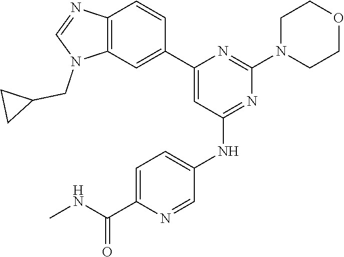 [SMILES: CNC(=O)c1ccc(Nc2cc(-c3ccc4ncn(CC5CC5)c4c3)nc(N3CCOCC3)n2)cn1] | [5-({6-[1- (cyclopropylmethyl)benzimidazol-6-yl]- 2-morpholin-4-ylpyrimidin-4- yl}amino)(2-pyridyl)]-N- methylcarboxamide |
| 237 | 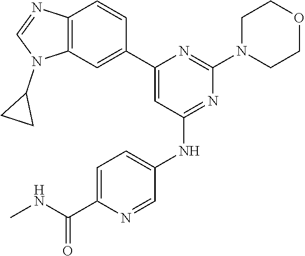 [SMILES: CNC(=O)c1ccc(Nc2cc(-c3ccc4ncn(C5CC5)c4c3)nc(N3CCOCC3)n2)cn1] | (5-{[6-(1-cyclopropylbenzimidazol-6- yl)-2-morpholin-4-ylpyrimidin-4- yl]amino}(2-pyridyl))-N- methylcarboxamide |
| 238 | 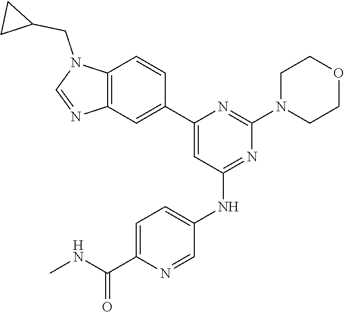 [SMILES: CNC(=O)c1ccc(Nc2cc(-c3ccc4c(c3)ncn4CC3CC3)nc(N3CCOCC3)n2)cn1] | [5-({6-[1- (cyclopropylmethyl)benzimidazol-5-yl]- 2-morpholin-4-ylpyrimidin-4- yl}amino)(2-pyridyl)]-N- methylcarboxamide |
| 239 | 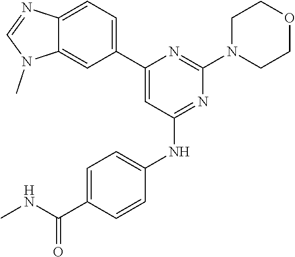 [SMILES: CNC(=O)c1ccc(Nc2cc(-c3ccc4ncn(C)c4c3)nc(N3CCOCC3)n2)cc1] | N-methyl(4-{[6-(1-methylbenzimidazol- 6-yl)-2-morpholin-4-ylpyrimidin-4- yl]amino}phenyl)carboxamide |
| 240 | 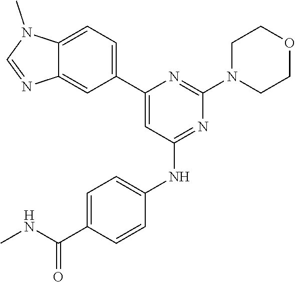 [SMILES: CNC(=O)c1ccc(Nc2cc(-c3ccc4c(c3)ncn4C)nc(N3CCOCC3)n2)cc1] | N-methyl(4-{[6-(1-methylbenzimidazol- 5-yl)-2-morpholin-4-ylpyrimidin-4- yl]amino}phenyl)carboxamide |
| 241 | 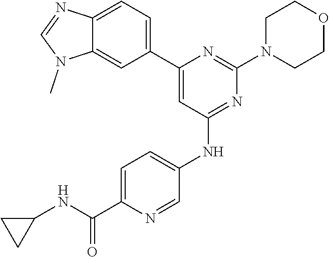 [SMILES: Cn1cnc2ccc(-c3cc(Nc4ccc(C(=O)NC5CC5)nc4)nc(N4CCOCC4)n3)cc21] | N-cyclopropyl(5-{[6-(1- methylbenzimidazol-6-yl)-2-morpholin- 4-ylpyrimidin-4-yl]amino}(2- pyridyl))carboxamide |
| 242 | 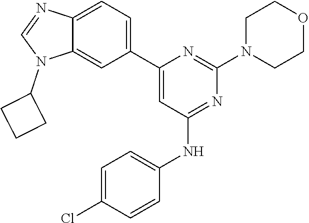 [SMILES: Clc1ccc(Nc2cc(-c3ccc4ncn(C5CCC5)c4c3)nc(N3CCOCC3)n2)cc1] | (4-chlorophenyl)[6-(1- cyclobutylbenzimidazol-6-yl)-2- morpholin-4-ylpyrimidin-4-yl]amine |
| 243 | 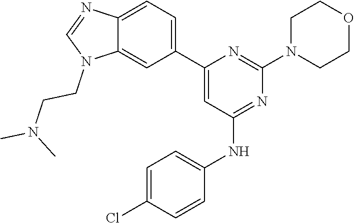 [SMILES: CN(C)CCn1cnc2ccc(-c3cc(Nc4ccc(Cl)cc4)nc(N4CCOCC4)n3)cc21] | [2-(6-{6-[(4-chlorophenyl)amino]-2- morpholin-4-ylpyrimidin-4- yl}benzimidazolyl)ethyl]dimethylamine |
| 244 | 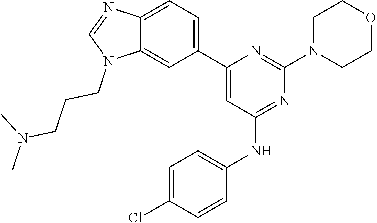 [SMILES: CN(C)CCCn1cnc2ccc(-c3cc(Nc4ccc(Cl)cc4)nc(N4CCOCC4)n3)cc21] | [3-(6-{6-[(4-chlorophenyl)amino]-2- morpholin-4-ylpyrimidin-4- yl}benzimidazolyl)propyl]dimethylamine |
| 245 | 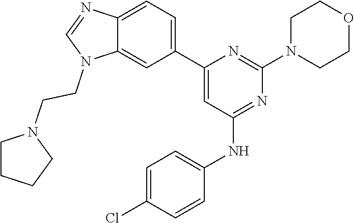 [SMILES: Clc1ccc(Nc2cc(-c3ccc4ncn(CCN5CCCC5)c4c3)nc(N3CCOCC3)n2)cc1] | (4-chlorophenyl){2-morpholin-4-yl-6- [1-(2-pyrrolidinylethyl)benzimidazol-6- yl]pyrimidin-4-yl}amine |
| 246 | 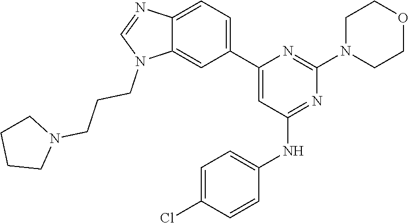 [SMILES: Clc1ccc(Nc2cc(-c3ccc4ncn(CCCN5CCCC5)c4c3)nc(N3CCOCC3)n2)cc1] | (4-chlorophenyl){2-morpholin-4-yl-6- [1-(3-pyrrolidinylpropyl)benzimidazol- 6-yl]pyrimidin-4-yl}amine |
| 247 | 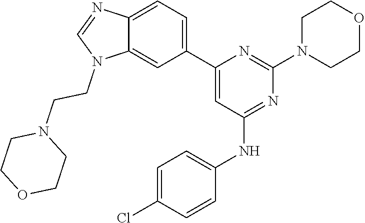 [SMILES: Clc1ccc(Nc2cc(-c3ccc4ncn(CCN5CCOCC5)c4c3)nc(N3CCOCC3)n2)cc1] | (4-chlorophenyl){2-morpholin-4-yl-6- [1-(2-morpholin-4- ylethyl)benzimidazol-6-yl]pyrimidin-4- yl}amine |
| 248 | 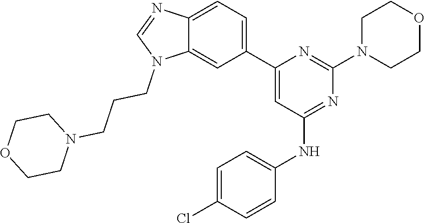 [SMILES: Clc1ccc(Nc2cc(-c3ccc4ncn(CCCN5CCOCC5)c4c3)nc(N3CCOCC3)n2)cc1] | (4-chlorophenyl){2-morpholin-4-yl-6- [1-(3-morpholin-4- ylpropyl)benzimidazol-6-yl]pyrimidin- 4-yl}amine |
| 249 | 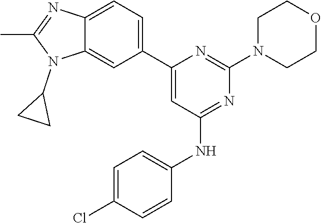 [SMILES: Cc1nc2ccc(-c3cc(Nc4ccc(Cl)cc4)nc(N4CCOCC4)n3)cc2n1C1CC1] | (4-chlorophenyl)[6-(1-cyclopropyl-2- methylbenzimidazol-6-yl)-2-morpholin- 4-ylpyrimidin-4-yl]amine |
| 250 | 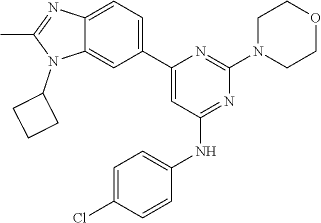 [SMILES: Cc1nc2ccc(-c3cc(Nc4ccc(Cl)cc4)nc(N4CCOCC4)n3)cc2n1C1CCC1] | (4-chlorophenyl)[6-(1-cyclobutyl-2- methylbenzimidazol-6-yl)-2-morpholin- 4-ylpyrimidin-4-yl]amine |
| 251 | 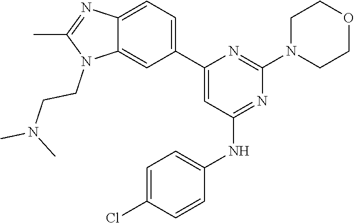 [SMILES: Cc1nc2ccc(-c3cc(Nc4ccc(Cl)cc4)nc(N4CCOCC4)n3)cc2n1CCN(C)C] | [2-(6-{6-[(4-chlorophenyl)amino]-2- morpholin-4-ylpyrimidin-4-yl}-2- methylbenzimidazolyl)ethyl]dimethyl- amine |
| 252 | 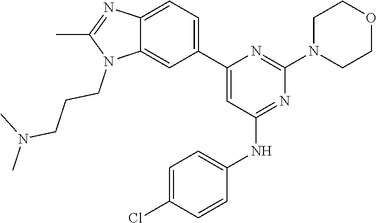 [SMILES: Cc1nc2ccc(-c3cc(Nc4ccc(Cl)cc4)nc(N4CCOCC4)n3)cc2n1CCCN(C)C] | [3-(6-{6-[(4-chlorophenyl)amino]-2- morpholin-4-ylpyrimidin-4-yl}-2- methylbenzimidazolyl)propyl]dimethyl- amine |
| 253 | 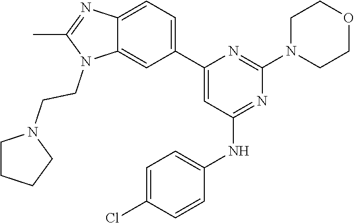 [SMILES: Cc1nc2ccc(-c3cc(Nc4ccc(Cl)cc4)nc(N4CCOCC4)n3)cc2n1CCN1CCCC1] | (4-chlorophenyl){6-[2-methyl-1-(2- pyrrolidinylethyl)benzimidazol-6-yl]-2- morpholin-4-ylpyrimidin-4-yl}amine |
| 254 | 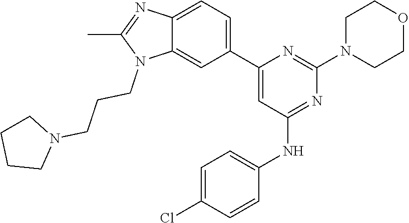 [SMILES: Cc1nc2ccc(-c3cc(Nc4ccc(Cl)cc4)nc(N4CCOCC4)n3)cc2n1CCCN1CCCC1] | (4-chlorophenyl){6-[2-methyl-1-(3- pyrrolidinylpropyl)benzimidazol-6-yl]- 2-morpholin-4-ylpyrimidin-4-yl}amine |
| 255 | 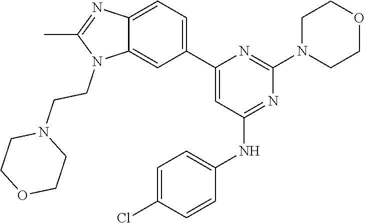 [SMILES: Cc1nc2ccc(-c3cc(Nc4ccc(Cl)cc4)nc(N4CCOCC4)n3)cc2n1CCN1CCOCC1] | (4-chlorophenyl){6-[2-methyl-1-(2- morpholin-4-ylethyl)benzimidazol-6- yl]-2-morpholin-4-ylpyrimidin-4- yl}amine |
| 256 | 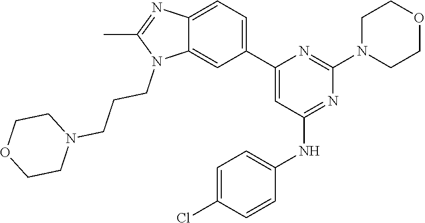 [SMILES: Cc1nc2ccc(-c3cc(Nc4ccc(Cl)cc4)nc(N4CCOCC4)n3)cc2n1CCCN1CCOCC1] | (4-chlorophenyl){6-[2-methyl-1-(3- morpholin-4-ylpropyl)benzimidazol-6- yl]-2-morpholin-4-ylpyrimidin-4- yl}amine |
| 257 | 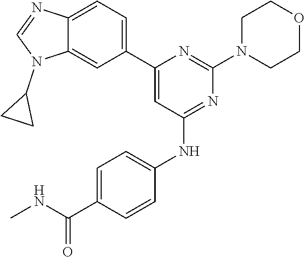 [SMILES: CNC(=O)c1ccc(Nc2cc(-c3ccc4ncn(C5CC5)c4c3)nc(N3CCOCC3)n2)cc1] | (4-{[6-(1-cyclopropylbenzimidazol-6- yl)-2-morpholin-4-ylpyrimidin-4- yl]amino}phenyl)-N- methylcarboxamide |
| 258 | 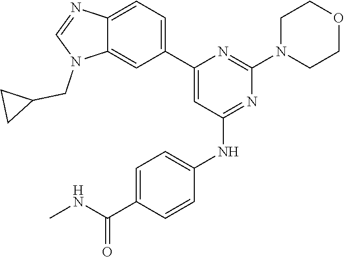 [SMILES: CNC(=O)c1ccc(Nc2cc(-c3ccc4ncn(CC5CC5)c4c3)nc(N3CCOCC3)n2)cc1] | [4-({6-[1- (cyclopropylmethyl)benzimidazol-6-yl]- 2-morpholin-4-ylpyrimidin-4- yl}amino)phenyl]-N- methylcarboxamide |
| 259 | 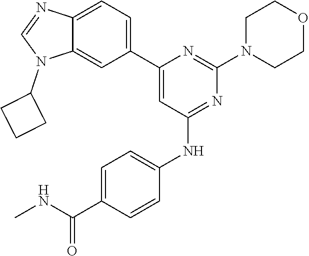 [SMILES: CNC(=O)c1ccc(Nc2cc(-c3ccc4ncn(C5CCC5)c4c3)nc(N3CCOCC3)n2)cc1] | (4-{[6-(1-cyclobutylbenzimidazol-6-yl)- 2-morpholin-4-ylpyrimidin-4- yl]amino}phenyl)-N- methylcarboxamide |
| 260 | 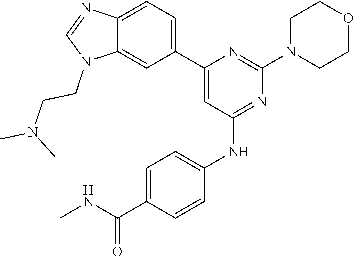 [SMILES: CNC(=O)c1ccc(Nc2cc(-c3ccc4ncn(CCN(C)C)c4c3)nc(N3CCOCC3)n2)cc1] | {4-[(6-{1-[2- (dimethylamino)ethyl]benzimidazol-6- yl}-2-morpholin-4-ylpyrimidin-4- yl)amino]phenyl}-N- methylcarboxamide |
| 261 | 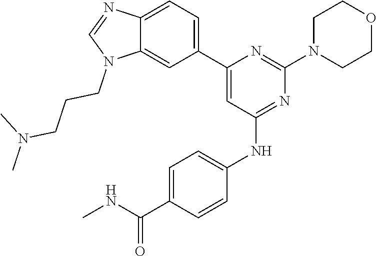 [SMILES: CNC(=O)c1ccc(Nc2cc(-c3ccc4ncn(CCCN(C)C)c4c3)nc(N3CCOCC3)n2)cc1] | {4-[(6-{1-[3- (dimethylamino)propyl]benzimidazol-6- yl}-2-morpholin-4-ylpyrimidin-4- yl)amino]phenyl}-N- methylcarboxamide |
| 262 | 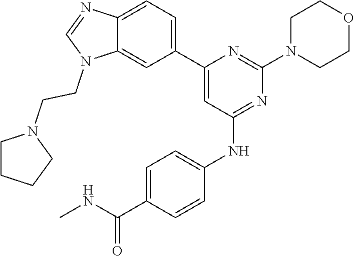 [SMILES: CNC(=O)c1ccc(Nc2cc(-c3ccc4ncn(CCN5CCCC5)c4c3)nc(N3CCOCC3)n2)cc1] | N-methyl[4-({2-morpholin-4-yl-6-[1-(2- pyrrolidinylethyl)benzimidazol-6- yl]pyrimidin-4- yl}amino)phenyl]carboxamide |
| 263 | 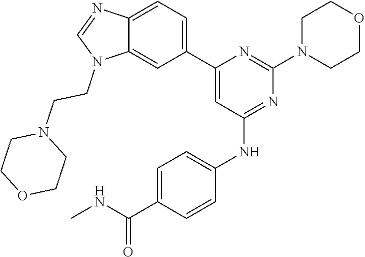 [SMILES: CNC(=O)c1ccc(Nc2cc(-c3ccc4ncn(CCN5CCOCC5)c4c3)nc(N3CCOCC3)n2)cc1] | N-methyl[4-({2-morpholin-4-yl-6-[1-(2- morpholin-4-ylethyl)benzimidazol-6- yl]pyrimidin-4- yl}amino)phenyl]carboxamide |
| 264 | 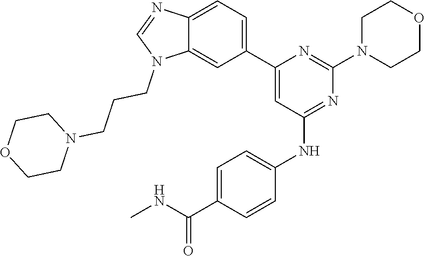 [SMILES: CNC(=O)c1ccc(Nc2cc(-c3ccc4ncn(CCCN5CCOCC5)c4c3)nc(N3CCOCC3)n2)cc1] | N-methyl[4-({2-morpholin-4-yl-6-[1-(3- morpholin-4-ylpropyl)benzimidazol-6- yl]pyrimidin-4- yl}amino)phenyl]carboxamide |
| 265 | 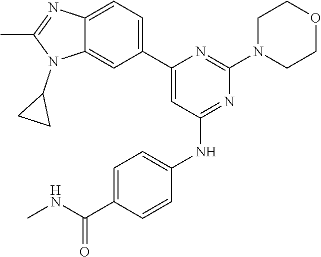 [SMILES: CNC(=O)c1ccc(Nc2cc(-c3ccc4nc(C)n(C5CC5)c4c3)nc(N3CCOCC3)n2)cc1] | (4-{[6-(1-cyclopropyl-2- methylbenzimidazol-6-yl)-2-morpholin- 4-ylpyrimidin-4-yl]amino}phenyl)-N- methylcarboxamide |
| 266 | 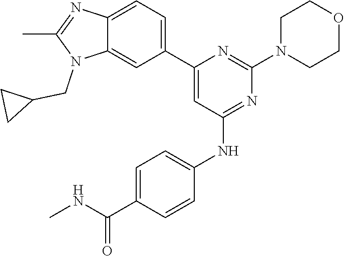 [SMILES: CNC(=O)c1ccc(Nc2cc(-c3ccc4nc(C)n(CC5CC5)c4c3)nc(N3CCOCC3)n2)cc1] | [4-({6-[1-(cyclopropylmethyl)-2- methylbenzimidazol-6-yl]-2-morpholin- 4-ylpyrimidin-4-yl}amino)phenyl]-N- methylcarboxamide |
| 267 | 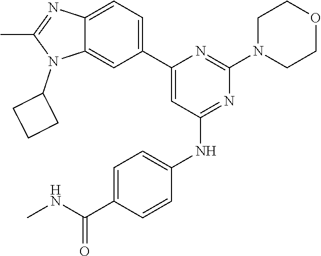 [SMILES: CNC(=O)c1ccc(Nc2cc(-c3ccc4nc(C)n(C5CCC5)c4c3)nc(N3CCOCC3)n2)cc1] | (4-{[6-(1-cyclobutyl-2- methylbenzimidazol-6-yl)-2-morpholin- 4-ylpyrimidin-4-yl]amino}phenyl)-N- methylcarboxamide |
| 268 | 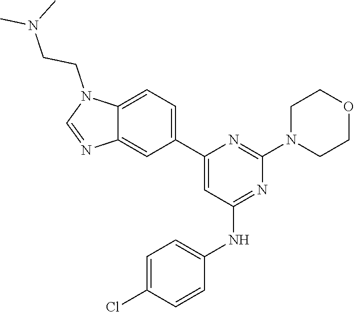 [SMILES: CN(C)CCn1cnc2cc(-c3cc(Nc4ccc(Cl)cc4)nc(N4CCOCC4)n3)ccc21] | [2-(5-{6-[(4-chlorophenyl)amino]-2- morpholin-4-ylpyrimidin-4- yl}benzimidazolyl)ethyl]dimethylamine |
| 269 | 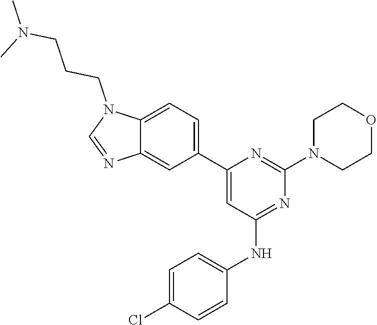 [SMILES: CN(C)CCCn1cnc2cc(-c3cc(Nc4ccc(Cl)cc4)nc(N4CCOCC4)n3)ccc21] | [3-(5-{6-[(4-chlorophenyl)amino]-2- morpholin-4-ylpyrimidin-4- yl}benzimidazolyl)propyl]dimethylamine |
| 270 | 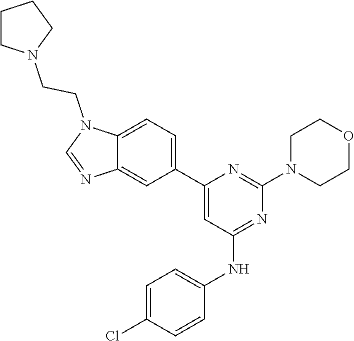 [SMILES: Clc1ccc(Nc2cc(-c3ccc4c(c3)ncn4CCN3CCCC3)nc(N3CCOCC3)n2)cc1] | (4-chlorophenyl){2-morpholin-4-yl-6- [1-(2-pyrrolidinylethyl)benzimidazol-5- yl]pyrimidin-4-yl}amine |
| 271 | 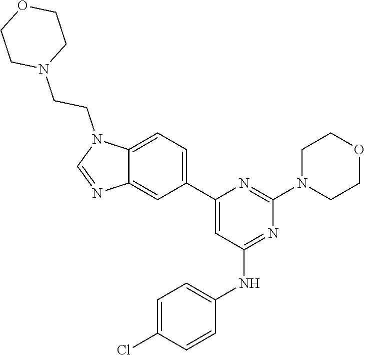 [SMILES: Clc1ccc(Nc2cc(-c3ccc4c(c3)ncn4CCN3CCOCC3)nc(N3CCOCC3)n2)cc1] | (4-chlorophenyl){2-morpholin-4-yl-6- [1-(2-morpholin-4- ylethyl)benzimidazol-5-yl]pyrimidin-4- yl}amine |
| 272 | 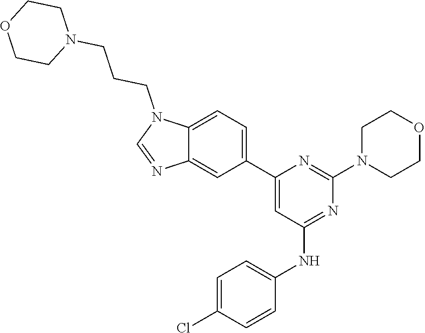 [SMILES: Clc1ccc(Nc2cc(-c3ccc4c(c3)ncn4CCCN3CCOCC3)nc(N3CCOCC3)n2)cc1] | (4-chlorophenyl){2-morpholin-4-yl-6- [1-(3-morpholin-4- ylpropyl)benzimidazol-5-yl]pyrimidin- 4-yl}amine |
| 273 | 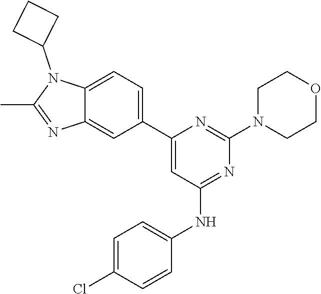 [SMILES: Cc1nc2cc(-c3cc(Nc4ccc(Cl)cc4)nc(N4CCOCC4)n3)ccc2n1C1CCC1] | (4-chlorophenyl)[6-(1-cyclobutyl-2- methylbenzimidazol-5-yl)-2-morpholin- 4-ylpyrimidin-4-yl]amine |
| 274 | 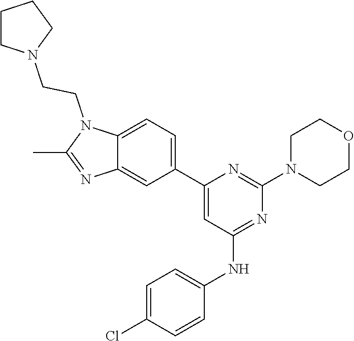 [SMILES: Cc1nc2cc(-c3cc(Nc4ccc(Cl)cc4)nc(N4CCOCC4)n3)ccc2n1CCN1CCCC1] | (4-chlorophenyl){6-[2-methyl-1-(2- pyrrolidinylethyl)benzimidazol-5-yl]-2- morpholin-4-ylpyrimidin-4-yl}amine |
| 275 | 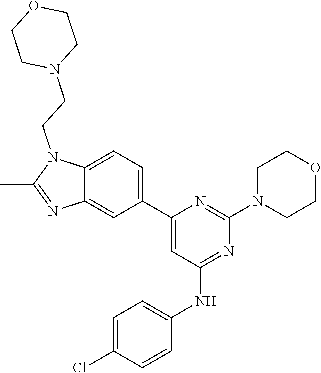 [SMILES: Cc1nc2cc(-c3cc(Nc4ccc(Cl)cc4)nc(N4CCOCC4)n3)ccc2n1CCN1CCOCC1] | (4-chlorophenyl){6-[2-methyl-1-(2- morpholin-4-ylethyl)benzimidazol-5- yl]-2-morpholin-4-ylpyrimidin-4- yl}amine |
| 276 | 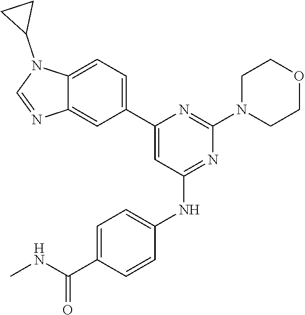 [SMILES: CNC(=O)c1ccc(Nc2cc(-c3ccc4c(c3)ncn4C3CC3)nc(N3CCOCC3)n2)cc1] | (4-{[6-(1-cyclopropylbenzimidazol-5- yl)-2-morpholin-4-ylpyrimidin-4- yl]amino}phenyl)-N- methylcarboxamide |
| 277 | 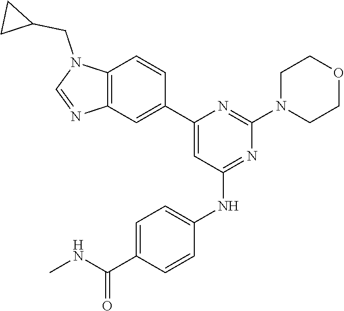 [SMILES: CNC(=O)c1ccc(Nc2cc(-c3ccc4c(c3)ncn4CC3CC3)nc(N3CCOCC3)n2)cc1] | [4-({6-[1- (cyclopropylmethyl)benzimidazol-5-yl]- 2-morpholin-4-ylpyrimidin-4- yl}amino)phenyl]-N- methylcarboxamide |
| 278 | 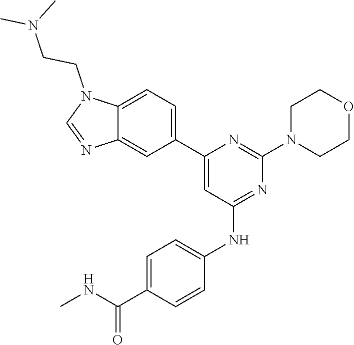 [SMILES: CNC(=O)c1ccc(Nc2cc(-c3ccc4c(c3)ncn4CCN(C)C)nc(N3CCOCC3)n2)cc1] | {4-[(6-{1-[2- (dimethylamino)ethyl]benzimidazol-5- yl}-2-morpholin-4-ylpyrimidin-4- yl)amino]phenyl}-N- methylcarboxamide |
| 279 | 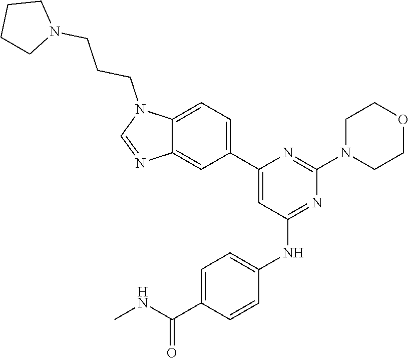 [SMILES: CNC(=O)c1ccc(Nc2cc(-c3ccc4c(c3)ncn4CCCN3CCCC3)nc(N3CCOCC3)n2)cc1] | N-methyl[4-({2-morpholin-4-yl-6-[1-(3- pyrrolidinylpropyl)benzimidazol-5- yl]pyrimidin-4- yl}amino)phenyl]carboxamide |
| 280 | 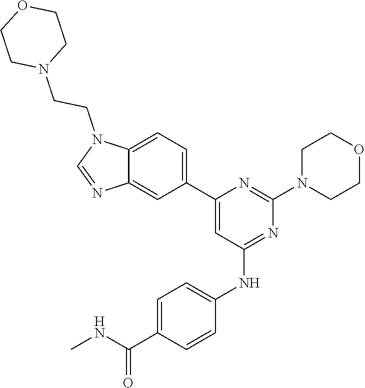 [SMILES: CNC(=O)c1ccc(Nc2cc(-c3ccc4c(c3)ncn4CCN3CCOCC3)nc(N3CCOCC3)n2)cc1] | N-methyl[4-({2-morpholin-4-yl-6-[1-(2- morpholin-4-ylethyl)benzimidazol-5- yl]pyrimidin-4- yl}amino)phenyl]carboxamide |
| 281 | 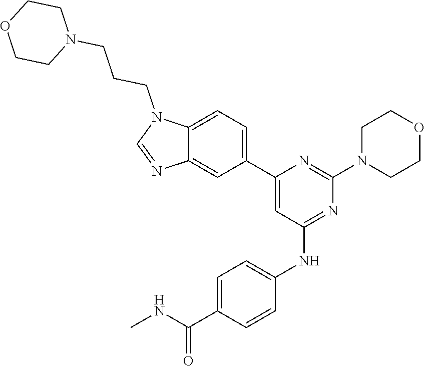 [SMILES: CNC(=O)c1ccc(Nc2cc(-c3ccc4c(c3)ncn4CCCN3CCOCC3)nc(N3CCOCC3)n2)cc1] | N-methyl[4-({2-morpholin-4-yl-6-[1-(3- morpholin-4-ylpropyl)benzimidazol-5- yl]pyrimidin-4- yl}amino)phenyl]carboxamide |
| 282 | 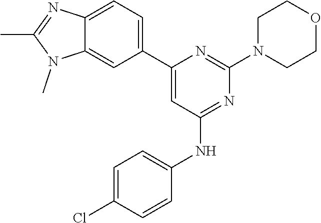 [SMILES: Cc1nc2ccc(-c3cc(Nc4ccc(Cl)cc4)nc(N4CCOCC4)n3)cc2n1C] | [6-(1,2-dimethylbenzimidazol-6-yl)-2- morpholin-4-ylpyrimidin-4-yl](4- chlorophenyl)amine |
| 283 | 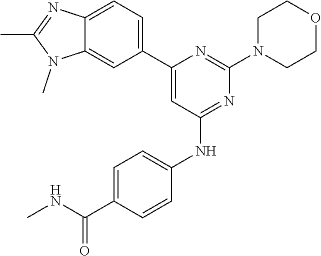 [SMILES: CNC(=O)c1ccc(Nc2cc(-c3ccc4nc(C)n(C)c4c3)nc(N3CCOCC3)n2)cc1] | (4-{[6-(1,2-dimethylbenzimidazol-6-yl)- 2-morpholin-4-ylpyrimidin-4- yl]amino}phenyl)-N- methylcarboxamide |
| 284 | 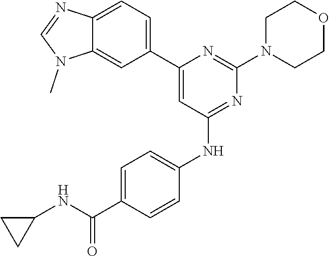 [SMILES: Cn1cnc2ccc(-c3cc(Nc4ccc(C(=O)NC5CC5)cc4)nc(N4CCOCC4)n3)cc21] | N-cyclopropyl(4-{[6-(1- methylbenzimidazol-6-yl)-2-morpholin- 4-ylpyrimidin-4- yl]amino}phenyl)carboxamide |
| 285 | 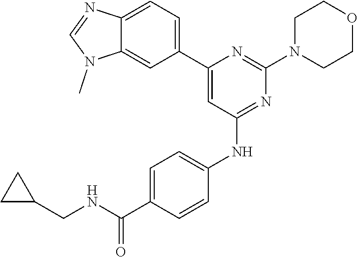 [SMILES: Cn1cnc2ccc(-c3cc(Nc4ccc(C(=O)NCC5CC5)cc4)nc(N4CCOCC4)n3)cc21] | N-(cyclopropylmethyl)(4-{[6-(1- methylbenzimidazol-6-yl)-2-morpholin- 4-ylpyrimidin-4- yl]amino}phenyl)carboxamide |
| 286 | 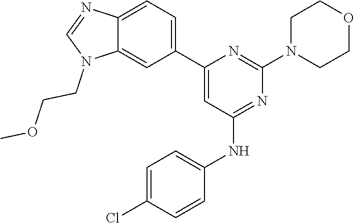 [SMILES: COCCn1cnc2ccc(-c3cc(Nc4ccc(Cl)cc4)nc(N4CCOCC4)n3)cc21] | (4-chlorophenyl){6-[1-(2- methoxyethyl)benzimidazol-6-yl]-2- morpholin-4-ylpyrimidin-4-yl}amine |
| 287 | 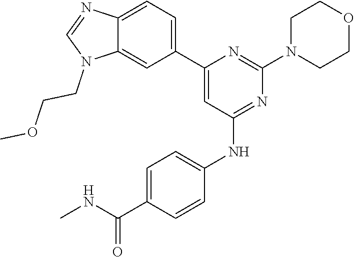 [SMILES: CNC(=O)c1ccc(Nc2cc(-c3ccc4ncn(CCOC)c4c3)nc(N3CCOCC3)n2)cc1] | [4-({6-[1-(2- methoxyethyl)benzimidazol-6-yl]-2- morpholin-4-ylpyrimidin-4- yl}amino)phenyl]-N- methylcarboxamide |
| 288 | 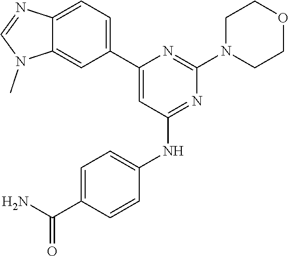 [SMILES: Cn1cnc2ccc(-c3cc(Nc4ccc(C(N)=O)cc4)nc(N4CCOCC4)n3)cc21] | 4-{[6-(1-methylbenzimidazol-6-yl)-2- morpholin-4-ylpyrimidin-4- yl]amino}benzamide |
| 289 | 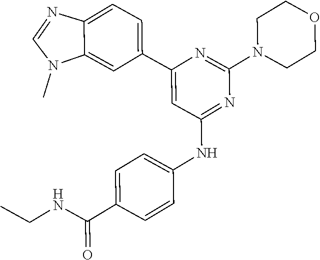 [SMILES: CCNC(=O)c1ccc(Nc2cc(-c3ccc4ncn(C)c4c3)nc(N3CCOCC3)n2)cc1] | N-ethyl(4-{[6-(1-methylbenzimidazol-6- yl)-2-morpholin-4-ylpyrimidin-4- yl]amino}phenyl)carboxamide |
| 290 | 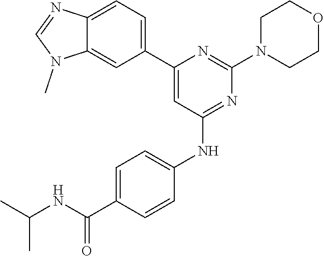 [SMILES: CC(C)NC(=O)c1ccc(Nc2cc(-c3ccc4ncn(C)c4c3)nc(N3CCOCC3)n2)cc1] | (4-{[6-(1-methylbenzimidazol-6-yl)-2- morpholin-4-ylpyrimidin-4- yl]amino}phenyl)-N- (methylethyl)carboxamide |
| 291 | 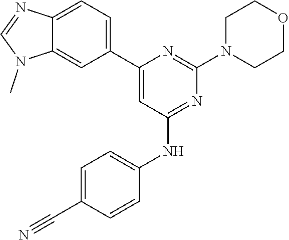 [SMILES: Cn1cnc2ccc(-c3cc(Nc4ccc(C#N)cc4)nc(N4CCOCC4)n3)cc21] | 4-{[6-(1-methylbenzimidazol-6-yl)-2- morpholin-4-ylpyrimidin-4- yl]amino}benzenecarbonitrile |
| 292 | 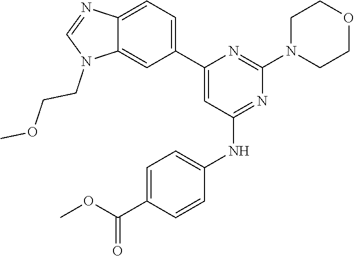 [SMILES: COCCn1cnc2ccc(-c3cc(Nc4ccc(C(=O)OC)cc4)nc(N4CCOCC4)n3)cc21] | methyl 4-({6-[1-(2- methoxyethyl)benzimidazol-6-yl]-2- morpholin-4-ylpyrimidin-4- yl}amino)benzoate |
| 293 | 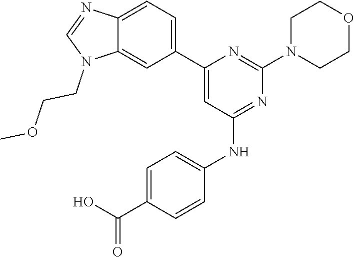 [SMILES: COCCn1cnc2ccc(-c3cc(Nc4ccc(C(=O)O)cc4)nc(N4CCOCC4)n3)cc21] | 4-({6-[1-(2- methoxyethyl)benzimidazol-6-yl]-2- morpholin-4-ylpyrimidin-4- yl}amino)benzoic acid |
| 294 | 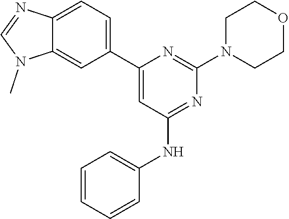 [SMILES: Cn1cnc2ccc(-c3cc(Nc4ccccc4)nc(N4CCOCC4)n3)cc21] | [6-(1-methylbenzimidazol-6-yl)-2- morpholin-4-ylpyrimidin-4- yl]phenylamine |
| 295 | 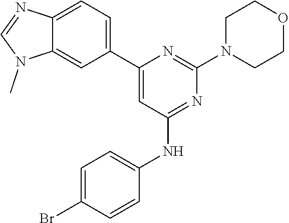 [SMILES: Cn1cnc2ccc(-c3cc(Nc4ccc(Br)cc4)nc(N4CCOCC4)n3)cc21] | (4-bromophenyl)[6-(1- methylbenzimidazol-6-yl)-2-morpholin- 4-ylpyrimidin-4-yl]amine |
| 296 | 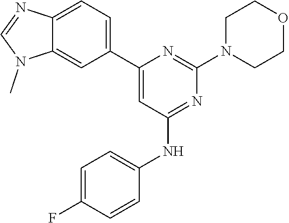 [SMILES: Cn1cnc2ccc(-c3cc(Nc4ccc(F)cc4)nc(N4CCOCC4)n3)cc21] | (4-fluorophenyl)[6-(1- methylbenzimidazol-6-yl)-2-morpholin- 4-ylpyrimidin-4-yl]amine |
| 297 | 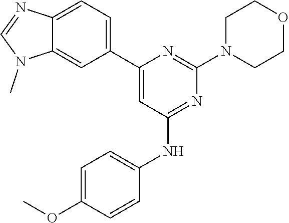 [SMILES: COc1ccc(Nc2cc(-c3ccc4ncn(C)c4c3)nc(N3CCOCC3)n2)cc1] | (4-methoxyphenyl)[6-(1- methylbenzimidazol-6-yl)-2-morpholin- 4-ylpyrimidin-4-yl]amine |
| 298 | 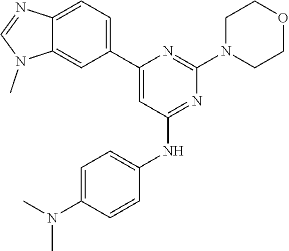 [SMILES: CN(C)c1ccc(Nc2cc(-c3ccc4ncn(C)c4c3)nc(N3CCOCC3)n2)cc1] | dimethyl(4-{[6-(1-methylbenzimidazol- 6-yl)-2-morpholin-4-ylpyrimidin-4- yl]amino}phenyl)amine |
| 299 | 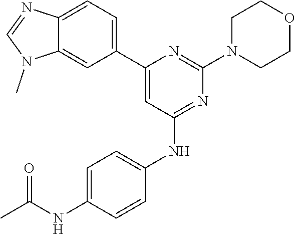 [SMILES: CC(=O)Nc1ccc(Nc2cc(-c3ccc4ncn(C)c4c3)nc(N3CCOCC3)n2)cc1] | N-(4-{[6-(1-methylbenzimidazol-6-yl)- 2-morpholin-4-ylpyrimidin-4- yl]amino}phenyl)acetamide |
| 300 | 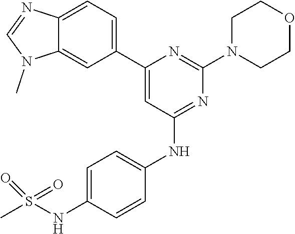 [SMILES: Cn1cnc2ccc(-c3cc(Nc4ccc(NS(C)(=O)=O)cc4)nc(N4CCOCC4)n3)cc21] | (4-{[6-(1-methylbenzimidazol-6-yl)-2- morpholin-4-ylpyrimidin-4- yl]amino}phenyl)(methylsulfonyl)amine |
| 301 | 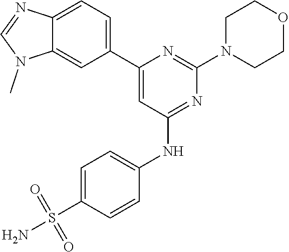 [SMILES: Cn1cnc2ccc(-c3cc(Nc4ccc(S(N)(=O)=O)cc4)nc(N4CCOCC4)n3)cc21] | 4-{[6-(1-methylbenzimidazol-6-yl)-2- morpholin-4-ylpyrimidin-4- yl]amino}benzenesulfonamide |
| 302 | 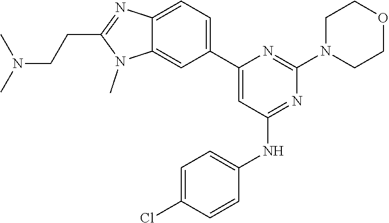 [SMILES: CN(C)CCc1nc2ccc(-c3cc(Nc4ccc(Cl)cc4)nc(N4CCOCC4)n3)cc2n1C] | [2-(6-{6-[(4-chlorophenyl)amino]-2- morpholin-4-ylpyrimidin-4-yl}-1- methylbenzimidazol-2- yl)ethyl]dimethylamine |
| 303 | 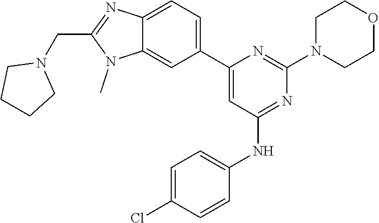 [SMILES: Cn1c(CN2CCCC2)nc2ccc(-c3cc(Nc4ccc(Cl)cc4)nc(N4CCOCC4)n3)cc21] | (4-chlorophenyl){6-[1-methyl-2- (pyrrolidinylmethyl)benzimidazol-6-yl]- 2-morpholin-4-ylpyrimidin-4-yl}amine |
| 304 | 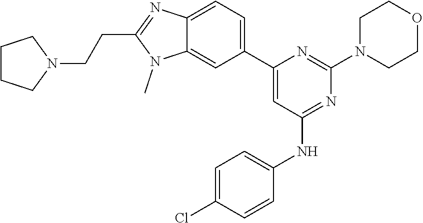 [SMILES: Cn1c(CCN2CCCC2)nc2ccc(-c3cc(Nc4ccc(Cl)cc4)nc(N4CCOCC4)n3)cc21] | (4-chlorophenyl){6-[1-methyl-2-(2- pyrrolidinylethyl)benzimidazol-6-yl]-2- morpholin-4-ylpyrimidin-4-yl}amine |
| 305 | 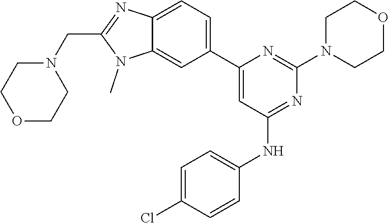 [SMILES: Cn1c(CN2CCOCC2)nc2ccc(-c3cc(Nc4ccc(Cl)cc4)nc(N4CCOCC4)n3)cc21] | (4-chlorophenyl){6-[1-methyl-2- (morpholin-4-ylmethyl)benzimidazol-6- yl]-2-morpholin-4-ylpyrimidin-4- yl}amine |
| 306 | 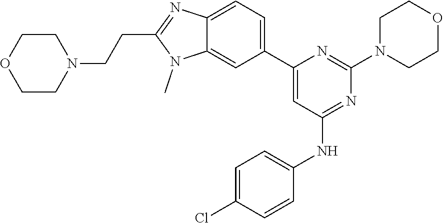 [SMILES: Cn1c(CCN2CCOCC2)nc2ccc(-c3cc(Nc4ccc(Cl)cc4)nc(N4CCOCC4)n3)cc21] | (4-chlorophenyl){6-[1-methyl-2-(2- morpholin-4-ylethyl)benzimidazol-6- yl]-2-morpholin-4-ylpyrimidin-4- yl}amine |
| 307 | 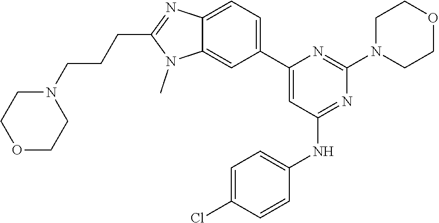 [SMILES: Cn1c(CCCN2CCOCC2)nc2ccc(-c3cc(Nc4ccc(Cl)cc4)nc(N4CCOCC4)n3)cc21] | (4-chlorophenyl){6-[1-methyl-2-(3- morpholin-4-ylpropyl)benzimidazol-6- yl]-2-morpholin-4-ylpyrimidin-4- yl}amine |
| 308 | 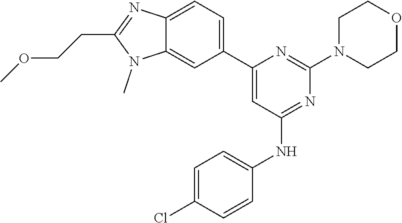 [SMILES: COCCc1nc2ccc(-c3cc(Nc4ccc(Cl)cc4)nc(N4CCOCC4)n3)cc2n1C] | (4-chlorophenyl){6-[2-(2- methoxyethyl)-1-methylbenzimidazol-6- yl]-2-morpholin-4-ylpyrimidin-4- yl}amine |
| 309 | 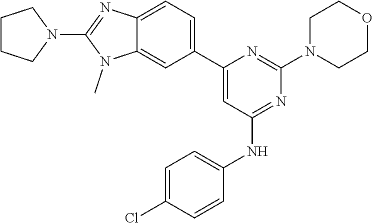 [SMILES: Cn1c(N2CCCC2)nc2ccc(-c3cc(Nc4ccc(Cl)cc4)nc(N4CCOCC4)n3)cc21] | (4-chlorophenyl)[6-(1-methyl-2- pyrrolidinylbenzimidazol-6-yl)-2- morpholin-4-ylpyrimidin-4-yl]amine |
| 310 | 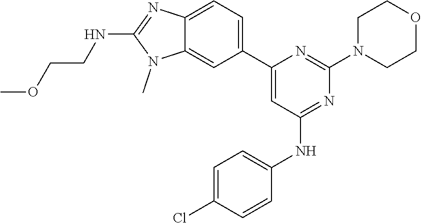 [SMILES: COCCNc1nc2ccc(-c3cc(Nc4ccc(Cl)cc4)nc(N4CCOCC4)n3)cc2n1C] | (4-chlorophenyl)(6-{2-[(2- methoxyethyl)amino]-1- methylbenzimidazol-6-yl}-2-morpholin- 4-ylpyrimidin-4-yl)amine |
| 311 | 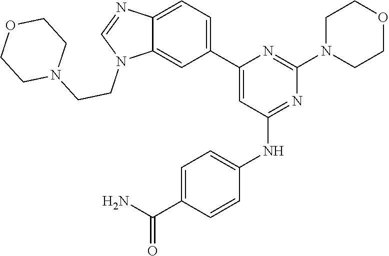 [SMILES: NC(=O)c1ccc(Nc2cc(-c3ccc4ncn(CCN5CCOCC5)c4c3)nc(N3CCOCC3)n2)cc1] | 4-({2-morpholin-4-yl-6-[1-(2- morpholin-4-ylethyl)benzimidazol-6- yl]pyrimidin-4-yl}amino)benzamide |
| 312 | 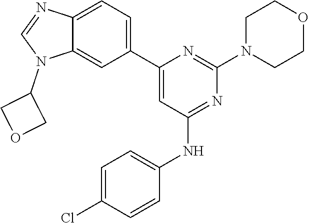 [SMILES: Clc1ccc(Nc2cc(-c3ccc4ncn(C5COC5)c4c3)nc(N3CCOCC3)n2)cc1] | (4-chlorophenyl)[2-morpholin-4-yl-6- (1-oxetan-3-ylbenzimidazol-6- yl)pyrimidin-4-yl]amine |
| 313 | 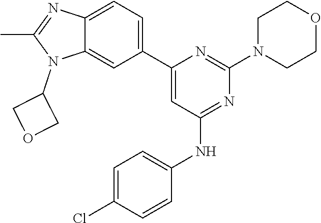 [SMILES: Cc1nc2ccc(-c3cc(Nc4ccc(Cl)cc4)nc(N4CCOCC4)n3)cc2n1C1COC1] | (4-chlorophenyl)[6-(2-methyl-1-oxetan- 3-ylbenzimidazol-6-yl)-2-morpholin-4- ylpyrimidin-4-yl]amine |
| 314 | 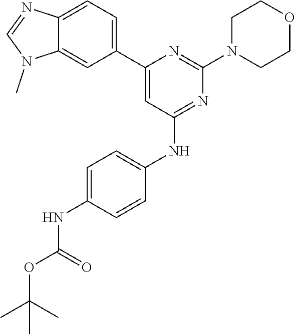 [SMILES: Cn1cnc2ccc(-c3cc(Nc4ccc(NC(=O)OC(C)(C)C)cc4)nc(N4CCOCC4)n3)cc21] | (tert-butoxy)-N-(4-{[6-(1- methylbenzimidazol-6-yl)-2-morpholin- 4-ylpyrimidin-4- yl]amino}phenyl)carboxamide |
| 315 | 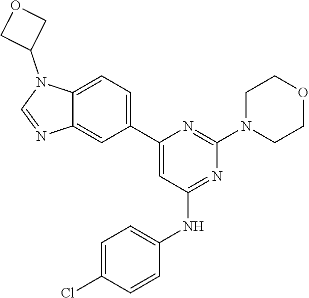 [SMILES: Clc1ccc(Nc2cc(-c3ccc4c(c3)ncn4C3COC3)nc(N3CCOCC3)n2)cc1] | (4-chlorophenyl)[2-morpholin-4-yl-6- (1-oxetan-3-ylbenzimidazol-5- yl)pyrimidin-4-yl]amine |
| 316 | 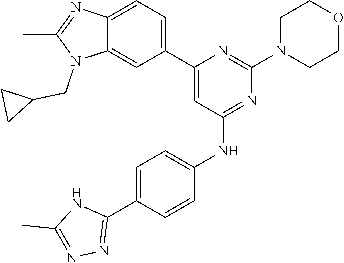 [SMILES: Cc1nnc(-c2ccc(Nc3cc(-c4ccc5nc(C)n(CC6CC6)c5c4)nc(N4CCOCC4)n3)cc2)[nH]1] | {6-[1-(cyclopropylmethyl)-2- methylbenzimidazol-6-yl]-2-morpholin- 4-ylpyrimidin-4-yl}[4-(5-methyl(4H- 1,2,4-triazol-3-yl))phenyl]amine |
| 317 | 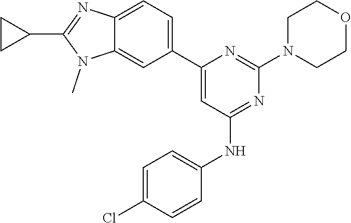 [SMILES: Cn1c(C2CC2)nc2ccc(-c3cc(Nc4ccc(Cl)cc4)nc(N4CCOCC4)n3)cc21] | (4-chlorophenyl)[6-(2-cyclopropyl-1- methylbenzimidazol-6-yl)-2-morpholin- 4-ylpyrimidin-4-yl]amine |
| 318 | 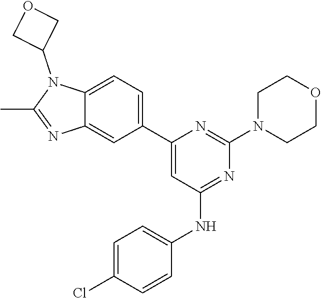 [SMILES: Cc1nc2cc(-c3cc(Nc4ccc(Cl)cc4)nc(N4CCOCC4)n3)ccc2n1C1COC1] | (4-chlorophenyl)[6-(2-methyl-1-oxetan- 3-ylbenzimidazol-5-yl)-2-morpholin-4- ylpyrimidin-4-yl]amine |
| 319 | 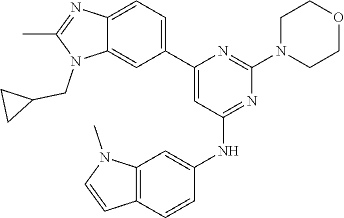 [SMILES: Cc1nc2ccc(-c3cc(Nc4ccc5ccn(C)c5c4)nc(N4CCOCC4)n3)cc2n1CC1CC1] | {6-[1-(cyclopropylmethyl)-2- methylbenzimidazol-6-yl]-2-morpholin- 4-ylpyrimidin-4-yl}(1-methylindol-6- yl)amine |
| 320 | 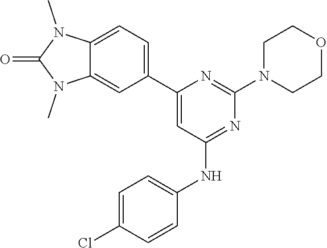 [SMILES: Cn1c(=O)n(C)c2cc(-c3cc(Nc4ccc(Cl)cc4)nc(N4CCOCC4)n3)ccc21] | 5-{6-[(4-chlorophenyl)amino]-2- morpholin-4-ylpyrimidin-4-yl}-1,3- dimethyl-3-hydrobenzimidazol-2-one |
| 321 | 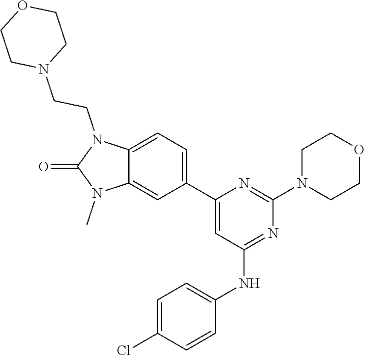 [SMILES: Cn1c(=O)n(CCN2CCOCC2)c2ccc(-c3cc(Nc4ccc(Cl)cc4)nc(N4CCOCC4)n3)cc21] | 5-{6-[(4-chlorophenyl)amino]-2- morpholin-4-ylpyrimidin-4-yl}-3- methyl-1-(2-morpholin-4-ylethyl)-3- hydrobenzimidazol-2-one |
| 322 | 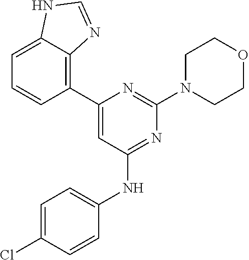 [SMILES: Clc1ccc(Nc2cc(-c3cccc4[nH]cnc34)nc(N3CCOCC3)n2)cc1] | (6-benzimidazol-4-yl-2-morpholin-4- ylpyrimidin-4-yl)(4-chlorophenyl)amine |
| 323 | 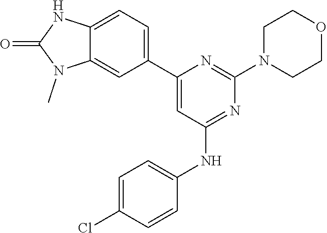 [SMILES: Cn1c(=O)[nH]c2ccc(-c3cc(Nc4ccc(Cl)cc4)nc(N4CCOCC4)n3)cc21] | 6-{6-[(4-chlorophenyl)amino]-2- morpholin-4-ylpyrimidin-4-yl}-1- methyl-3-hydrobenzimidazol-2-one |
| 324 | 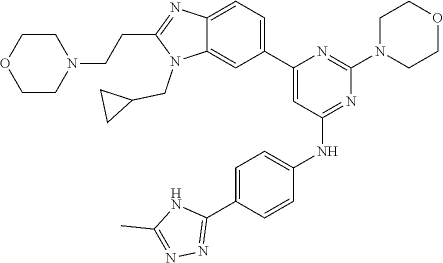 [SMILES: Cc1nnc(-c2ccc(Nc3cc(-c4ccc5nc(CCN6CCOCC6)n(CC6CC6)c5c4)nc(N4CCOCC4)n3)cc2)[nH]1] | 6-(1-(cyclopropylmethyl)-2-(2- morpholinoethyl)-1H-benzo[d]imidazol- 6-yl)-N-(4-(5-methyl-4H-1,2,4-triazol-3- yl)phenyl)-2-morpholinopyrimidin-4- amine |
| 325 | 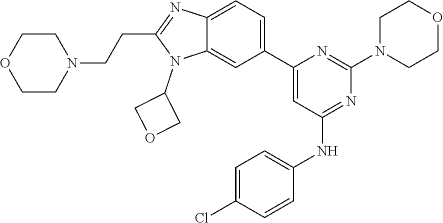 [SMILES: Clc1ccc(Nc2cc(-c3ccc4nc(CCN5CCOCC5)n(C5COC5)c4c3)nc(N3CCOCC3)n2)cc1] | N-(4-chlorophenyl)-2-morpholino-6-(2- (2-morpholinoethyl)-1-(oxetan-3-yl)- 1H-benzo[d]imidazol-6-yl)pyrimidin-4- amine |
| 326 | 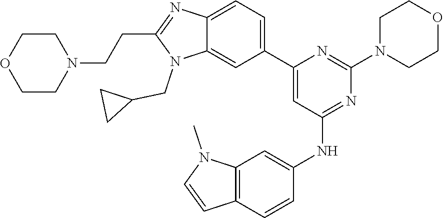 [SMILES: Cn1ccc2ccc(Nc3cc(-c4ccc5nc(CCN6CCOCC6)n(CC6CC6)c5c4)nc(N4CCOCC4)n3)cc21] | N-(6-(1-(cyclopropylmethyl)-2-(2- morpholinoethyl)-1H-benzo[d]imidazol- 6-yl)-2-morpholinopyrimidin-4-yl)-1- methyl-1H-indol-6-amine |
| 327 | 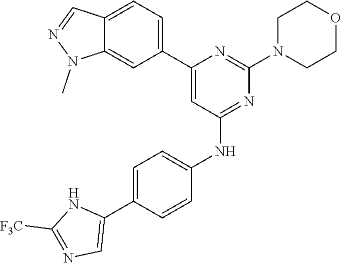 [SMILES: Cn1ncc2ccc(-c3cc(Nc4ccc(-c5cnc(C(F)(F)F)[nH]5)cc4)nc(N4CCOCC4)n3)cc21] | 6-(1-methyl-1H-indazol-6-yl)-2- morpholino-N-(4-(2-(trifluoromethyl)- 1H-imidazol-5-yl)phenyl)pyrimidin-4- amine |
| 328 | 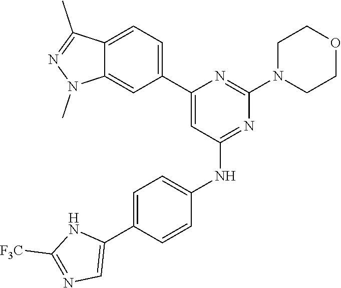 [SMILES: Cc1nn(C)c2cc(-c3cc(Nc4ccc(-c5cnc(C(F)(F)F)[nH]5)cc4)nc(N4CCOCC4)n3)ccc12] | 6-(1,3-dimethyl-1H-indazol-6-yl)-2- morpholino-N-(4-(2-(trifluoromethyl)- 1H-imidazol-5-yl)phenyl)pyrimidin-4- amine |
| 329 | 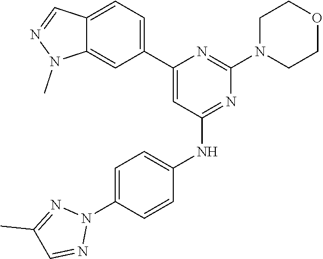 [SMILES: Cc1cnn(-c2ccc(Nc3cc(-c4ccc5cnn(C)c5c4)nc(N4CCOCC4)n3)cc2)n1] | 6-(1-methyl-1H-indazol-6-yl)-N-(4-(4- methyl-2H-1,2,3-triazol-2-yl)phenyl)-2- morpholinopyrimidin-4-amine |
| 330 | 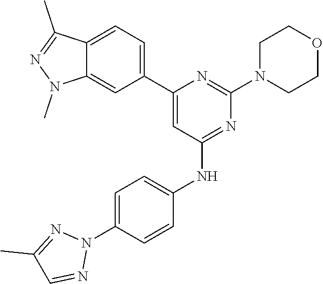 [SMILES: Cc1cnn(-c2ccc(Nc3cc(-c4ccc5c(C)nn(C)c5c4)nc(N4CCOCC4)n3)cc2)n1] | 6-(1,3-dimethyl-1H-indazol-6-yl)-N-(4- (4-methyl-2H-1,2,3-triazol-2- yl)phenyl)-2-morpholinopyrimidin-4- amine |
| 331 | 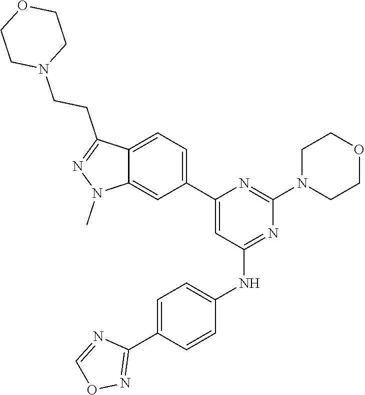 [SMILES: Cn1nc(CCN2CCOCC2)c2ccc(-c3cc(Nc4ccc(-c5ncon5)cc4)nc(N4CCOCC4)n3)cc21] | N-(4-(1,2,4-oxadiazol-3-yl)phenyl)-6- (1-methyl-3-(2-morpholinoethyl)-1H- indazol-6-yl)-2-morpholinopyrimidin-4- amine |
| 332 | 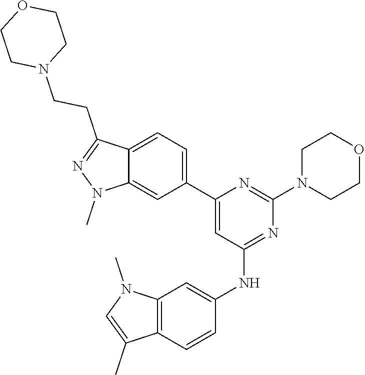 [SMILES: Cc1cn(C)c2cc(Nc3cc(-c4ccc5c(CCN6CCOCC6)nn(C)c5c4)nc(N4CCOCC4)n3)ccc12] | 1,3-dimethyl-N-(6-(1-methyl-3-(2- morpholinoethyl)-1H-indazol-6-yl)-2- morpholinopyrimidin-4-yl)-1H-indol-6- amine |
| 333 | 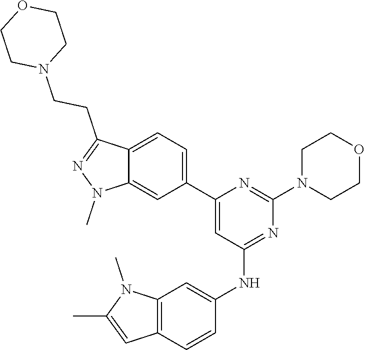 [SMILES: Cc1cc2ccc(Nc3cc(-c4ccc5c(CCN6CCOCC6)nn(C)c5c4)nc(N4CCOCC4)n3)cc2n1C] | 1,2-dimethyl-N-(6-(1-methyl-3-(2- morpholinoethyl)-1H-indazol-6-yl)-2- morpholinopyrimidin-4-yl)-1H-indol-6- amine |
| 334 | 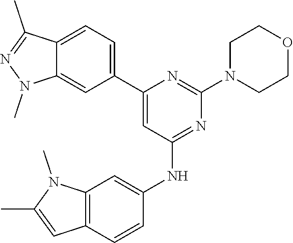 [SMILES: Cc1nn(C)c2cc(-c3cc(Nc4ccc5cc(C)n(C)c5c4)nc(N4CCOCC4)n3)ccc12] | N-(6-(1,3-dimethyl-1H-indazol-6-yl)-2- morpholinopyrimidin-4-yl)-1,2- dimethyl-1H-indol-6-amine |
| 335 | 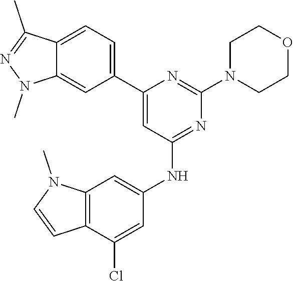 [SMILES: Cc1nn(C)c2cc(-c3cc(Nc4cc(Cl)c5ccn(C)c5c4)nc(N4CCOCC4)n3)ccc12] | 4-chloro-N-(6-(1,3-dimethyl-1H- indazol-6-yl)-2-morpholinopyrimidin-4- yl)-1-methyl-1H-indol-6-amine |
| 336 | 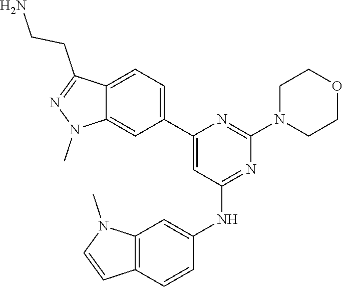 [SMILES: Cn1ccc2ccc(Nc3cc(-c4ccc5c(CCN)nn(C)c5c4)nc(N4CCOCC4)n3)cc21] | N-(6-(3-(2-aminoethyl)-1-methyl-1H- indazol-6-yl)-2-morpholinopyrimidin-4- yl)-1-methyl-1H-indol-6-amine |
| 337 | 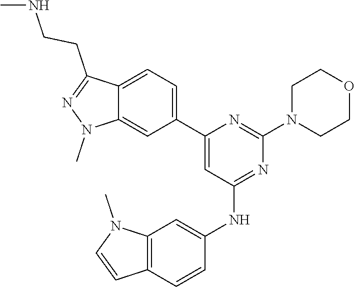 [SMILES: CNCCc1nn(C)c2cc(-c3cc(Nc4ccc5ccn(C)c5c4)nc(N4CCOCC4)n3)ccc12] | 1-methyl-N-(6-(1-methyl-3-(2- (methylamino)ethyl)-1H-indazol-6-yl)- 2-morpholinopyrimidin-4-yl)-1H-indol- 6-amine |
| 338 | 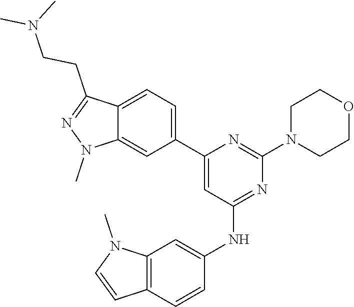 [SMILES: CN(C)CCc1nn(C)c2cc(-c3cc(Nc4ccc5ccn(C)c5c4)nc(N4CCOCC4)n3)ccc12] | N-(6-(3-(2-(dimethylamino)ethyl)-1- methyl-1H-indazol-6-yl)-2- morpholinopyrimidin-4-yl)-1-methyl- 1H-indol-6-amine |
| 339 | 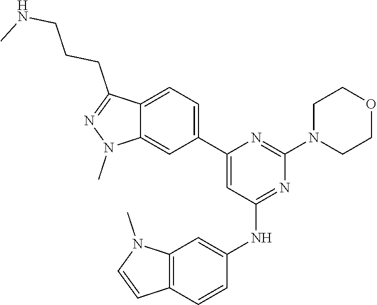 [SMILES: CNCCCc1nn(C)c2cc(-c3cc(Nc4ccc5ccn(C)c5c4)nc(N4CCOCC4)n3)ccc12] | 1-methyl-N-(6-(1-methyl-3-(3- (methylamino)propyl)-1H-indazol-6-yl)- 2-morpholinopyrimidin-4-yl)-1H-indol- 6-amine |
| 340 | 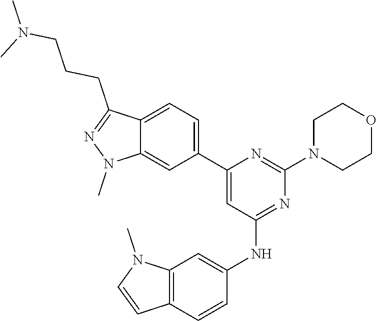 [SMILES: CN(C)CCCc1nn(C)c2cc(-c3cc(Nc4ccc5ccn(C)c5c4)nc(N4CCOCC4)n3)ccc12] | N-(6-(3-(3-(dimethylamino)propyl)-1- methyl-1H-indazol-6-yl)-2- morpholinopyrimidin-4-yl)-1-methyl- 1H-indol-6-amine |
| 341 | 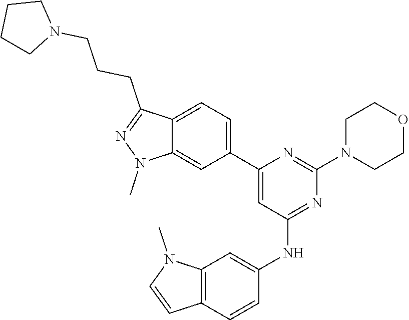 [SMILES: Cn1ccc2ccc(Nc3cc(-c4ccc5c(CCCN6CCCC6)nn(C)c5c4)nc(N4CCOCC4)n3)cc21] | 1-methyl-N-(6-(1-methyl-3-(3- (pyrrolidin-1-yl)propyl)-1H-indazol-6- yl)-2-morpholinopyrimidin-4-yl)-1H- indol-6-amine |
| 342 | 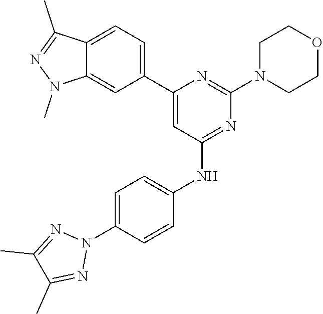 [SMILES: Cc1nn(-c2ccc(Nc3cc(-c4ccc5c(C)nn(C)c5c4)nc(N4CCOCC4)n3)cc2)nc1C] | 6-(1,3-dimethyl-1H-indazol-6-yl)-N-(4- (4,5-dimethyl-2H-1,2,3-triazol-2- yl)phenyl)-2-morpholinopyrimidin-4- amine |
| 343 | 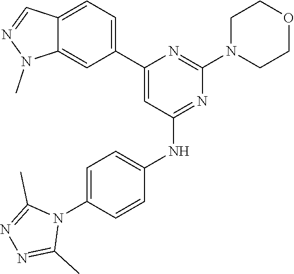 [SMILES: Cc1nnc(C)n1-c1ccc(Nc2cc(-c3ccc4cnn(C)c4c3)nc(N3CCOCC3)n2)cc1] | N-(4-(3,5-dimethyl-4H-1,2,4-triazol-4- yl)phenyl)-6-(1-methyl-1H-indazol-6- yl)-2-morpholinopyrimidin-4-amine |
| 344 | 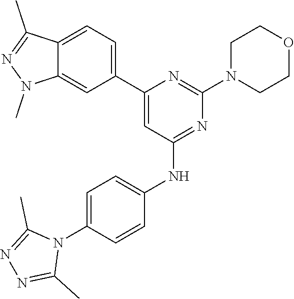 [SMILES: Cc1nn(C)c2cc(-c3cc(Nc4ccc(-n5c(C)nnc5C)cc4)nc(N4CCOCC4)n3)ccc12] | 6-(1,3-dimethyl-1H-indazol-6-yl)-N-(4- (3,5-dimethyl-4H-1,2,4-triazol-4- yl)phenyl)-2-morpholinopyrimidin-4- amine |
| 345 | 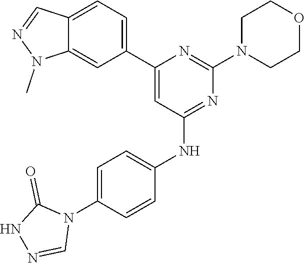 [SMILES: Cn1ncc2ccc(-c3cc(Nc4ccc(-n5cn[nH]c5=O)cc4)nc(N4CCOCC4)n3)cc21] | 4-(4-((6-(1-methyl-1H-indazol-6-yl)-2- morpholinopyrimidin-4- yl)amino)phenyl)-1H-1,2,4-triazol- 5(4H)-one |
| 346 | 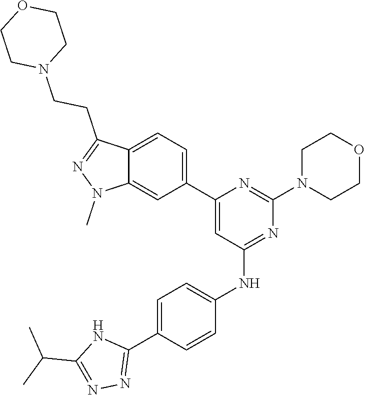 [SMILES: CC(C)c1nnc(-c2ccc(Nc3cc(-c4ccc5c(CCN6CCOCC6)nn(C)c5c4)nc(N4CCOCC4)n3)cc2)[nH]1] | N-(4-(5-isopropyl-4H-1,2,4-triazol-3- yl)phenyl)-6-(1-methyl-3-(2- morpholinoethyl)-1H-indazol-6-yl)-2- morpholinopyrimidin-4-amine |
| 347 | 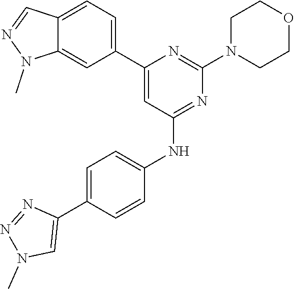 [SMILES: Cn1cc(-c2ccc(Nc3cc(-c4ccc5cnn(C)c5c4)nc(N4CCOCC4)n3)cc2)nn1] | N-(4-(1-methyl-1H-1,2,3-triazol-4- yl)phenyl)-6-(1-methyl-1H-indazol-6- yl)-2-morpholinopyrimidin-4-amine |
| 348 | 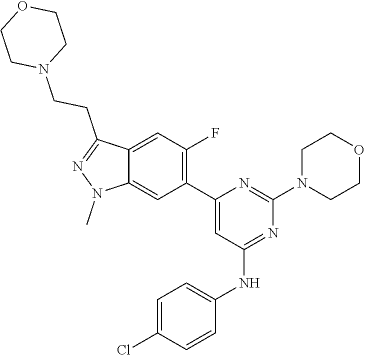 [SMILES: Cn1nc(CCN2CCOCC2)c2cc(F)c(-c3cc(Nc4ccc(Cl)cc4)nc(N4CCOCC4)n3)cc21] | N-(4-chlorophenyl)-6-(5-fluoro-1- methyl-3-(2-morpholinoethyl)-1H- indazol-6-yl)-2-morpholinopyrimidin-4- amine |
| 349 | 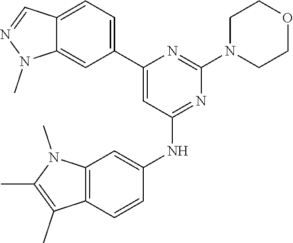 [SMILES: Cc1c(C)n(C)c2cc(Nc3cc(-c4ccc5cnn(C)c5c4)nc(N4CCOCC4)n3)ccc12] | 1,2,3-trimethyl-N-(6-(1-methyl-1H- indazol-6-yl)-2-morpholinopyrimidin-4- yl)-1H-indol-6-amine |
| 350 | 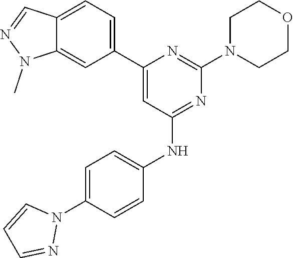 [SMILES: Cn1ncc2ccc(-c3cc(Nc4ccc(-n5cccn5)cc4)nc(N4CCOCC4)n3)cc21] | N-(4-(1H-pyrazol-1-yl)phenyl)-6-(1- methyl-1H-indazol-6-yl)-2- morpholinopyrimidin-4-amine |
| 351 | 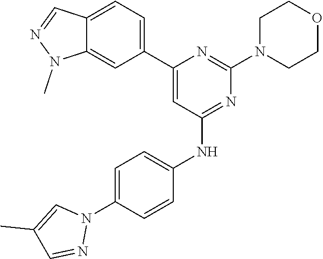 [SMILES: Cc1cnn(-c2ccc(Nc3cc(-c4ccc5cnn(C)c5c4)nc(N4CCOCC4)n3)cc2)c1] | 6-(1-methyl-1H-indazol-6-yl)-N-(4-(4- methyl-1H-pyrazol-1-yl)phenyl)-2- morpholinopyrimidin-4-amine |
| 352 | 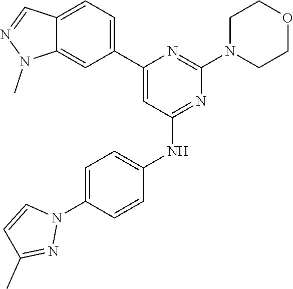 [SMILES: Cc1ccn(-c2ccc(Nc3cc(-c4ccc5cnn(C)c5c4)nc(N4CCOCC4)n3)cc2)n1] | 6-(1-methyl-1H-indazol-6-yl)-N-(4-(3- methyl-1H-pyrazol-1-yl)phenyl)-2- morpholinopyrimidin-4-amine |
| 353 | 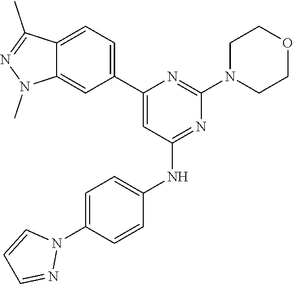 [SMILES: Cc1nn(C)c2cc(-c3cc(Nc4ccc(-n5cccn5)cc4)nc(N4CCOCC4)n3)ccc12] | N-(4-(1H-pyrazol-1-yl)phenyl)-6-(1,3- dimethyl-1H-indazol-6-yl)-2- morpholinopyrimidin-4-amine |
| 354 | 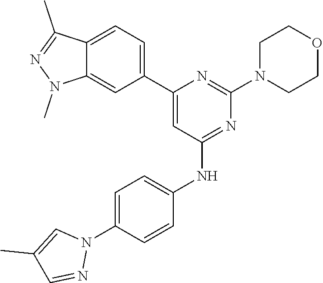 [SMILES: Cc1cnn(-c2ccc(Nc3cc(-c4ccc5c(C)nn(C)c5c4)nc(N4CCOCC4)n3)cc2)c1] | 6-(1,3-dimethyl-1H-indazol-6-yl)-N-(4- (4-methyl-1H-pyrazol-1-yl)phenyl)-2- morpholinopyrimidin-4-amine |
| 355 | 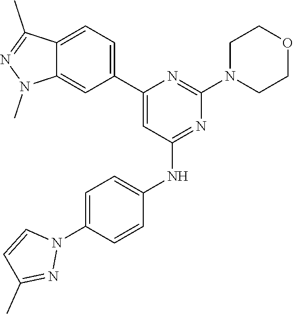 [SMILES: Cc1ccn(-c2ccc(Nc3cc(-c4ccc5c(C)nn(C)c5c4)nc(N4CCOCC4)n3)cc2)n1] | 6-(1,3-dimethyl-1H-indazol-6-yl)-N-(4- (3-methyl-1H-pyrazol-1-yl)phenyl)-2- morpholinopyrimidin-4-amine |
| 356 | 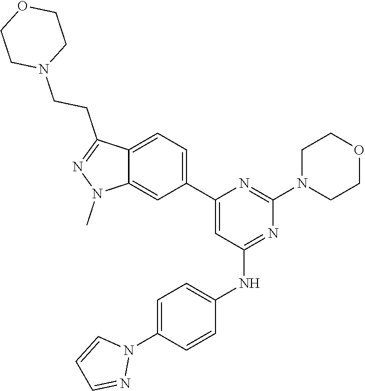 [SMILES: Cn1nc(CCN2CCOCC2)c2ccc(-c3cc(Nc4ccc(-n5cccn5)cc4)nc(N4CCOCC4)n3)cc21] | N-(4-(1H-pyrazol-1-yl)phenyl)-6-(1- methyl-3-(2-morpholinoethyl)-1H- indazol-6-yl)-2-morpholinopyrimidin-4- amine |
| 357 | 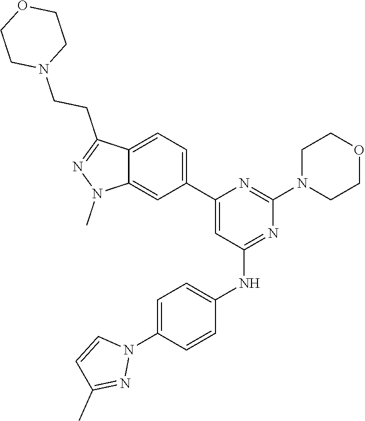 [SMILES: Cc1ccn(-c2ccc(Nc3cc(-c4ccc5c(CCN6CCOCC6)nn(C)c5c4)nc(N4CCOCC4)n3)cc2)n1] | N-(4-(3-methyl-1H-pyrazol-1- yl)phenyl)-6-(1-methyl-3-(2- morpholinoethyl)-1H-indazol-6-yl)-2- morpholinopyrimidin-4-amine |
| 358 | 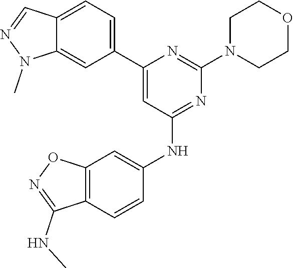 [SMILES: CNc1noc2cc(Nc3cc(-c4ccc5cnn(C)c5c4)nc(N4CCOCC4)n3)ccc12] | N3-methyl-N6-(6-(1-methyl-1H- indazol-6-yl)-2-morpholinopyrimidin-4- yl)benzo[d]isoxazole-3,6-diamine |
| 359 | 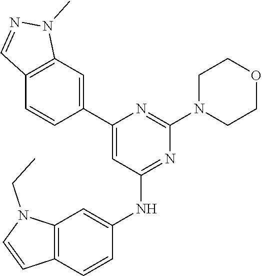 [SMILES: CCn1ccc2ccc(Nc3cc(-c4ccc5cnn(C)c5c4)nc(N4CCOCC4)n3)cc21] | (1-ethylindol-6-yl)[6-(1-methyl(1H- indazol-6-yl))-2-morpholin-4- ylpyrimidin-4-yl]amine |
| 360 | 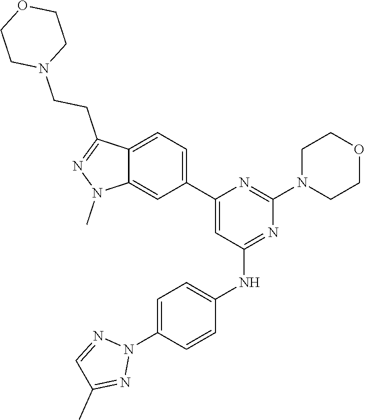 [SMILES: Cc1cnn(-c2ccc(Nc3cc(-c4ccc5c(CCN6CCOCC6)nn(C)c5c4)nc(N4CCOCC4)n3)cc2)n1] | N-(4-(4-methyl-2H-1,2,3-triazol-2- yl)phenyl)-6-(1-methyl-3-(2- morpholinoethyl)-1H-indazol-6-yl)-2- morpholinopyrimidin-4-amine |
| 361 | 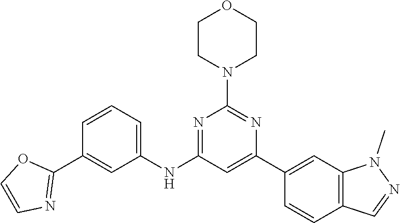 [SMILES: Cn1ncc2ccc(-c3cc(Nc4cccc(-c5ncco5)c4)nc(N4CCOCC4)n3)cc21] | 6-(1-methyl-1H-indazol-6-yl)-2- morpholino-N-(3-(oxazol-2- yl)phenyl)pyrimidin-4-amine |
| 362 | 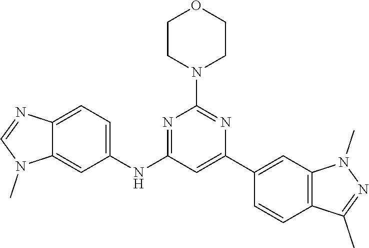 [SMILES: Cc1nn(C)c2cc(-c3cc(Nc4ccc5ncn(C)c5c4)nc(N4CCOCC4)n3)ccc12] | N-(6-(1,3-dimethyl-1H-indazol-6-yl)-2- morpholinopyrimidin-4-yl)-1-methyl- 1H-benzo[d]imidazol-6-amine |
| 363 | 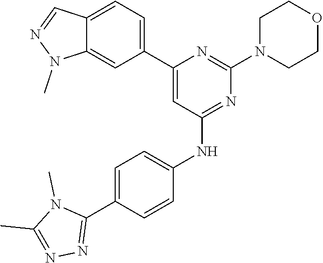 [SMILES: Cc1nnc(-c2ccc(Nc3cc(-c4ccc5cnn(C)c5c4)nc(N4CCOCC4)n3)cc2)n1C] | N-(4-(4,5-dimethyl-4H-1,2,4-triazol-3- yl)phenyl)-6-(1-methyl-1H-indazol-6- yl)-2-morpholinopyrimidin-4-amine |

In certain embodiments, provided are compound nos. 5-11, 15-16, 19-112, 115-126, 129-137, 147, 151-152, 157, 162-175, 179-196, 201, 203-206, 208-220, 226-234, 239-240, 242-318, 320-325, 327-331, 342-348, 350-357, 360-361, and 363. In certain embodiments, provided are compound nos. 1-4, 12-14, 17-18, 113-114, 127-128, 138-146, 148-150, 153-156, 158-161, 176-178, 197-200, 202, 207, 221-225, 235-238, 241, 319, 326, 332-341, 349, 358-359, and 362.

In certain embodiments, provided are compound nos. 1-210, 221, and 327-363. In certain embodiments, provided are compound nos. 222-326. In certain embodiments, provided are compound nos. 211-220.

In certain embodiments, provided are compound nos. 1, 4, 7, 11, 14, 16, 18, 20, and 48-52. In certain embodiments, provided are compound nos. 2-3, 5-6, 8-10, 12-13, 15, 17, 19, 21-47, 53-210, 221, and 327-363.

In certain embodiments, provided are compound nos. 13, 19, 40, 41, 42, 45, 47, 55, 81, 91, 92, 109, 117, 118, 122, 123, 124, 133, 134, 136, 149, 159, 163, 167, 168, 169, 173, 174, 176, 180, 181, 189, 190, 191, 196, 202, 207, 208, 209, 213, 214, 215, 217, 218, 226, 236, 239, 240, 247, 257, 258, 259, 260, 265, 266, 267, 277, 283, 287, 292, 302, 304, 306, 313, 316, 318, 319, 320, 323, 326, 329, 330, 336, 340, 350, 352, 353, 355, 356, 357, 360, and 372.

In certain embodiments, provided are compound nos. 2, 3, 4, 5, 12, 14, 20, 24, 28, 34, 36, 37, 43, 44, 46, 51, 53, 60, 61, 62, 64, 66, 67, 69, 70, 71, 73, 74, 75, 76, 78, 79, 82, 84, 85, 86, 96, 97, 98, 102, 110, 111, 112, 115, 119, 130, 131, 137, 139, 147, 157, 158, 161, 164, 165, 171, 172, 177, 178, 179, 184, 186, 187, 193, 194, 200, 201, 204, 205, 206, 211, 212, 221, 223, 224, 225, 227, 229, 230, 231, 232, 233, 237, 238, 241, 242, 243, 244, 245, 246, 248, 249, 252, 254, 255, 263, 269, 271, 274, 276, 279, 280, 282, 285, 286, 288, 295, 299, 305, 308, 310, 315, 317, 321, 322, 332, 333, 334, 341, and 348.

In certain embodiments, provided is a pharmaceutical composition comprising 1) a compound described herein, optionally as a single stereoisomer or mixture of stereoisomers thereof and additionally optionally as a pharmaceutically acceptable salt thereof, and 2) a pharmaceutically acceptable excipient or pharmaceutically acceptable carrier.

In certain embodiments, provided is a pharmaceutical composition comprising 1) a Compound of Formula I, I(a), I(b), I(c), I(d), I(e), I(f), I(g), I(h), I(i), I(j), or I(k), or a compound in Table 1 optionally as a tautomer, a single stereoisomer or mixture of stereoisomers thereof and additionally optionally as a pharmaceutically acceptable salt thereof, and 2) a pharmaceutically acceptable excipient or pharmaceutically acceptable carrier.

In certain embodiments, provided is a pharmaceutical composition comprising 1) a Compound of Formula I, I(a), I(b), I(c), I(d), I(e), I(f), I(g), I(h), I(i), I(j), or I(k), or Formula II, or a compound in Table 1, optionally as a single stereoisomer or mixture of stereoisomers thereof and additionally optionally as a pharmaceutically acceptable salt thereof, and 2) a pharmaceutically acceptable excipient or pharmaceutically acceptable carrier.

In certain embodiments, the compounds presented herein can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumor, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

In certain embodiments, the compounds presented herein can be administered in any acceptable solid, semi-solid, liquid or gaseous dosage form. Acceptable dosage forms include, but are not limited to, aerosols, capsules, creams, elixirs, emulsions, gases, gels, grains, liniments, lotions, lozenges, ointments, pastes, powders, solutions, suspensions, syrups and tablets. Acceptable delivery systems include, but are not limited to, biodegradable implants (e.g., poly(DL-lactide), lactide/glycolide copolymers and lactide/caprolactone copolymers), capsules, douches, enemas, inhalers, intrauterine devices, nebulizers, patches, pumps and suppositories. Methods for preparing the dosage forms of the invention are known, or will be apparent, to those skilled in this art; for example, see Remington's Pharmaceutical Sciences, 18th Ed., (Mack Publishing Company, Easton, Pa., 1990).

In certain embodiments, a dosage form of the invention may be comprised solely of a compound of the invention or the compound of the invention may be formulated along with conventional excipients, including pharmaceutical carriers, adjuvants, and/or other medicinal or pharmaceutical agents. Acceptable excipients include, but are not limited to, (a) antiadherents, such as croscarmellose sodium, crosprovidone, sodium starch glycolate, microcrystalline cellulose, starch and talc; (b) binders, such as acacia, cellulose, gelatin, hydroxypropyl cellulose, lactose, maltitol, polyethylene glycol, polyvinyl pyrrolidone, sorbitol, starch, sugar, sucrose and xylitol; (c) coatings, such as cellulose, shellac, zein and enteric agents; (d) disintegrants, such as cellulose, crosslinked polyvinyl pyrrolidone, sodium carboxymethyl cellulose, methylcellulose, microcrystalline cellulose, sodium starch glycolate, starch, and alginic acid; (e) diluents or filling agents, such as calcium or sodium carbonate, calcium or sodium phosphate, sugars (such as glucose, lactose, mannitol, sorbitol and sucrose), cellulose, croscarmellose sodium, and povidone; (f) flavoring agents; (g) coloring agents; (h) glidants, such as calcium stearate, colloidal silicon dioxide, glyceryl behenate, glyceryl monostearate, glyceryl palmitostearate, hydrogenated vegetable oil, magnesium stearate, magnesium trisilicate, mineral oil, polyethylene glycols, silicon dioxide, starch, stearate, stearic acid, talc, sodium stearyl fumarate, sodium benzoate and zinc; (i) lubricants, such as calcium stearate, hydrogenated vegetable oils, magnesium stearate, mineral oil, polyethylene glycol, sodium stearyl fumarate, stearin, stearic acid and talc; and (j) preservatives, such as chlorobutanol, citric acid, cysteine, methionine, methyl paraben, phenol, propyl paraben, retinyl palmitate, selenium, sodium citrate, sorbic acid, vitamin A, vitamin C and vitamin E. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed. Capsules may contain any of the excipients listed above, and may additionally contain a semi-solid or liquid carrier, such as a polyethylene glycol or oil. Pharmaceutical carriers include soluble polymers, microparticles made of insoluble or biodegradable natural and synthetic polymers, microcapsules or microspheres, lipoproteins, liposomes and micelles.

In certain embodiments, the pharmaceutical compositions may be in the form of a liquid, such as a solution, suspension, emulsion, syrup, elixir, or other like forms or may be presented as a dry product for reconstitution with water or other suitable vehicle before use. Liquid preparations may contain conventional additives such as (a) liquid diluents, such as water, saline, Ringer's solution, alcohols including monohydric alcohols and polyhydric alcohols such as polyethylene or propylene glycols and their derivatives, glycerin, fixed oils such as synthetic mono or diglycerides, or other solvents; (b) surfactants, suspending agents, or emulsifying agents, such as sodium carboxymethylcellulose, methylcellulose, hydroxypropyl methylcelluose, sodium alginate, polyvinylpyrrolidone, gum tragacanth, gum acacia, polyoxyethylene sorbitan fatty acid esters, saturated polyglycolized glycerides, monoglycerides, fatty acid esters, block copolymers of ethylene oxide and propylene oxide, polyoxyl stearates, ethoxylated castor oils, and ethoxylated hydroxystearic acids; (c) buffers, such as acetates, citrates or phosphates; (d) chelating agents, such as ethylenediaminetetraacetic acid, carbohydrates such as dextran, hydroxyalkylcellulose, hydroxyalkylmethylcellulose, or saturated fatty acids, such as stearic acid; (e) antibacterial agents, such as chlorobutanol, benzyl alcohol, phenol, sorbic acid, or parabens, such as methyl paraben; (f) antioxidants, such as ascorbic acid or sodium bisulfite; (g) isotonic agents, sodium chloride or sugars, such as dextrose; as well as sweetening and flavoring agents, dyes and preservatives.

In certain embodiments, the pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In certain embodiments, the pharmaceutical compositions will contain a therapeutically effective amount of a compound of the invention, as an individual stereoisomer or mixture of stereoisomers, or a pharmaceutically acceptable salt thereof, with the remainder of the pharmaceutical composition comprised of one or more pharmaceutically acceptable excipients. Generally, for oral administration, a compound of the invention, as an individual stereoisomer or mixture of stereoisomers, or a pharmaceutically acceptable salt thereof will comprise from 1% to 99% by weight of a pharmaceutically acceptable composition, with the remainder of the composition comprised of one or more pharmaceutically acceptable excipients. Typically, a compound of the invention, as an individual stereoisomer or mixture of stereoisomers, or a pharmaceutically acceptable salt thereof will comprise from 5% to 75% by weight of a pharmaceutically acceptable composition, with the remainder of the composition comprised of one or more pharmaceutically acceptable excipients. For parenteral administration, a compound of the invention, as an individual stereoisomer or mixture of stereoisomers, or a pharmaceutically acceptable salt thereof will comprise from 0.01% to 1% by weight of a pharmaceutically acceptable composition.

In certain embodiments, a therapeutically effective amount of a compound of the invention will vary depending upon a sundry of factors including the activity, metabolic stability, rate of excretion and duration of action of the compound, the age, weight, general health, sex, diet and species of the subject, the mode and time of administration of the compound, the presence of adjuvants or additional therapeutically active ingredients in a composition, and the severity of the disease for which the therapeutic effect is sought.

In certain embodiments, the compounds presented herein can be administered to human subjects at dosage levels in the range of about 0.1 to about 10,000 mg per day. A normal human adult having a body weight of about 70 kilograms can be administered a dosage in the range of from about 0.15 μg to about 150 mg per kilogram of body weight per day. Typically, a normal adult human will be administered from about 0.1 mg to about 25 mg, or 0.5 mg to about 10 mg per kilogram of body weight per day. The compounds of the invention may be administered in one or more unit dose forms. The unit doses may be administered one to four times a day, or two times a day, or once a day. In an alternate method of describing an effective dose, an oral unit dose is one that is necessary to achieve a blood serum level of about 0.05 to 20 μg/ml or about 1 to 20 μg/ml in a subject. The optimum dose of a compound of the invention for a particular subject can be determined by one of ordinary skill in the art.

In certain embodiments, the compounds described herein are used in the preparation or manufacture of medicaments for the treatment of diseases or conditions in which inhibition of heparan sulfate biosynthesis ameliorates the disease or condition. In some embodiments, a method for treating any of the diseases or conditions described herein in a subject in need of such treatment, involves administration of pharmaceutical compositions containing at least one compound described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In certain embodiments, provided is a method of treating or ameliorating a medical condition, comprising administering to a subject in need thereof a compound according to any of the various embodiments described herein or a pharmaceutical composition according to any of the various embodiments described herein.

In certain embodiments, provided herein is a method of treating or ameliorating a disease by the inhibition of heparan sulfate biosynthesis comprising administering to a subject in need of treatment a therapeutically-effective amount of a compound of Formula I, I(a), I(b), I(c), I(d), I(e), I(f), I(g), I(h), I(i), I(j), or I(k), or a compound in Table 1 optionally as a tautomer, a single stereoisomer or mixture of stereoisomers thereof and additionally optionally as a pharmaceutically acceptable salt thereof. In certain embodiments, the disease is selected from amyloid diseases (such as Alzheimer's disease, Parkinson's disease, type 2 diabetes, and chronic hemodialysis-related amyloid), multiple sclerosis, and an MPS disorder (such as MPS I, II, IIIA, IIIB, IIIC, IIID, and VII). In some embodiments, the diseases associated with abnormal HS accumulation are autoimmune disorders (such as multiple sclerosis, rheumatoid arthritis, juvenile chronic arthritis, Ankylosing spondylitis, psoriasis, psoriatic arthritis, adult still disease, Becet syndrome, familial Mediterranean fever, Crohn's disease, leprosy, osteomyelitis, tuberculosis, chronic bronchiectasis, Castleman disease), CNS disorders (such as Alzheimer's disease, Parkinson's disease, Huntington's disease, spongiform encephalopathies (Creutzfeld-Jakob, Kuru, Mad Cow)), chronic hemodialysis-related amyloidosis, diabetic amyloidosis, type-2 diabetes, and MPS I, II, IIIA, IIIB, IIIC, IIID, and VII disorders.

In certain embodiments, provided herein is a method of treating or ameliorating a disease by the inhibition of heparan sulfate biosynthesis comprising administering to a subject in need of treatment a therapeutically-effective amount of a compound of Formula I, I(a), I(b), I(c), I(d), I(e), I(f), I(g), I(h), I(i), I(j), or I(k), or a compound in Table 1 optionally as a tautomer, a single stereoisomer or mixture of stereoisomers thereof and additionally optionally as a pharmaceutically acceptable salt thereof in combination with enzyme replacement therapy. In certain embodiments, enzyme replacement therapy comprises administering to a patient in need thereof an enzyme which is missing or deficient in said patient. In certain embodiments, the combination therapy can be used to treat a lysosomal storage disorder (e.g. MPS).

In certain embodiments, the methods described herein can be conducted in living bodies of mammals, and in another embodiment, humans. In such a case, the compounds may be administered to the mammals, and in another embodiment, to the humans.

In certain embodiments, provided are articles of manufacture, comprising packaging material, a compound provided herein that is effective for modulating heparan sulfate biosynthesis, or for treatment, prevention or amelioration of one or more symptoms of a disease or condition in need of modulation of heparan sulfate biosynthesis, within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for modulating heparan sulfate biosynthesis, or for treatment, prevention or amelioration of one or more symptoms of disease or condition in need of modulation of heparan sulfate biosynthesis, are provided.

In certain embodiments, provided are kits comprising a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the containers are formed from a variety of materials such as glass or plastic.

In certain embodiments, the articles of manufacture and kits provided herein contain packaging materials. Packaging materials for use in packaging pharmaceutical products include, but are not limited to, blister packs, bottles, tubes, inhalers, pumps, bags, vials, containers, syringes, bottles, and any packaging material suitable for a selected formulation and intended mode of administration and treatment.

### Preparation of Compounds

The following are illustrative examples of how the compounds can be prepared and tested. Although the examples can represent only some embodiments, it should be understood that the following examples are illustrative and not limiting.

In a further aspect, it is provided a method of making a compound, comprising synthesizing a compound as any of the various embodiments described above or below. Examples of the method are further described in the Examples.

Compounds disclosed herein are commercially available or can be readily prepared from commercially available starting materials according to established methodology in the art of organic synthesis. General methods of synthesizing the compound can be found in, e.g., Stuart Warren and Paul Wyatt, Workbook for Organic Synthesis: The Disconnection Approach, second Edition, Wiley, 2010. Synthesis of some of the compounds are exemplified in detail below.

In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral axillary, separation of the resulting mixture of diastereomers by recrystallization or chromatography and liberation of the optically pure product from the auxiliary or (2) direct separation of the mixture of optical enantiomers on chiral chromatographic column

Materials were obtained from commercial suppliers and were used without further purification. Air or moisture sensitive reactions were conducted under argon atmosphere using oven-dried glassware and standard syringe/septa techniques. ¹H NMR spectra were measured at 400 MHz unless stated otherwise and data were reported as follows in ppm (δ) from the internal standard (TMS, 0.0 ppm): chemical shift (multiplicity, integration, coupling constant in Hz).

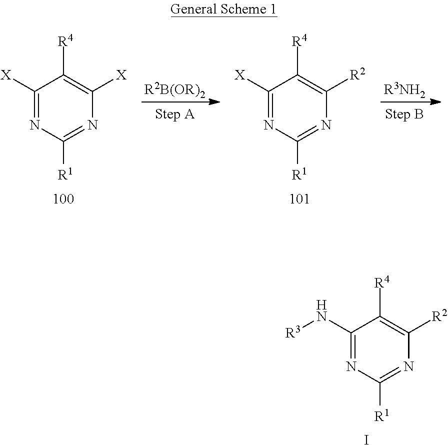

A Compound of Formula I (where each X is halo and all groups are as defined in the Summary of the Invention for a compound of Formula I or according to any of the embodiments disclosed herein) can be prepared according to General Scheme 1. In another embodiment each X is chloro.

Step A: An intermediate of formula 100 can be prepared using procedures known to one of skill in the art or is commercially available. An intermediate of formula 101 can be prepared using standard Suzuki coupling conditions, including microwave irradiation. In one example, the intermediate of formula 100 is treated in one embodiment at elevated temperature (for example 50-120° C.) with a boronic acid or ester of formula R²B(OR)₂ (where each R is hydrogen or alkyl or together with the atoms to which they are attached form a carbocyclic ring) in the presence of a base such as KF, K₃PO₄, Cs₂CO₃, K₂CO₃, Na₂CO₃, NaOtBu, KOtBu, NaOMe, NaOEt, Ba(OH)₂, or CsF, in the presence of a catalyst such as Pd(OAc)₂, Pd₂(dba)₃, PdCl₂(dppf), PdCl₂(P(cy)₃)₂, or Pd(PPh₃)₄, and in one or more solvents such as DMF, DMA, DCM, toluene, NMP, EtOH/DME/H₂O, THF, DME, or 1,4-dioxane.

Alternatively, Step A can performed using standard Stille coupling conditions. The intermediate of formula 100 is treated in one embodiment at elevated temperature (for example 50-120° C.) with an intermediate of formula R²Sn(alkyl)₃ in the presence of a catalyst such as a Pd(0) catalyst such as Pd(PPh₃)₄, PdCl₂(PPh₃)₂, or Pd₂(dba)₃ optionally in the presence of CuI, or LiCl, in the presence of a base such as CsF, Cs₂CO₃, and K₂CO₃, in one or more solvents such as NMP, toluene, and DMF.

Step B: The Compound of Formula I can then be prepared using standard Buchwald chemistry. The intermediate of formula 101 is treated in one embodiment at elevated temperature (for example 50-120° C.) with an amine of formula R³NH₂ in the presence of a base such as Cs₂CO₃, NaOtBu, KOtBuO, K₃PO₄, or K₂CO₃, in the presence of a catalyst such as Pd(OAc)₂, Pd₂(dba)₃, PdCl₂(dppf), CuI, or Pd(PPh₃)₄, and optionally in the presence of a ligand or precatalyst such as BINAP, xantphos, Brettphos, Xphos, Sphos, L-proline, in one or more solvents such as DMF, DMA, 1,4-dioxane, toluene, and DCM. The mixture can optionally be purified using procedures known to one of ordinary skill in the art.

Alternatively, Step B can performed using standard Buchwald conditions. The intermediate 100 is treated in one embodiment at elevated temperature (for example 50-120° C.) with R³NH₂ in the presence of concentrated HCl in one or more solvents such as isopropanol. The mixture can optionally be purified using procedures known to one of ordinary skill in the art.

Alternatively, Step B can performed using standard Ullmann coupling conditions. The intermediate 100 is treated in one embodiment at elevated temperature (for example 50-120° C.) with R³NH₂ in the presence of one or more catalysts such as Cu, CuI, and CuO, optionally in the presence of a base such as K₂CO₃, and K₃PO₄, in one or more solvent such as DMF, 2-ethoxyethanol, xylene, DMSO, and isopropanol. The mixture can optionally be purified using procedures known to one of ordinary skill in the art.

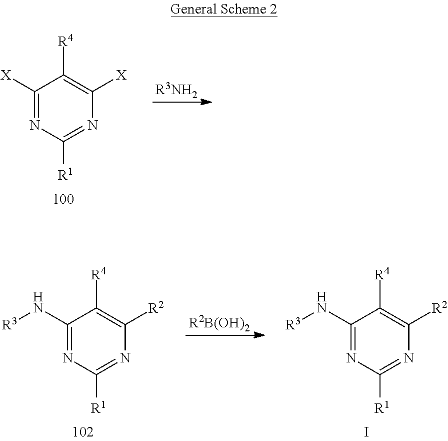

Alternatively, a Compound of Formula I (where X is halo and all groups are as defined in the Summary of the Invention for a compound of Formula I or according to any of the embodiments disclosed herein) can be prepared according to General Scheme 2 where intermediate 102 is prepared using conditions as described above for Step B in General Scheme 1 followed by conditions as described above for Step A in General Scheme 1. In another embodiment each X is chloro.

### SYNTHETIC EXAMPLES

The following describes ways in which the compounds described herein were or can be prepared. A person of ordinary skill in the art would know that variations in the synthetic procedures could be used to make the compounds.

### General Procedure for Boronate Ester Preparation

To a mixture of an intermediate of formula R²Br (1 eq), potassium acetate (3 eq) and bis(pinacolato)diboron (1.1 eq) in 1,4-dioxane, argon was bubbled through the solution for 15 min. 1,1′-Bis(diphenylphosphino) ferrocene palladium(II)dichloride dichloromethane adduct (PdCl₂(dppf).CH₂Cl₂) (0.1 eq) was added and the reaction mixture was stirred at 90° C. for overnight. The progress of the reaction was monitored by TLC. After completion of the reaction, the reaction mixture was filtered through celite, evaporated to dryness to afford the desired boronate ester,

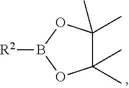

as crude product and used as such for the next step without further purification.

### General Procedure for Suzuki Coupling

To a mixture of an intermediate of formula 100 (1 eq) or formula 102 (1 eq), a boronic acid of formula R²B(OH)₂ or boronate ester of formula

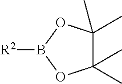

(1 eq) in 1,4-dioxane, 2 M solution of potassium phosphate was added and purged with argon for 15 min followed by the addition of tetrakis triphenyl phosphine palladium (0.06 eq) and stirred at 90° C. for overnight. The progress of the reaction was monitored by TLC. After completion of the reaction, the reaction mixture was filtered through celite and evaporated to dryness. The residue was taken in ethyl acetate, washed with water, brine, dried over anhydrous sodium sulfate and evaporated under reduced pressure. The crude product was purified by column chromatography/preparative HPLC to afford the intermediate of formula 101 or a Compound of Formula I, respectively.

### General Procedure for Buchwald Coupling

A mixture of the intermediate of formula 101 (1 eq) or formula 100, R³NH₂ (1 eq) and cesium carbonate (1.5 eq) in 1,4-dioxane was taken and purged with argon for 10 min, followed by the addition of BINAP (0.22 eq) and purged argon for additional 5 min. Palladium acetate (0.2 eq) was added and stirred at 100° C. for overnight. The progress of the reaction was monitored by TLC. After completion of the reaction, the reaction mixture was filtered through celite and evaporated to dryness. The residue was taken in ethyl acetate, washed with water, brine, dried over anhydrous sodium sulfate and evaporated under reduced pressure. The crude product was purified by column chromatography on silica gel to afford a Compound of Formula I or formula 102, respectively.

### General Procedure for Ester Hydrolysis

To a stirred solution of ester (1 eq) in methanol:water (1:1), NaOH (2 eq) was added and refluxed for 2-4 h. The progress of the reaction was monitored by TLC. After completion of the reaction, methanol was evaporated to dryness. Aqueous layer was washed with ethyl acetate. Aqueous layer was acidified using 1N HCl and dried to afford the desired product and used as such for the next step.

To a stirred solution of ester compound (1 eq) in THF:H₂O (1:1), lithium hydroxide (2 eq) in minimum amount of water was added and stirred at room temperature for 2 h. The progress of the reaction was monitored by TLC. After completion of the reaction, the reaction mixture was extracted with ethyl acetate. The aqueous layer was acidified with 1N HCl and the solid obtained was filtered and dried in vacuo to afford the acid. The crude product has been used as such for the next step without further purification.

### General Procedure for Amide Coupling:

To a mixture of corresponding Acid (1 eq) and PYBOP/HATU (1.5 eq) in DMF, DIPEA (3 eq) was added and stirred at room temperature for 10 min. Methyl amine hydrochloride (1.2 eq) was added slowly and stirred at room temperature for 2 h. The progress of the reaction was monitored by TLC. After completion of the reaction, water was added and extracted with ethyl acetate. The combined organic extracts were dried over anhydrous sodium sulfate and evaporated under reduced pressure. The crude product was purified by column chromatography/preparative HPLC to afford the desired product.

### Example 1

### Synthesis of N-(4-chlorophenyl)-6-(1-methyl-1H-indazol-6-yl)-2-morpholinopyrimidin-4-amine

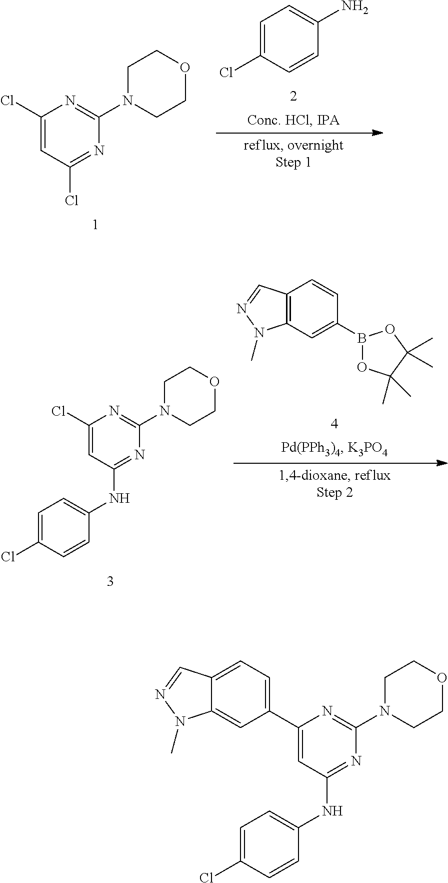

### Step 1: Synthesis of 6-chloro-N-(4-chlorophenyl)-2-morpholinopyrimidin-4-amine (3)

To a stirred solution of 4-(4,6-dichloropyrimidin-2-yl)morpholine 1 (1 g, 1 eq) and 4-chloro aniline 2 (0.547 g, 1 eq) in isopropanol (10 mL), concentrated HCl (2 mL) was added and heated to reflux at 100° C. for overnight. The progress of the reaction was monitored by TLC. After completion of the reaction, the reaction mixture was evaporated to dryness. The residue was taken in ethyl acetate (50 mL), washed with 1 N HCl, brine, dried over anhydrous sodium sulfate and evaporated under reduced pressure. The crude product was purified by column chromatography on silica gel 100-200 mesh using 20% EtOAc-hexane to afford the title compound 3. LCMS (m/z): 325.20 (M+1).

### Step 2: Synthesis of N-(4-chlorophenyl)-6-(1-methyl-1H-indazol-6-yl)-2-morpholinopyrimidin-4-amine

The title compound has been synthesized by following the General Procedure for Suzuki Coupling using compound 3 and Boronate ester 4 in Scheme 3. ¹H NMR (400 MHz, DMSO-d₆) δ: 10.06 (s, 1H), 8.22 (s, 1H), 8.13 (s, 1H), 7.89 (d, J=8.4 Hz, 1H), 7.75-7.65 (m, 3H), 7.41 (d, J=8.4 Hz, 2H), 6.73 (s, 1H), 4.14 (s, 3H), 3.84-3.72 (m, 8H); HPLC purity: 99.45%; LCMS Calculated for C₂₂H₂₁ClN₆O (free base): 420.15; Observed: 421.20 (M+1).

### Example 2

### Synthesis of N-(4-chlorophenyl)-6-(1-methyl-3-(2-morpholinoethyl)-1H-indazol-6-yl)-2-morpholino pyrimidin-4-amine

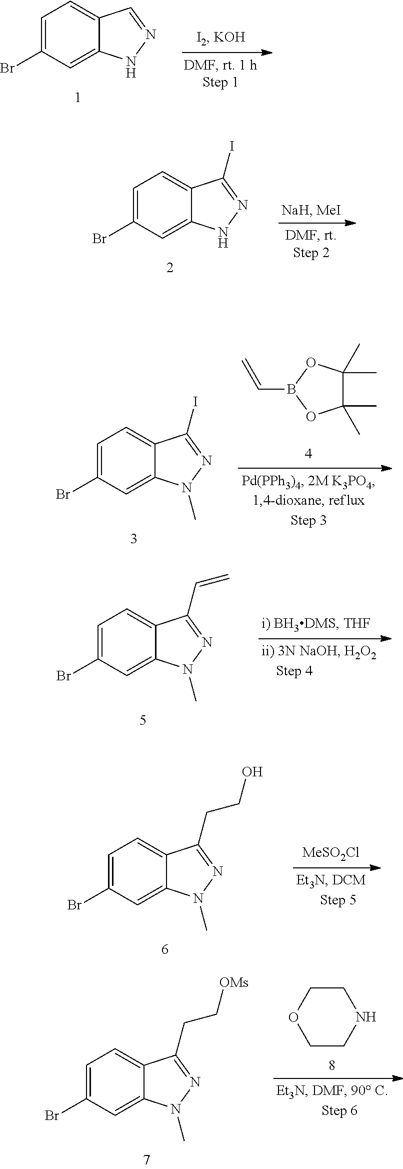

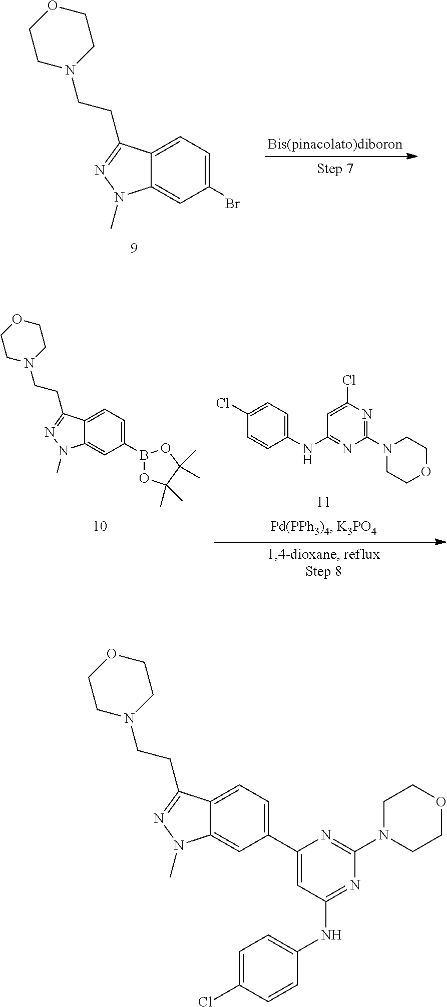 [SMILES: Cn1nc(CCN2CCOCC2)c2ccc(Br)cc21 ; Cn1nc(CCN2CCOCC2)c2ccc(B3OC(C)(C)C(C)(C)O3)cc21 ; Clc1ccc(Nc2cc(Cl)nc(N3CCOCC3)n2)cc1 ; Cn1nc(CCN2CCOCC2)c2ccc(-c3cc(Nc4ccc(Cl)cc4)nc(N4CCOCC4)n3)cc21]

### Step 1: Synthesis of 6-bromo-3-iodo-1H-indazole (2)

To a stirred solution of 6-bromo-1H-indazole 1 (60 g, 1 eq) and 3 N NaOH (600 mL) in 1,4-dioxane (1200 mL), Iodine (171 g, 2.2 eq) was added and stirred at room temperature for 1 h. The progress of the reaction was monitored by TLC. After completion of the reaction, the reaction mixture was quenched with 20% citric acid solution, saturated sodium bicarbonate solution and extracted with ethyl acetate (3×300 mL). Combined organic extracts were washed with brine, dried over anhydrous sodium sulfate and evaporated under reduced pressure to afford the crude compound 2. LCMS (m/z): 323.05 (M+1).

### Step 2: Synthesis of 6-bromo-3-iodo-1-methyl-1H-indazole (3)

To a stirred solution of 6-bromo-3-iodo-1H-indazole 2 (95 g, 1 eq) in DMF (150 mL), NaH (10.62 g, 1.5 eq) was added and stirred at room temperature for 10 min followed by the addition of methyl iodide (83.8 g, 2 eq). The reaction mixture was stirred for 30 min at room temperature. The progress of the reaction was monitored by TLC. After completion of the reaction, the reaction mixture was diluted with water and extracted with ethyl acetate (4×75 mL). Combined organic extracts were washed with brine, dried over anhydrous sodium sulfate and evaporated under reduced pressure. The crude product was purified by column chromatography on silica gel 100-120 mesh using 60% EtOAc-hexane to afford the title compound 3. LCMS (m/z): 336.95 (M+1).

### Step 3: Synthesis of 6-bromo-1-methyl-3-vinyl-1H-indazole (5)

The title compound has been synthesized by following the General Procedure for Suzuki Coupling using compound 3 and Boronate ester 4 in Scheme 4. LCMS (m/z): 239.05 (M+2).

### Step 4: Synthesis of 2-(6-bromo-1-methyl-1H-indazol-3-yl)ethanol (6)

To a stirred solution of compound 5 (25.6 g, 1 eq) in dry THF (400 mL), BH₃:DMS (432 mL, 8 eq) was added at 0° C. and stirred at room temperature for 16 h. The progress of the reaction was monitored by TLC. After completion of the reaction, the reaction mixture was quenched with 3N NaOH and 30% H₂O₂ solution at 0° C. The reaction mixture was stirred at room temperature for 3 h and extracted with ethyl acetate (4×75 mL). Combined organic extracts were dried over anhydrous sodium sulfate and evaporated under reduced pressure. The crude product was purified by column chromatography on silica gel 100-120 mesh using 60% EtOAc-hexane to afford the title compound 6. LCMS (m/z): 255.05 (M+1).

### Step 5: Synthesis of 2-(6-bromo-1-methyl-1H-indazol-3-yl)ethyl methanesulfonate(7)

To a stirred solution of compound 6 (12 g, 1 eq) in DCM (150 mL), triethylamine (9.54 g, 2 eq) was added and stirred for 15 min followed by the slow addition of mesyl chloride (8.07 g, 1.5 eq) at 0° C. The reaction mixture was stirred at room temperature for 1 h. The progress of the reaction was monitored by TLC. After completion of the reaction, the reaction mixture was diluted with water, extracted with dichloromethane (3×50 mL). The combined organic extracts were washed with brine, dried over anhydrous sodium sulfate and evaporated under reduced pressure to afford compound 7. LCMS (m/z): 333.05 (M+1).

### Step 6: Synthesis of 4-(2-(6-bromo-1-methyl-1H-indazol-3-yl)ethyl)morpholine (9)

To a stirred solution of compound 8 (5.81 g, 1.5 eq) in DMF (50 mL), triethylamine (9 g, 2.0 eq) was added and stirred at room temperature for 15 min. Compound 7 (14.8 g, 1 eq) in DMF (100 mL) was added and the reaction mixture was stirred at 90° C. for 1 h. The progress of the reaction was monitored by TLC. After completion of the reaction, the reaction mixture was quenched with water and extracted with ethyl acetate (3×100 mL). The combined organic extracts were washed with brine, dried over anhydrous sodium sulfate and evaporated under reduced pressure. The crude product was purified by column chromatography on silica gel 100-200 mesh using 3% MeOH-DCM to afford the title compound 9. LCMS (m/z): 325.15 (M+1).

### Step 7: Synthesis of 4-(2-(1-methyl-6-(4,4,5,5-tetramethyl-1,3,2-dioxaborolan-2-yl)-1H-indazol-3-yl) ethyl)morpholine (10)

The title compound has been synthesized by following the General Procedure for Boronate Ester Preparation described above using bromo compound 9 in Scheme 4 and Bis (pinacolato) diboron. LCMS (m/z): 372.45 (M+1).

### Step 8: Synthesis of N-(4-chlorophenyl)-6-(1-methyl-3-(2-morpholinoethyl)-1H-indazol-6-yl)-2-morpholino pyrimidin-4-amine

The title compound has been synthesized by following the General Procedure for Suzuki Coupling described above using compound 11 and Boronate ester 10 in Scheme 4 to afford the title compound. ¹H NMR (400 MHz, MeOD) δ: 8.11 (d, J=1.4 Hz, 1H), 8.04 (d, J=8.5 Hz, 1H), 7.65 (d, J=8.3 Hz, 2H), 7.55 (dd, J=8.5, 1.5 Hz, 1H), 7.50-7.41 (m, 2H), 6.61 (s, 1H), 4.16 (s, 3H), 4.13-4.10 (m, 2H), 3.94-3.78 (m, 10H), 3.76-3.64 (m, 4H), 3.56 (dd, J=8.5, 6.8 Hz, 2H), 3.38-3.24 (m, 2H); HPLC purity: 99.62%; LCMS Calculated for C₂₈H₃₂ClN₇O₂ (free base): 533.23; observed: 534.40 (M+1).

### Example 3

### Synthesis of N-(4-(2H-1,2,3-triazol-2-yl)phenyl)-6-(1-methyl-3-(2-morpholinoethyl)-1H-indazol-6-yl)-2-morpholinopyrimidin-4-amine

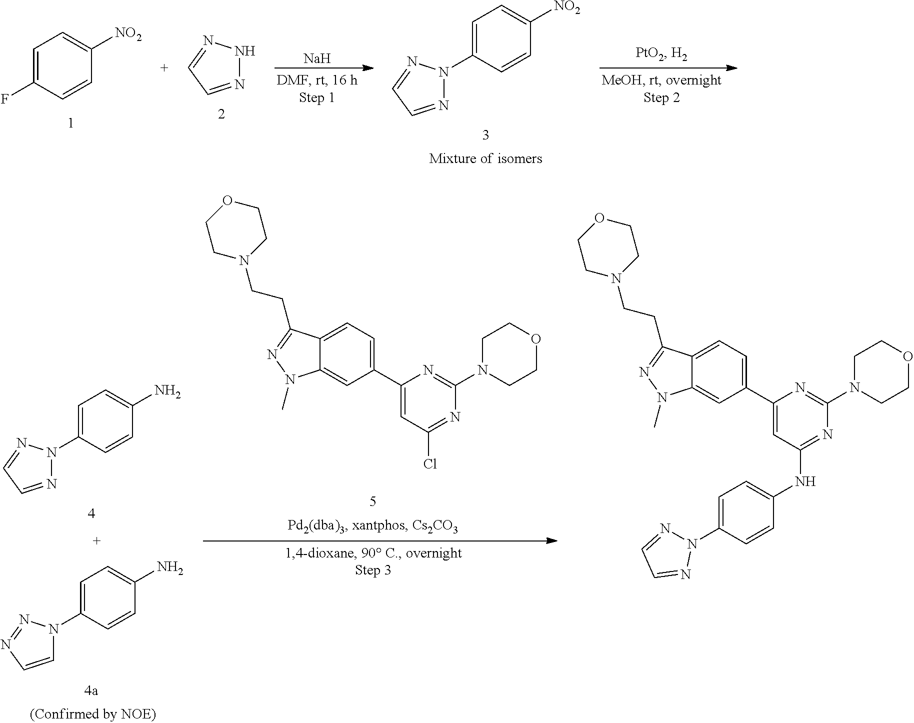

### Step 1: Synthesis of 2-(4-nitrophenyl)-2H-1,2,3-triazole (3)

To a stirred solution of NaH (0.58 g, 1 eq) in dry DMF (10 mL), compound 2 (1 g, 1 eq) was added at 0° C. and stirred for 15 min followed by the addition of compound 1 (2 g, 1 eq). The reaction mixture was stirred at room temperature for 16 h. The progress of the reaction was monitored by TLC. After completion of the reaction, the reaction mixture was quenched with ice cold water. The precipitated solid was collected by filtration, washed with water and dried under reduced pressure to afford the title compound 3 as mixture of isomers. LCMS (m/z): 190.95 (M+1).

### Step 2: Synthesis of 4-(2H-1,2,3-triazol-2-yl)aniline (4) and 4-(1H-1,2,3-triazol-1-yl)aniline (4a)

To a stirred solution of compound 3 (2 g, 1 eq) in methanol (10 mL), PtO₂ (0.19 g) was added and stirred under hydrogen atmosphere (balloon pressure) at room temperature for overnight. The progress of the reaction was monitored by TLC. After completion of the reaction, the reaction mixture was filtered through celite. The filtrate was evaporated under reduced pressure. The crude product was purified by flash column chromatography on silica gel 100-200 mesh using 20% EtOAc-hexane to afford the title compounds 4 and 4a. Both the compounds were confirmed by NOE. Both: LCMS (m/z): 161.05 (M+1).

### Step-3: Synthesis of N-(4-(2H-1,2,3-triazol-2-yl)phenyl)-6-(1-methyl-3-(2-morpholinoethyl)-1H-indazol-6-yl)-2-morpholinopyrimidin-4-amine

The title compound has been synthesized by following the General procedure for Buchwald Coupling described above using compound 5 and amine 4 in Scheme 5. ¹H NMR (400 MHz, DMSO-d₆) δ: 9.67 (s, 1H), 8.17 (s, 1H), 8.08 (s, 4H), 8.03-7.82 (m, 3H), 7.73 (d, J=8.6 Hz, 1H), 6.74 (s, 1H), 4.05 (s, 3H), 3.83 (t, J=4.7 Hz, 4H), 3.74 (t, J=4.6 Hz, 4H), 3.63-3.55 (m, 4H), 3.09 (t, J=8.0 Hz, 2H), 2.71 (t, J=7.7 Hz, 2H), 2.48 (s, 4H); HPLC purity: 98.21%; LCMS Calculated for C₃₀H₃₄N₁₀O₂:566.29:observed: 567.50 (M+1).

### Examples 4-7

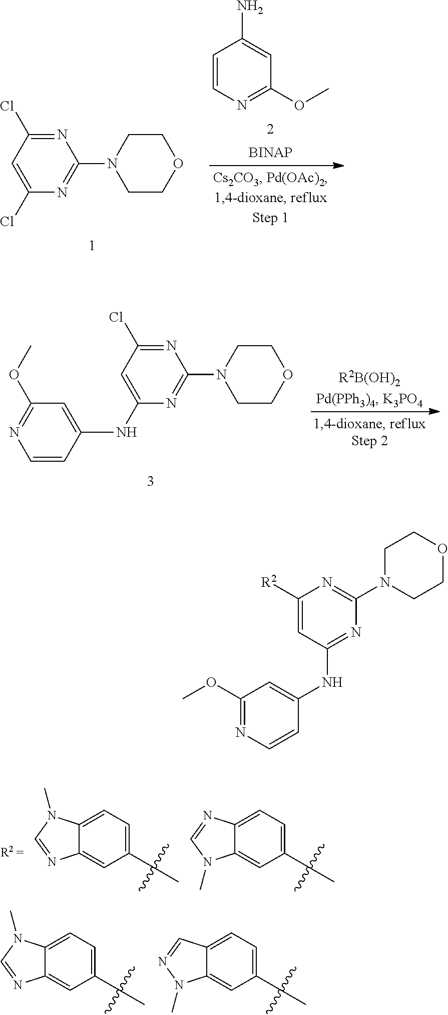

### Step 1: Synthesis of 6-chloro-N-(2-methoxypyridin-4-yl)-2-morpholinopyrimidin-4-amine (³)

A mixture of 4-(4,6-dichloropyrimidin-2-yl)morpholino 1 (0.3 g, 1 eq), 2-methoxy 4-amino pyridine 2 (0.168 g, 1 eq), cesium carbonate (0.437 g, 1.5 eq) in 1,4-dioxane (20 mL) was taken and purged with argon for 10 min, followed by the addition of BINAP (0.099 g, 0.2 eq) and purged argon for additional 5 min. Palladium acetate (0.04 g, 0.2 eq) was added and stirred at 100° C. for overnight. The progress of the reaction was monitored by TLC. After completion of the reaction, the reaction mixture was filtered through celite and evaporated to dryness. The residue was taken in ethyl acetate (50 mL), washed with water, brine, dried over anhydrous sodium sulfate and evaporated under reduced pressure. The crude product was purified by column chromatography on silica gel 100-200 mesh using 30% EtOAc-hexane to afford the desired product 3. LCMS (m/z): 322.20 (M+1).

### Step 2

N-(2-methoxypyridin-4-yl)-6-(1-methyl-1H-benzo[d]imidazol-5-yl)-2-morpholinopyrimidin-4-amine: The title compound has been synthesized by following the General Procedure for Suzuki Coupling described above using compound 3 in Scheme 6 and (1-Methyl-1H-benzimidazol-5-yl)boronic acid. ¹H NMR (MeOD, 400 MHz)δ: 9.50 (d, J=1.8 Hz, 1H), 8.60 (s, 1H), 8.44-8.36 (m, 1H), 8.13-8.03 (m, 3H), 7.55 (d, J=6.3 Hz, 1H), 6.98 (d, J=2.0 Hz, 1H), 4.20 (d, J=2.3 Hz, 6H), 4.00-3.95 (m, 4H), 3.85-3.82 (m, 4H); HPLC purity: 97.21%; LCMS Calculated for C₂₂H₂₃N₇O₂(free base): 417.19; observed: 418.20 (M+1).

N-(2-methoxypyridin-4-yl)-6-(1-methyl-1H-benzo[d]imidazol-6-yl)-2-morpholinopyrimidin-4-amine: The title compound has been synthesized by following the General Procedure for Suzuki Coupling described above using compound 3 in Scheme 6 and 1-Methyl-1H-benzoimidazole-6-boronic acid. ¹H NMR (MeOD, 400 MHz) δ: 9.52 (s, 1H), 8.67 (s, 1H), 8.36 (d, J=8.2 Hz, 1H), 8.15-8.06 (m, 2H), 7.99 (d, J=7.9 Hz, 1H), 7.60 (d, J=5.9 Hz, 1H), 7.02 (s, 1H), 4.26 (s, 3H), 4.20 (s, 3H), 4.00-3.95 (m, 4H), 3.90-3.86 (m, 4H); HPLC purity: 96.55%; LCMS Calculated for C₂₂H₂₃N₇O₂(Free base): 417.19; observed: 418.20 (M+1).

N-(2-methoxypyridin-4-yl)-6-(1-methyl-1H-indazol-5-yl)-2-morpholinopyrimidin-4-amine): The title compound has been synthesized by following the General Procedure for Suzuki Coupling described above using compound 3 in Scheme 6 and (1-methyl-1H-indazol-5-yl)boronic acid. ¹H NMR (DMSO-d₆, 400 MHz) δ: 9.76 (s, 1H), 8.47 (s, 1H), 8.19 (m, 1H), 8.03-7.98 (m, 2H), 7.73 (d, J=8.4 Hz, 1H), 7.25 (s, 1H), 7.18 (d, J=4.4 Hz, 1H), 6.70 (s, 1H), 4.08 (s, 3H), 3.82-3.74 (m, 11H); HPLC purity: 95.10%; LCMS Calculated for C₂₁H₂₃N₇O₂: 417.19; observed: 418.20 (M+1).

N-(2-methoxypyridin-4-yl)-6-(1-methyl-1H-indazol-6-yl)-2-morpholinopyrimidin-4-amine: The title compound has been synthesized by following the General Procedure for Suzuki Coupling described above using compound 3 in Scheme 6 and 1-methylindazole-6-boronic acid pinacol ester. ¹H NMR (DMSO-d6, 400 MHz) δ: 9.82 (s, 1H), 8.24 (s, 1H), 8.10 (d, J=1.0 Hz, 1H), 8.00 (d, J=5.7 Hz, 1H), 7.86 (d, J=8.5 Hz, 1H), 7.78 (dd, J=8.5, 1.4 Hz, 1H), 7.26 (d, J=1.8 Hz, 1H), 7.19 (dd, J=5.9, 1.9 Hz, 1H), 6.77 (s, 1H), 4.13 (s, 3H), 3.82 (s, 3H), 3.82-3.72 (m, 8H); HPLC purity: 98.69%, LCMS Calculated for C₂₂H₂₃N₇O₂:417.19; observed: 418.20 (M+1).

### Example 8

### Synthesis of N-(4-chlorophenyl)-6-(1H-indazol-6-yl)-2-morpholinopyrimidin-4-amine

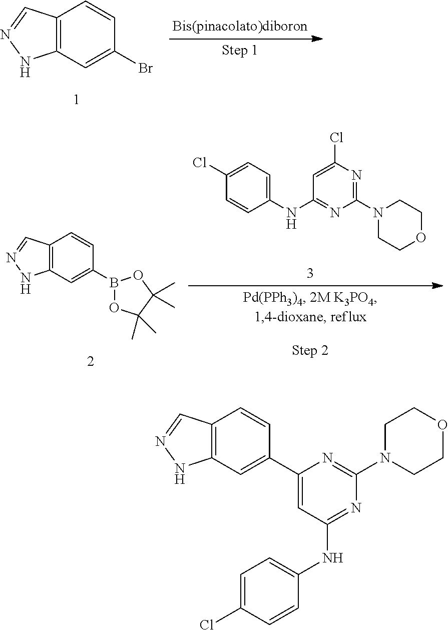 [SMILES: Brc1ccc2cn[nH]c2c1 ; CC1(C)OB(c2ccc3cn[nH]c3c2)OC1(C)C ; Clc1ccc(Nc2cc(Cl)nc(N3CCOCC3)n2)cc1 ; Clc1ccc(Nc2cc(-c3ccc4cn[nH]c4c3)nc(N3CCOCC3)n2)cc1]

### Step 1: Synthesis of 6-(4,4,5,5-tetramethyl-1,3,2-dioxaborolan-2-yl)-1H-indazole (2)

The title compound (crude product) has been synthesized by following the General Procedure for Boronate Ester Preparation described above using 6-bromo indazole 1 and bis(pinacolato)diboron. LCMS (m/z):245.05 (M+1).

### Step 2: Synthesis of N-(4-chlorophenyl)-6-(1H-indazol-6-yl)-2-morpholinopyrimidin-4-amine

The title compound has been synthesized by following the General Procedure for Suzuki Coupling described above using compound 3 and Boronate ester 2 in Scheme 7. ¹H NMR (400 MHz, DMSO-d₆) δ: 10.11 (bs, 1H), 8.15 (d, J=9.1 Hz, 2H), 7.90 (d, J=8.5 Hz, 1H), 7.76-7.70 (m, 2H), 7.64 (d, J=8.6 Hz, 1H), 7.41 (d, J=8.4 Hz, 2H), 6.71 (s, 1H), 3.84-3.78 (m, 4H), 3.76-3.70 (m, 4H); HPLC purity: 98.64%; LCMS Calculated for C₂₁H₁₉ClN₆O: 406.13; Observed: 407.15 (M+1).

### Example 9

Synthesis of N-(4-chlorophenyl)-6-(1H-indazol-5-yl)-2-morpholinopyrimidin-4-amine:

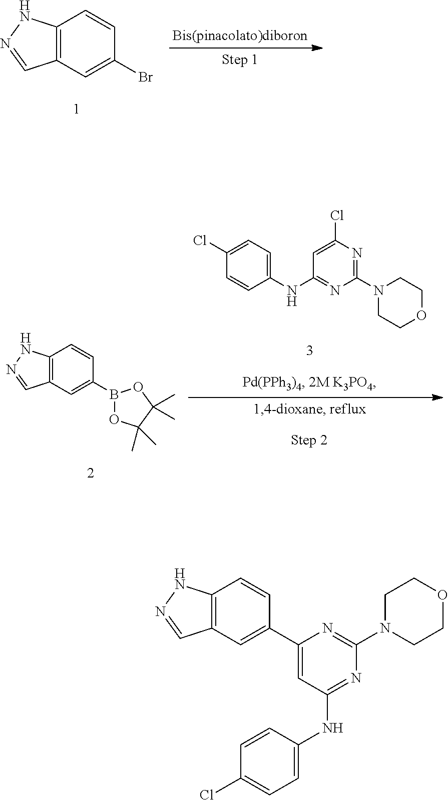 [SMILES: Brc1ccc2[nH]ncc2c1 ; Clc1ccc(Nc2cc(Cl)nc(N3CCOCC3)n2)cc1 ; CC1(C)OB(c2ccc3[nH]ncc3c2)OC1(C)C ; Clc1ccc(Nc2cc(-c3ccc4[nH]ncc4c3)nc(N3CCOCC3)n2)cc1]

### Step 1: Synthesis of 5-(4,4,5,5-tetramethyl-1,3,2-dioxaborolan-2-yl)-1H-indazole (2)

The title compound (crude product) has been synthesized by following the General Procedure for Boronate Ester Preparation described above using 5-bromo-1H-indazole 1 and bis(pinacolato)diboron. LCMS (m/z): 286.10 (M+1+CH₃CN).

### Step 2: Synthesis of N-(4-chlorophenyl)-6-(1H-indazol-5-yl)-2-morpholinopyrimidin-4-amine

The title compound has been synthesized by following the General Procedure for Suzuki Coupling described above using compound 3 and Boronate ester 2. ¹H NMR (400 MHz, DMSO-d₆) δ: 8.38 (s, 1H), 8.26 (s, 1H), 7.84 (d, J=8.7 Hz, 1H), 7.75-7.66 (m, 3H), 7.43 (d, J=8.6 Hz, 2H), 6.71 (s, 1H), 3.82-3.72 (m, 8H); HPLC purity: 98.77%; LCMS Calculated for C₂₁H₁₉ClN₆O: 406.13; Observed: 407.10 (M+1).

### Example 10

### Synthesis of 5-((6-(1H-indazol-6-yl)-2-morpholinopyrimidin-4-yl)amino)-N-methylpicolinamide

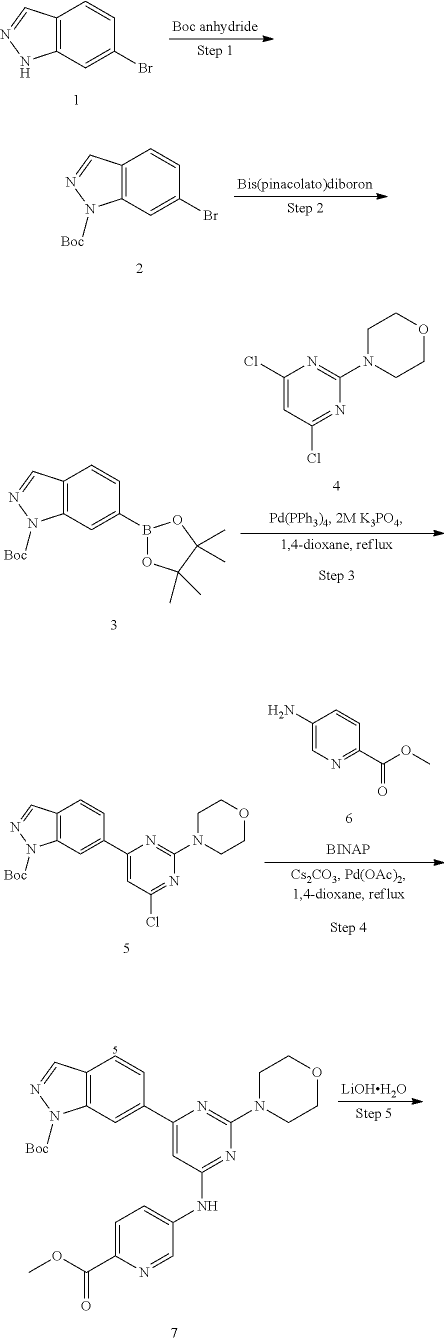

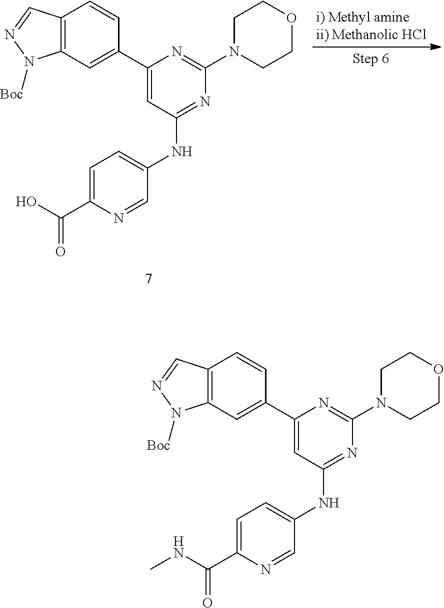 [SMILES: CC(C)(C)OC(=O)n1ncc2ccc(-c3cc(Nc4ccc(C(=O)O)nc4)nc(N4CCOCC4)n3)cc21 ; CNC(=O)c1ccc(Nc2cc(-c3ccc4cnn(C(=O)OC(C)(C)C)c4c3)nc(N3CCOCC3)n2)cn1]

### Step 1: Synthesis of tert-butyl 6-bromo-1H-indazole-1-carboxylate (2)

To a stirred solution of 6-bromo-1H-indazole 1 (0.9 g, 1 eq)in 1,4-dioxane (20 mL), 2 M NaOH solution (2.5 mL) was added and stirred at room temperature followed by the addition of Boc anhydride (1.106 g, 2 eq) and stirred for 30 min. The progress of the reaction was monitored by TLC. After completion of the reaction, the reaction mixture was quenched with water and extracted with ethyl acetate (2×50 mL). Combined organic extracts were washed with brine, dried over anhydrous sodium sulfate and concentrated under reduced pressure. The crude product was purified by column chromatography on silica gel 100-200 mesh using 15% EtOAc-hexane to afford the title compound 2. LCMS (m/z): 196.90 (M−Boc).

### Step 2: Synthesis of tert-butyl 6-(4,4,5,5-tetramethyl-1,3,2-dioxaborolan-2-yl)-1H-indazole-1-carboxylate(3)

The title compound has been synthesized by following the General Procedure for Boronate Ester Preparation described above using compound 2 and bis(pinacolato)diboron.

### Step 3: Synthesis of tert-butyl 6-(6-chloro-2-morpholinopyrimidin-4-yl)-1H-indazole-1-carboxylate (5)

The title compound has been synthesized by following the General Procedure for Suzuki Coupling described above using compound 4 and Boronate ester 3. LCMS (m/z): 316.10 (M−Boc).

### Step 4: Synthesis of tert-butyl 6-(6-((6-(methoxycarbonyl) pyridin-3-yl)amino)-2-morpholinopyrimidin-4-yl)-1H-indazole-1-carboxylate (7)

The title compound (crude) has been synthesized by following the General procedure for Buchwald Coupling described above using methyl 5-aminopicolinate 6 and compound 5.

### Step 5: Synthesis of 5-46-(1-(tert-butoxycarbonyl)-1H-indazol-6-yl)-2-morpholinopyrimidin-4-yl) amino)picolinic acid (8)

The title compound has been synthesized by following the General Procedure for Ester Hydrolysis described above using compound 7 and the crude product has been used as such for the next step. LCMS (m/z): 518.25 (M+1).

### Step 6: Synthesis of 5-((6-(1H-indazol-6-yl)-2-morpholinopyrimidin-4-yl)amino)-N-methylpicolinamide

The title compound has been synthesized by following the general procedure described above for Amide coupling by using compound 8 and methyl amine hydrochloride. The crude product has been stirred in methanolic HCl for 3 h and purified by preparative HPLC to afford the title compound. ¹H NMR (400 MHz, DMSO-d₆) δ: 10.52 (s, 1H), 8.95 (d, J=2.4 Hz, 1H),8.72 (s,1H), 8.28 (dd, J=2.4, 2.8 Hz, 1H), 8.17 (d, J=4 Hz, 2H), 8.05 (d, J=8.8 Hz, 1H), 7.91 (d, J=8.4 Hz, 1H), 7.66 (dd, J=1.2, 2.0 Hz, 1H), 6.82 (s, 1H), 3.82-3.73 (m, 8H), 2.80 (s, 3H); HPLC purity: 96.46%; LCMS Calculated for C₂₂H₂₂N₈O₂: 430.19; Observed: 431.25 (M+1).

### Examples 11-12

### Synthesis of N-(4-chlorophenyl)-6-(1-(cyclopropylmethyl)-1H-indazol-5-yl)-2-morpholino pyrimidin-4-amine and N-(4-chlorophenyl)-6-(2-(cyclopropylmethyl)-2H-indazol-5-yl)-2-morpholinopyrimidin-4-amine

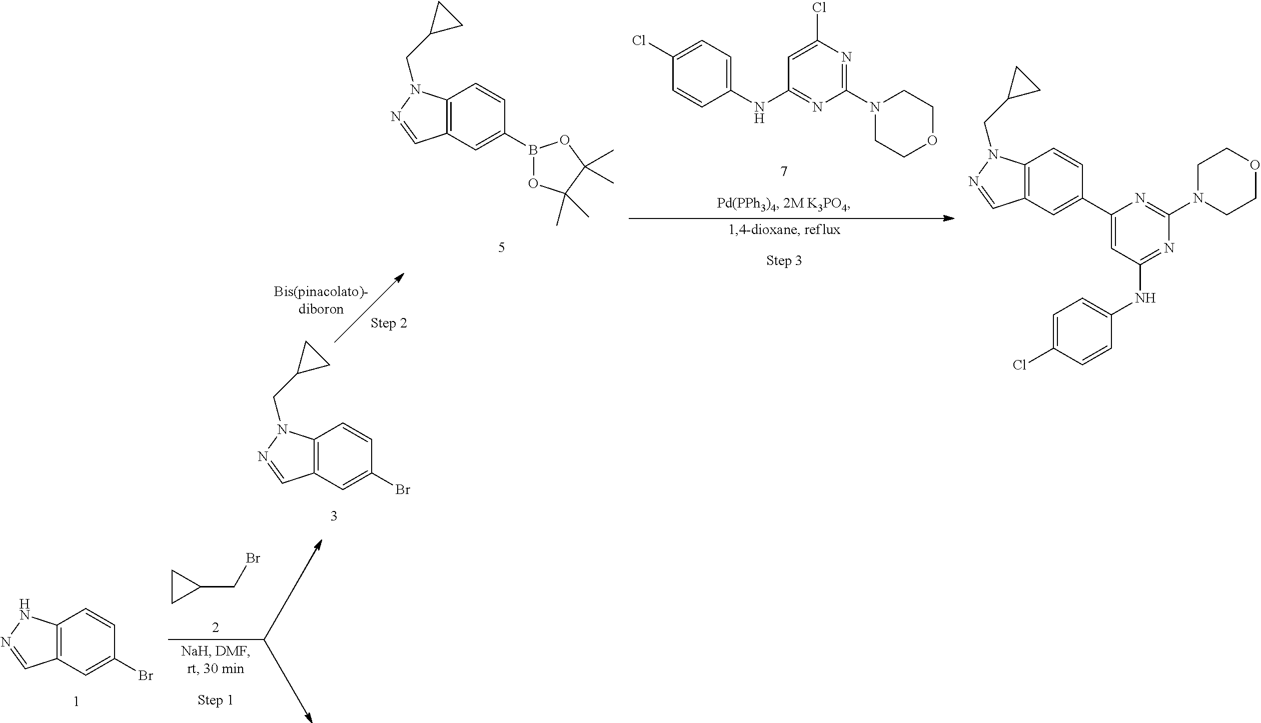 [SMILES: Brc1ccc2c(cnn2CC2CC2)c1 ; CC1(C)OB(c2ccc3c(cnn3CC3CC3)c2)OC1(C)C ; Clc1ccc(Nc2cc(Cl)nc(N3CCOCC3)n2)cc1 ; Clc1ccc(Nc2cc(-c3ccc4c(cnn4CC4CC4)c3)nc(N3CCOCC3)n2)cc1 ; BrCC1CC1 ; Brc1ccc2[nH]ncc2c1]

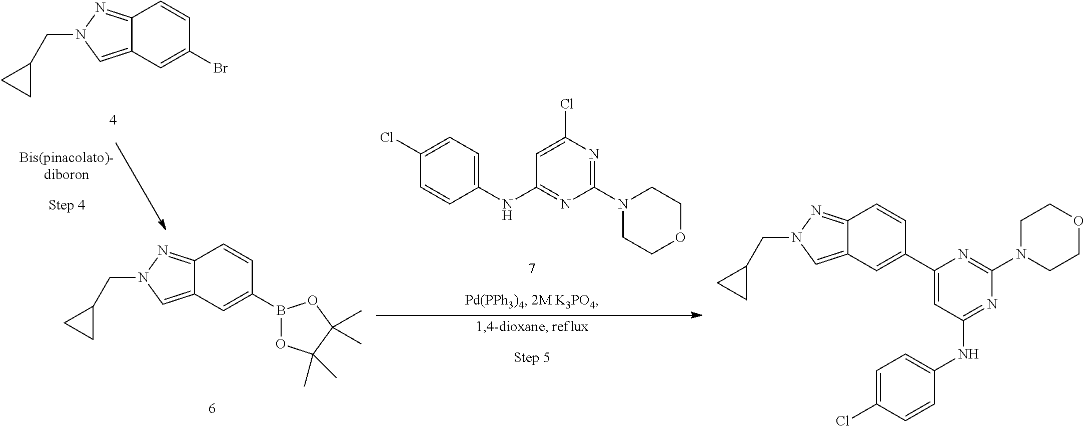 [SMILES: Brc1ccc2nn(CC3CC3)cc2c1 ; CC1(C)OB(c2ccc3nn(CC4CC4)cc3c2)OC1(C)C ; Clc1ccc(Nc2cc(Cl)nc(N3CCOCC3)n2)cc1 ; Clc1ccc(Nc2cc(-c3ccc4nn(CC5CC5)cc4c3)nc(N3CCOCC3)n2)cc1]

### Step 1: Synthesis of 5-bromo-1-(cyclopropylmethyl)-1H-indazole (3) and 5-bromo-2-(cyclopropylmethyl)-2H-indazole (4)

To a stirred solution of 5-bromo-1H-indazole 1 (2 g,1 eq) in DMF (20 mL), NaH (0.527 g, 1.3 eq) was added portion wise at 0° C. and stirred for 15 min followed by the addition of (bromomethyl)cyclopropane (2.05 g,1.5 eq). The reaction mixture was stirred at room temperature for 30 min. The progress of the reaction was monitored by TLC. After completion of the reaction, the reaction mixture was quenched with water, extracted with ethyl acetate (3×25 mL). Combined organic extracts were washed with brine, dried over anhydrous sodium sulfate and evaporated under reduced pressure. The crude product was purified by column chromatography on silica gel 100-200 mesh using 10% to 25% EtOAc-hexane to afford 1.6 g of compound 3 and 0.9 g of compound 4. LCMS (m/z): 250.95 (M+).

### Step 2: Synthesis of 1-(cyclopropylmethyl)-5-(4,4,5,5-tetramethyl-1,3,2-dioxaborolan-2-yl)-1H-indazole (5)

The title compound (crude product) has been synthesized by following the General Procedure for Boronate Ester Preparation described above using compound 3 and Bis (pinacolato)diboron. LCMS (m/z):299.15 (M+1).

### Step 3: Synthesis of N-(4-chlorophenyl)-6-(1-(cyclopropylmethyl)-1H-indazol-5-yl)-2-morpholino pyrimidin-4-amine

The title compound has been synthesized by following the General Procedure for Suzuki Coupling described above using compound 7 and Boronate ester 5. ¹H NMR (400 MHz, DMSO-d₆) δ: 9.93 (s, 1H), 8.40 (s, 1H), 8.23 (s, 1H), 7.92 (d, J=8.8 Hz, 1H), 7.84 (d, J=8.8 Hz, 1H), 7.71 (d, J=8.4 Hz, 2H), 7.41 (d, J=8.4 Hz, 2H), 6.65 (s, 1H), 4.36 (d, J=6.9 Hz, 2H), 3.84-3.77 (m, 4H), 3.75-3.69 (m, 4H), 1.32-1.26 (m, 1H), 0.55-0.37 (m, 4H); HPLC purity: 99.26%; LCMS Calculated for C₂₅H₂₅ClN₆O: 460.18; Observed: 461.15 (M+1).

### Step 4: Synthesis of 2-(cyclopropylmethyl)-5-(4,4,5,5-tetramethyl-1,3,2-dioxaborolan-2-yl)-2H-indazole (6)

The title compound (crude product) has been synthesized by following the General Procedure for Boronate Ester Preparation described above using bromo compound 4 and Bis (pinacolato)diboron. LCMS (m/z):299.15 (M+1).

### Step 5: Synthesis of N-(4-chlorophenyl)-6-(2-(cyclopropylmethyl)-2H-indazol-5-yl)-2-morpholinopyrimidin-4-amine

The title compound has been synthesized by following the General Procedure for Suzuki Coupling described above using compound 7 and Boronate ester 6. ¹H NMR (400 MHz, DMSO-d₆) δ:10.73 (s, 1H), 8.64 (s, 1H), 8.36 (s, 1H), 7.81-7.63 (m, 4H), 7.48-7.40 (m, 2H), 6.71 (s, 1H), 4.34 (d, J=7.2 Hz, 2H), 3.83-3.74 (m, 8H), 1.42-1.34 (m, 1H), 0.58-0.40 (m, 4H); HPLC purity: 99.77%; LCMS Calculated for C₂₅H₂₅ClN₆O: 460.18; Observed: 461.20 (M+1).

### Examples 13-14

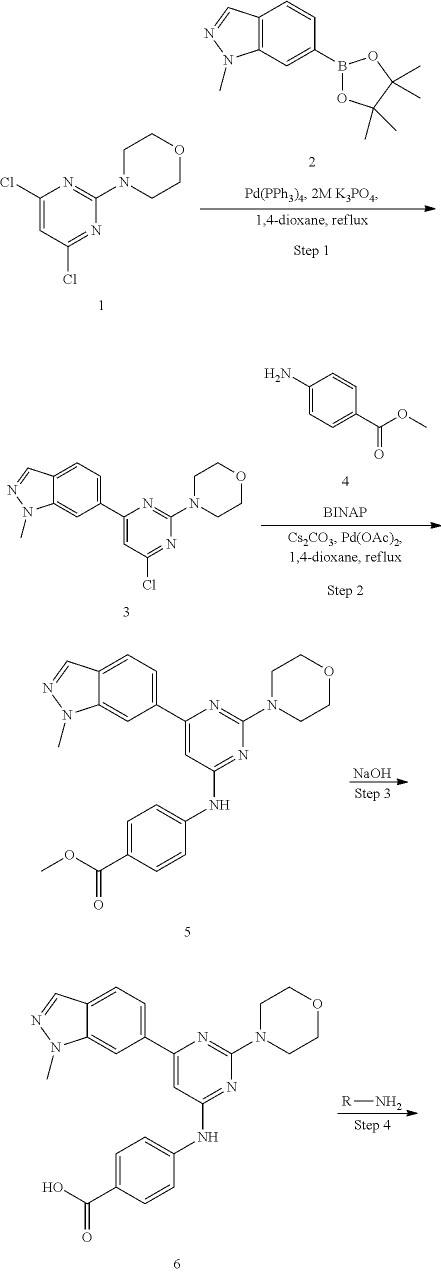

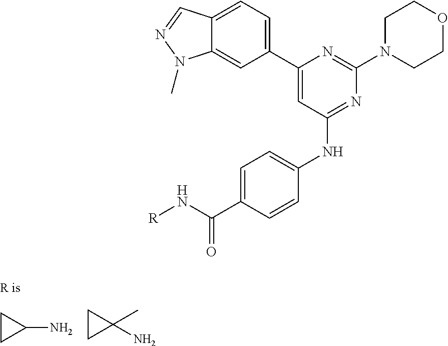

### Step 1: Synthesis of 4-(4-chloro-6-(1-methyl-1H-indazol-6-yl)pyrimidin-2-yl)morpholine (3)

The title compound has been synthesized by following the General Procedure for Suzuki Coupling described above using compound 1 and Boronate ester 2. LCMS (m/z): 330.05 (M+1).

### Step 2: Synthesis of methyl 4-((6-(1-methyl-1H-indazol-6-yl)-2-morpholinopyrimidin-4-yl)amino) benzoate (5)

The title compound (crude) has been synthesized by following the General procedure for Buchwald Coupling described above using compound 3 and methyl 4-aminobenzoate 4. LCMS (m/z): 445.20 (M+1).

### Step 3: Synthesis of 4-46-(1-methyl-1H-indazol-6-yl)-2-morpholinopyrimidin-4-yl)amino)benzoic acid (6)

The title compound has been synthesized by following the General Procedure for Ester Hydrolysis described above using compound 5 and the crude product has been used as such for the next step. LCMS (m/z): 431.25 (M+1).

Step 4: The following compounds were prepared using the above scheme.

N-cyclopropyl-4-((6-(1-methyl-1H-indazol-6-yl)-2-morpholinopyrimidin-4-yl)amino)benzamide: The title compound has been synthesized by following the general procedure described above for Amide coupling by using compound 6 and cyclopropyl amine. ¹H NMR (400 MHz, DMSO-d₆) 6:9.71 (s, 1H), 8.26 (d, J=13.3 Hz, 2H), 8.10 (s, 1H), 7.89-7.73 (m, 6H), 6.76 (s, 1H), 4.13 (s, 3H), 3.86-3.74 (m, 8H), 2.88-2.79 (m, 1H), 0.73-0.53 (m, 4H); HPLC purity: 96.95%; LCMS Calculated for C₂₆H₂₇N₇O₂: 469.22; Observed: 470.25 (M+1).

4-((6-(1-methyl-1H-indazol-6-yl)-2-morpholinopyrimidin-4-yl)amino)-N-(1-methyl cyclopropyl)benzamide: The title compound has been synthesized by following the general procedure described above for Amide coupling by using compound 6 and 1-methylcyclopropanamine. ¹H NMR (400 MHz, DMSO-d₆) δ: 9.67 (s, 1H), 8.47 (s, 1H), 8.24 (s, 1H), 8.10 (s, 1H), 7.89-7.71 (m, 6H), 6.75 (s, 1H), 4.13 (s, 3H), 3.82-3.72 (m, 8H), 1.36 (s, 3H), 0.75-0.70 (m, 2H), 0.61-0.57 (m, 2H); HPLC purity: 98.00%; LCMS Calculated for C₂₇H₂₉N₇O₂: 483.24; Observed: 484.30(M+1).

### Example 15

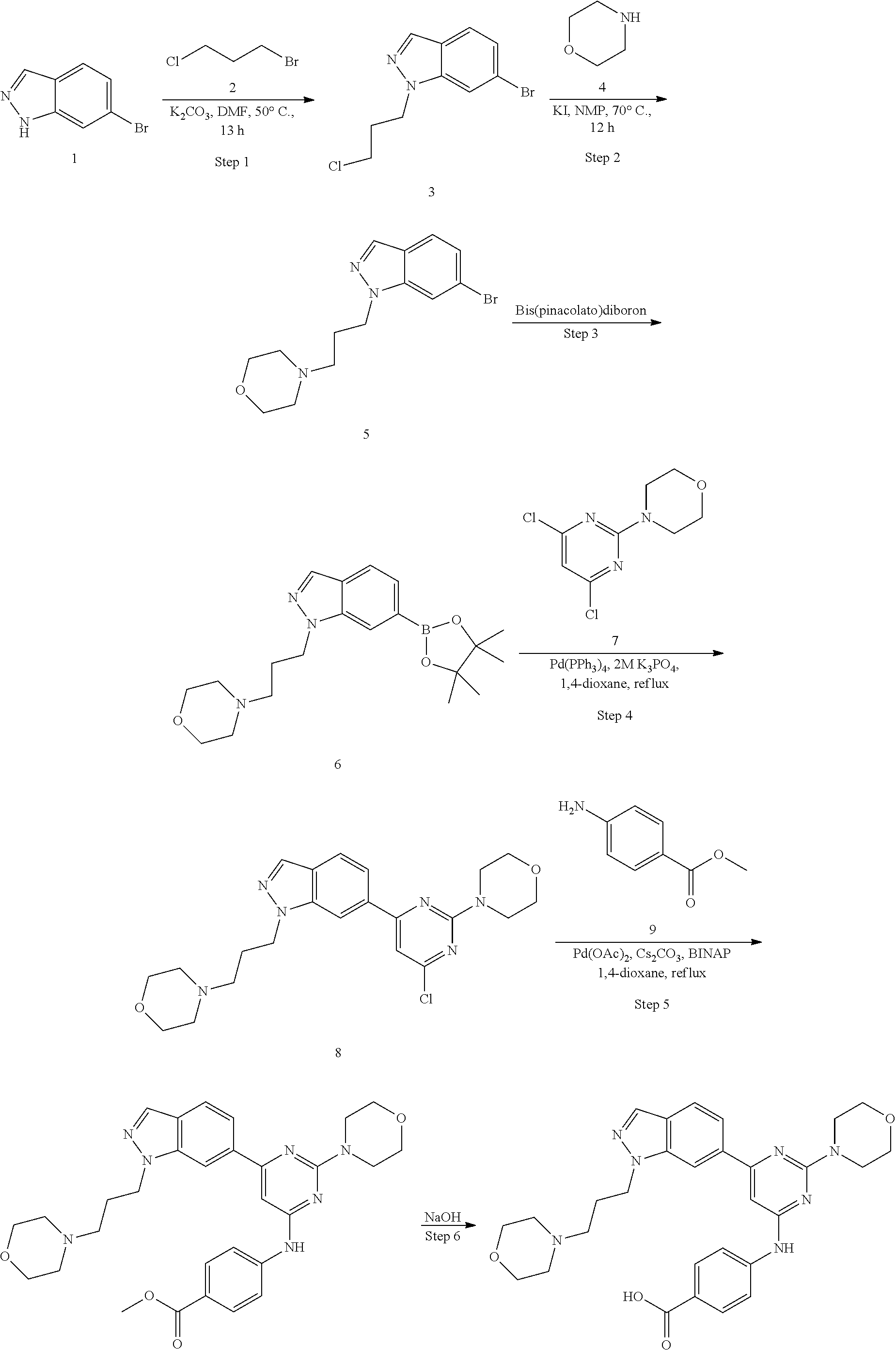

Methyl 4-((2-morpholino-6-(1-(3-morpholinopropyl)-1H-indazol-6-yl)pyrimidin-4-yl) amino)benzoate: ¹H NMR (400 MHz, DMSO-d₆) δ: 9.84 (s, 1H), 8.30 (s, 1H), 8.13 (s, 1H), 7.94 (d, J=8.5 Hz, 2H), 7.85 (d, J=8.9 Hz, 3H), 7.74 (d, J=8.5 Hz, 1H), 6.76 (s, 1H), 4.52 (t, J=6.6 Hz, 2H), 3.83-3.74 (m, 11H), 3.50 (d, J=5.0 Hz, 4H), 2.26-1.96 (m, 8H); HPLC purity: 95.24%; LCMS Calculated for C₃₀H₃₅N₇O₄: 557.28; Observed: 558.40 (M+1).

4-((2-Morpholino-6-(1-(3-morpholinopropyl)-1H-indazol-6-yl)pyrimidin-4-yl)amino) benzoic acid: ¹H NMR (400 MHz, DMSO-d₆) δ: 9.82 (s, 1H), 8.31 (s, 1H), 8.12 (s, 1H), 7.83 (ddd, J=39.7, 24.5, 8.4 Hz, 6H), 6.78 (s, 1H), 4.53 (t, J=6.2 Hz, 2H), 3.87-3.70 (m, 8H), 3.50 (d, J=9.7 Hz, 4H), 2.25-2.11 (m, 6H), 2.03 (dt, J=14.1, 7.5 Hz, 2H); HPLC purity: 98.3%; LCMS Calculated for C₂₉H₃₃N₇O₄: 543.26; Observed: 544.45 (M+1).

### Examples 16-17

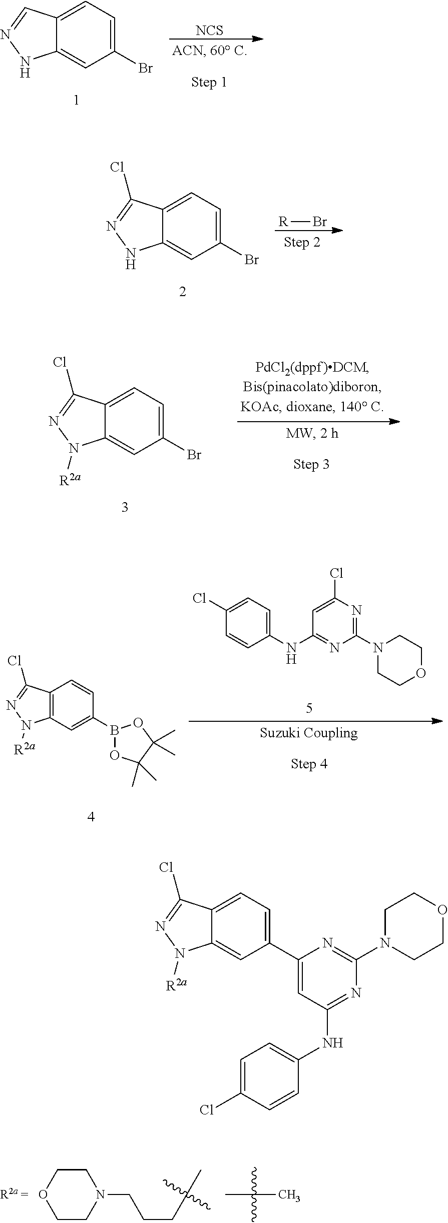

### Step 1: Synthesis of 6-bromo-3-chloro-1H-indazole (2)

To a stirred solution of compound 1 (4 g, 1 eq) in 40 mL acetonitrile, NCS (2.98 g, 1.1 eq) was added at room temperature and heated to 60° C. for 18 h. The progress of the reaction was monitored by TLC. After completion of the reaction, the reaction mixture was diluted with water and extracted with ethyl acetate. The combined organic extracts were dried over anhydrous sodium sulphate and evaporated under reduced pressure to afford title compound 2. LCMS (m/z):232.70 (M+2).

### Step 2: General Procedure for Alkylation

### i) Alkylation via 3-chloro-1-bromo propane (3)

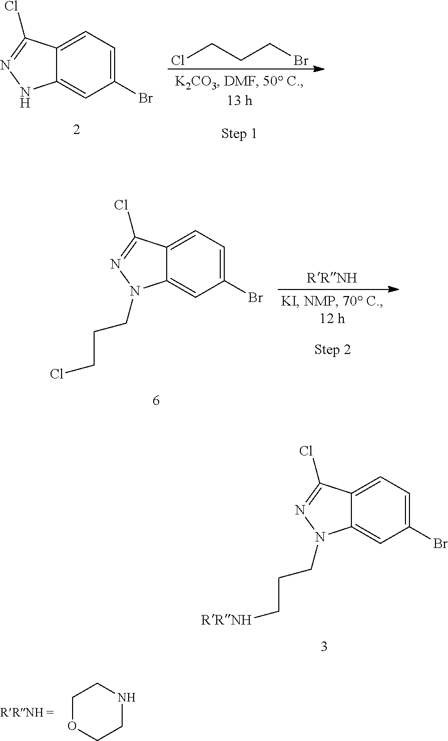

To a stirred solution of compound 2 (1 eq) in DMF, K₂CO₃ (3 eq) and 3-chloro-1-bromo propane (2 eq) were added and heated at 50° C. for 13 h. The progress of the reaction was monitored by TLC. After completion of the reaction, the reaction mixture was diluted with water and extracted with ethyl acetate. The combined organic extracts were evaporated under reduced pressure. The crude product was purified by column chromatography on silica gel 60-120 mesh using 20% hexane in ethyl acetate to afford compound 6.

To a stirred solution of compound 6 (1 eq) in NMP, KI (4 eq) and morpholine (6.2 eq) were added and heated at 70° C. for 12 h. After completion of the reaction, the reaction mixture was diluted with water and extracted with ethyl acetate. The combined organic extracts were evaporated under reduced pressure. The crude product was purified by column chromatography on silica gel 60-120 mesh using ethyl acetate/10% MeOH & DCM to afford 4-(3-(6-bromo-3-chloro-1H-indazol-1-yl)propyl)morpholine. LCMS (m/z): 360.15(M+1)

### ii) Alkylation Using DBU/ACN

To a stirred solution of compound 2 (1 eq) in acetonitrile, DBU (2 eq) was added followed by the addition of methyl iodide (1.2 eq). The reaction mixture was stirred at room temperature for 18 h. The progress of the reaction was monitored by TLC. After completion of the reaction, the reaction mixture was diluted with water and extracted with ethyl acetate. The combined organic extracts were dried over anhydrous sodium sulphate and evaporated under reduced pressure. The crude product was purified by column chromatography on silica gel 60-120 mesh using 20% ethyl acetate in n-hexane to afford 6-bromo-3-chloro-1-methyl-1H-indazole. ¹H NMR (400 MHz, DMSO-d₆) δ: 8.09 (s, 1H), 7.61 (d, J=8.8 Hz, 1H), 7.37-7.35 (m, 1H), 4.01 (s, 3H).

### Step 3: General Procedure for Boronate Ester Formation (4)

The title compounds were synthesized by following the General Procedure for Boronate Ester Preparation described above using the respective compounds 3 and bis(pinacolato)diboron to obtain compound 4.

| | LCMS: (m/z)/1H |
|---|---|
| Structure | NMR |
| 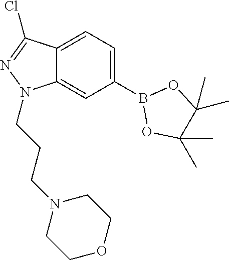 [SMILES: CC1(C)OB(c2ccc3c(Cl)nn(CCCN4CCOCC4)c3c2)OC1(C)C] | Monitored by TLC |
| 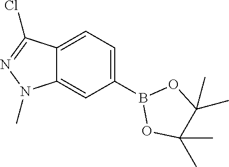 [SMILES: Cn1nc(Cl)c2ccc(B3OC(C)(C)C(C)(C)O3)cc21] | 293.15 (M + 1) |

### Step 4: General Procedure for Suzuki Coupling

The following compounds were prepared using the General Procedure for Suzuki Coupling described above using the respective pinacol boronates 4 and compound 5.

6-(3-chloro-1-(3-morpholinopropyl)-1H-indazol-6-yl)-N-(4-chlorophenyl)-2-morpholinopyrimidin-4-amine: ¹H NMR (400 MHz, DMSO-d₆) δ: 9.61 (s, 1H), 8.35 (s, 1H), 7.88-7.66 (m, 4H), 7.42-7.33 (m, 2H), 6.70 (s, 1H), 4.51 (t, J=6.4 Hz, 2H),3.82-3.70 (m, 8H), 3.47 (t, J=4.5 Hz, 4H), 2.25-2.12 (m, 6H), 2.01 (p, J=6.5 Hz, 2H); HPLC purity: 96.22%; LCMS Calculated for C₂₈H₃₁C₁₂N₇O₂ (Free base): 567.19; observed: 568.40 (M+1).

6-(3-chloro-1-methyl-1H-indazol-6-yl)-N-(4-chlorophenyl)-2-morpholinopyrimidin-4-amine: ¹H NMR (400 MHz, DMSO-d₆) δ: 9.56 (s, 1H), 8.25 (s, 1H), 7.81 (d, J=8.6 Hz, 1H), 7.70 (dd, J=17.2, 8.5 Hz, 3H), 7.34 (d, J=8.5 Hz, 2H), 6.67 (s, 1H), 4.07 (s, 3H), 3.80-3.68 (m, 8H); HPLC purity: 99.56%; LCMS Calculated for C₂₂H₂₀Cl₂N₆O (Free base): 454.11; Observed: 455.25 (M+1).

### Examples 18-21

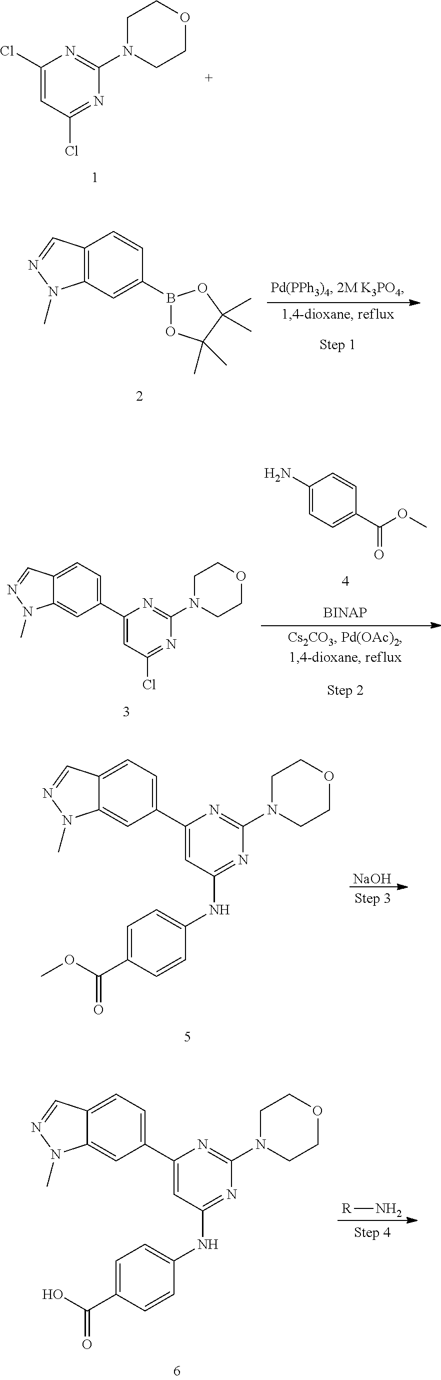

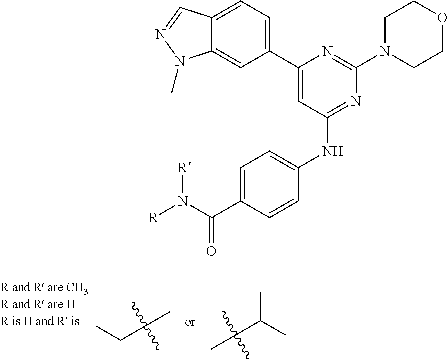

### Step 1: Synthesis of 4-(4-chloro-6-(1-methyl-1H-indazol-6-yl)pyrimidin-2-yl)morpholine (3)

The title compound has been synthesized by following the General Procedure for Suzuki Coupling described above using compound 1 and Boronate ester 2. LCMS (m/z): 330.05 (M+1).

### Step 2: Synthesis of methyl 4-((6-(1-methyl-1H-indazol-6-yl)-2-morpholinopyrimidin-4-yl)amino) benzoate (5)

The title compound (crude) has been synthesized by following the General procedure for Buchwald Coupling described above using compound 3 and methyl 4-aminobenzoate 4. ¹H NMR (400 MHz, DMSO-d₆) δ: 9.87 (s, 1H), 8.25 (s, 1H), 8.10 (s, 1H), 7.94 (d, J=8.4 Hz, 2H), 7.86 (d, J=8.2 Hz, 3H), 7.78 (d, J=8.5 Hz, 1H), 6.79 (s, 1H), 4.13 (s, 3H), 3.82-3.74 (m, 11H); HPLC purity: 94.79%; LCMS Calculated for C₂₄H₂₄N₆O₃: 444.19; Observed: 445.25 (M+1).

### Step 3: Synthesis of 4-((6-(1-methyl-1H-indazol-6-yl)-2-morpholinopyrimidin-4-yl)amino)benzoic acid (6)

The title compound has been synthesized by following the General Procedure for Ester Hydrolysis described above using compound 5 and the crude product has been used as such for the next step. ¹H NMR (400 MHz, DMSO-d₆) δ: 12.62 (s, 1H), 9.81 (s, 1H), 8.25 (s, 1H), 8.10 (s, 1H), 7.95-7.74 (m, 6H), 6.78 (t, 1H), 4.13 (s, 3H), 3.82-3.61 (m, 8H); HPLC purity: 90.18%; LCMS Calculated for C₂₃H₂₂N₆O₃: 430.18; Observed: 431.25 (M+1).

Step 4: The following compounds were prepared.

N,N-dimethyl-4-((6-(1-methyl-1H-indazol-6-yl)-2-morpholinopyrimidin-4-yl)amino) benzamide: The title compound has been synthesized by following the general procedure described above for Amide coupling by using compound 6 and N,N-dimethyl amine. ¹H NMR (400 MHz, MeOD) δ: 8.13 (d, J=14.5 Hz, 2H), 7.99 (d, J=8.4 Hz, 1H), 7.77 (d, J=8.1 Hz, 2H), 7.53 (dd, J=8.4, 5.8 Hz, 3H), 6.64 (s, 1H), 4.19 (s, 3H), 3.95-3.82 (m, 8H), 3.12 (s, 3H), 3.04 (s, 3H); HPLC purity: 97.24%; LCMS Calculated for C₂₅H₂₇N₇O₂(free base):457.22; observed: 458.30 (M+1).

4-((6-(1-methyl-4H-indazol-6-yl)-2-morpholinopyrimidin-4-yl)amino)benzamide: To a stirred solution of acid6 (100mg, 1 eq) in DMF (2 mL) CDI (45 mg, 1.2 eq) was added and the resulting mixture stirred at 60° C. for 1 h. Reaction mixture was cooled to room temperature, ammonium hydroxide (0.36 mL, 10 eq) was added and the reaction mixture was stirred for 1 h at room temperature. After completion of the reaction, water was added and extracted with ethyl acetate (2×25 mL). Combined organic layers were washed with brine, dried over anhydrous sodium sulfate and evaporated under reduced pressure. The crude product was purified by prep HPLC to afford the desired product. ¹H NMR (400 MHz, DMSO-d₆) δ: 10.19 (s, 1H), 8.24 (s, 1H), 8.13 (s, 1H), 7.89 (dd, J=8.6, 2.4 Hz, 4H), 7.81-7.74 (m, 2H), 7.69 (d, J=8.4 Hz, 1H), 7.23 (d, J=6.9 Hz, 1H), 6.79 (s, 1H), 4.14 (s, 3H), 3.84-3.75 (m, 8H); HPLC purity: 96.84%; LCMS Calculated forC₂₃H₂₃N₇O₂(free base): 429.19; observed: 430.25 (M+1).

N-ethyl-4-((6-(1-methyl-4H-indazol-6-yl)-2-morpholinopyrimidin-4-yl)amino)benzamide: The title compound has been synthesized by following the general procedure described above for Amide coupling by using compound 6 and ethylamine. ¹H NMR (400 MHz, DMSO-d₆) δ: 10.15 (s, 1H), 8.36 (t, J=5.6 Hz, 1H), 8.24 (s, 1H), 8.13 (s, 1H), 7.93-7.83 (m, 3H), 7.82-7.75 (m, 2H), 7.69 (d, J=8.5 Hz, 1H), 6.79 (s, 1H), 4.14 (s, 3H), 3.84-3.75 (m, 8H), 3.34-3.22 (m, 2H), 1.12 (t, J=7.2 Hz, 3H); HPLC purity: 97.67%; LCMS Calculated for C₂₅H₂₇N₇O₂(free base): 457.22; observed: 458.40 (M+1).

N-isopropyl-4-((6-(1-methyl4H-indazol-6-yl)-2-morpholinopyrimidin-4-yl)amino) benzamide:The title compound has been synthesized by following the general procedure described above for Amide coupling by using compound 6 and isopropyl amine. ¹H NMR (400 MHz, DMSO-d₆) δ: 9.70 (s, 1H), 8.24 (s, 1H), 8.12-8.03 (m, 2H), 7.85 (dd, J=8.6, 3.4 Hz, 3H), 7.77 (d, J=8.5 Hz, 3H), 6.76 (s, 1H), 4.16-4.03 (m, 4H), 3.83-3.74 (m, 8H), 1.16 (d, J=6.6 Hz, 6H); HPLC purity: 98.16%; LCMS Calculated for C₂₆H₂₉N₇O₂(free base): 471.24; observed: 472.40 (M+1).

### Examples 22-23

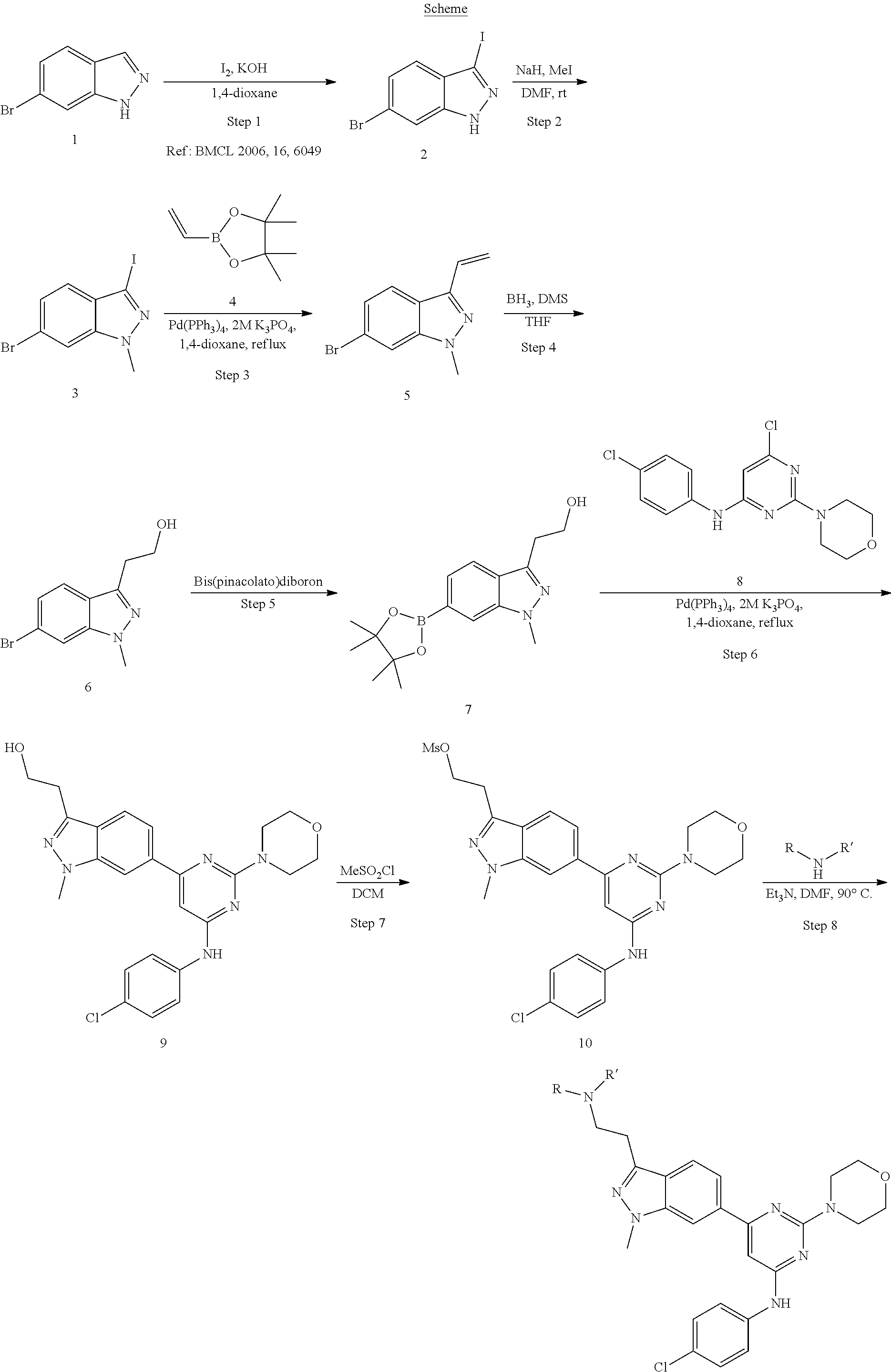

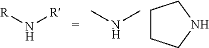

### Step 1: Synthesis of 6-bromo-3-iodo-1H-indazole (2)

To a stirred solution of 6-bromo-1H-indazole 1 (2 g, 1 eq) and 3N NaOH (20 mL) in 1,4-dioxane (40 mL), Iodine (5.67 g, 2.2 eq) was added and stirred at room temperature for 1 h. The progress of the reaction was monitored by TLC. After completion of the reaction, the reaction mixture was quenched with 20% citric acid solution, saturated sodium bicarbonate solution and extracted with ethyl acetate (3×50 mL). Combined organic extracts were washed with brine, dried over anhydrous sodium sulfate and evaporated under reduced pressure to afford the crude compound 2. LCMS (m/z): 323.05 (M+1).

### Step 2: Synthesis of 6-bromo-3-iodo-1-methyl-1H-indazole (3)

To a stirred solution of 6-bromo-3-iodo-1H-indazole 2 (3.2 g, 1 eq) in DMF (20 mL), NaH (0.59 g, 1.5 eq) was added and stirred at room temperature for 10 min followed by the addition of methyl iodide (2.8 g, 2 eq). The reaction mixture was stirred for 5 min at room temperature. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure. The crude product was purified by column chromatography on silica gel 100-120 mesh using 60% EtOAc-hexane to afford the title compound 3. LCMS (m/z): 336.95 (M+1).

### Step 3: Synthesis of 6-bromo-1-methyl-3-vinyl-1H-indazole (5)

The title compound has been synthesized by following the General Procedure for Suzuki Coupling described above using compound 3 and Boronate ester 4. LCMS (m/z): 239.05 (M+2).

### Step 4: Synthesis of 2-(6-bromo-1-methyl-1H-indazol-3-yl)ethanol (6)

To a stirred solution of compound 5 (1.9 g, 1 eq), in dry THF (40 mL), BH3:DMS (3.2 mL, 4 eq) was added at 0° C. and stirred at room temperature for 16 h. The progress of the reaction was monitored by TLC. After completion of the reaction, the reaction mixture was quenched with 3N NaOH and 30% H202 solution at 0° C. The reaction mixture was stirred at room temperature for 3 h and extracted with ethyl acetate (3×50 mL). Combined organic extracts were dried over anhydrous sodium sulfate and evaporated under reduced pressure. The crude product was purified by column chromatography on silica gel 100-120 mesh using 60% EtOAc-hexane to afford the title compound 6. LCMS (m/z): 255.05 (M+1).

### Step 5: Synthesis of 2-(1-methyl-6-(4,4,5,5-tetramethyl-1,3,2-dioxaborolan-2-yl)-1H-indazol-3-yl) ethanol (7)

The title compound (crude product) has been synthesized by following the General Procedure for Boronate Ester Preparation described above using bromo compound 6 and Bis (pinacolato) diboron. LCMS (m/z): 303.25 (M+1).

### Step 6: Synthesis of 2-(6-(6-((4-chlorophenyl)amino)-2-morpholinopyrimidin-4-yl)-1-methyl-1H-indazol-3-yl)ethanol (9)

The title compound has been synthesized by following the General Procedure for Suzuki Coupling described above using compound 8 and Boronate ester 7. LCMS (m/z): 465.25 (M+1).

### Step 7: Synthesis of 2-(6-(6-((4-chlorophenyl)amino)-2-morpholinopyrimidin-4-yl)-1-methyl-1H-indazol-3-yl)ethyl methanesulfonate (10)

To a stirred solution of compound 9 (0.25 g, 1 eq), in dichloromethane (10 mL), triethylamine (0.109 g, 2 eq) was added at room temperature and stirred at same temperature for 10 min followed by the addition of mesyl chloride (0.092 g, 1 eq) and stirred at same temperature for 1 h. The progress of the reaction was monitored by TLC. After completion of the reaction, the reaction mixture was diluted with dichloromethane (50 mL), washed with water, brine, dried over anhydrous sodium sulfate and evaporated under reduced pressure to afford the crude compound 4. LCMS (m/z): 543.30 (M+1).

### Step 8: General Procedure

To a stirred solution of compound 10 (1 eq), in DMF, triethylamine (2 eq) was added at room temperature and stirred at same temperature for 10 min followed by the addition of corresponding amine (2 eq) and stirred at 90° C. for 1 h. The progress of the reaction was monitored by TLC. After completion of the reaction, the reaction mixture was quenched with water and extracted with ethyl acetate. The combined organic extracts were dried over anhydrous sodium sulfate and evaporated under reduced pressure. The crude product was purified by column chromatography/preparative HPLC to afford the following compounds.

N-(4-chlorophenyl)-6-(3-(2-(dimethylamino)ethyl)-1-methyl-1H-indazol-6-yl)-2-morpholinopyrimidin-4-amine: ¹H NMR (400 MHz, MeOD) δ: 8.17 (d, J=1.3 Hz, 1H), 7.86-7.75 (m, 2H), 7.71-7.62 (m, 2H), 7.34-7.26 (m, 2H), 6.62 (s, 1H), 4.09 (s, 3H), 3.89-3.77 (m, 8H), 3.37-3.15 (m, 4H), 2.67 (s, 6H); HPLC purity: 96.71%; LCMS Calculated for C₂₆H₃₀ClN₇O: 491.22; observed: 492.40 (M+1).

N-(4-chlorophenyl)-6-(1-methyl-3-(2-(pyrrolidin-1-yl)ethyl)-1H-indazol-6-yl)-2-morpholino pyrimidin-4-amine: ¹H NMR (400 MHz, MeOD) δ: 8.10 (s, 1H), 8.03 (d, J=8.4 Hz, 1H), 7.65 (s, 2H), 7.58-7.51 (m, 1H), 7.46 (dd, J=8.6, 2.1 Hz, 2H), 6.63 (s, 1H), 4.16 (s, 3H), 3.93-3.70 (m, 12H), 3.51 (t, J=7.3 Hz, 2H), 3.26-3.22 (m, 2H), 2.20 (dq, J=11.2, 6.6, 5.2 Hz, 2H), 2.07 (dt, J=11.5, 5.7 Hz, 2H); HPLC purity: 98.81%; LCMS Calculated for C₂₈H₃₂ClN₇O (free base): 517.24; Observed: 518.40 (M+1).

### Examples 24-25

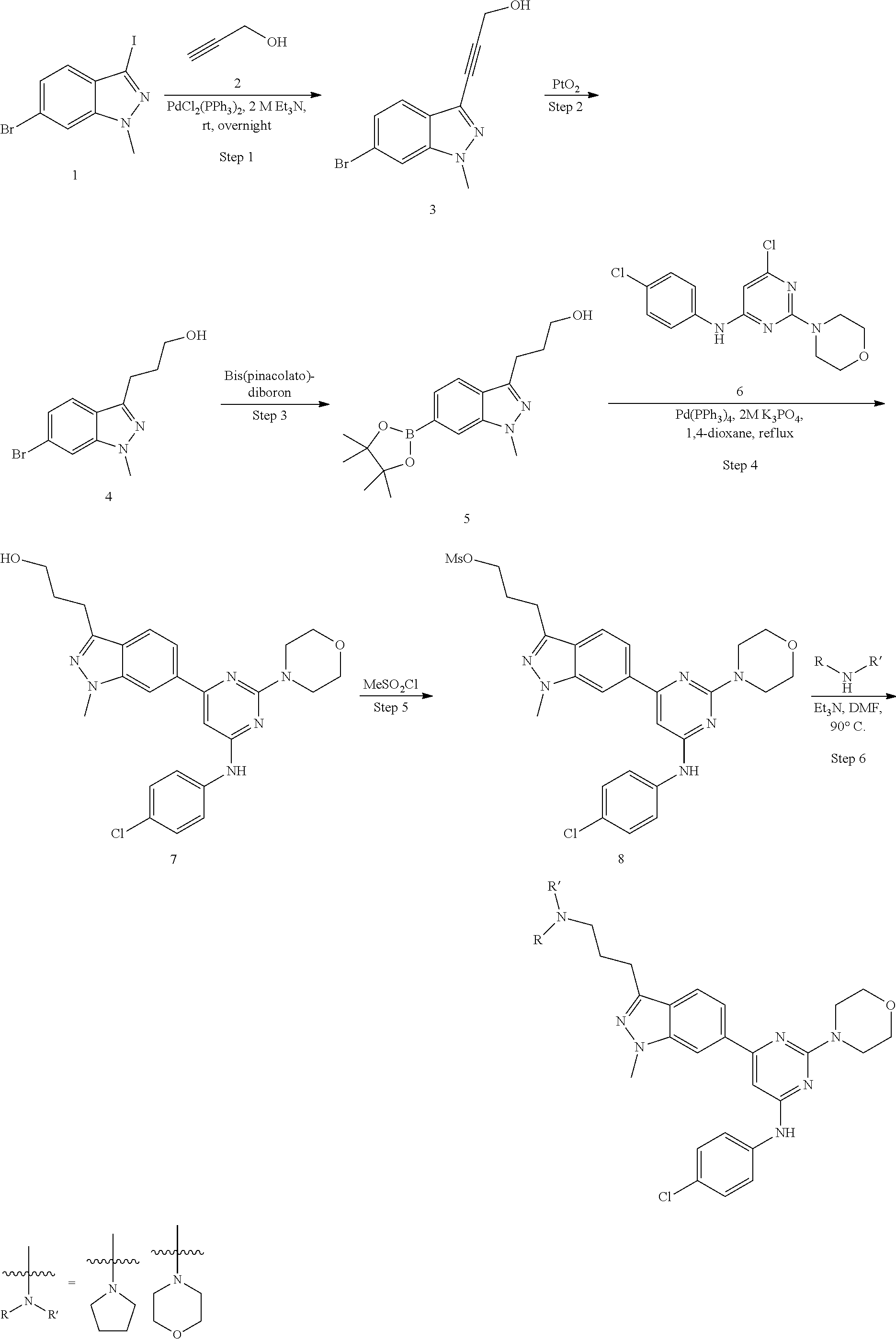

### Step 1: Synthesis of 3-(6-bromo-1-methyl-1H-indazol-3-yl)prop-2-yn-1-ol (3)

A stirred solution of 6-bromo-3-iodo-1-methyl-1H-indazole 1 (6 g, 1 eq), CuI (0.338 g, 0.1 eq) and Pd(PPh₃)₂Cl₂ (1.25 g, 0.1 eq) in triethylamine (50 mL) and stirred at room temperature for 15 min followed by the addition of prop-2-yn-1-ol (1.05 g, 1 eq) and stirred for 16 h at room temperature. The progress of the reaction was monitored by TLC. After completion of the reaction, the reaction mixture was concentrated under reduced pressure. The residue was purified by column chromatography on silica gel 100-200 mesh using 10% EtOAc-hexane to afford the title compound 3. LCMS (m/z): 267.05 (M+2).

### Step 2: Synthesis of 3-(6-bromo-1-methyl-1H-indazol-3-yl)propan-1-ol (4)

To a stirred solution of compound 3 (2 g, 1 eq), in ethanol (50 mL), PtO₂ (0.2 g) was added and stirred at room temperature under hydrogen atmosphere (balloon pressure) for 16 h. The progress of the reaction was monitored by TLC. After completion of the reaction, the reaction mixture was filtered through celite and evaporated under reduced pressure to afford the crude compound 4. LCMS (m/z): 269.05 (M+1).

### Step 3: Synthesis of 3-(1-methyl-6-(4,4,5,5-tetramethyl-1,3,2-dioxaborolan-2-yl)-1H-indazol-3-yl) propan-1-ol (5)

The title compound (crude product) has been synthesized by following the General Procedure for Boronate Ester Preparation described above using bromo compound 4 and Bis (pinacolato) diboron.

LCMS (m/z): 317.25 (M+1).

### Step 4: Synthesis of 3-(6-(6-((4-chlorophenyl)amino)-2-morpholinopyrimidin-4-yl)-1-methyl-1H-indazol-3-yl)propan-1-ol

The title compound has been synthesized by following the General Procedure for Suzuki Coupling described above using compound 6 and Boronate ester 5. ¹H NMR (400 MHz, DMSO-d₆) δ: 9.55 (s, 1H), 8.15 (s, 1H), 7.81 (d, J=8.4 Hz, 1H), 7.71 (td, J=6.1, 3.1 Hz, 3H), 7.41-7.29 (m, 2H), 6.69 (s, 1H), 4.52 (t, J=5.1 Hz, 1H), 4.04 (s, 3H), 3.83-3.68 (m, 8H), 3.49 (td, J=6.3, 4.9 Hz, 2H), 2.94 (t, J=7.7 Hz, 2H), 1.95-1.83 (m, 2H); HPLC purity: 96.49%; LCMS Calculated for C₂₅H₂₇ClN₆O₂: 478.19; Observed: 479.30 (M+1).

### Step 5: Synthesis of 3-(6-(6-((4-chlorophenyl)amino)-2-morpholinopyrimidin-4-yl)-1-methyl-1H-indazol-3-yl)propyl methanesulfonate (8)

To a stirred solution of compound 6 (0.3 g, 1 eq), in dichloromethane (5 mL), triethylamine (0.18 g, 2 eq) was added at room temperature and stirred at same temperature for 10 min followed by the addition of mesyl chloride (0.107 g, 1.5 eq) and stirred at same temperature for 1 h. The progress of the reaction was monitored by TLC. After completion of the reaction, the reaction mixture was quenched with water and extracted with 10% MeOH-DCM. Combined organic extracts were dried over anhydrous sodium sulfate and evaporated under reduced pressure to afford the crude compound 8. LCMS (m/z): 557.40 (M+1).

### Step 6: General Procedure

To a stirred solution of compound 8 (1 eq) in DMF, triethylamine (2 eq) was added at room temperature and stirred at same temperature for 10 min followed by the addition of corresponding amine (2 eq) and stirred at 90° C. for 1 h. The progress of the reaction was monitored by TLC. After completion of the reaction, the reaction mixture was quenched with water and extracted with ethyl acetate. The combined organic extracts were dried over anhydrous sodium sulfate and evaporated under reduced pressure. The crude product was purified by column chromatography/preparative HPLC to afford the following compounds.

N-(4-chlorophenyl)-6-(1-methyl-3-(3-(pyrrolidin-1-yl)propyl)-1H-indazol-6-yl)-2-morpholinopyrimidin-4-amine: ¹H NMR (400 MHz, MeOD) δ: 8.10-8.04 (m, 1H), 8.02-7.95 (m, 1H), 7.65 (s, 2H), 7.55-7.42 (m, 3H), 6.61 (s, 1H), 4.14 (s, 3H), 3.94-3.81 (m, 8H), 3.69 (dd, J=11.9, 5.4 Hz, 2H), 3.38-3.31 (m, 2H), 3.20-3.05 (m, 4H), 2.36-2.09 (m, 4H), 2.12-1.99 (m, 2H); HPLC purity: 96.27%; LCMS Calculated for C₂₉H₃₄ClN₇O (free base): 531.25; observed: 532.45 (M+1).

N-(4-chlorophenyl)-6-(1-methyl-3-(3-morpholinopropyl)-1H-indazol-6-yl)-2-morpholino pyrimidin-4-amine: ¹H NMR (400 MHz, MeOD) δ: 8.10-7.94 (m, 2H), 7.65 (d, J=7.9 Hz, 2H), 7.56-7.49 (m, 1H), 7.45 (dd, J=9.2, 2.7 Hz, 2H), 6.60 (d, J=2.8 Hz, 1H), 4.19-4.03 (m, 5H), 3.93-3.74 (m, 10H), 3.58-3.50 (m, 2H), 3.38-3.25 (m, 2H), 3.24-3.11 (m, 4H), 2.33 (ddd, J=12.0, 9.6, 6.2 Hz, 2H); HPLC purity: 98.58%; LCMS Calculated for C₂₉H₃₄ClN₇O₂ (free base): 547.25; observed: 548.45 (M+1).

### Example 26

### Synthesis of 6-(3-(3-aminopropyl)-1-methyl-1H-indazol-6-yl)-N-(4-chlorophenyl)-2-morpholino pyrimidin-4-amine

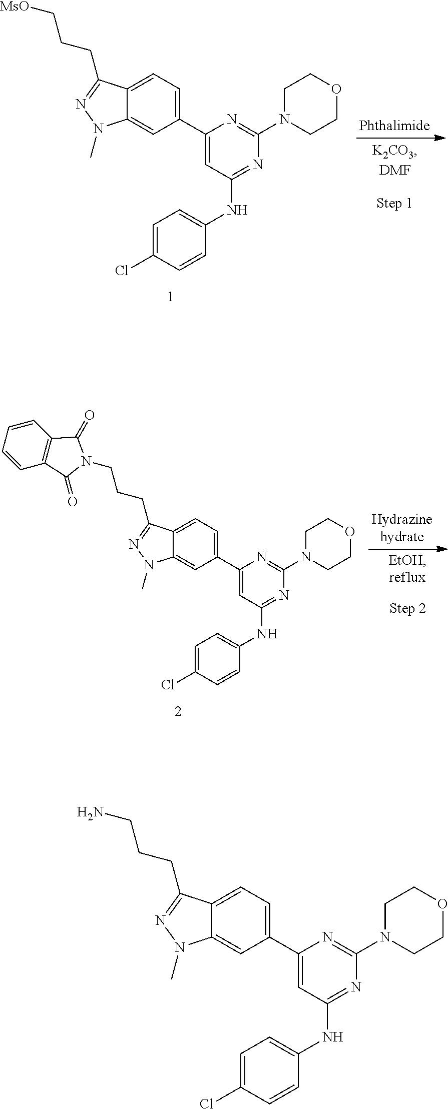 [SMILES: Cn1nc(CCCOS(C)(=O)=O)c2ccc(-c3cc(Nc4ccc(Cl)cc4)nc(N4CCOCC4)n3)cc21 ; Cn1nc(CCCN2C(=O)c3ccccc3C2=O)c2ccc(-c3cc(Nc4ccc(Cl)cc4)nc(N4CCOCC4)n3)cc21 ; Cn1nc(CCCN)c2ccc(-c3cc(Nc4ccc(Cl)cc4)nc(N4CCOCC4)n3)cc21]

### Step 1: Synthesis of 2-(3-(6-(6-((4-chlorophenyl)amino)-2-morpholinopyrimidin-4-yl)-1-methyl-1H-indazol-3-yl)propyl)isoindoline-1,3-dione (2)

To a stirred solution of compound 1 (0.2 g, 1 eq) in DMF (5 mL), K₂CO₃ (0.087 g, 1.5 eq) and phthalimide (0.092 g, 1.5 eq) were added and stirred at 80° C. for 18 h. The progress of the reaction was monitored by TLC. After completion of the reaction, the reaction mixture was diluted with water extracted with ethyl acetate (2×25 mL). Combined organic extracts were dried over anhydrous sodium sulfate and evaporated under reduced pressure to afford the crude product 2. LCMS (m/z): 608.50 (M+1).

### Step 2: Synthesis of 6-(3-(3-aminopropyl)-1-methyl-1H-indazol-6-yl)-N-(4-chlorophenyl)-2-morpholino pyrimidin-4-amine

To a stirred solution of compound 2 (0.3 g, 1 eq) in ethanol (10 mL), hydrazine monohydrate (0.049 g, 2 eq) was added and heated to reflux for 1.5 h. The reaction mixture was cooled to room temperature followed by the addition of 6 N HCl (5 mL) and heated to reflux for 30 min. The progress of the reaction was monitored by TLC. After completion of the reaction, the reaction mixture was quenched with 2N NaOH solution and extracted with 10% methanol in dichloromethane (3×25 mL). Combined organic extracts were dried over anhydrous sodium sulfate and evaporated under reduced pressure. The crude product was purified by preparative HPLC to afford the title compound. ¹H NMR (400 MHz, DMSO-d₆) δ: 9.56 (s, 1H), 8.15 (s, 1H), 7.82 (d, J=8.5 Hz, 1H), 7.75-7.67 (m, 3H), 7.41-7.32 (m, 2H), 6.69 (s, 1H), 4.04 (s, 3H), 3.83-3.68 (m, 8H), 2.94 (t, J=7.6 Hz, 2H), 2.60 (t, J=6.8 Hz, 2H), 1.82-1.76 (m, 2H); HPLC purity: 96.71%; LCMS Calculated for C₂₅H₂₈ClN₇O: 477.20; observed: 478.35 (M+1).

### Examples 27-35

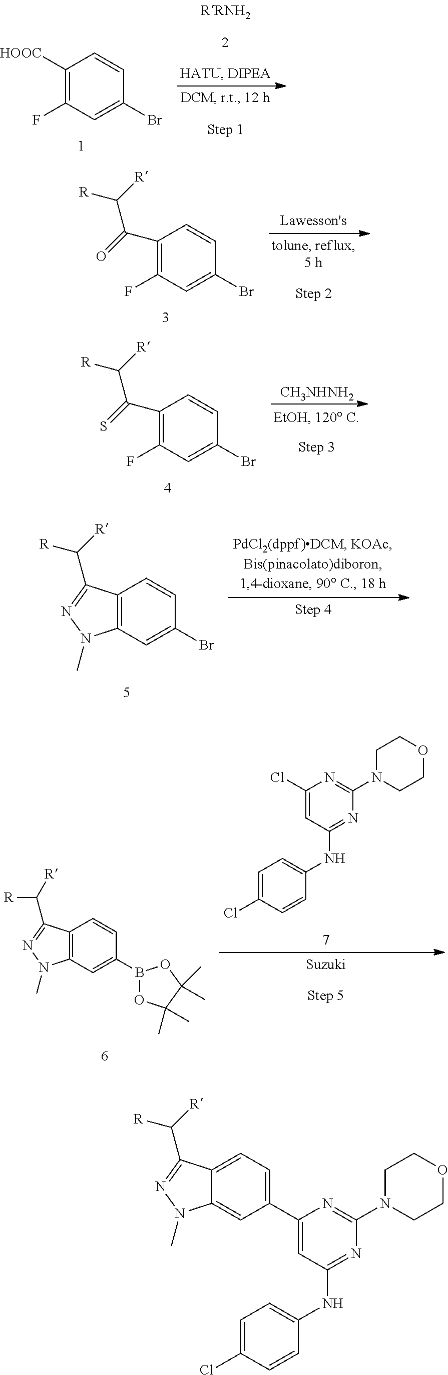

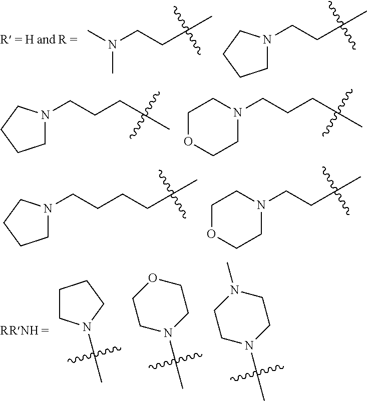

### Step 1: General Method for Peptide Coupling

To a stirred solution of 4-bromo-2-fluorobenzoic acid 1 (1 eq) in dichloromethane 40 mL amine 2 (1.2 eq), HATU (1.2 eq) and diisopropyl ethyl amine (3 eq) were added and stirred at room temperature for 12 h. The completion of the reaction was monitored by TLC. The reaction mixture was quenched with water and extracted with dichloromethane. The organic layers were washed with brine, dried over sodium sulfate, concentrated and purified to provide the following intermediates 3.

| Structure | LCMS (m/z) |
|---|---|
| 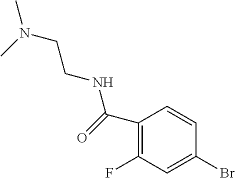 [SMILES: CN(C)CCNC(=O)c1ccc(Br)cc1F] | 289.05 (M + 1) |
| 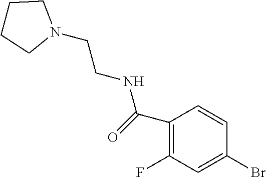 [SMILES: O=C(NCCN1CCCC1)c1ccc(Br)cc1F] | 315.10 (M + 1) |
| 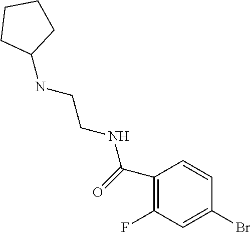 [SMILES: O=C(NCCNC1CCCC1)c1ccc(Br)cc1F] | 329.10 (M + 1) |
| 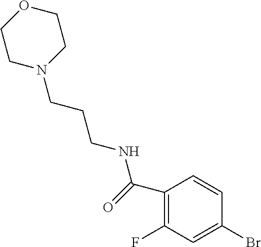 [SMILES: O=C(NCCCN1CCOCC1)c1ccc(Br)cc1F] | monitored by TLC |
| 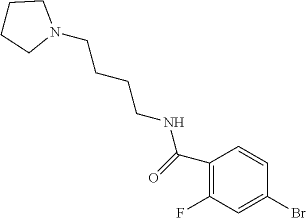 [SMILES: O=C(NCCCCN1CCCC1)c1ccc(Br)cc1F] | 343.05 (M + 1) |
| 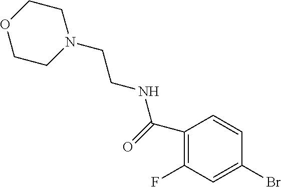 [SMILES: O=C(NCCN1CCOCC1)c1ccc(Br)cc1F] | 333.05 (M + 2) |
| 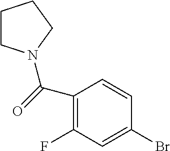 [SMILES: O=C(c1ccc(Br)cc1F)N1CCCC1] | 274.00 (M + 2) |
| 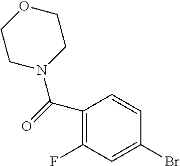 [SMILES: O=C(c1ccc(Br)cc1F)N1CCOCC1] | 288.00 (M + 1) |
| 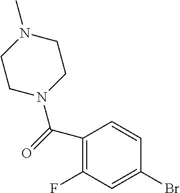 [SMILES: CN1CCN(C(=O)c2ccc(Br)cc2F)CC1] | 303.10 (M + 2) |

### Step 2: General Procedure for Thioamide Formation

To a stirred solution of compound 3 (1 eq) in toluene was added Lawesson's reagent (1 eq) and the reaction mixture was refluxed for 15 h. After completion of the reaction the reaction mixture was diluted with water and extracted with ethyl acetate. The combined organic fractions were washed with brine, dried over sodium sulfate and concentrated and purified by column chromatography to obtain the following intermediates 4.

| Structure | LCMS (m/z) |
|---|---|
| 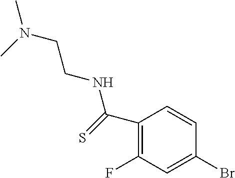 [SMILES: CN(C)CCNC(=S)c1ccc(Br)cc1F] | 305.05 (M + 1) |
| 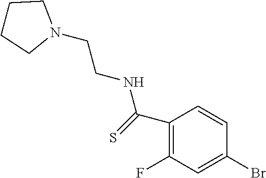 [SMILES: Fc1cc(Br)ccc1C(=S)NCCN1CCCC1] | 331.10 (M + 1) |
| 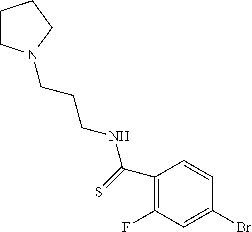 [SMILES: Fc1cc(Br)ccc1C(=S)NCCCN1CCCC1] | 345.15 (M + 1) |
| 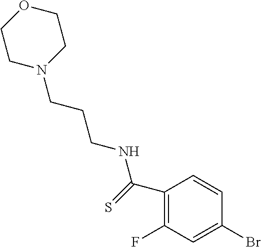 [SMILES: Fc1cc(Br)ccc1C(=S)NCCCN1CCOCC1] | monitored by TLC |
| 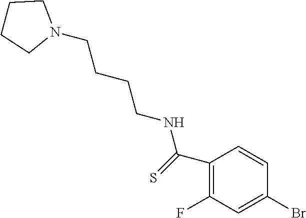 [SMILES: Fc1cc(Br)ccc1C(=S)NCCCCN1CCCC1] | 359.05 (M + 1) |
| 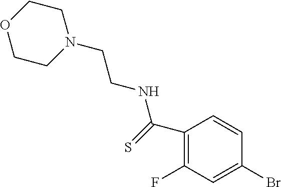 [SMILES: Fc1cc(Br)ccc1C(=S)NCCN1CCOCC1] | 349.05 (M + 2) |
| 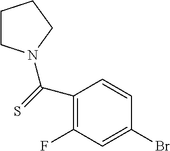 [SMILES: Fc1cc(Br)ccc1C(=S)N1CCCC1] | 288.00 (M + 1) |
| 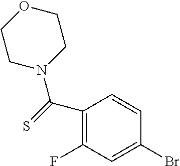 [SMILES: Fc1cc(Br)ccc1C(=S)N1CCOCC1] | 304.05 (M + 1) |
| 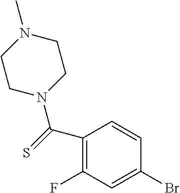 [SMILES: CN1CCN(C(=S)c2ccc(Br)cc2F)CC1] | 319.05 (M + 2) |

### Step 3: General Procedure for Cyclization Reaction

Compound 4 was dissolved in a 1:1 mixture of ethanol and methyl hydrazine and heated at 120° C. for 4 h. The completion of the reaction was monitored by TLC. The reaction mixture was concentrated in vacuo, washed with water and extracted with ethyl acetate. The combined ethyl acetate fraction were dried, concentrated and purified by column chromatography to obtain the following intermediates 5.

| Structure | LCMS (m/z) |
|---|---|
| 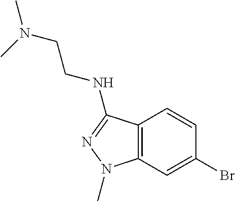 [SMILES: CN(C)CCNc1nn(C)c2cc(Br)ccc12] | 299.15 (M + 2) |
| 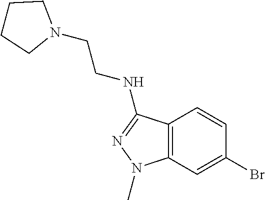 [SMILES: Cn1nc(NCCN2CCCC2)c2ccc(Br)cc21] | 323.10 (M + 1) |
| 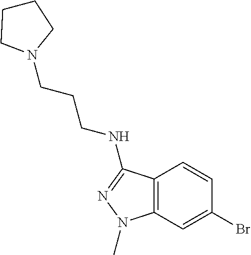 [SMILES: Cn1nc(NCCCN2CCCC2)c2ccc(Br)cc21] | 339.15 (M + 2) |
| 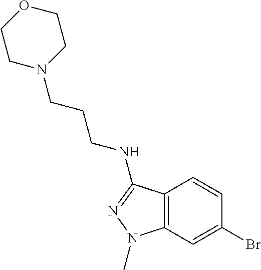 [SMILES: Cn1nc(NCCCN2CCOCC2)c2ccc(Br)cc21] | 353.15 (M + 1) |
| 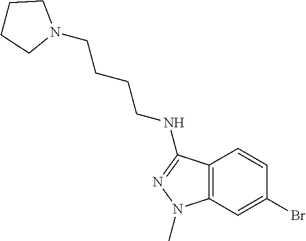 [SMILES: Cn1nc(NCCCCN2CCCC2)c2ccc(Br)cc21] | 353.10 (M + 2) |
| 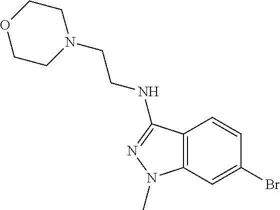 [SMILES: Cn1nc(NCCN2CCOCC2)c2ccc(Br)cc21] | 341.15 (M + 2) |
| 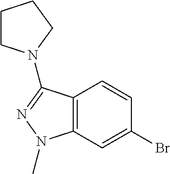 [SMILES: Cn1nc(N2CCCC2)c2ccc(Br)cc21] | 280.05 (M + 1) |
| 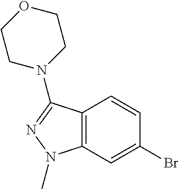 [SMILES: Cn1nc(N2CCOCC2)c2ccc(Br)cc21] | 297.05 (M + 2) |
| 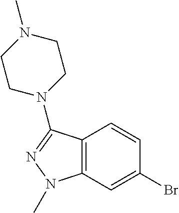 [SMILES: CN1CCN(c2nn(C)c3cc(Br)ccc23)CC1] | 309.10 (M + 1) |

Step 4: The following intermediates were prepared using the General procedure for Boronate ester preparation described above.

| Structure | Purity | LCMS (m/z) |
|---|---|---|
| 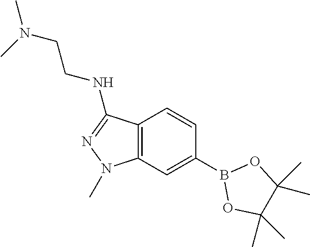 | 68% | 345.30 (M + 1) |
| 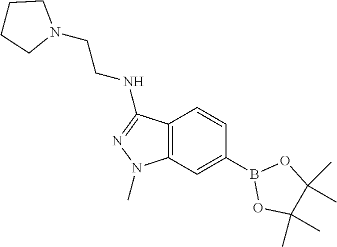 | Crude | 289.20 (M + 1, mass of boronic acid) |
| 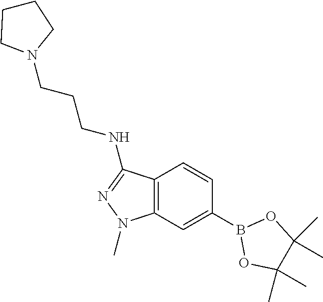 [SMILES: Cn1nc(NCCCN2CCCC2)c2ccc(B3OC(C)(C)C(C)(C)O3)cc21] | 87% | 385.45 (M + 1) |
| 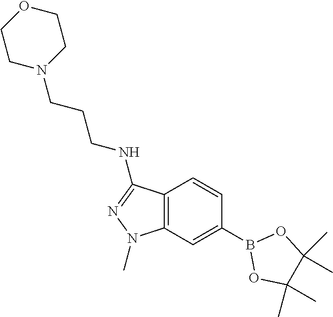 [SMILES: Cn1nc(NCCCN2CCOCC2)c2ccc(B3OC(C)(C)C(C)(C)O3)cc21] | Crude | 401.30 (M + 2) |
| 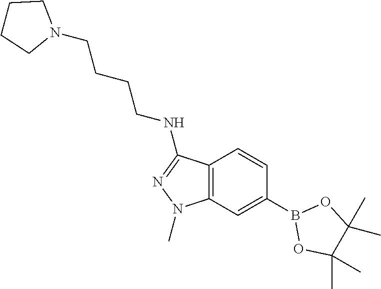 [SMILES: Cn1nc(NCCCCN2CCCC2)c2ccc(B3OC(C)(C)C(C)(C)O3)cc21] | 85% | 399.30 (M + 1) |
| 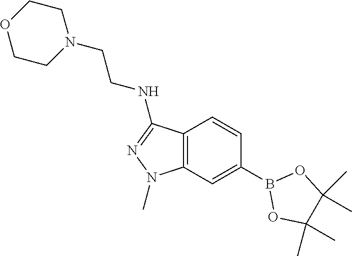 [SMILES: Cn1nc(NCCN2CCOCC2)c2ccc(B3OC(C)(C)C(C)(C)O3)cc21] | 78% | 387.35 (M + 1) |
| 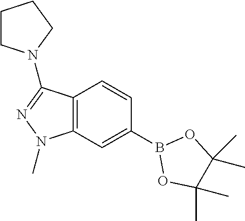 [SMILES: Cn1nc(N2CCCC2)c2ccc(B3OC(C)(C)C(C)(C)O3)cc21] | 79% | 328.21 (M + 1) |
| 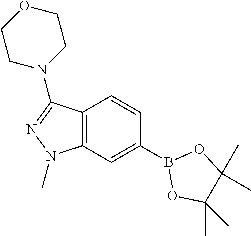 [SMILES: Cn1nc(N2CCOCC2)c2ccc(B3OC(C)(C)C(C)(C)O3)cc21] | 69% | 344.20 (M + 1) |
| 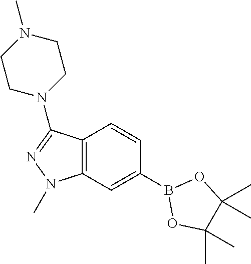 [SMILES: CN1CCN(c2nn(C)c3cc(B4OC(C)(C)C(C)(C)O4)ccc23)CC1] | 64% | 357.25 (M + 1) |

Step 5: The following compounds were prepared using the General Procedure for Suzuki Coupling described above.

N1-(6-(6-((4-chlorophenyl)amino)-2-morpholinopyrimidin-4-yl)-1-methyl-1H-indazol-3-yl)-N2,N2-dimethylethane-1,2-diamine: ¹H NMR (400 MHz, DMSO-d₆) δ: 9.53 (s, 1H), 7.91 (d, J=1.2 Hz, 1H), 7.78 (d, J=8.4 Hz, 1H), 7.76-7.67 (m, 2H), 7.52 (dd, J=8.5, 1.4 Hz, 1H), 7.41-7.32 (m, 2H), 6.66 (s, 1H), 5.97 (t, J=5.7 Hz, 1H), 3.85(s, 3H), 3.83-3.68 (m, 8H), 3.39-3.29 (m, 2H), 2.53-2.43 (m, 2H), 2.20 (s, 6H); HPLC purity: 95.32%; LCMS Calculated for C₂₆H₃₁ClN₈O: 506.23; Observed: 507.40 (M+1).

6-(6-((4-chlorophenyl)amino)-2-morpholinopyrimidin-4-yl)-1-methyl-N-(2-(pyrrolidin-1-yl)ethyl)-1H-indazol-3-amine: ¹H NMR (400 MHz, DMSO-d₆) δ: 9.52 (s, 1H), 7.91 (s, 1H), 7.78 (d, J=8.4 Hz, 1H), 7.75-7.67 (m, 2H), 7.52 (d, J=8.4 Hz, 1H), 7.41-7.31 (m, 2H), 6.65 (s, 1H), 6.03 (t, J=5.8 Hz, 1H), 3.81 (s,3H), 3.78-3.68 (m, 8H), 3.37 (q, J=6.5 Hz, 2H), 2.68 (t, J=6.8 Hz, 2H), 2.49-2.39 (m, 4H), 1.73-1.64 (m, 4H); HPLC purity: 99.1%; LCMS Calculated for C₂₈H₃₃ClN₈O: 532.25; Observed: 533.45 (M+1).

6-(6-((4-chlorophenyl)amino)-2-morpholinopyrimidin-4-yl)-1-methyl-N-(3-(pyrrolidin-1-yl)propyl)-1H-indazol-3-amine: ¹H NMR (400 MHz, MeOD) δ: 7.95 (s, 1H), 7.73-7.56 (m, 4H), 7.34-7.25 (m, 2H), 6.58 (s, 1H), 3.95-3.76 (m, 11H), 3.54-3.42 (m, 2H), 3.35-3.22 (m, 6H), 2.20-2.07 (m, 6H); HPLC purity: 95.47%; LCMS Calculated for C₂₉H₃₅ClN₈O (free base): 546.26; Observed: 547.45 (M+1).

6-(6-((4-chlorophenyl)amino)-2-morpholinopyrimidin-4-yl)-1-methyl-N-(3-morpholinopropyl)-1H-indazol-3-amine: ¹H NMR (400 MHz, DMSO-d₆; D₂O exchange) δ: 7.91-7.78 (m, 2H), 7.72-7.62 (m, 2H), 7.47-7.32 (m, 3H), 3.95 (d, J=12.7 Hz, 2H), 3.80-3.60 (m, 13H), 3.41 (d, J=12.3 Hz, 2H), 3.31 (t, J=6.6 Hz, 2H), 3.23-3.15 (m, 2H), 3.11-2.99 (m, 2H), 2.02-1.98 (m, 2H); HPLC purity: 95.95%; LCMS Calculated for C₂₉H₃₅ClN₈O₂ (free base): 562.26; Observed: 563.45 (M+1).

6-(6-((4-chlorophenyl)amino)-2-morpholinopyrimidin-4-yl)-1-methyl-N-(4-(pyrrolidin-1-yl)butyl)-1H-indazol-3-amine: ¹H NMR (400 MHz, DMSO-d₆) δ: 9.52 (s, 1H), 7.90 (s, 1H), 7.81-7.67 (m, 3H), 7.51 (d, J=8.5 Hz, 1H), 7.41-7.33 (m, 2H), 6.65 (s, 1H), 6.08 (t, J=5.7 Hz, 1H), 3.83-3.68 (m, 11H), 3.24 (q, J=6.5 Hz, 2H), 2.48-2.36 (m, 6H), 1.72-1.48 (m, 8H); HPLC purity: 99.3%; LCMS Calculated for C₃₀H₃₇ClN₈O: 560.28; Observed: 561.50 (M+1).

6-(6-((4-chlorophenyl)amino)-2-morpholinopyrimidin-4-yl)-1-methyl-N-(2-morpholinoethyl)-1H-indazol-3-amine: ¹H NMR (400 MHz, CD₃OD) δ: 7.95 (s, 1H), 7.74-7.57 (m, 4H), 7.36-7.25 (m, 2H), 6.58 (s, 1H), 3.94-3.76 (m, 16H), 3.66 (t, J=5.9 Hz, 2H), 3.15 (d, J=9.4 Hz, 2H), 3.04 (s, 3H); HPLC purity: 98.74%; LCMS Calculated for C₂₈H₃₃ClN₈O₂: 548.24; Observed: 549.50 (M+1).

N-(4-chlorophenyl)-6-(1-methyl-3-(pyrrolidin-1-yl)-1H-indazol-6-yl)-2-morpholinopyrimidin-4-amine: ¹H NMR (400 MHz, DMSO-d₆) δ: 10.20 (s, 1H), 7.96-7.60 (m, 2H), 7.72(d, J=8.4 Hz, 2H), 7.41 (d, J=8.4 Hz, 3H), 6.71 (s, 1H), 3.87 (s, 3H), 3.81-3.73 (m, 8H), 3.58 (q, J=6.6 Hz, 4H), 2.02-1.92 (m, 4H); HPLC purity: 97.70%; LCMS Calculated for C₂₆H₂₈ClN₇O (free base): 489.20; Observed: 490 (M+1).

N-(4-chlorophenyl)-6-(1-methyl-3-morpholino-1H-indazol-6-yl)-2-morpholinopyrimidin-4-amine: ¹H NMR (400 MHz, DMSO-d₆) δ: 9.56 (s, 1H), 8.07 (s, 1H), 7.88 (d, J=8.6 Hz, 1H), 7.75-7.67 (m, 2H), 7.58 (dd, J=8.6, 1.5 Hz, 1H), 7.41-7.32 (m, 2H), 6.68 (s, 1H), 3.93 (s, 3H), 3.76 (ddd, J=29.1, 6.9, 4.4 Hz, 12H), 3.31-3.22 (m, 4H); HPLC purity: 97.52%; LCMS Calculated for C₂₆H₂₈ClN₇O₂: 505.20; Observed: 506.35 (M+1).

N-(4-chlorophenyl)-6-(1-methyl-3-(4-methylpiperazin-1-yl)-1H-indazol-6-yl)-2-morpholinopyrimidin-4-amine: ¹H NMR (400 MHz, DMSO-d₆) δ: 10.66 (bs, 1H), 9.85 (s, 1H), 8.10 (s, 1H), 7.94 (d, J=8.6 Hz, 1H), 7.77-7.66 (m, 2H), 7.53 (dd, J=41.0, 8.7 Hz, 1H), 7.39 (d, J=8.7 Hz, 2H), 6.72 (s, 1H), 3.96 (d, J=7.8 Hz, 5H), 3.86-3.69 (m, 8H), 3.55-3.48 (m, 2H), 3.27 (t, J=8.7 Hz, 4H), 2.85 (d, J=4.5 Hz, 3H); HPLC purity: 93.58%; LCMS Calculated for C₂₇H₃₁ClN₈O: 518.23; Observed: 519.30 (M+1).

### Examples 36-38

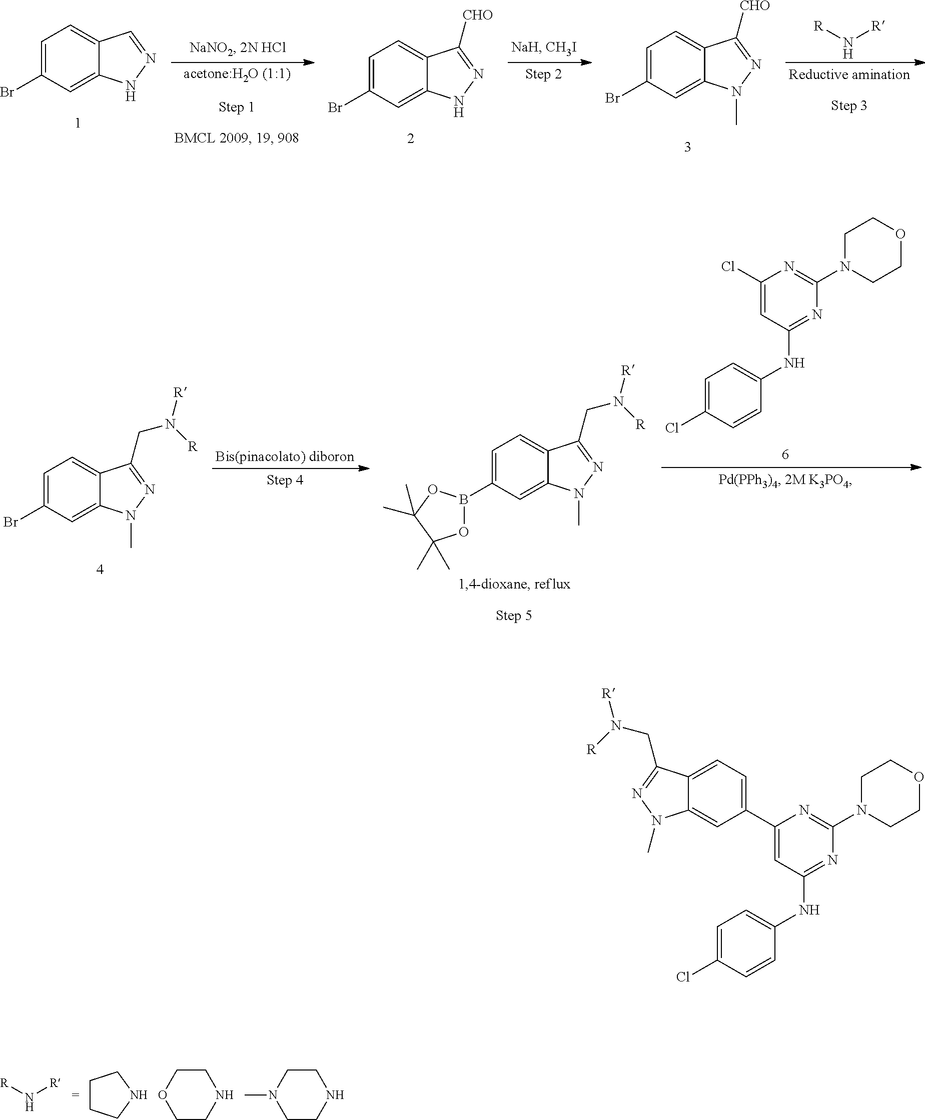

### Step 1: Synthesis of 6-bromo-1H-indazole-3-carbaldehyde (2)

To a stirred solution of sodium nitrite (8.44 g, 4.8 eq) in water:HCl (45:1; 460 mL), a solution of 6-bromo-1H-indole (5 g, 1 eq) in acetone (125 mL) was added and stirred at room temperature for 19 h. The progress of the reaction was monitored by TLC. After completion of the reaction, the reaction mixture was extracted diethyl ether and pentane. Combined organic extracts were washed with water, brine, dried over anhydrous sodium sulfate and evaporated under reduced pressure. The crude product was purified by column chromatography on silica gel 100-200 mesh using 20% EtOAc-hexane to afford the title compound 2. LCMS (m/z): 224.00 (M+).

### Step 2: Synthesis of 6-bromo-1-methyl1H-indazole-3-carbaldehyde (3)

To a stirred solution of 6-bromo-1H-indazole-3-carbaldehyde 2 (2 g, 1 eq) in DMF (15 mL), NaH (0.53 g, 1.5 eq) was added and stirred at room temperature for 10 min followed by the addition of methyl iodide(1.52 g, 2 eq). The reaction mixture was stirred at room temperature for 1 h. The progress of the reaction was monitored by TLC. After completion of the reaction, the reaction mixture was quenched with water and extracted with ethyl acetate (3×50 mL). Combined organic extracts were washed with brine, dried over anhydrous sodium sulfate and evaporated under reduced pressure. The crude compound was purified by column chromatography on silica gel 100-200 mesh using 10% EtOAc-hexane to afford the title compound 3. LCMS (m/z): 240.90 (M+2).

### Step 3: General Procedure for Reductive Amination

To a stirred solution of carbonyl compound (1 eq), and corresponding amine (1 eq) in MeOH was added acetic acid (catalytic) and stirred at room temperature for 30 min. Sodium cyanoborohydride (3 eq) was added to the reaction mixture and stirred for overnight. The progress of the reaction was monitored by TLC. After completion of the reaction, the reaction mixture was quenched with water and extracted using 15% MeOH:DCM. Combined organic extracts were dried over anhydrous sodium sulfate and evaporated under reduced pressure. The crude product has been purified by column chromatography on silica gel to afford the following intermediates.

| Structure (4) | LCMS (m/z) |
|---|---|
| 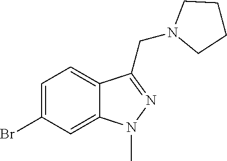 [SMILES: Cn1nc(CN2CCCC2)c2ccc(Br)cc21] | 294.10 (M + 1) |
| 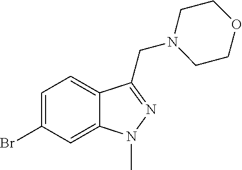 [SMILES: Cn1nc(CN2CCOCC2)c2ccc(Br)cc21] | 310.10 (M + 1) |
| 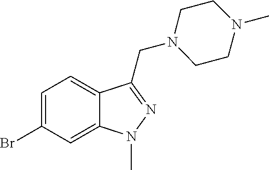 [SMILES: CN1CCN(Cc2nn(C)c3cc(Br)ccc23)CC1] | 323.15 (M + 1) |

Step 4: The following intermediates were prepared using the General Procedure for Boronate Ester Formation described above.

| Structure (5) | LCMS (m/z) |
|---|---|
| 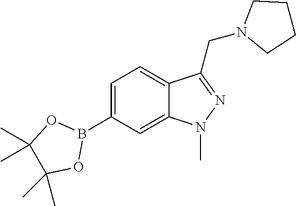 [SMILES: Cn1nc(CN2CCCC2)c2ccc(B3OC(C)(C)C(C)(C)O3)cc21] | 342.25 (M + 1) |
| 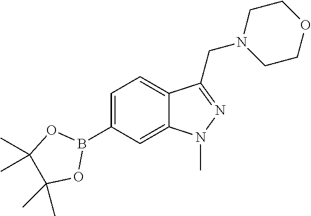 [SMILES: Cn1nc(CN2CCOCC2)c2ccc(B3OC(C)(C)C(C)(C)O3)cc21] | 358.20 (M + 1) |
| 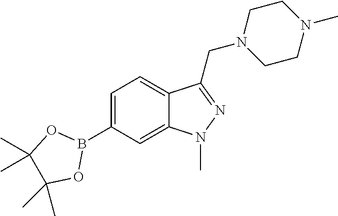 | 371.35 (M + 1) |

Step 5: The following compounds were prepared using the General procedure for Suzuki Coupling described above.

N-(4-chlorophenyl)-6-(1-methyl-3-(pyrrolidin-1-ylmethyl)-1H-indazol-6-yl)-2-morpholino pyrimidin-4-amine: ¹H NMR (400 MHz, MeOD) δ: 8.24-8.18 (m, 1H), 8.13 (d, J=8.6 Hz, 1H), 7.70-7.61 (m, 3H), 7.50-7.41 (m, 2H), 6.62 (d, J=7.8 Hz, 1H), 4.84 (d, J=13.4 Hz, 2H), 4.25 (s, 3H), 3.94-3.81 (m, 8H), 3.74-3.63 (m, 2H), 3.42-3.32 (m, 2H), 2.29-2.13 (m, 2H), 2.08 (ddd, J=12.7, 8.3, 4.4 Hz, 2H); HPLC purity: 98.04%; LCMS Calculated for C₂₇H₃₀ClN₇O (free base): 503.22; observed: 504.40 (M+1).

N-(4-chlorophenyl)-6-(1-methyl-3-(morpholinomethyl)-1H-indazol-6-yl)-2-morpholino pyrimidin-4-amine: ¹H NMR (400 MHz, MeOD) δ: 8.22 (s, 1H), 8.15 (d, J=8.5 Hz, 1H), 7.70-7.62 (m, 3H), 7.49-7.40 (m, 2H), 6.62 (s, 1H), 4.26 (s, 3H), 4.17-4.04 (m, 2H), 3.94-3.74 (m, 10H), 3.64-3.55 (m, 2H), 3.35-3.24 (m, 4H); HPLC purity: 92.69%; LCMS Calculated for C₂₇H₃₀ClN₇O₂ (free base): 519.21; observed: 520.40 (M+1).

N-(4-chlorophenyl)-6-(1-methyl-3-((4-methylpiperazin-1-yl)methyl)-1H-indazol-6-yl)-2-morpholinopyrimidin-4-amine: ¹H NMR (400 MHz, MeOD) δ: 8.18 (d, J=9.1 Hz, 2H), 7.69-7.57 (m, 3H), 7.46 (d, J=8.1 Hz, 2H),6.61 (s,1H), 4.63 (s, 2H), 4.23 (s, 3H), 3.93-3.83 (m, 8H), 3.62-3.50 (m, 8H), 2.97 (s, 3H); HPLC purity: 96.2%; LCMS Calculated for C₂₈H₃₃ClN₈O (free base): 532.25; observed: 533.45 (M+1).

### Example 39

### Synthesis of 4-((6-(1-methyl-1H-indazol-6-yl)-2-morpholinopyrimidin-4-yl)amino)benzonitrile

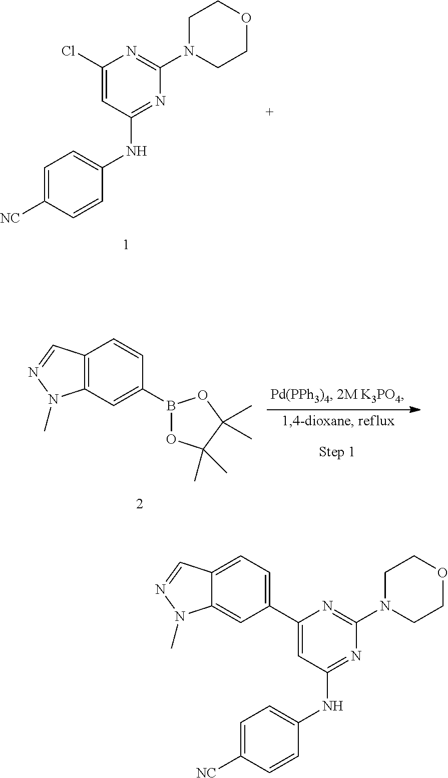 [SMILES: N#Cc1ccc(Nc2cc(Cl)nc(N3CCOCC3)n2)cc1 ; Cn1ncc2ccc(B3OC(C)(C)C(C)(C)O3)cc21 ; Cn1ncc2ccc(-c3cc(Nc4ccc(C#N)cc4)nc(N4CCOCC4)n3)cc21]

### Step 1: Synthesis of 4-((6-(1-methyl-1H-indazol-6-yl)-2-morpholinopyrimidin-4-yl)amino)benzonitrile

The title compound has been synthesized by following the General Procedure for Suzuki Coupling described above using chloro compound 1 and Boronate ester 2. ¹H NMR (400 MHz, DMSO-d₆) δ: 10.23-10.15 (m, 1H), 8.25 (s, 1H), 8.11 (s, 1H), 7.89 (dd, J=12.5, 8.6 Hz, 3H), 7.76 (t, J=8.8 Hz, 3H), 6.81 (s, 1H), 4.13 (s, 3H), 3.82-3.74 (m, 8H); HPLC purity: 97.77%; LCMS Calculated for C₂₃H₂₁N₇O: 411.18; Observed: 412.25 (M+1).

### Example 40

### Synthesis of 4-(6-(6-((4-chlorophenyl)amino)-2-morpholinopyrimidin-4-yl)-1-methyl-1H-indazol-3-yl)butan-1-ol

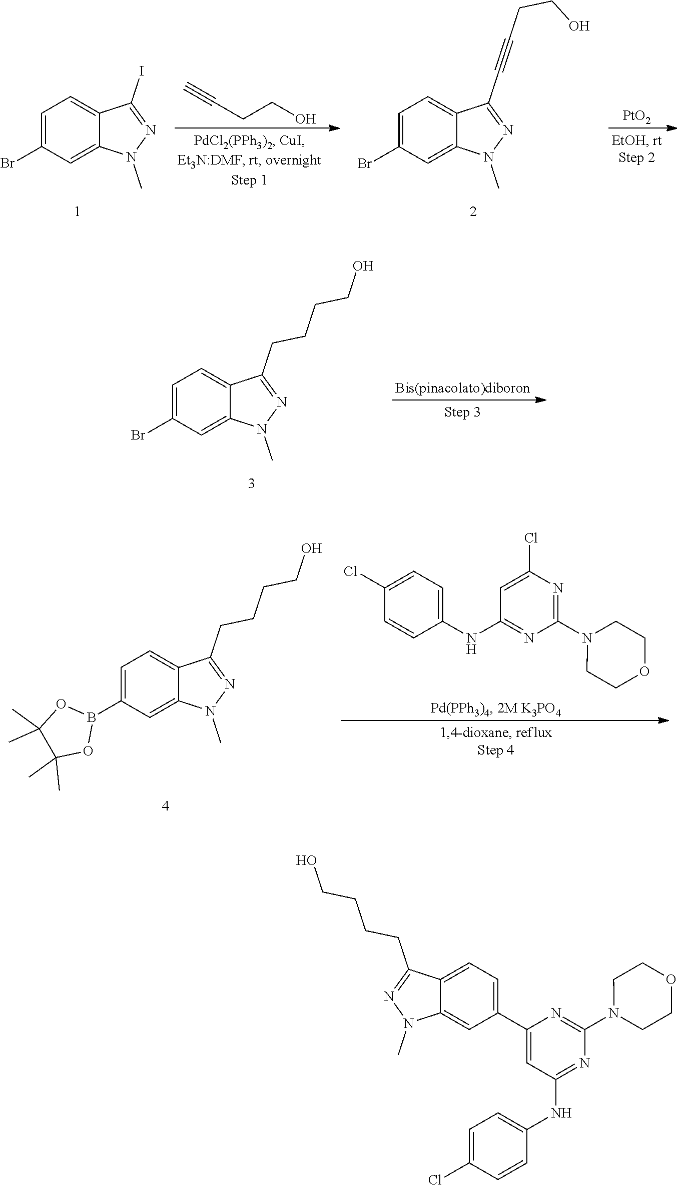

### Step 1: Synthesis of 4-(6-bromo-1-methyl-1H-indazol-3-yl)but-3-yn-1-ol (2)

A stirred solution of 6-bromo-3-iodo-1-methyl-1H-indazole 1 (2 g, 1 eq), CuI (0.112 g, 0.1 eq) and Pd(PPh3)₂Cl₂ (0.416 g, 0.1 eq) in triethylamine:DMF (1:1; 20 mL) and stirred at room temperature for 15 min followed by the addition of but-3-yn-1-ol (0.415 g, 1 eq) and stirred for 16 h at room temperature. The progress of the reaction was monitored by TLC. After completion of the reaction, the reaction mixture was concentrated under reduced pressure. The residue was purified by column chromatography on silica gel 100-200 mesh using 60% EtOAc-hexane to afford the title compound 2. LCMS (m/z): 279.10 (M+1).

### Step 2: Synthesis of 4-(6-bromo-1-methyl-1H-indazol-3-yl)butan-1-ol (3)

To a stirred solution of compound 2 (0.18 g, 1 eq), in ethanol (20 mL), PtO₂ (0.018 g) was added and stirred at room temperature under hydrogen atmosphere (balloon pressure) for 16 h. The progress of the reaction was monitored by TLC. After completion of the reaction, the reaction mixture was filtered through celite and evaporated under reduced pressure to afford the title compound 3. LCMS (m/z): 283.10 (M+1).

### Step 3: Synthesis of 4-(1-methyl-6-(4,4,5,5-tetramethyl-1,3,2-dioxaborolan-2-yl)-1H-indazol-3-yl) butan-1-ol (4)

The title compound (crude product) has been synthesized by following the General Procedure for Boronate Ester Preparation described above using bromo compound 3 and Bis (pinacolato) diboron. LCMS (m/z): 331.25 (M+1).

### Step 4: Synthesis of 4-(6-(6-((4-chlorophenyl)amino)-2-morpholinopyrimidin-4-yl)-1-methyl-1H-indazol-3-yl)butan-1-ol

The title compound has been synthesized by following the General Procedure for Suzuki Coupling described above using compound 5 and Boronate ester 4. ¹H NMR (400 MHz, DMSO-d₆) δ: 9.55 (s, 1H), 8.15 (s, 1H), 7.81 (d, J=8.5 Hz, 1H), 7.71 (td, J=6.7, 3.4 Hz, 3H), 7.44-7.30 (m, 2H), 6.69 (s, 1H), 4.37 (t, J=5.2 Hz, 1H), 4.04 (s, 3H), 3.83-3.68 (m, 8H), 3.43 (td, J=6.5, 5.1 Hz, 2H), 2.91 (t, J=7.5 Hz, 2H), 1.83-1.70 (m, 2H), 1.56-1.44 (m, 2H); HPLC purity: 97.74%; LCMS Calculated for C₂₆H₂₉ClN₆O₂: 492.20; Observed: 493.40 (M+1).

### Example 41

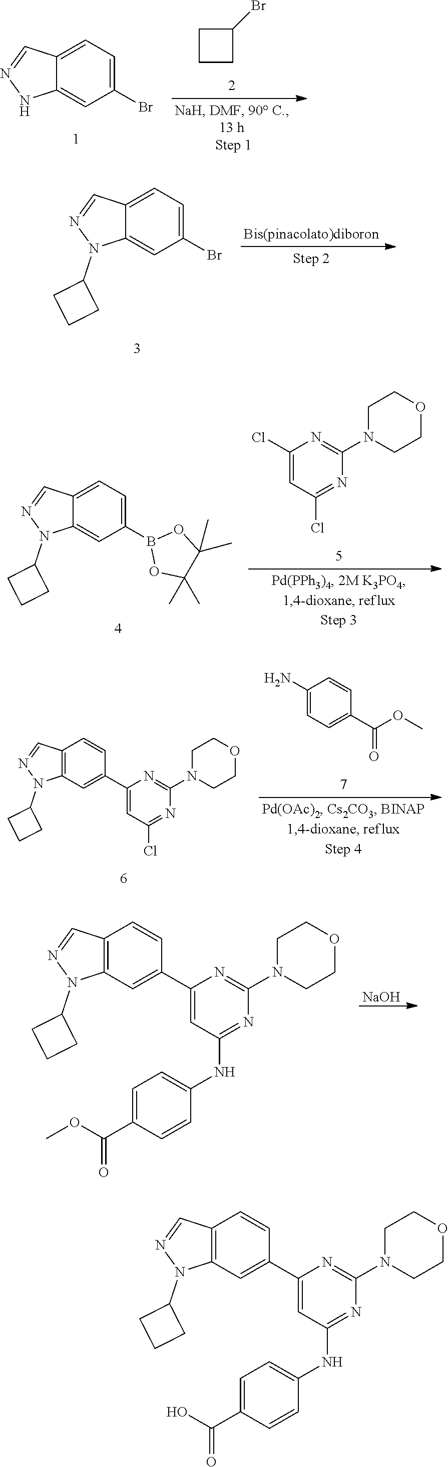

Methyl 4-((6-(1-cyclobutyl-1H-indazol-6-yl)-2-morpholinopyrimidin-4-yl)amino)benzoate: ¹H NMR (400 MHz, DMSO-d₆) δ: 9.85 (s, 1H), 8.28 (s, 1H), 8.18 (s, 1H), 7.94 (d, J=8.5 Hz, 2H), 7.85 (d, J=8.4 Hz, 3H), 7.76 (d, J=8.5 Hz, 1H), 6.77 (s, 1H), 5.42-5.38 (m, 1H), 3.86-3.71 (m, 11H), 2.74-2.59 (m, 2H), 2.50-2.40 (m, 2H), 1.91 (tt, J=10.2, 5.7 Hz, 2H); HPLC purity: 97.34%; LCMS Calculated for C₂₇H₂₈N₆O₃: 484.22; Observed: 485.30 (M+1).

4-((6-(1-cyclobutyl-1H-indazol-6-yl)-2-morpholinopyrimidin-4-yl) amino) benzoic acid: ¹H NMR (400 MHz, DMSO-d₆) δ: 9.80 (s, 1H), 8.28 (s, 1H), 8.17 (s, 1H), 7.95-7.73 (m, 6H), 6.78 (s, 1H), 5.42-5.38 (m, 1H), 3.82-3.74 (m, 8H), 2.74-2.59 (m, 4H), 2.01-1.85 (m, 2H); HPLC purity: 96.66%; LCMS Calculated for C₂₆H₂₆N₆O₃: 470.21; Observed: 471.35(M+1).

### Example 42

### Synthesis of N-methyl-4-((6-(1-methyl-1H-indazol-6-yl)-2-morpholinopyrimidin-4-yl)amino)benzene sulfonamide

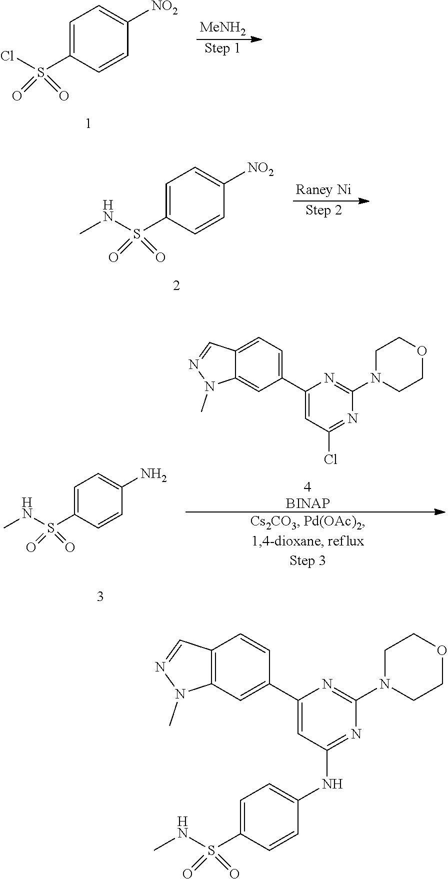

### Step 1: Synthesis of N-methyl-4-nitrobenzenesulfonamide (2)

To a stirred solution of 4-nitrobenzene-1-sulfonyl chloride 1 (1 g, 1 eq) in THF (10 mL), methyl amine 2M solution in THF (4.5 mL, 2 eq) was added at 0° C. and stirred at room temperature for 6 h. The progress of the reaction was monitored by TLC. After completion of the reaction, the reaction mixture was evaporated under reduced pressure. The crude product was purified by column chromatography on silica gel 100-200 mesh using 20% EtOAc-hexane to afford the title compound 2. ¹H NMR (400 MHz, DMSO-d₆) δ: 8.43 (d, J=9.2 Hz, 2H), 8.02 (d, J=9.2 Hz, 2H), 7.85 (s, 1H), 2.47 (d, J=3.6 Hz, 3H).

### Step 2: Synthesis of 4-amino-N-methylbenzenesulfonamide (3)

To a stirred solution of compound 2 (0.8 g, 1 eq) in methanol (20 mL), Raney nickel (1 g) was added and stirred at room temperature for 18 h under hydrogen atmosphere. The progress of the reaction was monitored by TLC. After completion of the reaction, the reaction mixture was filtered and evaporated under reduced pressure to afford the title compound 3. LCMS (m/z): 187.00 (M+1).

### Step 3: Synthesis of N-methyl-4-((6-(1-methyl-1H-indazol-6-yl)-2-morpholinopyrimidin-4-yl)amino) benzenesulfonamide

The title compound has been synthesized by following the general procedure described above for displacement reaction (IPA, Conc. HCl) by using chloro compound 4 and 4-amino-N-methyl benzenesulfonamide 3. ¹H NMR (400 MHz, DMSO-d₆) δ: 10.03 (s, 1H), 8.25 (q, J=1.0 Hz, 1H), 8.11 (d, J=1.0 Hz, 1H), 7.96-7.84 (m, 3H), 7.75 (td, J=7.7, 7.0, 1.6 Hz, 3H), 7.26 (s, 1H), 6.79 (s, 1H), 4.14 (s, 3H), 3.84-3.75 (m, 8H), 2.41 (d, J=4.1 Hz, 3H); HPLC purity: 96.2%; LCMS Calculated for C₂₃H₂₅N₇O₃S: 479.17; Observed: 480.25 (M+1).

### Example 43

### Synthesis of N-(4-(4H-1,2,4-triazol-3-yl)phenyl)-6-(1-methyl-1H-indazol-6-yl)-2-morpholinopyrimidin-4-amine

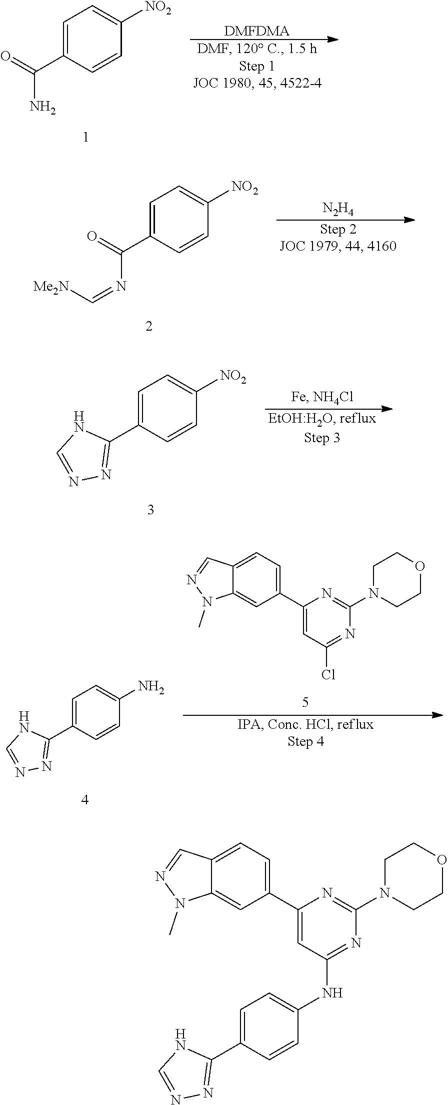

### Step 1: Synthesis of (Z)-N-((dimethylamino)methylene)-4-nitrobenzamide (2)

A stirred solution of 4-nitrobenzamide 1 (1 g, 1 eq), DMFDMA (3 mL) in DMF (1 mL) was heated at 120° C. for 1.5 h. The progress of the reaction was monitored by TLC. After completion of the reaction, the reaction mixture was quenched with ice water and filtered. The residue was washed with water and dried under vacuum to afford the title compound 2. LCMS (m/z): 222.00 (M+1).

### Step 2: Synthesis of 3-(4-nitrophenyl)-4H-1,2,4-triazole (3)

To a stirred solution of compound 2 (2.8 g, 1 eq) in acetic acid (10 mL), hydrazine hydrate (0.3 mL, 1.1 eq) was added and stirred at 90° C. for 1.5 h. The progress of the reaction was monitored by TLC. After completion of the reaction, the reaction mixture was evaporated under reduced pressure. The residue was neutralized with saturated sodium bicarbonate solution and extracted with ethyl acetate (3×50 mL). Combined organic extracts were washed with brine dried over anhydrous sodium sulfate and evaporated under reduced pressure to afford the title compound 3. LCMS (m/z): 190.95 (M+1).

### Step 3: Synthesis of 4-(4H-1,2,4-triazol-3-yl)aniline (4)

To a stirred solution of compound 3 (0.05 g, 1 eq) in ethanol (5 mL), iron powder (0.074 g, 5 eq), water (3 mL) and ammonium chloride (0.069 g, 5 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (50 mL) and washed with brine. Combined organic extracts were dried over anhydrous sodium sulfate and evaporated under reduced pressure to afford the title compound 4. LCMS (m/z): 161.00 (M+1).

### Step 4: Synthesis of N-(4-(4H-1,2,4-triazol-3-yl)phenyl)-6-(1-methyl-1H-indazol-6-yl)-2-morpholino pyrimidin-4-amine

The title compound has been synthesized by following the general procedure described above for displacement reaction (IPA, Conc. HCl) by using chloro compound 5 and 4-(4H-1,2,4-triazol-3-yl)aniline 4. ¹ NMR (400 MHz, DMSO-d₆) δ: 10.28 (s, 1H), 8.57 (s, 1H), 8.24 (s, 1H), 8.15 (s, 1H), 8.07-8.00 (m, 2H), 7.94-7.82 (m, 3H), 7.69 (d, J=8.3 Hz, 1H), 6.79 (s, 1H), 4.15 (s, 3H), 3.86-3.76 (m, 8H); HPLC purity: 99.85%; LCMS Calculated for C₂₄H₂₃N₉O: 453.20; Observed: 454.35 (M+1).

### Example 44

### Synthesis of 6-(1-methyl-1H-indazol-6-yl)-N-(4-(5-methyl-4H-1,2,4-triazol-3-yl)phenyl)-2-morpholino pyrimidin-4-amine

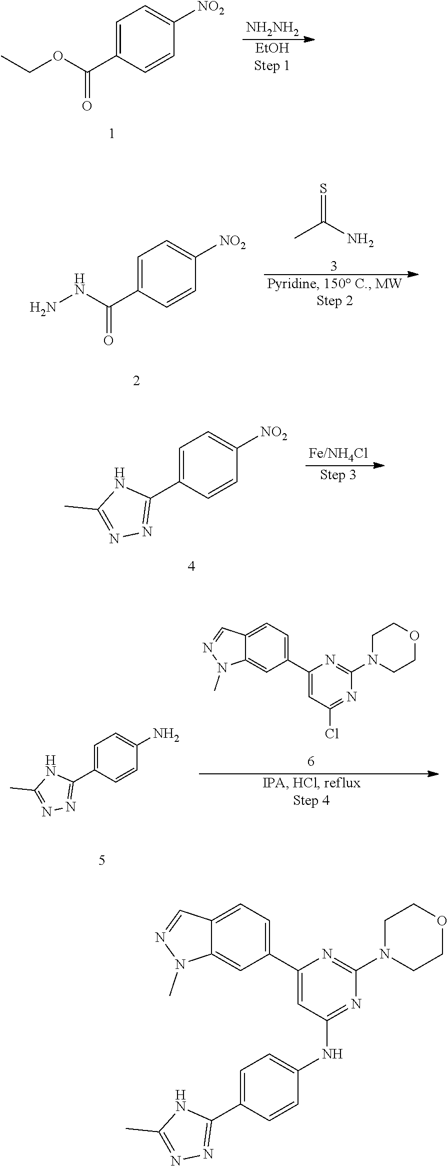

### Step 1: Synthesis of 4-nitrobenzohydrazide (2)

To a stirred solution of compound 1 (2 g, 1 eq) in ethanol (20 mL), hydrazine monohydrate (1.54 mL, 3 eq) was added and heated to reflux for overnight. The progress of the reaction was monitored by TLC. After completion of the reaction, the reaction mixture was filtered and residue was washed with ethanol and dried under vacuum to afford title compound 2. ¹H NMR (400 MHz, DMSO-d₆) δ: 10.09 (s, 1H), 8.26 (d, J=8.8 Hz, 2H), 8.01 (d, J=9.2 Hz, 2H), 4.60 (s, 2H).

### Step 2: Synthesis of 3-methyl-5-(4-nitrophenyl)-4H-1,2,4-triazole (4)

To a stirred solution of compound 2 (0.5 g, 1 eq) in pyridine (5 mL), thioacetamide 3 (0.31 g, 1.5 eq) was added and heated in microwave at 150° C. for 1 h. The progress of the reaction was monitored by TLC. After completion of the reaction, the reaction mixture was quenched with 1 N HCl and filtered. The residue was washed with diethyl ether to afford the title compound 4. LCMS (m/z): 205.15 (M+1).

### Step 3: Synthesis of 4-(5-methyl-4H-1,2,4-triazol-3-yl)aniline(5)

To a stirred solution of compound 4 (0.3 g, 1 eq), in ethanol: water(1:1; 20 mL), Fe powder (0.288 g, 3.5 eq) and ammonium chloride (0.272 g,3.5 eq) were added and heated to reflux for 1 h. The progress of the reaction was monitored by TLC. After completion of the reaction, the reaction mixture was filtered through celite and evaporated to dryness. The residue was diluted with water and extracted with ethyl acetate (3×25 mL). Combined organic extracts were dried over anhydrous sodium sulfate and evaporated under reduced pressure to afford the title compound 5. LCMS (m/z): 175.00 (M+1).

### Step 4: Synthesis of 6-(1-methyl-1H-indazol-6-yl)-N-(4-(5-methyl-4H-1,2,4-triazol-3-yl)phenyl)-2-morpholinopyrimidin-4-amine

To a stirred solution of compound 6 (0.2 g, 1 eq), compound 5 (0.158 g, 1.5 eq) in IPA (2 mL), Conc. HCl (1 mL) was added and heated to reflux for 18 h. The progress of the reaction was monitored by TLC. After completion of the reaction, the reaction mixture was quenched with aqueous sodium hydrogen carbonate (saturated) solution and extracted with ethyl acetate (3×25 mL).Combined organic extracts were washed with water, brine, dried over anhydrous sodium sulfate and evaporated under reduced pressure. The crude product was purified by column chromatography on silica gel 100-200 mesh using 80% EtOAc-hexane to enrich the purity and further purified by preparative HPLC to afford the desired product. ¹H NMR (400 MHz, DMSO-d₆) δ: 10.14 (s, 1H), 8.24 (s, 1H), 8.13 (s, 1H), 8.01 (d, J=8.3 Hz, 2H), 7.87 (d, J=7.9 Hz, 3H), 7.72 (d, J=8.5 Hz, 1H), 6.80 (s, 1H), 4.14 (s, 3H), 3.84-3.75 (m, 8H), 2.33 (s, 3H); HPLC purity: 99.6%; LCMS Calculated for C₂₅H₂₅N₉O (free base): 467.22; observed: 468.35 (M+1).

### Examples 45-52

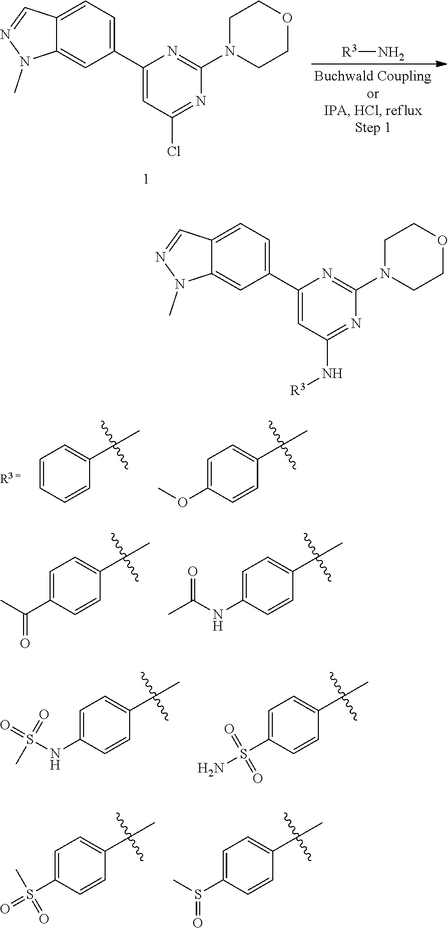

The following compounds were prepared using the above procedure.

6-(1-methyl-1H-indazol-6-yl)-2-morpholino-N-phenylpyrimidin-4-amine: The title compound has been synthesized by the general procedure described above (IPA, Conc. HCl) by using compound 1 and aniline. ¹H NMR (400 MHz, DMSO-d₆) δ: 8.22 (s, 1H), 8.15 (s, 1H), 7.91 (d, J=8.4 Hz, 1H), 7.67 (dd, J=24.2, 8.0 Hz, 3H), 7.38 (t, J=7.8 Hz, 2H), 7.08 (t, J=7.3 Hz, 1H), 6.74 (s, 1H), 4.14 (s, 3H), 3.84-3.74 (m, 8H); HPLC purity: 98.68%; LCMS Calculated for C₂₂H₂₂N₆O (free base): 386.19; Observed: 387.25 (M+1).

N-(4-methoxyphenyl)-6-(1-methyl-1H-indazol-6-yl)-2-morpholinopyrimidin-4-amine: The title compound has been synthesized by following the general procedure described above for displacement reaction (IPA, Conc. HCl) by using chloro compound 1 and 4-methoxyaniline. ¹H NMR (400 MHz, DMSO-d₆) δ: 8.17 (d, J=19.4 Hz, 2H), 7.91 (d, J=8.4 Hz, 1H), 7.58 (d, J=8.5 Hz, 3H), 6.97 (d, J=8.4 Hz, 2H), 6.64 (s, 1H), 4.14 (s, 3H), 3.85-3.70 (m, 11H); HPLC purity: 95.75%; LCMS Calculated for C₂₃H₂₄N₆O₂ (free base): 416.20; Observed: 417.30 (M+1).

1-(4-((6-(1-methyl-1H-indazol-6-yl)-2-morpholinopyrimidin-4-yl)amino)phenyl)ethan-1-one: The title compound has been synthesized by following the general procedure described above for displacement reaction (IPA, Conc. HCl) by using chloro compound 1 and 1-(4-aminophenyl)ethan-1-one. ¹H NMR (400 MHz, DMSO-d₆) δ: 10.02 (s, 1H), 8.24 (d, J=1.4 Hz, 1H), 8.11 (d, J=0.9 Hz, 1H), 8.00-7.92 (m, 2H), 7.91-7.82 (m, 3H), 7.75 (dd, J=8.5, 1.4 Hz, 1H), 6.80 (s, 1H), 4.14 (s, 3H), 3.84-3.75 (m, 8H), 2.53 (s, 3H); HPLC purity: 95.51%; LCMS Calculated for C₂₄H₂₄N₆O₂ (free base): 428.20; Observed: 429.00 (M+1).

N-(4-((6-(1-methyl-1H-indazol-6-yl)-2-morpholinopyrimidin-4-yl)amino)phenyl)acetamide: The title compound has been synthesized by following the general procedure described above for displacement reaction (IPA, Conc. HCl) by using chloro compound 1 and N-(4-aminophenyl) acetamide. ¹H NMR (400 MHz, DMSO-d₆) δ: 10.57 (s, 1H), 10.06 (s, 1H), 8.25-8.14 (m, 2H), 7.93 (d, J=8.4 Hz, 1H), 7.62-7.54 (m, 5H), 6.73 (s, 1H), 4.14 (s, 3H), 3.83-3.75 (m, 8H), 2.05 (s, 3H); HPLC purity: 95.12%; LCMS Calculated for C₂₄H₂₅N₇O₂ (free base): 443.21; Observed: 444.40 (M+1).

N-(4-((6-(1-methyl-1H-indazol-6-yl)-2-morpholinopyrimidin-4-yl) amino) phenyl) methane sulfonamide: The title compound has been synthesized by following the general procedure described above for displacement reaction (IPA, Conc. HCl) by using chloro compound 1 and N-(4-aminophenyl) methanesulfonamide. ¹H NMR (400 MHz, DMSO-d₆) δ: 9.49 (s, 1H), 9.43 (s, 1H), 8.22 (s, 1H), 8.09 (s, 1H), 7.84 (d, J=8.5 Hz, 1H), 7.75 (dd, J=8.5, 1.4 Hz, 1H), 7.70-7.62 (m, 2H), 7.23-7.14 (m, 2H), 6.69 (s, 1H), 4.12 (s, 3H), 3.82-3.72 (m, 8H), 2.94 (s, 3H); HPLC purity: 98.54%; LCMS Calculated for C₂₃H₂₅N₇O₃S (free base): 479.17; Observed: 480.25 (M+1).

4-((6-(1-methyl-1H-indazol-6-yl)-2-morpholinopyrimidin-4-yl)amino)benzenesulfonamide: The title compound has been synthesized by following the general procedure described above for displacement reaction (IPA, Conc. HCl) by using chloro compound 1 and 4-aminobenzenesulfonamide. ¹H NMR (400 MHz, DMSO-d₆) δ: 10.42 (s, 1H), 8.24 (s, 1H), 8.13 (s, 1H), 7.93-7.86 (m, 3H), 7.81 (d, J=8.5 Hz, 2H), 7.69 (d, J=8.4 Hz, 1H), 7.24 (s, 2H), 6.84 (s, 1H), 4.14 (s, 3H), 3.88-3.75 (m, 8H); HPLC purity: 99.61%; LCMS Calculated for C₂₂H₂₃N₇O₃S (free base): 465.16; Observed: 466.25 (M+1).

6-(1-methyl-1H-indazol-6-yl)-N-(4-(methylsulfonyl)phenyl)-2-morpholinopyrimidin-4-amine: The title compound has been synthesized by following the general procedure described above for displacement reaction (IPA, Conc. HCl) by using chloro compound 1 and 4-(methylsulfonyl)aniline. ¹H NMR (400 MHz, DMSO-d₆) δ: 10.27 (s, 1H), 8.25 (s, 1H), 8.12 (s, 1H), 7.98 (d, J=8.6 Hz, 2H), 7.88 (d, J=8.4 Hz, 3H), 7.74 (d, J=8.5 Hz, 1H), 6.83 (s, 1H), 4.14 (s, 3H), 3.84-3.75 (m, 8H), 3.17 (s, 3H); HPLC purity: 98.67%; LCMS Calculated for C₂₃H₂₄N₆O₃S (free base): 464.16; Observed: 465.30 (M+1).

6-(1-methyl-1H-indazol-6-yl)-N-(4-(methylsulfinyl)phenyl)-2-morpholinopyrimidin-4-amine:The title compound has been synthesized by following the General procedure for Buchwald Coupling described above using chloro compound 1 and 4-(methylsulfinyl)aniline. ¹H NMR (400 MHz, DMSO-d₆) δ: 10.05 (s, 1H), 8.24 (d, J=1.3 Hz, 1H), 8.12 (d, J=1.0 Hz, 1H), 7.96-7.85 (m, 3H), 7.70 (dd, J=23.8, 8.6 Hz, 3H), 6.78 (s, 1H), 4.14 (s, 3H), 3.84-3.75 (m, 8H), 2.73 (s, 3H); HPLC purity: 98.54%; LCMS Calculated for C₂₃H₂₄N₆O₂S (free base): 448.17; Observed: 449.30 (M+1).

### Examples 53-54

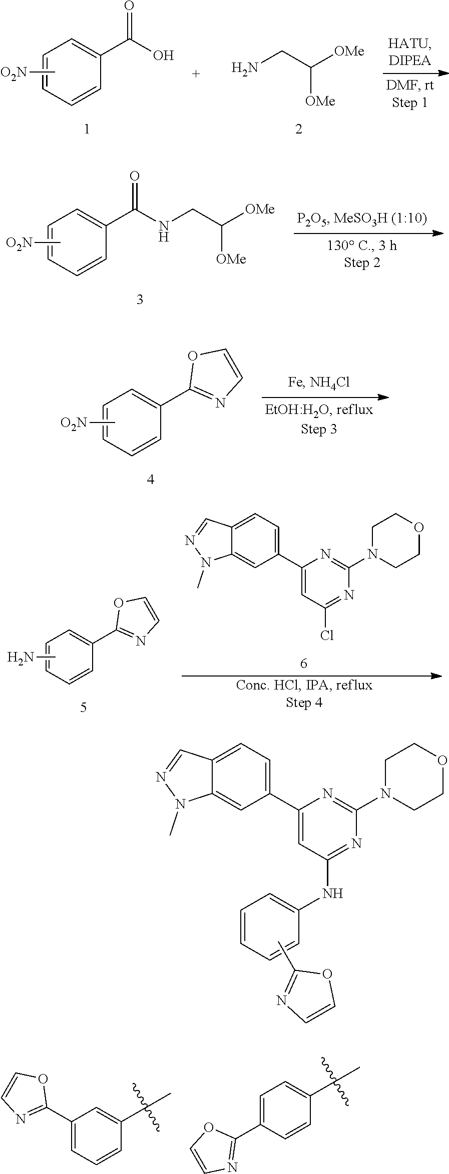

### Step 1: General Procedure for Amide Coupling

To a mixture of acid 1 (1 eq) and amine 2 (1.5 eq) in DMF, DIPEA (2 eq) was added and stirred at room temperature for 10 min. HATU (1.2 eq) was added slowly and stirred at room temperature for 2 h. The progress of the reaction was monitored by TLC. After completion of the reaction, water was added and extracted with ethyl acetate. Combined organic extracts were dried over anhydrous sodium sulfate and evaporated under reduced pressure. The crude product was purified by column chromatography to afford the desired product 3.

The intermediates listed in the following table were prepared in a similar manner starting with appropriate acid 1 and compound 2.

| Structure | LCMS |
|---|---|
| 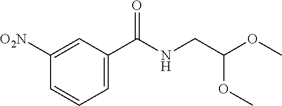 | LCMS (m/z): 208.90 (M + 1; aldehyde) |
| 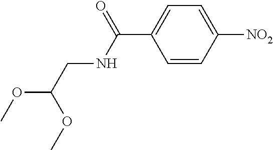 | LCMS (m/z): 209.05 (M + 1; aldehyde) |

### Step 2: General Procedure for Cyclization

To a stirred solution of compound 3 (1 eq) in P₂O₅ and methane sulfonic acid (1:10) was heated at 130° C. for 5 h. The progress of the reaction was monitored by TLC. After completion of the reaction, the reaction mixture was quenched with saturated sodium bicarbonate solution and extracted with ethyl acetate. Combined organic extracts were washed with brine, dried over anhydrous sodium sulfate and evaporated under reduced pressure to afford the title compound 4.

The intermediates 4 listed in the following table were prepared in a similar manner.

| Structure | LCMS |
|---|---|
| 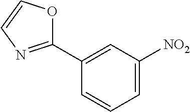 [SMILES: O=[N+]([O-])c1cccc(-c2ncco2)c1] | LCMS (m/z): 190.95 (M + 1) |
| 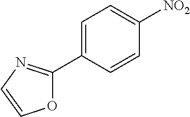 [SMILES: O=[N+]([O-])c1ccc(-c2ncco2)cc1] | LCMS (m/z): 190.95 (M + 1) |

### Step 3: General Procedure for Reduction

To a stirred solution of compound 4 (1 eq) in ethanol, iron powder (5 eq), water and ammonium chloride (5 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate and washed with brine, dried over anhydrous sodium sulfate and evaporated under reduced pressure to afford the title compound 5.

The intermediates listed in the following table were prepared in a similar manner starting with appropriate compound 4.

| Structure | LCMS |
|---|---|
| 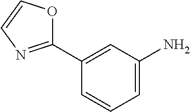 [SMILES: Nc1cccc(-c2ncco2)c1] | LCMS (m/z): 160.95 (M + 1) |
| 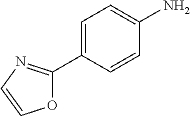 [SMILES: Nc1ccc(-c2ncco2)cc1] | LCMS (m/z): 160.90 (M + 1) |

6-(1-methyl-1H-indazol-6-yl)-2-morpholino-N-(3-(oxazol-2-yl)phenyl)pyrimidin-4-amine:The title compound has been synthesized by following the general procedure described above for displacement reaction (IPA, Conc. HCl) by using chloro compound 6 and corresponding amine 5. ¹H NMR (400 MHz, DMSO-d₆) δ: 10.24 (s, 1H), 8.88 (s, 1H), 8.25 (d, J=8.6 Hz, 2H), 8.13 (s, 1H), 7.90 (d, J=8.5 Hz, 1H), 7.74-7.59 (m, 3H), 7.50 (t, J=7.9 Hz, 1H), 7.41 (s, 1H), 6.77 (s, 1H), 4.14 (s, 3H), 3.91-3.78 (m, 8H); HPLC purity: 99.64%; LCMS Calculated for C₂₅H₂₃N₇O₂(free base): 453.19; Observed: 454.35 (M+1).

6-(1-methyl-1H-indazol-6-yl)-2-morpholino-N-(4-(oxazol-2-yl)phenyl)pyrimidin-4-amine: The title compound has been synthesized by following the general procedure described above for displacement reaction (IPA, Conc. HCl) by using chloro compound 6 and corresponding amine 5. ¹H NMR (400 MHz, DMSO-d₆) δ: 9.77 (s, 1H), 8.25 (s, 1H), 8.16 (s, 1H), 8.10 (s, 1H), 7.95 (d, J=8.5 Hz, 2H), 7.91-7.82 (m, 3H), 7.78 (d, J=8.6 Hz, 1H), 7.34 (s, 1H), 6.77 (s, 1H), 4.13 (s, 3H), 3.87-3.74 (m, 8H); HPLC purity: 99.29%; LCMS Calculated for C₂₅H₂₃N702:453.19; Observed: 454.30 (M+1).

### Example 55

### Synthesis of 6-(6-((4-chlorophenyl)amino)-2-morpholinopyrimidin-4-yl)-N,N,1-trimethyl-1H-indazol-3-amine

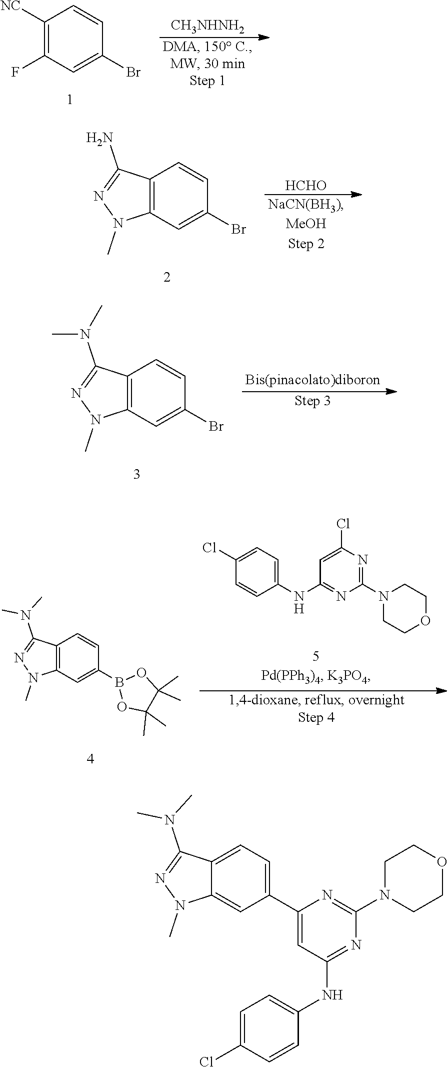

### Step 1: Synthesis of 6-bromo-1-methyl-1H-indazol-3-amine (2)

To a stirred solution of compound 1 (1 g, 1 eq) in DMA (3 mL), DIPEA (0.645 g, 1 eq) and methyl hydrazine (0.276 g, 1.2 eq) were added and heated at 150° C. for 30 min in microwave. The progress of the reaction was monitored by TLC. After completion of the reaction, the reaction mixture was diluted with ethyl acetate (50 mL), washed with water, dried over anhydrous sodium sulfate and evaporated under reduced pressure to afford the title compound 2. LCMS (m/z): 227.95 (M+2).

### Step 2: Synthesis of 6-bromo-N,N,1-trimethyl-1H-indazol-3-amine (3)

To a stirred solution of compound 2 (1 g, 1 eq) in methanol (15 mL), formaldehyde (1.66 mL, 5 eq) was added slowly at 0° C. and stirred at room temperature for 10 min. The reaction mixture was cooled to 0° C. and sodium cyanoborohydride (1.11 g, 4 eq) was added at same temperature. The reaction mixture was stirred at room temperature for 18 h. The progress of the reaction was monitored by TLC. After completion of the reaction, the reaction mixture was evaporated under reduced pressure. The residue was dissolved in ethyl acetate (100 mL), washed with brine, dried over anhydrous sodium sulfate and evaporated under reduced pressure to afford the title compound 3. LCMS (m/z): 256.00 (M+2).

### Step 3: Synthesis of N,N,1-trimethyl-6-(4,4,5,5-tetramethyl-1,3,2-dioxaborolan-2-yl)-1H-indazol-3-amine (4)

The title compound has been synthesized by following the General Procedure for Boronate Ester Preparation described above using bromo compound 3 and Bis (pinacolato)diboron. LCMS (m/z): 302.15 (M+1).

### Step 4: Synthesis of 6-(6-((4-chlorophenyl)amino)-2-morpholinopyrimidin-4-yl)-N,N,1-trimethyl-1H-indazol-3-amine

The title compound has been synthesized by following the General Procedure for Suzuki Coupling described above using chloro compound 5 and Boronate ester 4. ¹H NMR (400 MHz, DMSO-d₆) δ: 10.47 (s, 1H), 8.04-7.95 (m, 2H), 7.74 (d, J=8.4 Hz, 2H), 7.43 (d, J=8.4 Hz, 3H), 6.76 (s, 1H), 3.92 (s, 3H), 3.86-3.74 (m, 8H), 3.04 (s, 6H); HPLC purity: 98.86%; LCMS Calculated for C₂₄H₂₆ClN₇O (free base): 463.19; Observed: 464.25 (M+1).

### Examples 56-57

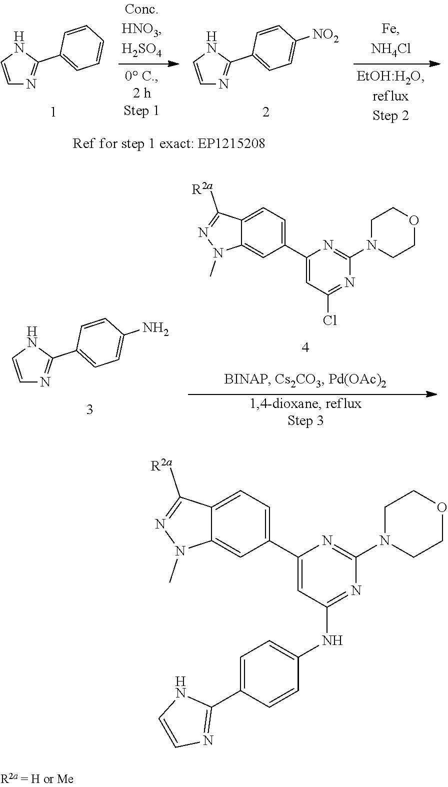

### Step 1: Synthesis of 2-(4-nitrophenyl)-1H-imidazole (2)

To a stirred solution of compound 1 (2 g, 1 eq) in Conc. H₂SO₄ (8 mL), nitrating mixture (0.88 mL Conc. HNO₃+2 mL Conc. H₂SO₄) was added at 0° C. and stirred at same temperature for 2 h. The progress of the reaction was monitored by TLC. After completion of the reaction, the reaction mixture was poured onto ice water and basified to pH 9 using 2N NaOH. The precipitated solid was filtered, washed with water and dried under reduced pressure to afford the title compound 2. LCMS (m/z): 190.00 (M+1).

### Step 2: Synthesis of 4-(1H-imidazol-2-yl) aniline (3)

To a stirred solution of compound 2 (0.5 g, 1 eq) in ethanol (12 mL), iron powder (0.5 g), water (6 mL) and ammonium chloride (0.5 g) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (100 mL), washed with brine, dried over anhydrous sodium sulfate and evaporated under reduced pressure to afford the title compound 3. LCMS (m/z): 160.05 (M+1).

### Step 3

N-(4-(1H-imidazol-2-yl)phenyl)-6-(1-methyl-1H-indazol-6-yl)-2-morpholino pyrimidin-4-amine: The title compound has been synthesized by following the General procedure for Buchwald Coupling described above using corresponding chloro compound 4 and amine compound 3. ¹H NMR (400 MHz, MeOD) δ: 8.15 (d, J=5.9 Hz, 2H), 8.00 (d, J=6.9 Hz, 5H), 7.68 (s, 2H), 7.55 (dd, J=8.4, 1.4 Hz, 1H), 6.72 (s, 1H), 4.20 (s, 3H), 3.99-3.84 (m, 8H); HPLC purity: 97.85%; LCMS Calculated for C₂₅H₂₄N₈₀ (free base): 452.21; Observed: 453.40 (M+1).

N-(4-(1H-imidazol-2-yl)phenyl)-6-(1,3-dimethyl-1H-indazol-6-yl)-2-morpholino pyrimidin-4-amine:The title compound has been synthesized by following the General procedure for Buchwald Coupling described above using corresponding chloro compound 4 and amine compound 3. ¹H NMR (400 MHz, DMSO-d₆) δ: 12.35 (s, 1H), 9.56 (s, 1H), 8.16 (s, 1H), 7.92-7.85 (m, 2H), 7.83-7.68 (m, 4H), 7.10-7.02 (m, 2H), 6.73 (s, 1H), 4.04 (s, 3H), 3.83-3.74 (m, 8H), 2.57 (s, 3H); HPLC purity: 94.71%; LCMS Calculated for C₂₆H₂₆N₈O: 466.22; Observed: 467.40 (M+1).

### Example 58-59

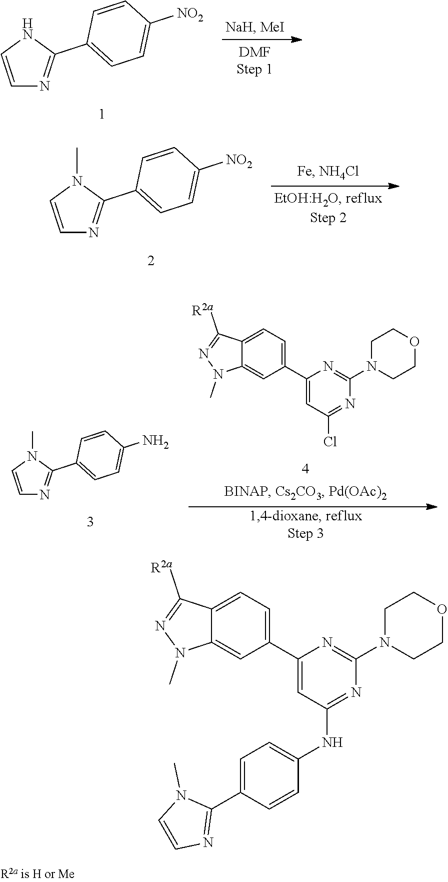

### Step 1: Synthesis of 1-methyl-2-(4-nitrophenyl)-1H-imidazole (2)

To a stirred solution of compound 1 (0.5 g, 1 eq) in DMF (10 mL), sodium hydride (0.095 g, 1.5 eq) was added at 0° C. and stirred for 15 min followed by the addition of methyl iodide (0.562 g, 1.5 eq) at 0° C. and stirred at room temperature for 30 min. The progress of the reaction was monitored by TLC. After completion of the reaction, the reaction mixture was quenched with ice water and extracted with ethyl acetate (2×25 mL). Combined organic extracts were washed with brine, dried over anhydrous sodium sulfate and evaporated under reduced pressure to afford the title compound 2. LCMS (m/z): 204.05 (M+1).

### Step 2: Synthesis of 4-(1-methyl-1H-imidazol-2-yl)aniline (3)

To a stirred solution of compound 2 (0.4 g, 1 eq) in ethanol (12 mL), iron powder (0.4 g), water (6 mL) and ammonium chloride (0.4 g) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (100 mL), washed with brine, dried over anhydrous sodium sulfate and evaporated under reduced pressure to afford the title compound 3. LCMS (m/z): 173.10 (M+).

### Step 3

N-(4-(1-methyl-1H-imidazol-2-yl)phenyl)-6-(1-methyl-1H-indazol-6-yl)-2-morpholinopyrimidin-4-amine:The title compound has been synthesized by following the General procedure for Buchwald Coupling described above using corresponding chloro compound 4 and amine compound 3. ¹H NMR (400 MHz, MeOD) δ: 8.15 (d, J=2.3 Hz, 2H), 8.03 (dd, J=20.5, 8.4 Hz, 3H), 7.91-7.83 (m, 2H), 7.68 (dd, J=13.8, 2.1 Hz, 2H), 7.55 (dd, J=8.4, 1.5 Hz, 1H), 6.75 (s, 1H), 4.20 (s, 3H), 4.01-3.85 (m, 11H); HPLC purity: 98.15%; LCMS Calculated for C₂₆H₂₆N₈O (free base): 466.22; Observed: 467.45 (M+1).

6-(1,3-dimethyl-1H-indazol-6-yl)-N-(4-(1-methyl-1H-imidazol-2-yl)phenyl)-2-morpholinopyrimidin-4-amine: The title compound has been synthesized by following the General procedure for Buchwald Coupling described above using corresponding chloro compound 4 and amine compound 3. ¹H NMR (400 MHz, DMSO-d₆, D₂O exchange) δ: 8.15 (s, 1H), 7.84-7.71 (m, 4H), 7.65-7.58 (m, 2H), 7.21 (d, J=1.3 Hz, 1H), 6.96 (d, J=1.3 Hz, 1H), 6.72 (s, 1H), 4.00 (s, 3H), 3.81-3.72 (m, 11H), 2.48 (s, 3H); HPLC purity: 98.77%; LCMS Calculated for C₂₇H₂₈N₈O: 480.24; Observed: 481.40 (M+1).

### Example 60

### Synthesis of N-(4-(1H-imidazol-4-yl)phenyl)-6-(1-methyl-1H-indazol-6-yl)-2-morpholinopyrimidin-4-amine

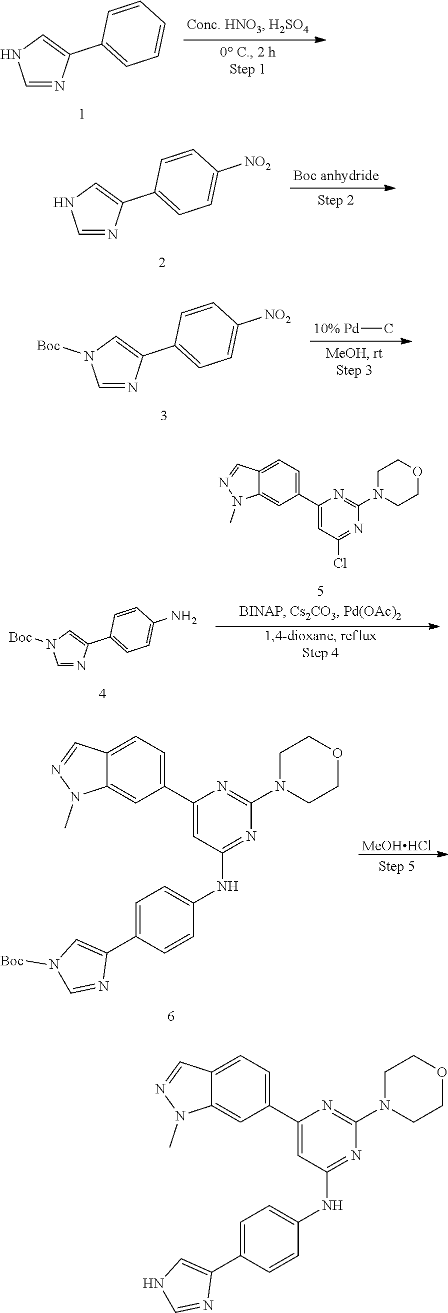

### Step 1: Synthesis of 4-(4-nitrophenyl)-1H-imidazole (2)

To a stirred solution of compound 1 (1 g, 1 eq) in Conc. H₂SO₄ (4 mL), nitrating mixture (0.44 mL Conc. HNO₃+1 mL Conc. H₂SO₄) was added at 0° C. and stirred at same temperature for 2 h. The progress of the reaction was monitored by TLC. After completion of the reaction, the reaction mixture was poured onto ice water and basified to pH 9 using 2N NaOH. The precipitated solid was filtered, washed with water and dried under reduced pressure to afford the title compound 2. LCMS (m/z): 190.00 (M+1).

### Step 2: Synthesis of tert-butyl 4-(4-nitrophenyl)-1H-imidazole-1-carboxylate (3)

To a stirred solution of NaH (0.069 g, 1.2 eq) in dry THF (10 mL), compound 2 (0.45 g, 1 eq) was added at 0° C. and stirred for 30 min. followed by the addition of Boc anhydride (0.57 g, 1.1 eq) at 0° C. The reaction mixture was stirred at room temperature for 2 h. The progress of the reaction was monitored by TLC. After completion of the reaction, the reaction mixture was diluted with water and extracted with ethyl acetate (3×25 mL). Combined organic extracts were washed with brine, dried over anhydrous sodium sulfate and evaporated under reduced pressure to afford title compound 3.

### Step 3: Synthesis of tert-butyl 4-(4-aminophenyl)-1H-imidazole-1-carboxylate (4)

To a stirred solution of compound 3 (0.4 g, 1 eq) in methanol (5 mL), 10% Pd/C (0.04 g) was added and stirred under hydrogen atmosphere (balloon pressure) at room temperature for 8 h. The progress of the reaction was monitored by TLC. After completion of the reaction, the reaction mixture was filtered through celite. The filtrate was evaporated under reduced pressure to afford title compound 4.

### Step 4: tert-butyl 4-(4-((6-(1-methyl-1H-indazol-6-yl)-2-morpholinopyrimidin-4-yl)amino)phenyl)-1H-imidazole-1-carboxylate (6)

The title compound has been synthesized by following the General procedure for Buchwald Coupling described above using chloro compound 5 and amine compound 4. LCMS (m/z): 553.40 (M+1).

### Step 5: Synthesis of N-(4-(1H-imidazol-4-yl)phenyl)-6-(1-methyl-1H-indazol-6-yl)-2-morpholino pyrimidin-4-amine

To a stirred solution of compound 6 (0.06 g, 1 eq) in methanol (5 mL), methanolic HCl (3 mL) was added and stirred at room temperature for 1 h. The progress of the reaction was monitored by TLC. After completion of the reaction, the reaction mixture was evaporated to dryness. The crude product was purified by washing with diethyl ether to afford title compound as HCl salt. HPLC purity: 99.04%; LCMS Calculated for C₂₅H₂₄N₈₀ (free base): 452.21; Observed: 453.30 (M+1).

### Examples 61-62

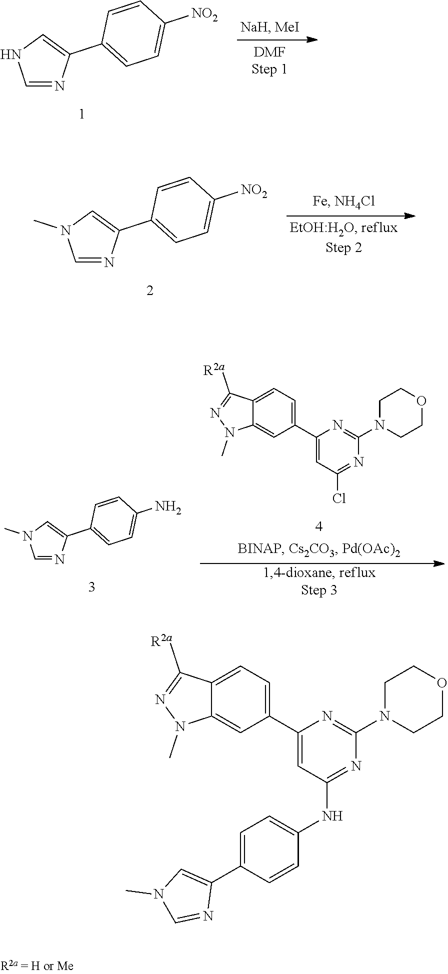

Step 1: Synthesis of 1-methyl-4-(4-nitrophenyl)-1H-imidazole (2)

To a stirred solution of compound 1 (1 g, 1 eq) in DMF (15 mL), sodium hydride(0.315 g, 1.5 eq) was added at 0° C. and stirred for 15 min followed by the addition of methyl iodide (1.12 g, 1.5 eq) at 0° C. and stirred at room temperature for 30 min. The progress of the reaction was monitored by TLC. After completion of the reaction, the reaction mixture was quenched with ice water and extracted with ethyl acetate (2×25 mL). Combined organic extracts were washed with brine, dried over anhydrous sodium sulfate and evaporated under reduced pressure to afford the title compound 2. LCMS (m/z): 204.10 (M+1).

### Step 2: Synthesis of 4-(1-methyl-1H-imidazol-4-yl)aniline (3)

To a stirred solution of compound 2 (0.7 g, 1 eq) in ethanol (10 mL), iron powder (0.731 g, 4 eq), water (5 mL) and ammonium chloride (0.731 g, 4 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in 20% methanol in dichloromethane, filtered through celite and evaporated under reduced pressure to afford the title compound 3. LCMS (m/z): 174.05 (M+1).

N-(4-(1-methyl-1H-imidazol-4-yl)phenyl)-6-(1-methyl-1H-indazol-6-yl)-2-morpholinopyrimidin-4-amine: The title compound has been synthesized by following the General procedure for Buchwald Coupling described above using corresponding chloro compound 4 and amine compound 3. ¹H NMR (400 MHz, DMSO-d₆) δ: 9.43 (s, 1H), 8.23 (s, 1H), 8.09 (s, 1H), 7.84 (d, J=8.4 Hz, 1H), 7.80-7.63 (m, 5H), 7.60 (s, 1H), 7.50 (s, 1H), 6.71 (s, 1H), 4.13 (s, 3H), 3.82-3.79 (m, 8H), 3.67 (s, 3H); HPLC purity: 97.07%; LCMS Calculated for C₂₆H₂₆N80: 466.22; Observed: 467.35 (M+1).

Synthesis of 6-(1,3-dimethyl-1H-indazol-6-yl)-N-(4-(1-methyl-1H-imidazol-4-yl)phenyl)-2-morpholinopyrimidin-4-amine: The title compound has been synthesized by following the General procedure for Buchwald Coupling described above using corresponding chloro compound 4 and amine compound 3. Structure has been confirmed by NOE. ¹H NMR (400 MHz, MeOD) δ: 9.01 (s, 1H), 8.05 (s, 1H), 7.97-7.77 (m, 6H), 7.50 (d, J=8.4 Hz, 1H), 6.68 (s, 1H), 4.11 (s, 3H), 4.01 (s, 3H), 3.97-3.83 (m, 8H), 2.60 (s, 3H); HPLC purity: 98.4%; LCMS Calculated for C₂₇H₂₈N₈₀ (free base): 480.24; Observed: 481.40 (M+1).

### Example 63

### Synthesis of N-(6-(1-methyl-1H-indazol-6-yl)-2-morpholinopyrimidin-4-yl)-1H-indazol-5-amine

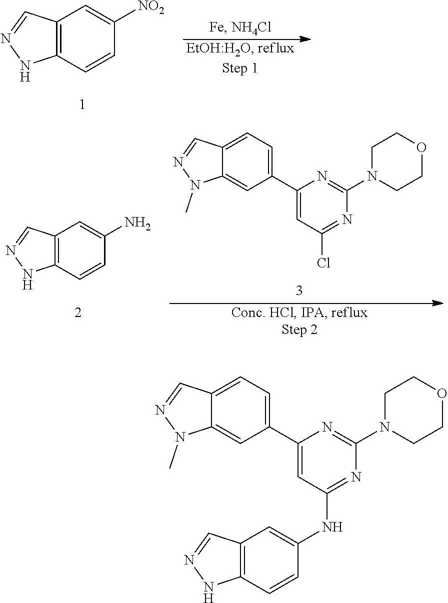

### Step 1: Synthesis of 1H-indazol-5-amine (2)

To a stirred solution of compound 1 (0.5 g, 1 eq) in ethanol (10 mL), iron powder (0.65 g, 4 eq), water (5 mL) and ammonium chloride (0.65 g. 4 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (50 mL), washed with brine, dried over anhydrous sodium sulfate and evaporated under reduced pressure to afford the title compound 2. LCMS (m/z): 133.95 (M+1).

### Step 2: Synthesis of N-(6-(1-methyl-1H-indazol-6-yl)-2-morpholinopyrimidin-4-yl)-1H-indazol-5-amine

The title compound has been synthesized by following the general procedure described above for displacement reaction (IPA, Conc. HCl) by using chloro compound 3 and amino compound 2. ¹H NMR (400 MHz, DMSO-d₆) δ: 12.96 (s, 1H), 9.37 (s, 1H), 8.22 (s, 1H), 8.12-8.00 (m, 3H), 7.83 (d, J=8.5 Hz, 1H), 7.75 (d, J=8.5 Hz, 1H), 7.60 (s, 2H), 6.68 (s, 1H), 4.12 (s, 3H), 3.82-3.72 (m, 8H); HPLC purity: 99.65%; LCMS Calculated for C₂₃H₂₂N₈O: 426.19; Observed: 427.25 (M+1).

### Examples 64-65

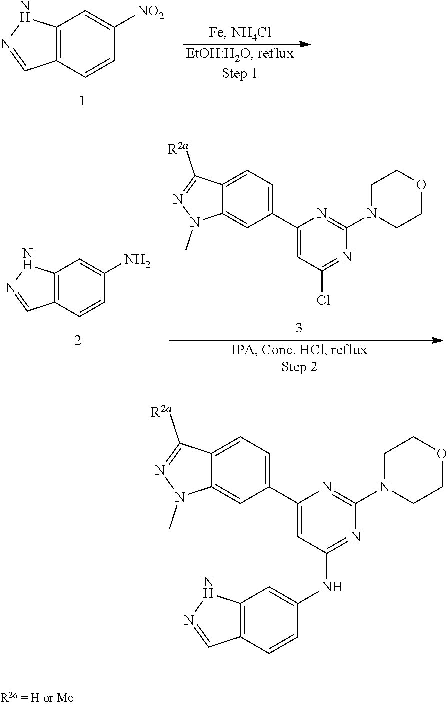

### Step 1: Synthesis of 1H-indazol-6-amine (2)

To a stirred solution of compound 1 (2 g, 1 eq) in ethanol (20 mL), iron powder (3.4 g, 5 eq), water (10 mL) and ammonium chloride (3.18 g. 5 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (50 mL), washed with brine, dried over anhydrous sodium sulfate and evaporated under reduced pressure to afford the title compound 2. LCMS (m/z): 134.05 (M+1).

### Step 2

N-(6-(1-methyl-1H-indazol-6-yl)-2-morpholinopyrimidin-4-yl)-1H-indazol-6-amine: The title compound has been synthesized by following the general procedure described above for displacement reaction (IPA, Conc. HCl) by using corresponding chloro compound 3 and amine 2. 41 NMR (400 MHz, DMSO-d₆) δ: 10.97 (s, 1H), 8.27-8.13 (m, 3H), 8.04 (s, 1H), 7.94 (d, J=8.4 Hz, 1H), 7.74 (d, J=8.6 Hz, 1H), 7.57 (d, J=8.5 Hz, 1H), 7.31 (d, J=8.8 Hz, 1H), 6.90 (s, 1H), 4.15 (s, 3H), 3.83-3.75 (m, 8H); HPLC purity: 99.44%; LCMS Calculated for C₂₃H₂₂N₈O (free base): 426.19; Observed: 427.35 (M+1).

N-(6-(1,3-dimethyl-1H-indazol-6-yl)-2-morpholinopyrimidin-4-yl)-1H-indazol-6-amine: The title compound has been synthesized by following the general procedure described above for displacement reaction (IPA, Conc. HCl) by using corresponding chloro compound 3 and amine 2. ¹H NMR (400 MHz, MeOD) δ: 8.24 (s, 1H), 8.08 (s, 1H), 8.00 (s, 1H), 7.83 (d, J=8.3 Hz, 1H), 7.74 (d, J=8.6 Hz, 1H), 7.64 (d, J=8.5 Hz, 1H), 7.20 (d, J=8.5 Hz, 1H), 6.67 (s, 1H), 4.08 (s, 3H), 3.98-3.86 (m, 8H), 2.58 (s, 3H); HPLC purity: 97.87%; LCMS Calculated for C₂₄H₂₄N₈O: 440.21; Observed: 441.40 (M+1).

### Examples 66-67

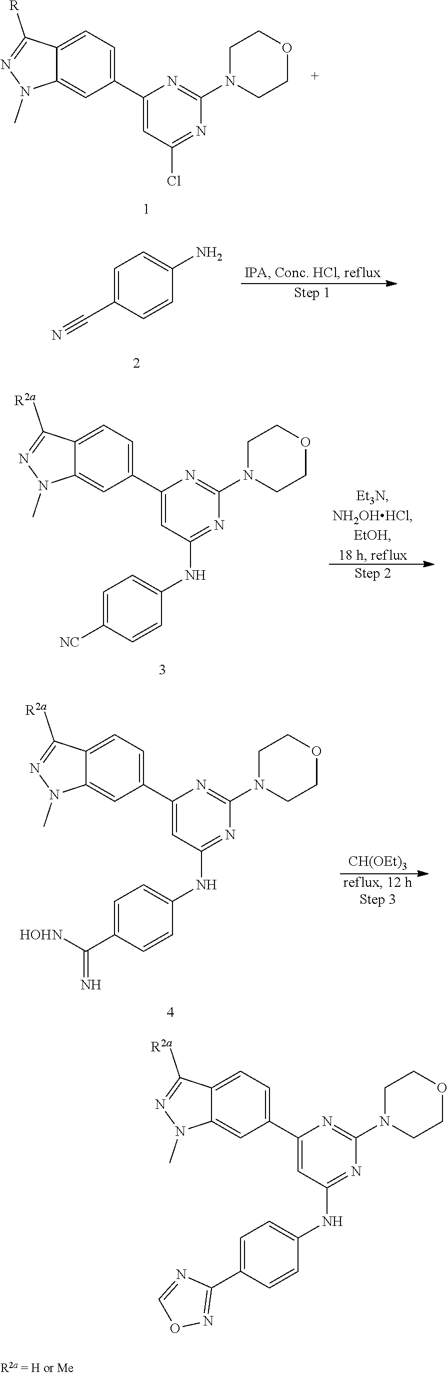

### Step 1: Synthesis of 4-((6-(1-methyl-1H-indazol-6-yl)-2-morpholinopyrimidin-4-yl)amino)benzonitrile (3)

The title compound has been synthesized by following the general procedure described above for displacement reaction (IPA, Conc. HCl) by using corresponding chloro compound 1 and 4-cyano aniline 2. LCMS (m/z): 412.30 (M+1).

### Step 1: Synthesis of 4-46-(1,3-dimethyl-1H-indazol-6-yl)-2-morpholinopyrimidin-4-yl)amino) benzonitrile (3)

The title compound has been synthesized by following the general procedure described above for displacement reaction (IPA, Conc. HCl) by using corresponding chloro compound 1 and 4-cyano aniline 2. LCMS (m/z): 426.25 (M+1).

### Step 2: Synthesis of N-hydroxy-4-((6-(1-methyl-1H-indazol-6-yl)-2-morpholinopyrimidin-4-yl)amino) benzimidamide

To a stirred solution of corresponding compound 3 (0.1 g, 1 eq) in ethanol (1 mL), hydroxyl amine hydrochloride (0.037g, 2.2 eq) and triethylamine (0.056 g, 2.3 eq) were added at room temperature. The reaction mixture was heated to reflux for 18 h. The progress of the reaction was monitored by TLC. After completion of the reaction, the reaction mixture was evaporated under reduced pressure. The residue was dissolved in water and extracted with ethyl acetate (2×25 mL). Combined organic extracts were dried over anhydrous sodium sulfate and evaporated under reduced pressure. The crude product was purified by recrystallization using chloroform and hexane to afford the title compound. ¹H NMR (400 MHz, DMSO-d₆) δ: 9.56 (s, 1H), 9.47 (s, 1H), 8.24 (s, 1H), 8.10 (s, 1H), 7.85 (d, J=8.4 Hz, 1H), 7.80-7.60 (m, 5H), 6.74 (s, 1H), 5.72 (s, 2H), 4.13 (s, 3H), 3.86-3.70 (m, 8H); HPLC purity: 96.57%; LCMS Calculated for C₂₃H₂₄N₈O₂: 444.20; Observed: 445.25 (M+1).

### Synthesis of 4-((6-(1,3-dimethyl-1H-indazol-6-yl)-2-morpholinopyrimidin-4-yl)amino)-N-hydroxybenzimidamide (4)

The title compound has been synthesized by following the above procedure. LCMS (m/z): 459.0 (M+1).

### Step 3

N-(4-(1,2,4-oxadiazol-3-yl)phenyl)-6-(1-methyl-1H-indazol-6-yl)-2-morpholino pyrimidin-4-amine: A stirred solution of corresponding compound 4 (0.4 g, 1 eq) in triethyl orthoformate (10 mL) was heated at 150° C. for 15 h. The progress of the reaction was monitored by TLC. After completion of the reaction, the reaction mixture was evaporated under reduced pressure. The residue was dissolved in ethyl acetate and washed with water. The organic extracts were dried over anhydrous sodium sulfate and evaporated under reduced pressure. The crude product was purified by column chromatography on silica gel 100-200 mesh using 5% methanol in dichloromethane to afford the title compound. ¹H NMR (400 MHz, DMSO-d₆) δ: 9.85 (s, 1H), 9.65 (d, J=3.0 Hz, 1H), 8.25 (s, 1H), 8.11 (d, J=2.9 Hz, 1H), 8.02 (dd, J=8.8, 3.0 Hz, 2H), 7.89 (ddd, J=22.2, 8.6, 2.9 Hz, 3H), 7.82-7.74 (m, 1H), 6.78 (d, J=2.9 Hz, 1H), 4.14 (d, J=3.1 Hz, 3H), 3.88-3.71 (m, 8H); HPLC purity: 93.32%; LCMS Calculated for C₂₄H₂₂N₈O₂: 454.19; Observed: 455.30 (M+1).

N-(4-(1,2,4-oxadiazol-3-yl)phenyl)-6-(1,3-dimethyl-1H-indazol-6-yl)-2-morpholinopyrimidin-4-amine: The title compound has been synthesized by following the above procedure. ¹H NMR (400 MHz, DMSO-d₆) δ: 10.11 (s, 1H), 9.66 (s, 1H), 8.17 (s, 1H), 8.03 (d, J=8.4 Hz, 2H), 7.92 (d, J=8.4 Hz, 2H), 7.82 (d, J=8.4 Hz, 1H), 7.68 (d, J=8.4 Hz, 1H), 6.79 (s, 1H), 4.05 (s, 3H), 3.84-3.75 (m, 8H), 2.58 (s, 3H); HPLC purity: 96.64%; LCMS Calculated for C₂₅H₂₄N₈O₂ (free base): 468.20; Observed: 469.30 (M+1).

### Example 68

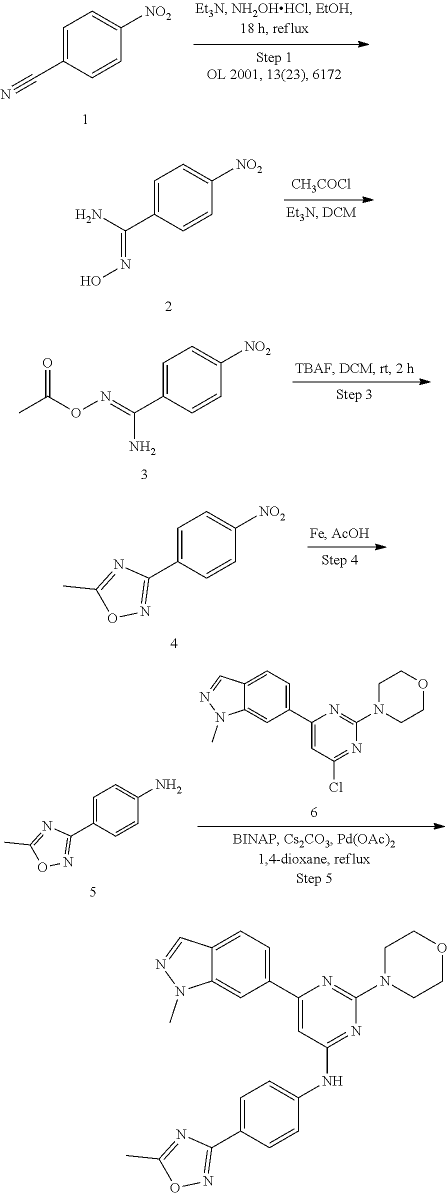

### Step 1: Synthesis of (Z)-N’-hydroxy-4-nitrobenzimidamide (2)

To a stirred solution of compound 1 (0.5 g, 1 eq) in ethanol (10 mL), hydroxyl amine hydrochloride (0.516 g, 2.2 eq) was added followed by the addition of triethylamine (1 mL, 2.3 eq). The reaction mixture was heated to reflux for 18 h. The progress of the reaction was monitored by TLC. After completion of the reaction, the reaction mixture was evaporated to dryness. The residue was diluted with water and extracted with ethyl acetate (2×25 mL). Combined organic extracts were dried over anhydrous sodium sulfate and evaporated under reduced pressure. The crude product was crystallized using chloroform and hexane to afford the title compound 2. LCMS (m/z): 181.90 (M+1).

### Step 2: Synthesis of (Z)-N′-acetoxy-4-nitrobenzimidamide (3)

To a stirred solution of compound 2 (0.2 g, 1 eq) in dichloromethane (10 mL), triethylamine (0.8 mL, 2.6 eq) was added followed by the addition of acetyl chloride (0.2 mL, 1.3 eq) at 0° C. and stirred for 1 h at same temperature. The reaction mixture was stirred at room temperature for 3 h. The progress of the reaction was monitored by TLC. After completion of the reaction, the reaction mixture was diluted with water and extracted with ethyl acetate (2×25 mL). Combined organic extracts were dried over anhydrous sodium sulfate and evaporated under reduced pressure. The crude product was recrystallized from ethanol to afford the title compound 3. LCMS (m/z): 224.05 (M+1).

### Step 3: Synthesis of 5-methyl-3-(4-nitrophenyl)-1,2,4-oxadiazole (4)

To a stirred solution of compound 3 (0.2 g, 1 eq) in dichloromethane (10 mL), tetrabutyl ammonium fluoride (1.3 mL, 3 eq) was added at 0° C. and stirred for 2 h at room temperature. The progress of the reaction was monitored by TLC. After completion of the reaction, the reaction mixture was diluted with ethyl acetate: diethyl ether (1:1) and washed with water. The organic extracts were washed with aq. potassium carbonate, brine, dried over anhydrous sodium sulfate and evaporated under reduced pressure. The crude product was washed with hexane to afford the title compound 4. ¹H NMR (400 MHz, CDC13) δ: 8.34 (d, J=9.2 Hz, 2H), 8.25 (d, J=9.2 Hz, 2H), 2.69 (s, 3H).

### Step 4: Synthesis of 4-(5-methyl-1,2,4-oxadiazol-3-yl)aniline (5)

To a stirred solution of compound 4 (0.28 g, 1 eq) in acetic acid (10 mL), iron powder (0.7 g, 9 eq) was added slowly. The reaction mixture was stirred at room temperature for 5 h. The progress of the reaction was monitored by TLC. After completion of the reaction, the reaction mixture was evaporated under reduced pressure. The residue was dissolved in water and ethyl acetate, filtered through celite and evaporated under reduced pressure. The residue was dissolved in ethyl acetate (50 mL), washed with brine, dried over anhydrous sodium sulfate and evaporated under reduced pressure to afford the title compound 2. LCMS (m/z): 176.00 (M+1).

### Step 5

N-(4-(5-methyl-1,2,4-oxadiazol-3-yl)phenyl)-6-(1-methyl4H-indazol-6-yl)-2-morpholinopyrimidin-4-amine: The title compound has been synthesized by following the General procedure for Buchwald Coupling described above using corresponding chloro compound 6 and amino compound 5. ¹H NMR (400 MHz, DMSO-d₆) δ: 10.09 (s, 1H), 8.25 (s, 1H), 8.12 (s, 1H), 7.98 (d, J=8.8 Hz, 2H), 7.94-7.83 (m, 3H), 7.73 (d, J=8.6 Hz, 1H), 6.79 (s, 1H), 4.14 (s, 3H), 3.88-3.75 (m, 8H), 2.65 (s, 3H); HPLC purity: 96.06%; LCMS Calculated for C₂₅H₂₄N₈O₂ (free base): 468.20; Observed: 469.40 (M+1).

### Examples 69-70

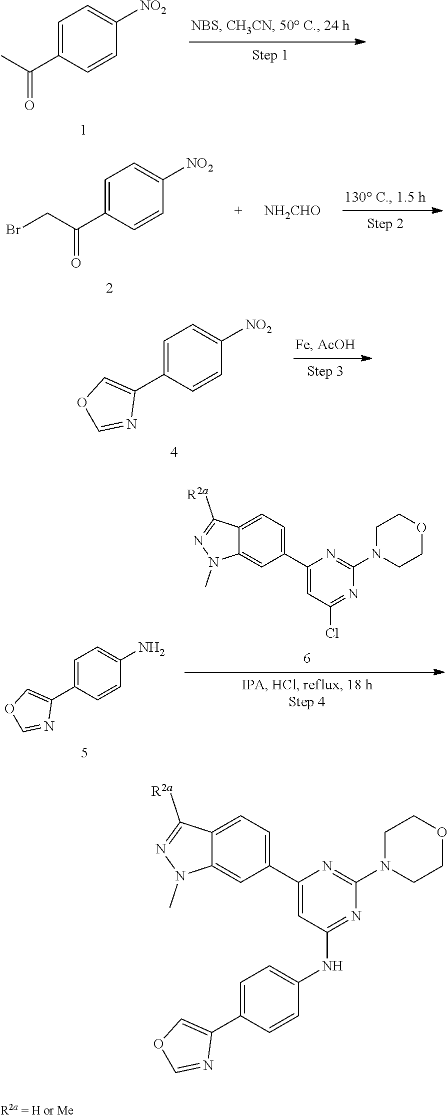

### Step 1: Synthesis of 2-bromo-1-(4-nitrophenyl)ethanone (2)

To a stirred solution of compound 1 (2 g, 1 eq) in acetonitrile (100 mL), NBS (3.23 g, 1 eq) was added followed by the addition of p-TSA (3.12 g, 1 eq). The reaction mixture was heated to 50° C. for 24 h. The progress of the reaction was monitored by TLC. After completion of the reaction, the reaction mixture was evaporated to dryness. The residue was basified with saturated sodium bicarbonate solution and extracted with dichloromethane (3×25 mL). Combined organic extracts were washed with brine, dried over anhydrous sodium sulfate and evaporated under reduced pressure to afford the title compound 2. ¹H NMR (400 MHz, CDCl₃) δ: 8.35 (d, J=8.4 Hz, 2H), 8.16 (d, J=9.2 Hz, 2H), 4.45 (s, 2H).

### Step 2: Synthesis of 4-(4-nitrophenyl)oxazole (4)

A stirred solution of compound 2 (1 g, 1 eq) in formamide 3 (2.55 g, 14.74 eq) was added at 130° C. for 1.5 h. The progress of the reaction was monitored by TLC. After completion of the reaction, the reaction mixture was diluted with water and extracted with ethyl acetate (2×50 mL). Combined organic extracts were washed with brine, dried over anhydrous sodium sulfate and concentrated under reduced pressure. The crude product was purified by column chromatography on silica gel 100-200 mesh using 20% EtOAc-hexane to afford the title compound 4. ¹H NMR (400 MHz, CDCl₃) δ: 8.29 (d, J=8.8 Hz, 2H), 8.10 (s, 1H), 7.99 (s, 1H), 7.92 (d, J=8.8 Hz, 2H).

### Step 3: Synthesis of 4-(oxazol-4-yl)aniline (5)

To a stirred solution of compound 4 (0.7 g, 1 eq) in acetic acid (10 mL), Iron powder (2.063 g) was added in one portion and the reaction mixture was stirred at room temperature for 3 h. The progress of the reaction was monitored by TLC. After completion of the reaction, the reaction mixture was filtered through celite and evaporated to dryness. The residue was basified with saturated sodium bicarbonate solution and extracted with ethyl acetate (2×25 mL). Combined organic extracts were washed with brine, dried over anhydrous sodium sulfate and evaporated under reduced pressure. The crude product was purified by column chromatography on silica gel 100-200 mesh using 30% EtOAc-hexane to afford the title compound 5. LCMS (m/z): 161.00 (M+1).

### Step 4

6-(1-methyl-1H-indazol-6-yl)-2-morpholino-N-(4-(oxazol-4-yl)phenyl)pyrimidin-4-amine: The title compound has been synthesized by following the general procedure described above for displacement reaction (IPA, Conc. HCl) by using corresponding chloro compound 6 and amino compound 5. ¹H NMR (400 MHz, DMSO-d₆) δ: 9.57 (s, 1H), 8.54 (d, J=1.0 Hz, 1H), 8.44 (d, J=0.9 Hz, 1H), 8.24 (d, J=1.2 Hz, 1H), 8.10 (d, J=1.0 Hz, 1H), 7.89-7.72 (m, 6H), 6.74 (s, 1H), 4.13 (s, 3H), 3.83-3.74 (m, 8H); HPLC purity: 99.04%; LCMS Calculated for C₂₅H₂₃N₇O₂: 453.19; Observed: 454.35 (M+1).

6-(1,3-dimethyl-1H-indazol-6-yl)-2-morpholino-N-(4-(oxazol-4-yl)phenyl) pyrimidin-4-amine:The title compound has been synthesized by following the general procedure described above for displacement reaction (IPA, Conc. HCl) by using corresponding chloro compound 6 and amine 5. ¹H NMR (400 MHz, DMSO-d₆) δ: 9.55 (s, 1H), 8.54 (d, J=1.0 Hz, 1H), 8.43 (d, J=1.0 Hz, 1H), 8.16 (s, 1H), 7.88-7.68 (m, 6H), 6.73 (s, 1H), 4.04 (s, 3H), 3.86-3.70 (m, 8H), 2.45 (s, 3H); HPLC purity: 93.56%; LCMS Calculated for C₂₆H₂₅N₇O₂: 467.21; Observed: 468.30 (M+1).

### Examples 71-72

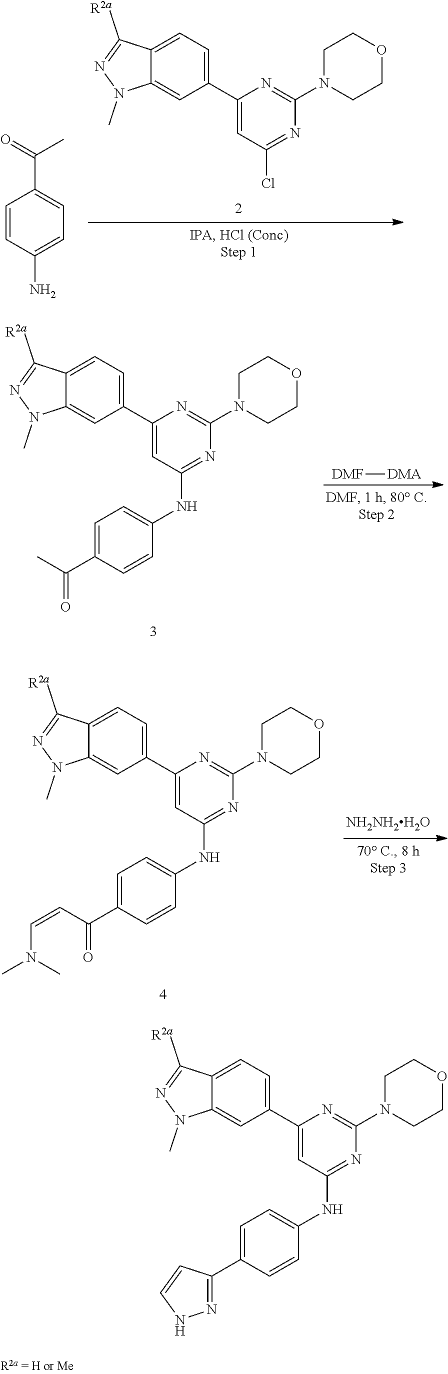

### Step 1

1-(4-((6-(1-methyl-1H-indazol-6-yl)-2-morpholinopyrimidin-4-yl)amino)phenyl) ethan-1-one (3): The title compound has been synthesized by following the general procedure described above for displacement reaction (IPA, Conc. HCl) by using corresponding chloro compound 2 and amine 1. LCMS (m/z): 429.35 (M+1).

1-(4-((6-(1,3-dimethyl-1H-indazol-6-yl)-2-morpholinopyrimidin-4-yl)amino)phenyl) ethan-1-one (3): The title compound has been synthesized by following the general procedure described above for displacement reaction (IPA, Conc. HCl) by using corresponding chloro compound 2 and amine 1. LCMS (m/z): 443.35 (M+1).

### Step 2

(Z)-3-(dimethylamino)-1-(4-46-(1-methyl-1H-indazol-6-yl)-2-morpholinopyrimidin-4-yl) amino) phenyl) prop-2-en-1-one (4): To a stirred solution of corresponding compound 3 (0.5 g, 1 eq) in DMF (7 mL), dimethylformamide dimethyl acetal (DMF-DMA) (0.2 mL, 1.2 eq) was added and heated at 80° C. for 1.5 h. The progress of the reaction was monitored by TLC. After completion of the reaction, the reaction mixture was diluted with cold water and filtered. The solid was washed with water, dried under vacuum to afford title compound 4. LCMS (m/z): 484.35 (M+1).

((Z)-1-(4-((6-(1, 3-dimethyl-1H-indazol-6-yl)-2-morpholinopyrimidin-4-yl) amino) phenyl)-3-(dimethylamino) prop-2-en-1-one (4): To a stirred solution of corresponding compound 3 (0.4 g, 1 eq) in DMF (5 mL), dimethylformamide dimethyl acetal (10 mL) was added and heated at 120° C. for 1.5 h. The progress of the reaction was monitored by TLC. After completion of the reaction, the reaction mixture was diluted with cold water and filtered. The solid was washed with water, dried under vacuum to afford title compound 4. LCMS (m/z): 498.40 (M+1).

### Step 3

N-(4-(1H-pyrazol-3-yl)phenyl)-6-(1-methyl-1H-indazol-6-yl)-2-morpholinopyrimidin-4-amine: To a stirred solution of corresponding compound 4 (0.3 g, 1 eq) in hydrazine hydrate (5 mL) was heated at 120° C. for 8 h. The progress of the reaction was monitored by TLC. After completion of the reaction, the reaction mixture was evaporated to dryness. The residue was diluted with water and extracted with ethyl acetate (2×25 mL). Combined organic extracts were washed with brine, dried over anhydrous sodium sulfate and evaporated under reduced pressure. The crude product was purified by preparative HPLC to afford title compound. ¹H NMR (400 MHz, DMSO-d₆) δ: 10.12 (s, 1H), 8.23 (s, 1H), 8.14 (s, 1H), 7.91 (d, J=8.5 Hz, 2H), 7.85-7.62 (m, 6H), 6.78-6.67 (m, 2H), 4.14 (s, 3H), 3.84-3.76 (m, 8H); HPLC purity: 97.73%; LCMS Calculated for C₂₅H₂₄N₈O (free base): 452.21; Observed: 453.35 (M+1).

N-(4-(1H-pyrazol-3-yl)phenyl)-6-(1,3-dimethyl-1H-indazol-6-yl)-2-morpholino pyrimidin-4-amine: The title compound has been synthesized by following same procedure described above. ¹H NMR (400 MHz, DMSO-d₆) δ: 10.45 (s, 1H), 8.15 (s, 1H), 7.91-7.80 (m, 3H), 7.80-7.73 (m, 3H), 7.57 (d, J=8.4 Hz, 1H), 6.80-6.70 (m, 2H), 4.06 (s, 3H), 3.90-3.76 (m, 8H), 2.52 (s, 3H); HPLC purity: 96.22%; LCMS Calculated for C₂₆H₂₆N₈O: 466.22; Observed: 467.35 (M+1).

### Examples 73-76

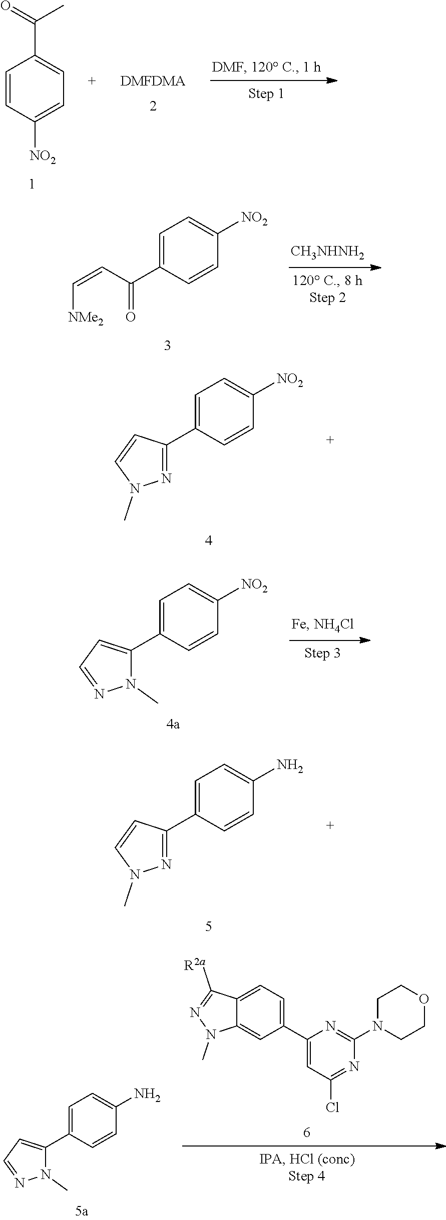

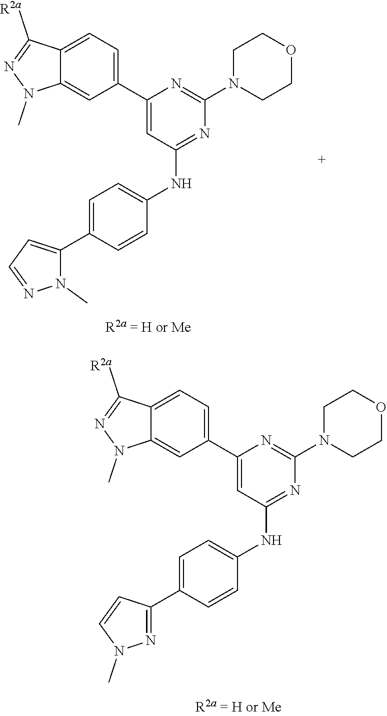

### Step 1: Synthesis of (Z)-3-(dimethylamino)-1-(4-nitrophenyl) prop-2-en-1-one (3)

To a stirred solution of compound 1 (5 g, 1 eq) in DMF (20 mL), dimethylformamide dimethylacetal 2 (40 mL) was added and heated at 120° C. for 1 h. The progress of the reaction was monitored by TLC. After completion of the reaction, the reaction mixture was diluted with cold water and filtered. The solid was washed with water, dried under vacuum to afford title compound 3. LCMS (m/z); 221.10 (M+1).

### Step 2: Synthesis of 1-methyl-3-(4-nitrophenyl)-1H-pyrazole (4) and 1-methyl-5-(4-nitrophenyl)-1H-pyrazole (4a)

A stirred solution of compound 3 (2 g, 1 eq) in methyl hydrazine (10 mL) was heated at 100° C. for 18 h. The progress of the reaction was monitored by TLC. After completion of the reaction, the reaction mixture was evaporated to dryness. The residue was diluted with water and extracted with ethyl acetate (3×25 mL). Combined organic extracts were washed with brine, dried over anhydrous sodium sulfate and evaporated under reduced pressure to afford mixture of compounds 4 & 4a.

### Step 3: Synthesis of 4-(1-methyl-1H-pyrazol-3-yl) aniline (5) and 1-methyl-5-(4-nitrophenyl)-1H-pyrazole (5a)

To a stirred solution of compound 4 & 4a (1 g, 1 eq) in ethanol (30 mL), iron powder (1.37 g, 5 eq), water (10 mL) and ammonium chloride (1.31 g, 5 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (50 mL), washed with brine, dried over anhydrous sodium sulfate and evaporated under reduced pressure to afford the mixture of compounds 5 & 5a. LCMS (m/z): 174.05 (M+1).

### Step 4

6-(1-methyl-1H-indazol-6-yl)-N-(4-(1-methyl-1H-pyrazol-5-yl)phenyl)-2-morpholinopyrimidin-4-amine: The title compound has been synthesized by following the general procedure described above for displacement reaction (IPA, Conc. HCl) by using corresponding chloro compound 6 and amino compound 5 and 5a. The individual regioisomers were separated by Preparative HPLC. ¹H NMR (400 MHz, DMSO-d₆) δ: 9.89 (s, 1H), 8.24 (d, J=1.3 Hz, 1H), 8.12 (s, 1H), 7.92-7.78 (m, 3H), 7.74 (d, J=8.5 Hz, 1H), 7.56-7.48 (m, 2H), 7.46 (d, J=1.9 Hz, 1H), 6.76 (s, 1H), 6.38 (d, J=1.9 Hz, 1H), 4.14 (s, 3H), 3.87 (s, 3H), 3.84-3.75 (m, 8H); HPLC purity: 97.25%; LCMS Calculated for C₂₆H₂₆N₈O (free base): 466.22; Observed: 467.40 (M+1).

6-(1-methyl-1H-indazol-6-yl)-N-(4-(1-methyl-1H-pyrazol-3-yl)phenyl)-2-morpholino pyrimidin-4-amine: ¹H NMR (400 MHz, DMSO-d₆) δ: 9.50 (s, 1H), 8.24 (s, 1H), 8.10 (s, 1H), 7.85 (d, J=8.4 Hz, 1H), 7.82-7.67 (m, 6H), 6.72 (s, 1H), 6.63 (d, J=2.2 Hz, 1H), 4.13 (s, 3H), 3.86 (s, 3H), 3.82-3.74 (m, 8H); HPLC purity: 98.07%; LCMS Calculated for C₂₆H₂₆N₈O: 466.22; Observed: 467.35 (M+1).

6-(1,3-dimethyl-1H-indazol-6-yl)-N-(4-(1-methyl-1H-pyrazol-5-yl)phenyl)-2-morpholino pyrimidin-4-amine: The title compound has been synthesized by following the general procedure described above for displacement reaction (IPA, Conc. HCl) by using corresponding chloro compound 6 and amino compound 5 and 5a. The individual regioisomers were separated by Preparative HPLC. ¹H NMR (400 MHz, DMSO-d₆) δ: 10.20 (s, 1H), 8.16 (s, 1H), 7.86 (dd, J=8.5, 3.1 Hz, 3H), 7.58 (dd, J=22.3, 8.2 Hz, 3H), 7.47 (d, J=1.9 Hz, 1H), 6.81 (s, 1H), 6.40 (d, J=1.9 Hz, 1H), 4.05 (s, 3H), 3.88 (s, 3H), 3.85-3.75 (m, 8H), 2.52 (s, 3H); HPLC purity: 99.57%; LCMS Calculated for C₂₇H₂₈N₈O (free base): 480.24; Observed: 481.30 (M+1).

6-(1,3-dimethyl-1H-indazol-6-yl)-N-(4-(1-methyl-1H-pyrazol-3-yl)phenyl)-2-morpholino pyrimidin-4-amine: ¹H NMR (400 MHz, DMSO-d₆) δ: 10.36 (s, 1H), 8.14 (s, 1H), 7.90-7.69 (m, 6H), 7.58 (d, J=8.5 Hz, 1H), 6.75 (s, 1H), 6.66 (d, J=2.3 Hz, 1H), 4.05 (s, 3H), 3.87 (s, 3H), 3.85-3.76 (m, 8H), 2.52 (s, 3H); HPLC purity: 98.12%; LCMS Calculated for C₂₇H₂₈N₈O (freebase): 480.24; Observed: 481.35 (M+1).

### Examples 77-78

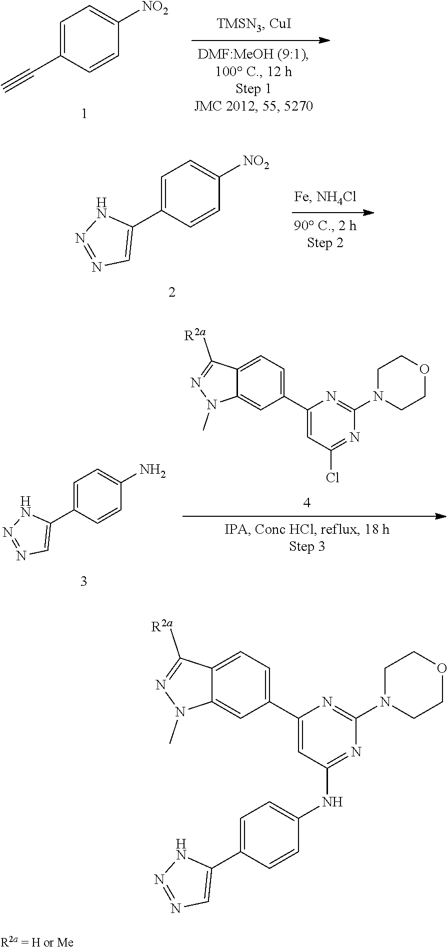

### Step 1: Synthesis of 5-(4-nitrophenyl)-1H-1,2,3-triazole (2)

To a stirred solution of compound 1 (0.7 g, 1 eq) and CuI (0.045 mg, 0.05 g) in DMF: methanol (9:1; 10 mL), TMSN₃ (0.821 g, 1.5 eq) was added. The reaction mixture was heated at 100° C. for 18 h. The progress of the reaction was monitored by TLC. After completion of the reaction, the reaction mixture was diluted with water and extracted with ethyl acetate (2×25 mL). Combined organic extracts were washed with brine, dried over anhydrous sodium sulfate and evaporated under reduced pressure to afford the title compound 2. LCMS (m/z): 191.10 (M+1).

### Step 2: Synthesis of 4-(1H-1,2,3-triazol-5-yl)aniline (3)

To a stirred solution of compound 2 (0.8 g, 1 eq) in ethanol (30 mL), iron powder (1.11 g, 5 eq), water (15 mL) and ammonium chloride (1.17 g. 5 eq) were added slowly. The reaction mixture was heated at 90° C. for 2 h. The progress of the reaction was monitored by TLC. After completion of the reaction, the reaction mixture was filtered through celite and evaporated under reduced pressure. The residue was dissolved in ethyl acetate (50 mL), washed with brine, dried over anhydrous sodium sulfate and evaporated under reduced pressure to afford the title compound 3. LCMS (m/z): 161.00 (M+1).

### Step 3

N-(4-(1H-1,2,3-triazol-5-yl)phenyl)-6-(1-methyl-1H-indazol-6-yl)-2-morpholino pyrimidin-4-amine: The title compound has been synthesized by following the general procedure described above for displacement reaction (IPA, Conc. HCl) by using corresponding chloro compound 4 and amine 3. ¹H NMR (400 MHz, DMSO-d₆) δ: 8.29 (s, 1H), 8.24 (s, 1H), 8.15 (s, 1H), 7.90 (dd, J=15.1, 8.4 Hz, 3H), 7.81 (d, J=8.4 Hz, 2H), 7.67 (d, J=8.5 Hz, 1H), 6.77 (s, 1H), 4.15 (s, 3H), 3.82-3.69 (m, 8H); HPLC purity: 99.98%; LCMS Calculated for C₂₄H₂₃N₉O (free base): 453.20; Observed: 454.25 (M+1).

N-(4-(1H-1,2,3-triazol-5-yl)phenyl)-6-(1,3-dimethyl-1H-indazol-6-yl)-2-morpholino pyrimidin-4-amine: The title compound has been synthesized by following the general procedure described above for displacement reaction (IPA, Conc. HCl) by using corresponding chloro compound 4 and amine 3. ¹H NMR (400 MHz, DMSO-d₆) δ: 15.05 (s, 1H), 9.57 (s, 1H), 8.25 (s, 1H), 8.17 (s, 1H), 7.87-7.69 (m, 6H), 6.74 (s, 1H), 4.04 (s, 3H), 3.83-3.74 (m, 8H), 2.50 (s, 3H); HPLC purity: 96.37%; LCMS Calculated for C₂₅H₂₅N₉O: 467.22; Observed: 468.25 (M+1).

### Examples 79

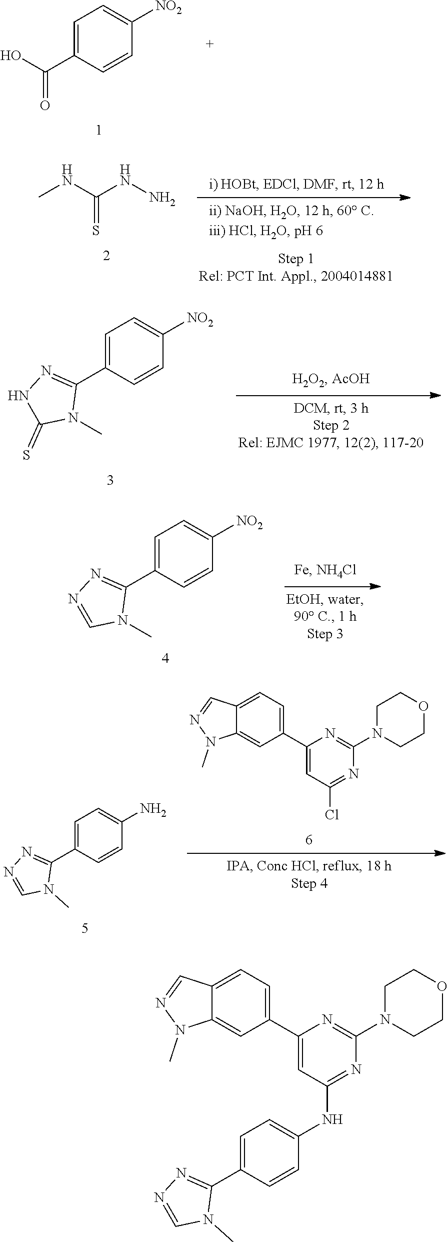

### Step 1: Synthesis of 4-methyl-3-(4-nitrophenyl)-1H-1,2,4-triazole-5(4H)-thione

To a stirred solution of compound 1 (0.5 g, 1 eq) and compound 2 (0.314 g, 1 g) in DMF (10 mL), EDCI (0.571 g, 1 eq) was added followed by the addition of HOBt (0.403 g, 1 eq). The reaction mixture was stirred at room temperature for 18 h. The progress of the reaction was monitored by TLC. After completion of the reaction, the reaction mixture was diluted with water and extracted with ethyl acetate (2×25 mL). Combined organic extracts were washed with brine, dried over anhydrous sodium sulfate and evaporated under reduced pressure. The residue was dissolved in 5% NaOH solution and heated at 60° C. for 18 h. the reaction mixture was cooled to 0° C. and acidified to pH 6 using ¹N HCl. The aqueous layer was saturated with sodium chloride and extracted with ethyl acetate (2×25 mL). Combined organic extracts were evaporated under reduced pressure to afford the title compound 3. ¹H NMR (400 MHz, DMSO-d₆) δ: 14.12 (s, 1H), 8.38 (d, J=8.4 Hz, 2H), 8.38 (d, J=8.0 Hz, 2H) 3.58 (s, 3H).

### Step 2: Synthesis of 4-methyl-3-(4-nitrophenyl)-4H-1,2,4-triazole (4)

To a stirred solution of compound 3 (0.1 g, 1 eq) in dichloromethane (10 mL), H₂O₂ (0.032 g, 2.2 eq) was added slowly at 0° C. followed by the addition of acetic acid at same temperature. The reaction mixture was stirred at room temperature for 3 h. The progress of the reaction was monitored by TLC. After completion of the reaction, the reaction mixture was basified to pH 10 with aq. sodium hydroxide and extracted with dichloromethane (2×10 mL). Combined organic extracts were washed with brine, dried over anhydrous sodium sulfate and evaporated under reduced pressure to afford the title compound 4. LCMS (m/z): 205.00 (M+1).

### Step 3: Synthesis of 4-(4-methyl-4H-1,2,4-triazol-3-yl)aniline (5)

To a stirred solution of compound 4 (0.05 g, 1 eq) in ethanol (5 mL), iron powder (0.068 g, 5 eq), water (2 mL) and ammonium chloride (0.065 g. 5 eq) were added slowly. The reaction mixture was heated at 90° C. for 1 h. The progress of the reaction was monitored by TLC. After completion of the reaction, the reaction mixture was filtered through celite and evaporated under reduced pressure. The residue was diluted with water and extracted with 10% methanol in dichloromethane and evaporated under reduced pressure to afford the title compound 5. LCMS (m/z): 174.95 (M+1).

### Step 4

6-(1-methyl-1H-indazol-6-yl)-N-(4-(4-methyl-4H-1,2,4-triazol-3-yl)phenyl)-2-morpholinopyrimidin-4-amine: The title compound has been synthesized by following the general procedure described above for displacement reaction (IPA, Conc. HCl) by using corresponding chloro compound 6 and amine 5. ¹H NMR (400 MHz, DMSO-d₆) δ: 10.32 (s, 1H), 9.32 (s, 1H), 8.26 (d, J=1.2 Hz, 1H), 8.13 (d, J=1.0 Hz, 1H), 8.08-7.99 (m, 2H), 7.92-7.82 (m, 3H), 7.76 (dd, J=8.5, 1.4 Hz, 1H), 6.89 (s, 1H), 3.92 (s, 3H), 3.86 (s, 3H), 3.84-3.76 (m, 8H); HPLC purity: 95.56%; LCMS Calculated for C₂₅H₂₅N₉O (free base): 467.22; Observed: 468.35 (M+1).

### Example 80

### Synthesis of 6-(1,3-dimethyl-1H-indazol-6-yl)-N-(4-(5-methyl-4H-1,2,4-triazol-3-yl)phenyl)-2-morpholinopyrimidin-4-amine

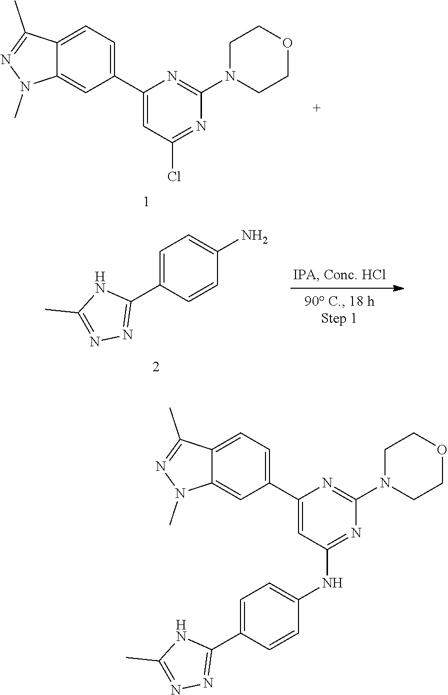 [SMILES: Cc1nn(C)c2cc(-c3cc(Cl)nc(N4CCOCC4)n3)ccc12 ; Cc1nnc(-c2ccc(N)cc2)[nH]1 ; Cc1nnc(-c2ccc(Nc3cc(-c4ccc5c(C)nn(C)c5c4)nc(N4CCOCC4)n3)cc2)[nH]1]

### Step 1: Synthesis of 6-(1,3-dimethyl-1H-indazol-6-yl)-N-(4-(5-methyl-4H-1,2,4-triazol-3-yl)phenyl)-2-morpholinopyrimidin-4-amine

The title compound has been synthesized by following the general procedure described above for displacement reaction (IPA, Conc. HCl) by using chloro compound 1 and amine 2. ¹H NMR (400 MHz, CDCl₃) δ: 10.60 (s, 1H),8.07-7.97 (m, 3H), 7.65 (s, 2H), 7.55 (d, J=8.2 Hz, 2H), 6.73 (s, 1H), 6.57 (s, 1H), 4.06 (s, 3H), 3.92-3.82 (m, 8H), 2.56 (s, 3H), 2.57 (s, 3H); HPLC purity: 99.08%; LCMS Calculated for C₂₆H₂₇N₉O: 481.23; Observed: 482.40 (M+1).

### Example 81

### Synthesis of N-(4-(1H-imidazol-4-yl)phenyl)-6-(1,3-dimethyl-1H-indazol-6-yl)-2-morpholinopyrimidin-4-amine

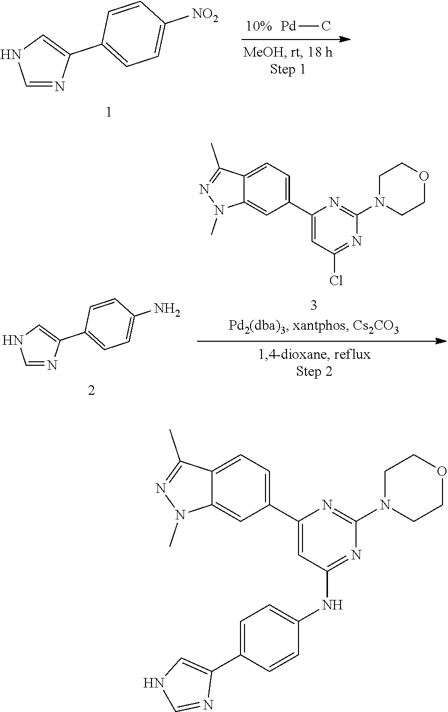

### Step 1: Synthesis of 4-(1H-imidazol-4-yl)aniline (2)

To a stirred solution of compound 1 (0.7 g, 1 eq) in methanol (10 mL), 10% Pd—C (0.1 g) was added under nitrogen atmosphere. The reaction mixture was stirred at room temperature for 18 h under hydrogen balloon pressure. The progress of the reaction was monitored by TLC. After completion of the reaction, the reaction mixture was filtered through celite and evaporated under reduced pressure. The crude product was purified by column chromatography on silica gel 100-200 mesh using 5% MeOH-DCM to afford the title compound 2. LCMS (m/z): 160.00 (M+1).

### Step 2: Synthesis of N-(4-(1H-imidazol-4-yl)phenyl)-6-(1,3-dimethyl-1H-indazol-6-yl)-2-morpholino pyrimidin-4-amine

To a stirred solution of compound 3 (0.2 g, 1 eq) and compound 2 (0.093 g, 1 eq) in 1,4-dioxane (10 mL), cesium carbonate (0.285g, 1.5 eq) was added and purged with argon for 10 min, followed by the addition of xantphos (0.033 g,0.15 eq) and purged with argon for additional 5 min. Pd₂(dba)₃ (0.053 g,0.1 eq) was added and stirred at 100° C. for 18 h. The progress of the reaction was monitored by TLC. After completion of the reaction, the reaction mixture was filtered through celite and evaporated to dryness. The residue was dissolved in ethyl acetate (50 mL), washed with water, brine, dried over anhydrous sodium sulfate and evaporated under reduced pressure. The crude product was purified by preparative HPLC to afford the desired product. ¹H NMR (400 MHz, MeOD) δ: 8.97 (d, J=1.4 Hz, 1H), 8.10 (s, 1H), 7.85 (q, J=8.5 Hz, 4H), 7.78-7.71 (m, 2H), 7.67 (dd, J=8.5, 1.4 Hz, 1H), 6.66 (s, 1H), 4.08 (s, 3H), 3.96-3.84 (m, 8H),2.58 (s, 3H); HPLC purity: 98.24%; LCMS Calculated for C₂₆H₂₆N₈O: 466.22; Observed: 467.25 (M+1).

### Example 82

### N-(6-(1,3-dimethyl-1H-indazol-6-yl)-2-morpholinopyrimidin-4-yl)-1H-indazol-5-amine

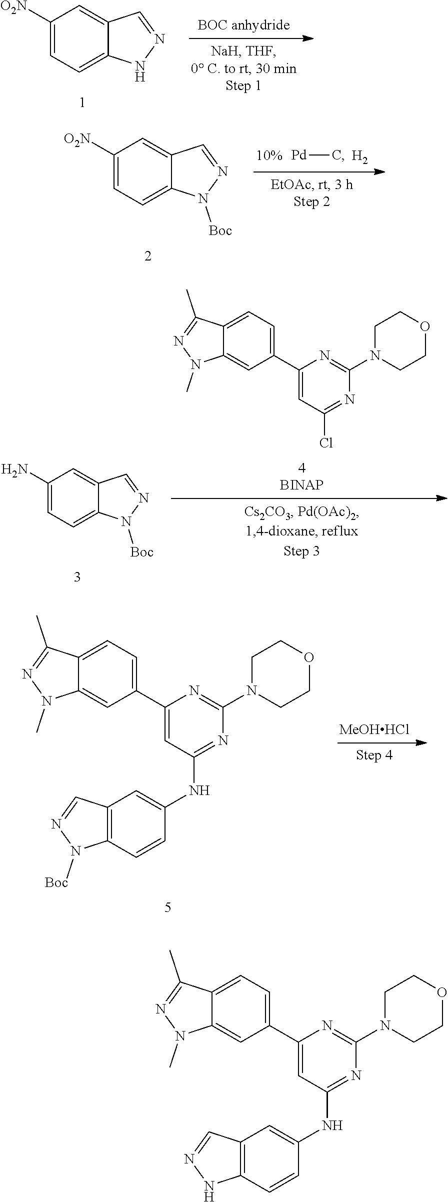

### Step 1: Synthesis of tert-butyl 5-nitro-1H-indazole-1-carboxylate (2)

To a stirred solution of compound 1 (1 g, 1 eq) in THF (10 mL), NaH (0.161 g, 1.1 eq) was added under nitrogen atmosphere at 0° C. and stirred for 30 min. Boc-anhydride (1.6 g, 1.2 eq) was added at same temperature and stirred at room temperature for 30 min. The progress of the reaction was monitored by TLC. After completion of the reaction, the reaction mixture was diluted with ice water extracted with ethyl acetate (3×25 mL). Combined organic extracts were washed with brine, dried over anhydrous sodium sulfate and evaporated under reduced pressure to afford the title compound 2.

### Step 2: Synthesis of tert-butyl 5-amino-1H-indazole-l-carboxylate (3)

To a stirred solution of compound 2 (1 g, 1 eq) in ethyl acetate (30 mL), 10% Pd—C (0.28 g) was added under nitrogen atmosphere. The reaction mixture was stirred at room temperature for 3 h under hydrogen atmosphere (balloon pressure). The progress of the reaction was monitored by TLC. After completion of the reaction, the reaction mixture was filtered through celite and evaporated under reduced pressure to afford the title compound 3.

### Step 3: Synthesis of tert-butyl 5-((6-(1,3-dimethyl-1H-indazol-6-yl)-2-morpholinopyrimidin-4-yl) amino)-1H-indazole-1-carboxylate (5)

The title compound has been synthesized by following the General procedure for Buchwald Coupling described above using chloro compound 4 and amino compound 3. LCMS (m/z): 541.45 (M+1).

### Step 4: Synthesis of N-(6-(1,3-dimethyl-1H-indazol-6-yl)-2-morpholinopyrimidin-4-yl)-1H-indazol-5-amine

To a stirred solution of compound 5 (0.09 g, 1 eq) in methanol (5 mL), methanolic HCl (3 mL) was added and stirred at room temperature for 1 h. The progress of the reaction was monitored by TLC. After completion of the reaction, the reaction mixture was evaporated to dryness. The crude product was purified by washing with diethyl ether to afford title compound as a HCl salt. ¹H NMR (400 MHz, MeOD) δ: 8.26 (s, 1H), 8.15 (s, 1H), 8.03 (s, 1H), 7.93 (d, J=8.6 Hz, 1H), 7.72-7.64 (m, 2H), 7.50 (s, 1H), 6.66-6.61 (s, 1H), 4.11 (s, 3H), 3.92-3.80 (m, 8H), 2.60 (s, 3H); HPLC purity: 95.11%; LCMS Calculated for C₂₄H₂₄N₈₀ (free base): 440.21; Observed: 441.35 (M+1).

### Example 83

### Synthesis of N-(4-(1,2,4-oxadiazol-5-yl)phenyl)-6-(1,3-dimethyl-1H-indazol-6-yl)-2-morpholino pyrimidin-4-amine

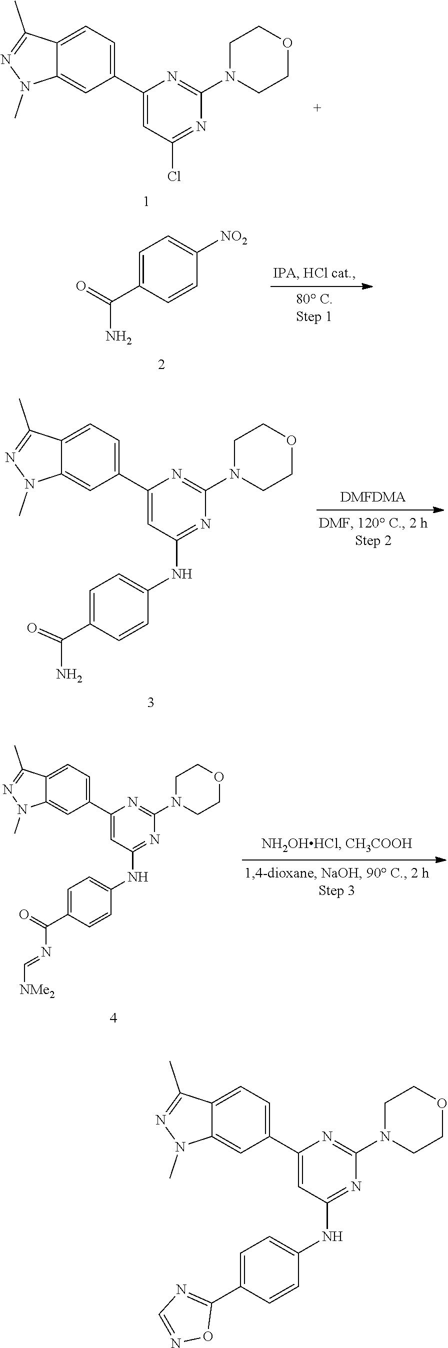

### Step 1: Synthesis of 4-((6-(1, 3-dimethyl-1H-indazol-6-yl)-2-morpholinopyrimidin-4-yl) amino) benzamide (3)

The title compound has been synthesized by following the general procedure described above for displacement reaction (IPA, Conc. HCl) by using chloro compound 1 and amine 2. LCMS (m/z): 444.30 (M+1).

### Step 2: Synthesis of (E)-4-((6-(1,3-dimethyl-1H-indazol-6-yl)-2-morpholinopyrimidin-4-yl)amino)-N-((dimethylamino)methylene)benzamide (4)

To a stirred solution of compound 3 (0.6 g, 1 eq) in DMF (5 mL), DMFDMA (10 mL) was added and heated at 120° C. for 2 h. The progress of the reaction was monitored by TLC. After completion of the reaction, the reaction mixture was diluted with cold water and filtered. The solid was washed with water, dried under vacuum to afford title compound 4. LCMS (m/z): 499.35 (M+1).

### Step 3: Synthesis of N-(4-(1,2,4-oxadiazol-5-yl)phenyl)-6-(1,3-dimethyl-1H-indazol-6-yl)-2-morpholino pyrimidin-4-amine

To a stirred solution of compound 4 (0.4 g, 1 eq) in 1,4-dioxane (10 mL), hydroxylamine hydrochloride (0.067 g,1.2 eq), 5N NaOH (0.19 mL) and acetic acid (5 mL) were added and heated at 90° C. for 2 h. The progress of the reaction was monitored by TLC. After completion of the reaction, the reaction mixture was diluted with cold water. The precipitated solid was filtered; residue was washed with water and dried under vacuum. The crude product was purified by preparative HPLC to afford the title compound. ¹H NMR (400 MHz, DMSO-d₆) δ: 9.96 (s, 1H), 9.02 (d, J=1.0 Hz, 1H), 8.18 (s, 1H), 8.10 (d, J=8.5 Hz, 2H), 7.97 (d, J=8.5 Hz, 2H), 7.83-7.70 (m, 2H), 6.79 (s, 1H), 4.04 (s, 3H), 3.84-3.71 (m, 8H), 2.46 (s, 3H); HPLC purity: 98.24%; LCMS Calculated for C₂₅H₂₄N₈O₂: 468.20; Observed: 469.25 (M+1).

### Example 84

### Synthesis of 6-(1,3-dimethyl-1H-indazol-6-yl)-2-morpholino-N-(4-(oxazol-2-yl)phenyl)pyrimidin-4-amine

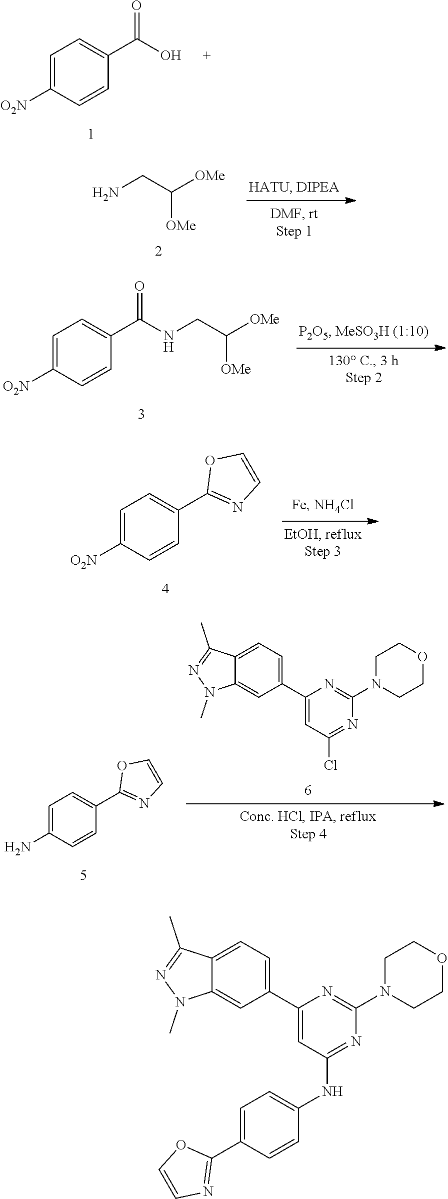

### Step 1: Synthesis of N-(2,2-dimethoxyethyl)-4-nitrobenzamide (3)

To a stirred solution of compound 1 (1.5 g, 1 eq) in DMF (15 mL), compound 2 (1.22 mL, 1.2 eq), HATU (5.1 g, 1.2 eq) and DIPEA (3 mL, 2 eq) were added and stirred at room temperature for 1 h. The progress of the reaction was monitored by TLC. After completion of the reaction, the reaction mixture was quenched with water and extracted with ethyl acetate (3×50 mL). Combined organic extracts were washed with brine, dried over anhydrous sodium sulfate and evaporated under reduced pressure to afford the title compound 3. ¹H NMR (400 MHz, DMSO-d₆) δ: 8.90 (t, J=5.2 Hz, 1H), 8.32 (d, J=8.8 Hz, 2H), 8.07 (d, J=8.8 Hz, 2H), 4.53 (t, J=5.2 Hz, 1H), 3.99 (t, J=5.6 Hz, 2H), 3.31 (s, 6H).

### Step 2: Synthesis of 2-(4-nitrophenyl) oxazole (4)

A stirred mixture of compound 3 (0.5 g, 1 eq) and P205: methane sulphonic acid (1:10; 11 mL) was heated at 130° C. for 5 h. The progress of the reaction was monitored by TLC. After completion of the reaction, the reaction mixture was quenched with aqueous sodium bicarbonate solution and extracted with ethyl acetate (2×25 mL). Combined organic extracts were washed with brine, dried over anhydrous sodium sulfate and evaporated under reduced pressure to afford title compound 4. LCMS (m/z): 190.95 (M+1).

### Step 3: Synthesis of 4-(oxazol-2-yl)aniline (5)

To a stirred solution of compound 4 (0.35 g, 1 eq) in ethanol (10 mL), iron powder (0.308 g, 4 eq), water (10 mL) and ammonium chloride (0.292 g, 4 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (50 mL), washed with brine, dried over anhydrous sodium sulfate and evaporated under reduced pressure to afford the title compound 5. LCMS (m/z): 160.90 (M+1).

### Step 4: Synthesis of 6-(1,3-dimethyl-1H-indazol-6-yl)-2-morpholino-N-(4-(oxazol-2-yl)phenyl)pyrimidin-4-amine

The title compound has been synthesized by following the general procedure described above for displacement reaction (IPA, Conc. HCl) by using chloro compound 6 and amine compound 5. ¹H NMR (400 MHz, MeOD) δ: 8.15-8.07 (m, 2H), 8.05-7.98 (m, 2H), 7.94 (d, J=8.4 Hz, 1H), 7.84 (d, J=8.5 Hz, 2H), 7.50 (dd, J=8.4, 1.4 Hz, 1H), 7.33 (d, J=0.9 Hz, 1H), 6.64 (s, 1H), 4.11 (s, 3H), 3.97-3.84 (m, 8H), 2.60 (s, 3H); HPLC purity: 97.85%; LCMS Calculated for C₂₆H₂₅N₇O₂ (free base): 467.21; Observed: 468.40 (M+1).

### Example 85

### Synthesis of N-(4-(4,5-dihydrooxazol-2-yl)phenyl)-6-(1,3-dimethyl-1H-indazol-6-yl)-2-morpholino pyrimidin-4-amine

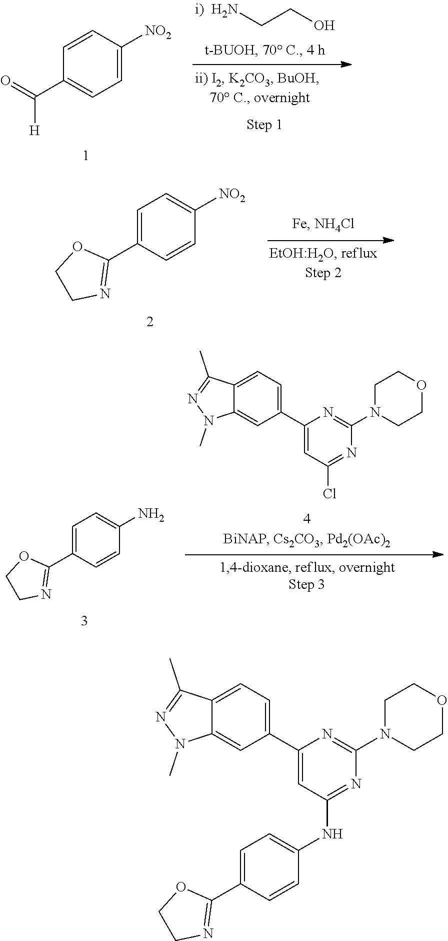

### Step 1: Synthesis of 2-(4-nitrophenyl)-4,5-dihydrooxazole (2)

To a stirred solution of compound 1 (1 g, 1 eq) in t-butanol (20 mL), 2-aminoethanol (0.44 g, 1.1 eq) was added and stirred at 70° C. for 4 h. Potassium carbonate (2.74 g, 3 eq) and Iodine (2.01 g, 1.2 eq) were added to the reaction mixture and again heated at 70° C. for overnight. The progress of the reaction was monitored by TLC. After completion of the reaction, the reaction mixture was evaporated to dryness. The residue was quenched with saturated sodium thiosulphate solution and extracted with ethyl acetate (3×25 mL). Combined organic extracts were washed with brine, dried over anhydrous sodium sulfate and evaporated under reduced pressure. The crude product was purified by column chromatography on silica gel 100-200 mesh using 30% EtOAc-hexane to afford the title compound 2. LCMS (m/z): 193.00 (M+1).

### Step 2: Synthesis of 4-(4,5-dihydrooxazol-2-yl)aniline (3)

To a stirred solution of compound 2 (0.3 g, 1 eq) in ethanol (5 mL), iron powder (0.305g, 3.5 eq), water (5 mL) and ammonium chloride (0.3 g 3.5 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (25 mL), washed with brine, dried over anhydrous sodium sulfate and evaporated under reduced pressure to afford the title compound 3. LCMS (m/z): 163.00 (M+1).

### Step 3: Synthesis of N-(4-(4,5-dihydrooxazol-2-yl)phenyl)-6-(1,3-dimethyl-1H-indazol-6-yl)-2-morpholinopyrimidin-4-amine

The title compound has been synthesized by using general procedure for Buchwald coupling using the chloro compound 4 and amine compound 3. ¹H NMR (400 MHz, CDCl₃) δ: 8.01 (s, 1H), 7.95 (d, J=8.3 Hz, 2H), 7.67 (s, 2H), 7.53 (d, J=8.5 Hz, 2H), 6.70 (s, 1H), 6.59 (s, 1H), 4.44 (t, J=9.4 Hz, 2H), 4.08 (t, J=9.5 Hz, 2H), 4.07 (s, 3H), 3.93-3.83 (m, 8H), 2.59 (s, 3H); HPLC purity: 91.06%; LCMS Calculated for C₂₆H₂₇N₇O₂: 469.22; Observed: 470.45 (M+1).

### Example 86

### Synthesis of N-(4-(4H-1,2,4-triazol-3-yl)phenyl)-6-(1,3-dimethyl-1H-indazol-6-yl)-2-morpholino pyrimidin-4-amine

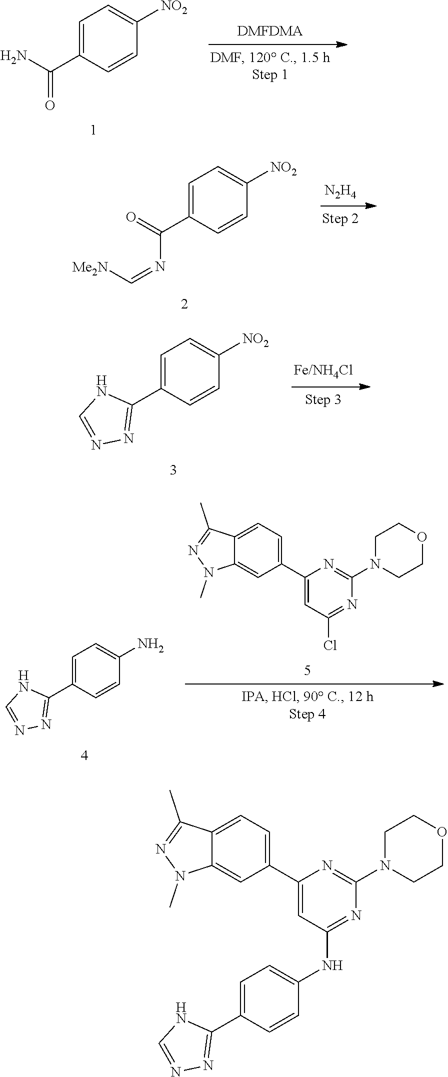

### Step 1: Synthesis of (Z)-N-((dimethylamino) methylene)-4-nitrobenzamide (2)

To a stirred solution of compound 1 (1 g, 1 eq) in DMF (1.5 mL), DMF DMA (3 mL) was added and heated at 120° C. for 1.5 h. The progress of the reaction was monitored by TLC. After completion of the reaction, the reaction mixture was diluted with cold water and filtered. The solid was washed with water, dried under vacuum to afford the title compound 2. LCMS (m/z): 222.00 (M+1).

### Step 2: Synthesis of 3-(4-nitrophenyl)-4H-1,2,4-triazole (3)

To a stirred solution of compound 2 (1.2 g, 1 eq) in acetic acid (10 mL), hydrazine hydrate (0.3 mL, 1.1 eq) was added and heated at 90° C. for 1.5 h. The progress of the reaction was monitored by TLC. After completion of the reaction, the reaction mixture was evaporated to dryness. The residue was neutralized with saturated sodium bicarbonate solution and extracted with ethyl acetate (3×25 mL). Combined organic extracts were washed with brine, dried over anhydrous sodium sulfate and evaporated under reduced pressure to afford the title compound 3. LCMS (m/z): 191.00 (M+1).

### Step 3: Synthesis of 4-(4H-1,2,4-triazol-3-yl)aniline (4)

To a stirred solution of compound 3 (0.05 g, 1 eq) in ethanol (5 mL), iron powder (0.074 g, 5 eq), water (3 mL) and ammonium chloride (0.069 g 5 eq) were added slowly. The reaction mixture was heated to reflux for 4 h. The progress of the reaction was monitored by TLC. After completion of the reaction, the reaction mixture was filtered through celite and evaporated under reduced pressure. The residue was dissolved in ethyl acetate (25 mL), washed with brine, dried over anhydrous sodium sulfate and evaporated under reduced pressure to afford the title compound 4. LCMS (m/z): 161.00 (M+1).

### Step 4: Synthesis of N-(4-(4H-1,2,4-triazol-3-yl)phenyl)-6-(1,3-dimethyl-1H-indazol-6-yl)-2-morpholino pyrimidin-4-amine

The title compound has been synthesized by following the general procedure described above for displacement reaction (IPA, Conc. HCl) by using chloro compound 5 and amine 4. ¹H NMR (400 MHz, DMSO-d₆) δ: 14.14-14.03 (m, 1H), 9.64 (s, 1H), 8.42-8.25 (m, 1H), 8.17 (s, 1H), 7.97 (d, J=8.3 Hz, 2H), 7.86-7.69 (m, 4H), 6.75 (s, 1H), 4.04 (s, 3H), 3.83-3.74 (m, 8H), 2.50 (s, 3H); HPLC purity: 99.02%; LCMS Calculated for C₂₅H₂₅N₉O: 467.22; Observed: 468.35 (M+1).

### Examples 87

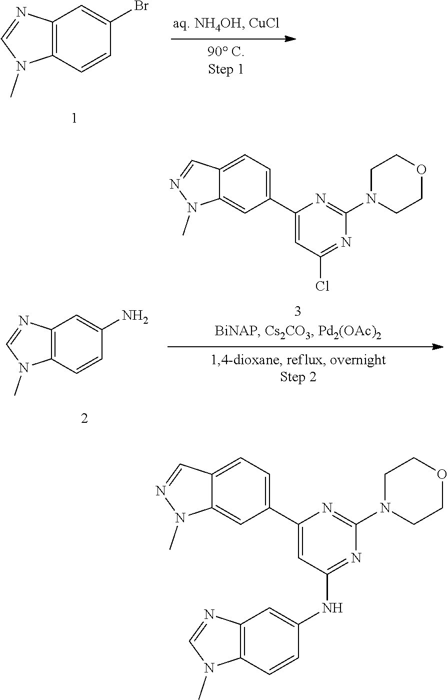

### Step 1: Synthesis of 1-methyl-1H-benzo[d]imidazol-5-amine (2)

To a stirred solution of compound 1 (1.6 g, 1 eq) in aq. ammonia solution (5 mL), copper chloride (catalytic amount) was added and heated at 90° C. in a sealed tube for 18 h. The progress of the reaction was monitored by TLC. After completion of the reaction, the reaction mixture was evaporated to dryness. The residue was stirred with 20% methanol in dichloromethane and filtered. The filtrate was evaporated under reduced pressure to afford the title compound 2. LCMS (m/z): 148.00 (M+1).

### Step 2

1-methyl-N-(6-(1-methyl-1H-indazol-6-yl)-2-morpholinopyrimidin-4-yl)-1H-benzo[d]imidazol-5-amine: The title compound has been synthesized by following the General procedure for Buchwald Coupling described above using corresponding chloro compound 3 and amine 2. ¹H NMR (400 MHz, DMSO-d₆) δ: 9.37 (s, 1H), 8.23 (d, J=1.2 Hz, 1H), 8.16-8.06 (m, 2H), 8.03 (s, 1H), 7.84 (d, J=8.5 Hz, 1H), 7.75 (dd, J=8.6, 1.3 Hz, 1H), 7.56-7.45 (m, 2H), 6.69 (s, 1H), 4.12 (s, 3H), 3.82 (s, 3H),3.81-3.72 (m, 8H); HPLC purity: 97.76%; LCMS Calculated for C₂₄H₂₄N₈O: 440.21; Observed: 441.30 (M+1).

### Examples 88-89

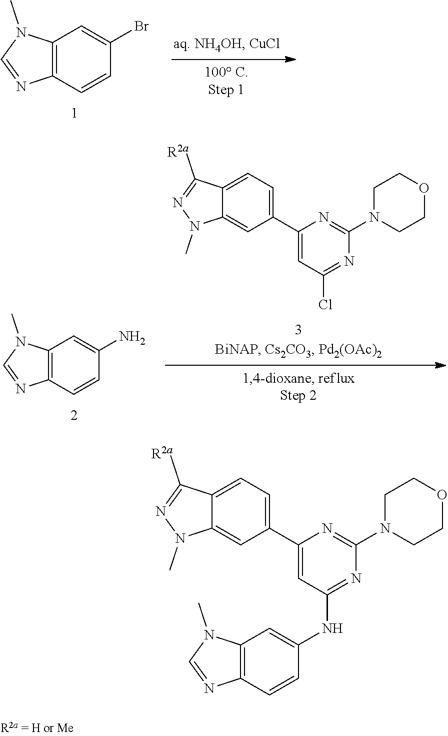

### Step 1: Synthesis of 1-methyl-1H-benzo[d]imidazol-6-amine (2)

To a stirred solution of compound 1 (0.2 g, 1 eq) in aq. ammonia solution (2 mL), copper chloride (catalytic amount) was added and heated at 100° C. for 5 h in a sealed tube. The progress of the reaction was monitored by TLC. After completion of the reaction, the reaction mixture was evaporated to dryness. The residue was stirred with 20% methanol in dichloromethane and filtered. The filtrate was concentrated under reduced pressure to afford title compound 2. LCMS (m/z): 148.00 (M+1).

### Step 2

1-methyl-N-(6-(1-methyl-1H-indazol-6-yl)-2-morpholinopyrimidin-4-yl)-1H-benzo[d]imidazol-6-amine: The title compound has been synthesized by following the General procedure for Buchwald Coupling described above using corresponding chloro compound 3 and amine 2. ¹H NMR (400 MHz, DMSO-d₆) δ: 9.54 (s, 1H), 8.32 (s, 1H), 8.24 (d, J=1.2 Hz, 1H), 8.09 (d, J=1.0 Hz, 2H), 7.85 (d, J=8.5 Hz, 1H), 7.77 (dd, J=8.5, 1.3 Hz, 1H), 7.57 (d, J=8.5 Hz, 1H), 7.20 (dd, J=8.6, 2.0 Hz, 1H), 6.74 (s, 1H), 4.13 (s, 3H), 3.87 (s, 3H), 3.82-3.71 (m, 8H); HPLC purity: 99.42%; LCMS Calculated for C₂₄H₂₄N₈O: 440.21; Observed: 441.35 (M +1).

N-(6-(1, 3-dimethyl-1H-indazol-6-yl)-2-morpholinopyrimidin-4-yl)-1-methyl-1H-benzo[d]imidazol-6-amine: The title compound has been synthesized by following the General procedure for Buchwald Coupling described above using corresponding chloro compound 3 and amine 2. ¹H NMR (400 MHz, DMSO-d₆) δ: 9.53 (s, 1H), 8.32 (s, 1H), 8.16 (s, 1H), 8.09 (s, 1H), 7.82-7.68 (m, 2H), 7.57 (d, J=8.6 Hz, 1H), 7.20 (dd, J=8.5, 2.0 Hz, 1H), 6.73 (s, 1H), 4.03 (s, 3H), 3.86 (s, 3H), 3.82-3.71 (m, 8H), 2.55 (s, 3H); HPLC purity: 98%; LCMS Calculated for C₂₅H₂₆N₈O: 454.22; Observed: 455.40 (M+1).

### Examples 90-95

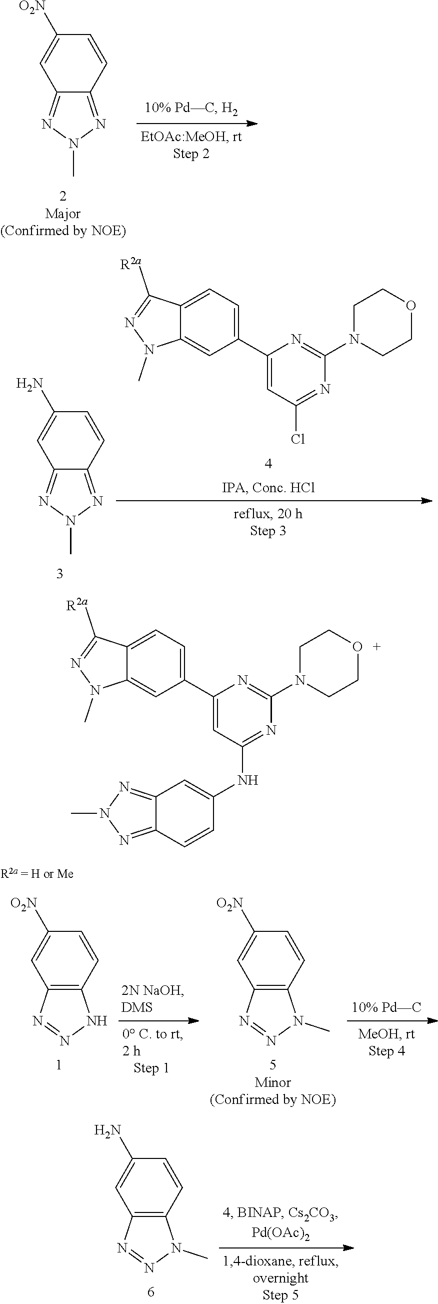

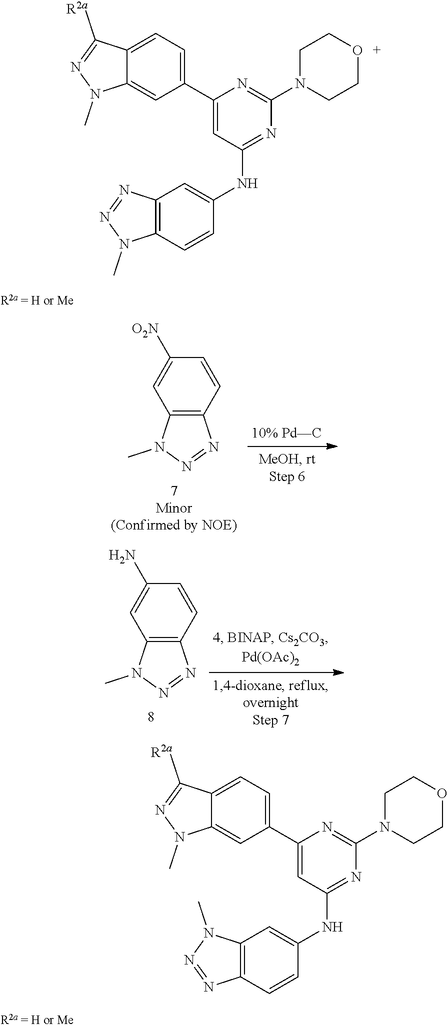

### Step 1: Synthesis of 2-methyl-5-nitro-2H-benzo[d][1,2,3]triazole (2), 1-methyl-5-nitro-1H-benzo[d] [1,2,3] triazole (5) and 1-methyl-6-nitro-1H-benzo[d][1,2,31triazole (7)

To a stirred solution of compound 1 (0.2 g, 1 eq) in 2 N NaOH solution (10 mL), water (5 mL) was added followed by the addition of DMS (3 mL, 2.53 eq). The reaction mixture was stirred at room temperature for 30 min. The reaction mixture was cooled at 0° C. and stirred for 2 h at room temperature. The progress of the reaction was monitored by TLC. After completion of the reaction, reaction mixture was filtered. The residue was dried under reduced pressure and purified by column chromatography on silica gel 100-200 mesh using 30% EtOAc-hexane to afford the title compounds 2, 5 and 7. LCMS (m/z): 178.90 (M+1).

### Step 2: Synthesis of 2-methyl-2H-benzo[d][1,2,3]triazol-5-amine (3)

To a stirred solution of compound 2 (0.4 g, 1 eq) in methanol: ethyl acetate (1:1; 40 mL), 10% Pd—C (0.1 g) was added and stirred at room temperature under hydrogen atmosphere (balloon pressure) for 18 h. The progress of the reaction was monitored by TLC. After completion of the reaction, the reaction mixture was filtered through celite and evaporated under reduced pressure to afford the title compound 3. LCMS (m/z): 149.00 (M+1).

### Step 3

2-methyl-N-(6-(1-methyl-1H-indazol-6-yl)-2-morpholinopyrimidin-4-yl)-2H-benzo[d][1,2,3]triazol-5-amine: The title compound has been synthesized by following the general procedure described above for displacement reaction (IPA, Conc. HCl) by using corresponding chloro compound 4 and amine 3. ¹H NMR (400 MHz, DMSO-d₆) δ: 9.69 (s, 1H), 8.50 (s, 1H), 8.25 (s, 1H), 8.10 (s, 1H), 7.89-7.75 (m, 3H), 7.47 (d, J=9.2 Hz, 1H), 6.78 (s, 1H), 4.44 (s, 3H), 4.13 (s, 3H), 3.90-3.72 (m, 8H); HPLC purity: 99.31%; LCMS Calculated for C₂₃H₂₃N₉O: 441.20; Observed: 442.00 (M+1).

N-(6-(1,3-dimethyl-1H-indazol-6-yl)-2-morpholinopyrimidin-4-yl)-2-methyl-2H-benzo[d][1,2,3]triazol-5-amine: The title compound has been synthesized by following the general procedure described above for displacement reaction (IPA, Conc. HCl) by using corresponding chloro compound 4 and amine 3. ¹H NMR (400 MHz, DMSO-d₆) δ: 9.66 (s, 1H), 8.48 (s, 1H), 8.17 (s, 1H), 7.88-7.70 (m, 3H), 7.51-7.43 (m, 1H), 6.78 (s, 1H), 4.44 (s, 3H), 4.04 (s, 3H), 3.90-3.76 (m, 8H), 2.52 (s,3H); HPLC purity: 98.68%; LCMS Calculated for C₂₄H₂₅N₉O: 455.22; Observed: 456.40 (M+1).

### Step 4: Synthesis of 1-methyl-1H-benzo[d][1,2,3]triazol-5-amine (6)

The title compound 6 has been synthesized by using the procedure described above for reduction for step 2 using the nitro compound 5 and 10% Pd—C. LCMS (m/z): 148.90 (M+1).

### Step 5

1-methyl-N-(6-(1-methyl-1H-indazol-6-yl)-2-morpholinopyrimidin-4-yl)-1H-benzo[d][1,2,3]triazol-5-amine: The title compound has been synthesized by following the General procedure for Buchwald Coupling described above using corresponding chloro compound 4 and amine 6. ¹H NMR (400 MHz, DMSO-d₆) δ: 9.66 (s, 1H), 8.47 (d, J=1.8 Hz, 1H), 8.25 (s, 1H), 8.10 (s, 1H), 7.89-7.74 (m, 3H), 7.73-7.66 (m, 1H), 6.75 (s, 1H), 4.28 (s, 3H), 4.13 (s, 3H), 3.89-3.75 (m, 8H); HPLC purity: 96.12%; LCMS Calculated for C₂₃H₂₃N₉O: 441.20; Observed: 442.00 (M+1).

N-(6-(1,3-dimethyl-1H-indazol-6-yl)-2-morpholinopyrimidin-4-yl)-1-methyl-1H-benzo[d][1,2,3]triazol-5-amine: The title compound has been synthesized by following the General procedure for Buchwald Coupling described above using corresponding chloro compound 4 and amine 6. ¹H NMR (400 MHz, DMSO-d₆) δ: 9.65 (s, 1H), 8.47 (d, J=1.7 Hz, 1H), 8.17 (s, 1H), 7.80 (dd, J=8.8, 2.8 Hz, 2H), 7.73 (d, J=8.4 Hz, 2H), 6.74 (s, 1H), 4.28 (s, 3H), 4.04 (s, 3H), 3.89-3.75 (m, 8H), 2.45 (s, 3H); HPLC purity: 99.35%; LCMS Calculated for C₂₄H₂₅N₉O: 455.22; Observed: 456.35 (M+1).

### Step 6: Synthesis of 1-methyl-1H-benzo[d][1,2,3]triazol-6-amine (8)

The title compound 8 has been synthesized by using the procedure described above for reduction for step 2 using the nitro compound 7 and 10% Pd—C. LCMS (m/z): 148.90 (M+1).

### Step 7

1-methyl-N-(6-(1-methyl-1H-indazol-6-yl)-2-morpholinopyrimidin-4-yl)-1H-benzo[d][1,2,3]triazol-6-amine: The title compound has been synthesized by following the General procedure for Buchwald Coupling described above using corresponding chloro compound 4 and amine 8. ¹H NMR (400 MHz, DMSO-d₆) δ: 9.87 (s, 1H), 8.57 (s, 1H), 8.26 (s, 1H), 8.10 (s, 1H), 7.94 (d, J=9.2 Hz, 1H), 7.86 (d, J=8.4 Hz, 1H), 7.81 (d, J=8.4 Hz, 1H), 7.36 (d, J=8.8 Hz, 1H),6.81 (s, 1H), 4.23 (s, 3H), 4.14 (s, 3H), 3.89-3.77 (m, 8H); HPLC purity: 98.33%; LCMS Calculated for C₂₃H₂₃N₉O: 441.20; Observed: 442.30 (M+1).

N-(6-(1,3-dimethyl-1H-indazol-6-yl)-2-morpholinopyrimidin-4-yl)-1-methyl-1H-benzo[d][1,2,3]triazol-6-amine: The title compound has been synthesized by following the General procedure for Buchwald Coupling described above using corresponding chloro compound 4 and amine 8. ¹H NMR (400 MHz, DMSO-d₆) δ: 9.86 (s, 1H), 8.56 (d, J=1.8 Hz, 1H), 8.18 (d, J=1.2 Hz, 1H), 7.94 (d, J=8.9 Hz, 1H), 7.84-7.71 (m, 2H), 7.36 (dd, J=9.0, 1.9 Hz, 1H), 6.80 (s, 1H), 4.23 (s, 3H), 4.04 (s, 3H), 3.93-3.77 (m, 8H), 2.52 (s, 3H); HPLC purity: 99.53%; LCMS Calculated for C₂₄H₂₅N₉O: 455.22; Observed: 456.25 (M+1).

### Examples 96-97

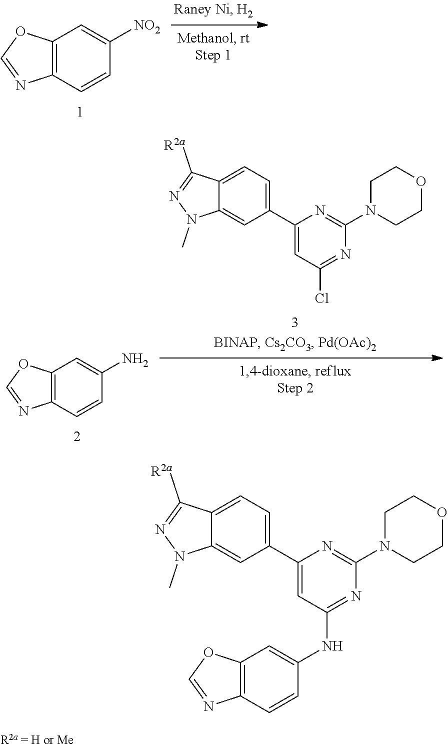

### Step 1: Synthesis of benzo[d]oxazol-6-amine (2)

To a stirred solution of compound 1 (0.6 g, 1 eq) in methanol (10 mL), Raney nickel (0.1 g) was added. The reaction mixture was stirred at room temperature under hydrogen atmosphere (balloon pressure) for 3 h. The progress of the reaction was monitored by TLC. After completion of the reaction, the reaction mixture was filtered through celite and evaporated under reduced pressure to afford the title compound 6. LCMS (m/z): 135.00 (M+1).

### Step 2

N-(6-(1-methyl-1H-indazol-6-yl)-2-morpholinopyrimidin-4-yl)benzo[d]oxazol-6-amine: The title compound has been synthesized by following the General procedure for Buchwald Coupling described above using corresponding chloro compound 3 and amine 2. ¹H NMR (400 MHz, DMSO-d₆) δ: 9.73 (s, 1H), 8.63 (s, 1H), 8.38 (d, J=1.9 Hz, 1H), 8.25 (s, 1H), 8.10 (s, 1H), 7.86 (d, J=8.5 Hz, 1H), 7.82-7.69 (m, 2H), 7.47 (dd, J=8.6, 2.0 Hz, 1H), 6.76 (s, 1H), 4.13 (s, 3H), 3.84-3.75 (m, 8H); HPLC purity: 96.64%; LCMS Calculated for C₂₃H₂₁N₇O₂: 427.18; Observed: 428.25 (M+1).

N-(6-(1,3-dimethyl-1H-indazol-6-yl)-2-morpholinopyrimidin-4-yl)benzo[d]oxazol-6-amine: The title compound has been synthesized by following the General procedure for Buchwald Coupling described above using corresponding chloro compound 3 and amine 2. ¹H NMR (400 MHz, DMSO-d₆) δ: 9.72 (s, 1H), 8.63 (d, J=1.0 Hz, 1H), 8.41-8.35 (m, 1H), 8.17 (s, 1H), 7.83-7.69 (m, 3H), 7.47 (dt, J=8.7, 1.4 Hz, 1H), 6.75 (s, 1H), 4.04 (s, 3H), 3.84-3.75 (m, 8H), 2.52 (s, 3H); HPLC purity: 96.36%; LCMS Calculated for C₂₄H₂₃N₇O₂: 441.19; Observed: 442.20 (M+1).

### Examples 98-101

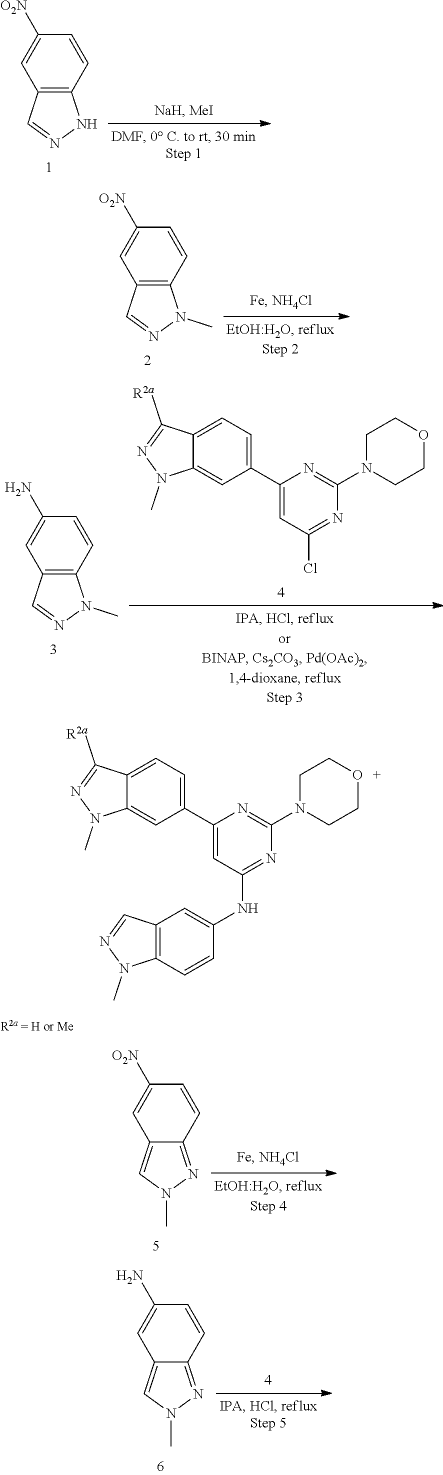

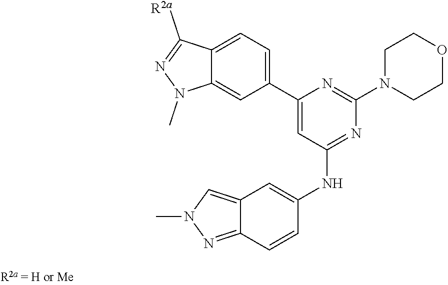

### Step 1: Synthesis of 1-methyl-5-nitro-1H-indazole (2) and 2-methyl-5-nitro-2H-indazole (5)

To a stirred solution of compound 1 (2 g, 1 eq) in DMF (20 mL), NaH (1.47 g, 3 eq) was added slowly at 0° C. followed by the addition of methyl iodide (2.3 mL, 3 eq) at same temperature. The reaction mixture was stirred at room temperature for 30 min. The progress of the reaction was monitored by TLC. After completion of the reaction, the reaction mixture was diluted with water and extracted with ethyl acetate (3×25 mL). Combined organic extracts were washed with brine, dried over anhydrous sodium sulfate and evaporated under reduced pressure. The crude product was purified by column chromatography on silica gel 100-200 mesh using 50% EtOAc-hexane to afford the title compounds 2 and 5 (confirmed by NOE). LCMS (m/z): 178.00 (M+1).

### Step 2: Synthesis of 1-methyl-1H-indazol-5-amine (3)

To a stirred solution of compound 2 (1 g, 1 eq) in ethanol (20 mL), iron powder (1.19 g, 4 eq), water (10 mL) and ammonium chloride (1.19 g, 4 eq) were added slowly. The reaction mixture was refluxed for 2 h. The progress of the reaction was monitored by TLC. After completion of the reaction, the reaction mixture was filtered through celite and evaporated under reduced pressure. The residue was diluted with water and extracted with ethyl acetate (2×25 mL). Combined organic extracts were dried over anhydrous sodium sulfate and evaporated under reduced pressure. The crude product was purified by column chromatography on silica gel 100-200 mesh using 60% EtOAc-hexane to afford the title compound 3. LCMS (m/z): 147.95 (M+1).

### Step 3

1-methyl-N-(6-(1-methyl-1H-indazol-6-yl)-2-morpholinopyrimidin-4-yl)-1H-indazol-5-amine: The title compound has been synthesized by following the general procedure described above for displacement reaction (IPA, Conc. HCl) by using corresponding chloro compound 4 and amine 3. ¹H NMR (400 MHz, DMSO-d₆) δ: 10.30 (s, 1H), 8.21(s,1H), 8.16 (s, 1H), 8.06 (d, J=5.6 Hz, 2H), 7.93 (d, J=8.4 Hz, 1H), 7.63 (dt, J=26.9, 8.7 Hz, 3H), 6.72 (s, 1H), 4.15 (s, 3H), 4.05 (s, 3H), 3.88-3.71 (m, 8H); HPLC purity: 99.76%; LCMS Calculated for C₂₄H₂₄N₈₀ (free base): 440.21; Observed: 441.30 (M+1).

N-(6-(1,3-dimethyl-1H-indazol-6-yl)-2-morpholinopyrimidin-4-yl)-1-methyl-1H-indazol-5-amine: The title compound has been synthesized by following the General procedure for Buchwald Coupling described above using corresponding chloro compound 4 and amine 3. ¹H NMR (400 MHz, MeOD) δ: 8.13 (d, J=1.3 Hz, 1H), 8.07 (d, J=1.9 Hz, 1H), 7.96 (d, J=1.0 Hz, 1H), 7.75 (d, J=1.1 Hz, 2H), 7.62 (dd, J=9.0, 1.9 Hz, 1H), 7.53 (d, J=9.0 Hz, 1H), 6.61 (s, 1H), 4.06 (s, 3H), 4.04 (s, 3H), 3.90-3.80 (m, 8H), 2.56 (s, 3H); HPLC purity: 98.07%; LCMS Calculated for C₂₅H₂₆N₈O: 454.22; Observed: 455.40 (M+1).

### Step 4: Synthesis of 2-methyl-2H-indazol-5-amine (6)

To a stirred solution of compound 5 (0.45 g, 1 eq) in ethanol:water (20 mL), iron powder (0.538 g, 4 eq) and ammonium chloride (0.538 g, 4 eq) were added slowly. The reaction mixture was refluxed for 2 h. The progress of the reaction was monitored by TLC. After completion of the reaction, the reaction mixture was filtered through celite and evaporated under reduced pressure. The residue was diluted with water and extracted with ethyl acetate (2×25 mL). Combined organic extracts were dried over anhydrous sodium sulfate and evaporated under reduced pressure to afford the title compound 6. LCMS (m/z): 148.00 (M+1).

### Step 5

2-methyl-N-(6-(1-methyl-1H-indazol-6-yl)-2-morpholinopyrimidin-4-yl)-2H-indazol-5-amine: The title compound has been synthesized by following the general procedure described above for displacement reaction (IPA, Conc. HCl) by using corresponding chloro compound 4 and amine 6. ¹H NMR (400 MHz, DMSO-d₆) δ: 10.60 (s,1H),8.34 (s, 1H), 8.24-8.15 (m, 2H), 8.08 (s, 1H), 7.94 (d, J=8.4 Hz, 1H), 7.59 (dd, J=26.4, 8.8 Hz, 2H), 7.43 (d, J=9.3 Hz, 1H), 6.75 (s, 1H), 4.16 (s, 3H), 4.15 (s, 3H), 3.85-3.76 (m, 8H); HPLC purity: 99.31%; LCMS Calculated for C₂₄H₂₄N₈O (free base): 440.21; Observed: 441.30 (M+1).

N-(6-(1,3-dimethyl-1H-indazol-6-yl)-2-morpholinopyrimidin-4-yl)-2-methyl-2H-indazol-5-amine: The title compound has been synthesized by following the general procedure described above for displacement reaction (IPA, Conc. HCl) by using corresponding chloro compound 4 and amine 6. ¹H NMR (400 MHz, MeOD) δ: 8.06-7.96 (m, 3H), 7.65 (d, J=1.1 Hz, 2H), 7.46 (d, J=9.1 Hz, 1H), 7.32 (dd, J=9.2, 2.0 Hz, 1H), 6.53 (s, 1H), 4.09 (s, 3H), 3.95 (s, 3H), 3.81-3.71 (m, 8H), 2.44 (s, 3H); HPLC purity: 99.91%; LCMS Calculated for C₂₅H₂₆N₈O: 454.22; Observed: 455.40 (M+1).

### Example 102-105

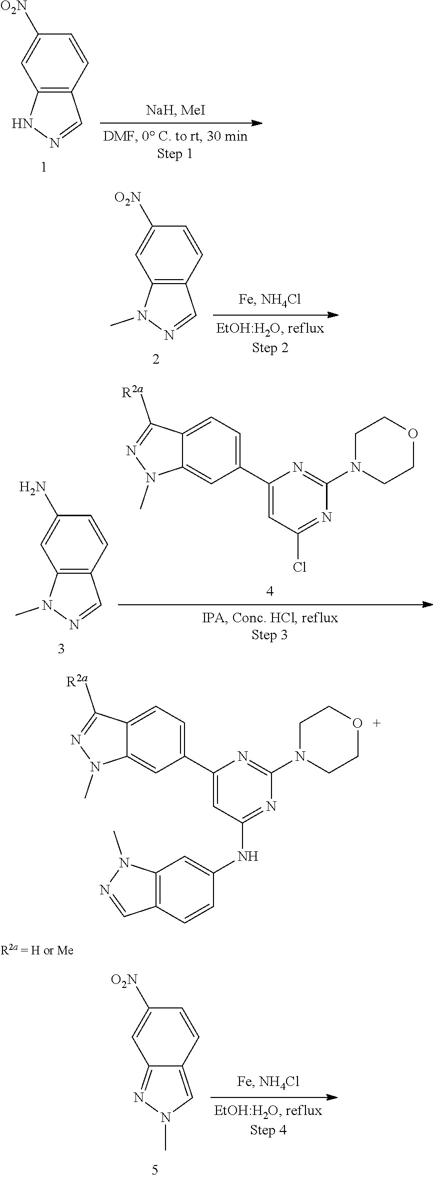

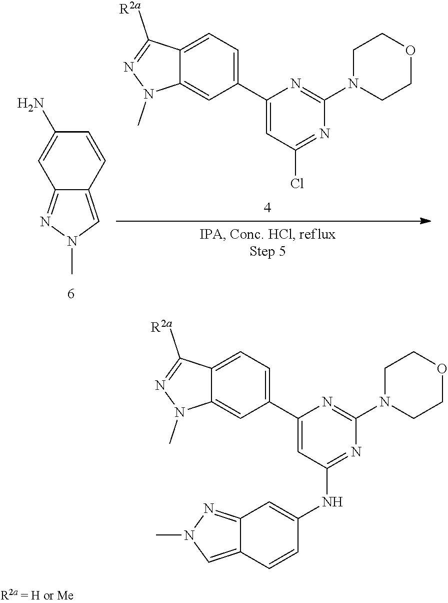

### Step 1: Synthesis of 1-methyl-6-nitro-1H-indazole (2) and 2-methyl-6-nitro-2H-indazole (5)

To a stirred solution of 1 (1 g, 1 eq) in DMF (10 mL), NaH (0.29 g, 2 eq) was added at 0° C. and stirred for 15 min followed by the addition of methyl iodide (1.3 g, 1.5 eq). The reaction mixture was stirred at room temperature for 3 h. The progress of the reaction was monitored by TLC. After completion of the reaction, the reaction mixture was diluted with water and extracted with ethyl acetate (2×30 mL). Combined organic extracts were washed with brine, dried over anhydrous sodium sulfate and evaporated under reduced pressure to the afford title compounds 2 and 3. # 2: ¹H NMR (400 MHz, CDC13) δ: 8.38 (s, 1H), 8.10 (s, 1H), 8.01 (dd, J=8.8, 2.0 Hz, 1H), 7.83 (d, J=8.8 Hz, 1H). # 3: ¹H NMR (400 MHz, CDCl₃) δ: 8. 67 (s, 1H), 8.02 (s, 1H), 7.88 (dd, J=9.2, 2.0 Hz, 1H), 7.74 (d, J=9.2 Hz, 1H).

### Step 2: Synthesis of 1-methyl-1H-indazol-6-amine (3)

The title compound has been synthesized by following the general procedure described above for reduction using the nitro compound 2 and Fe/NH₄Cl. LCMS (m/z): 148.00 (M+1).

### Step 3

1-methyl-N-(6-(1-methyl-1H-indazol-6-yl)-2-morpholinopyrimidin-4-yl)-1H-indazol-6-amine: The title compound has been synthesized by following the general procedure described above for displacement reaction (IPA, Conc. HCl) by using corresponding chloro compound 4 and amine 3. ¹H NMR (400 MHz, DMSO-d₆) δ: 9.69 (s, 1H), 8.40 (s, 1H), 8.25 (d, J=1.3 Hz, 1H), 8.10 (d, J=1.0 Hz, 1H), 7.91 (s, 1H), 7.85 (d, J=8.4 Hz, 1H), 7.80-7.77 (m, 1H), 7.66 (d, J=8.7 Hz, 1H), 7.12 (dd, J=8.7, 1.7 Hz, 1H), 6.79 (s, 1H), 4.14 (s, 3H), 3.97 (s, 3H), 3.89-3.77 (m, 8H); HPLC purity: 99.73%; LCMS Calculated for C₂₄H₂₄N₈O: 440.21; Observed: 441.35 (M+1).

N-(6-(1,3-dimethyl-1H-indazol-6-yl)-2-morpholinopyrimidin-4-yl)-1-methyl-1H-indazol-6-amine : The title compound has been synthesized by following the general procedure described above for displacement reaction (IPA, Conc. HCl) by using corresponding chloro compound 4 and amine 3. ¹H NMR (400 MHz, DMSO-d₆) δ: 10.09 (s, 1H), 8.33 (s, 1H), 8.16 (s, 1H), 7.96 (s, 1H), 7.85 (d, J=8.3 Hz, 1H), 7.67 (dd, J=23.8, 8.6 Hz, 2H), 7.17 (d, J=8.6 Hz, 1H), 6.80 (s, 1H), 4.05 (s, 3H), 3.99 (s, 3H), 3.90-3.78 (m, 8H), 2.52 (s, 3H); HPLC purity: 97.96%; LCMS Calculated for C₂₅H₂₆N₈O: 454.22; Observed: 455.40 (M+1).

### Step 4: Synthesis of 2-methyl-2H-indazol-6-amine (6)

The title compound has been synthesized by following the general procedure described above for reduction using the nitro compound 5 and Fe/NH₄Cl. LCMS (m/z): 148.00 (M+1).

### Step 5

2-methyl-N-(6-(1-methyl-1H-indazol-6-yl)-2-morpholinopyrimidin-4-yl)-2H-indazol-6-amine: The title compound has been synthesized by following the general procedure described above for displacement reaction (IPA, Conc. HCl) by using corresponding chloro compound 4 and amine 6. ¹H NMR (400 MHz, DMSO-d₆) δ: 10.85 (s, 1H), 8.40 (s, 1H), 8.26-8.15 (m, 3H), 7.94 (d, J=8.4 Hz, 1H), 7.75 (d, J=8.8 Hz, 1H), 7.58 (d, J=8.4 Hz, 1H), 7.29 (dd, J=8.9, 1.8 Hz, 1H), 6.90 (s, 1H), 4.17 (s, 3H), 4.15 (s, 3H), 3.90-3.78 (m, 8H); HPLC purity: 95.03%; LCMS Calculated for C₂₄H₂₄N₈O: 440.21; Observed: 441.30 (M+1).

N-(6-(1,3-dimethyl-1H-indazol-6-yl)-2-morpholinopyrimidin-4-yl)-2-methyl-2H-indazol-6-amine: The title compound has been synthesized by following the general procedure described above for displacement reaction (IPA, Conc. HCl) by using corresponding chloro compound 4 and amine 6. ¹H NMR (400 MHz, DMSO-d₆) δ: 10.97 (s, 1H), 8.39 (s, 1H), 8.17 (d, J=15.7 Hz, 2H), 7.88 (d, J=8.4 Hz, 1H), 7.74 (d, J=8.9 Hz, 1H), 7.52 (d, J=8.5 Hz, 1H), 7.27 (d, J=9.3 Hz, 1H), 6.87 (s, 1H), 4.17 (s, 3H), 4.06 (s, 3H), 3.89-3.78 (m, 8H), 2.53 (s, 3H); HPLC purity: 98.89%; LCMS Calculated for C₂₅H₂₆N₈O: 454.22; Observed: 455.30 (M+1).

### Example 106-107

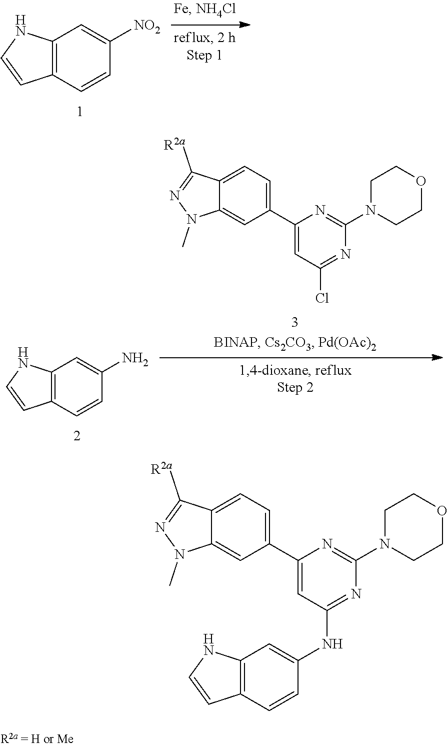

### Step 1: Synthesis of 1H-indol-6-amine (2)

The title compound (crude) has been synthesized by using the general procedure described above for reduction using the nitro compound 1 and Fe/NH₄Cl. LCMS (m/z): 132.95 (M+1).

### Step 2

N-(6-(1-methyl-1H-indazol-6-yl)-2-morpholinopyrimidin-4-yl)-1H-indol-6-amine: The title compound has been synthesized by following the General procedure for Buchwald Coupling described above using corresponding chloro compound 3 and amine 2. ‘H NMR (400 MHz, DMSO-d₆) δ: 11.02 (s, 1H), 9.31 (s, 1H), 8.22 (s, 1H), 8.09 (s, 1H), 8.03 (s, 1H), 7.84 (d, J=8.5 Hz, 1H), 7.74 (d, J=8.6 Hz, 1H), 7.45 (d, J=8.4 Hz, 1H), 7.26 (t, J=2.7 Hz, 1H), 7.09 (dd, J=8.4, 1.9 Hz, 1H), 6.71 (s, 1H), 6.36 (t, J=2.4 Hz, 1H), 4.12 (s, 3H), 3.84-3.74 (m, 8H); HPLC purity: 94.31%; LCMS Calculated for C₂₄H₂₃N₇O: 425.20; Observed: 426.30 (M+1).

N-(6-(1,3-dimethyl-1H-indazol-6-yl)-2-morpholinopyrimidin-4-yl)-1H-indol-6-amine: The title compound has been synthesized by following the General procedure for Buchwald Coupling described above using corresponding chloro compound 3 and amine 2. ¹H NMR (400 MHz, DMSO-d₆) δ: 11.02 (s, 1H), 9.31 (s, 1H), 8.09 (s, 1H), 8.03 (s, 1H), 7.84 (d, J=8.5 Hz, 1H), 7.74 (d, J=8.6 Hz, 1H), 7.45 (d, J=8.4 Hz, 1H), 7.26 (t, J=2.7 Hz, 1H), 7.09 (dd, J=8.4, 1.9 Hz, 1H), 6.71 (s, 1H), 6.36 (t, J=2.4 Hz, 1H), 4.12 (s, 3H), 3.83-3.74 (m, 8H), 2.50 (s, 3H); HPLC purity: 92.29%; LCMS Calculated for C₂₅H₂₅N₇O: 439.21; Observed: 440.35 (M+1).

### Examples 108-109

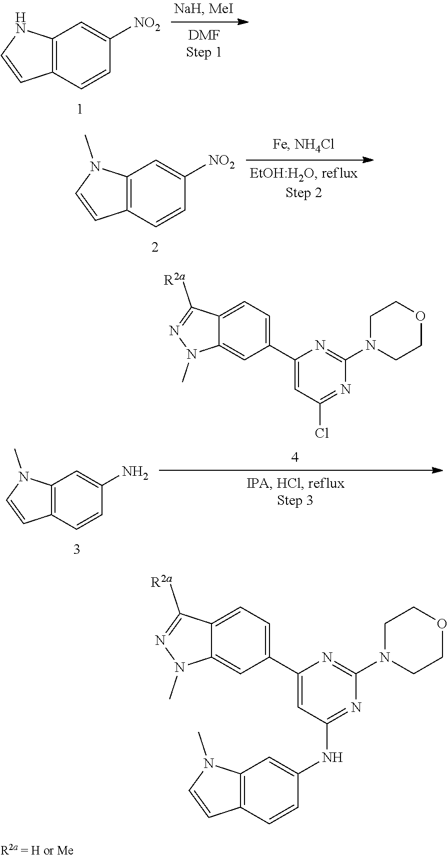

### Step 1: Synthesis of 1-methyl-6-nitro-1H-indole (2)

To a stirred solution of compound 1 (1 g, 1 eq) in DMF (10 mL), NaH (0.221 g,1.5 eq) was added at 0° C. followed by the addition of methyl iodide (2.3 mL, 3 eq) at same temperature. The reaction mixture was stirred at same temperature for 30 min. The progress of the reaction was monitored by TLC. After completion of the reaction, the reaction mixture was diluted with water and extracted with ethyl acetate (3×25 mL). Combined organic extracts were washed with brine, dried over anhydrous sodium sulfate and evaporated under reduced pressure. The crude product was purified by column chromatography on silica gel 100-200 mesh using 20% EtOAc-hexane to afford the title compound 2.

### Step 2: Synthesis of 1-methyl-1H-indol-6-amine (3)

The title compound (crude) has been synthesized by following the general procedure described above for reduction using the nitro compound 2 and Fe/NH₄Cl. LCMS (m/z): 147.00 (M+1).

### Step 3

1-methyl-N-(6-(1-methyl-1H-indazol-6-yl)-2-morpholinopyrimidin-4-yl)-1H-indol-6-amine: The title compound has been synthesized by following the general procedure described above for displacement reaction (IPA, Conc. HCl) by using corresponding chloro compound 4 and amine 3. ¹H NMR (400 MHz, DMSO-d₆) δ: 9.41 (s, 1H), 8.26-8.16 (m, 2H), 8.09 (d, J=1.0 Hz, 1H), 7.84 (d, J=8.5 Hz, 1H), 7.76 (dd, J=8.5, 1.4 Hz, 1H), 7.46 (d, J=8.4 Hz, 1H), 7.24 (d, J=3.1 Hz, 1H), 7.04 (dd, J=8.5, 1.8 Hz, 1H), 6.72 (s, 1H), 6.38-6.32 (m, 1H), 4.13 (s, 3H), 3.91-3.73 (m, 8H), 3.75 (s, 3H); HPLC purity: 95.89%; LCMS Calculated for C₂₅H₂₅N₇O: 439.21; Observed: 440.35 (M+1).

N-(6-(1,3-dimethyl-1H-indazol-6-yl)-2-morpholinopyrimidin-4-yl)-1-methyl-1H-indol-6-amine: The title compound has been synthesized by following the general procedure described above for displacement reaction (IPA, Conc. HCl) by using corresponding chloro compound 4 and amine 3. ¹H NMR (400 MHz, DMSO-d₆) δ: 9.41 (s, 1H), 8.19 (s, 1H), 8.15 (s, 1H), 7.78 (d, J=8.0 Hz, 1H), 7.70 (d, J=8.4 Hz, 1H), 7.46 (d, J=8.4, 1H), 7.23 (d, J=7.2 Hz, 1H), 7.04 (dd, J=8.5, 1.6Hz, 1H), 6.71 (s, 1H), 6.35-6.34 (m, 1H), 4.13 (s, 3H), 3.91-3.75 (m, 8H), 3.73 (s, 3H), 2.51 (s, 3H); HPLC purity: 95.09%; LCMS Calculated for C₂₆H₂₇N₇O: 453.23; Observed: 454.30 (M+1).

### Example 110-111

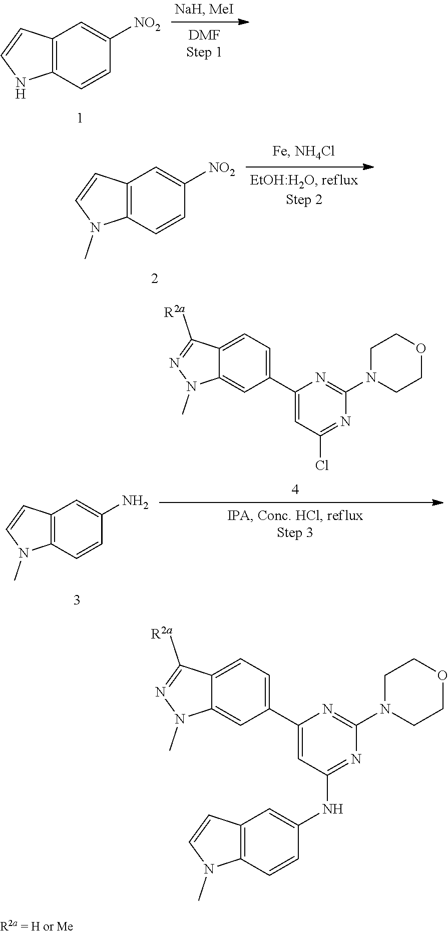

### Step 1: Synthesis of 1-methyl-5-nitro-1H-indole (2)

To a stirred solution of compound 1 (1 g, 1 eq) in DMF (5 mL), NaH (0.222 g, 1.5 eq) was added at 0° C. followed by the addition of methyl iodide (1.3 mL, 1.5 eq) at same temperature. The reaction mixture was stirred at same temperature for 1 h. The progress of the reaction was monitored by TLC. After completion of the reaction, the reaction mixture was diluted with water and extracted with ethyl acetate (2×25 mL). Combined organic extracts were washed with brine, dried over anhydrous sodium sulfate and evaporated under reduced pressure to afford the title compound 2.

### Step 2: Synthesis of 1-methyl-1H-indol-5-amine (3)

The title compound has been synthesized by following the general procedure described above for reduction using the nitro compound 2 and Fe/NH₄Cl. LCMS (m/z): 147.00 (M+1).

### Step 3

1-methyl-N-(6-(1-methyl-1H-indazol-6-yl)-2-morpholinopyrimidin-4-yl)-1H-indol-5-amine: The title compound has been synthesized by following the general procedure described above for displacement reaction (IPA, Conc. HCl) by using corresponding chloro compound 4 and amine 3. ¹H NMR (400 MHz, DMSO-d₆) δ: 9.41 (s, 1H), 8.26-8.16 (m, 2H), 8.09 (d, J=1.0 Hz, 1H), 7.84 (d, J=8.5 Hz, 1H), 7.76 (dd, J=8.5, 1.4 Hz, 1H), 7.46 (d, J=8.4 Hz, 1H), 7.24 (d, J=3.1 Hz, 1H), 7.04 (dd, J=8.5, 1.8 Hz, 1H), 6.72 (s, 1H), 6.38-6.32 (m, 1H), 4.13 (s, 3H), 3.91-3.73 (m, 8H), 3.75 (s, 3H); HPLC purity: 95.89%; LCMS Calculated for C₂₅H₂₅N₇O: 439.21; Observed: 440.35 (M+1).

N-(6-(1,3-dimethyl-1H-indazol-6-yl)-2-morpholinopyrimidin-4-yl)-1-methyl-1H-indol-5-amine: The title compound has been synthesized by following the general procedure described above for displacement reaction (IPA, Conc. HCl) by using corresponding chloro compound 4 and amine 3. ¹H NMR (400 MHz, DMSO-d₆) δ: 9.18 (s, 1H), 8.13 (s, 1H), 7.84-7.73 (m, 2H), 7.67 (d, J=8.6 Hz, 1H), 7.39 (d, J=2.8 Hz, 2H), 7.29 (d, J=3.0 Hz, 1H), 6.63 (s, 1H), 6.38 (d, J=3.0 Hz, 1H), 4.02 (s, 3H), 3.80-3.71 (m, 8H), 3.71 (s, 3H), 2.50 (s, 3H); HPLC purity: 96.12%; LCMS Calculated for C₂₆H₂₇N₇O: 453.23; Observed: 454.35 (M+1).

### Examples 112-113

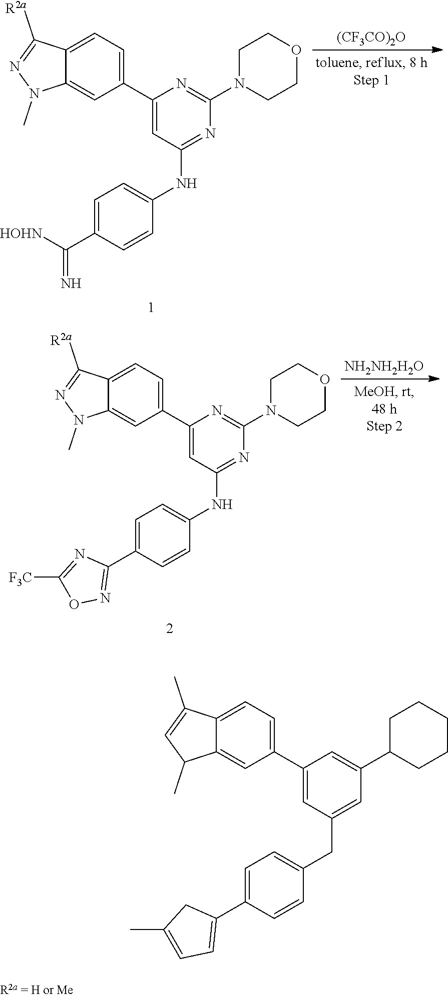

### Step 1: General procedure for the synthesis of compound 2

To a stirred solution of compound 1 (1 eq) in toluene, trifluoroacetic anhydride (1.1 eq) was added and heated to reflux for 8 h. The progress of the reaction was monitored by TLC. After completion of the reaction, the reaction mixture was evaporated to dryness. The residue was diluted with water and extracted with ethyl acetate. Combined organic extracts were washed with brine, dried over anhydrous sodium sulfate and evaporated under reduced pressure. The crude product was purified by column chromatography on silica gel 100-200 mesh to afford the title compound 2.

| Structure 2 | LCMS/¹H NMR |
|---|---|
| 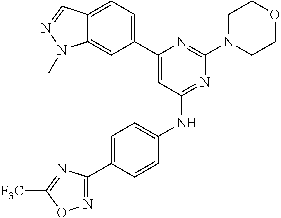 [SMILES: Cn1ncc2ccc(-c3cc(Nc4ccc(-c5noc(C(F)(F)F)n5)cc4)nc(N4CCOCC4)n3)cc21] | ¹H NMR (400 MHz, DMSO-d₆) δ: 10.21 (s, 1H), 8.25 (d, J = 1.3 Hz, 1H), 8.13 (d, J = 1.0 Hz, 1H), 8.08-8.01 (m, 2H), 7.96-7.86 (m, 3H), 7.72 (d, J = 8.6 Hz, 1H), 6.81 (s, 1H), 4.14 (s, 3H), 3.85-3.76 (m, 8H); HPLC purity: 91.63%; LCMS Calculated for C₂₅H₂₁F₃N₈O₂: 521.17; Observed: |
| | 522.30 (M + 1). |
| 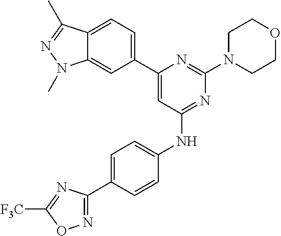 [SMILES: Cc1nn(C)c2cc(-c3cc(Nc4ccc(-c5noc(C(F)(F)F)n5)cc4)nc(N4CCOCC4)n3)ccc12] | LCMS (m/z): 537.30 (M + 1) |

### Step 2

6-(1-methyl-1H-indazol-6-yl)-2-morphohno-N-(4-(5-(trifluoromethyl)-4H-1,2,4-triazol-3-yl)phenyl)pyrimidin-4-amine: To a stirred solution of compound 2 where R is H (0.04 g, 1 eq) in methanol (2 mL), hydrazine hydrate (0.2 mL) was added and stirred at room temperature for 18 h. The progress of the reaction was monitored by TLC. After completion of the reaction, the reaction mixture was evaporated to dryness. The residue was diluted with water and extracted with ethyl acetate (2×10 mL). Combined organic extracts were washed with brine, dried over anhydrous sodium sulfate and evaporated under reduced pressure. The crude product was purified by preparative HPLC to afford the title compound. ¹H NMR (400 MHz, DMSO-d₆) δ: 15.20 (s, 1H), 10.21 (s, 1H), 8.25 (d, J=1.3 Hz, 1H), 8.13 (d, J=1.0 Hz, 1H), 8.08-8.01 (m, 2H), 7.96-7.86 (m, 3H), 7.72 (d, J=8.6 Hz, 1H), 6.81 (s, 1H), 4.14 (s, 3H), 3.85-3.76 (m, 8H); HPLC purity: 97.45%; LCMS Calculated for C₂₅H₂₂F₃N₉O: 521.19; Observed: 522.30 (M+1).

6-(1,3-dimethyl-1H-1-indazol-6-yl)-2-morphohno-N-(4-(5-(trifluoromethyl)-4H-1,2,4-triazol-3-yl)phenyl)pyrimidin-4-amine: The title compound has been synthesized by following the procedure described above for 6-(1-methyl-1H-indazol-6-yl)-2-morpholino-N-(4-(5-(trifluoromethyl)-4H-1,2,4-triazol-3-yl)phenyl)pyrimidin-4-amine using corresponding compound 2 where R is methyl and hydrazine hydrate. ¹H NMR (400 MHz, DMSO-d₆) δ: 15.14 (s, 1H), 10.02 (s, 1H), 8.17 (s, 1H), 8.03 (d, J=8.4 Hz, 2H), 7.91 (d, J=8.5 Hz, 2H), 7.82 (d, J=8.4 Hz, 1H), 7.70 (d, J=8.5 Hz, 1H), 6.78 (s, 1H), 4.04 (s, 3H), 3.84-3.76 (m, 8H), 2.49 (s, 3H); HPLC purity: 96.2%; LCMS Calculated for C₂₆H₂₄F₃N₉O: 535.21; Observed: 536.25 (M+1).

### Example 114

### Synthesis of N-(4-(1H-pyrrol-2-yl)phenyl)-6-(1-methyl-1H-indazol-6-yl)-2-morpholinopyrimidin-4-amine

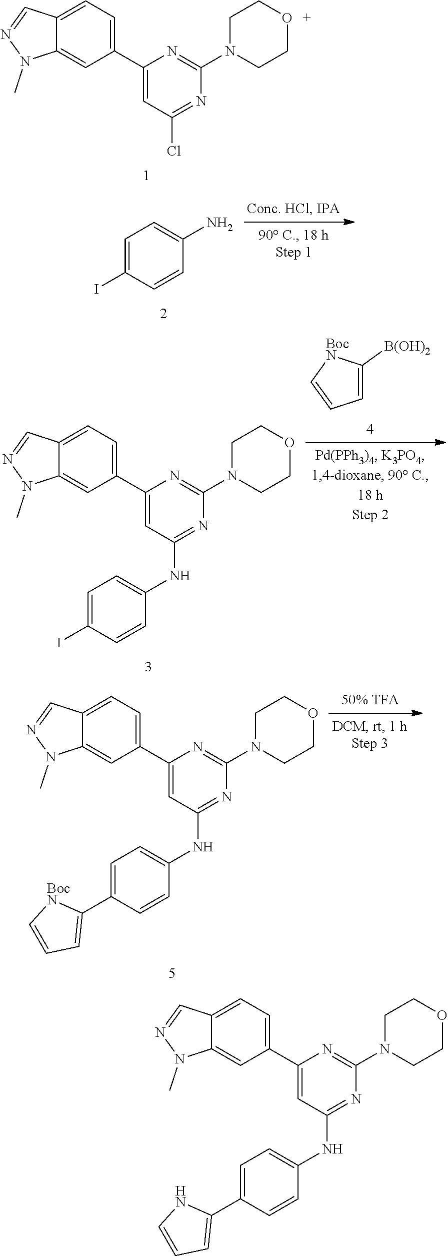 [SMILES: Cn1ncc2ccc(-c3cc(Cl)nc(N4CCOCC4)n3)cc21 ; Nc1ccc(I)cc1 ; CC(C)(C)OC(=O)n1cccc1B(O)O ; Cn1ncc2ccc(-c3cc(Nc4ccc(I)cc4)nc(N4CCOCC4)n3)cc21 ; Cn1ncc2ccc(-c3cc(Nc4ccc(-c5cccn5C(=O)OC(C)(C)C)cc4)nc(N4CCOCC4)n3)cc21 ; Cn1ncc2ccc(-c3cc(Nc4ccc(-c5ccc[nH]5)cc4)nc(N4CCOCC4)n3)cc21]

### Step 1: Synthesis of N-(4-iodophenyl)-6-(1-methyl-1H-indazol-6-yl)-2-morpholinopyrimidin-4-amine (3)

The title compound has been synthesized by following the general procedure described above for displacement reaction (IPA, Conc. HCl) by using chloro compound 1 and amine 2. LCMS (m/z): 513.20 (M+1).

### Step 2: Synthesis of tert-butyl 2-(4-46-(1-methyl-1H-indazol-6-yl)-2-morpholinopyrimidin-4-yl)amino) phenyl)-1H-pyrrole-1-carboxylate (5)

The title compound has been synthesized by following the General Procedure for Suzuki Coupling described above using compound 3 and Boronic acid 4. LCMS (m/z): 552.40 (M+1).

### Step 3: Synthesis of N-(4-(1H-pyrrol-2-yl)phenyl)-6-(1-methyl-1H-indazol-6-yl)-2-morpholinopyrimidin-4-amine

To a stirred solution of compound 5 (0.08 g, 1 eq) in dichloromethane (2 mL), TFA (2 mL) was added and stirred at room temperature for 1 h. The progress of the reaction was monitored by TLC. After completion of the reaction, the reaction mixture was evaporated under reduced pressure. The residue was neutralized with saturated sodium bicarbonate solution and extracted with ethyl acetate (2×20 mL). Combined organic extracts were dried over anhydrous sodium sulfate and evaporated under reduced pressure. The crude product was purified by preparative HPLC to afford the title compound. ¹H NMR (400 MHz, MeOD) δ: 8.20 (s, 1H), 8.03 (s, 1H), 7.86-7.73 (m, 2H), 7.63 (d, J=8.7 Hz, 2H), 7.57-7.50 (m, 2H), 6.78 (q, J=2.2 Hz, 1H), 6.63 (s, 1H), 6.41 (d, J=3.3 Hz, 1H), 6.15 (q, J=2.7 Hz, 1H), 4.14 (s, 3H), 3.96-3.81 (m, 8H); HPLC purity: 98.91%; LCMS Calculated for C₂₆H₂₅N₇O: 451.21; Observed: 452.25 (M+1).

### Example 115-116

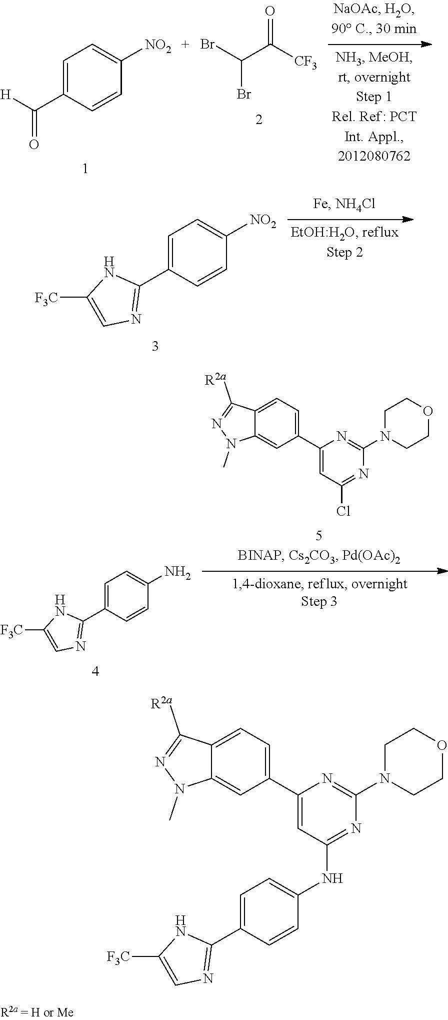

### Step 1: Synthesis of 2-(4-nitrophenyl)-5-(trifluoromethyl)-1H-imidazole (3)

To a stirred solution of sodium acetate (0.54g, 2 eq) in water (10 mL),compound 2(0.98 g, 1.1 eq) was added and stirred at 90° C. for 30 min. The reaction mixture was allowed to cool to room temperature, compound 1(0.5 g, 1 eq), aqueous ammonia (10 mL) and methanol (20 mL) were added. The reaction mixture was stirred at room temperature for overnight. The progress of the reaction was monitored by TLC. After completion of the reaction, reaction mixture was evaporated under reduced pressure. The residue was dissolved in water (50 mL) and extracted with ethyl acetate (2×25 mL). Combined organic extracts were dried over anhydrous sodium sulfate and evaporated under reduced pressure. The crude product was purified by column chromatography on silica gel 100-200 mesh using 20% EtOAc-hexane to afford the title compound 3. LCMS (m/z): 258 (M+1).

### Step 2: Synthesis of 4-(5-(trifluoromethyl)-1H-imidazol-2-yl)aniline(4)

To a stirred solution of compound 3(0.5 g, 1 eq) in ethanol (10 mL), iron powder (0.544 g, 5 eq), water (5 mL) and ammonium chloride (0.515 g, 5 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (50 mL), washed with brine, dried over anhydrous sodium sulfate and evaporated under reduced pressure to afford the title compound 4. LCMS (m/z): 228 (M+1).

### Step 3

6-(1-methyl-1H-indazol-6-yl)-2-morpholino-N-(4-(5-(trifluoromethyl)-1H-imidazol-2-yl)phenyl)pyrimidin-4-amine: The title compound has been synthesized by following the general procedure described above for Buchwald Coupling by using corresponding chloro compound 5 and amine compound 4. ¹H NMR (400 MHz, DMSO-d₆)δ: 13.00 (s, 1H), 9.66 (s, 1H), 8.25 (s, 1H), 8.10 (s, 1H), 8.04 (s, 2H), 7.94 (d, J=8.4 Hz, 2H), 7.92-7.75 (m, 5H), 6.76 (s, 1H), 4.14 (s, 3H), 3.87-3.80 (m, 4H), 3.78-3.70 (m, 4H); HPLC purity: 98.06%; LCMS Calculated for C₂₆H₂₃F₃N₈O: 520.19; Observed: 521.30 (M+1).

6-(1,3-dimethyl-1H-indazol-6-yl)-2-morpholino-N-(4-(5-(trifluoromethyl)-1H-imidazol-2-yl)phenyl)pyrimidin-4-amine: The title compound has been synthesized by following the general procedure described above for Buchwald coupling by using corresponding chloro compound 5 and amine compound 4. ¹H NMR (400 MHz, DMSO-d₆) δ: 12.99 (s, 1H), 9.64 (s, 1H), 8.17 (s, 1H), 8.02-7.67 (m, 7H), 6.74 (s, 1H), 4.04 (s, 3H), 3.79 (dt, J=35.9, 4.6 Hz, 8H), 2.49 (s, 3H); HPLC purity: 98.8%; LCMS Calculated for C₂₇H₂₅F₃N₈O (free base): 534.21; Observed: 535.25 (M+1).

### Example 117

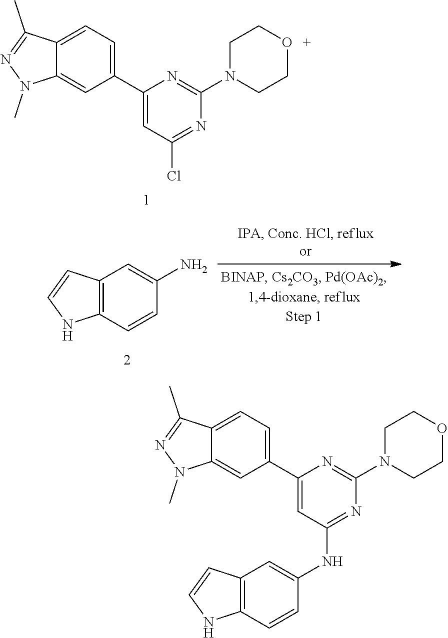 [SMILES: Cc1nn(C)c2cc(-c3cc(Cl)nc(N4CCOCC4)n3)ccc12 ; Nc1ccc2[nH]ccc2c1 ; Cc1nn(C)c2cc(-c3cc(Nc4ccc5[nH]ccc5c4)nc(N4CCOCC4)n3)ccc12]

N-(6-(1,3-dimethyl-1H-indazol-6-yl)-2-morpholinopyrimidin-4-yl)-1H-indol-5-amine: The title compound has been synthesized by following the general procedure described above for displacement reaction (IPA, Conc. HCl) by using corresponding chloro compound 1 and amine 2. ¹H NMR (400 MHz, DMSO-d₆) δ: 11.16 (s, 1H), 8.11 (s, 1H), 7.87 (d, J=9.6 Hz, 1H), 7.50 (s, 1H), 7.45-7.34 (m, 2H), 7.31 (s, 1H), 6.65 (s, 1H), 6.44 (t, J=2.4 Hz, 1H), 4.05 (s, 3H),3.83-3.74 (m, 8H),2.50 (s, 3H); HPLC purity: 99.64%; LCMS Calculated for C₂₄H₂₃N₇O: 439.21; Observed: 440.35 (M+1).

### Example 118

### Synthesis of N-(4-(1H-pyrrol-2-yl)phenyl)-6-(1,3-dimethyl-1H-indazol-6-yl)-2-morpholinopyrimidin-4-amine

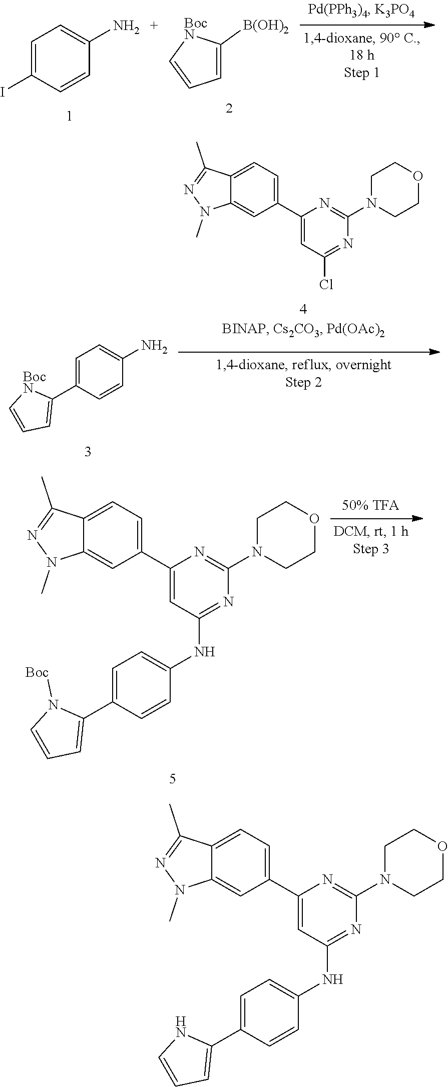 [SMILES: Nc1ccc(I)cc1 ; CC(C)(C)OC(=O)n1cccc1B(O)O ; Cc1nn(C)c2cc(-c3cc(Cl)nc(N4CCOCC4)n3)ccc12 ; CC(C)(C)OC(=O)n1cccc1-c1ccc(N)cc1 ; Cc1nn(C)c2cc(-c3cc(Nc4ccc(-c5cccn5C(=O)OC(C)(C)C)cc4)nc(N4CCOCC4)n3)ccc12 ; Cc1nn(C)c2cc(-c3cc(Nc4ccc(-c5ccc[nH]5)cc4)nc(N4CCOCC4)n3)ccc12]

### Step 1: Synthesis of tert-butyl 2-(4-aminophenyl)-1H-pyrrole-1-carboxylate (3)

The title compound has been synthesized by following the General Procedure for Suzuki Coupling described above using compound 1 and Boronic acid 2. LCMS (m/z): 259.00 (M+1).

### Step 2: Synthesis of tert-butyl 2-(4-((6-(1,3-dimethyl-1H-indazol-6-yl)-2-morpholinopyrimidin-4-yl) amino)phenyl)-1H-pyrrole-1-carboxylate (5)

The title compound has been synthesized by following the General procedure for Buchwald Coupling described above using chloro compound 4 and amine 3. LCMS (m/z): 566.50 (M+1).

### Step 3: Synthesis of N-(4-(1H-pyrrol-2-yl)phenyl)-6-(1,3-dimethyl-1H-indazol-6-yl)-2-morpholino pyrimidin-4-amine

To a stirred solution of compound 5 (0.08 g, 1 eq) in dichloromethane (2 mL), TFA (2 mL) was added and stirred at room temperature for 1 h. The progress of the reaction was monitored by TLC. After completion of the reaction, the reaction mixture was evaporated under reduced pressure. The residue was neutralized with saturated sodium bicarbonate solution and extracted with ethyl acetate (2×20 mL). Combined organic extracts were dried over anhydrous sodium sulfate and evaporated under reduced pressure. The crude product was purified by column chromatography on silica gel 100-200 mesh using 60% EtOAc-hexane to afford the title compound. ¹H NMR (400 MHz, DMSO-d₆) δ: 11.14 (s, 1H), 9.42 (s, 1H), 8.15 (s, 1H), 7.79 (d, J=8.5 Hz, 1H), 7.75-7.64 (m, 3H), 7.58 (d, J=8.5 Hz, 2H), 6.79 (s, 1H), 6.70 (s, 1H), 6.42 (s, 1H), 6.09 (q, J=2.8 Hz, 1H), 4.03 (s, 3H), 3.82-3.74 (m, 8H), 2.58 (s, 3H); HPLC purity: 95.22%; LCMS Calculated for C₂₇H₂₇N₇O: 465.23; Observed: 466.25 (M+1).

### Examples 119-120

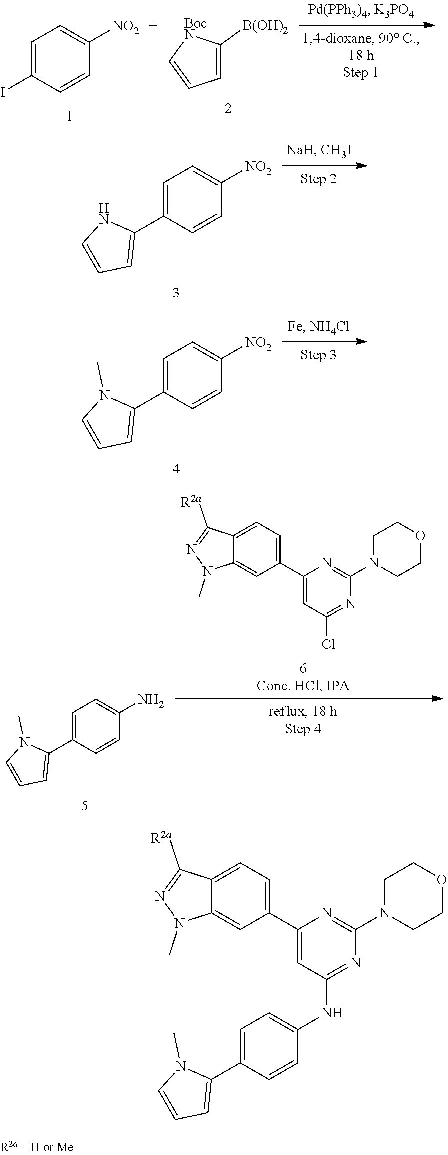

### Step 1: Synthesis of 2-(4-nitrophenyl)-1H-pyrrole (3)

The title compound has been synthesized by following the General Procedure for Suzuki Coupling described above using compound 1 and Boronic acid 2. LCMS (m/z): 259.00 (M+1).

### Step 2: Synthesis of 1-methyl-2-(4-nitrophenyl)-1H-pyrrole (4)

To a stirred solution of compound 3 (0.8 g, 1 eq) in DMF (15 mL), NaH (0.153 g, 1.5 eq) was added at 0° C. and stirred for 30 min followed by the addition of methyl iodide (0.906 g, 1.5 eq) at same temperature. The reaction mixture was stirred at same temperature for 1 h. The progress of the reaction was monitored by TLC. After completion of the reaction, the reaction mixture was diluted with water and extracted with ethyl acetate (3×25 mL). Combined organic extracts were washed with brine, dried over anhydrous sodium sulfate and evaporated under reduced pressure to afford the title compound 4.

### Step 3: Synthesis of 4-(1-methyl-1H-pyrrol-2-yl) aniline (5)

The title compound has been synthesized by following the general procedure described above for reduction using the nitro compound 4 and Fe/NH₄Cl.

### Step 4

6-(1-methyl-1H-indazol-6-yl)-N-(4-(1-methyl-1H-pyrrol-2-yl)phenyl)-2-morpholino pyrimidin-4-amine: The title compound has been synthesized by following the general procedure described above for displacement reaction (IPA, Conc. HCl) by using corresponding chloro compound 6 and amine 5. ¹H NMR (400 MHz, DMSO-d₆) δ: 10.05 (s, 1H), 8.23 (s, 1H), 8.14 (s, 1H), 7.90 (d, J=8.4 Hz, 1H), 7.72 (dd, J=22.7, 8.3 Hz, 3H), 7.43 (d, J=8.1 Hz, 2H), 6.82 (s, 1H), 6.74 (s, 1H), 6.17-6.11 (m, 1H), 6.05 (t, J=3.2 Hz, 1H), 4.14 (s, 3H), 3.84-3.75 (m, 8H), 3.66 (s, 3H); HPLC purity: 95.23%; LCMS Calculated for C₂₇H₂₇N₇O (free base): 465.23; Observed: 466.25 (M+1).

6-(1,3-dimethyl-1H-indazol-6-yl)-N-(4-(1-methyl-1H-pyrrol-2-yl)phenyl)-2-morpholinopyrimidin-4-amine: The title compound has been synthesized by following the general procedure described above for displacement reaction (IPA, Conc. HCl) by using corresponding chloro compound 6 and amine 5. ¹H NMR (400 MHz, DMSO-d₆) δ: 9.95 (s, 1H), 8.15 (s, 1H), 7.85 (d, J=8.5 Hz, 1H), 7.75 (d, J=8.2 Hz, 2H), 7.63 (d, J=8.1 Hz, 1H), 7.44 (d, J=8.2 Hz, 2H), 6.82 (t, J=2.3 Hz, 1H), 6.74 (s, 1H), 6.14 (t, J=2.7 Hz, 1H), 6.05 (t, J=3.1 Hz, 1H), 4.05 (s, 3H), 3.84-3.75 (m, 8H), 3.66 (s, 3H), 2.49 (s, 3H); HPLC purity: 96.75%; LCMS Calculated for C₂₈H₂₉N₇O (free base): 479.24; Observed: 480.50 (M+1).

### Example 121

### Synthesis of N-(4-(1H-pyrrol-3-yl)phenyl)-6-(1,3-dimethyl-1H-indazol-6-yl)-2-morpholinopyrimidin-4-amine

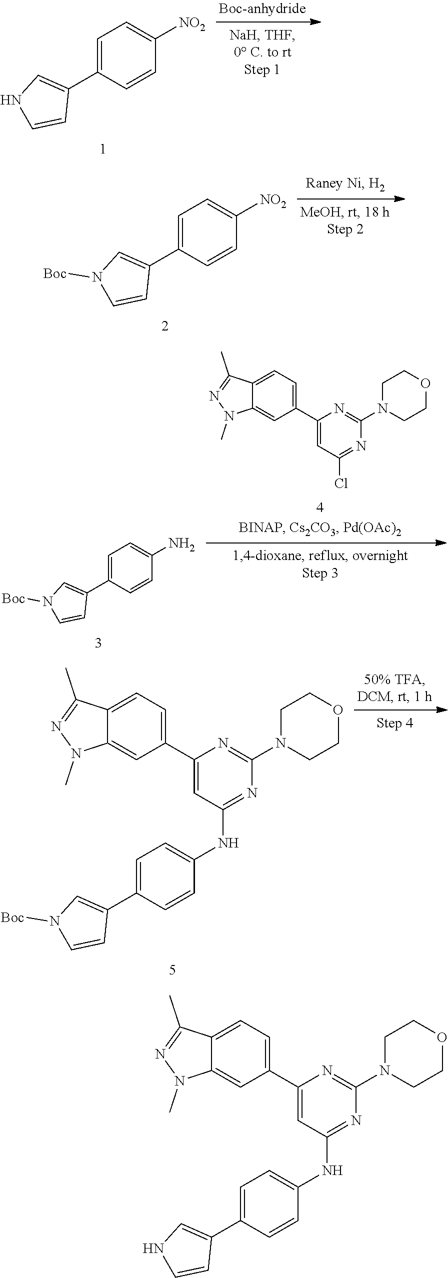

### Step 1: Synthesis of tert-butyl 3-(4-nitrophenyl)-1H-pyrrole-1-carboxylate (2)

To a stirred solution of compound 1 (1.3 g, 1 eq) in THF (40 mL), NaH (0.248 g, 1.5 eq) was added at 0° C. and stirred for 15 min followed by the addition of Boc anhydride (1.80 g, 1.2 eq). The reaction mixture was stirred at room temperature for 2 h. The progress of the reaction was monitored by TLC. After completion of the reaction, the reaction mixture was diluted with water and extracted with ethyl acetate (2×50 mL). Combined organic extracts were washed with brine, dried over anhydrous sodium sulfate and evaporated under reduced pressure to afford the title compound 2.

### Step 2: Synthesis of tert-butyl 3-(4-aminophenyl)-1H-pyrrole-1-carboxylate (3

To a stirred solution of compound 2 (0.6 g, 1 eq) in methanol (30 mL), Raney nickel (0.12 g) was added under nitrogen atmosphere. The reaction mixture was stirred at room temperature for 18 h under hydrogen atmosphere (balloon pressure). The progress of the reaction was monitored by TLC. After completion of the reaction, the reaction mixture was filtered through celite and evaporated under reduced pressure to afford the title compound 3. LCMS (m/z): 259.05 (M+1).

Step 3: Synthesis of tert-butyl 3-(4-((6-(1,3-dimethyl-1H-indazol-6-yl)-2-morpholinopyrimidin-4-yl) amino)phenyl)-1H-pyrrole-1-carboxylate (5)

The title compound has been synthesized by following the General procedure for Buchwald Coupling described above using chloro compound 4 and amine 3. LCMS (m/z): 566.55 (M+1).

### Step 4: Synthesis of N-(4-(1H-pyrrol-3-yl)phenyl)-6-(1,3-dimethyl-1H-indazol-6-yl)-2-morpholino pyrimidin-4-amine

To a stirred solution of compound 5 (0.15 g, 1 eq) in dichloromethane (2 mL), TFA (2 mL) was added and stirred at room temperature for 1 h. The progress of the reaction was monitored by TLC. After completion of the reaction, the reaction mixture was evaporated under reduced pressure. The residue was neutralized with saturated sodium bicarbonate solution and extracted with ethyl acetate (2×25 mL). Combined organic extracts were dried over anhydrous sodium sulfate and evaporated under reduced pressure. The crude product was purified by column chromatography on silica gel 100-200 mesh using 70% EtOAc-hexane to afford the title compound. ¹H NMR (400 MHz, DMSO-d₆) δ: 10.84 (s, 1H), 9.33 (s, 1H), 8.15 (s, 1H), 7.82-7.67 (m, 2H), 7.62 (d, J=8.2 Hz, 2H), 7.49 (d, J=8.2 Hz, 2H), 7.19-7.13 (m, 1H), 6.77 (d, J=2.8 Hz, 1H), 6.69 (s, 1H), 6.41 (s, 1H), 4.03 (s, 3H), 3.82-3.74 (m, 8H), 2.42 (s, 3H); HPLC purity: 96.84%; LCMS Calculated for C₂₇H₂₇N₇O: 465.23; Observed: 466.30 (M+1).

### Examples 122-123

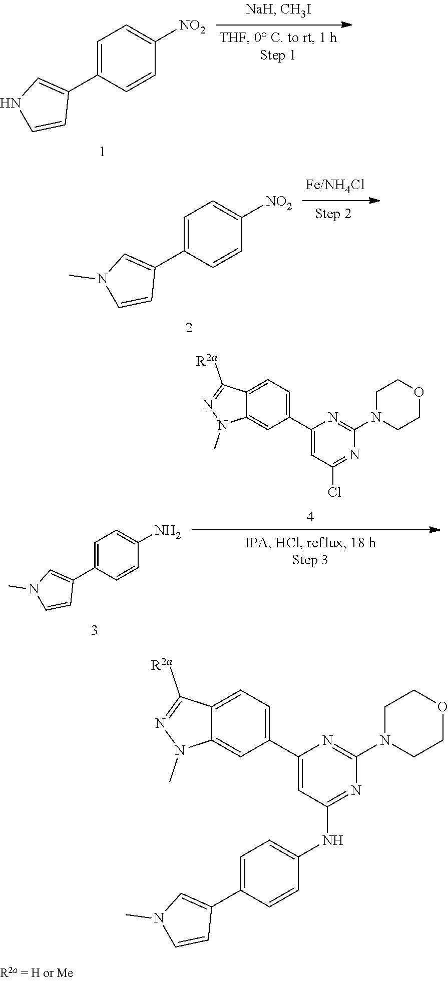

### Step 1: Synthesis of 1-methyl-3-(4-nitrophenyl)-1H-pyrrole (2)

To a stirred solution of 1 (0.6 g, 1 eq) in DMF (8 mL), NaH (0.191 g, 1.5 eq) was added at 0° C. and stirred for 15 min followed by the addition of methyl iodide (0.29 mL, 1.5 eq). The reaction mixture was stirred at room temperature for 1 h. The progress of the reaction was monitored by TLC. After completion of the reaction, the reaction mixture was diluted with water and extracted with ethyl acetate (2×25 mL). Combined organic extracts were washed with brine, dried over anhydrous sodium sulfate and evaporated under reduced pressure to afford the title compound 2.

### Step 2: Synthesis of 4-(1-methyl-1H-pyrrol-3-yl) aniline (3)

The title compound has been synthesized by following the general procedure described above for reduction using the nitro compound 2 and Fe/NH₄Cl. LCMS (m/z): 173.00 (M+1).

### Step 3

6-(1-methyl-1H-indazol-6-yl)-N-(4-(1-methyl-1H-pyrrol-3-yl)phenyl)-2-morpholino pyrimidin-4-amine: The title compound has been synthesized by following the general procedure described above for displacement reaction (IPA, Conc. HCl) by using corresponding chloro compound 4 and amine 3. ¹H NMR (400 MHz, DMSO-d₆) δ: 9.36 (s, 1H), 8.23 (s, 1H), 8.09 (s, 1H), 7.84 (d, J=8.5 Hz, 1H), 7.76 (dd, J=8.6, 1.3 Hz, 1H), 7.67-7.58 (m, 2H), 7.49-7.42 (m, 2H), 7.13-7.07 (m, 1H), 6.77-6.67 (m, 2H), 6.36 (s, 1H), 4.13 (s, 3H), 3.82-3.69 (m, 8H), 3.63 (s, 3H); HPLC purity: 99.22%; LCMS Calculated for C₂₇H₂₇N₇O: 465.23; Observed: 466.30 (M+1).

6-(1,3-dimethyl-1H-indazol-6-yl)-N-(4-(1-methyl-1H-pyrrol-3-yl)phenyl)-2-morpholinopyrimidin-4-amine: The title compound has been synthesized by following the general procedure described above for displacement reaction (IPA, Conc. HCl) by using corresponding chloro compound 4 and amine 3. ¹H NMR (400 MHz, DMSO-d₆) δ: 10.19 (s, 1H), 8.13 (s, 1H), 7.87 (d, J=8.3 Hz, 1H), 7.62 (d, J=8.1 Hz, 3H), 7.52 (t, J=12.0 Hz, 2H), 7.15 (s, 1H), 6.72 (d, J=12.0 Hz, 2H), 6.39 (s, 1H), 4.05 (s, 3H), 3.85-3.75 (m, 8H), 3.64 (s, 3H), 2.60 (s, 3H); HPLC purity: 96.31%; LCMS Calculated for C₂₈H₂₉N₇O: 479.24; Observed: 480.40 (M+1).

### Example 124

### Synthesis of N-(4-(5-methyl-4H-1,2,4-triazol-3-yl)phenyl)-2-morpholino-6-(1-(2-morpholinoethyl)-1H-indazol-6-yl)pyrimidin-4-amine

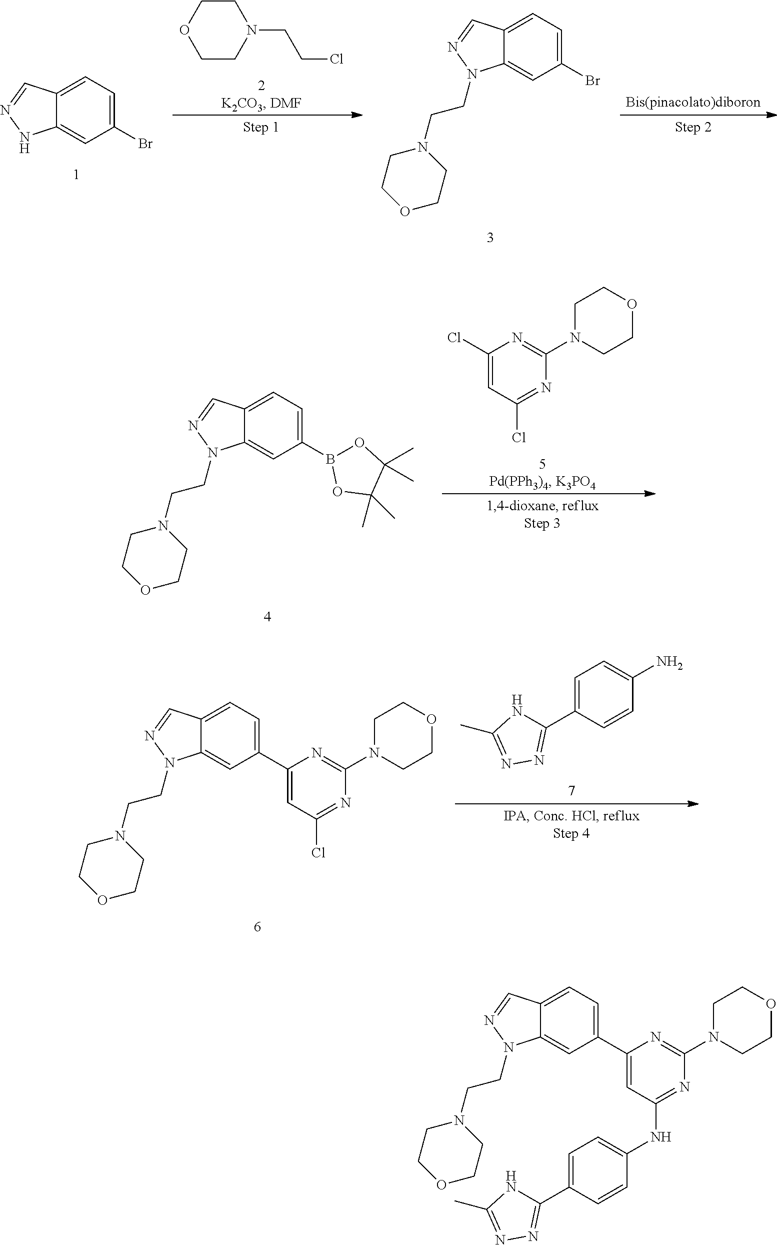

### Step 1: Synthesis of 4-(2-(6-bromo-1H-indazol-1-yl) ethyl)morpholine (3)

To a stirred solution of compound 1 (1.5 g, 1 eq) in DMF (20 mL), compound 2 (1.84 g, 1.3 eq) and K₂CO₃ (3.1 g, 3 eq) were added at room temperature and stirred at 80° C. for 24 h. The progress of the reaction was monitored by TLC. After completion of the reaction, the reaction mixture was quenched with water and extracted with ethyl acetate (2×50 mL). Combined organic extracts were washed with brine, dried over anhydrous sodium sulfate and evaporated under reduced pressure. The crude product was purified by column chromatography on silica gel 100-200 mesh using 80% EtOAc-hexane to afford the title compound 3. LCMS (m/z): 309.95 (M+1).

### Step 2: Synthesis of 4-(2-(6-(4,4,5,5-tetramethyl-1,3,2-dioxaborolan-2-yl)-1H-indazol-1-yl)ethyl) morpholine (4)

The title compound has been synthesized by following the General Procedure for Boronate Ester Preparation described above using bromo compound 3 and Bis (pinacolato)diboron. LCMS (m/z): 358.35 (M+1).

### Step 3: Synthesis of 4-(2-(6-(6-chloro-2-morpholinopyrimidin-4-yl)-1H-indazol-1-yl)ethyl)morpholine (6)

The title compound has been synthesized by following the General Procedure for Suzuki Coupling described above using compound 5 and Boronate ester 4. LCMS (m/z): 429.35 (M+1).

Step 4: Synthesis of N-(4-(5-methyl-4H-1,2,4-triazol-3-yl)phenyl)-2-morpholino-6-(1-(2-morpholino ethyl)-1H-indazol-6-yl)pyrimidin-4-amine

The title compound has been synthesized by following the general procedure described above for displacement reaction (IPA, Conc. HCl) by using chloro compound 6 and amine 7. ¹H NMR (400 MHz, DMSO-d₆) δ: 13.53 (s, 1H), 9.56 (s, 1H), 8.29 (s, 1H), 8.11 (s, 1H), 7.93 (d, J=8.3 Hz, 2H), 7.84 (d, J=8.4 Hz, 2H), 7.76 (d, J=8.5 Hz, 2H), 6.74 (s, 1H), 4.61 (t, J=6.4 Hz, 2H), 3.82-3.73 (m, 8H), 3.48 (t, J=4.5 Hz, 4H), 2.79 (t, J=6.5 Hz, 2H), 2.50 (s, 3H), 2.44-2.39 (m, 4H); HPLC purity: 99.93%; LCMS Calculated for C₃₀H₃₄N₁₀O₂: 566.29; Observed: 567.55 (M+1).

### Example 125

### Synthesis of 6-(3-methyl-1-(2-morpholinoethyl)-1H-indazol-6-yl)-N-(4-(5-methyl-4H-1,2,4-triazol-3-yl) phenyl)-2-morpholinopyrimidin-4-amine

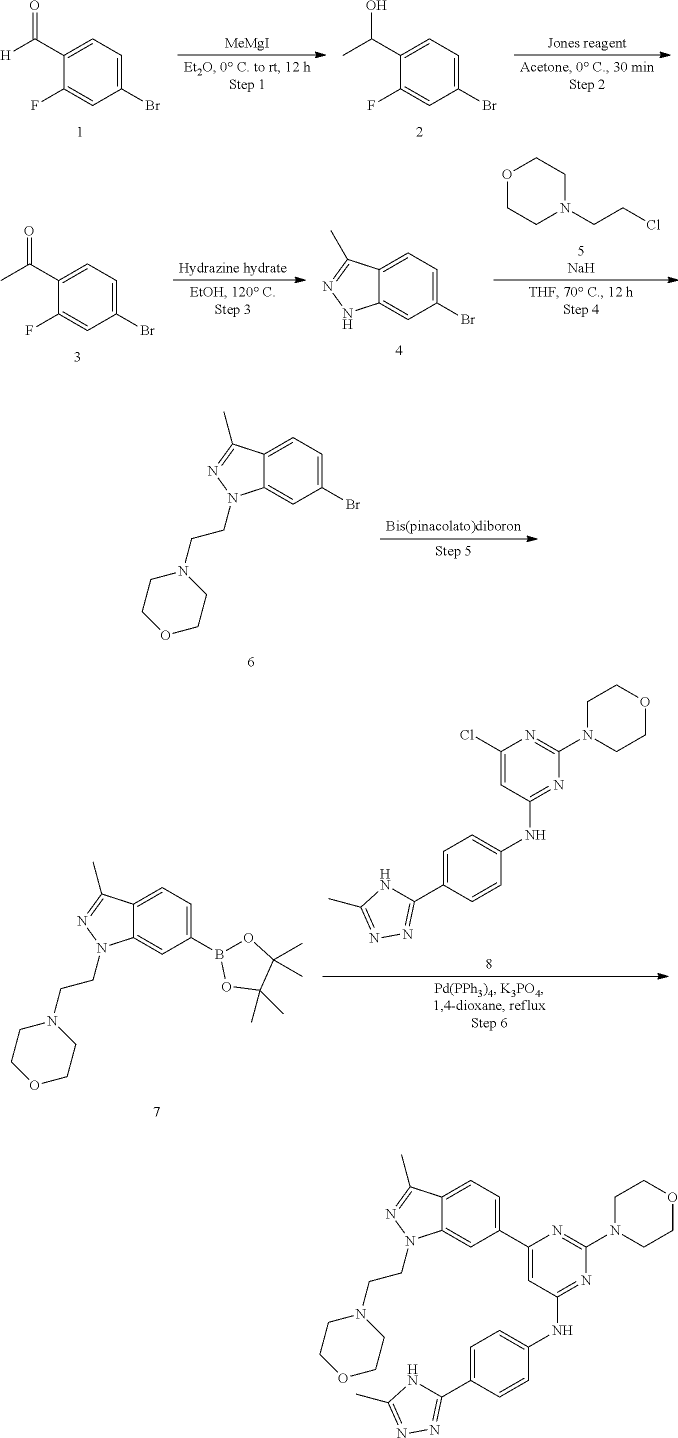

### Step 1: Synthesis of 1-(4-bromo-2-fluorophenyl)ethanol (2)

To a stirred solution of compound 1 (10 g, 1 eq) in diethyl ether (100 mL), methyl magnesium iodide (17.6 g, 3 eq) was added at 0° C. and stirred at same temperature for 3 h. The progress of the reaction was monitored by TLC. After completion of the reaction, the reaction mixture was quenched with saturated ammonium chloride solution and extracted with ethyl acetate (3×100 mL). Combined organic extracts were washed with brine, dried over anhydrous sodium sulfate and evaporated under reduced pressure to afford the title compound 2. ¹H NMR (400 MHz, DMSO-d₆) δ: 7.49-7.39 (m, 3H), 5.38 (s, 1H), 4.95-4.89 (m, 1H), 1.31 (d, J=6.4 Hz, 3H).

### Step 2: Synthesis of 1-(4-bromo-2-fluorophenyl)ethanone (3)

To a stirred solution of compound 2 (10 g, 1 eq) in acetone (100 mL), Jones reagent (12 mL) was added at 0° C. and stirred at same temperature for 30 min. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure to afford the title compound 3. ¹H NMR (400 MHz, CDCl₃) δ: 7.76-7.72 (m, 1H), 7.37-7.31 (m, 2H), 2.61 (s, 3H).

### Step 3: Synthesis of 6-bromo-3-methyl-1H-indazole (4)

A stirred solution of compound 3 (9 g, 1 eq) in hydrazine hydrate (9 mL) was refluxed for 18 h. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure to afford the title compound 4. LCMS (m/z): 210.95 (M+1).

### Step 4: Synthesis of 4-(2-(6-bromo-3-methyl-1H-indazol-1-yl)ethyl)morpholine (6)

To a stirred suspension of NaH (1.14 g, 3 eq) in THF (50 mL), compound 4 (2 g, 1 eq) (solution in THF) was added at 0° C. and stirred at room temperature for 1 h. Compound 5 (2.65 g, 1.5 eq) was added and the reaction mixture was heated at 70° C. for 18 h. The progress of the reaction was monitored by TLC. After completion of the reaction, the reaction mixture was quenched with water and extracted with ethyl acetate (3×25 mL). Combined organic extracts were washed with brine, dried over anhydrous sodium sulfate and evaporated under reduced pressure. The crude product was purified by column chromatography on silica gel 100-200 mesh using 50% EtOAc-hexane to afford the title compound 6. LCMS (m/z): 326.15 (M+2).

### Step 5: Synthesis of 4-(2-(3-methyl-6-(4,4,5,5-tetramethyl-1,3,2-dioxaborolan-2-yl)-1H-indazol-1-yl) ethyl)morpholine (7)

The title compound (crude product) has been synthesized by following the General Procedure for Boronate Ester Preparation described above using bromo compound 6 and Bis (pinacolato)diboron. LCMS (m/z): 372.35 (M+1).

### Step 6: Synthesis of 6-(3-methyl-1-(2-morpholinoethyl)-1H-indazol-6-yl)-N-(4-(5-methyl-4H-1,2,4-triazol-3-yl) phenyl)-2-morpholinopyrimidin-4-amine

The title compound has been synthesized by following the General Procedure for Suzuki Coupling described above using chloro compound 8 and Boronate ester 7. ¹H NMR (400 MHz, MeOD) δ: 8.36 (s, 1H), 8.19 (s, 1H), 7.91 (d, J=8.3 Hz, 2H), 7.83-7.70 (m, 4H), 6.66 (s, 1H), 4.55 (t, J=6.6 Hz, 2H), 3.91-3.81 (m, 8H), 3.61 (t, J=4.6 Hz, 4H), 2.87 (t, J=6.6 Hz, 2H), 2.59 (s, 3H), 2.55-2.44 (m, 4H), 2.42 (s, 3H); HPLC purity: 97.76%; LCMS Calculated for C₃₁H₃₆N₁₀O₂: 580.30; Observed: 581.45 (M+1).

### Example 126

### Synthesis of 6-(1-methyl-3-(2-morpholinoethyl)-1H-indazol-6-yl)-N-(4-(5-methyl-4H-1,2,4-triazol-3-yl) phenyl)-2-morpholinopyrimidin-4-amine

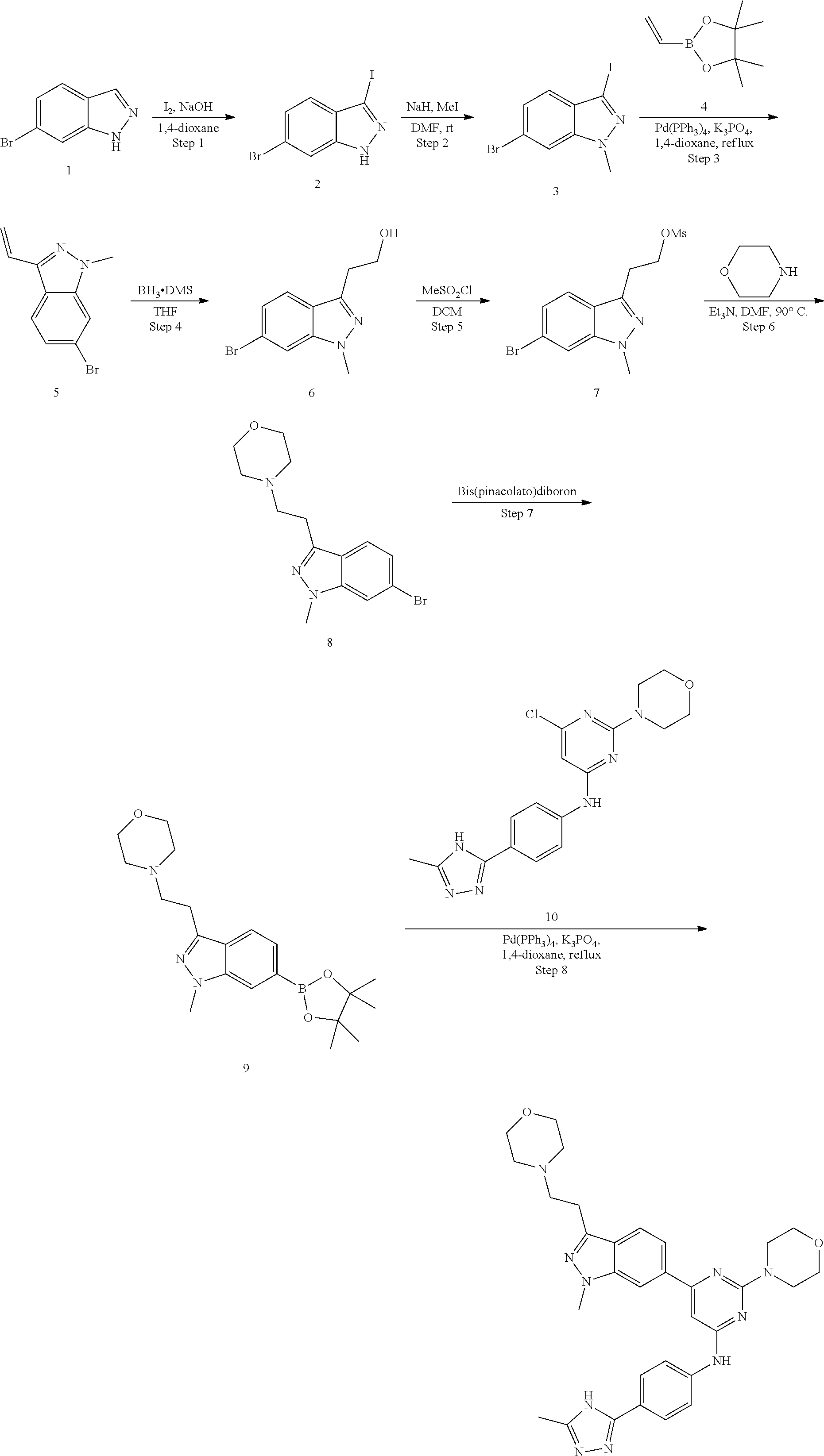

### Step 1: Synthesis of 6-bromo-3-iodo-1H-indazole (2)

To a stirred solution of compound 1 (10 g, 1 eq) in 1,4-dioxane (40 mL), 3N NaOH (100 mL) solution, Iodine (28.51 g, 2.2 eq) was added and stirred at room temperature for 1 h. The progress of the reaction was monitored by TLC. After completion of the reaction, the reaction mixture was quenched with 20% citric acid solution, saturated sodium bicarbonate solution and extracted with ethyl acetate (3×50 mL). Combined organic extracts were washed with brine, dried over anhydrous sodium sulfate and evaporated under reduced pressure to afford the title compound 2. LCMS (m/z): 323.00 (M+1).

### Step 2: Synthesis of 6-bromo-3-iodo-1-methyl-1H-indazole (3)

To a stirred solution of compound 2 (16.3 g, 1 eq) in DMF (150 mL), NaH (1.82 g, 1.5 eq) was added and stirred at room temperature for 10 min followed by the addition of methyl iodide (14.37 g, 2 eq). The reaction mixture was stirred for 30 min at room temperature. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 10% EtOAc-hexane to afford the title compound 3. LCMS (m/z): 338.85 (M+1).

### Step 3: Synthesis of 6-bromo-1-methyl-3-vinyl-1H-indazole (5)

The title compound has been synthesized by following the General Procedure for Suzuki Coupling described above using compound 3 and Boronate ester 4. LCMS (m/z): 236.95 (M+1).

### Step 4: Synthesis of 2-(6-bromo-1-methyl-1H-indazol-3-yl) ethanol (6)

To a stirred solution of compound 5 (2.1 g, 1 eq) in dry THF (50 mL), BH3:DMS (3.2 mL, 4 eq) was added at 0° C. and stirred at room temperature for 16 h. The progress of the reaction was monitored by TLC. After completion of the reaction, the reaction mixture was quenched with 3N NaOH and 30% H₂O₂ solution at 0° C. The reaction mixture was stirred at room temperature for 3 h and extracted with ethyl acetate (3×50 mL). Combined organic extracts were dried over anhydrous sodium sulfate and evaporated under reduced pressure. The crude product was purified by column chromatography on silica gel 100-200 mesh using 60% EtOAc-hexane to afford the title compound 6. LCMS (m/z): 257.00 (M+2).

### Step 5: Synthesis of 2-(6-bromo-1-methyl-1H-indazol-3-yl) ethyl methanesulfonate (7)

To a stirred solution of compound 6 (1.9 g, 1 eq) in dichloromethane (10 mL), TEA (0.206 g, 2 eq) was added at room temperature and stirred for 15 min. Mesyl chloride (0.175 g, 1.5 eq) was added to the reaction mixture at 0° C. and stirred at room temperature for 1 h. The progress of the reaction was monitored by TLC. After completion of the reaction, the reaction mixture was quenched with saturated sodium bicarbonate solution and extracted with ethyl acetate (3×50 mL). Combined organic extracts were dried over anhydrous sodium sulfate and evaporated under reduced pressure to afford the title compound 7. LCMS (m/z): 334.05 (M+1).

### Step 6 : Synthesis of 4-(2-(6-bromo-1-methyl-1H-indazol-3-yl)ethyl)morpholine (8)

To a stirred solution of morpholine (0.083 g, 1 eq) in DMF (5 mL), TEA (0.194 g, 2 eq) was added at room temperature and stirred for 15 min followed by the addition of compound 7 (0.31 g, 1 eq; as a solution in DMF). The reaction mixture was heated in a sealed tube at 90° C. for 1 h. The progress of the reaction was monitored by TLC. After completion of the reaction, the reaction mixture was diluted with water and extracted with ethyl acetate (3×20 mL). Combined organic extracts were dried over anhydrous sodium sulfate and evaporated under reduced pressure. The crude product was purified by column chromatography on silica gel 100-200 mesh using 5% MeOH-DCM to afford the title compound 8. LCMS (m/z): 324.10 (M+1).

### Step 7: Synthesis of 4-(2-(1-methyl-6-(4,4,5,5-tetramethyl-1,3,2-dioxaborolan-2-yl)-1H-indazol-3-yl) ethyl)morpholine (9)

The title compound (crude product) has been synthesized by following the General Procedure for Boronate Ester Preparation described above using bromo compound 8 and Bis(pinacolato)diboron. LCMS (m/z): 372.40 (M+1).

### Step 8: Synthesis of 6-(1-methyl-3-(2-morpholinoethyl)-1H-indazol-6-yl)-N-(4-(5-methyl-4H-1,2,4-triazol-3-yl) phenyl)-2-morpholinopyrimidin-4-amine

The title compound has been synthesized by following the General Procedure for Suzuki Coupling described above using chloro compound 10 and Boronate ester 9. ¹H NMR (400 MHz, DMSO-d₆) δ: 9.61 (s, 1H), 8.17 (s, 1H), 7.93 (d, J=8.3 Hz, 2H), 7.78 (td, J=24.7, 22.5, 8.4 Hz, 4H), 6.74 (s, 1H), 4.13 (s, 1H), 4.05 (s, 3H), 3.86-3.74 (m, 8H), 3.59-3.55 (m, 4H), 3.36-3.30 (m, 4H), 3.09 (t, J=7.8 Hz, 2H), 2.70 (t, J=7.9 Hz, 2H), 2.36 (s, 3H); HPLC purity: 98.25%; LCMS Calculated for C₃₁H₃₆N₁₀O₂: 580.30; Observed: 581.60 (M+1).

### Example 127

### Synthesis of N-(4-chlorophenyl)-5-methyl-6-(1-methyl-1H-indazol-6-yl)-2-morpholinopyrimidin-4-amine

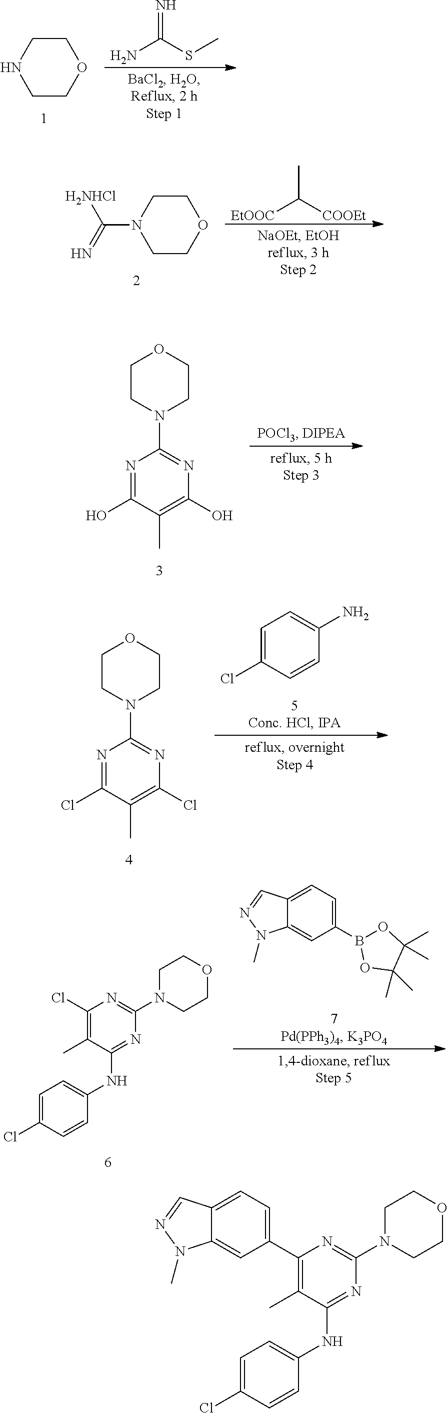

### Step 1: Synthesis of morpholine-4-carboximidamide (2)

To a stirred solution of compound 1 (10 g, 1 eq) in water (26 mL), barium chloride solution (11.97 g, 0.8 eq) in water (18 mL) was added at 100° C. and refluxed for 2 h. After completion of the reaction, the reaction mixture was filtered. The filtrate was evaporated to dryness. The residue was dissolved in solution of ethanol: acetone (1:5; 450 mL), stirred for 30 min and filtered. The solid obtained was dried under vacuum to afford the title compound 2. LCMS (m/z): 129.95 (M+1).

### Step 2: Synthesis of 5-methyl-2-morpholinopyrimidine-4, 6-diol (3)

To a stirred solution of ethanol (200 mL), sodium metal (4.92 g, 3 eq) was added slowly under nitrogen atmosphere. After dissolution of sodium metal, compound 2 (9.2 g, 1 eq) and diethyl 2-methylmalonate (12.4 g, 1 eq) were added. The reaction mixture was refluxed for 3 h. The progress of the reaction was monitored by TLC. After completion of the reaction, the reaction mixture was evaporated to dryness. The residue was diluted with water, acidified to pH 2 using 1N HCl and filtered. The solid obtained was dried under vacuum to afford the title compound 3. LCMS (m/z): 212.00 (M+1).

### Step 3: Synthesis of 4-(4,6-dichloro-5-methylpyrimidin-2-yl)morpholine (4)

To a stirred solution of compound 3 (7.4 g, 1 eq) in phosphorous oxychloride (60 mL), DIPEA (0.29 g, 2 eq) was added slowly and was heated to reflux for 5 h. The progress of the reaction was monitored by TLC. After completion of the reaction, excess phosphorous oxychloride was distilled off. The residue was quenched with ice and extracted with ethyl acetate (3×100 mL). Combined organic extracts were washed with brine, dried over anhydrous sodium sulfate and evaporated under reduced pressure. The crude product was purified by column chromatography on silica gel 100-200 mesh using 10% EtOAc-hexane to afford the title compound 4. LCMS (m/z): 247.95 (M+1).

### Step 4: Synthesis of 6-chloro-N-(4-chlorophenyl)-5-methyl-2-morpholinopyrimidin-4-amine (6)

The title compound has been synthesized by following the general procedure described above for displacement reaction (IPA, Conc. HCl) by using chloro compound 4 and p-chloro aniline 5. LCMS (m/z): 339.10 (M+1).

### Step 5: Synthesis of N-(4-chlorophenyl)-5-methyl-6-(1-methyl-1H-indazol-6-yl)-2-morpholinopyrimidin-4-amine

The title compound has been synthesized by following the General Procedure for Suzuki Coupling described above using chloro compound 6 and Boronate ester 7. ¹H NMR (400 MHz, DMSO-d₆) δ: 8.46 (s, 1H), 8.09 (s, 1H), 7.81 (d, J=8.3 Hz, 1H), 7.73 (d, J=9.1 Hz, 3H), 7.41-7.34 (m, 2H), 7.24 (dd, J=8.3, 1.3 Hz, 1H), 4.08 (s, 3H), 3.66-3.58 (m, 8H), 2.11 (s, 3H); HPLC purity: 95.05%; LCMS Calculated for C₂₃H₂₃ClN₆O: 434.16; Observed: 435.30 (M+1).

### Example 128

### Synthesis of N-(4-chlorophenyl)-6-(1-(2-(4-methylpiperazin-1-yl)ethyl)-1H-indazol-6-yl)-2-morpholino pyrimidin-4-amine

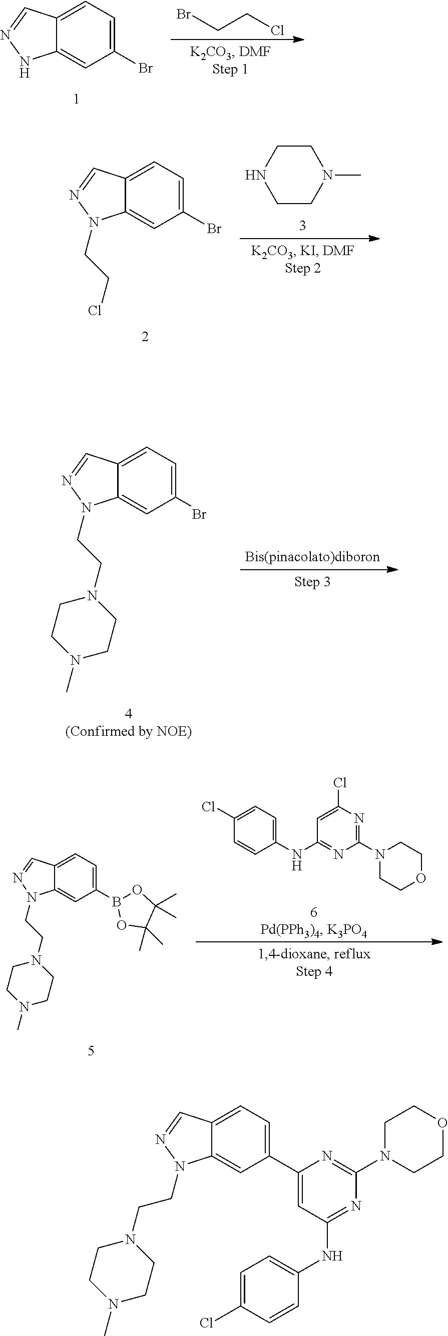

### Step 1: Synthesis of 6-bromo-1-(2-chloroethyl)-1H-indazole (2)

To a stirred solution of compound 1 (1.5 g, 1 eq) in acetonitrile (15 mL), K₂CO₃ (3.15 g, 3 eq) was added followed by the addition of 1-bromo-2-chloroethane (0.78 mL, 1.2 eq). The reaction mixture was stirred at 90° C. for 18 h. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 30% EtOAc-hexane to afford the title compound 2.
LCMS (m/z): 261.00 (M+2).

### Step 2: Synthesis of 6-bromo-1-(2-(4-methylpiperazin-l-yl)ethyl)-1H-indazole (4)

To a stirred solution of compound 2 (1.1 g, 1 eq) and compound 3 (0.57 mL, 1g) in DMF (15 mL), K₂CO₃ (1.47 g, 2.5 eq) was added followed by the addition of KI (0.212 g, 0.3 eq). The reaction mixture was stirred at 80° C. for 4 h. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 5% MeOH-DCM to afford the title compound 4. LCMS (m/z): 323.10 (M+1).

### Step 3: Synthesis of 1-(2-(4-methylpiperazin-1-yl)ethyl)-6-(4,4,5,5-tetramethyl-1,3,2-dioxaborolan-2-yl) -1H-indazole (5)

The title compound have been synthesized by following the General Procedure for Boronate Ester Preparation described above using bromo compound 4 and Bis (pinacolato)diboron. LCMS (m/z): 371.35 (M+1).

### Step 4: Synthesis of N-(4-chlorophenyl)-6-(1-(2-(4-methylpiperazin-1-yl)ethyl)-1H-indazol-6-yl)-2-morpholino pyrimidin-4-amine

The title compound has been synthesized by following the General Procedure for Suzuki Coupling described above using compound 6 and Boronate ester 5. ¹H NMR (400 MHz, DMSO-d₆) δ: 11.66 (s, 1H), 10.25 (s, 1H), 8.44 (s, 1H), 8.25 (s, 1H), 7.93 (d, J=8.5 Hz, 1H), 7.79-7.66 (m, 3H), 7.42 (d, J=8.7 Hz, 2H), 6.80 (s, 1H), 5.00 (d, J=7.1 Hz, 2H), 3.85 (d, J=4.8 Hz, 4H), 3.78-3.62 (m, 10H), 3.44-3.29 (m, 4H), 2.82 (s, 3H); HPLC purity: 99.87%; LCMS Calculated for C₂₈H₃₃ClN₈₀ (free base): 532.25; Observed: 533.40 (M+1).

### Example 129

### Synthesis of N-(4-chlorophenyl)-6-(1-(3-(4-methylpiperazin-1-yl)propyl)-1H-indazol-6-yl)-2-morpholino pyrimidin-4-amine

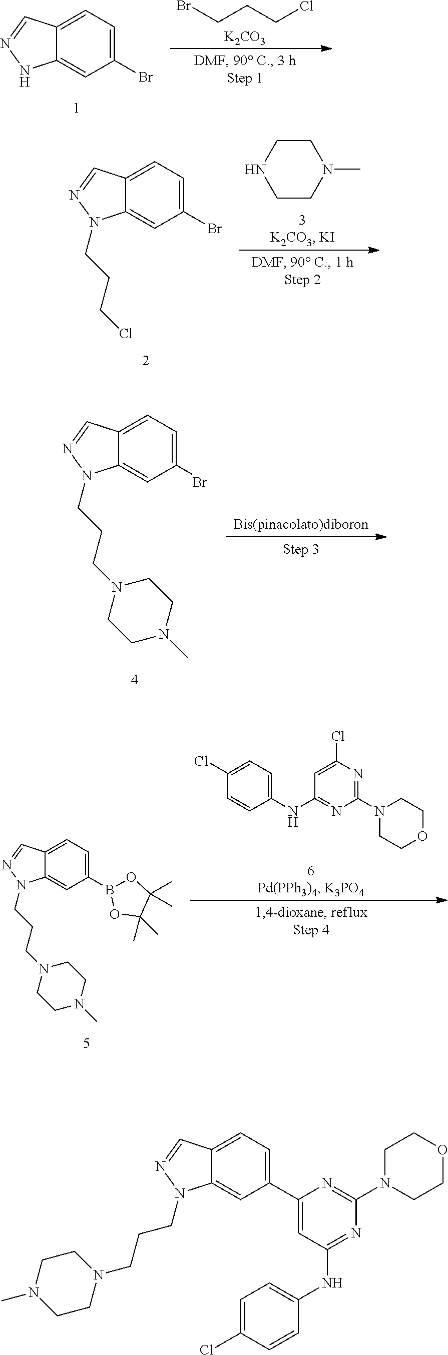

### Step 1: Synthesis of 6-bromo-1-(3-chloropropyl)-1H-indazole (2)

To a stirred solution of compound 1 (2 g, 1 eq) in DMF (30 mL), K₂CO₃ (4.2 g, 3 eq) was added followed by the addition of 1-bromo-3-chloropropane (3.18 g, 2 eq). The reaction mixture was stirred at 90° C. for 3 h. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 30% EtOAc-hexane to afford the title compound 2. LCMS (m/z): 274.95 (M+2).

### Step 2: Synthesis of 6-bromo-1-(3-(4-methylpiperazin-l-yl)propyl)-1H-indazole (4)

To a stirred solution of compound 2 (0.78 g, 1 eq) and compound 3 (0.34 g, 1 g) in DMF (10 mL), K₂CO₃ (0.98 g, 2.5 eq) was added followed by the addition of KI (0.141 g, 0.3 eq). The reaction mixture was stirred at 90° C. for 1 h. The progress of the reaction was monitored by TLC. After completion of the reaction, the reaction mixture was diluted with water and extracted with ethyl acetate (3×25 mL). Combined organic extracts were washed with brine, dried over anhydrous sodium sulfate and evaporated under reduced pressure. The crude product was purified by column chromatography on silica gel 100-200 mesh using 100% ethyl acetate to afford the title compound 4. LCMS (m/z): 337.15 (M+1).

### Step 3: Synthesis of 1-(3-(4-methylpiperazin-1-yl)propyl)-6-(4,4,5,5-tetramethyl-1,3,2-dioxaborolan-2-yl)-1H-indazole (5)

The title compound have been synthesized by following the General Procedure for Boronate Ester Preparation described above using bromo compound 4 and Bis (pinacolato)diboron. LCMS (m/z): 385.35 (M+1).

### Step 4: Synthesis of N-(4-chlorophenyl)-6-(1-(3-(4-methylpiperazin-1-yl)propyl)-1H-indazol-6-yl)-2-morpholino pyrimidin-4-amine

The title compound has been synthesized by following the General Procedure for Suzuki Coupling described above using chloro compound 6 and Boronate ester 5. ¹H NMR (400 MHz, DMSO-d₆) δ: 9.58 (s, 1H), 8.26 (s, 1H), 8.12 (s, 1H), 7.84 (d, J=8.4 Hz, 1H), 7.79-7.68 (m, 3H), 7.41-7.33 (m, 2H), 6.70 (s, 1H), 4.51 (t, J=6.5 Hz, 2H), 3.84-3.69 (m, 8H), 2.56-2.40 (m, 4H), 2.36-2.18 (m, 6H), 2.02-1.98 (m, 2H); HPLC purity: 99.51%; LCMS Calculated for C₂₉H₃₅ClN₈O (free base): 546.26; Observed: 547.40 (M+1).

### Example 130-132

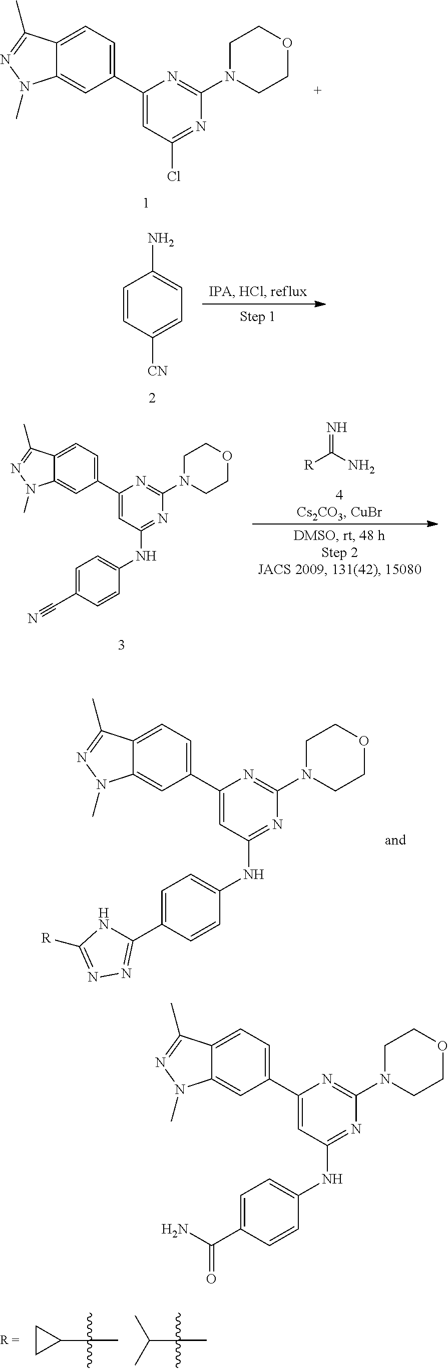

Step 1: Synthesis of 4-((6-(1,3-dimethyl-1H-indazol-6-yl)-2-morpholinopyrimidin-4-yl)amino) benzonitrile (3)

The title compound has been synthesized by following the general procedure described above for displacement reaction (IPA/HCl) by using chloro compound 1 and amino compound 2. LCMS (m/z): 426.20 (M+1).

### Step 2

N-(4-(5-cyclopropyl-4H-1,2,4-triazol-3-yl)phenyl)-6-(1,3-dimethyl-1H-indazol-6-yl)-2-morpholinopyrimidin-4-amine: To a stirred solution of compound 3 (0.2 g, 1 eq) in DMSO (5 mL), copper bromide (0.005 g, 0.05 eq), Cs₂CO₃ (0.458 g, 3 eq) was added and stirred for 10 min followed by the addition of cyclopropane carboximidamide hydrochloride (0.085 g,1.5 eq) at room temperature. The reaction mixture was stirred at room temperature for 48 h. The progress of the reaction was monitored by TLC. After completion of the reaction, the reaction mixture was quenched with saturated sodium bicarbonate solution and extracted with ethyl acetate (3×20 mL). Combined organic extracts were washed with brine, dried over anhydrous sodium sulfate and evaporated under reduced pressure. The crude product was purified by preparative HPLC to afford the title compound. HPLC purity: 99.25%; ¹H NMR (400 MHz, DMSO-d₆) δ: 10.29 (s, 1H), 8.16 (s, 1H), 8.00 (d, J=8.4 Hz, 2H), 7.89-7.82 (m, 3H), 7.63 (d, J=8.4 Hz, 1H), 6.79 (s, 1H), 4.05 (s, 3H), 3.85-3.75 (m, 8H), 2.36 (s, 3H), 2.14 (td, J=8.4, 4.2 Hz, 1H), 1.15-1.07 (m, 4H); LCMS Calculated for C₂₈H₂₉N₉₀ (free base): 507.25; Observed: 508.30 (M+1).

6-(1,3-dimethyl-1H-indazol-6-yl)-N-(4-(5-isopropyl-4H-1,2,4-triazol-3-yl)phenyl)-2-morpholinopyrimidin-4-amine: The title compound has been synthesized by following the procedure described above for N-(4-(5-cyclopropyl-4H-1,2,4-triazol-3-yl)phenyl)-6-(1,3-dimethyl-1H-indazol-6-yl)-2-morpholinopyrimidin-4-amine. HPLC purity: 96.35%; ¹H NMR (400 MHz, DMSO-d₆) δ: 10.14 (s, 1H), 8.16 (s, 1H), 8.03 (d, J=8.4 Hz, 2H), 7.94-7.80 (m, 3H), 7.66 (d, J=8.4 Hz, 1H), 6.78 (s, 1H), 4.05 (s, 3H), 3.84 (q, J=7.9, 6.2 Hz, 4H), 3.76 (t, J=4.7 Hz, 4H), 3.20-3.16 (m, 1H), 2.60 (s, 3H), 1.35 (d, J=6.9 Hz, 6H).LCMS Calculated for C₂₈H₃₁N₉O (free base): 509.27; Observed: 510.45 (M+1).

4-((6-(1,3-dimethyl-1H-indazol-6-yl)-2-morpholinopyrimidin-4-yl)amino) benzamide: The title compound was obtained as a side product in the reaction carried out for N-(4-(5-(tert-butyl)-4H-1,2,4-triazol-3-yl)phenyl)-6-(1,3-dimethyl-1H-indazol-6-yl)-2-morpholinopyrimidin-4-amine. HPLC purity: 98.07%; ¹H NMR (400 MHz, DMSO-d₆) δ: 9.68 (s, 1H), 8.16 (s, 1H), 7.90-7.68 (m, 7H), 7.16 (s, 1H), 6.75 (s, 1H), 4.04 (s, 3H), 3.82 (q, J=6.7, 5.7 Hz, 4H), 3.74 (t, J=4.7 Hz, 4H), 2.52 (s, 3H); LCMS Calculated for C₂₄H₂₅N₇O₂: 443.21; Observed: 444.20 (M+1).

### Example 133

### Synthesis of N-(4-chlorophenyl)-5-fluoro-6-(1-methyl-3-(2-morpholinoethyl)-1H-indazol-6-yl)-2-morpholinopyrimidin-4-amine

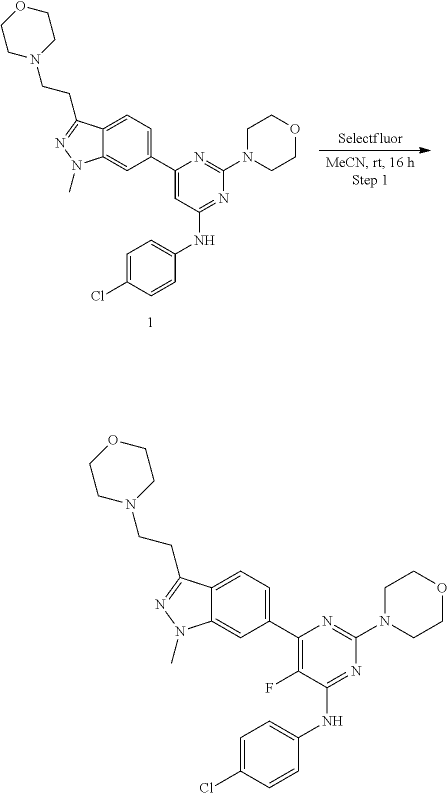 [SMILES: Cn1nc(CCN2CCOCC2)c2ccc(-c3cc(Nc4ccc(Cl)cc4)nc(N4CCOCC4)n3)cc21 ; "Selectfluor" ; Cn1nc(CCN2CCOCC2)c2ccc(-c3nc(N4CCOCC4)nc(Nc4ccc(Cl)cc4)c3F)cc21]

To a stirred solution of compound 1 (0.05 g, 1 eq) in acetonitrile (3 mL), selectfluor (0.033 g, 1 eq) was added at 0° C. The resulting reaction mixture was stirred at room temperature for 16 h. The progress of the reaction was monitored by TLC. After completion of the reaction, the reaction mixture was quenched with saturated sodium bicarbonate solution and extracted with ethyl acetate (3×20 mL). Combined organic extracts were washed with brine, dried over anhydrous sodium sulfate and evaporated under reduced pressure. The crude product was purified by preparative HPLC to afford the title compound. HPLC purity: 97.52%; ¹H NMR (400 MHz, MeOD) δ: 8.15 (s, 1H), 7.81 (s, 2H), 7.75-7.68 (m, 2H), 7.35-7.24 (m, 2H), 4.05 (s, 3H), 3.77-3.65 (m, 12H), 3.20 (dd, J=9.1, 6.8 Hz, 2H), 2.83 (t, J=8.0 Hz, 2H), 2.60 (t, J=4.6 Hz, 4H); LCMS Calculated for C₂₈H₃₁ClFN₇O₂: 551.22; Observed: 552.35 (M+1).

### Example 134

### Synthesis of 1-methyl-N-(6-(1-methyl-3-(2-morpholinoethyl)-1H-indazol-6-yl)-2-morpholinopyrimidin-4-yl)-1H-indol-6-amine

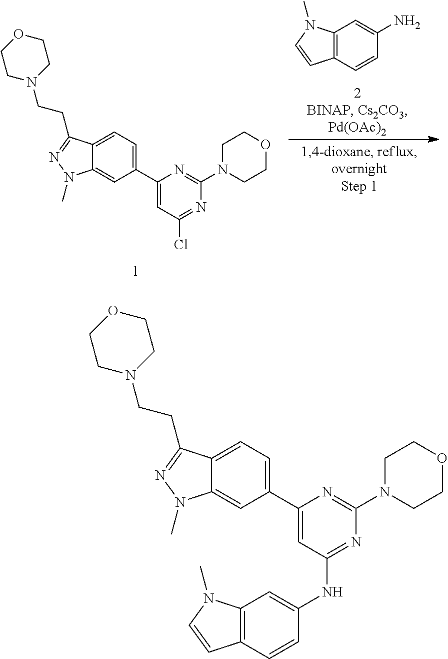

The title compound has been synthesized by following the General procedure for Buchwald Coupling described above using chloro compound 1 and compound 2.
HPLC purity: 94.11%; ¹H NMR (400 MHz, DMSO-d₆) δ: 9.40 (s, 1H), 8.17 (d, J=13.2 Hz, 2H), 7.84 (d, J=8.5 Hz, 1H), 7.71 (dd, J=8.5, 1.4 Hz, 1H), 7.60-7.43 (m, 1H), 7.24 (d, J=3.1 Hz, 1H), 7.04 (dd, J=8.4, 1.9 Hz, 1H), 6.72 (s, 1H), 6.35 (d, J=3.1 Hz, 1H), 4.02 (s, 3H), 3.87 (t, J=4.8 Hz, 4H), 3.80-3.73 (m, 8H), 3.59 (t, J=4.7 Hz, 4H), 3.09 (t, J=7.9 Hz, 2H), 2.75-2.66 (m, 2H), 2.47 (s, 3H); LCMS Calculated for C₃₁H₃₆N₈O₂: 552.30; Observed: 553.55 (M+1).

### Example 135

### Synthesis of N-(6-(1-methyl-3-(2-morpholinoethyl)-1H-indazol-6-yl)-2-morpholinopyrimidin-4-yl)-1H-indol-6-amine

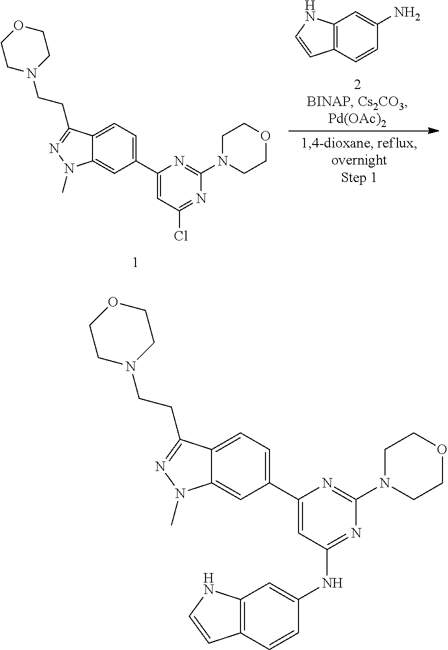

The title compound has been synthesized by following the General procedure for Buchwald Coupling described above using chloro compound 1 and amino compound 2. HPLC purity: 99.78%: ¹H NMR (400 MHz, DMSO-d₆) δ: 11.00 (s, 1H), 9.28 (s, 1H), 8.14 (s, 1H), 8.02 (s, 1H), 7.83 (d, J=8.5 Hz, 1H), 7.69 (d, J=8.5 Hz, 1H), 7.45 (d, J=8.4 Hz, 1H), 7.26 (t, J=2.7 Hz, 1H), 7.09 (dd, J=8.5, 1.9 Hz, 1H), 6.70 (s, 1H), 6.35 (d, J=2.9 Hz, 1H), 4.04 (s, 3H), 3.84 (t, J=4.7 Hz, 4H), 3.74 (t, J=4.7 Hz, 4H), 3.59 (t, J=4.6 Hz, 4H), 3.09 (t, J=7.8 Hz, 2H), 2.70 (t, J=7.9 Hz, 2H), 2.50-2.42 (m, 4H); LCMS Calculated for C₃₀H₃₄N₈O₂: 538.28; Observed: 539.45 (M+1).

### Example 136

### Synthesis of N-(6-(1-methyl-3-(2-morpholinoethyl)-1H-indazol-6-yl)-2-morpholinopyrimidin-4-yl)-1H-indazol-6-amine

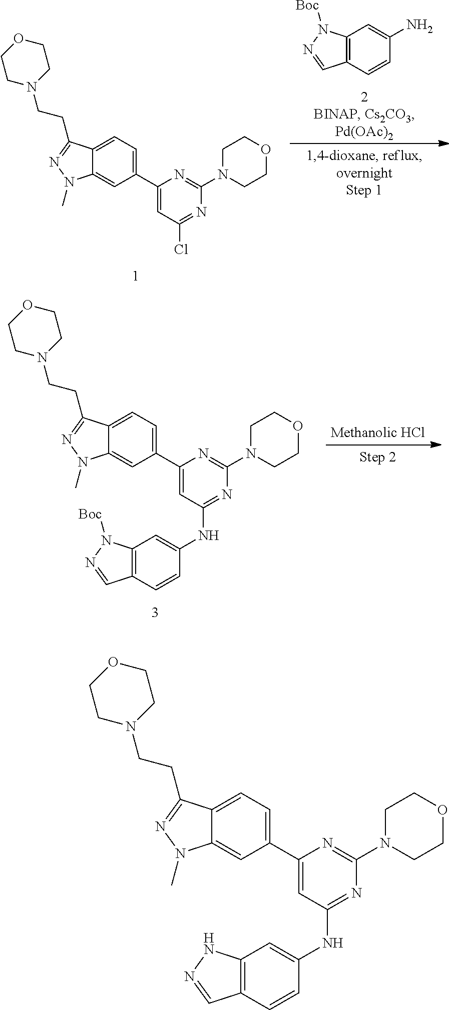 [SMILES: Cn1nc(CCN2CCOCC2)c2ccc(-c3cc(Cl)nc(N4CCOCC4)n3)cc21 ; CC(C)(C)OC(=O)n1ncc2ccc(N)cc21 ; "2BINAP, Cs2CO3, Pd(OAc)2" ; Cn1nc(CCN2CCOCC2)c2ccc(-c3cc(Nc4ccc5cnn(C(=O)OC(C)(C)C)c5c4)nc(N4CCOCC4)n3)cc21 ; "Methanolic HCl" ; Cn1nc(CCN2CCOCC2)c2ccc(-c3cc(Nc4ccc5cn[nH]c5c4)nc(N4CCOCC4)n3)cc21]

### Step 1: Synthesis of tert-butyl 6-((6-(1-methyl-3-(2-morpholinoethyl)-1H-indazol-6-yl)-2-morpholino pyrimidin-4-yl)amino)-1H-indazole-1-carboxylate (3)

The title compound has been synthesized by following the General procedure for Buchwald Coupling described above using chloro compound 1 and compound 2. LCMS (m/z): 640.15 (M+1).

### Step 2: Synthesis of N-(6-(1-methyl-3-(2-morpholinoethyl)-1H-indazol-6-yl)-2-morpholinopyrimidin-4-yl)-1H-indazol-6-amine

To a stirred solution of compound 3 (0.04 g, 1 eq) in methanol (2 mL), methanolic HCl (1 mL) was added and the reaction mixture was stirred at room temperature for 30 min. The progress of the reaction was monitored by TLC. After completion of the reaction, the reaction mixture was evaporated under reduced pressure. The residue was washed with diethyl ether and dried under reduced pressure to afford the title compound. HPLC purity: 99.43%; ¹H NMR (400 MHz, DMSO-d₆) δ: 9.59 (s, 1H), 8.29 (s, 1H), 7.94 (s, 1H), 7.85 (d, J=8.5 Hz, 1H), 7.69 (dd, J=27.6, 8.7 Hz, 2H), 7.16 (dd, J=8.7, 1.7 Hz, 1H), 6.76 (s, 1H), 4.05 (s, 3H), 3.86 (t, J=4.6 Hz, 4H), 3.77 (t, J=4.7 Hz, 4H), 3.59 (t, J=4.6 Hz, 4H), 3.09 (t, J=7.8 Hz, 2H), 2.75-2.66 (m, 2H), 2.52-2.48 (m, 4H); LCMS Calculated for C₂₉H₃₃N₉O₂: 539.28; Observed: 540.50 (M+1).

### Example 137

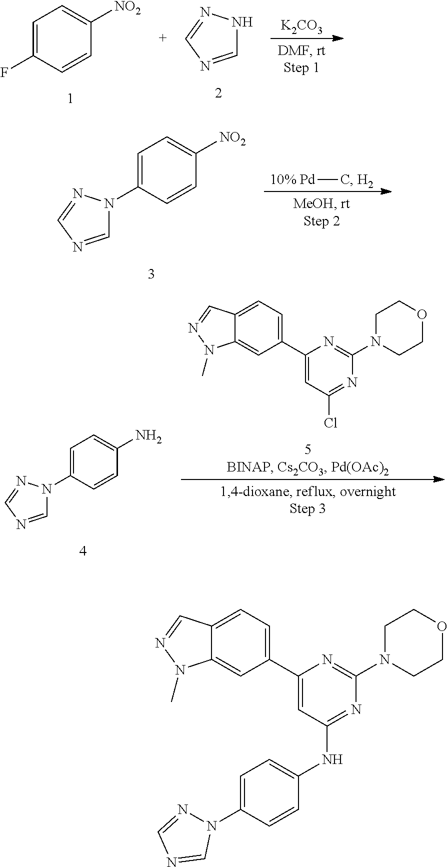

### Step 1: Synthesis of 1-(4-nitrophenyl)-1H-1,2,4-triazole (3)

To a stirred solution of compound 1 (3 g, 1 eq) in DMF (40 mL), K₂CO₃ (8.8 g, 3 eq) was added and stirred for 15 min followed by the addition of compound 2 (3.6 g, 1 eq). The reaction mixture was stirred at room temperature for 12 h. The progress of the reaction was monitored by TLC. After completion of the reaction, the reaction mixture was quenched with ice cold water. The precipitated solid was collected by filtration, washed with water and dried under reduced pressure to afford the title compound 3. LCMS (m/z): 191.00 (M+1).

### Step 2: Synthesis of 4-(1H-1,2,4-triazol-1-yl)aniline (4)

To a stirred solution of compound 3 (1.8 g, 1 eq) in methanol (10 mL), 10% Pd—C (0.5 g) was added and stirred under hydrogen atmosphere (balloon pressure) at room temperature for 16 h. The progress of the reaction was monitored by TLC. After completion of the reaction, the reaction mixture was filtered through celite. The filtrate was evaporated under reduced pressure to afford the title compound 4. LCMS (m/z): 161.00 (M+1).

### Step 3: Synthesis of N-(4-(1H-1,2,4-triazol-1-yl)phenyl)-6-(1-methyl-1H-indazol-6-yl)-2-morpholino pyrimidin-4-amine

The title compound has been synthesized by following the general procedure described above for Buchwald coupling by using corresponding chloro compound 5 and amine 4. HPLC purity: 95.28%; ¹H NMR (400 MHz, DMSO-d₆) δ: 10.80 (s, 1H), 9.29 (s, 1H), 8.25 (d, J=5.4 Hz, 2H), 8.16 (s, 1H), 7.98-7.84 (m, 5H), 7.62 (d, J=8.5 Hz, 1H), 6.85 (s, 1H), 4.15 (s, 3H), 3.87 (t, J=4.8 Hz, 4H), 3.80-3.72 (m, 4H); LCMS Calculated for C₂₄H₂₃N₉O (free base): 453.20; Observed: 454.35 (M+1).

### Examples 138-141

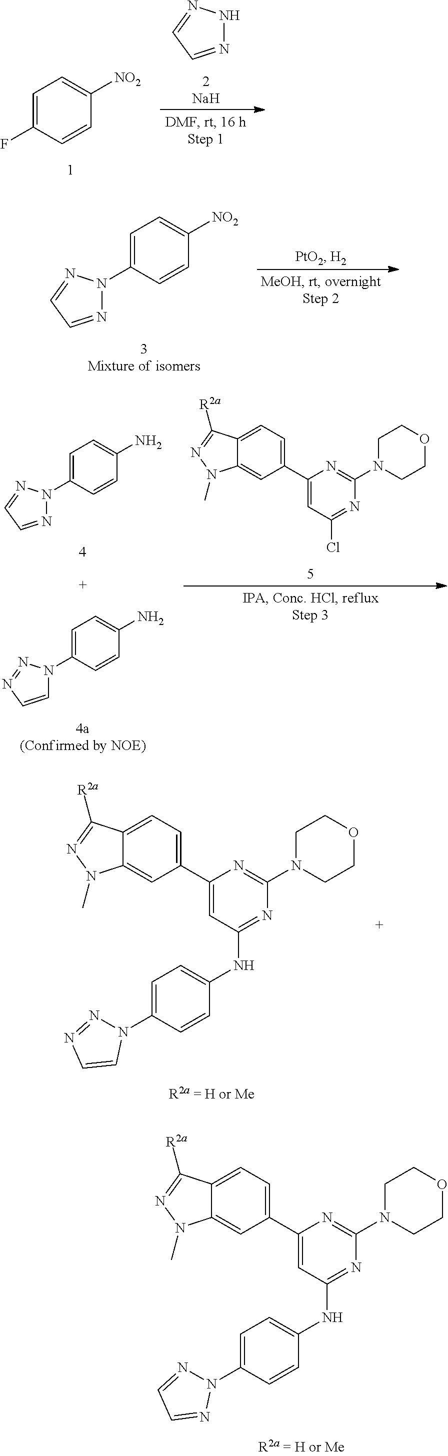

Step 1: Synthesis of 2-(4-nitrophenyl)-2H-1,2,3-triazole (3)

To a stirred solution of NaH (0.58 g, 1 eq)) in dry DMF (10 mL), compound 2 (1 g, 1 eq) was added at 0° C. and stirred for 15 min followed by the addition of compound 1 (2 g, 1 eq). The reaction mixture was stirred at room temperature for 16 h. The progress of the reaction was monitored by TLC. After completion of the reaction, the reaction mixture was quenched with ice cold water. The precipitated solid was collected by filtration, washed with water and dried under reduced pressure to afford the title compound 3 as mixture of isomers. LCMS (m/z): 190.95 (M+1).

### Step 2: Synthesis of 4-(2H-1,2,3-triazol-2-yl)aniline (4) and 4-(1H-1,2,3-triazol-1-yl)aniline (4a)

To a stirred solution of compound 3 (2 g, 1 eq) in methanol (10 mL), PtO₂ (0.19 g) was added and stirred under hydrogen atmosphere (balloon pressure) at room temperature for 16 h. The progress of the reaction was monitored by TLC. After completion of the reaction, the reaction mixture was filtered through celite. The filtrate was evaporated under reduced pressure. The crude product was purified by flash column chromatography on silica gel 100-200 mesh using 20% EtOAc-hexane to afford the title compounds 4 and 4a. Both the compounds were confirmed by NOE. LCMS (m/z): 161.05 (M+1).

### Step 3

N-(4-(2H-1,2,3-triazol-2-yl)phenyl)-6-(1-methyl-1H-indazol-6-yl)-2-morpholino pyrimidin-4-amine: The title compound has been synthesized by following the general procedure described above for displacement reaction (IPA/HCl) by using corresponding chloro compound 5 and amino compound 4. HPLC purity: 99.85%; ¹H NMR (400 MHz, DMSO-d₆) δ: 8.24 (s, 1H), 8.12 (d, J=15.7 Hz, 3H), 8.03 (d, J=8.6 Hz, 2H), 7.90 (d, J=8.9 Hz, 4H), 7.70 (d, J=8.4 Hz, 1H), 6.78 (s, 1H), 4.15 (s, 3H), 3.85 (t, J=4.6 Hz, 4H), 3.76 (t, J=4.6 Hz, 4H); LCMS Calculated for C₂₄H₂₃N₉O (free base): 453.20; Observed: 454.00 (M+1).

N-(4-(2H-1,2,3-triazol-2-yl)phenyl)-6-(1,3-dimethyl-1H-indazol-6-yl)-2-morpholino pyrimidin-4-amine: The title compound has been synthesized by following the general procedure described above for displacement reaction (IPA/HCl) by using corresponding chloro compound 5 and amino compound 4. HPLC purity: 96.63%; ¹H NMR (400 MHz, DMSO-d₆) δ: 10.24 (s, 1H), 8.16 (s, 1H), 8.13-8.00 (m, 3H), 7.94-7.81 (m, 4H), 7.64 (d, J=8.5 Hz, 1H), 6.77 (s, 1H), 4.05 (s, 3H), 3.85 (t, J=4.6 Hz, 4H), 3.76 (t, J=4.7 Hz, 4H); LCMS Calculated for C₂₅H₂₅N₉O (free base): 467.22; Observed: 468.00 (M+1).

N-(4-(1H-1,2,3-triazol-1-yl)phenyl)-6-(1-methyl-1H-indazol-6-₃71)-2-morpholino pyrimidin-4-amine: The title compound has been synthesized by following the general procedure described above for displacement reaction (IPA/HCl) by using corresponding chloro compound 5 and amino compound 4a. HPLC purity: 95.37%; ¹H NMR (400 MHz, DMSO-d₆) δ: 9.73 (s, 1H), 8.76 (s, 1H), 8.25 (s, 1H), 8.10 (s, 1H), 7.98-7.80 (m, 6H), 7.79 (d, J=8.6 Hz, 1H), 6.76 (s, 1H), 4.14 (s, 3H), 3.84 (t, J=4.5 Hz, 4H), 3.80-3.71 (m, 4H); LCMS Calculated for C₂₄H₂₃N₉O: 453.20; Observed: 454.30 (M+1).

N-(4-(1H-1,2,3-triazol-1-yl)phenyl)-6-(1,3-dimethyl-1H-indazol-6-yl)-2-morpholino pyrimidin-4-amine: The title compound has been synthesized by following the general procedure described above for displacement reaction (IPA/HCl) by using corresponding chloro compound 5 and amino compound 4a. HPLC purity: 98.66%; ¹H NMR (400 MHz, DMSO-d₆) δ: 9.72 (s, 1H), 8.75 (s, 1H), 8.17 (s, 1H), 7.98-7.83 (m, 5H), 7.88-7.70 (m, 2H), 6.75 (s, 1H), 4.04 (s, 3H), 3.84 (t, J=4.7 Hz, 4H), 3.74 (t, J=4.8 Hz, 4H), 2.10 (s, 3H); LCMS Calculated for C₂₅H₂₅N₉O: 467.22; Observed: 468.25 (M+1).

### Example 142

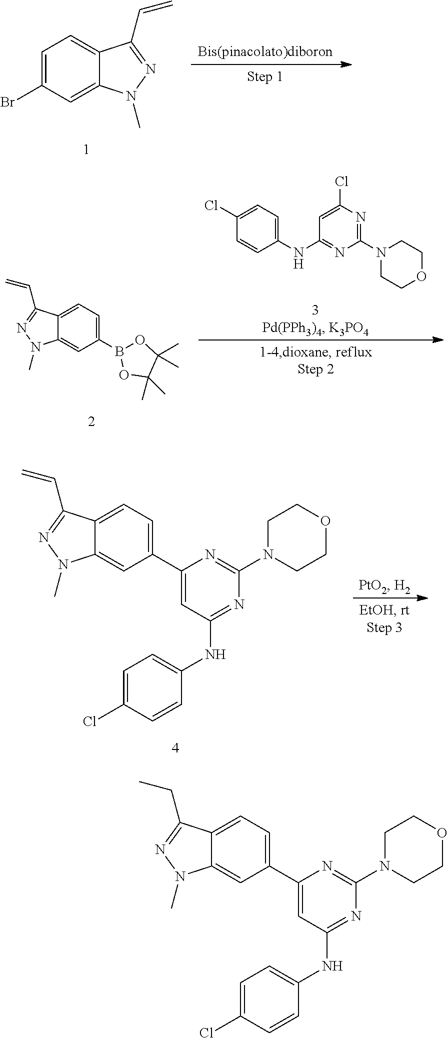 [SMILES: C=Cc1nn(C)c2cc(Br)ccc12 ; C=Cc1nn(C)c2cc(B3OC(C)(C)C(C)(C)O3)ccc12 ; Clc1ccc(Nc2cc(Cl)nc(N3CCOCC3)n2)cc1 ; C=Cc1nn(C)c2cc(-c3cc(Nc4ccc(Cl)cc4)nc(N4CCOCC4)n3)ccc12 ; CCc1nn(C)c2cc(-c3cc(Nc4ccc(Cl)cc4)nc(N4CCOCC4)n3)ccc12]

### Step 1: Synthesis of 1-methyl-6-(4,4,5,5-tetramethyl-1,3,2-dioxaborolan-2-yl)-3-vinyl-1H-indazole (2)

The title compound (crude product) has been synthesized by following the general procedure described above for Boronate ester preparation by using bromo compound 1 and Bis (pinacolato)diboron. LCMS (m/z): 285 (M+1).

### Step 2: Synthesis of N-(4-chlorophenyl)-6-(1-methyl-3-vinyl-1H-indazol-6-yl)-2-morpholinopyrimidin-4-amine (4)

The title compound has been synthesized by following the general procedure described above for Suzuki coupling by using compound 3 and Boronate ester 2. HPLC purity: 95.58%; ¹H NMR (400 MHz, DMSO-d₆) δ: 9.57 (s, 1H), 8.23 (s, 1H), 8.09 (d, J=8.5 Hz, 1H), 7.81 (dd, J=8.5, 1.4 Hz, 1H), 7.76-7.68 (m, 2H), 7.42-7.33 (m, 2H), 7.02 (dd, J=18.0, 11.4 Hz, 1H), 6.71 (s, 1H), 6.10 (d, J=18.0 Hz, 1H), 5.49 (d, J=11.5 Hz, 1H), 4.11 (s, 3H), 3.84-3.69 (m, 8H); LCMS Calculated for C₂₄H₂₃ClN₆O: 446.16; Observed: 447.15 (M+1).

### Step 3: Synthesis of N-(4-chlorophenyl)-6-(3-ethyl-1-methyl-1H-indazol-6-yl)-2-morpholinopyrimidin-4-amine

To a stirred solution of compound 4 (0.15 g, 1 eq) in ethanol (10 mL), PtO₂ (30 mg) was added and stirred under hydrogen atmosphere (balloon pressure) at room temperature for 16 h. The progress of the reaction was monitored by TLC. After completion of the reaction, the reaction mixture was filtered through celite. The filtrate was evaporated under reduced pressure. The crude product was purified by preparative HPLC to afford the desired product. HPLC purity: 99.43%; ¹H NMR (400 MHz, DMSO-d₆) δ: 9.54 (s, 1H), 8.15 (s, 1H), 7.82 (d, J=8.4 Hz, 1H), 7.71 (dd, J=8.6, 4.1 Hz, 3H), 7.37 (d, J=8.5 Hz, 2H), 6.69 (s, 1H), 4.04 (s, 3H), 3.82-3.70 (m, 8H), 2.94 (q, J=7.6 Hz, 2H), 1.33 (t, J=7.6 Hz, 3H); LCMS Calculated for C₂₄H₂₅ClN₆O: 448.18; Observed: 449.30 (M+1).

### Examples 143-144

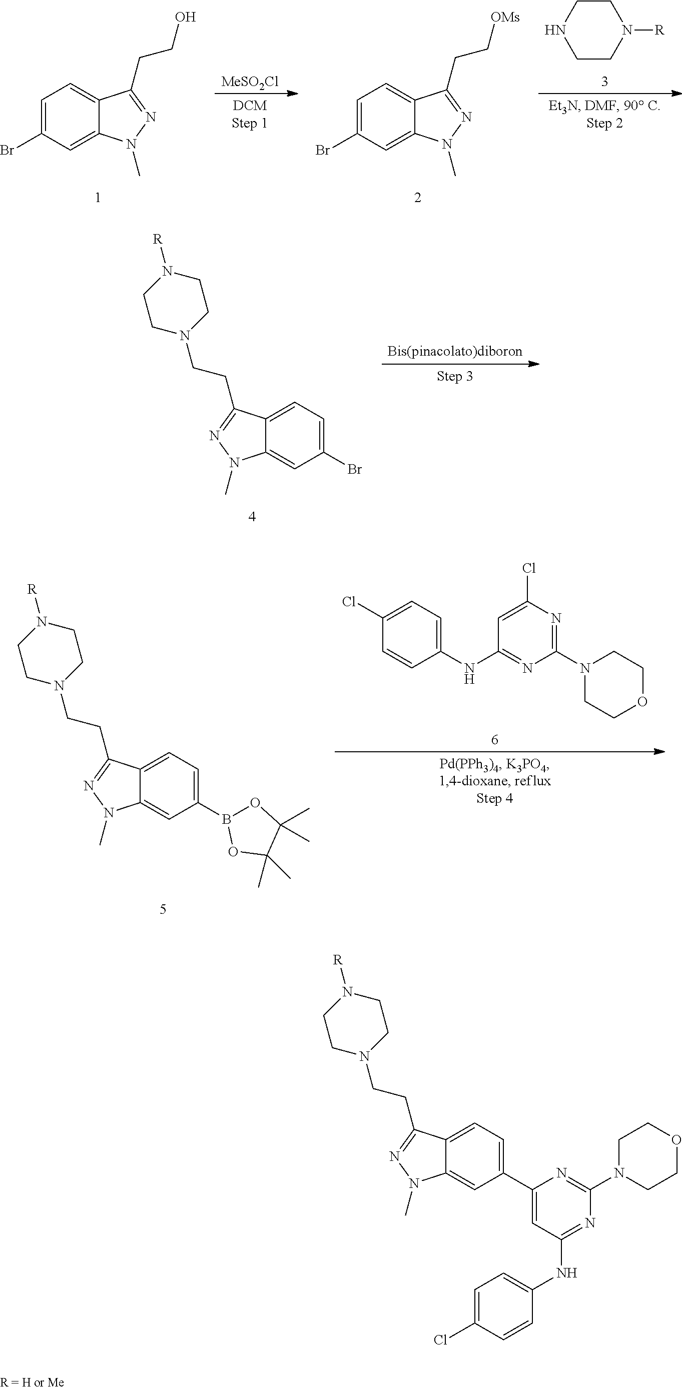

### Step 1: Synthesis of 2-(6-bromo-1-methyl-1H-indazol-3-yl) ethyl methanesulfonate (2)

To a stirred solution of compound 1(2.3 g, leq) in DCM (50 mL), TEA (2.52 mL, 2 eq) was added and stirred for 15 min followed by the slow addition of mesyl chloride (1.04 mL, 1.5 eq) at 0° C. The reaction mixture was stirred at rt for 1 h. The progress of the reaction was monitored by TLC. After completion of the reaction, the reaction mixture was diluted with water, extracted with ethyl acetate (3×25 mL). The combined organic extracts were washed with brine, dried over anhydrous sodium sulfate and evaporated under reduced pressure to afford compound 2. LCMS (m/z): 334 (M+1).

### Step 2

6-bromo-1-methyl-3-(2-(4-methylpiperazin-1-yl) ethyl)-1H-indazole (4): To a stirred solution of compound 3 (0.45 g, 1 eq)in DMF (50 mL), TEA (0.84 mL, 2 eq) was added and stirred for 15 min followed by the addition of solution of compound 2 (1 g, 1 eq). The reaction mixture was stirred at 90° C. for 1 h. The progress of the reaction was monitored by TLC. After completion of the reaction, the reaction mixture was diluted with water and extracted with ethyl acetate (3×25 mL). Combined organic extracts were washed with brine, dried over anhydrous sodium sulfate and evaporated under reduced pressure. The crude product was purified by column chromatography on silica gel 100-200 mesh using 20% EtOAc-hexane to afford title compound 4. LCMS (m/z): 339.05 (M+1).

6-bromo-1-methyl-3-(2-(piperazin-1-yl) ethyl)-1H-indazole (2): To a stirred solution of compound 3 (0.388 g, 1 eq)in DMF (50 mL), TEA (0.84 mL, 2 eq) was added and stirred for 15 min followed by the addition of solution of compound 2 (1 g, 1 eq). The reaction mixture was stirred at90° C. for 1 h. The progress of the reaction was monitored by TLC. After completion of the reaction, the reaction mixture was diluted with water and extracted with ethyl acetate (3×25 mL). Combined organic extracts were washed with brine, dried over anhydrous sodium sulfate and evaporated under reduced pressure. The crude product was purified by column chromatography on silica gel 100-200 mesh using 20% EtOAc-hexane to afford title compound 4. LCMS (m/z): 324 (M+1).

### Step 3

1-methyl-3-(2-(4-methylpiperazin-1-yl) ethyl)-6-(4,4,5,5-tetramethyl-1,3,2-dioxaborolan-2-yl)-1H-indazole (5):The title compound has been synthesized by following the general procedure described above for Suzuki coupling by using bromo compound 4 and Boronate ester. LCMS (m/z): 385 (M+1).

1-methyl-3-(2-(piperazin-1-yl) ethyl)-6-(4, 4, 5, 5-tetramethyl-1, 3, 2-dioxaborolan-2-yl)-1H-indazole(5): The title compound has been synthesized by following the general procedure described above for Suzuki coupling by using bromo compound 4 and Boronate ester. LCMS (m/z): 371 (M+1).

### Step 4

N-(4-chlorophenyl)-6-(1-methyl-3-(2-(4-methylpiperazin-1-yl)ethyl)-1H-indazol-6-yl)-2-morpholinopyrimidin-4-amine: The title compound has been synthesized by following the general procedure described above for Buchwald coupling by using chloro compound 6 and amine compound 5. ¹H NMR (400 MHz, DMSO-d₆) δ 9.59 (s, 1H), 8.19 (s,1H), 7.81-7.69 (m, 1H), 7.75-7.67 (m, 3H), 7.41-7.33 (m, 2H), 6.90(s, 1H), 4.04 (s, 3H), 3.80 (t, J=4.7 Hz, 4H), 3.76-3.69 (m, 4H), 3.07 (t, J=7.9 Hz, 2H), 2.73-2.64 (m, 4H), 2.39-2.25 (m, 6H), 2.16 (s, 3H); HPLC purity: 99.57%; LCMS Calculated for C₂₉H₃₅ClN₈O (free base): 547.09:Observed: 547.35 (M+1).

N-(4-chlorophenyl)-6-(1-methyl-3-(2-(piperazin-1-yl)ethyl)-1H-indazol-6-yl)-2-morpholinopyrimidin-4-amine: The title compound has been synthesized by following the general procedure described above for Buchwald coupling by using chloro compound 6 and amine compound 5. ¹H NMR (400 MHz, DMSO-d₆) δ 9.58(s,1H), 7.75-7.67 (m, 2H), 8.19(s, 1H), 7.85 (d, J=8 Hz, 1H),7.72-7.69(m, 3H), 7.41-7.38 (m, 2H), 6.85 (s, 1H), 4.04 (s, 1H), 3.83-3.76 (m, 4H), 3.72 (t, J=4.6 Hz, 4H), 3.07 (t, J=7.5 Hz, 2H), 3.00 (s, 2H), 2.73-2.61 (m, 6H), 2.35-2.30 (m, 4H); HPLC purity: 94.69%; LCMS Calculated for C₂₈H₃₃ClN₈O (free base): 533.07; Observed: 533.55 (M+1).

### Examples 145-148

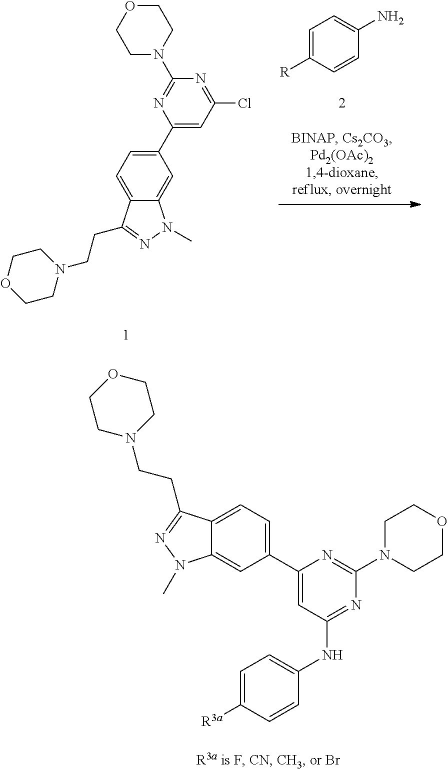

N-(4-fluorophenyl)-6-(1-methyl-3-(2-morpholinoethyl)-1H-indazol-6-yl)-2-morpholinopyrimidin-4-amine: The title compound has been synthesized by following the general procedure described above for Buchwald coupling by using chloro compound 1 and amine compound 2. ¹H NMR (400 MHz, CD₃OD) δ 8.10 (s, 1H), 8.03 (d, J=8.4 Hz, 1H), 7.64 (s, 2H), 7.56 (d, J=8.4 Hz, 1H), 7.20 (t, J=8.6 Hz, 2H), 6.57 (s, 1H), 4.14 (d, J=22.0 Hz, 5H), 3.90-3.81 (m, 12H), 3.76-3.64 (m, 4H), 3.55 (t, J=7.6 Hz, 2H), 3.21-3.19(m, 2H); HPLC purity: 97.32%; LCMS Calculated for C₂₈H₃₂FN₇O₂ (free base): 517.60; Observed: 518.40 (M+1).

N-(4-bromophenyl)-6-(1-methyl-3-(2-morpholinoethyl)-1H-indazol-6-yl)-2-morpholinopyrimidin-4-amine: The title compound has been synthesized by following the general procedure described above for Buchwald coupling by using chloro compound 1 and amine compound 2. ¹H NMR (400 MHz, DMSO-d₆) δ 9.58 (s, 1H), 8.18 (s, 1H), 7.75-7.63 (m, 1H),7.68-7.6 (m, 3H) 7.46-7.42 (m, 2H), 6.65 (s, 1H), 4.04 (s, 3H), 3.80 (t, J=4.6 Hz, 4H), 3.72 (t, J=4.6 Hz, 4H), 3.59-3.57(m, 4H), 3.09 (t, J=7.9 Hz, 2H), 2.75-2.66 (m, 2H), 2.42-2.39 (m, 4H); HPLC purity: 98.62%; LCMS Calculated for C₂₈H₃₂BrN₇O₂ (free base): 578.50; Observed: 580.0(M+1).

6-(1-methyl-3-(2-morpholinoethyl)-1H-indazol-6-yl)-2-morpholino-N-(p-tolyl)pyrimidin-4-amine: The title compound has been synthesized by following the general procedure described above for Buchwald coupling by using chloro compound 1 and amine compound 2. ¹H NMR (400 MHz, DMSO-d₆) δ 9.30 (s, 1H), 8.14 (s, 1H), 7.83 (d, J=8.5 Hz, 1H), 7.55 (d, J=8.1 Hz, 3H), 7.13 (d, J=8.0 Hz, 2H), 6.66 (s, 1H), 4.04 (s, 3H), 3.79 (t, J=4.6 Hz, 4H), 3.75-3.68 (m, 4H), 3.59 (t, J=4.6 Hz, 4H), 3.08 (t, J=7.9 Hz, 2H), 2.70 (t, J=7.9 Hz, 2H), 2.48 (s, 4H), 2.26 (s, 3H); HPLC purity: 97.07%; LCMS Calculated for C₂₉H₃₅N₇O₂ (free base): 513.63; Observed: 514.35(M+1).

4-((6-(1-methyl-3-(2-morpholinoethyl)-1H-indazol-6-yl)-2-morpholinopyrimidin-4-yl) amino) benzonitrile : The title compound has been synthesized by following the general procedure described above for Buchwald coupling by using chloro compound 1 and amine compound 2. ¹H NMR (400 MHz, Methanol-d4) δ 8.14 (s, 1H), 8.03 (d, J=8.4 Hz, 1H), 7.89 (d, J=8.4 Hz, 2H), 7.80 (d, J=8.5 Hz, 2H), 7.60 (d, J=8.6 Hz, 1H), 6.70 (s, 1H), 4.14 (d, J=24.7 Hz, 5H), 3.97-3.81 (m, 10H), 3.76-3.64 (m, 4H), 3.60-3.47 (m, 2H). HPLC purity: 97.73%; LCMS Calculated for C₂₉H₃₂N₈O₂ (free base): 524.62; Observed: 525.35 (M+1).

### Example 149

### Synthesis of N-(4-(5-cyclopropyl-4H-1,2,4-triazol-3-yl)phenyl)-6-(1-methyl-1H-indazol-6-yl)-2-morpholinopyrimidin-4-amine

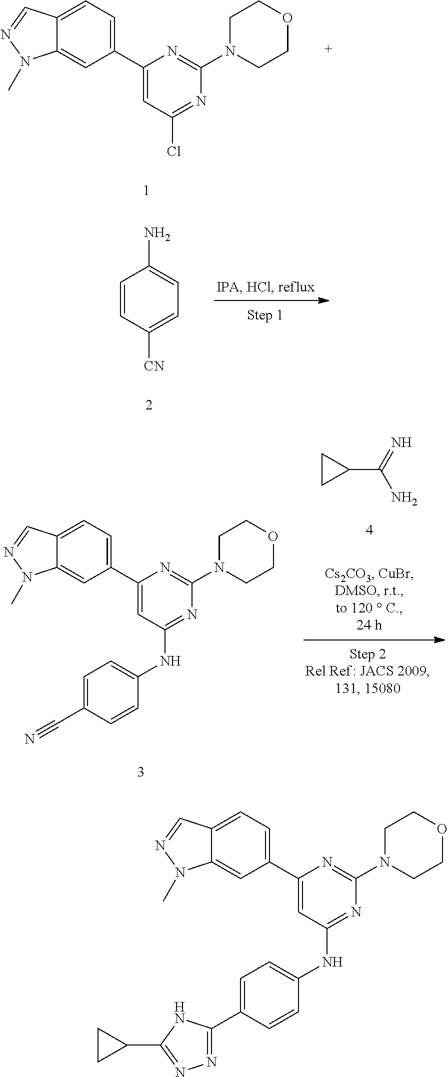 [SMILES: Cn1ncc2ccc(-c3cc(Cl)nc(N4CCOCC4)n3)cc21 ; N#Cc1ccc(N)cc1 ; N=C(N)C1CC1 ; Cn1ncc2ccc(-c3cc(Nc4ccc(C#N)cc4)nc(N4CCOCC4)n3)cc21 ; Cn1ncc2ccc(-c3cc(Nc4ccc(-c5nnc(C6CC6)[nH]5)cc4)nc(N4CCOCC4)n3)cc21]

### Step 1: Synthesis of4-((6-(1-methyl-1H-indazol-6-yl)-2-morpholinopyrimidin-4-yl) amino) benzonitrile (2)

The title compound has been synthesized by following the general procedure described above for displacement reaction (IPA/HCl) by using chloro compound 1 and amine 2. LCMS (m/z): 422.15 (M+1).

### Step-2: Synthesis of N-(4-(5-cyclopropyl-4H-1,2,4-triazol-3-yl)phenyl)-6-(1-methyl-1H-indazol-6-yl)-2-morpholinopyrimidin-4-amine

To a stirred solution of compound 3 (0.35 g, 1 eq) in DMSO (5 mL, 1 eq), copper bromide (0.0095 g, 0.05 eq), cesium carbonate (0.828 g, 2.55 eq) and compound 4 (0.153 g, 1.5 eq) was added and stirred at 100° C. for 2 day. The progress of the reaction was monitored by TLC. After completion of the reaction, the reaction mixture was quenched with sodium bicarbonate solution and extracted with ethyl acetate. The combined organic extracts were washed with brine, dried over anhydrous sodium sulfate and evaporated under reduced pressure. The crude product was purified by preparative HPLC to afford the title compound. ¹H NMR (400 MHz, DMSO-d₆) δ 10.46 (s, 1H), 8.24 (s, 1H), 8.15 (s, 1H), 8.03 (d, J=8.4 Hz, 2H), 7.89 (dd, J=13.2, 8.4 Hz, 3H), 7.67 (d, J=8.5 Hz, 1H), 6.84 (s, 1H),4.09(S, 3H), 3.85 (q, J=9.8, 7.1 Hz, 4H), 3.76 (t, J=4.8 Hz, 4H), 2.18 (td, J=8.1, 4.1 Hz, 1H), 1.12 (ddt, J=10.5, 5.4, 2.8 Hz, 4H); HPLC purity: 93.15%; LCMS Calculated for C₂₇H₂₇N₉O: 493.56; Observed: 494.25 (M+1).

### Examples 150-156

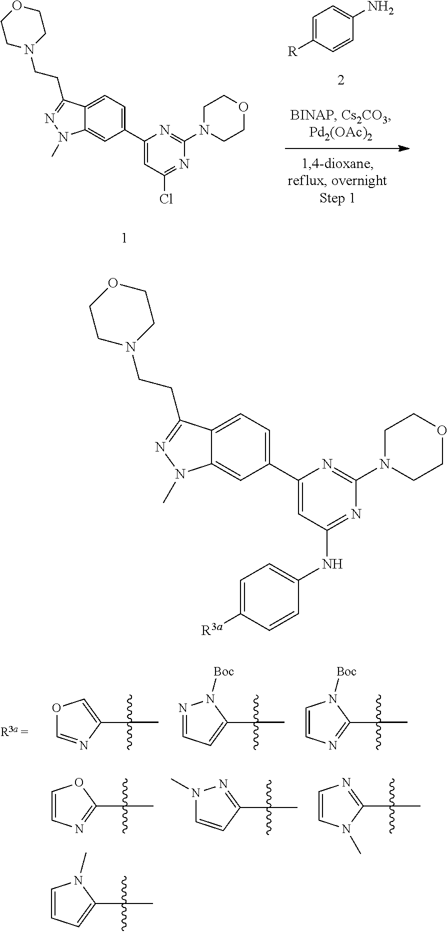

6-(1-methyl-3-(2-morpholinoethyl)-1H-indazol-6-yl)-2-morpholino-N-(4-(oxazol-4-yl) phenyl) pyrimidin-4-amine: The title compound has been synthesized by following the general procedure described above for Buchwald coupling by using chloro compound 1 and aniline. ¹H NMR (400 MHz, DMSO-d₆) δ 9.56 (s, 1H), 8.16 (s, 1H), 8.56(s, 1H), 8.51(s, 1H), 8.19(s, 1H),7.88-7.76 (m, 1H), 7.79-7.68 (m, 5H), 6.73 (s, 1H), 4.05 (s, 3H), 3.82 (q, J=6.6, 5.5 Hz, 4H), 3.74 (t, J=4.7 Hz, 4H), 3.59 (t, J=4.6 Hz, 4H), 3.09 (dd, J=10.1, 5.7 Hz, 2H), 2.71 (t, J=7.9 Hz, 2H); HPLC purity: 98.86%; LCMS Calculated for C₃₁H₃₄N₈O₃ (free base): 566.65; Observed: 567.50 (M+1).

6-(1-methyl-3-(2-morpholinoethyl)-1H-indazol-6-yl)-2-morpholino-N-(4-(oxazol-2-yl) phenyl) pyrimidin-4-amine: The title compound has been synthesized by following the general procedure described above for Buchwald coupling by using chloro compound 1 and aniline. The compound was taken in methanol (3 mL), methanol HCl(1.5 mL) was added and stirred at rt for 30 min. The reaction mixture was evaporated under reduced pressure to afford title compound as HCl salt. ¹H NMR (400 MHz, DMSO-d₆) δ 11.11 (s, 1H), 8.23 (s, 1H), 8.18 (s, 1H), 8.01-7.86 (m, 5H), 7.74 (d, J=8.4 Hz, 1H), 7.35 (s, 1H), 6.82 (s, 1H), 4.10 (s, 3H), 4.06-3.98 (m, 2H), 3.84 (d, J=11.9 Hz, 8H), 3.76 (q, J=7.3, 4.8 Hz, 6H), 3.62-3.54 (m, 2H), 3.18 (d, J=10.8 Hz, 2H); HPLC purity: 97.03%; LCMS Calculated for C₃₁H₃₄N₈O₃: 566.65; Observed: 567.50 (M+1).

N-(4-(1-methyl-1H-pyrazol-3-yl) phenyl)-6-(1-methyl-3-(2-morpholinoethyl)-1H-indazol-6-yl)-2-morpholinopyrimidin-4-amine: The title compound has been synthesized by following the general procedure described above for Buchwald coupling by using chloro compound 1 and aniline. ¹H NMR (400 MHz, DMSO-d₆) δ 9.48 (s, 1H), 8.16 (d, J=3.8 Hz, 1H), 7.84 (dt, J=8.6, 4.2 Hz, 2H), 7.71 (dd, J=13.0, 4.1 Hz, 5H), 7.57-7.41 (m, 2H), 4.05 (s, 3H), 3.90-3.77 (m, 11H), 3.77-3.70 (m, 4H), 3.09 (t, J=7.8 Hz, 2H), 2.71 (dd, J=9.0, 6.7 Hz, 2H), 2.44 (s, 4H); HPLC purity: 96.83%; LCMS Calculated for C₃₂H₃₇N₉O₂ (free base): 579.70; Observed: 580.15 (M+1).

N-(4-(1-methyl-1H-imidazol-2-yl) phenyl)-6-(1-methyl-3-(2-morpholinoethyl)-1H-indazol-6-yl)-2-morpholinopyrimidin-4-amine: The title compound has been synthesized by following the general procedure described above for Buchwald coupling by using chloro compound 1 and aniline. ¹H NMR (400 MHz, DMSO-d₆) δ 9.60 (s, 1H), 8.17 (s, 1H), 7.83 (dd, J=20.6, 8.5 Hz, 3H), 7.73 (d, J=8.4 Hz, 1H), 7.66 (d, J=8.6 Hz, 2H), 7.21 (s, 1H), 6.95 (d, J=1.2 Hz, 1H), 6.75 (s, 1H), 4.05 (s, 3H), 3.83 (t, J=4.6 Hz, 4H), 3.75 (d, J=9.2 Hz, 7H), 3.59 (t, J=4.5 Hz, 4H), 3.09 (t, J=7.8 Hz, 2H), 2.71 (t, J=7.8 Hz, 2H), 2.51 (d, J=24.0 Hz, 4H); HPLC purity: 99.84%; LCMS Calculated for C₃₂H₃₇N₉O₂ (free base): 579.70; Observed: 580.55 (M+1).

N-(4-(1-methyl-1H-pyrrol-2-yl) phenyl)-6-(1-methyl-3-(2-morpholinoethyl)-1H-indazol-6-yl)-2-morpholinopyrimidin-4-amine: The title compound has been synthesized by following the general procedure described above for Buchwald coupling by using chloro compound 1 and aniline. ¹H NMR (400 MHz, DMSO-d₆) δ 9.50 (s, 1H), 8.16 (s, 1H), 7.85 (d, J=8.5 Hz, 1H), 7.73 (td, J=8.5, 7.8, 1.8 Hz, 3H), 7.39 (d, J=8.6 Hz, 2H), 6.80 (t, J=2.2 Hz, 1H), 6.73 (s, 1H), 6.11 (dd, J=3.6, 1.9 Hz, 1H), 4.05 (s, 3H), 3.73 (t, J=4.6 Hz, 4H), 3.65 (s, 3H), 3.59 (t, J=4.6 Hz, 4H), 3.32 (s, 7H), 3.09 (dd, J=9.9, 5.9 Hz, 2H), 2.75-2.66 (m, 2H), 2.60 (s, 10H), 2.48 (s, 10H); HPLC purity: 97%; LCMS Calculated for C₃₃H₃₈N₈O₂ (free base): 578.71; Observed: 579.55 (M+1).

tert-Butyl 5-(4-((6-(1-methyl-3-(2-morpholinoethyl)-1H-indazol-6-yl)-2-morpholinopyrimidin-4-yl) amino) phenyl)-1H-pyrazole-1-carboxylate (2): The title compound has been synthesized by following the general procedure described above for Buchwald coupling by using chloro compound 1 and aniline. LCMS (m/z): 566 (M−100).

tert-Butyl 2-(4-((6-(1-methyl-3-(2-morpholinoethyl)-1H-indazol-6-yl)-2-morpholinopyrimidin-4-yl) amino) phenyl)-1H-imidazole-1-carboxylate (2): The title compound has been synthesized by following the general procedure described above for Buchwald coupling by using chloro compound 1 and aniline. LCMS (m/z): 566 (M−100).

### Examples 157-158

### Synthesis of N-(4-(1H-pyrazol-5-yl) phenyl)-6-(1-methyl-3-(2-morpholinoethyl)-1H-indazol-6-yl)-2-morpholinopyrimidin-4-amine

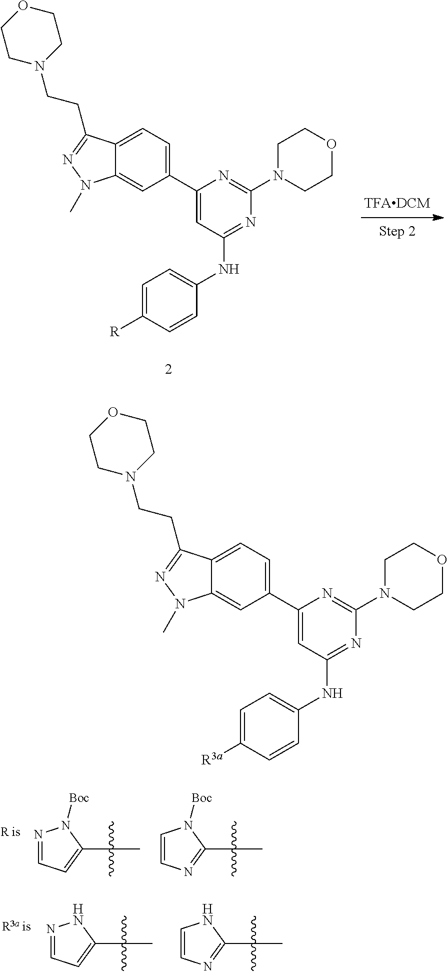

N-(4-(1H-pyrazol-5-yl) phenyl)-6-(1-methyl-3-(2-morpholinoethyl)-1H-indazol-6-yl)-2-morpholinopyrimidin-4-amine: A stirred solution of corresponding compound 2 (0.06 g, 1 eq) in TFA (0.5 mL) was stirred at rt temperature for 1 h. The progress of the reaction was monitored by TLC. After completion of the reaction, the reaction mixture was evaporated to dryness. The residue was quenched with saturated sodium bicarbonate solution and extracted with 10% MeOH-DCM (2×20 mL). Combined organic extracts were washed with brine, dried over anhydrous sodium sulfate and evaporated under reduced pressure to afford the title compound. ¹H NMR (400 MHz, DMSO-d₆) δ 12.77 (s, 1H), 9.51-9.45 (m, 1H), 8.16 (s, 1H), 7.88-7.68 (m, 7H), 6.72 (s, 1H), 6.64 (d, J=15.3 Hz, 1H), 4.05 (s, 3H), 3.87-3.69 (m, 12H), 3.09 (t, J=7.9 Hz, 2H), 2.71 (t, J=7.9 Hz, 2H), 2.47 (d, J=4.8 Hz, 4H); HPLC purity: 96.52%; LCMS Calculated for C₃₁H₃₅N₉O₂: 565.67; Observed: 471.30 (M+1).

N-(4-(1H-imidazol-2-yl)phenyl)-6-(1-methyl-3-(2-morpholinoethyl)-1H-indazol-6-yl)-2-morpholinopyrimidin-4-amine: A stirred solution of corresponding compound 2 (0.05 g, 1 eq) in DCM (2 mL), TFA (0.2 mL) was added and stirred at rt temperature for 1 h. The progress of the reaction was monitored by TLC. After completion of the reaction, the reaction mixture was evaporated to dryness. The residue was quenched with saturated sodium bicarbonate solution and extracted with 10% MeOH-DCM (2×20 mL). Combined organic extracts were washed with brine, dried over anhydrous sodium sulfate and evaporated under reduced pressure to afford the title compound. ¹H NMR (400 MHz, DMSO-d₆) δ 12.31 (s, 1H), 9.56 (s, 1H), 8.20(s, 1H), 7.87 (dd, J=15.6, 8.5 Hz, 3H), 7.74 (dd, J=17.6, 8.4 Hz, 3H), 7.09 (s, 2H), 6.73 (s, 1H), 4.05 (s, 3H), 3.82 (q, J=6.9, 5.7 Hz, 4H), 3.75 (d, J=4.7 Hz, 4H), 3.59 (t, J=4.6 Hz, 4H), 3.09 (t, J=7.8 Hz, 2H), 2.71 (t, J=7.8 Hz, 2H), 2.40-2.34 (m, 4H); HPLC purity: 98.13%; LCMS Calculated for C₃₁H₃₅N₉O₂: 565.67; Observed: 566.50 (M+1).

### Example 159

### Synthesis of N-(4-(1H-imidazol-4-yl) phenyl)-6-(1-methyl-3-(2-morpholinoethyl)-1H-indazol-6-yl)-2-morpholinopyrimidin-4-amine

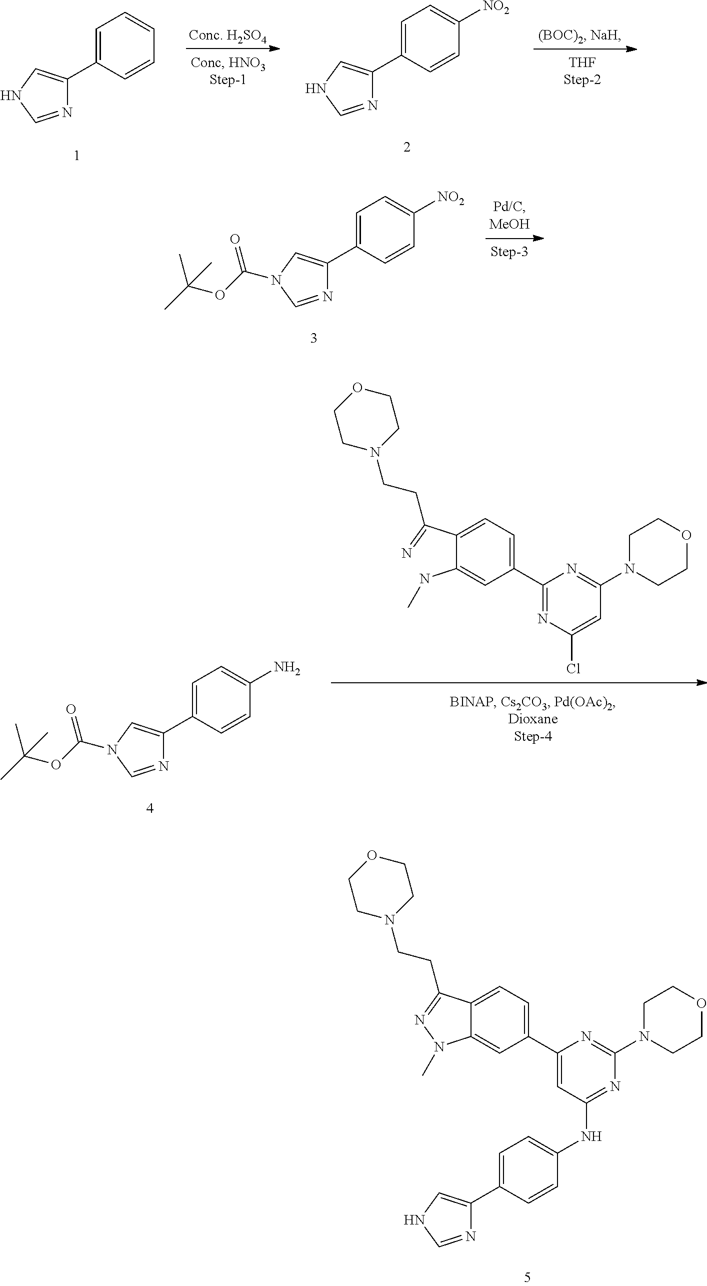 [SMILES: c1ccc(-c2c[nH]cn2)cc1 ; O=[N+]([O-])c1ccc(-c2c[nH]cn2)cc1 ; CC(C)(C)OC(=O)n1cnc(-c2ccc([N+](=O)[O-])cc2)c1 ; CC(C)(C)OC(=O)n1cnc(-c2ccc(N)cc2)c1 ; CNc1cc(-c2nc(Cl)cc(N3CCOCC3)n2)ccc1C(=N)CCN1CCOCC1 ; Cn1nc(CCN2CCOCC2)c2ccc(-c3cc(Nc4ccc(-c5c[nH]cn5)cc4)nc(N4CCOCC4)n3)cc21]

### Step-1: Synthesis of4-(4-nitrophenyl)-1H-imidazole (2)

To a stirred solution of compound 1 (1 g, 1 eq) in Conc. H₂SO₄ (4 mL), nitrating mixture (0.44 mL Conc. HNO₃+1 mL Conc. H₂SO₄) was added at 0° C. and stirred at same temperature for 2 h. The progress of the reaction was monitored by TLC. After completion of the reaction, the reaction mixture was poured onto ice water and basified to pH 9 using 2N NaOH. The precipitated solid was filtered, washed with water and dried under reduced pressure to afford the title compound 2. LCMS (m/z): 190 (M+1).

### Step-2: Synthesis of tert-butyl 4-(4-nitrophenyl)-1H-imidazole-1-carboxylate (3)

To a stirred solution of compound 2 (0.45 g, 1 eq) in THF (10 mL), sodium hydride (0.068 g, 1.2 eq) was added at 0° C. and stirred for 30 min followed by the addition of methyl iodide (0.562 g, 1.5 eq) at 0° C. and stirred at room temperature for 30 min. The progress of the reaction was monitored by TLC. After completion of the reaction, the reaction mixture was quenched with ice water and extracted with ethyl acetate (2×25 mL). Combined organic extracts were washed with brine, dried over anhydrous sodium sulfate and evaporated under reduced pressure. The residue was purified by repeated washing with pentane to afford the title compound 3. LCMS (m/z): 290 (M+1).

### Step-3: Synthesis of tert-butyl 4-(4-aminophenyl)-1H-imidazole-1-carboxylate (4)

To a stirred solution of compound 3 (0.4 g, 1 eq) in methanol (10 mL), 10% Pd/C (0.04 g) was added and stirred under hydrogen atmosphere (balloon pressure) at room temperature for 8 h. The progress of the reaction was monitored by TLC. After completion of the reaction, the reaction mixture was filtered through celite. The filtrate was evaporated under reduced pressure to afford title compound 4.

### Step-4: Synthesis of N-(4-(1H-imidazol-4-yl) phenyl)-6-(1-methyl-3-(2-morpholinoethyl)-1H-indazol-6-yl)-2-morpholinopyrimidin-4-amine

The title compound has been synthesized by following the general procedure described above for Buchwald coupling by using chloro compound Sand amine 4. ¹H NMR (400 MHz, DMSO-d₆) δ 12.22 (s, 1H), 9.43 (s, 1H), 8.16 (s, 1H), 7.85 (d, J=8.4 Hz, 1H), 7.71 (t, J=6.5 Hz, 6H), 7.48 (s, 1H), 6.71 (s, 1H), 4.05 (s, 3H), 3.82 (dt, J=9.8, 4.9 Hz, 4H), 3.77-3.68 (m, 4H), 3.65-3.58 (m, 4H), 3.11 (t, J=7.9 Hz, 2H), 2.82-2.73 (m, 2H), 2.44 (s, 4H) ; HPLC purity: 99.79%; LCMS Calculated for C₃₁H₃₅N₉O₂ (free base): 565.67; Observed: 566.55 (M +1).

### Examples 160-162

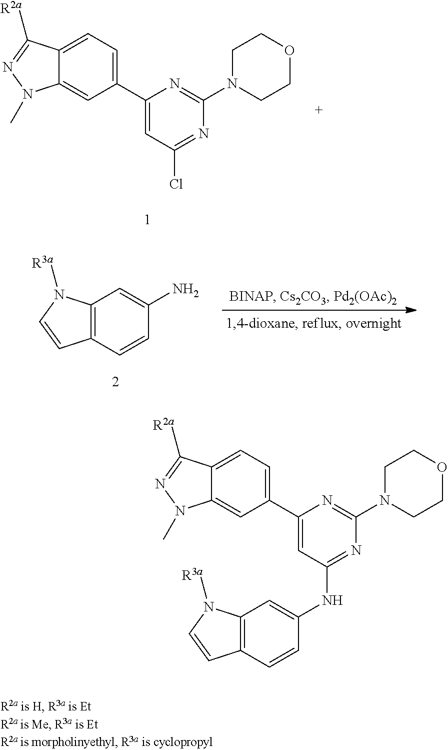

1-ethyl-N-(6-(1-methyl-1H-indazol-6-yl)-2-morpholinopyrimidin-4-yl)-1H-indol-6-amine: ¹H NMR (400 MHz, DMSO-d₆) δ 8.19 (d, J=24.0 Hz, 3H), 7.92 (d, J=8.4 Hz, 1H), 7.62 (s, 1H), 7.52 (d, J=8.4 Hz, 1H), 7.36 (d, J=3.2 Hz, 1H), 7.09 (d, J=8.5 Hz, 1H), 6.75 (s, 1H), 6.40 (d, J=3.1 Hz, 1H), 4.16 (d, J=13.0 Hz, 5H), 3.93-3.85 (m, 8H), 1.37 (t, J=7.2 Hz, 3H); HPLC purity: 99.32%; LCMS Calculated for: C₂₆H₂₇N₇O: 453.55; Observed: 454.40 (M+1).

N-(6-(1,3-dimethyl-1H-indazol-6-yl)-2-morpholinopyrimidin-4-yl)-1-ethyl-1H-indol-6-amine: The title compound has been synthesized by following the general procedure described above for Buchwald Coupling by using corresponding chloro compound land amine compound 2. ¹H NMR (400 MHz, DMSO-d₆) δ 9.39 (s, 1H), 8.27 (s, 1H), 8.15 (s, 1H), 7.79 (d, J=8.4 Hz, 1H), 7.71 (d, J=8.5 Hz, 1H), 7.46 (d, J=8.4 Hz, 1H), 7.29 (d, J=3.1 Hz, 1H), 7.00 (dd, J=8.6, 1.8 Hz, 1H), 6.73 (s, 1H), 6.36 (d, J=3.1 Hz, 1H), 4.15 (q, J=7.2 Hz, 2H), 4.03 (s, 3H), 3.87 (t, J=4.6 Hz, 8H), 2.45 (s, 3H), 1.37 (t, J=7.2 Hz, 3H); HPLC purity: 99.76%; LCMS Calculated for C₂₇H₂₉N₇O (free base): 467.57; Observed: 468 (M+1).

1-cyclopropyl-N-(6-(1-methyl-3-(2-morpholinoethyl)-1H-indazol-6-yl)-2-morpholinopyrimidin-4-yl)-1H-indol-6-amine: The title compound has been synthesized by following the general procedure described above for Buchwald Coupling by using corresponding chloro compound 1 and amine compound 2. ¹H NMR (400 MHz, DMSO-d₆) δ 11.25 (s, 1H), 8.29 (s, 1H), 8.21 (s, 1H), 7.98 (d, J=8.6 Hz, 1H), 7.67 (d, J=6.1 Hz, 1H), 7.50 (d, J=8.4 Hz, 1H), 7.28 (d, J=3.3 Hz, 1H), 7.12 (d, J=8.3 Hz, 1H), 6.79 (s, 1H), 6.35 (d, J=3.3 Hz, 1H), 4.10 (s, 3H), 4.06-3.97 (m, 2H), 3.94-3.73 (m, 10H), 3.55 (dd, J=24.1, 11.5 Hz, 6H), 3.44-3.37 (m, 1H), 3.18 (q, J=11.4, 11.0 Hz, 2H), 1.08-0.92 (m, 4H); HPLC purity: 99.48%; LCMS Calculated for C₃₃H₃₈N₈O₂ (free base): 578.71; Observed: 579.45 (M+1).

### Examples 163-164

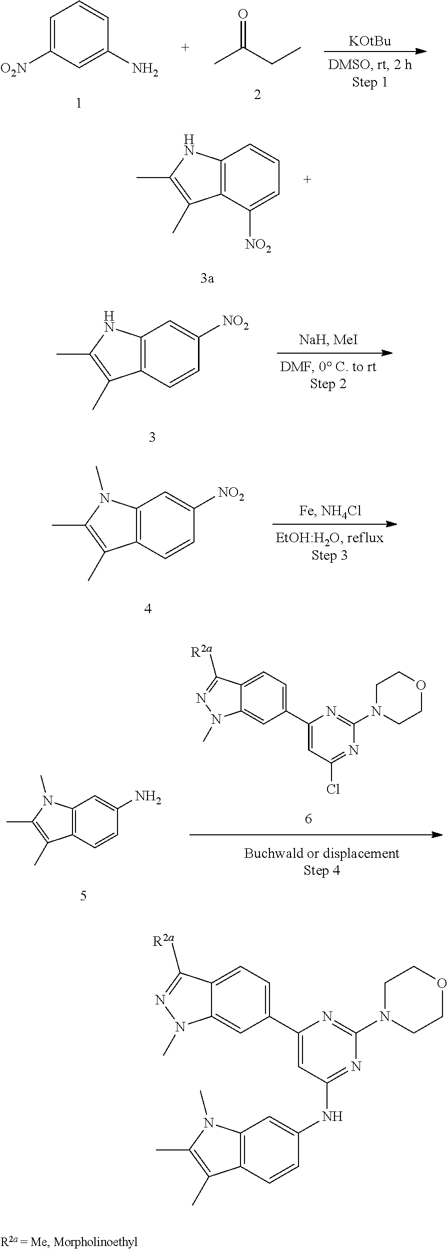

### Step-1: Synthesis of 2,3-dimethyl-4-nitro-1H-indole (3)

To a stirred solution of compound 1 (10 g, 1 eq) and compound 2 (9 mL, 1.4 eq) in DMSO (20 mL), tBuoK (19.74 g, 2.4 eq) was added. The reaction mixture was stirred at room temperature for 2 h. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 25% EtOAc-hexane to afford the title compounds 3. LCMS (m/z): 192 (M+1).

### Step-2: Synthesis of 1,2,3-trimethyl-6-nitro-1H-indole (4)

To a stirred solution of compound 3 (0.15 g, 1 eq) in DMF (5 mL), sodium hydride (0.038 g, 2 eq) was added at 0° C. and stirred for 20 min followed by the addition of methyl iodide (0.06 mL, 1.2 eq) at 0° C. and stirred at room temperature for 1 h. The progress of the reaction was monitored by TLC. After completion of the reaction, the reaction mixture was quenched with ice water and extracted with ethyl acetate (2×25 mL). Combined organic extracts were washed with brine, dried over anhydrous sodium sulfate and evaporated under reduced pressure to afford the title compound 4. LCMS (m/z): 205.1 (M+1).

### Step-3: Synthesis of 1,2,3-trimethyl-1H-indol-6-amine (5)

To a stirred solution of compound 4 (0.15 g, 1 eq) in ethanol: water (1:1, 10 mL), iron powder (0.164 g), and ammonium chloride (0.164 g) was added. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (100 mL), washed with brine, dried over anhydrous sodium sulfate and evaporated under reduced pressure to afford the title compound 5. LCMS (m/z): 175.05 (M+1).

### Step 4

N-(6-(1,3-dimethyl-1H-indazol-6-yl)-2-morpholinopyrimidin-4-yl)-1,2,3-trimethyl-1H-indol-6-amine: The title compound has been synthesized by following the general procedure described above for Buchwald coupling by using corresponding chloro compound 6 and amine 5. ¹H NMR (400 MHz, DMSO-d₆) δ 9.30 (s, 1H), 8.14 (s, 1H), 8.00 (s, 1H), 7.77 (d, J=8.4 Hz, 1H), 7.69 (d, J=8.5 Hz, 1H), 7.32 (d, J=8.4 Hz, 1H), 7.02 (dd, J=8.4, 1.9 Hz, 1H), 6.69 (s, 1H), 4.03 (s, 3H), 3.85 (t, J=4.6 Hz, 4H), 3.73 (t, J=4.8 Hz, 4H), 3.59 (s, 3H), 2.46 (s, 3H), 2.31 (s, 3H), 2.17 (s, 3H); HPLC purity: 94.25%; LCMS Calculated for C₂₈H₃₁N₇O (free base): 481.59: observed : 482.40(M+1).

1, 2, 3-trimethyl-N-(6-(1-methyl-1H-indazol-6-yl)-2-morpholinopyrimidin-4-yl)-1H-indol-6-amine: The title compound has been synthesized by following the general procedure described above for Buchwald coupling by using corresponding chloro compound 6 and amine 5. ¹H NMR (400 MHz, DMSO-d₆) δ 9.31 (s, 1H), 8.14 (s, 1H), 8.00 (s, 1H), 7.83 (d, J=8.5 Hz, 1H), 7.69 (dd, J=8.4, 1.4 Hz, 1H), 7.32 (d, J=8.4 Hz, 1H), 7.02 (dd, J=8.4, 1.8 Hz, 1H), 6.68 (s, 1H), 4.04 (s, 3H), 3.85 (t, J=4.7 Hz, 4H), 3.73 (t, J=4.8 Hz, 4H), 3.59 (h, J=4.7, 4.0 Hz, 7H), 3.30 (s, 2H), 3.08 (dd, J=8.8, 6.8 Hz, 2H), 2.49-2.39 (m, 4H), 2.31 (s, 3H), 2.17 (s, 3H); HPLC purity: 99.16%; LCMS Calculated for C₃₃H₄₀N₈O₂ (free base): 580.72; observed: 581.50(M+1).

### Example 165

### Synthesis of N-(6-(3-(3-aminopropyl)-1-methyl-1H-indazol-6-yl)-2-morpholinopyrimidin-4-yl)-1-methyl-1H-indol-6-amine

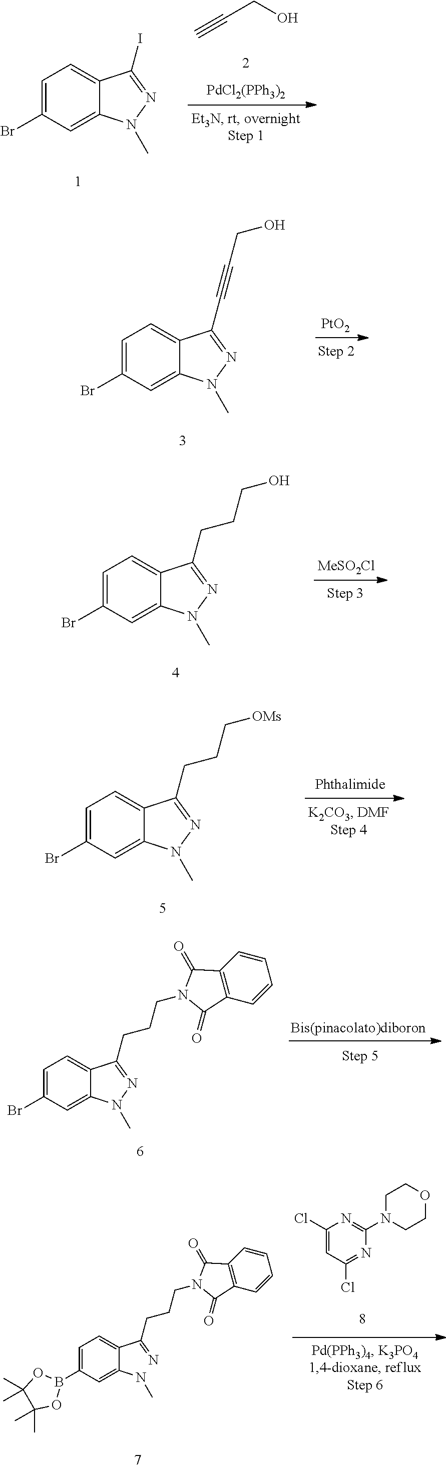

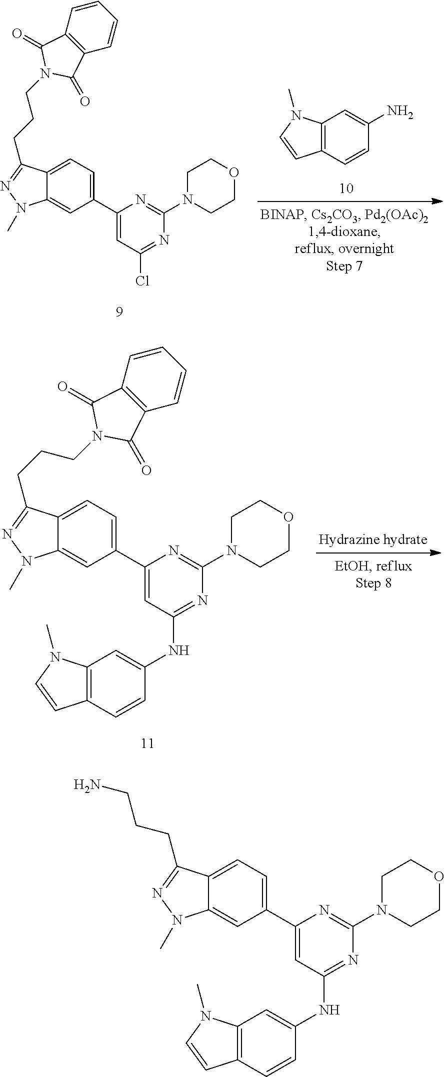 [SMILES: Cn1nc(CCCN2C(=O)c3ccccc3C2=O)c2ccc(-c3cc(Cl)nc(N4CCOCC4)n3)cc21 ; Cn1ccc2ccc(N)cc21 ; Cn1ccc2ccc(Nc3cc(-c4ccc5c(CCCN6C(=O)c7ccccc7C6=O)nn(C)c5c4)nc(N4CCOCC4)n3)cc21 ; Cn1ccc2ccc(Nc3cc(-c4ccc5c(CCCN)nn(C)c5c4)nc(N4CCOCC4)n3)cc21]

### Step-1: Synthesis of 3-(6-bromo-1-methyl-1H-indazol-3-yl)prop-2-yn-1-ol (3)

To a stirred solution of compound 1 (2 g, 1 eq) and compound 2 (0.35 mL, 1 eq) in TEA (16 mL, 20 eq), copper iodide (0.112 g, 0.1 eq) and Pd(PPh)₃Cl₂ (0.416 g, 0.1 eq)) were added and stirred at rt for 16 h. The progress of the reaction was monitored by TLC. After completion of the reaction, the reaction mixture was quenched with water and extracted with ethyl acetate. The combined organic extracts were washed with brine, dried over anhydrous sodium sulfate and evaporated under reduced pressure. The crude product was purified by column chromatography to afford the title compound 3. LCMS (m/z): 266.05 (M+1).

### Step-2: Synthesis of 3-(6-bromo-1-methyl-1H-indazol-3-yl) propan-1-ol (4)

To a stirred solution of compound 3 (1.44 g, 1 eq) in ethanol (25 mL), pt (O) (0.150 g) was added and stirred under hydrogen atmosphere (balloon pressure) at room temperature for 16 h. The progress of the reaction was monitored by TLC. After completion of the reaction, the reaction mixture was filtered through celite. The filtrate was evaporated under reduced pressure to obtain a crude residue. The crude product was purified by column chromatography to afford the title compound 4. LCMS (m/z): 270.15 (M+1).

### Step-3: Synthesis of 3-(6-bromo-1-methyl-1H-indazol-3-yl)propyl methanesulfonate (5)

To a stirred solution of compound 4(0.62 g, 1 eq) in DCM (10 mL), TEA (0.48 mL, 1.5 eq) was added and stirred for 15 min followed by the slow addition of mesyl chloride (0.21 mL, 1.2 eq) at 0° C. The reaction mixture was stirred at rt for 1 h. The progress of the reaction was monitored by TLC. After completion of the reaction, the reaction mixture was diluted with water, extracted with DCM (3×25 mL). The combined organic extracts were washed with brine, dried over anhydrous sodium sulfate and evaporated under reduced pressure to afford compound 5. LCMS (m/z): 336.1 (M+1).

### Step-4: Synthesis of 2-(3-(6-bromo-1-methyl-1H-indazol-3-yl) propyl) isoindoline-1, 3-dione (6)

To a stirred solution of compound 5 (0.6 g, 1 eq) and phthalimide (0.394 g, 1.5 eq) in DMF (10 mL), potassium carbonate (0.370 g, 1.5 eq) was added and stirred heated at 80° C. for 3h. The progress of the reaction was monitored by TLC. After completion of the reaction, the reaction mixture was quenched with water and extracted with ethyl acetate. The combined organic extracts were washed with brine, dried over anhydrous sodium sulfate and evaporated under reduced pressure to afford the title compound 6. LCMS (m/z): 399.1 (M+1).

### Step-5: Synthesis of 2-(3-(1-methyl-6-(4,4,5,5-tetramethyl-1,3,2-dioxaborolan-2-yl)-1H-indazol-3-yl)propyl)isoindoline-1,3-dione (7)

The title compound have been synthesized by following the general procedure described above for Boronate ester preparation by using bromo compound 6 and Bis (pinacolato)diboron. LCMS (m/z): 446.05 (M+1).

Step-6 : Synthesis of 2-(3-(6-(6-chloro-2-morpholinopyrimidin-4-yl)-1-methyl-1H-indazol-3-yl)propyl)isoindoline-1,3-dione (9)

The title compound has been synthesized by following the general procedure described above for Suzuki coupling by using chloro compound 8 and Boronate ester. LCMS (m/z): 517.05 (M+1).

### Step-7: Synthesis of 2-(3-(1-methyl-6-(6-((1-methyl-1H-indol-6-yl)amino)-2-morpholinopyrimidin-4-yl)-1H-indazol-3-yl)propyl)isoindoline-1,3-dione (11)

The title compound has been synthesized by following the general procedure described above for Buchwald coupling by using chloro compound 9 and amine compound 10. LCMS (m/z): 627.05 (M+1).

### Step-8: Synthesis of Synthesis of N-(6-(3-(3-aminopropyl)-1-methyl-1H-indazol-6-yl)-2-morpholinopyrimidin-4-yl)-1-methyl-1H-indol-6-amine

To a stirred solution of compound 1 (0.25 g, 1 eq) in ethanol (10 mL), hydrazine monohydrate (0.04 μL, 2 eq) was added and stirred at 95° C. for 16 h. The progress of the reaction was monitored by TLC. After completion of the reaction, the reaction mixture was quenched with water and extracted with ethyl acetate (2×25 mL). Combined organic extracts were washed with brine, dried over anhydrous sodium sulfate and evaporated under reduced pressure. The crude product was purified by column chromatography to afford the title compound 4. The compound was taken in methanol (5 mL), methanol.HCl (0.2 mL) was added and stirred at rt for 30 min. the reaction mixture was evaporated under reduced pressure and purified by washing with ether to afford title compound as HCl salt. ¹H NMR (400 MHz, DMSO-d₆) δ 8.17 (s, 1H), 8.05 (s, 4H), 7.94 (d, J=8.5 Hz, 1H), 7.60-7.49 (m, 2H), 7.31 (d, J=3.1 Hz, 1H), 7.14 (d, J =8.8 Hz, 1H), 6.80 (s, 1H), 6.40 (d, J=3.1 Hz, 1H), 4.08 (s, 3H), 4.00-3.82 (m, 4H), 3.77 (d, J=4.8 Hz, 7H), 3.04 (t, J=7.6 Hz, 2H), 2.89 (h, J=5.9 Hz, 2H), 2.06 (p, J=7.6 Hz, 2H); HPLC purity: 96.56%; LCMS Calculated for C₂₈H₃₂N₈O (free base): 496.61:observed: 497.45 (M+1).

### Examples 166-167

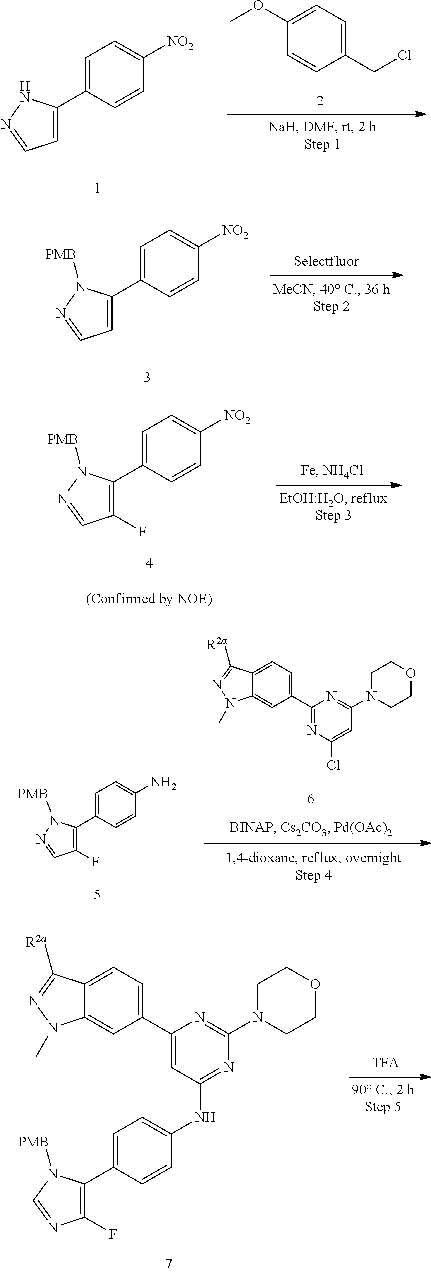

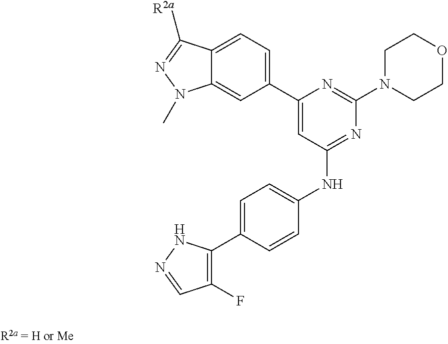

### Step 1: Synthesis of 1-(4-methoxybenzyl)-5-(4-nitrophenyl)-1H-pyrazole (3)

To a stirred solution of compound 1 (0.6 g, 1 eq) in DMF (5 mL), sodium hydride (0.182 g, 1.5 eq) was added at 0° C. and stirred for 15 min followed by the addition of compound 2 (0.742 g, 1.5 eq) at same temperature. The reaction mixture was stirred at room temperature for 1 h. The progress of the reaction was monitored by TLC. After completion of the reaction, the reaction mixture was diluted with water and extracted with ethyl acetate (2×25 mL). Combined organic extracts were washed with brine, dried over anhydrous sodium sulfate and evaporated under reduced pressure. The crude product was purified by column chromatography on silica gel 100-200 mesh using 20% EtOAc-hexane to afford title compound 3. LCMS (m/z): 310.10 (M+1).

### Step 2: Synthesis of 4-fluoro-1-(4-methoxybenzyl)-5-(4-nitrophenyl)-1H-pyrazole (4)

To a stirred solution of compound 3 (0.8 g, 1 eq) in acetonitrile (10 mL), selectfluor (1.2 g, 1.4 eq) was added at room temperature. The reaction mixture was stirred at 40° C. for 36 h. The progress of the reaction was monitored by TLC. After completion of the reaction, the reaction mixture was diluted with water and extracted with ethyl acetate (2×25 mL). Combined organic extracts were washed with brine, dried over anhydrous sodium sulfate and evaporated under reduced pressure. The crude product was purified by column chromatography on silica gel 100-200 mesh using 30% EtOAc-hexane to afford the title compound 4. Structure has been confirmed by NOE. LCMS (m/z): 328.05 (M+1).

### Step 3: Synthesis of 4-(4-fluoro-1-(4-methoxybenzyl)-1H-pyrazol-5-yl)aniline (5)

The title compound has been synthesized by following the general procedure described above for reduction using the nitro compound 4 and Fe/NH₄Cl. LCMS (m/z): 298.15 (M+1).

### Step 4

N-(4-(4-fluoro-1-(4-methoxybenzyl)-1H-pyrazol-5-yl) phenyl)-6-(1-methyl-1H-indazol-6-yl)-2-morpholinopyrimidin-4-amine (7): The title compound has been synthesized by following the general procedure described above for Buchwald Coupling by using corresponding chloro compound 6 and amine 5. LCMS (m/z): 591.45 (M+1).

6-(1,3-dimethyl-1H-indazol-6-yl)-N-(4-(4-fluoro-1-(4-methoxybenzyl)-1H-pyrazol-5-yl)phenyl)-2-morpholinopyrimidin-4-amine (7): The title compound has been synthesized by following the general procedure described above for Buchwald Coupling by using corresponding chloro compound 6 and amine 5. LCMS (m/z): 605.60 (M+1).

### Step 5

N-(4-(4-fluoro-1H-pyrazol-5-yl)phenyl)-6-(1-methyl-1H-indazol-6-yl)-2-morpholinopyrimidin-4-amine: A stirred solution of corresponding compound 7 (0.04 g, 1 eq) in TFA (5 mL) was stirred at reflux temperature for 3 h. The progress of the reaction was monitored by TLC. After completion of the reaction, the reaction mixture was evaporated to dryness. The residue was quenched with saturated sodium bicarbonate solution and extracted with 10% MeOH-DCM (2×20 mL). Combined organic extracts were washed with brine, dried over anhydrous sodium sulfate and evaporated under reduced pressure. The crude product was purified by preparative HPLC to afford the title compound. 41 NMR (400 MHz, CD3OD) δ: 8.23 (s, 1H), 8.03(s, 1H), 7.82-7.72 (m, 7H), 6.67 (s, 1H), 4.14 (s, 3H), 3.93-3.81 (m, 8H); HPLC purity: 98.07%; LCMS Calculated for C₂₅H₂₃FN₈O: 470.20; Observed: 471.30 (M+1).

6-(1,3-dimethyl-1H-indazol-6-yl)-N-(4-(4-fluoro-1H-pyrazol-5-yl)phenyl)-2-morpholinopyrimidin-4-amine: The title compound has been synthesized by following the procedure described above using corresponding compound 7 and TFA. ¹H NMR (400 MHz, CD₃OD) δ: 8.12 (s, 1H), 7.80-7.74 (m, 7H), 6.64 (s, 1H), 4.04 (s, 3H), 3.92-3.80 (m, 8H), 2.55 (s, 3H); HPLC purity: 97.97%; LCMS Calculated for C₂₆H₂₅FN₈O: 484.21; Observed: 485.35 (M+1).

### Examples 168-171

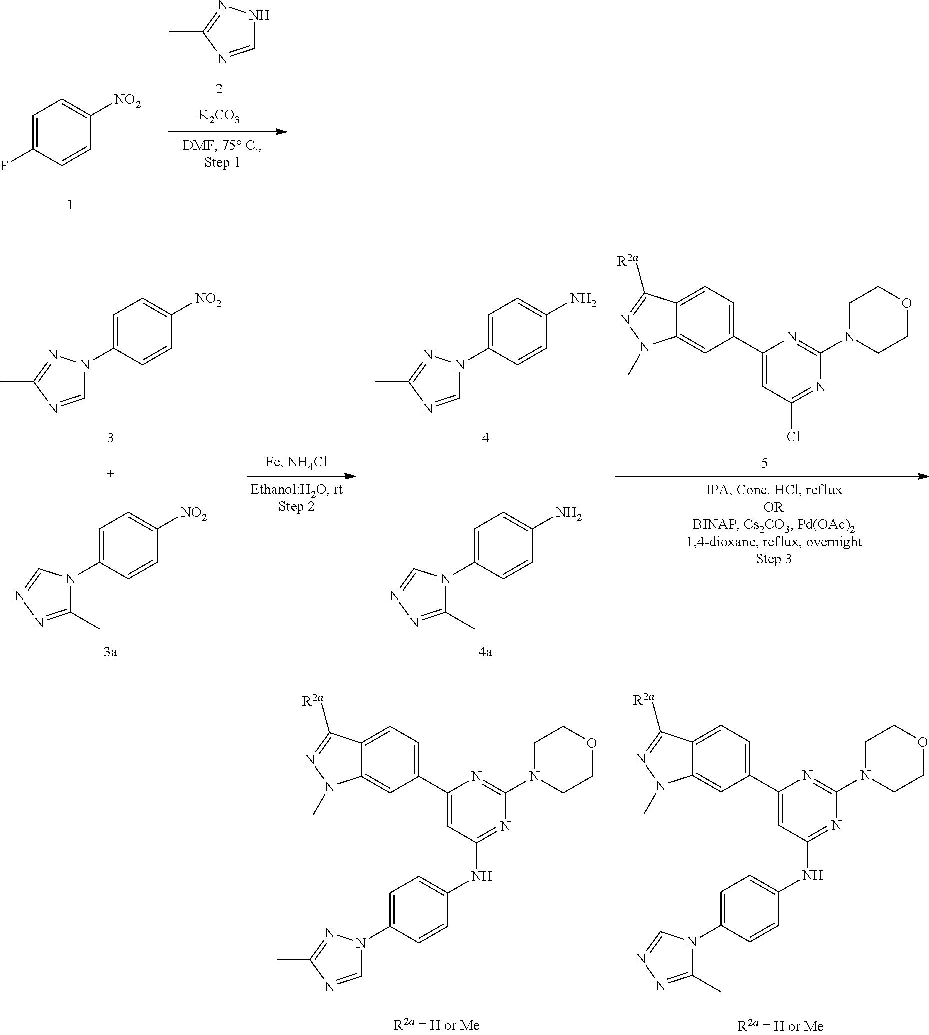

### Step 1: Synthesis of 3-methyl-1-(4-nitrophenyl)-1H-1,2,4-triazole (3) and 3-methyl-4-(4-nitrophenyl)-4H-1,2,4-triazole (3a)

To a stirred solution of compound 1 (2 g, 1 eq) in DMF (40 mL), K₂CO₃ (5.87 g, 3 eq) was added and stirred for 15 min followed by the addition of compound 2 (1.17 g, 1 eq). The reaction mixture was stirred at 75° C. for 12 h. The progress of the reaction was monitored by TLC. After completion of the reaction, the reaction mixture was quenched with ice cold water and extracted with ethyl acetate (3×20 mL). Combined organic extracts were washed with brine, dried over anhydrous sodium sulfate and evaporated under reduced pressure. The crude product was purified by column chromatography on silica gel 100-200 mesh using 50% EtOAc-hexane to afford the title compounds 3 and 3a. LCMS (m/z): 205.00 (M+1).

### Step 2

4-(3-methyl-1H-1,2,4-triazol-1-yl)aniline (4): To a stirred solution of compound 3 (1.6 g, 1 eq) in ethanol (100 mL), iron powder (2.19 g, 5 eq), water (50 mL) and ammonium chloride (2.09 g, 5 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (100 mL), washed with brine, dried over anhydrous sodium sulfate and evaporated under reduced pressure to afford the title compound 4. LCMS (m/z): 175.00 (M+1).

4-(3-methyl-4H-1,2,4-triazol-4-yl)aniline (4a): To a stirred solution of compound 3a (0.6 g, 1 eq) in ethanol (40 mL), iron powder (0.823 g, 5 eq), water (20 mL) and ammonium chloride (0.78 g, 5 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (100 mL), washed with brine, dried over anhydrous sodium sulfate and evaporated under reduced pressure to afford the title compound 4a. LCMS (m/z): 175.00 (M+1).

### Step 3

N-(4-(3-methyl-1H-1,2,4-triazol-1-yl)phenyl)-6-(1-methyl-1H-indazol-6-₃71)-2-morpholinopyrimidin-4-amine: The title compound has been synthesized by following the general procedure described above for displacement reaction (IPA/HCl) by using corresponding chloro compound 5 and amino compound 4. HPLC purity: 98.48%; ¹H NMR (400 MHz, DMSO-d₆) δ: 9.64 (s, 1H), 9.05 (s, 1H), 8.24 (s, 1H), 8.10 (s, 1H), 7.89-7.81 (m, 3H), 7.77 (dd, J=8.7, 4.6 Hz, 3H), 6.73 (s, 1H), 4.13 (s, 3H), 3.86-3.77 (m, 4H), 3.74 (t, J=4.7 Hz, 4H), 2.45 (s, 3H); LCMS Calculated for C₂₅H₂₅N₉O: 467.22; Observed: 468.30 (M+1).

6-(1,3-dimethyl-1H-indazol-6-yl)-N-(4-(3-methyl-1H-1,2,4-triazol-1-yl)phenyl)-2-morpholinopyrimidin-4-amine: The title compound has been synthesized by following the general procedure described above for Buchwald coupling by using corresponding chloro compound 5 and amine 4. HPLC purity: 98.44%; ¹H NMR (400 MHz, DMSO-d₆) δ: 10.46 (s, 1H), 9.19 (s, 1H), 8.15 (s, 1H), 7.85 (q, J=8.7 Hz, 5H), 7.59 (d, J=8.5 Hz, 1H), 6.80 (s, 1H), 4.06 (s, 3H), 3.89-3.80 (m, 4H), 3.76 (t, J=4.6 Hz, 4H), 2.97 (s, 3H), 2.38 (s, 3H); LCMS Calculated for C₂₆H₂₇N₉O (free base): 481.23; Observed: 482.30 (M+1).

6-(1-methyl-1H-indazol-6-yl)-N-(4-(3-methyl-4H-1,2,4-triazol-4-yl)phenyl)-2-morpholinopyrimidin-4-amine: The title compound has been synthesized by following the general procedure described above for Buchwald coupling by using corresponding chloro compound 5 and amino compound 4a. HPLC purity: 98.90%; ¹H NMR (400 MHz, DMSO-d₆) δ: 10.30 (s, 1H), 8.25 (s, 1H), 8.15 (d, J=7.7 Hz, 2H), 7.91 (d, J=8.4 Hz, 3H), 7.70 (d, J=8.7 Hz, 1H), 7.58 (d, J=8.4 Hz, 2H), 6.81 (s, 1H), 4.14 (s, 3H), 3.88-3.81 (m, 4H), 3.75 (t, J=4.4 Hz, 4H), 2.45 (s, 3H); LCMS Calculated for C₂₅H₂₅N₉O (free base): 467.22; Observed: 468.20 (M+1).

6-(1,3-dimethyl-1H-indazol-6-yl)-N-(4-(3-methyl-4H-1,2,4-triazol-4-yl)phenyl)-2-morpholinopyrimidin-4-amine: The title compound has been synthesized by following the general procedure described above for Buchwald coupling by using corresponding chloro compound 5 and amino compound 4a. HPLC purity: 99.31%; ¹H NMR (400 MHz, DMSO-d₆) δ: 10.44 (s, 1H), 8.17 (d, J=7.2 Hz, 2H), 7.88 (dd, J=23.1, 8.4 Hz, 3H), 7.61 (dd, J=16.5, 8.6 Hz, 3H), 6.81 (s, 1H), 4.05 (s, 3H), 3.89-3.81 (m, 4H), 3.79-3.71 (m, 4H), 2.45 (s, 6H); LCMS Calculated for C₂₆H₂₇N90 (free base): 481.23; Observed: 482.25 (M+1).

### Example 172

### Synthesis of N-(4-chlorophenyl)-6-(1-methyl-1H-pyrazolo[4,3-b]pyridin-6-yl)-2-morpholinopyrimidin-4-amine

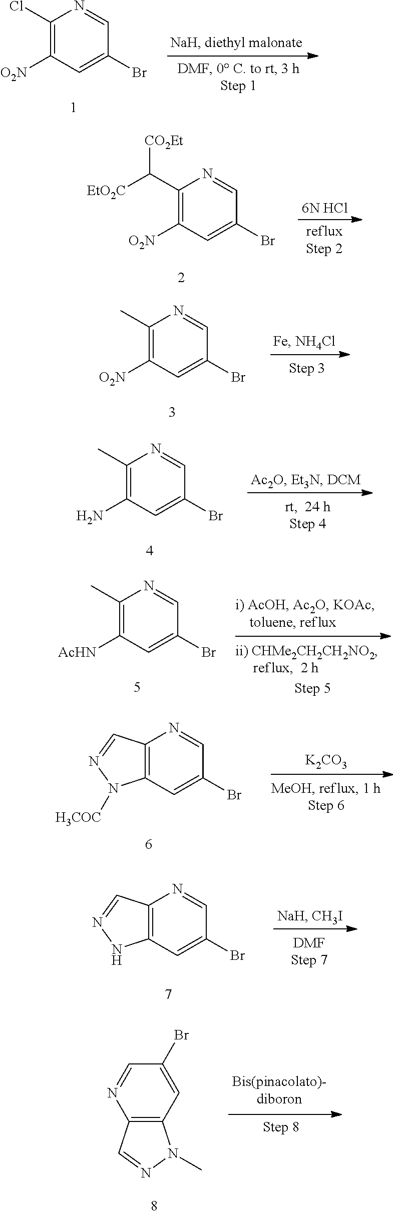

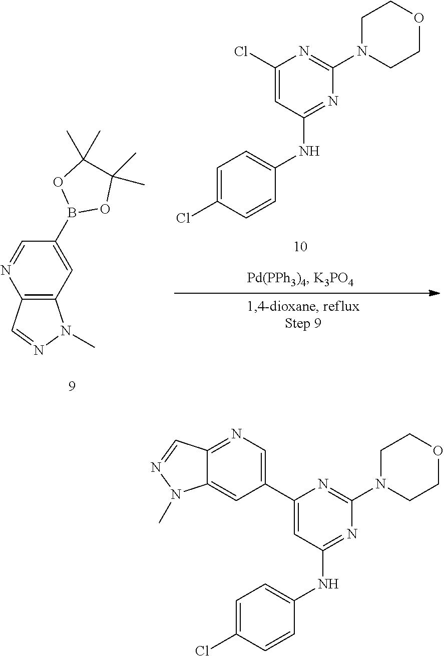 [SMILES: Cn1ncc2ncc(B3OC(C)(C)C(C)(C)O3)cc21 ; Clc1ccc(Nc2cc(Cl)nc(N3CCOCC3)n2)cc1 ; Cn1ncc2ncc(-c3cc(Nc4ccc(Cl)cc4)nc(N4CCOCC4)n3)cc21]

Step 1: Synthesis of diethyl 2-(5-bromo-3-nitropyridin-2-yl) malonate (2)

To a stirred solution of diethylmalonate (3.27 mL, 1 eq) in DMF (30 mL), NaH (0.86 g, 1.7 eq) was added portion wise at 0° C. and stirred for 15 min followed by the addition of 5-bromo-2-chloro-3-nitropyridine 1 (3 g, 1 eq).The reaction mixture was stirred at room temperature for 3 h. The progress of the reaction was monitored by TLC. After completion of the reaction, the reaction mixture was quenched with water and extracted with diethyl ether (3×50 mL). Combined organic extracts were washed with brine, dried over anhydrous sodium sulfate and evaporated under reduced pressure to afford the title compound 2. LCMS (m/z): 361.05 (M+1).

### Step 2: Synthesis of 5-bromo-2-methyl-3-nitropyridine (3)

A stirred solution of compound 2 (4.1 g, 1 eq) in 6 N HCl(30 mL) was heated to reflux for 18 h. The progress of the reaction was monitored by TLC. After completion of the reaction, reaction mixture was filtered through celite and evaporated under reduced pressure. The residue was dissolved in ethyl acetate (100 mL) and washed with brine. The organic extract was dried over anhydrous sodium sulfate and evaporated under reduced pressure to afford the title compound 3. LCMS (m/z): 218.95 (M+2).

### Step 3: Synthesis of 5-bromo-2-methylpyridin-3-amine (4)

To a stirred solution of compound 3(1.73 g, 1 eq) in ethanol (10 mL), iron powder (1.78 g, 4 eq), water (10 mL) and ammonium chloride (1.7 g,4 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (100 mL) and washed with brine. The organic extract was dried over anhydrous sodium sulfate and evaporated under reduced pressure to afford the title compound 4. LCMS (m/z): 188.90 (M+2).

### Step 4: Synthesis of N-(5-bromo-2-methylpyridin-3-yl)acetamide (5)

To a stirred solution of compound 4 (1.15 g, 1 eq) in DCM (10 mL), acetic anhydride (0.99 mL,1.7 eq) and TEA (1 mL, 2.3 eq) were added and stirred at room temperature for 24 h. The progress of the reaction was monitored by TLC. After completion of the reaction, the reaction mixture was quenched with saturated sodium bicarbonate solution and extracted with ethyl acetate (3×25 mL). Combined organic extracts were dried over anhydrous sodium sulfate and evaporated under reduced pressure to afford the title compound 5. LCMS (m/z): 231.00 (M+2).

### Step 5: Synthesis of 1-(6-bromo-1H-pyrazolo[4,3-b]pyridin-1-yl)ethanone (6)

To a stirred solution of compound 5 (1.35 g,1 eq) in toluene (15 mL), potassium acetate (1.27 g, 2.2 eq), acetic anhydride (1.67 mL, 3 eq) and acetic acid (1.68 mL, 5 eq) was heated to reflux followed by the addition of isoamyl nitrite (1 mL, 1.25 eq) and refluxed for 2 h. The progress of the reaction was monitored by TLC. After completion of the reaction, the reaction mixture was quenched with saturated sodium bicarbonate solution and extracted with ethyl acetate (2×50 mL). Combined organic extracts were dried over anhydrous sodium sulfate and evaporated under reduced pressure to afford the title compound 6. LCMS (m/z): 242.15 (M+2).

### Step 6: Synthesis of 6-bromo-1H-pyrazolo[4,3-b]pyridine (7)

To a stirred solution of compound 6 (1.25 g, 1 eq) in methanol (15 mL), K₂CO₃ (2.15 g, 3 eq) was added and heated to reflux for 1 h. The progress of the reaction was monitored by TLC. After completion of the reaction, the reaction mixture was evaporated to dryness. The residue was dissolved in water and extracted with ethyl acetate (2×25 mL). Combined organic extracts were dried over anhydrous sodium sulfate and evaporated under reduced pressure to afford the title compound 7. LCMS (m/z): 199.90 (M+1).

### Step 7: Synthesis of 6-bromo-l-methyl-1H-pyrazolo[4,3-b]pyridine (8)

To a stirred solution of compound 7 (0.95 g,1 eq) in DMF (10 mL), NaH (0.29 g, 1.5 eq) was added and stirred at room temperature for 15 min followed by the addition of methyl iodide (0.45 mL, 1.5 eq). The reaction mixture was stirred at room temperature for 1 h. The progress of the reaction was monitored by TLC. After completion of the reaction, the reaction mixture was diluted with water and extracted with ethyl acetate (3×25 mL). Combined organic extracts were dried over anhydrous sodium sulfate and evaporated under reduced pressure to afford the title compound 8. LCMS (m/z): 211.90 (M+1).

### Step 8: Synthesis of 1-methyl-6-(4,4,5,5-tetramethyl-1,3,2-dioxaborolan-2-yl)-1H-pyrazolo[4,3-b]pyridine (9)

The title compound has been synthesized by following the general procedure described above for Boronate ester preparation by using bromo compound 8 and Bis (pinacolato) diboron. LCMS (m/z): 259.90 (M+1).

### Step 9: Synthesis of N-(4-chlorophenyl)-6-(1-methyl-1H-pyrazolo[4,3-b]pyridin-6-yl)-2-morpholinopyrimidin-4-amine

The title compound has been synthesized by following the general procedure described above for Suzuki coupling by using compound 10 and Boronate ester 9. ¹H NMR (400 MHz, DMSO-d₆) δ: 9.89 (s, 1H), 9.09 (d, J=1.8 Hz, 1H), 8.69 (t, J=1.3 Hz, 1H), 8.36 (d, J=1.0 Hz, 1H), 7.77-7.68 (m, 2H), 7.44-7.35 (m, 2H), 6.77 (s, 1H), 4.18 (s, 3H), 3.82-3.73 (m, 8H); HPLC purity: 99.46%; LCMS Calculated for C₂₁H₂₀ClN₇O: 421.14; Observed: 422.20 (M+1).

### Example 173

### Synthesis of N-(4-chlorophenyl)-6-(1,3-dimethyl-1H-pyrazolo [4,3-b]pyridin-6-yl)-2-morpholinopyrimidin-4-amine

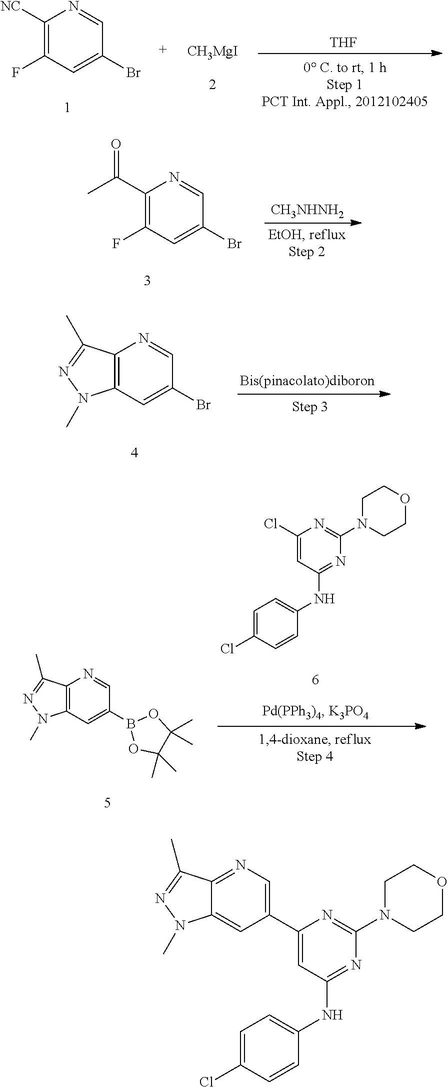

### Step 1: Synthesis of 1-(5-bromo-3-fluoropyridin-2-yl) ethan-1-one (3)

To a stirred solution of compound 1 (0.2 g, 1 eq) in THF (3 mL),methyl magnesium iodide (0.496 g, 3 eq) was added at 0° C. and stirred at room temperature for 1 h. The progress of the reaction was monitored by TLC. After completion of the reaction, the reaction mixture was quenched with saturated ammonium chloride solution and extracted with ethyl acetate (2×25 mL). Combined organic extracts were washed with brine and dried over anhydrous sodium sulfate and evaporated under reduced pressure. The crude product was purified by column chromatography on silica gel 100-200 mesh using 10% EtOAc-hexane to afford the title compound 3. LCMS (m/z): 217.95 (M+1).

### Step 2: Synthesis of 6-bromo-1,3-dimethyl-1H-pyrazolo[4,3-b]pyridine (4)

To a stirred solution of compound 3 (0.09 g, 1 eq) in ethanol (1 mL), methyl hydrazine(0.5 mL) was added and heated at 80° C. for 2 h. The progress of the reaction was monitored by TLC. After completion of the reaction, the reaction mixture was quenched with water and extracted with ethyl acetate (2×25 mL). Combined organic extracts were washed with brine, dried over anhydrous sodium sulfate and evaporated under reduced pressure. The crude product was purified by column chromatography on silica gel 100-200 mesh using 30% EtOAc-hexane to afford the title compound 4. LCMS (m/z): 227.90 (M+2).

### Step 3: Synthesis of 1,3-dimethyl-6-(4,4,5,5-tetramethyl-1,3,2-dioxaborolan-2-yl)-1H-pyrazolo[4,3-b]pyridine (5)

The title compound has been synthesized by following the general procedure described above for Boronate ester preparation by using bromo compound 4 and Bis (pinacolato) diboron. LCMS (m/z): 192.00 (M+1, boronic acid).

### Step 3: Synthesis of N-(4-chlorophenyl)-6-(1,3-dimethyl-1H-pyrazolo[4,3-b]pyridin-6-yl)-2-morpholino pyrimidin-4-amine

The title compound has been synthesized by following the general procedure described above for Suzuki coupling by using chloro compound 6 and Boronate ester 5. ¹H NMR (400 MHz, DMSO-d₆) δ: 10.06 (s, 1H), 9.00 (s, 1H), 8.64 (d, J=1.7 Hz, 1H), 7.73 (d, J=8.4 Hz, 2H), 7.40 (d, J=8.4 Hz, 2H), 6.77 (s, 1H), 4.09 (s, 3H), 3.86-3.73 (m, 8H),2.57 (s, 3H); HPLC purity: 98.62%; LCMS Calculated for C₂₂H₂₂ClN₇O: 435.16; Observed: 436.30 (M+1).

### Examples 174-175

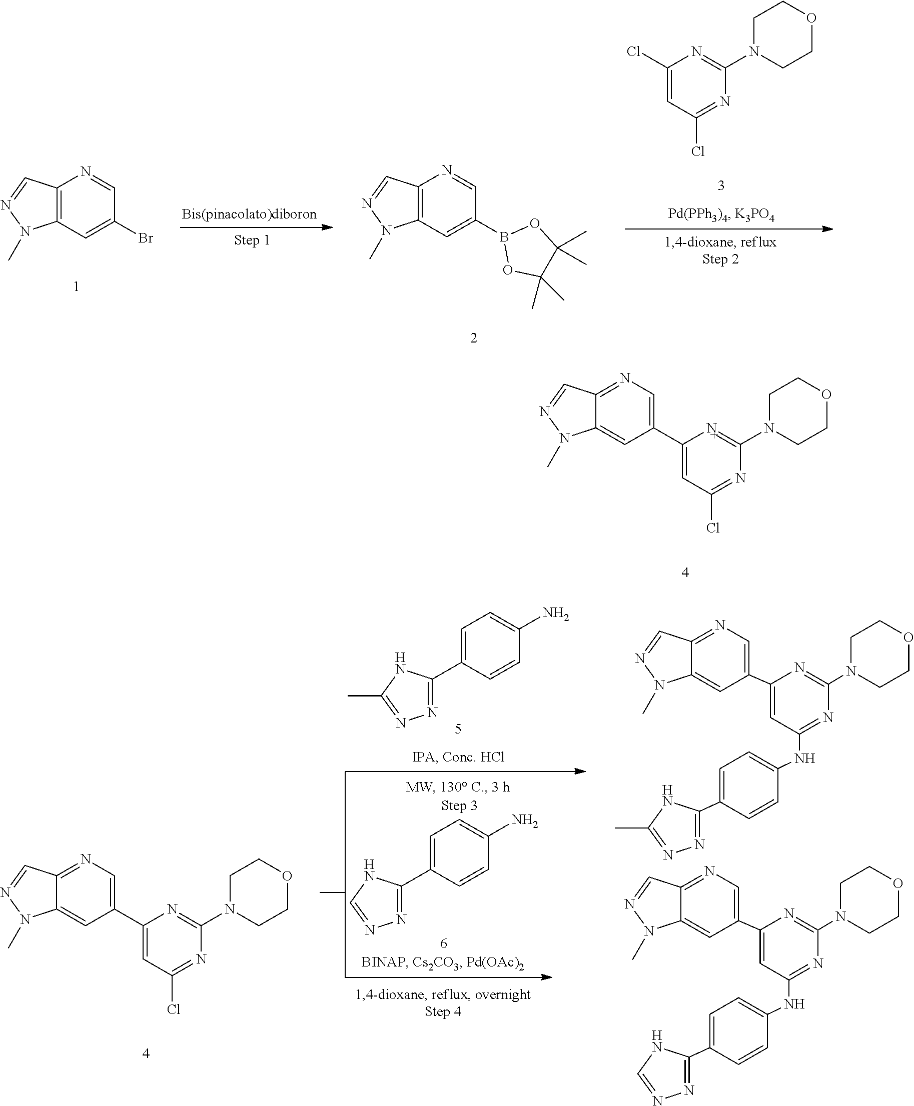

### Step 1: Synthesis of 1-methyl-6-(4,4,5,5-tetramethyl-1,3,2-dioxaborolan-2-yl)-1H-pyrazolo[4,3-b] pyridine (2)

The title compound has been synthesized by following the general procedure described above for Boronate ester preparation by using bromo compound 1 and Bis (pinacolato)diboron. LCMS (m/z): 178.00 (M +1; boronic acid).

### Step 2: Synthesis of 4-(4-chloro-6-(1-methyl-1H-pyrazolo[4,3-b]pyridin-6-yl)pyrimidin-2-yl)morpholine (4)

The title compound has been synthesized by following the general procedure described above for Suzuki coupling by using chloro compound 3 and Boronate ester 2. LCMS (m/z): 331.05 (M+1).

### Step 3: Synthesis of 6-(1-methyl-1H-pyrazolo[4,3-b]pyridin-6-yl)-N-(4-(5-methyl-4H-1,2,4-triazol-3-yl) phenyl)-2-morpholinopyrimidin-4-amine

The title compound has been synthesized by following the general procedure described above for displacement reaction (IPA/HCl) by using chloro compound 4 and amino compound 5. HPLC purity: 99.58%; ¹H NMR (400 MHz, DMSO-d₆) δ: 9.65 (s, 1H), 9.12 (d, J=1.8 Hz, 1H), 8.67 (s, 1H), 8.33 (s, 1H), 7.96-7.89 (m, 2H), 7.76 (d, J=8.3 Hz, 2H), 6.79 (s, 1H), 4.18 (d, J=1.4 Hz, 3H), 3.83 (d, J=4.9 Hz, 4H), 3.78-3.70 (m, 4H), 2.34 (s, 3H); LCMS Calculated for C₂₄H₂₄N₁₀O: 468.21; Observed: 469.35 (M+1).

### Step 4: Synthesis of N-(4-(4H-1,2,4-triazol-3-yl)phenyl)-6-(1-methyl-1H-pyrazolo[4,3-b]pyridin-6-yl)-2-morpholinopyrimidin-4-amine

The title compound has been synthesized by following the general procedure described above for Buchwald coupling by using chloro compound 4 and amino compound 6. HPLC purity: 91.57%; ¹H NMR (400 MHz, DMSO-d₆) δ: 9.74 (s, 1H), 9.12 (d, J=1.8 Hz, 1H), 8.67 (s, 1H), 8.34 (s, 1H), 7.99 (d, J=8.3 Hz, 2H), 7.82 (d, J=8.4 Hz, 2H), 6.80 (s, 1H), 4.18 (s, 3H), 3.82-3.70 (m, 8H); LCMS Calculated for C₂₃H₂₂N₁₀: 454.20; Observed: 455.30 (M+1).

### Example 176

### Synthesis of N-(4-(4H-1,2,4-triazol-3-yl)phenyl)-6-(1,3-dimethyl-1H-pyrazolo[4,3-b]pyridin-6-yl)-2-morpholinopyrimidin-4-amine

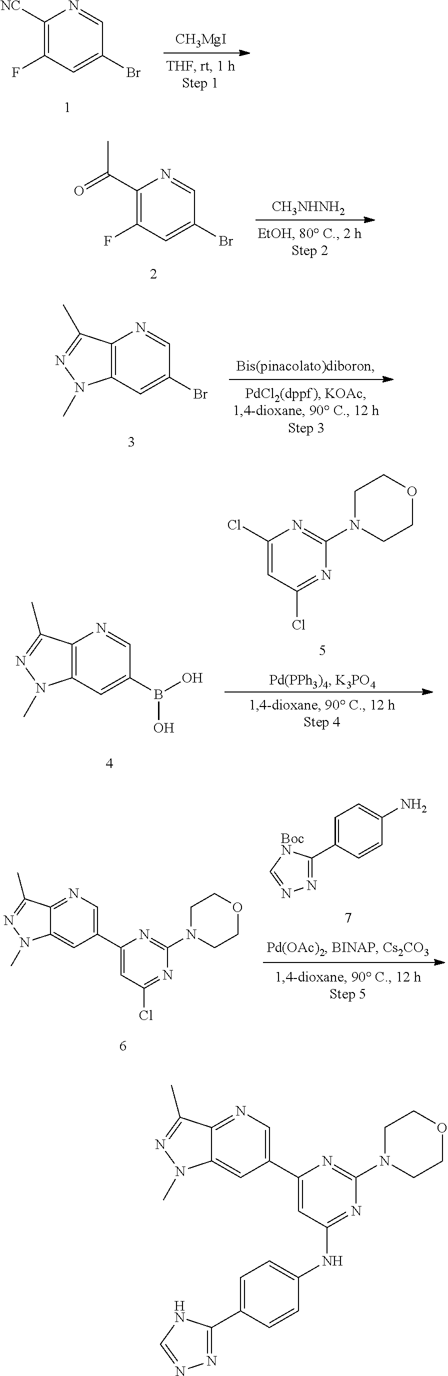

### Step 1: Synthesis of 1-(5-bromo-3-fluoropyridin-2-yl)ethanone (2)

To a stirred solution of compound 1 (4 g, 1 eq) in dry THF (10 mL), methyl magnesium iodide (9.91 g, 3 eq) was added at 0° C. and the resulting reaction mixture was stirred at room temperature for 1 h. The progress of the reaction was monitored by TLC. After completion of the reaction, the reaction mixture was quenched with saturated NH₄Cl solution. The residue was diluted with water and extracted with ethyl acetate (3×50 mL). Combined organic extracts were dried over anhydrous sodium sulfate and evaporated under reduced pressure. The crude product was purified by column chromatography on silica gel 100-200 mesh using 20% EtOAc-hexane to afford the title compound 2. LCMS (m/z): 217.85 (M+1).

### Step 2: Synthesis of 6-bromo-1,3-dimethyl-1H-pyrazolo[4,3-b]pyridine (3)

To a stirred solution of compound 2 (1.5 g, 1 eq) in ethanol (20 mL), methyl hydrazine (2.5 g, 8 eq) was added and the resulting reaction mixture was stirred at 80° C. for 2 h. The progress of the reaction was monitored by TLC. After completion of the reaction, the reaction mixture was evaporated to dryness. The residue was diluted with water and extracted with ethyl acetate (3×50 mL). Combined organic extracts were dried over anhydrous sodium sulfate and evaporated under reduced pressure. The crude product was purified by column chromatography on silica gel 100-200 mesh using 10% EtOAc-hexane to afford the title compound 3. LCMS (m/z): 227.85 (M+1).

### Step 3: Synthesis of (1,3-dimethyl-1H-pyrazolo[4,3-b]pyridin-6-yl)boronic acid (4)

The title compound (crude product) has been synthesized by following the general procedure described above for Boronate ester preparation by using bromo compound 3 and Bis (pinacolato)diboron. LCMS (m/z): 191.00 (M+1).

### Step 4: Synthesis of 4-(4-chloro-6-(1,3-dimethyl-1H-pyrazolo[4,3-b]pyridin-6-yl)pyrimidin-2-yl) morpholine (6)

The title compound has been synthesized by following the general procedure described above for Suzuki coupling by using chloro compound 5 and Boronic acid 4. LCMS (m/z): 345.00 (M+1).

### Step 5: Synthesis of N-(4-(4H-1,2,4-triazol-3-yl)phenyl)-6-(1,3-dimethyl-1H-pyrazolo[4,3-b]pyridin-6-yl) -2-morpholinopyrimidin-4-amine

The title compound has been synthesized by following the general procedure described above for Buchwald coupling by using chloro compound 6 and amino compound 7. HPLC purity: 99.29%; ¹H NMR (400 MHz, DMSO-d₆) δ: 14.07 (s, 1H), 9.71 (s, 1H), 9.05 (s, 1H), 8.59 (s, 1H), 7.98 (d, J=8.3 Hz, 2H), 7.82 (d, J=8.4 Hz, 2H), 6.78 (s, 1H), 4.09 (s, 3H), 3.89-3.77 (m, 8H), 2.56 (s, 3H). LCMS Calculated for C₂₄H₂₄N₁₀O: 468.21; Observed: 469.40 (M+1).

### Example 177

### Synthesis of N-(4-chlorophenyl)-6-(1,3-dimethyl-1H-pyrazolo[4,3-c]pyridin-6-yl)-2-morpholinopyrimidin-4-amine

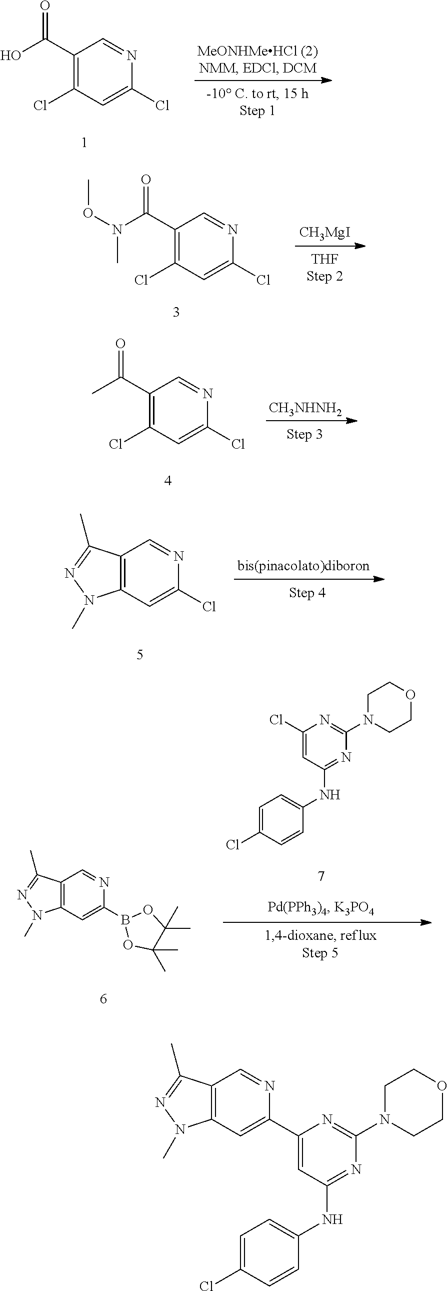

### Step 1: Synthesis of 4,6-dichloro-N-methoxy-N-methylnicotinamide (3)

To a stirred solution of compound 1 (1.2 g, 1 eq) in dichloromethane (15 mL), N-methyl morpholine (1.36 mL, 2 eq), N, O-dimethyl hydroxyl amine hydrochloride (0.735 g, 1.2 eq) and EDCI.HCl (1.4 g, 1.2 eq) were added at −10° C. and stirred at room temperature for 15 h. The progress of the reaction was monitored by TLC. After completion of the reaction, the reaction mixture was diluted with water and extracted with ethyl acetate (3×50 mL). Combined organic extracts were washed with brine and dried over anhydrous sodium sulfate and evaporated under reduced pressure. The crude product was purified by column chromatography on silica gel 100-200 mesh using 50% EtOAc-hexane to afford the title compound 3. LCMS (m/z): 234.95 (M+1).

### Step 2: Synthesis of 1-(4,6-dichloropyridin-3-yl)ethan-1-one (4)

To a stirred solution of compound 3 (1 g, 1 eq) in THF (10 mL),methyl magnesium iodide (2.8 mL, 2 eq) was added at 0° C. and stirred at room temperature for 15 h. The progress of the reaction was monitored by TLC. After completion of the reaction, the reaction mixture was quenched with saturated ammonium chloride solution and extracted with ethyl acetate (3×25 mL). Combined organic extracts were washed with brine and dried over anhydrous sodium sulfate and evaporated under reduced pressure. The crude product was purified by column chromatography on silica gel 100-200 mesh using 30% EtOAc-hexane to afford the title compound 4. ¹H NMR (400 MHz, CDCl₃) δ: 8.61 (s, 1H), 7.45 (s, 1H), 2.68 (s, 3H).

### Step 3: Synthesis of 6-chloro-1,3-dimethyl-1H-pyrazolo[4,3-c]pyridine (5)

To a stirred solution of compound 4 (0.3 g, 1 eq)in ethanol (5 mL), methyl hydrazine(0.16 mL, 2 eq)was added and heated at 50° C. for 2 h. The progress of the reaction was monitored by TLC. After completion of the reaction, the reaction mixture was evaporated under reduced pressure. The residue was dissolved in water and extracted with ethyl acetate (2×25 mL). Combined organic extracts were washed with brine, dried over anhydrous sodium sulfate and evaporated under reduced pressure. The crude product was purified by column chromatography on silica gel 100-200 mesh using 20% EtOAc-hexane to afford the title compound 5. LCMS (m/z): 182.00 (M+1).

### Step 4: Synthesis of 1,3-dimethyl-6-(4,4,5,5-tetramethyl-1,3,2-dioxaborolan-2-yl)-1H-pyrazolo[4,3-c]pyridine (6)

The title compound (crude product) has been synthesized by following the general procedure described above for Boronate ester preparation by using bromo compound 5 and Bis (pinacolato) diboron.

### Step 5: Synthesis of N-(4-chlorophenyl)-6-(1,3-dimethyl-1H-pyrazolo[4,3-c]pyridin-6-yl)-2-morpholinopyrimidin-4-amine

The title compound has been synthesized by following the general procedure described above for Suzuki coupling by using chloro compound 7 and Boronate ester 6. ¹H NMR (400 MHz, CDC13) δ: 8.98 (s, 1H), 8.32 (s, 1H), 7.48-7.41 (m, 2H), 7.33 (d, J=8.4 Hz, 2H), 7.22 (s, 1H), 6.59 (s, 1H), 4.08 (s, 3H), 3.88-3.78 (m, 8H), 2.65 (s, 3H); HPLC purity: 99.02%; LCMS Calculated for C₂₂H₂₂ClN₇O:435.16; Observed: 436.30 (M+1).

### Example 178

### Synthesis of N-(4-chlorophenyl)-6-(1-methyl-1H-pyrazolo[3,4-b]pyridin-6-yl)-2-morpholinopyrimidin-4-amine

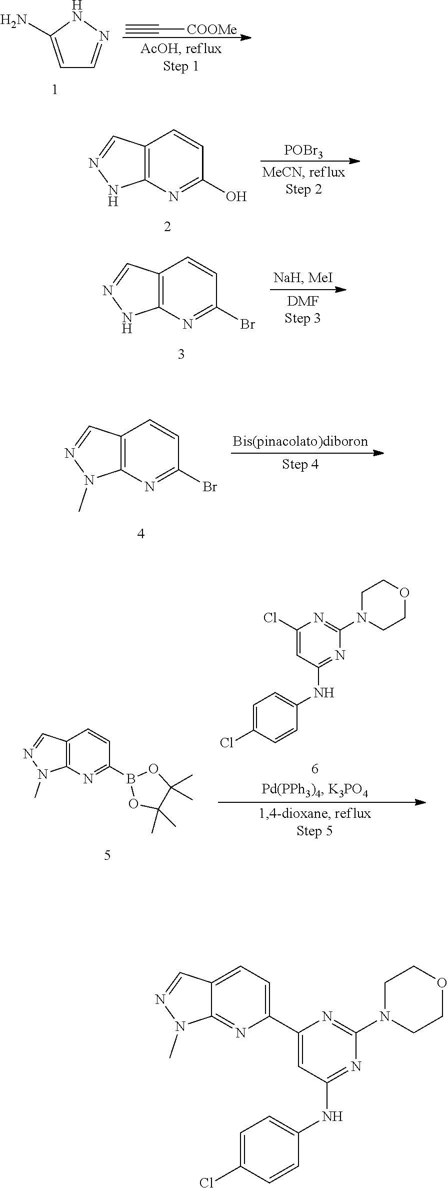

### Step 1: Synthesis of 1H-pyrazolo[3,4-b]pyridin-6-ol (2)

To a stirred solution of 1H-pyrazol-5-amine 1(10 g, 1 eq)in acetic acid (130 mL), methyl propiolate (11.09 g, 1.1 eq) was added and heated to reflux for 4 h. The progress of the reaction was monitored by TLC. After completion of the reaction, the reaction mixture was evaporated under reduced pressure. To the oily residue ethanol, ethyl acetate and pentane were added and the obtained solid was filtered and dried to afford the title compound 2. LCMS (m/z): 136.05 (M+1).

### Step 2: Synthesis of 6-bromo-1H-pyrazolo[3,4-b]pyridine (3)

To a stirred solution of compound 2 (6 g, 1 eq) in acetonitrile (60 mL), POBr₃ (18.87 g, 1.5 eq) was added and heated to reflux for 6 h. The progress of the reaction was monitored by TLC. After completion of the reaction, the reaction mixture was evaporated to dryness. The residue was basified with saturated sodium bicarbonate solution and extracted with ethyl acetate (3×50 mL). Combined organic extracts were dried over anhydrous sodium sulfate and evaporated under reduced pressure. The crude product was purified by column chromatography on silica gel 100-200 mesh using 10% MeOH-DCM to afford the title compound 3.

### Step 3: Synthesis of 6-bromo-1-methyl-1H-pyrazolo[3,4-b]pyridine (4)

To a stirred solution of compound 3 (6 g,1 eq) in DMF (60 mL), NaH (1.45 g, 1.2 eq) was added portion wise followed by the addition of methyl iodide (5.16 g, 1.2 eq) and stirred at room temperature for 15 min. The progress of the reaction was monitored by TLC. After completion of the reaction, the reaction mixture was quenched with ice water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 10% EtOAc-hexane to afford the title compound 4. LCMS (m/z): 213.95 (M+2).

### Step 4: Synthesis of 1-methyl-6-(4,4,5,5-tetramethyl-1,3,2-dioxaborolan-2-yl)-1H-pyrazolo[3,4-b]pyridine (5)

The title compound (crude product) has been synthesized by following the general procedure described above for Boronate ester preparation by using bromo compound 4 and Bis (pinacolato) diboron.

### Step 5: Synthesis of N-(4-chlorophenyl)-6-(1-methyl-1H-pyrazolo[3,4-b]pyridin-6-yl)-2-morpholino pyrimidin-4-amine

The title compound has been synthesized by following the general procedure described above for Suzuki coupling by using compound 6 and Boronate ester 5. ¹H NMR (400 MHz, DMSO-d₆) δ: 9.86 (s, 1H), 8.38 (d, J=8.3 Hz, 1H), 8.28-8.19 (m, 2H), 7.80-7.71 (m, 2H), 7.43-7.34 (m, 2H), 7.29 (s, 1H), 4.14 (s, 3H), 3.82-3.74 (m, 8H); HPLC purity: 96.95%; LCMS Calculated for C₂₁H₂₀ClN₇O: 421.14; Observed: 422.25 (M+1).

### Example 179

### Synthesis of N-(4-chlorophenyl)-6-(1,3-dimethyl-1H-pyrazolo[3,4-b]pyridin-6-yl)-2-morpholinopyrimidin-4-amine

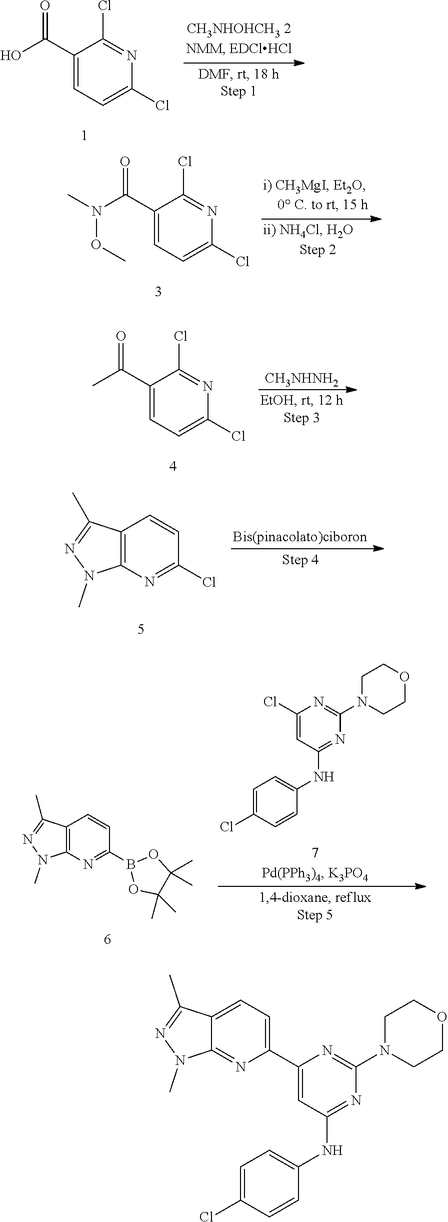

### Step 1: Synthesis of 2,6-dichloro-N-methoxy-N-methylnicotinamide (3)

To a stirred solution of compound 1 (4 g, 1 eq) in dichloromethane (50 mL), N-methyl morpholine (4.22 g, 1.2 eq), N, O-dimethyl hydroxyl amine hydrochloride (2.45 g, 1.2 eq) and EDCI.HCl (4.82 g, 1.2 eq) were added and stirred at room temperature for 18 h. The progress of the reaction was monitored by TLC. After completion of the reaction, the reaction mixture was diluted with water and extracted with ethyl acetate (3×50 mL). Combined organic extracts were washed with brine and dried over anhydrous sodium sulfate and evaporated under reduced pressure. The crude product was purified by column chromatography on silica gel 100-200 mesh using 50% EtOAc-hexane to afford the title compound 3. ¹H NMR (400 MHz, DMSO-d₆) δ: 8.13-8.08 (m, 1H), 7.76-7.68 (m, 1H), 3.47 (s, 3H), 3.30 (s, 3H).

### Step 2: Synthesis of 1-(4,6-dichloropyridin-3-yl)ethan-1-one (4)

To a stirred solution of compound 3 (3 g, 1 eq) in THF (40 mL),methyl magnesium iodide (12.76 mL, 3 eq) was added at 0° C. and stirred at room temperature for 15 h. The progress of the reaction was monitored by TLC. After completion of the reaction, the reaction mixture was quenched with saturated ammonium chloride solution and extracted with ethyl acetate (3×50 mL). Combined organic extracts were washed with brine and dried over anhydrous sodium sulfate and evaporated under reduced pressure. The crude product was purified by column chromatography on silica gel 100-200 mesh using 30% EtOAc-hexane to afford the title compound 4.

### Step 3: Synthesis of 6-chloro-1,3-dimethyl-1H-pyrazolo[3,4-b]pyridine (5)

To a stirred solution of compound 4 (0.8 g, 1 eq)in ethanol (10 mL), methyl hydrazine(0.54 g, 2 eq)was added and stirred at room temperature for 15 h. The progress of the reaction was monitored by TLC. After completion of the reaction, the reaction mixture was evaporated under reduced pressure. The residue was dissolved in water and extracted with ethyl acetate (3×25 mL). Combined organic extracts were washed with brine, dried over anhydrous sodium sulfate and evaporated under reduced pressure. The crude product was purified by column chromatography on silica gel 100-200 mesh using 20% EtOAc-hexane to afford the title compound 5. LCMS (m/z): 181.90 (M+1).

### Step 4: Synthesis of 1,3-dimethyl-6-(4,4,5,5-tetramethyl-1,3,2-dioxaborolan-2-yl)-1H-pyrazolo[3,4-b]pyridine (6):

The title compound has been synthesized by following the general procedure described above for Boronate ester preparation by using bromo compound 5 and Bis (pinacolato) diboron.

### Step 5: Synthesis of N-(4-chlorophenyl)-6-(1,3-dimethyl-1H-pyrazolo[3,4-b]pyridin-6-yl)-2-morpholino pyrimidin-4-amine

The title compound has been synthesized by following the general procedure described above for Suzuki coupling by using chloro compound 7 and Boronate ester 6. ¹H NMR (400 MHz, DMSO-d₆) δ: 9.93 (s, 1H), 8.34 (d, J=8.3 Hz, 1H), 8.17 (d, J=8.3 Hz, 1H), 7.75 (d, J=8.4 Hz, 2H), 7.38 (d, J=8.4 Hz, 2H), 7.28 (s, 1H), 4.06 (s, 3H), 3.78-3.68 (m, 8H), 2.53 (s, 3H); HPLC purity: 98.85%; LCMS Calculated for C₂₂H₂₂ClN₇O:435.16; Observed: 436.30 (M+1).

### Examples 180-183

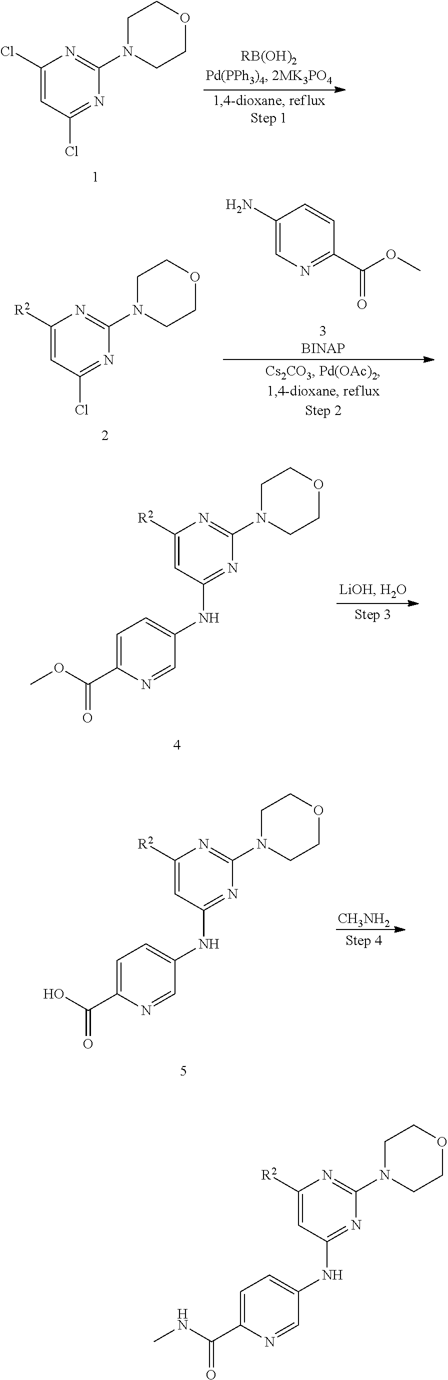

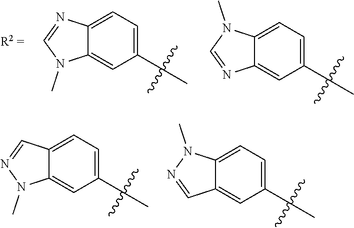 [SMILES: *c1ccc2ncn(C)c2c1 ; *c1ccc2c(c1)ncn2C ; *c1ccc2cnn(C)c2c1 ; *c1ccc2c(cnn2C)c1]

### Step 1: General procedure for Suzuki Coupling

To a mixture of dichloro compound 1 (1 eq), boronic acid/boronate ester (1 eq) in 1,4-dioxane, 2M solution of potassium phosphate was added and purged with argon for 15 min followed by the addition of tetrakistriphenyl phosphine palladium (0.06 eq) and stirred at 90° C. for overnight. The progress of the reaction was monitored by TLC. After completion of the reaction, the reaction mixture was filtered through celite and evaporated to dryness. The residue was taken in ethyl acetate, washed with water, brine, dried over anhydrous sodium sulfate and evaporated under reduced pressure. The crude product was purified by column chromatography on silica gel to afford the desired product 2.

The following intermediates were prepared in a similar manner starting with appropriate boronic acid/boronate ester.

| Structure | LCMS |
|---|---|
| 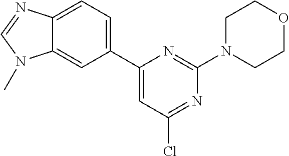 [SMILES: Cn1cnc2ccc(-c3cc(Cl)nc(N4CCOCC4)n3)cc21] | LCMS (m/z): 330.25 (M + 1) |
| 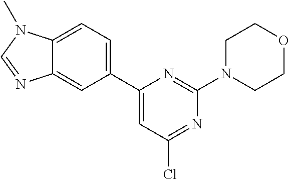 [SMILES: Cn1cnc2cc(-c3cc(Cl)nc(N4CCOCC4)n3)ccc21] | LCMS (m/z): 330.15 (M + 1) |
| 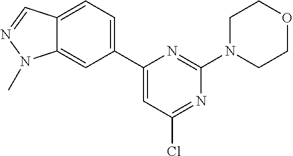 [SMILES: Cn1ncc2ccc(-c3cc(Cl)nc(N4CCOCC4)n3)cc21] | LCMS (m/z): 330.15 (M + 1) |
| 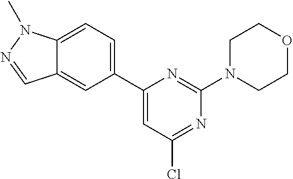 [SMILES: Cn1ncc2cc(-c3cc(Cl)nc(N4CCOCC4)n3)ccc21] | LCMS (m/z): 330.30 (M + 1) |

### Step 2: General Procedure for Buchwald Coupling

A mixture of corresponding chloro compound 2(1 eq), methyl 5-aminopicolinate3, (1 eq), cesium carbonate (1.5 eq) in 1,4-dioxane was taken and purged with argon for 10 min, followed by the addition of BINAP (0.22 eq) and purged argon for additional 5 min. Palladium acetate (0.2 eq) was added and stirred at 100° C. for overnight. The progress of the reaction was monitored by TLC. After completion of the reaction, the reaction mixture was filtered through celite and evaporated to dryness. The residue was taken in ethyl acetate, washed with water, brine, dried over anhydrous sodium sulfate and evaporated under reduced pressure. The crude product was purified by column chromatography on silica gel to afford the desired product 4.

The following compounds were prepared in a similar manner starting with appropriate chloro compound 2.

| Structure | LCMS |
|---|---|
| 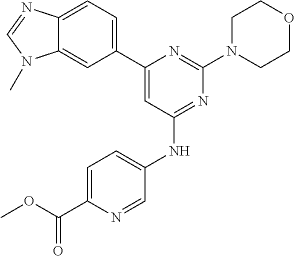 [SMILES: COC(=O)c1ccc(Nc2cc(-c3ccc4ncn(C)c4c3)nc(N3CCOCC3)n2)cn1] | LCMS (m/z): 446.40 (M + 1) |
| 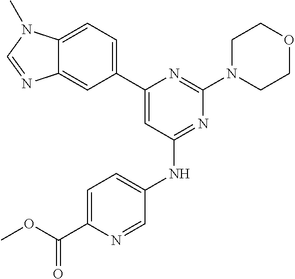 [SMILES: COC(=O)c1ccc(Nc2cc(-c3ccc4c(c3)ncn4C)nc(N3CCOCC3)n2)cn1] | LCMS (m/z): 468.05 (M + 23) |
| 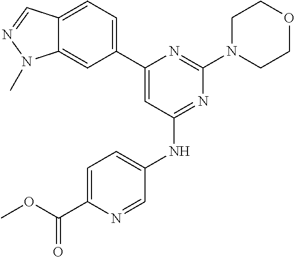 [SMILES: COC(=O)c1ccc(Nc2cc(-c3ccc4cnn(C)c4c3)nc(N3CCOCC3)n2)cn1] | LCMS (m/z): 446.15 (M + 1) |
| 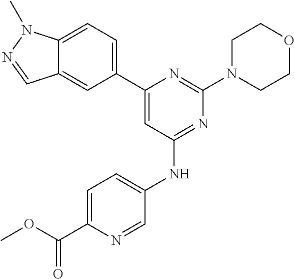 [SMILES: COC(=O)c1ccc(Nc2cc(-c3ccc4c(cnn4C)c3)nc(N3CCOCC3)n2)cn1] | LCMS (m/z): 446.30 (M + 1) |

### Step 3: General Procedure for Ester Hydrolysis

To a stirred solution of compound 4 (1 eq) in THF:H₂O (1:1), lithium hydroxide (2 eq) in minimum amount of water was added and stirred at room temperature for 2 h. The progress of the reaction was monitored by TLC. After completion of the reaction, excess of ethyl acetate was added and aqueous layer has been separated. Aqueous layer was acidified with 1N HCl, filtered off the solid obtained and dried in vacuo to afford acid 5. The crude product has been used as such for the next step without further purification.

The following compounds were prepared in a similar manner starting with appropriate ester compound.

| Structure | LCMS |
|---|---|
| 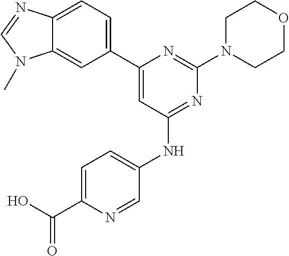 [SMILES: Cn1cnc2ccc(-c3cc(Nc4ccc(C(=O)O)nc4)nc(N4CCOCC4)n3)cc21] | LCMS (m/z): 432.85 (M + 1) |
| 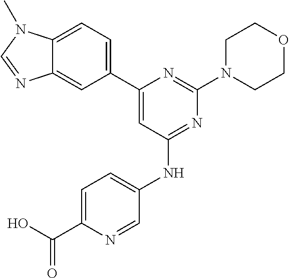 [SMILES: Cn1cnc2cc(-c3cc(Nc4ccc(C(=O)O)nc4)nc(N4CCOCC4)n3)ccc21] | LCMS (m/z): 432.20 (M + 1) |
| 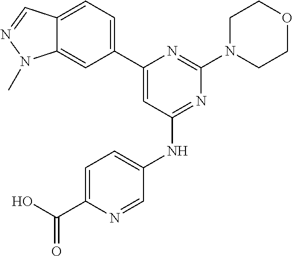 [SMILES: Cn1ncc2ccc(-c3cc(Nc4ccc(C(=O)O)nc4)nc(N4CCOCC4)n3)cc21] | LCMS (m/z): 432.30 (M + 1) |
| 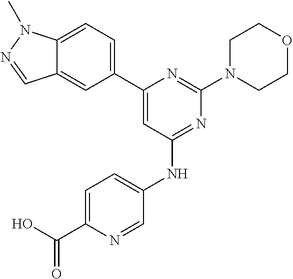 [SMILES: Cn1ncc2cc(-c3cc(Nc4ccc(C(=O)O)nc4)nc(N4CCOCC4)n3)ccc21] | — |

### Step 4: General Procedure for Amide Coupling

To a mixture of Acid 5 (1 eq), HATU (1.5 eq) in DMF, DIPEA (2.5 eq) was added and stirred at room temperature for 10 min. Methyl amine hydrochloride (1.2 eq) was added slowly and stirred at room temperature for 2 h. The progress of the reaction was monitored by TLC. After completion of the reaction, water was added and extracted with ethyl acetate. Combined organic extracts were dried over anhydrous sodium sulfate and evaporated under reduced pressure. The crude product was purified by column chromatography/preparative HPLC to afford the desired product.

N-methyl-5-((6-(1-methyl-1H-benzo[d]imidazol-6-yl)-2-morpholinopyrimidin-4-yl)amino)picolinamide: The title compound has been synthesized by following the general procedure described above for Amide coupling by using the corresponding Acid 5. ¹H NMR (MeOD, 400 MHz) δ: 9.58 (s, 1H), 9.14 (s, 1H), 8.60 (s, 1H), 8.40 (d, J=8.3 Hz, 1H), 8.23 (d, J=8.8 Hz, 1H), 8.15 (d, J=8.2 Hz, 1H), 8.07 (d, J=8.6 Hz, 1H), 6.83 (s, 1H), 4.28 (s, 3H), 3.98-3.86 (m, 8H), 2.99 (s, 3H); HPLC purity: 95.35%; LCMS Calculated for C₂₃H₂₄N₈O₂ (free base): 444.20; Observed: 445.25 (M+1).

N-methyl-5-((6-(1-methyl-1H-benzo[d]imidazol-5-yl)-2-morpholinopyrimidin-4-yl)amino)picolinamide: The title compound has been synthesized by following the general procedure described above for Amide coupling by using the corresponding Acid 5. ¹H NMR (DMSO-d₆, 400 MHz) δ: 9.44 (s, 1H), 8.93 (s, 1H), 8.46 (d, J=4.3 Hz, 1H), 8.25 (dd, J=13.0, 8.6 Hz, 2H), 8.01 (t, J=8.9 Hz, 2H), 6.75 (s, 1H), 4.06 (s, 3H), 3.83-3.71 (m, 8H), 2.80 (s, 3H); HPLC purity: 95.82%; LCMS Calculated for C₂₃H₂₄N₈O₂ (free base): 444.20; Observed: 445.20(M+1).

N-methyl-5-((6-(1-methyl-1H-indazol-6-yl)-2-morpholinopyrimidin-4-yl)amino)picolinamide: The title compound has been synthesized by following the general procedure described above for Amide coupling by using the corresponding Acid 5. ¹H NMR (DMSO-d₆, 400 MHz) δ: 10.41 (s, 1H), 9.07-9.02 (m, 1H), 8.67 (q, J=5.2, 4.7 Hz, 1H), 8.32-8.23 (m, 2H), 8.15-8.01 (m, 2H), 7.89 (d, J=8.2 Hz, 1H), 7.73 (d, J=8.6 Hz, 1H), 6.84 (s, 1H), 4.14 (s, 3H), 3.88-3.75 (m, 8H), 2.82 (d, J=4.9 Hz, 3H); HPLC purity: 96.23%; LCMS Calculated for C₂₃H₂₄N₈O₂ (free base): 444.20; Observed: 445.30 (M+1).

N-methyl-5-((6-(1-methyl-1H-indazol-5-yl)-2-morpholinopyrimidin-4-yl)amino)picolinamide: The title compound has been synthesized by following the general procedure described above for Amide coupling by using the corresponding Acid 5. ¹H NMR (DMSO-d₆, 400 MHz) δ: 10.47 (s, 1H), 9.03 (s, 1H), 8.67 (d, J=6.3 Hz, 1H), 8.45 (s, 1H), 8.32-8.19 (m, 2H), 8.05 (d, J=8.4 Hz, 1H), 7.98 (d, J=8.6 Hz, 1H), 7.77 (d, J=8.7 Hz, 1H), 6.79 (s, 1H), 4.09 (s, 3H), 3.86-3.80 (m, 4H), 3.78-3.72 (m, 4H), 2.81 (d, J=4.4 Hz, 3H); HPLC purity: 97.25%; LCMS Calculated for C₂₃H₂₄N₈O₂ (free base): 444.20; Observed: 445.30 (M+1).

### Examples 184-185

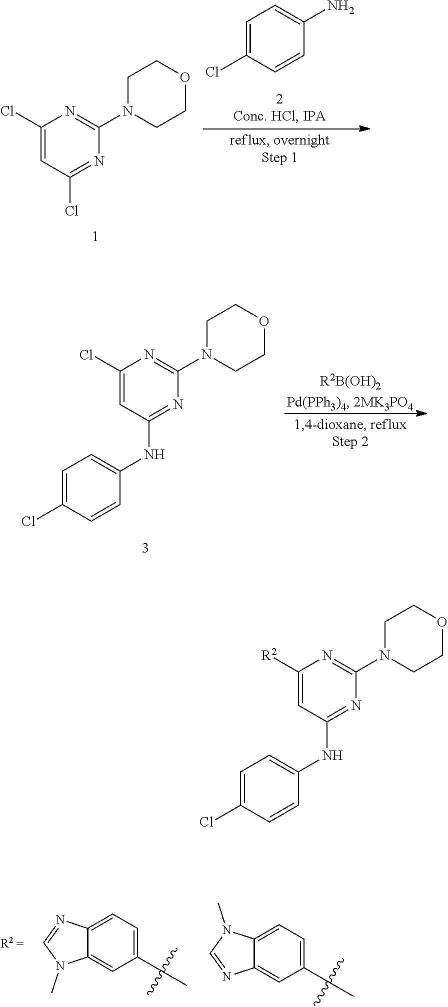

### Step 1: Synthesis of 6-chloro-N-(4-chlorophenyl)-2-morpholinopyrimidin-4-amine (3)

To a stirred solution of 4-(4,6-dichloropyrimidin-2-yl)morpholino 1(1 g, 1 eq) and 4-chloroaniline 2 (0.547 g, 1 eq) in isopropanol (10 mL), concentrated HCl (2 mL) was added and heated to reflux at 100° C. for overnight. The progress of the reaction was monitored by TLC. After completion of the reaction, the reaction mixture was evaporated to dryness. The residue was taken in ethyl acetate (50 mL), washed with 1 N HCl, brine, dried over anhydrous sodium sulfate and evaporated under reduced pressure. The crude product was purified by column chromatography on silica gel 100-200 mesh using 20% EtOAc-hexane to afford the title compound 3. LCMS (m/z):325.20(M+1).

### Step 2

N-(4-chlorophenyl)-6-(1-methyl-1H-benzo[d]imidazol-6-yl)-2-morpholinopyrimidin-4-amine: The title compound has been synthesized by following the general procedure described above for Suzuki coupling by using chloro compound 3 and corresponding boronic acid. ¹H NMR (400 MHz, DMSO-d₆) δ: 9.94 (s, 1H), 9.62 (s, 1H), 8.53 (s, 1H), 8.18 (dd, J=8.8, 1.6 Hz, 1H), 7.98 (d, J=8.7 Hz, 1H), 7.78-7.69 (m, 2H), 7.43-7.34 (m, 2H), 6.77 (s, 1H), 4.14 (s, 3H), 3.82-3.70 (m, 8H); HPLC purity: 98.73%; LCMS Calculated for C₂₂H₂₁ClN₆O (free base): 420.15; Observed: 421.20 (M+1).

N-(4-chlorophenyl)-6-(1-methyl-1H-benzo[d]imidazol-5-yl)-2-morpholinopyrimidin-4-amine: The title compound has been synthesized by following the general procedure described above for Suzuki coupling by using chloro compound 3 and corresponding boronic acid. ¹H NMR (400 MHz, DMSO-d₆) δ: 10.14 (s, 1H), 9.63 (s, 1H), 8.42 (s, 1H), 8.19 (d, J=8.8 Hz, 1H), 8.08 (d, J=8.8 Hz, 1H), 7.79-7.70 (m, 2H), 7.44-7.35 (m, 2H), 6.80 (s, 1H), 4.10 (s, 3H), 3.84-3.69 (m, 8H); HPLC purity: 96.31%; LCMS Calculated for C₂₂H₂₁ClN₆O (free base): 420.15; Observed: 421.15 (M+1).

### Examples 186-189

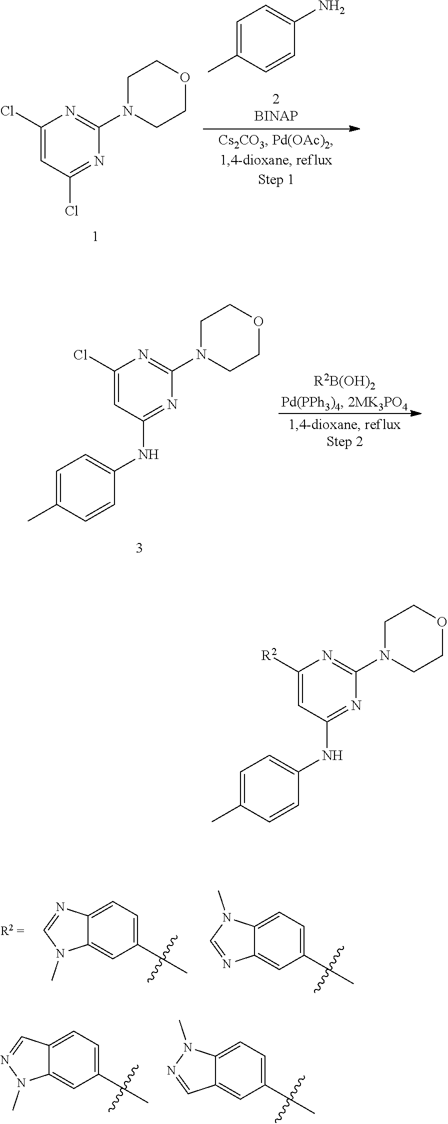

### Step 1: Synthesis of 6-chloro-2-morpholino-N-(p-tolyl)pyrimidin-4-amine (3)

The title compound has been synthesized by following the general procedure described above for Buchwald Coupling by using the chloro compound 1 and 4-methyl aniline 2. LCMS (m/z): 305.15 (M+1).

### Step 2

6-(1-methyl-1H-benzo[d]imidazol-6-yl)-2-morpholino-N-(p-tolyl)pyrimidin-4-amine: The title compound has been synthesized by following the general procedure described above for Suzuki coupling by using chloro compound 3 and corresponding boronic acid. ¹H NMR (400 MHz, CD₃OD) δ: 9.57 (s, 1H), 8.51 (s, 1H), 8.06 (s, 2H), 7.52 (d, J=7.8 Hz, 2H), 7.27 (d, J=7.9 Hz, 2H), 6.61 (s, 1H), 4.26 (s, 3H), 3.95-3.82 (m, 8H), 2.37 (s, 3H); HPLC purity: 99.17%; LCMS Calculated for C₂₃H₂₄N₆O (free base): 400.20; Observed: 401.15 (M+1).

6-(1-methyl-1H-benzo[d]imidazol-5-yl)-2-morpholino-N-(p-tolyl)pyrimidin-4-amine: The title compound has been synthesized by following the general procedure described above for Suzuki coupling by using chloro compound 3 and corresponding boronic acid. ¹H NMR (400 MHz, CD₃OD) δ: 9.56 (s, 1H), 8.34 (s, 1H), 8.16 (d, J=8.6 Hz, 1H), 8.06 (d, J=8.7 Hz, 1H), 7.54-7.47 (m, 2H), 7.26 (d, J=8.0 Hz, 2H), 6.58 (s, 1H), 4.22 (s, 3H), 3.93-3.80 (m, 8H), 2.37 (s, 3H); HPLC purity: 95.91%; LCMS Calculated for C₂₃H₂₄N₆O (free base): 400.20; Observed: 401.20 (M+1).

6-(1-methyl-1H-indazol-6-yl)-2-morpholino-N-(p-tolyl)pyrimidin-4-amine: The title compound has been synthesized by following the general procedure described above for Suzuki coupling by using chloro compound 3 and corresponding Boronate ester. ¹H NMR (400 MHz, DMSO-d₆) δ: 9.31 (s, 1H), 8.21 (s, 1H), 8.09 (s, 1H), 7.84 (d, J=8.5 Hz, 1H), 7.75 (d, J=8.5 Hz, 1H), 7.55 (d, J=8.2 Hz, 2H), 7.13 (d, J=8.1 Hz, 2H), 6.67 (s, 1H), 4.12 (s, 3H), 3.84-3.70 (m, 8H), 2.27 (s, 3H); HPLC purity: 99.49%; LCMS Calculated for C₂₃H₂₄N₆O: 400.16; Observed: 401.25 (M+1).

6-(1-methyl-1H-indazol-5-yl)-2-morpholino-N-(p-tolyl)pyrimidin-4-amine: The title compound has been synthesized by following the general procedure described above for Suzuki coupling by using chloro compound 3 and corresponding boronic acid. ¹H NMR (400 MHz, DMSO-d₆) δ: 8.35 (s, 1H), 8.24 (s, 1H), 7.82 (t, J=8.4 Hz, 2H), 7.56 (d, J=7.9 Hz, 2H), 7.20 (d, J=8.0 Hz, 2H), 6.66 (s, 1H), 4.10 (s, 3H), 3.85-3.70 (m, 8H), 2.29 (s, 3H); HPLC purity: 97.24%; LCMS Calculated for C₂₃H₂₄N₆O (free base): 400.20; Observed: 401.20 (M+1).

### Example 190

### Synthesis of 6-(1H-benzo [d]imidazol-6-yl)-N-(4-chlorophenyl)-2-morpholinopyrimidin-4-amine

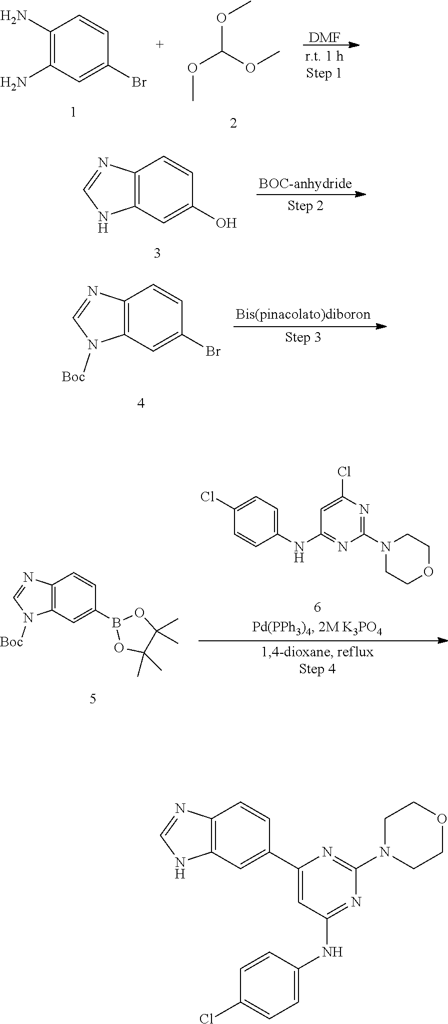

### Step 1: Synthesis of 6-bromo-1H-benzo[d]imidazole (3)

To a stirred solution of 4-bromobenzene-1,2-diamine 1 (5 g,1 eq) and trimethylorthoformate (73 mL) in DMF (36 mL), conc. HCl (2.5 mL) was added drop wise and the reaction mixture was stirred at room temperature for 1 h. The progress of the reaction was monitored by TLC. After completion of the reaction, the reaction mixture was diluted with water and extracted with ethyl acetate (3×100 mL). Combined organic layers were dried over anhydrous sodium sulfate and concentrated under reduced pressure. The crude product was purified by column chromatography on silica gel 100-200 mesh using 60% EtOAc-hexane to afford the title compound 3. LCMS (m/z): 198.90(M+1).

### Step 2: Synthesis of tert-butyl 6-bromo-1H-benzo[d]imidazole-1-carboxylate (4)

To a stirred solution of 6-bromo-1H-benzo[d]imidazole 3(1 g, 1 eq) in THF (20 mL), triethylamine (0.9 mL,1.3 eq) was added and stirred for 15 min followed by the addition of Boc anhydride(1.5 mL,1.3 eq).The reaction mixture was stirred at room temperature for 4 h. The progress of the reaction was monitored by TLC. After completion of the reaction, the reaction mixture was concentrated under reduced pressure. The residue was diluted with water and extracted with ethyl acetate (2×50 mL). Combined organic extracts were dried over anhydrous sodium sulfate and evaporated under reduced pressure. The crude product was purified by column chromatography on silica gel 100-200 mesh using 30% EtOAc-hexane to afford the title compound 4. LCMS (m/z): 196.85(M-Boc).

### Step 3: Synthesis tert-butyl 6-(4,4,5,5-tetramethyl-1,3,2-dioxaborolan-2-yl)-1H-benzo[d]imidazole-1-carboxylate (5)

The title compound (crude product) has been synthesized by following the general procedure described above for Boronate ester preparation by using bromo compound 4 and Bis (pinacolato)diboron. LCMS (m/z): 245.10 (M-Boc).

### Step 4: Synthesis of 6-(1H-benzo[d]imidazol-6-yl)-N-(4-chlorophenyl)-2-morpholinopyrimidin-4-amine

The title compound has been synthesized by following the general procedure described above for Suzuki coupling by using compound 6 and Boronate ester 5. ¹H NMR (400 MHz, DMSO-d₆) δ: 10.08 (s, 1H), 9.64 (s, 1H), 8.41 (s, 1H), 8.16 (d, J=8.6 Hz, 1H), 7.97 (d, J=8.6 Hz, 1H), 7.75 (d, J=8.6 Hz, 2H), 7.43-7.34 (m, 2H), 6.78 (s, 1H), 3.80-3.72(m, 8H); HPLC purity: 97.40%; LCMS Calculated for C₂₁H₁₉ClN₆O: 406.13; Observed: 407 (M+1).

### Example 191

### Synthesis of N-(4-chlorophenyl)-6-(1-cyclopropyl-1H-benzo [d]imidazol-6-yl)-2-morpholinopyrimidin-4-amine

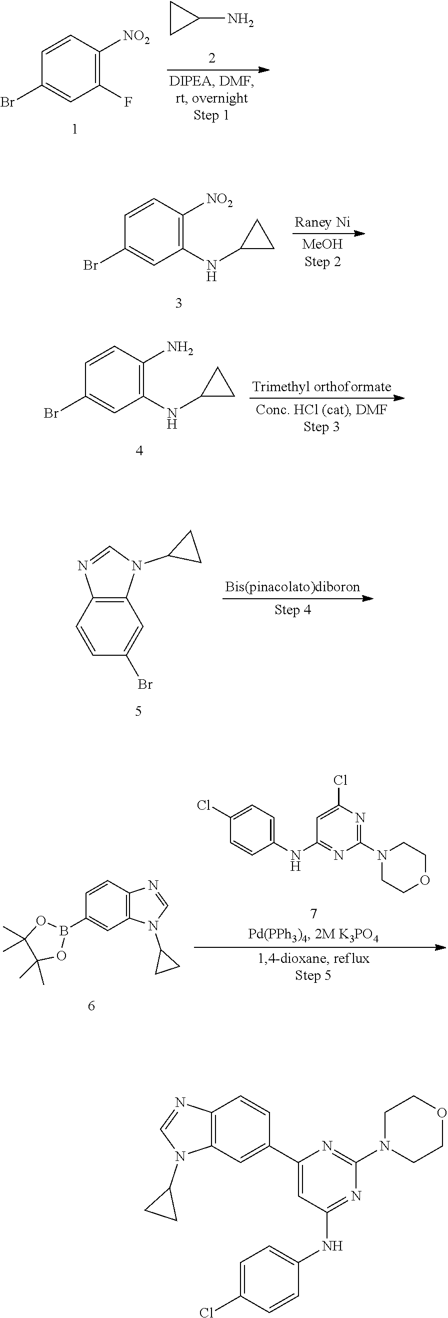

### Step 1: Synthesis of 5-bromo-N-cyclopropyl-2-nitroaniline (3)

To a stirred solution of 4-bromo-2-fluoro-1-nitrobenzene1(2 g,1 eq), cyclopropylamine (0.518 g, 1 eq)in DMF (10 mL), DIPEA (2.34 g, 2 eq) was added and stirred at room temperature for overnight. The progress of the reaction was monitored by TLC. After completion of the reaction, the reaction mixture was quenched with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 2% EtOAc-hexane to afford the title compound 3. LCMS (m/z):257.00 (M+1).

### Step 2: Synthesis of 5-bromo-N1-cyclopropylbenzene-1,2-diamine (4)

To a stirred solution of 5-bromo-N-cyclopropyl-2-nitroaniline3(2.3 g,1 eq), in methanol (20 mL), Raney nickel (2.6 g) was added and stirred at room temperature for 18 h under hydrogen atmosphere. The progress of the reaction was monitored by TLC. After completion of the reaction, the reaction mixture was concentrated under reduced pressure to afford the title compound 4. 96%; LCMS (m/z):227.15 (M+1).

### Step 3: Synthesis of 6-bromo-1-cyclopropyl-1H-benzo[d]imidazole (5)

To a stirred solution of 5-bromo-N1-cyclopropylbenzene-1,2-diamine 4 (0.3 g,1 eq), in DMF (5 mL), trimethylorthoformate (4.4 mL) and Conc. HCl (0.15 mL)were added and stirred at room temperature for 1 h. The progress of the reaction was monitored by TLC. After completion of the reaction, the reaction mixture was diluted with water and extracted with ethyl acetate (2×25 mL). Combined organic extracts were dried over anhydrous sodium sulfate and evaporated under reduced pressure. The crude product was purified by column chromatography on silica gel 100-200 mesh using 50% EtOAc-hexane to afford the title compound 5. LCMS (m/z):238.90 (M+1).

### Step 4: Synthesis of 1-cyclopropyl-6-(4,4,5,5-tetramethyl-1,3,2-dioxaborolan-2-yl)-1H-benzo[d]imidazole (6)

The title compound (crude product) has been synthesized by following the general procedure described above for Boronate ester preparation by using bromo compound 5 and Bis (pinacolato)diboron. LCMS (m/z):285.10 (M+1).

### Step 5: Synthesis of N-(4-chlorophenyl)-6-(1-cyclopropyl-1H-benzo[d]imidazol-6-yl)-2-morpholinopyrimidin-4-amine

The title compound has been synthesized by following the general procedure described above for Suzuki coupling by using compound 7 and Boronate ester 6. ¹H NMR (400 MHz, DMSO-d₆) δ: 9.98 (s, 1H), 9.66 (s, 1H), 8.46 (s, 1H), 8.19 (d, J=8.6 Hz, 1H), 7.96 (d, J=8.6 Hz, 1H), 7.78-7.70 (m, 2H), 7.44-7.35 (m, 2H), 6.78 (s, 1H), 3.92 (tt, J=7.4, 4.3 Hz, 1H), 3.80-3.72 (m, 8H), 1.31-1.19 (m, 4H); HPLC purity: 96.83%; LCMS Calculated for C₂₄H₂₃ClN₆O: 446.16; Observed: 447.20 (M+1).

### Examples 192-199

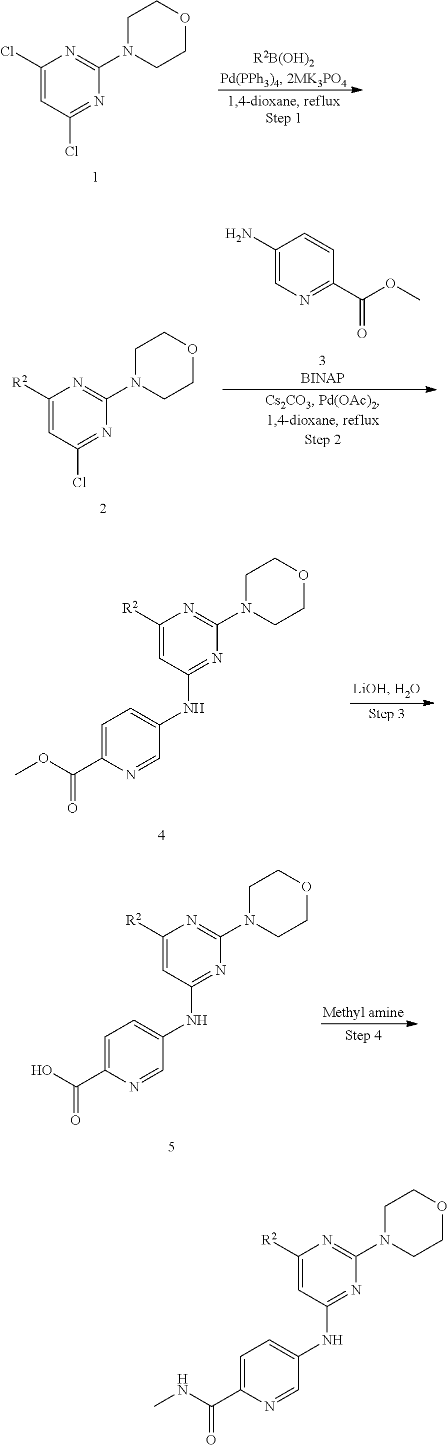

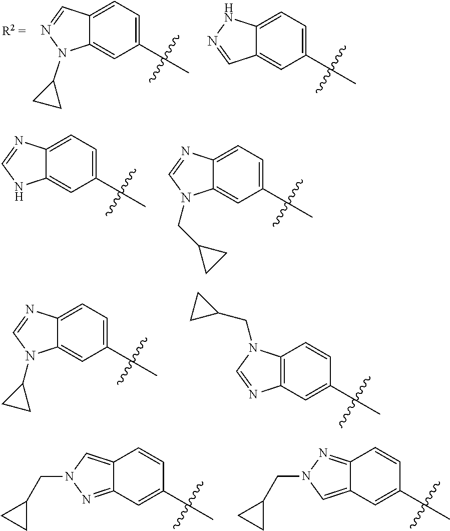

### Step 1: General procedure for Suzuki Coupling

To a mixture of dichloro compound 1 (1 eq), boronic acid/boronate ester (1 eq) in 1,4-dioxane, 2M solution of potassium phosphate was added and purged with argon for 15 min followed by the addition of tetrakistriphenyl phosphine palladium (0.06 eq) and stirred at 90° C. for overnight. The progress of the reaction was monitored by TLC. After completion of the reaction, the reaction mixture was filtered through celite and evaporated to dryness. The residue was taken in ethyl acetate, washed with water, brine, dried over anhydrous sodium sulfate and evaporated under reduced pressure. The crude product was purified by column chromatography on silica gel to afford the desired product 2.

The following intermediates were prepared in a similar manner starting with appropriate boronic acid/boronate ester.

| Structure | LCMS |
|---|---|
| 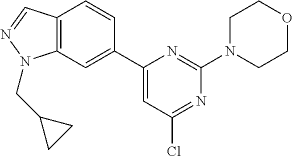 [SMILES: Clc1cc(-c2ccc3cnn(CC4CC4)c3c2)nc(N2CCOCC2)n1] | LCMS (m/z): 370.10 (M + 1) |
| 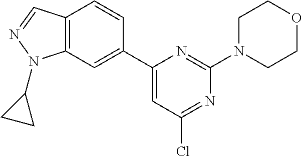 [SMILES: Clc1cc(-c2ccc3cnn(C4CC4)c3c2)nc(N2CCOCC2)n1] | LCMS (m/z): 356.05 (M + 1) |
| 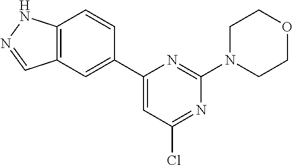 [SMILES: Clc1cc(-c2ccc3[nH]ncc3c2)nc(N2CCOCC2)n1] | LCMS (m/z): 316.00 (M + 1) |
| 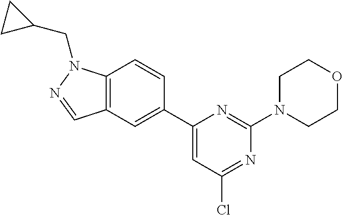 [SMILES: Clc1cc(-c2ccc3c(cnn3CC3CC3)c2)nc(N2CCOCC2)n1] | LCMS (m/z): 370.10 (M + 1) |
| 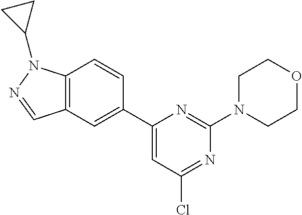 [SMILES: Clc1cc(-c2ccc3c(cnn3C3CC3)c2)nc(N2CCOCC2)n1] | LCMS (m/z): 356.10 (M + 1) |
| 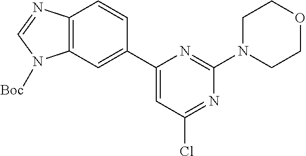 [SMILES: CC(C)(C)OC(=O)n1cnc2ccc(-c3cc(Cl)nc(N4CCOCC4)n3)cc21] | LCMS (m/z): 416.15 (M + 1). |
| 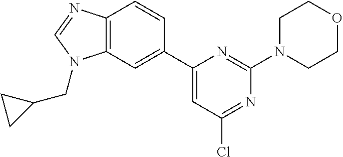 [SMILES: Clc1cc(-c2ccc3ncn(CC4CC4)c3c2)nc(N2CCOCC2)n1] | LCMS (m/z): 370.10 (M + 1). |
| 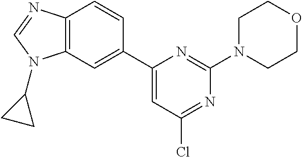 [SMILES: Clc1cc(-c2ccc3ncn(C4CC4)c3c2)nc(N2CCOCC2)n1] | LCMS (m/z): 356.10 (M + 1). |
| 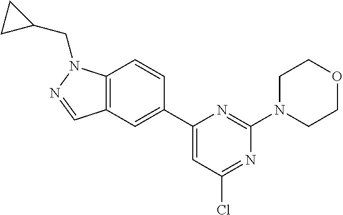 [SMILES: Clc1cc(-c2ccc3c(cnn3CC3CC3)c2)nc(N2CCOCC2)n1] | LCMS (m/z): 370.15 (M + 1). |
| 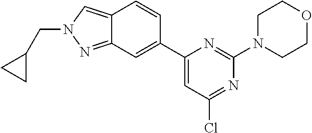 [SMILES: Clc1cc(-c2ccc3cn(CC4CC4)nc3c2)nc(N2CCOCC2)n1] | LCMS (m/z): 370.20 (M + 1). |
| 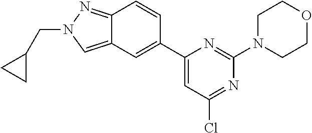 [SMILES: Clc1cc(-c2ccc3nn(CC4CC4)cc3c2)nc(N2CCOCC2)n1] | LCMS (m/z): 370.10 (M + 1). |

### Step 2: General Procedure for Buchwald Coupling

A mixture of corresponding chloro compound 2 (1 eq), methyl 5-aminopicolinate3 (1 eq), cesium carbonate (1.5 eq) in 1,4-dioxane was taken and purged with argon for 10 min, followed by the addition of BINAP (0.22 eq) and purged with argon for additional 5 min. Palladium acetate (0.2 eq) was added and stirred at 100° C. for overnight. The progress of the reaction was monitored by TLC. After completion of the reaction, the reaction mixture was filtered through celite and evaporated to dryness. The residue was taken in ethyl acetate, washed with water, brine, dried over anhydrous sodium sulfate and evaporated under reduced pressure. The crude product was purified by column chromatography on silica gel to afford the desired product 4.

The following compounds were prepared in a similar manner starting with appropriate chloro compound 2.

| Structure | LCMS |
|---|---|
| 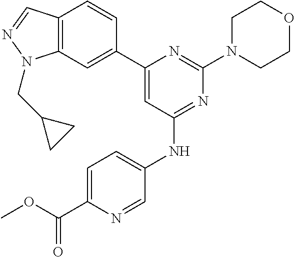 [SMILES: COC(=O)c1ccc(Nc2cc(-c3ccc4cnn(CC5CC5)c4c3)nc(N3CCOCC3)n2)cn1] | LCMS (m/z): 486.25 (M + 1) |
| 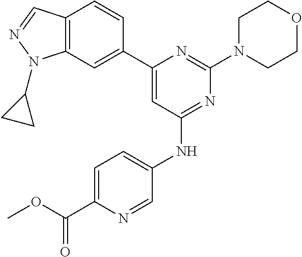 [SMILES: COC(=O)c1ccc(Nc2cc(-c3ccc4cnn(C5CC5)c4c3)nc(N3CCOCC3)n2)cn1] | LCMS (m/z): 472.25 (M + 23) |
| 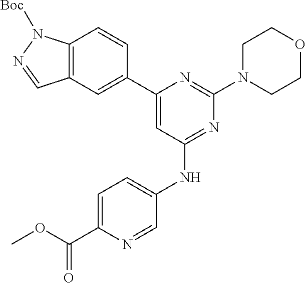 [SMILES: COC(=O)c1ccc(Nc2cc(-c3ccc4c(cnn4C(=O)OC(C)(C)C)c3)nc(N3CCOCC3)n2)cn1] | LCMS (m/z): 532.40 (M + 1) |
| 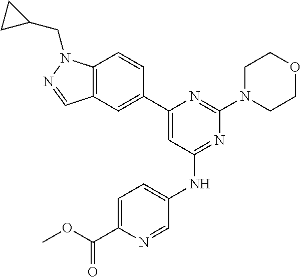 [SMILES: COC(=O)c1ccc(Nc2cc(-c3ccc4c(cnn4CC4CC4)c3)nc(N3CCOCC3)n2)cn1] | LCMS (m/z): 486.30 (M + 1) |
| 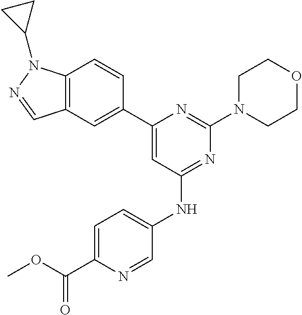 | LCMS (m/z): 458.25 (M + 1) |
| 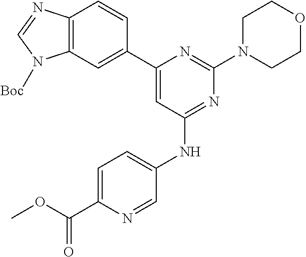 [SMILES: COC(=O)c1ccc(Nc2cc(-c3ccc4ncn(C(=O)OC(C)(C)C)c4c3)nc(N3CCOCC3)n2)cn1] | LCMS (m/z): 532.25 (M + 1) |
| 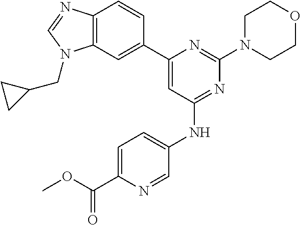 [SMILES: COC(=O)c1ccc(Nc2cc(-c3ccc4ncn(CC5CC5)c4c3)nc(N3CCOCC3)n2)cn1] | LCMS (m/z): 485.22 (M+) |
| 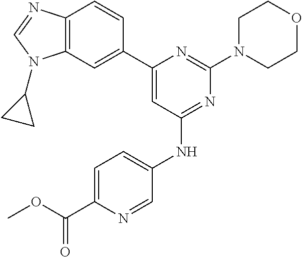 [SMILES: COC(=O)c1ccc(Nc2cc(-c3ccc4ncn(C5CC5)c4c3)nc(N3CCOCC3)n2)cn1] | LCMS (m/z): 472 (M + 1). |
| 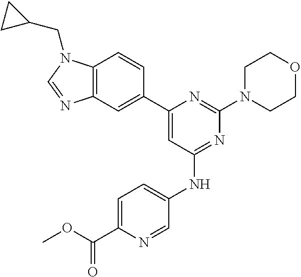 [SMILES: COC(=O)c1ccc(Nc2cc(-c3ccc4c(c3)ncn4CC3CC3)nc(N3CCOCC3)n2)cn1] | LCMS (m/z): 486.30 (M + 1). |
| 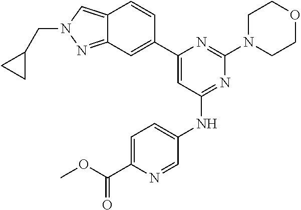 [SMILES: COC(=O)c1ccc(Nc2cc(-c3ccc4cn(CC5CC5)nc4c3)nc(N3CCOCC3)n2)cn1] | LCMS (m/z): 486.40 (M + 1). |
| 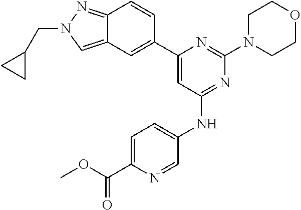 [SMILES: COC(=O)c1ccc(Nc2cc(-c3ccc4nn(CC5CC5)cc4c3)nc(N3CCOCC3)n2)cn1] | LCMS (m/z): 486.30 (M + 1). |

### Step 3: General Procedure for Ester Hydrolysis

To a stirred solution of compound 4 (1 eq) in THF:H₂O (1:1), lithium hydroxide (2 eq) in minimum amount of water was added and stirred at room temperature for 2 h. The progress of the reaction was monitored by TLC. After completion of the reaction, excess of ethyl acetate was added and aqueous layer has been separated. Aqueous layer was acidified with 1N HCl, filtered off the solid obtained and dried in vacuo to afford acid 5. The crude product has been used as such for the next step without further purification.

The following compounds were prepared in a similar manner starting with appropriate ester compound.

| Structure | LCMS |
|---|---|
| 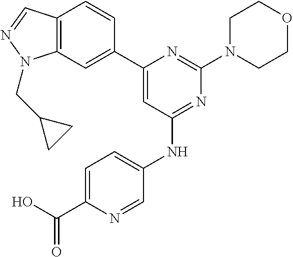 [SMILES: O=C(O)c1ccc(Nc2cc(-c3ccc4cnn(CC5CC5)c4c3)nc(N3CCOCC3)n2)cn1] | LCMS (m/z): 472.15 (M + 1) |
| 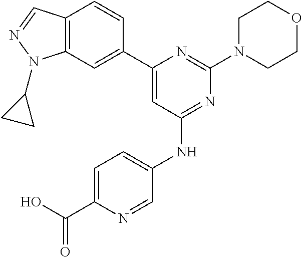 [SMILES: O=C(O)c1ccc(Nc2cc(-c3ccc4cnn(C5CC5)c4c3)nc(N3CCOCC3)n2)cn1] | LCMS (m/z): 458.25 (M + 1) |
| 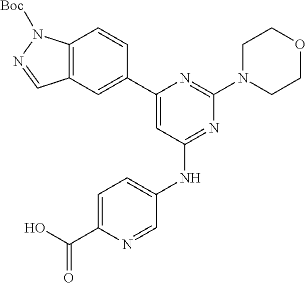 [SMILES: CC(C)(C)OC(=O)n1ncc2cc(-c3cc(Nc4ccc(C(=O)O)nc4)nc(N4CCOCC4)n3)ccc21] | LCMS (m/z): 518.30 (M + 1) |
| 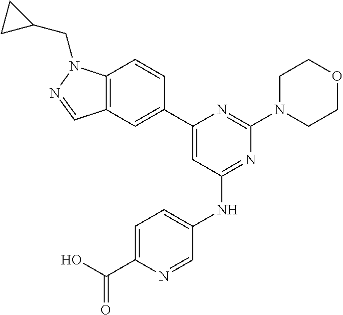 [SMILES: O=C(O)c1ccc(Nc2cc(-c3ccc4c(cnn4CC4CC4)c3)nc(N3CCOCC3)n2)cn1] | LCMS (m/z): 472.20 (M + 1) |
| 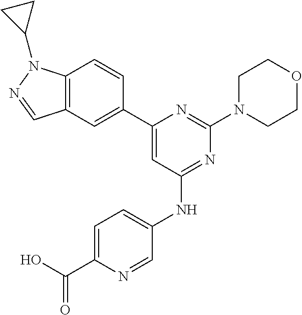 [SMILES: O=C(O)c1ccc(Nc2cc(-c3ccc4c(cnn4C4CC4)c3)nc(N3CCOCC3)n2)cn1] | LCMS (m/z): 458.25 (M + 1) |
| 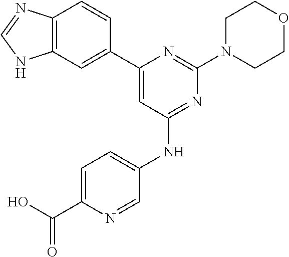 [SMILES: O=C(O)c1ccc(Nc2cc(-c3ccc4nc[nH]c4c3)nc(N3CCOCC3)n2)cn1] | LCMS (m/z): 417.85 (M + 1) |
| 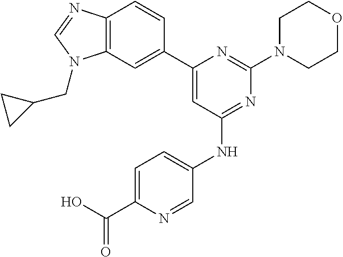 [SMILES: O=C(O)c1ccc(Nc2cc(-c3ccc4ncn(CC5CC5)c4c3)nc(N3CCOCC3)n2)cn1] | LCMS (m/z): 471.25 (M + 1) |
| 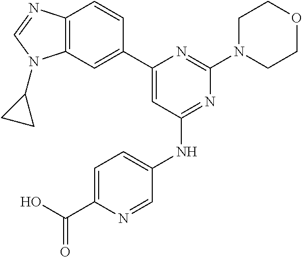 [SMILES: O=C(O)c1ccc(Nc2cc(-c3ccc4ncn(C5CC5)c4c3)nc(N3CCOCC3)n2)cn1] | — |
| 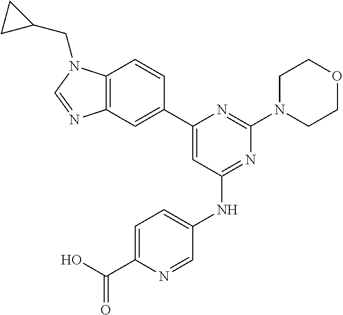 [SMILES: O=C(O)c1ccc(Nc2cc(-c3ccc4c(c3)ncn4CC3CC3)nc(N3CCOCC3)n2)cn1] | — |
| 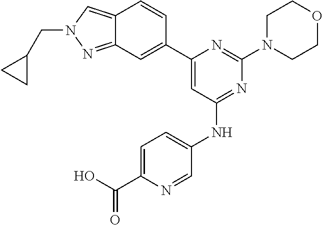 [SMILES: O=C(O)c1ccc(Nc2cc(-c3ccc4cn(CC5CC5)nc4c3)nc(N3CCOCC3)n2)cn1] | LCMS (m/z): 472.20 (M + 1). |
| 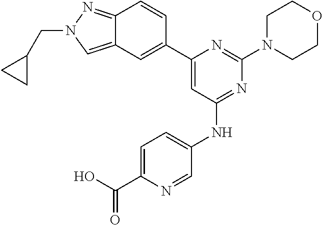 [SMILES: O=C(O)c1ccc(Nc2cc(-c3ccc4nn(CC5CC5)cc4c3)nc(N3CCOCC3)n2)cn1] | LCMS (m/z): 472.25 (M + 1). |

### Step 4: General procedure for Amide Coupling

To a mixture of Acid 5(1 eq), HATU (1.5 eq) in DMF, DIPEA (2.5 eq) was added and stirred at room temperature for 10 min. Methyl amine hydrochloride (1.2 eq) was added slowly and stirred at room temperature for 2 h. The progress of the reaction was monitored by TLC. After completion of the reaction, water was added and extracted with ethyl acetate. Combined organic extracts were dried over anhydrous sodium sulfate and evaporated under reduced pressure. The crude product was purified by column chromatography/preparative HPLC to afford the desired product.

5-46-(1-cyclopropyl-1H-indazol-6-yl)-2-morphohnopyrimidin-4-yl)amino)-N-methylpicanamide: The title compound has been synthesized by following the general procedure described above for Amide coupling by using the corresponding Acid5and methyl amine hydrochloride. ¹H NMR (400 MHz, DMSO-d₆) δ: 10.28 (s, 1H), 9.03 (d, J=2.5 Hz, 1H), 8.66 (q, J=4.6 Hz, 1H), 8.28 (d, J=7.1 Hz, 2H), 8.11-8.01 (m, 2H), 7.88 (d, J=8.5 Hz, 1H), 7.80 (d, J=8.5 Hz, 1H), 6.83 (s, 1H), 3.89-3.72 (m, 9H), 2.81 (d, J=4.6 Hz, 3H), 1.20-1.15 (m, 4H); HPLC purity: 96.58%; LCMS Calculated for C₂₅H₂₆N₈O₂: 470.22; Observed: 471.25 (M+1).

5-((6-(1H-indazol-5-yl)-2-morphohnopyrimidin-4-yl)amino)-N-methylpicanamide: The title compound has been synthesized by following the general procedure described above for Amide coupling by using the corresponding Acid 5 and methyl amine hydrochloride. ¹H NMR (400 MHz, DMSO-d₆) δ: 10.50 (s, 1H), 9.03 (d, J=2.5 Hz, 1H), 8.69 (q, J=4.9 Hz, 1H), 8.45 (s, 1H), 8.32-8.22 (m, 2H), 8.06 (d, J=8.6 Hz, 1H), 7.94 (d, J=9.0 Hz, 1H), 7.68 (d, J=8.8 Hz, 1H), 6.78 (s, 1H), 3.84-3.74 (m, 8H), 2.81 (d, J=4.6 Hz, 3H); HPLC purity: 93.46%; LCMS Calculated for C₂₂H₂₂N₈O₂: 430.19; Observed: 431.25 (M+1).

5-((6-(1H-benzo[d]imidazol-6-yl)-2-morphohnopyrimidin-4-yl)amino)-N-methylpicanamide: The title compound has been synthesized by following the general procedure described above for Amide coupling by using the corresponding Acid5and methyl amine hydrochloride. ¹H NMR (400 MHz, Methanol-d4) δ: 8.98 (d, J=2.5 Hz, 1H), 8.35 (s, 1H), 8.32-8.23 (m, 2H), 8.01 (t, J=8.6 Hz, 2H), 7.68 (d, J=8.6 Hz, 1H), 6.68 (s, 1H), 3.95-3.82 (m, 8H), 2.96 (s, 3H); HPLC purity: 99.17%; LCMS Calculated for C₂₂H₂₂N₈O₂: 430.19; Observed: 431.25 (M+1).

5-((6-(1-(cyclopropylmethyl)-1H-benzo[d]imidazol-6-yl)-2-morphohnopyrimidin-4-yl)amino)-N-methylpicanamide: The title compound has been synthesized by following the general procedure described above for Amide coupling by using the corresponding Acid5and methyl amine hydrochloride. ¹H NMR (400 MHz, DMSO-d₆) δ: 9.91 (s, 1H), 8.99 (d, J=2.5 Hz, 1H), 8.60 (q, J=4.7 Hz, 1H), 8.38 (s, 1H), 8.31-8.20 (m, 2H), 8.00 (d, J=8.6 Hz, 1H), 7.92 (dd, J=8.7, 1.6 Hz, 1H), 7.75 (d, J=8.5 Hz, 1H), 6.76 (s, 1H), 4.21 (d, J=7.1 Hz, 2H), 3.86-3.70 (m, 8H), 2.81 (d, J=4.7 Hz, 3H), 1.36-1.28 (m, 1H), 0.64-0.44 (m, 4H); HPLC purity: 98.07%; LCMS Calculated for C₂₆H₂₈N₈O₂: 484.23; Observed: 485.25 (M+1).

5-((6-(1-cyclopropyl-1H-benzo[d]imidazol-6-yl)-2-morphohnopyrimidin-4-yl)amino)-N-methylpicolinamide: The title compound has been synthesized by following the general procedure described above for Amide coupling by using the corresponding Acid 5 and methyl amine hydrochloride. ¹H NMR (400 MHz, DMSO-d₆) δ: 9.96 (s, 1H), 8.99 (d, J=2.5 Hz, 1H), 8.76 (s, 1H), 8.60 (q, J=4.8 Hz, 1H), 8.33 (s, 1H), 8.24 (dd, J=8.8, 2.5 Hz, 1H), 8.04 (dd, J=20.4, 8.5 Hz, 2H), 7.82 (d, J=8.6 Hz, 1H), 6.77 (s, 1H), 3.86-3.79 (m, 4H), 3.78-3.71 (m, 5H), 2.81 (d, J=4.8 Hz, 3H), 1.26-1.13 (m, 4H); HPLC purity: 95.47%; LCMS Calculated for C₂₅H₂₆N₈O₂: 470.22; Observed: 471.25 (M+1).

5-((6-(1-(cyclopropylmethyl)-1H-benzo[d]imidazol-5-yl)-2-morphohnopyrimidin-4-yl)amino)-N-methylpicanamide: The title compound has been synthesized by following the general procedure described above for Amide coupling by using the corresponding Acid5and methyl amine hydrochloride. ¹H NMR (400 MHz, DMSO-d₆) δ: 10.33 (s, 1H), 9.74 (d, J=6.5 Hz, 1H), 9.03 (t, J=2.6 Hz, 1H), 8.67-8.59 (m, 1H), 8.46 (s, 1H), 8.32-8.14 (m, 2H), 8.02 (dd, J=14.1, 8.7 Hz, 1H), 7.54 (s, 1H), 6.86 (d, J=6.6 Hz, 1H), 6.55 (s, 1H), 4.43 (dd, J=25.4, 7.3 Hz, 2H), 3.82-3.74 (m, 8H), 2.81 (d, J=4.7 Hz, 3H), 1.50-1.48 (m, 1H), 0.74-0.52 (m, 4H); HPLC purity: 95.78%; LCMS Calculated for C₂₆H₂₈N₈O₂: 484.23; Observed: 485.30 (M+1).

5-((6-(2-(cyclopropylmethyl)-2H-indazol-6-yl)-2-morphohnopyrimidin-4-yl)amino)-N-methylpicolinamide: The title compound has been synthesized by following the general procedure described above for Amide coupling by using the corresponding Acid5and methyl amine hydrochloride. ¹H NMR (400 MHz, DMSO-d₆) δ: 9.07 (d, J=2.4 Hz, 1H), 8.73 (q, J=4.8 Hz, 1H), 8.52 (s, 1H), 8.36-8.26 (m, 2H), 8.09 (d, J=8.6 Hz, 1H), 7.87 (d, J=8.7 Hz, 1H), 7.56 (d, J=8.9 Hz, 1H), 6.88 (s, 1H), 4.34 (d, J=7.3 Hz, 2H), 3.84-3.74 (m, 8H), 2.82 (d, J=4.5 Hz, 3H), 1.45-1.36 (m, 1H), 0.58 (dt, J=8.0, 2.9 Hz, 2H), 0.62-0.43 (m, 2H); HPLC purity: 96.85%; LCMS Calculated for C₂₆H₂₈N₈O₂: 484.23; Observed: 485.25 (M+1).

5-((6-(2-(cyclopropylmethyl)-2H-indazol-5-yl)-2-morphohnopyrimidin-4-yl)amino)-N-methylpicolinamide: The title compound has been synthesized by following the general procedure described above for Amide coupling by using the corresponding acid 5 and methyl amine hydrochloride. ¹H NMR (400 MHz, DMSO-d₆) δ: 10.65 (s, 1H), 9.04 (d, J=2.5 Hz, 1H), 8.70 (q, J=4.9 Hz, 1H), 8.62 (s, 1H), 8.43 (s, 1H), 8.29 (dd, J=8.7, 2.4 Hz, 1H), 8.07 (d, J=8.6 Hz, 1H), 7.77 (q, J=9.0 Hz, 2H), 6.78 (s, 1H), 4.33 (d, J=7.2 Hz, 2H), 3.86-3.72 (m, 8H), 2.81 (d, J=4.6 Hz, 3H), 1.42-1.34 (m, 1H), 0.60-0.40 (m, 4H); HPLC purity: 98.29%; LCMS Calculated for C₂₆H₂₈N₈O₂: 484.23; Observed: 485.30 (M+1).

### Examples 200-203

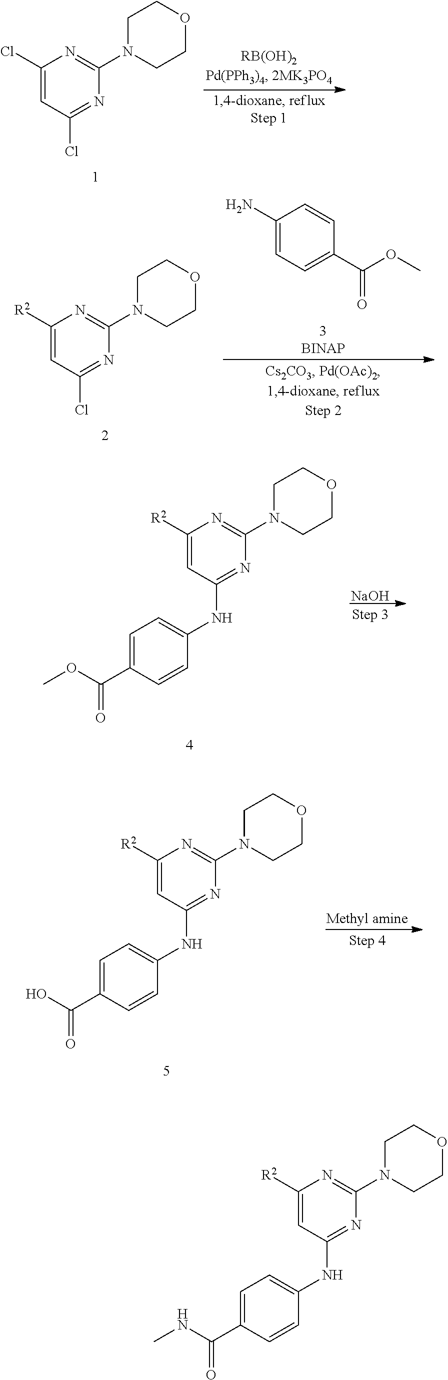

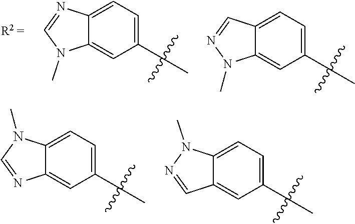 [SMILES: *c1ccc2ncn(C)c2c1 ; *c1ccc2cnn(C)c2c1 ; *c1ccc2c(c1)ncn2C ; *c1ccc2c(cnn2C)c1]

### Step 1: General Procedure for Suzuki Coupling

To a mixture of dichloro compound 1 (1 eq), boronic acid/boronate ester (1 eq) in 1,4-dioxane, 2M solution of potassium phosphate was added and purged with argon for 15 min followed by the addition of tetrakistriphenyl phosphine palladium (0.06 eq) and stirred at 90° C. for overnight. The progress of the reaction was monitored by TLC. After completion of the reaction, the reaction mixture was filtered through celite and evaporated to dryness. The residue was taken in ethyl acetate, washed with water, brine, dried over anhydrous sodium sulfate and evaporated under reduced pressure. The crude product was purified by column chromatography on silica gel to afford the desired product 2.

The following intermediates were prepared in a similar manner starting with appropriate boronic acid/boronate ester.

| Structure | LCMS |
|---|---|
| 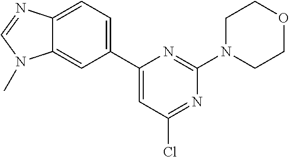 [SMILES: Cn1cnc2ccc(-c3cc(Cl)nc(N4CCOCC4)n3)cc21] | LCMS (m/z): 330.05 (M + 1) |
| 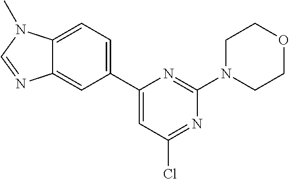 [SMILES: Cn1cnc2cc(-c3cc(Cl)nc(N4CCOCC4)n3)ccc21] | LCMS (m/z): 330.05 (M + 1) |
| 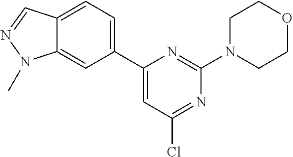 [SMILES: Cn1ncc2ccc(-c3cc(Cl)nc(N4CCOCC4)n3)cc21] | LCMS (m/z): 330.25 (M + 1) |
| 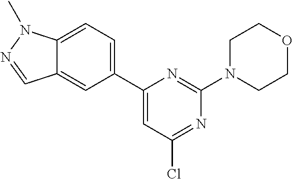 [SMILES: Cn1ncc2cc(-c3cc(Cl)nc(N4CCOCC4)n3)ccc21] | LCMS (m/z): 330.05 (M + 1) |

### Step 2: General Procedure for Buchwald Coupling

A mixture of corresponding chloro compound 2(1 eq), methyl 4-aminobenzoate 3, (1 eq), cesium carbonate (1.5 eq) in 1,4-dioxane was taken and purged with argon for 10 min, followed by the addition of BINAP (0.22 eq) and purged argon for additional 5 min. Palladium acetate (0.2 eq) was added and stirred at 100° C. for overnight. The progress of the reaction was monitored by TLC. After completion of the reaction, the reaction mixture was filtered through celite and evaporated to dryness. The residue was taken in ethyl acetate, washed with water, brine, dried over anhydrous sodium sulfate and evaporated under reduced pressure. The crude product was purified by column chromatography on silica gel to afford the desired product 4.

The following compounds were prepared in a similar manner starting with appropriate chloro compound.

| Structure | LCMS |
|---|---|
| 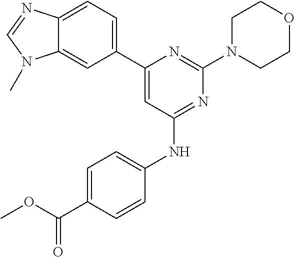 [SMILES: COC(=O)c1ccc(Nc2cc(-c3ccc4ncn(C)c4c3)nc(N3CCOCC3)n2)cc1] | LCMS (m/z): 445.20 (M + 1) |
| 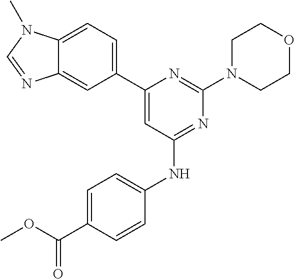 [SMILES: COC(=O)c1ccc(Nc2cc(-c3ccc4c(c3)ncn4C)nc(N3CCOCC3)n2)cc1] | LCMS (m/z): 445.30 (M + 1) |
| 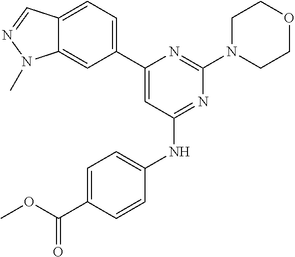 [SMILES: COC(=O)c1ccc(Nc2cc(-c3ccc4cnn(C)c4c3)nc(N3CCOCC3)n2)cc1] | LCMS (m/z): 445.30 (M + 1) |
| 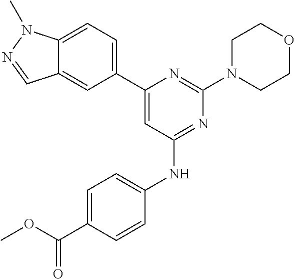 [SMILES: COC(=O)c1ccc(Nc2cc(-c3ccc4c(cnn4C)c3)nc(N3CCOCC3)n2)cc1] | LCMS (m/z): 445.30 (M + 1) |

### Step 3: General Procedure for Ester Hydrolysis

To a stirred solution of ester 3 (1 eq) in MeOH:H₂O (1:1) was added sodium hydroxide (2 eq) and refluxed for 12 h. The progress of the reaction was monitored by TLC. After completion of the reaction, the reaction mixture was evaporated under reduced pressure. The residue was dissolved in water, washed with ethyl acetate. Aqueous layer was acidified with 1N HCl and evaporated under reduced pressure to afford the crude product and used as such for the next step.

The crude following compounds were prepared in a similar manner starting with appropriate ester compound.

| Structure | LCMS |
|---|---|
| 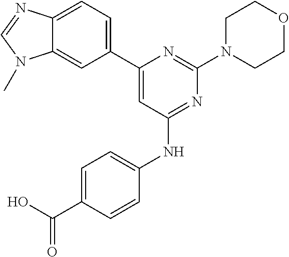 [SMILES: Cn1cnc2ccc(-c3cc(Nc4ccc(C(=O)O)cc4)nc(N4CCOCC4)n3)cc21] | LCMS (m/z): 431.25 (M + 1) |
| 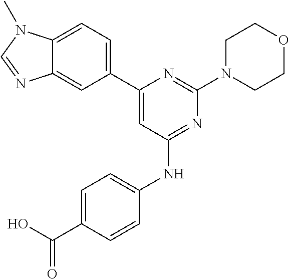 [SMILES: Cn1cnc2cc(-c3cc(Nc4ccc(C(=O)O)cc4)nc(N4CCOCC4)n3)ccc21] | LCMS (m/z): 431.20 (M + 1) |
| 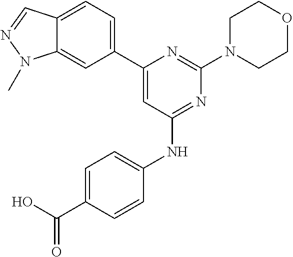 [SMILES: Cn1ncc2ccc(-c3cc(Nc4ccc(C(=O)O)cc4)nc(N4CCOCC4)n3)cc21] | LCMS (m/z): 431.20 (M + 1) |
| 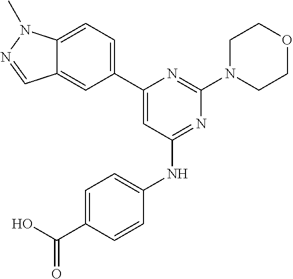 [SMILES: Cn1ncc2cc(-c3cc(Nc4ccc(C(=O)O)cc4)nc(N4CCOCC4)n3)ccc21] | LCMS (m/z): 431.35 (M + 1) |

### Step 4: General Procedure for Amide Coupling

To a mixture of Acid 5 (1 eq), PYBOP (1 eq) in DMF, DIPEA (2.5 eq) was added and stirred at room temperature for 10 min. Methyl amine hydrochloride (2 eq) was added slowly and stirred at room temperature for 2 h. The progress of the reaction was monitored by TLC. After completion of the reaction, water was added and extracted with ethyl acetate. Combined organic extracts were dried over anhydrous sodium sulfate and evaporated under reduced pressure. The crude product was purified by preparative HPLC to afford the desired product.

N-methyl-4-((6-(1-methyl-1H-benzo[d]imidazol-6-yl)-2-morpholinopyrimidin-4-yl)-amino)-benzamide: The title compound has been synthesized by following the general procedure described above for Amide coupling by using the corresponding Acid 5 and methyl amine hydrochloride. ¹H NMR (400 MHz, DMSO-d₆) δ: 9.98 (brs, 1H), 9.61 (s, 1H), 8.51 (s, 1H), 8.40-8.30 (m, 1H), 8.20 (d, J=8.6 Hz, 1H), 7.98 (d, J=8.6 Hz, 1H), 7.88-7.75 (m, 4H), 6.82 (s, 1H), 4.15 (s, 3H), 3.87-3.74 (m, 8H), 2.77 (d, J=4.2 Hz, 3H); HPLC purity: 99.42%; LCMS Calculated for C₂₄H₂₅N₇O₂: 443.21; Observed: 444.20 (M+1).

N-methyl-4-((6-(1-methyl-1H-benzo[d]imidazol-5-yl)-2-morpholinopyrimidin-4-yl)amino) benzamide: The title compound has been synthesized by following the general procedure described above for Amide coupling by using the corresponding Acid 5 and methyl amine hydrochloride. ¹H NMR (400 MHz, CD3OD) δ: 9.59 (s, 1H), 8.38 (d, J=1.4 Hz, 1H), 8.18 (d, J=8.7 Hz, 1H), 8.10 (dd, J=8.7, 1.5 Hz, 1H), 7.94-7.86 (m, 2H), 7.78 (d, J=8.3 Hz, 2H), 6.68 (s, 1H), 4.23 (s, 3H), 3.96-3.82 (m, 8H), 2.93 (s, 3H); HPLC purity: 98.44%; LCMS Calculated for C₂₄H₂₅N₇O₂: 443.21; Observed: 444.25 (M+1).

N-methyl-4-((6-(1-methyl-1H-indazol-6-yl)-2-morpholinopyrimidin-4-yl) amino)benzamide: The title compound has been synthesized by following the general procedure described above for Amide coupling by using the corresponding Acid5 and methyl amine hydrochloride. ¹H NMR (400 MHz, DMSO-d₆) δ: 10.05 (s, 1H), 8.36-8.29 (m, 1H), 8.24 (s, 1H), 8.13 (s, 1H), 7.92-7.67 (m, 6H), 6.78 (s, 1H), 4.14 (s, 3H), 3.84-3.75 (m, 8H), 2.77 (d, J=4.2 Hz, 3H); HPLC purity: 99.71%; LCMS Calculated for C₂₄H₂₅N₇O₂: 443.21; Observed: 444.25 (M+1).

N-methyl-4-((6-(1-methyl-1H-indazol-5-yl)-2-morpholinopyrimidin-4-yl)amino)benzamide: The title compound has been synthesized by following the general procedure described above for Amide coupling by using the corresponding Acid5 and methyl amine hydrochloride. ¹H NMR (400 MHz, DMSO-d₆) δ: 10.24 (s, 1H), 8.42 (s, 1H), 8.34 (d, J=4.9 Hz, 1H), 8.22 (s, 1H), 7.95 (d, J=9.0 Hz, 1H), 7.89-7.71 (m, 5H), 6.72 (s, 1H), 4.10 (s, 3H), 3.83-3.74 (m, 8H), 2.77 (d, J=4.2 Hz, 3H); HPLC purity: 95.46%; LCMS Calculated for C₂₄H₂₅N₇O₂: 443.21; Observed: 444.25 (M+1).

### Example 204

### Synthesis of N-cyclopropyl-5-((6-(1-methyl-1H-benzo[d]imidazol-6-yl)-2-morpholinopyrimidin-4-yl)amino)picolinamide

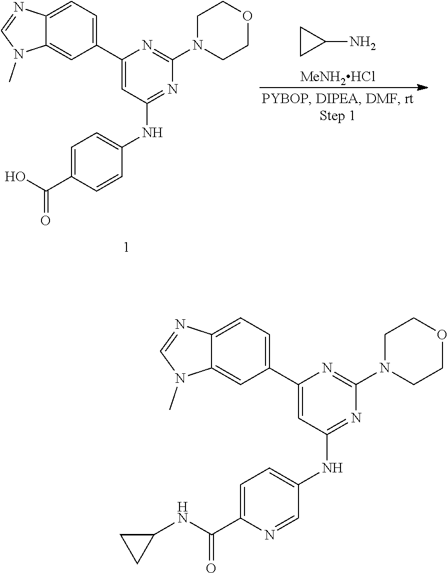

### Step 1: Synthesis of N-cyclopropyl-5-((6-(1-methyl-1H-benzo[d]imidazol-6-yl)-2-morpholinopyrimidin-4-yl)amino)picolinamide

The title compound has been synthesized by following the general procedure described above for Amide coupling by using the corresponding Acid 1 and cyclopropyl amine. ¹H NMR (400 MHz, DMSO-d₆) δ: 9.91 (s, 1H), 8.97 (d, J=2.5 Hz, 1H), 8.55 (d, J=5.0 Hz, 1H), 8.31-8.18 (m, 3H), 8.00 (d, J=8.6 Hz, 1H), 7.91 (dd, J=8.6, 1.7 Hz, 1H), 7.74 (d, J=8.5 Hz, 1H), 6.75 (s, 1H), 3.92 (s, 3H), 3.82-3.74 (m, 8H), 2.90-2.85 (m, 1H), 0.74-0.63 (m, 4H); HPLC purity: 98.41%; LCMS Calculated for C₂₅H₂₆N₈O₂: 470.22; Observed: 471.35 (M+1).

### Examples 205-236

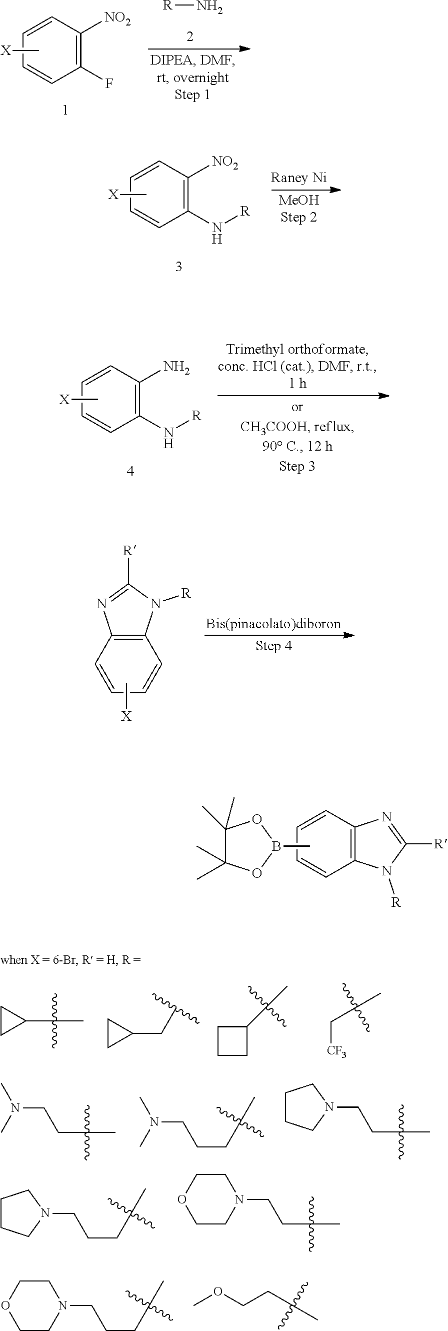

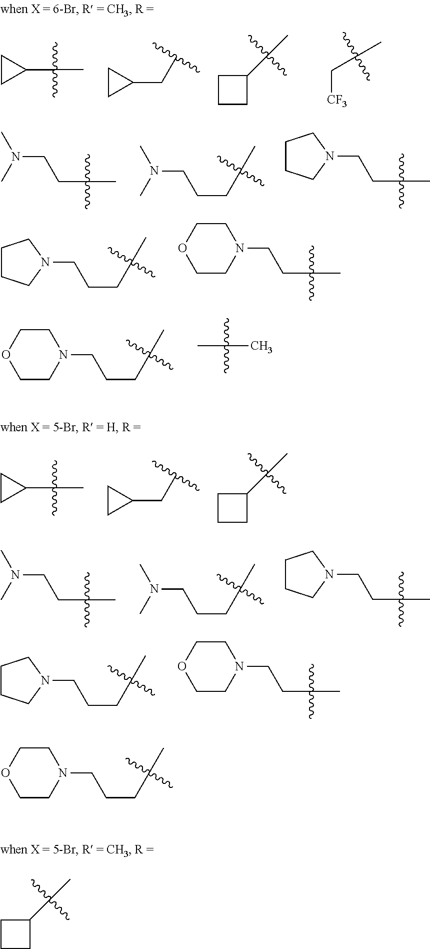

### Step 1: General Procedure for the Synthesis of Intermediate 3

To a stirred solution of 4-bromo/5-bromo-2-fluoro-1-nitrobenzene 1 (1 eq), respective amines (1 eq) in DMF (10 mL), DIPEA (2 eq) were added and stirred at room temperature for 2 h. The progress of the reaction was monitored by TLC. After completion of the reaction, the reaction mixture was quenched with water and extracted with ethyl acetate. The combined organic extracts were washed with brine, dried over anhydrous sodium sulfate and evaporated under reduced pressure. The crude product was purified by column chromatography on silica gel 100-200 mesh using 1% EtOAc-hexane to afford the intermediate 3.

| Structure | LCMS/¹H NMR |
|---|---|
| 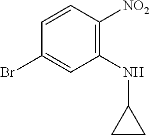 [SMILES: O=[N+]([O-])c1ccc(Br)cc1NC1CC1] | LCMS (m/z): 257.00 (M + 1) |
| 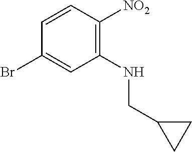 [SMILES: O=[N+]([O-])c1ccc(Br)cc1NCC1CC1] | LCMS (m/z): 271.00 (M + 1) |
| 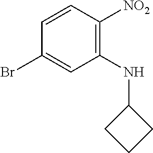 [SMILES: O=[N+]([O-])c1ccc(Br)cc1NC1CCC1] | monitored by TLC |
| 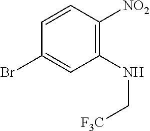 [SMILES: O=[N+]([O-])c1ccc(Br)cc1NCC(F)(F)F] | monitored by TLC |
| 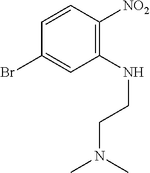 [SMILES: CN(C)CCNc1cc(Br)ccc1[N+](=O)[O-]] | LCMS (m/z): 288.10 (M + 1) |
| 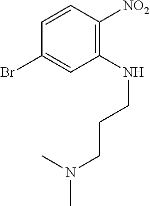 [SMILES: CN(C)CCCNc1cc(Br)ccc1[N+](=O)[O-]] | LCMS (m/z): 304.10 (M + 2) |
| 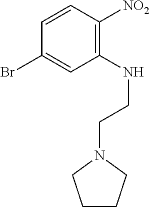 [SMILES: O=[N+]([O-])c1ccc(Br)cc1NCCN1CCCC1] | LCMS (m/z): 316.10 (M + 2) |
| 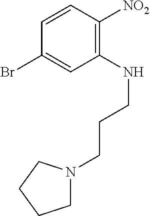 [SMILES: O=[N+]([O-])c1ccc(Br)cc1NCCCN1CCCC1] | monitored by TLC |
| 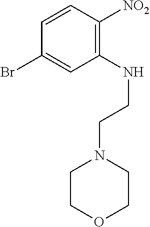 [SMILES: O=[N+]([O-])c1ccc(Br)cc1NCCN1CCOCC1] | LCMS (m/z): 330.00 (M + 1) |
| 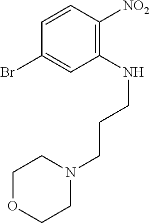 [SMILES: O=[N+]([O-])c1ccc(Br)cc1NCCCN1CCOCC1] | LCMS (m/z): 346.15 (M + 1) |
| 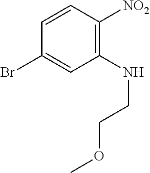 | monitored by TLC |
| 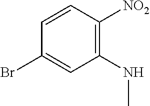 | monitored by TLC |
| 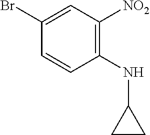 [SMILES: O=[N+]([O-])c1cc(Br)ccc1NC1CC1] | monitored by TLC |
| 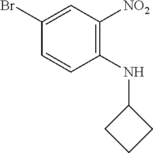 [SMILES: O=[N+]([O-])c1cc(Br)ccc1NC1CCC1] | ¹NMR (400 MHz, CDCl₃) δ: 8.30 (d, J = 2.4 Hz, 1H), 8.09 (s, 1H), 7.47-7.44 (m, 1H), 6.62 (d, J = 9.2 Hz, 1H) 4.06-4.00 (m, 1H), 2.54-2.47 (m, 2H), 2.07-1.83 (m, 4H); |
| 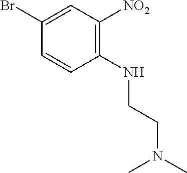 [SMILES: CN(C)CCNc1ccc(Br)cc1[N+](=O)[O-]] | monitored by TLC |
| 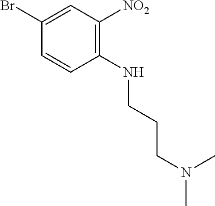 [SMILES: CN(C)CCCNc1ccc(Br)cc1[N+](=O)[O-]] | LCMS (m/z): 302.00 (M + 1) |
| 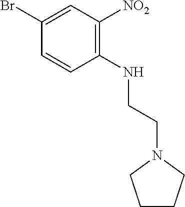 [SMILES: O=[N+]([O-])c1cc(Br)ccc1NCCN1CCCC1] | LCMS (m/z): 316.10 (M + 2) |
| 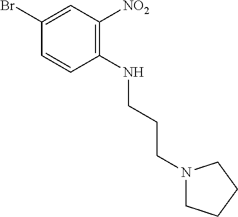 [SMILES: O=[N+]([O-])c1cc(Br)ccc1NCCCN1CCCC1] | monitored by TLC |
| 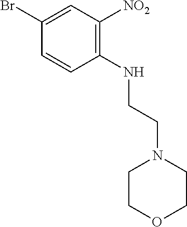 [SMILES: O=[N+]([O-])c1cc(Br)ccc1NCCN1CCOCC1] | LCMS (m/z): 332.05 (M + 2) |
| 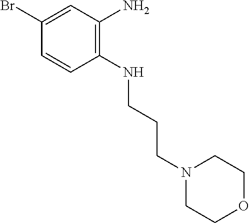 [SMILES: Nc1cc(Br)ccc1NCCCN1CCOCC1] | monitored by TLC |

### Step 2: General Procedure for the Synthesis of Intermediate (4)

To a stirred solution of respective compound 3 (1 eq), in methanol, Raney nickel (50% w/w) was added and stirred at room temperature for 18 h under hydrogen atmosphere. The progress of the reaction was monitored by TLC. After completion of the reaction, the reaction mixture was filtered and evaporated under reduced pressure to afford the following intermediates 4.

| Structure | LCMS |
|---|---|
| 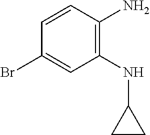 [SMILES: Nc1ccc(Br)cc1NC1CC1] | LCMS (m/z): 227.15 (M + 1) |
| 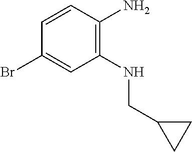 [SMILES: Nc1ccc(Br)cc1NCC1CC1] | LCMS (m/z): 240.95 (M + 1). |
| 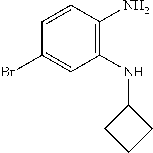 [SMILES: Nc1ccc(Br)cc1NC1CCC1] | LCMS (m/z): 243.10 (M + 2). |
| 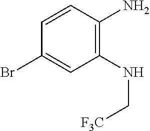 [SMILES: Nc1ccc(Br)cc1NCC(F)(F)F] | LCMS (m/z): 270.95 (M + 2). |
| 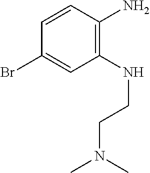 | LCMS (m/z): 260.05 (M + 2) |
| 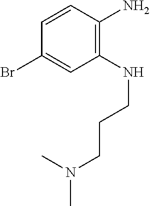 | LCMS (m/z): 274.10 (M + 1). |
| 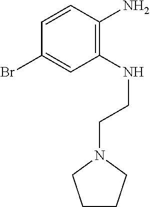 [SMILES: Nc1ccc(Br)cc1NCCN1CCCC1] | LCMS (m/z): 284.10 (M + 1) |
| 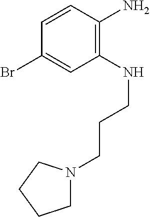 [SMILES: Nc1ccc(Br)cc1NCCCN1CCCC1] | LCMS (m/z): 300.15 (M + 2) |
| 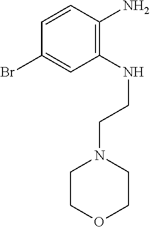 [SMILES: Nc1ccc(Br)cc1NCCN1CCOCC1] | LCMS (m/z): 300.05 (M + 1) |
| 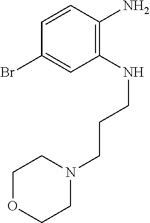 [SMILES: Nc1ccc(Br)cc1NCCCN1CCOCC1] | LCMS (m/z): 314.10 (M + 1) |
| 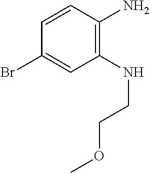 | LCMS (m/z): 246.95 (M + 2) |
| 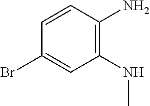 | LCMS (m/z): 200.90 (M + 1) |
| 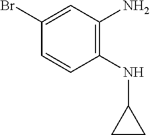 [SMILES: Nc1cc(Br)ccc1NC1CC1] | LCMS (m/z): 227.00 (M+) |
| 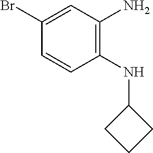 [SMILES: Nc1cc(Br)ccc1NC1CCC1] | LCMS (m/z): 241.05 (M + 1) |
| 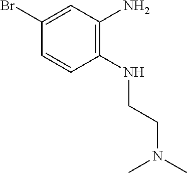 | LCMS (m/z): 258.05 (M + 1) |
| 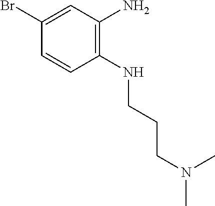 | LCMS (m/z): 272.05 (M + 1) |
| 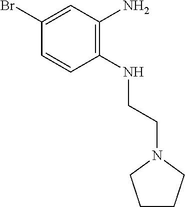 [SMILES: Nc1cc(Br)ccc1NCCN1CCCC1] | monitored by TLC |
| 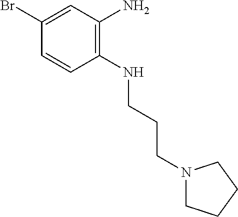 [SMILES: Nc1cc(Br)ccc1NCCCN1CCCC1] | LCMS (m/z): 300.15 (M + 2) |
| 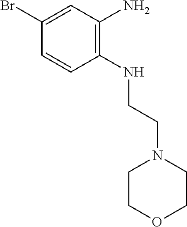 [SMILES: Nc1cc(Br)ccc1NCCN1CCOCC1] | LCMS (m/z): 300.10 (M+) |
| 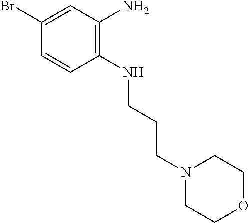 [SMILES: Nc1cc(Br)ccc1NCCCN1CCOCC1] | LCMS (m/z): 314.10 (M + 2) |

### Step 3: General Procedure

To a stirred solution of the respective compound 4 (1 eq), in DMF, trimethyl orthoformate (15 eq) and Conc. HCl (cat.,) were added and stirred at room temperature for 3 h. The progress of the reaction was monitored by TLC. After completion of the reaction, the reaction mixture was diluted with water and extracted with ethyl acetate. The combined organic extracts were dried over anhydrous sodium sulfate and evaporated under reduced pressure. The crude product was purified by column chromatography on silica gel 100-200 mesh using 30% EtOAc-hexane to afford the following intermediates.

| Structure | LCMS |
|---|---|
| 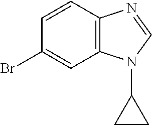 [SMILES: Brc1ccc2ncn(C3CC3)c2c1] | LCMS (m/z): 238.90 (M + 2) |
| 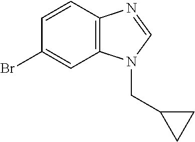 [SMILES: Brc1ccc2ncn(CC3CC3)c2c1] | LCMS (m/z): 252.95 (M + 2) |
| 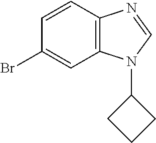 [SMILES: Brc1ccc2ncn(C3CCC3)c2c1] | LCMS (m/z): 251.05 (M + 1) |
| 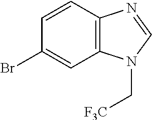 [SMILES: FC(F)(F)Cn1cnc2ccc(Br)cc21] | LCMS (m/z): 280.90 (M + 2) |
| 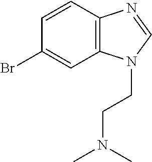 | LCMS (m/z): 270.05 (M + 2) |
| 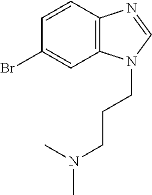 [SMILES: CN(C)CCCn1cnc2ccc(Br)cc21] | LCMS (m/z): 282.00 (M + 1) |
| 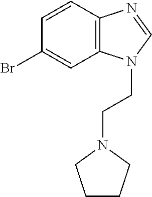 [SMILES: Brc1ccc2ncn(CCN3CCCC3)c2c1] | LCMS (m/z): 296.00 (M + 2) |
| 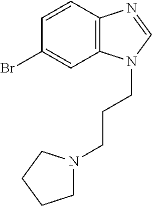 [SMILES: Brc1ccc2ncn(CCCN3CCCC3)c2c1] | LCMS (m/z): 308.05 (M + 1) |
| 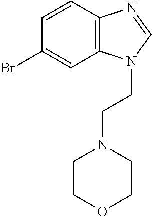 [SMILES: Brc1ccc2ncn(CCN3CCOCC3)c2c1] | LCMS (m/z): 310.00 (M + 1) |
| 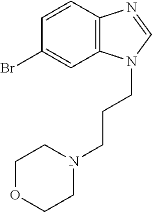 [SMILES: Brc1ccc2ncn(CCCN3CCOCC3)c2c1] | LCMS (m/z): 326.00 (M + 2) |
| 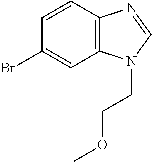 | LCMS (m/z): 277.00 (M + 23) |
| 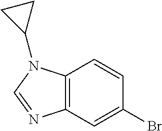 [SMILES: Brc1ccc2c(c1)ncn2C1CC1] | LCMS (m/z): 238.00 (M + 1) |
| 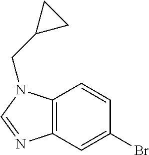 [SMILES: Brc1ccc2c(c1)ncn2CC1CC1] | LCMS (m/z): 252.95 (M + 1) |
| 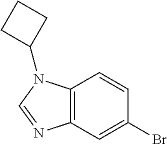 [SMILES: Brc1ccc2c(c1)ncn2C1CCC1] | LCMS (m/z): 252.95 (M + 1) |
| 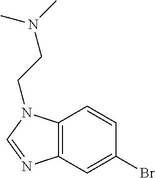 | LCMS (m/z): 268.05 (M+) |
| 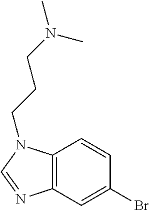 [SMILES: CN(C)CCCn1cnc2cc(Br)ccc21] | LCMS (m/z): 284.00 (M + 2) |
| 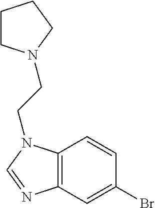 [SMILES: Brc1ccc2c(c1)ncn2CCN1CCCC1] | LCMS (m/z): 294.10 (M + 1) |
| 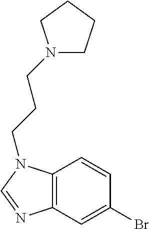 [SMILES: Brc1ccc2c(c1)ncn2CCCN1CCCC1] | LCMS (m/z): 310.10 (M + 2) |
| 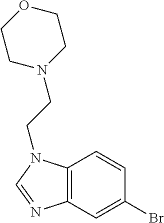 [SMILES: Brc1ccc2c(c1)ncn2CCN1CCOCC1] | LCMS (m/z): 312.10 (M + 2) |
| 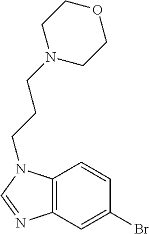 [SMILES: Brc1ccc2c(c1)ncn2CCCN1CCOCC1] | LCMS (m/z): 324.10 (M + 1) |

### General Procedure

The respective compound 4 (1 eq) was taken in CH₃COOH and heated at 110° C. for 12 h. The progress of the reaction was monitored by TLC. After completion of the reaction, the reaction mixture was neutralized with satd. sodium bicarbonate and extracted with ethyl acetate. The combined organic extracts were dried over anhydrous sodium sulfate and evaporated under reduced pressure. The crude product was purified by column chromatography on silica gel 100-200 mesh using 50% EtOAc-hexane to afford the following intermediates.

| Structure | LCMS |
|---|---|
| 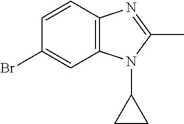 | LCMS (m/z): 253.00 (M + 2). |
| 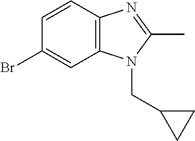 | LCMS (m/z): 267.00 (M + 2) |
| 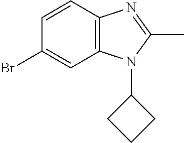 | LCMS (m/z): 265.20(M + 1). |
| 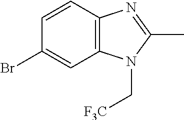 [SMILES: Cc1nc2ccc(Br)cc2n1CC(F)(F)F] | LCMS (m/z): 295.05 (M + 2). |
| 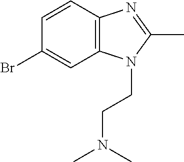 [SMILES: Cc1nc2ccc(Br)cc2n1CCN(C)C] | LCMS (m/z): 28.10 (M + 2). |
| 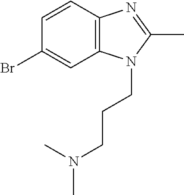 [SMILES: Cc1nc2ccc(Br)cc2n1CCCN(C)C] | LCMS (m/z): 298.10 (M + 2). |
| 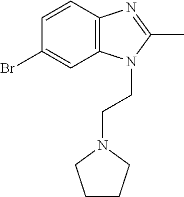 [SMILES: Cc1nc2ccc(Br)cc2n1CCN1CCCC1] | LCMS (m/z): 308.15(M + 1) |
| 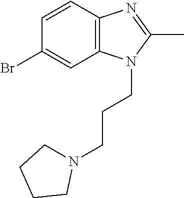 [SMILES: Cc1nc2ccc(Br)cc2n1CCCN1CCCC1] | LCMS (m/z): 322.10 (M + 1) |
| 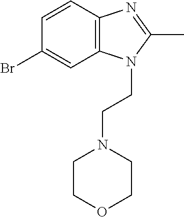 [SMILES: Cc1nc2ccc(Br)cc2n1CCN1CCOCC1] | LCMS (m/z): 324.10 (M + 1) |
| 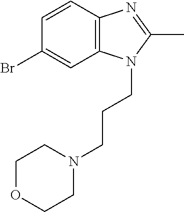 [SMILES: Cc1nc2ccc(Br)cc2n1CCCN1CCOCC1] | LCMS (m/z): 338.15 (M + 1) |
| 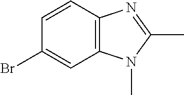 | LCMS (m/z): 224.95 (M + 1) |
| 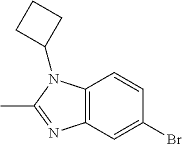 | LCMS (m/z): 267.05 (M + 2) |

### Step 4: General Procedure

The following intermediates were synthesized by following the general procedure described above for Boronate ester preparation by using the respective bromo intermediates.

| Structure | LCMS |
|---|---|
| 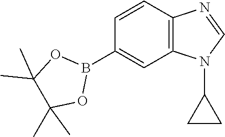 [SMILES: CC1(C)OB(c2ccc3ncn(C4CC4)c3c2)OC1(C)C] | LCMS (m/z): 285.10 (M + 1). |
| 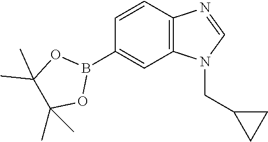 [SMILES: CC1(C)OB(c2ccc3ncn(CC4CC4)c3c2)OC1(C)C] | LCMS (m/z): 299.15 (M + 1) |
| 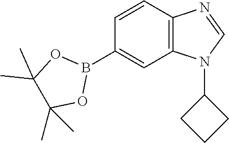 [SMILES: CC1(C)OB(c2ccc3ncn(C4CCC4)c3c2)OC1(C)C] | LCMS (m/z): 299.25 (M + 1) |
| 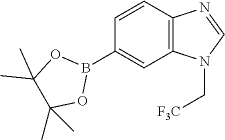 [SMILES: CC1(C)OB(c2ccc3ncn(CC(F)(F)F)c3c2)OC1(C)C] | LCMS (m/z): 327.05 (M + 1) |
| 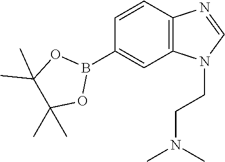 [SMILES: CN(C)CCn1cnc2ccc(B3OC(C)(C)C(C)(C)O3)cc21] | LCMS (m/z): 316.20 (M + 1) |
| 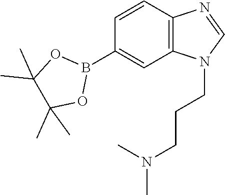 [SMILES: CN(C)CCCn1cnc2ccc(B3OC(C)(C)C(C)(C)O3)cc21] | LCMS (m/z): 330.25 (M + 1) |
| 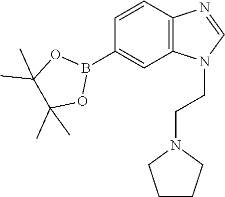 [SMILES: CC1(C)OB(c2ccc3ncn(CCN4CCCC4)c3c2)OC1(C)C] | LCMS (m/z): 342.20 (M + 1) |
| 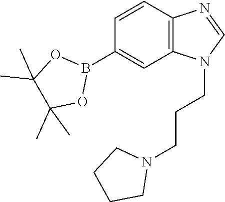 [SMILES: CC1(C)OB(c2ccc3ncn(CCCN4CCCC4)c3c2)OC1(C)C] | LCMS (m/z): 356.25 (M + 1) |
| 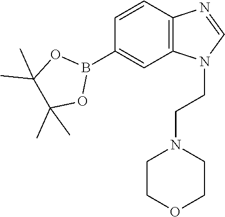 [SMILES: CC1(C)OB(c2ccc3ncn(CCN4CCOCC4)c3c2)OC1(C)C] | LCMS (m/z): 358.25 (M + 1) |
| 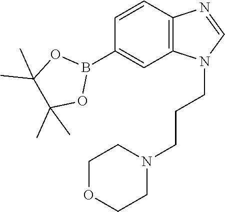 [SMILES: CC1(C)OB(c2ccc3ncn(CCCN4CCOCC4)c3c2)OC1(C)C] | LCMS (m/z): 372.25 (M + 1) |
| 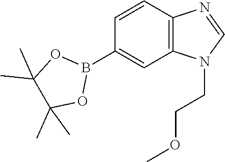 [SMILES: COCCn1cnc2ccc(B3OC(C)(C)C(C)(C)O3)cc21] | LCMS (m/z): 303.15 (M + 1) |
| 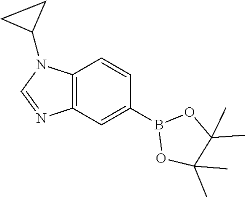 [SMILES: CC1(C)OB(c2ccc3c(c2)ncn3C2CC2)OC1(C)C] | LCMS (m/z): 285.00 (M + 1) |
| 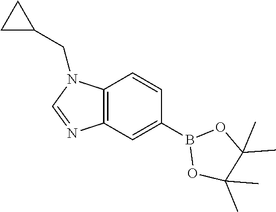 [SMILES: CC1(C)OB(c2ccc3c(c2)ncn3CC2CC2)OC1(C)C] | LCMS (m/z): 299.15 (M + 1) |
| 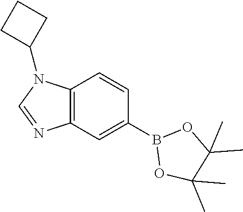 [SMILES: CC1(C)OB(c2ccc3c(c2)ncn3C2CCC2)OC1(C)C] | LCMS (m/z): 299.15 (M + 1) |
| 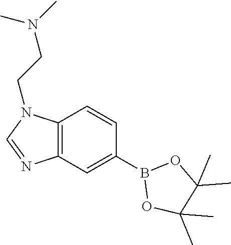 | LCMS (m/z): 316.25 (M + 1) |
| 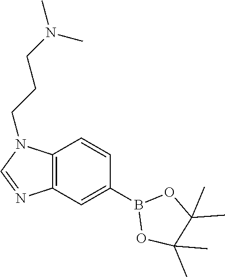 [SMILES: CN(C)CCCn1cnc2cc(B3OC(C)(C)C(C)(C)O3)ccc21] | monitored by TLC |
| 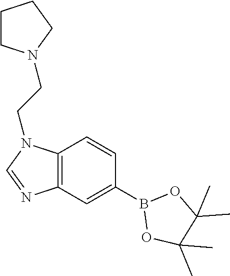 [SMILES: CC1(C)OB(c2ccc3c(c2)ncn3CCN2CCCC2)OC1(C)C] | monitored by TLC |
| 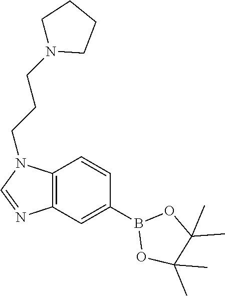 [SMILES: CC1(C)OB(c2ccc3c(c2)ncn3CCCN2CCCC2)OC1(C)C] | LCMS (m/z): 356.35 (M + 1) |
| 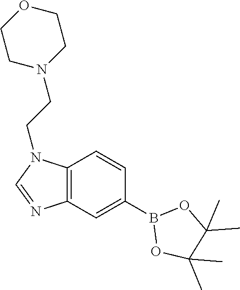 | LCMS (m/z): 372.35 (M + 1) |
| 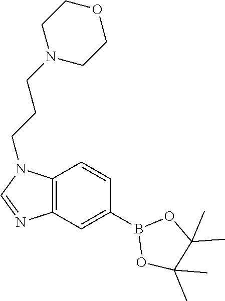 | LCMS (m/z): 386.30 (M + 1) |
| 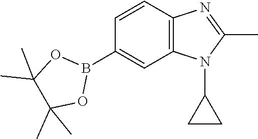 | LCMS (m/z): 299.15(M + 1). |
| 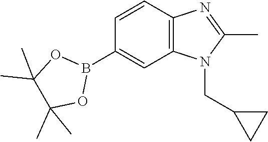 [SMILES: Cc1nc2ccc(B3OC(C)(C)C(C)(C)O3)cc2n1CC1CC1] | LCMS (m/z): 313.15(M + 1). |
| 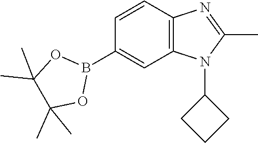 | LCMS (m/z): 313.25(M + 1). |
| 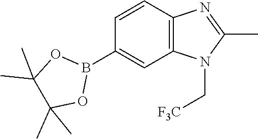 | LCMS (m/z): 341.25 (M + 1). |
| 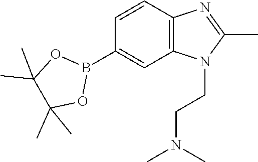 | LCMS (m/z): 330.30 (M + 1). |
| 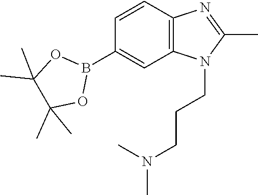 | LCMS (m/z): 344.35 (M + 1). |
| 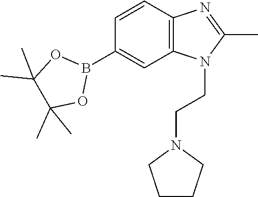 | LCMS (m/z): 356.35(M + 1) |
| 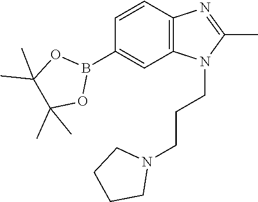 | LCMS (m/z): 370.35(M + 1) |
| 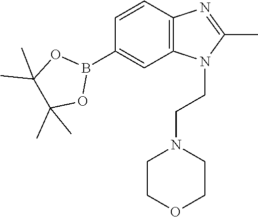 | monitored by TLC |
| 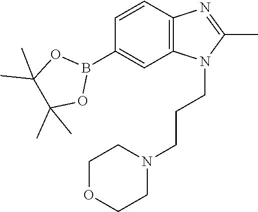 | LCMS (m/z): 386.35 (M + 1) |
| 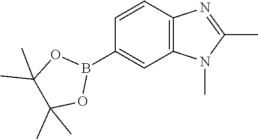 | LCMS (m/z): 273.15 (M + 1) |
| 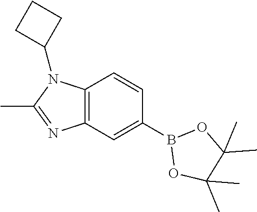 | LCMS (m/z): 313.25 (M + 1) |

### Examples 237-252

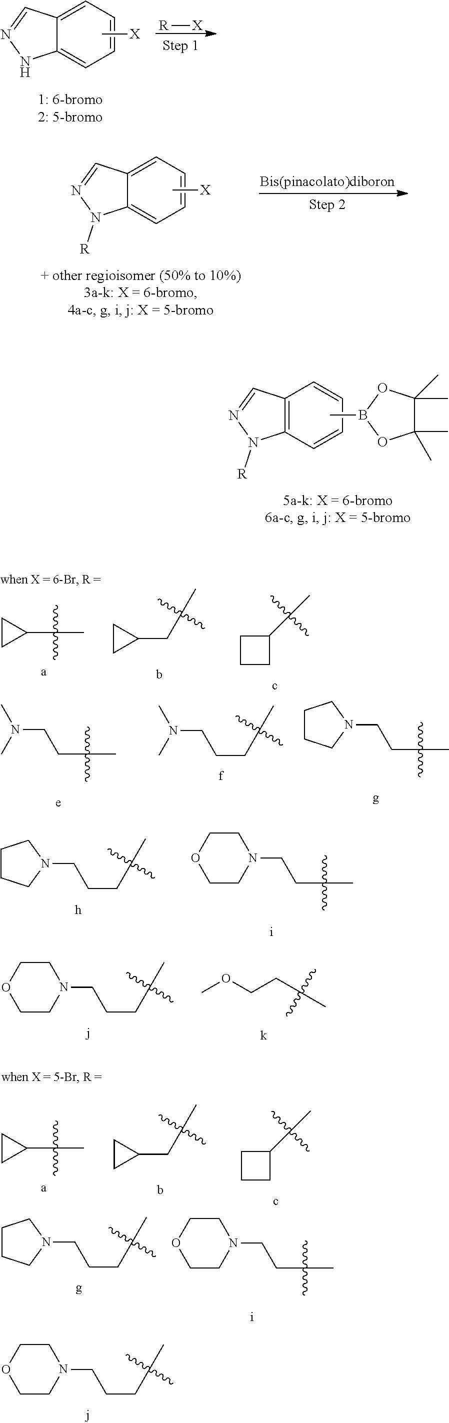

### Step 1: Procedure for alkylations on 5-bromo and 6-bromo indazoles

### i) Alkylation via Chan-Lam coupling (3a & 4a)

To a stirred solution of 6-bromo indazole/5-bromo indazole 1/2 (1 eq) and corresponding boronic acid in dichloroethane, Na₂CO₃ (2 eq) was added under oxygen atmosphere and stirred for 5 min followed by the addition of hot solution of copper acetate (1 eq) and pyridine (1 eq) in dichloroethane. The reaction mixture was heated to 75° C. for 18 h. The progress of the reaction was monitored by TLC. After completion of the reaction, the reaction mixture was quenched with saturated ammonium chloride solution, diluted with dichloromethane and filtered through celite. The separated organic extracts were washed with brine, dried over anhydrous sodium sulfate and evaporated under reduced pressure. The crude product was purified by column chromatography on silica gel 100-200 mesh using 20% EtOAc-hexane to afford compound 3a & 4a.

| Structure | LCMS |
|---|---|
| 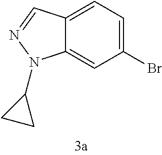 [SMILES: Brc1ccc2cnn(C3CC3)c2c1] | LCMS (m/z): 238.95(M + 2). |
| 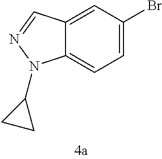 [SMILES: Brc1ccc2c(cnn2C2CC2)c1] | LCMS (m/z): 238.95(M + 2). |

### ii) Alkylation via NaH (3b-c & 4b-c)

To a stirred solution of 6-bromo indazole/5-bromo indazole 1/2 ((1 eq) in DMF, NaH (1.3 eq) was added portion wise at 0° C. and stirred for 15 min followed by the addition of alkyl halide/trifluoroethyl trifluoromethane sulfonate (1.5 eq). The reaction mixture was stirred at room temperature for 30 min. The progress of the reaction was monitored by TLC. After completion of the reaction, the reaction mixture was quenched with water, extracted with ethyl acetate (3×25 mL). The combined organic extracts were washed with brine, dried over anhydrous sodium sulfate and evaporated under reduced pressure. The crude product was purified by column chromatography on silica gel 100-200 mesh using 10% to 25% EtOAc-hexane to afford compound 3b-c & 4b-c.

| Structure | LCMS |
|---|---|
| 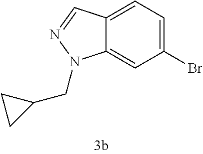 [SMILES: Brc1ccc2cnn(CC3CC3)c2c1] | LCMS (m/z): 252.95 (M + 2). |
| 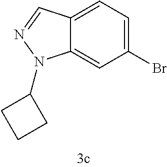 [SMILES: Brc1ccc2cnn(C3CCC3)c2c1] | monitored by TLC |
| 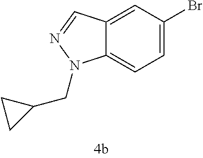 [SMILES: Brc1ccc2c(cnn2CC2CC2)c1] | LCMS (m/z): 250.95 (M + 1). |
| 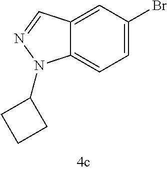 [SMILES: Brc1ccc2c(cnn2C2CCC2)c1] | LCMS (m/z): 250.90 (M + 1). |

### iii) Alkylation via K₂CO₃ (3e-i, 3k & 4i)

To a stirred solution of 6-bromo indazole/5-bromo indazole 1/2 (1 eq) in acetonitrile, K₂CO₃ (3 eq) was added followed by the addition of corresponding alkyl halide (1.5 eq). The reaction mixture was heated at 100° C. for 18 h. The progress of the reaction was monitored by TLC. After completion of the reaction, the reaction mixture was filtered and washed with ethyl acetate (3×25 mL). The combined organic extracts were evaporated under reduced pressure. The crude product was purified by column chromatography on silica gel 60-120 mesh using 10% methanol in ethyl acetate to afford compound 3e-i, 3k & 4i.

| Structure | LCMS |
|---|---|
| 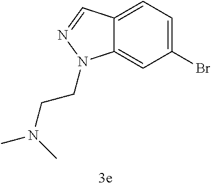 | LCMS (m/z): 270.05(M + 1). |
| 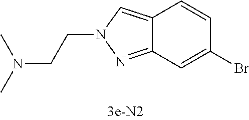 | LCMS (m/z): 270.05(M + 1). |
| 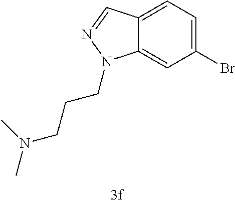 [SMILES: CN(C)CCCn1ncc2ccc(Br)cc21] | LCMS (m/z): 282.10(M + 1). |
| 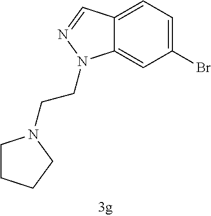 [SMILES: Brc1ccc2cnn(CCN3CCCC3)c2c1] | LCMS (m/z): 294.10(M+). |
| 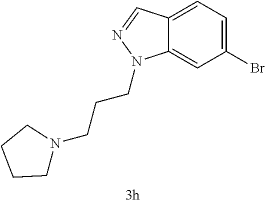 [SMILES: Brc1ccc2cnn(CCCN3CCCC3)c2c1] | LCMS (m/z): 310.00(M + 2). |
| 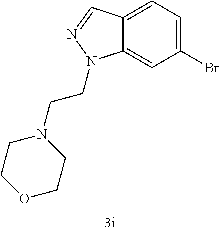 [SMILES: Brc1ccc2cnn(CCN3CCOCC3)c2c1] | LCMS (m/z): 312.00 (M + 2). |
| 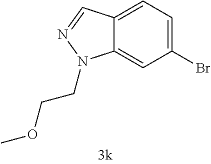 | LCMS (m/z): 256.95(M + 2). |
| 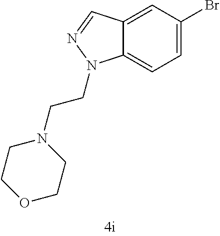 [SMILES: Brc1ccc2c(cnn2CCN2CCOCC2)c1] | LCMS (m/z): 311.95(M + 2) |

### iv) Alkylation via 3-chloro-1-bromo propane (3j, 4g & 4j)

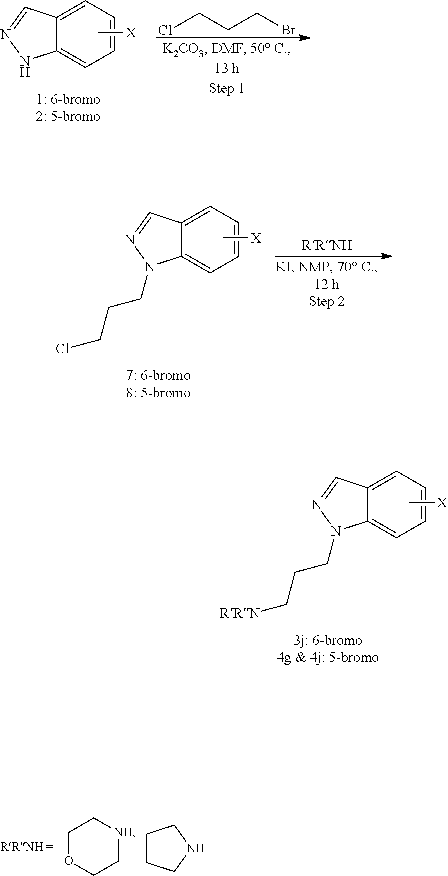

To a stirred solution of 6-bromo indazole/5-bromo indazole 1/2 (1 eq) in DMF, K₂CO₃ (3 eq) and 3-chloro-l-bromo propane (2 eq) were added and heated at 50° C. for 13 h. The progress of the reaction was monitored by TLC. After completion of the reaction, the reaction mixture was diluted with water and extracted with ethyl acetate. The combined organic extracts were evaporated under reduced pressure. The crude product was purified by column chromatography on silica gel 60-120 mesh using 20% hexane in ethyl acetate to afford compound 7/8.

To a stirred solution of compound 7/8 (1 eq) in NMP, KI (4 eq) and morpholine/pyrrolidine (6.2 eq) were added and heated at 70° C. for 12 h. After completion of the reaction, the reaction mixture was diluted with water and extracted with ethyl acetate. The combined organic extracts were evaporated under reduced pressure. The crude product was purified by column chromatography on silica gel 60-120 mesh using ethyl acetate/10% MeOH & DCM to afford compound 3j, 4g, and 4j.

| Structure | LCMS |
|---|---|
| 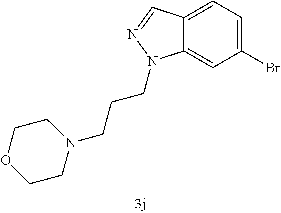 [SMILES: Brc1ccc2cnn(CCCN3CCOCC3)c2c1] | LCMS (m/z): 326.10(M + 2). |
| 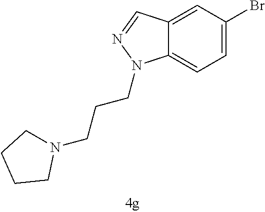 [SMILES: Brc1ccc2c(cnn2CCCN2CCCC2)c1] | LCMS (m/z): 310.10(M + 2) |
| 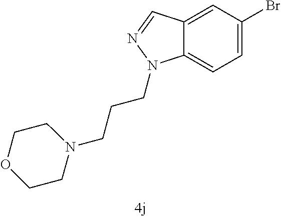 [SMILES: Brc1ccc2c(cnn2CCCN2CCOCC2)c1] | LCMS (m/z): 324.05(M + 1) |

### Step 2: Synthesis of 5 & 6 substituted indazole boronate esters (5a-c, 5e-k, 6a-c, 6g, 6i, 6j)

The following intermediates were prepared using the General Procedure for the synthesis of boronate esters using the respective bromo intermediates and bispincolato diboron.

| Structure | LCMS |
|---|---|
| 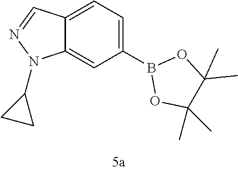 [SMILES: CC1(C)OB(c2ccc3cnn(C4CC4)c3c2)OC1(C)C] | LCMS (m/z): 285.15 (M + 1). |
| 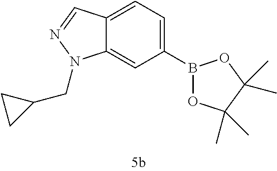 [SMILES: CC1(C)OB(c2ccc3cnn(CC4CC4)c3c2)OC1(C)C] | LCMS (m/z): 299.15 (M + 1). |
| 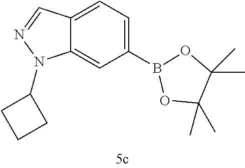 [SMILES: CC1(C)OB(c2ccc3cnn(C4CCC4)c3c2)OC1(C)C] | LCMS (m/z): 299.25 (M + 1). |
| 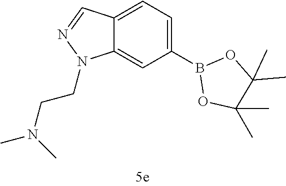 | LCMS (m/z): 316.00(M + 1). |
| 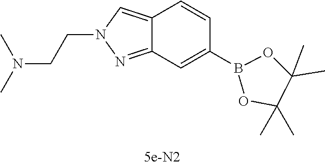 | LCMS (m/z): 316.00(M + 1). |
| 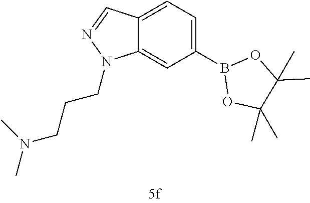 | LCMS (m/z): 330.35(M + 1). |
| 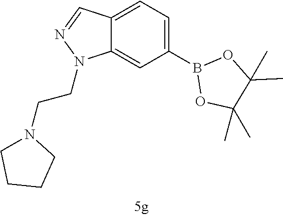 [SMILES: CC1(C)OB(c2ccc3cnn(CCN4CCCC4)c3c2)OC1(C)C] | LCMS (m/z): 342.25(M + 1). |
| 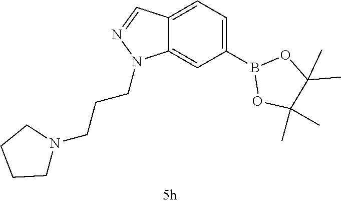 [SMILES: CC1(C)OB(c2ccc3cnn(CCCN4CCCC4)c3c2)OC1(C)C] | LCMS (m/z): 356.30(M + 1). |
| 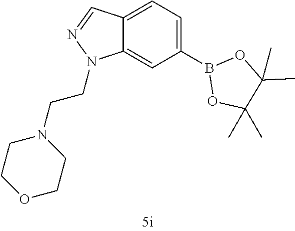 [SMILES: CC1(C)OB(c2ccc3cnn(CCN4CCOCC4)c3c2)OC1(C)C] | LCMS (m/z): 358.25(M + 1). |
| 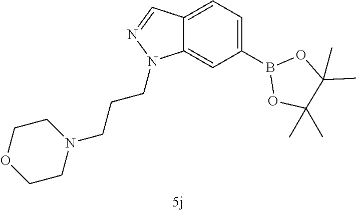 [SMILES: CC1(C)OB(c2ccc3cnn(CCCN4CCOCC4)c3c2)OC1(C)C] | LCMS (m/z): 372.30 (M + 1). |
| 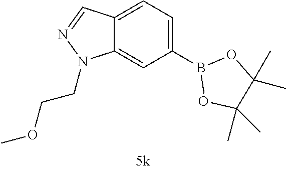 | LCMS (m/z): 303.25 (M + 1). |
| 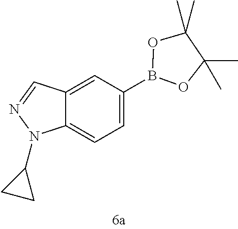 [SMILES: CC1(C)OB(c2ccc3c(cnn3C3CC3)c2)OC1(C)C] | LCMS (m/z): 285.15 (M + 1). |
| 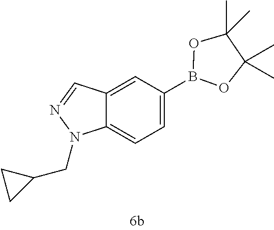 [SMILES: CC1(C)OB(c2ccc3c(cnn3CC3CC3)c2)OC1(C)C] | LCMS (m/z): 299.15 (M + 1). |
| 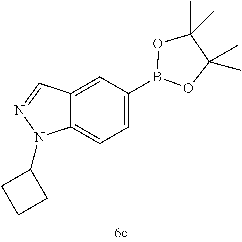 [SMILES: CC1(C)OB(c2ccc3c(cnn3C3CCC3)c2)OC1(C)C] | monitored by TLC |
| 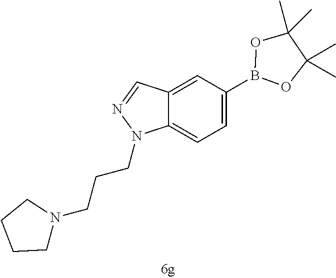 [SMILES: CC1(C)OB(c2ccc3c(cnn3CCCN3CCCC3)c2)OC1(C)C] | LCMS (m/z): 356.30(M + 1) |
| 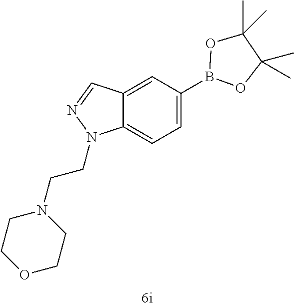 [SMILES: CC1(C)OB(c2ccc3c(cnn3CCN3CCOCC3)c2)OC1(C)C] | LCMS (m/z): 358.15(M + 1) |
| 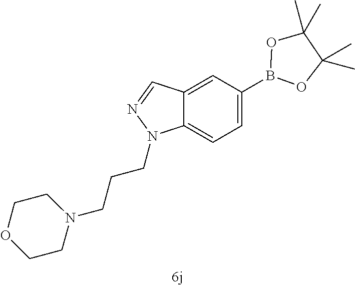 [SMILES: CC1(C)OB(c2ccc3c(cnn3CCCN3CCOCC3)c2)OC1(C)C] | LCMS (m/z): 372.25(M + 1) |

### Examples 253-262

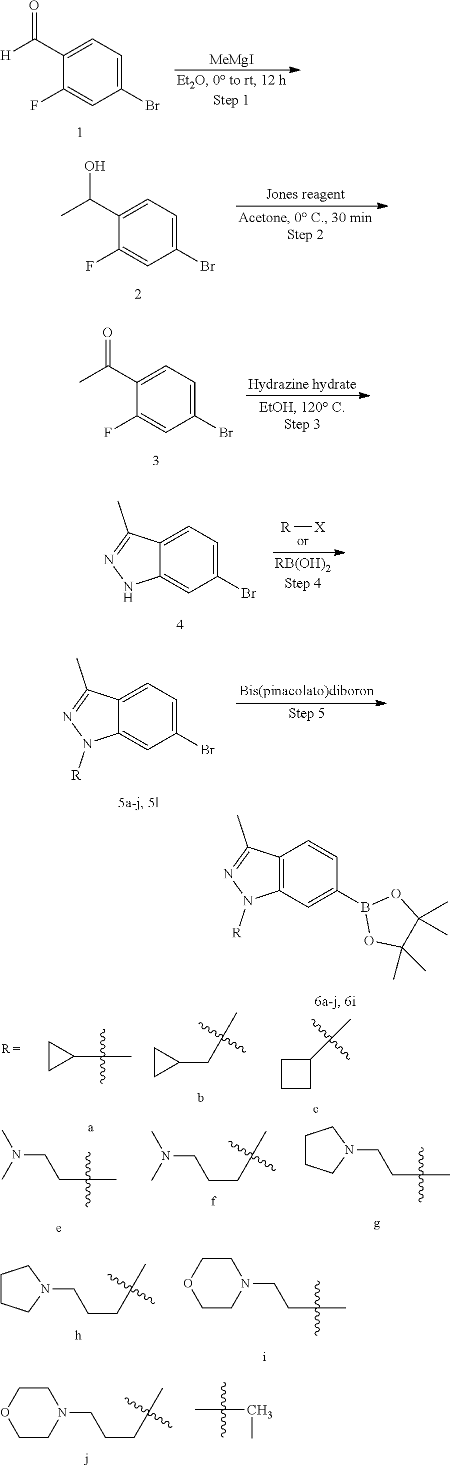

Step 1: Synthesis of 1-(4-bromo-2-fluorophenyl)ethan-1-ol (2)

A solution of compound 1 (1 eq) dissolved in dry diethyl ether was added drop wise to a stirred solution of methyl magnesium iodide (3 eq) in diethyl ether at 0° C. and stirred for 12 h at room temperature. The progress of the reaction was monitored by TLC. After completion of the reaction, the reaction mixture was cooled at 0° C. and quenched 6 N HCl solution, extracted with ethyl acetate. The combined organic extracts were washed with brine, dried over anhydrous sodium sulfate and evaporated under reduced pressure to afford compound 2. LCMS (m/z): 219.10 (M+).

### Step 2: Synthesis of 1-(4-bromo-2-fluorophenyl) ethan-1-one (3)

To a stirred solution of compound 2 (1 eq) in dry acetone, Jones reagent (1.2 mL) was added drop wise at 0° C. and stirred for 30 min at same temperature. The progress of the reaction was monitored by TLC. After completion of the reaction, the reaction mixture was quenched with water, extracted with dichloromethane. The combined organic extracts were washed with brine, dried over anhydrous sodium sulfate and evaporated under reduced pressure. The crude product was purified by column chromatography on silica gel 60-120 mesh using 2% EtOAc-hexane to afford compound 3. ¹H NMR (400 MHz, CDCl₃) δ: 7.80-7.74 (m, 1H), 7.40-7.36 (m, 2H), 2.63 (s, 3H).

### Step 3: Synthesis of 6-bromo-3-methyl-1H-indazole (4)

A stirred solution of compound 3 (1 eq) in hydrazine monohydrate (8 eq) was stirred at 120° C. for 12 h. The progress of the reaction was monitored by TLC. After completion of the reaction, the reaction mixture was diluted with water, extracted with ethyl acetate (3×25 mL). The combined organic extracts were washed with brine, dried over anhydrous sodium sulfate and evaporated under reduced pressure. The crude product was washed with diethyl ether to afford compound 4. LCMS (m/z): 213.00 (M+2).

### Step 4: General Procedure for Alkylations

### i) Alkylation via Chan-Lam Coupling (5a):

To a stirred solution of compound 4 (1 eq) and corresponding boronic acid in dichloroethane, Na₂CO₃ (2 eq) was added under oxygen atmosphere and stirred for 5 min followed by the addition of hot solution of copper acetate (1 eq) and pyridine (1 eq) in dichloroethane. The reaction mixture was heated to 75° C. for 18 h. The progress of the reaction was monitored by TLC. After completion of the reaction, the reaction mixture was quenched with saturated ammonium chloride solution, diluted with dichloromethane and filtered through celite. The separated organic extracts were washed with brine, dried over anhydrous sodium sulfate and evaporated under reduced pressure. The crude product was purified by column chromatography on silica gel 100-200 mesh using 20% EtOAc-hexane to afford compound 5a. LCMS (m/z): 251.05(M+1).

### ii) Alkylation using NaH (5b-c, 5i, 51)

To a stirred solution of compound 4 (1 eq) in DMF, NaH (1.3 eq) was added portion wise at 0° C. and stirred for 5 min followed by the addition of alkyl halide/trifluoroethyl trifluoromethyl sulfonate (1.5 eq). The reaction mixture was stirred at room temperature for 30 min. The progress of the reaction was monitored by TLC. After completion of the reaction, the reaction mixture was quenched with water, extracted with ethyl acetate (3×25 mL). The combined organic extracts were washed with brine, dried over anhydrous sodium sulfate and evaporated under reduced pressure. The crude product was purified by column chromatography on silica gel 100-200 mesh using 10% to 25% EtOAc-hexane to afford compounds 5b-c, 5i & 5I.

| Structure | LCMS |
|---|---|
| 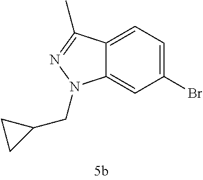 | LCMS (m/z): 265.05M + 1) |
| 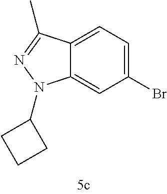 | LCMS (m/z): 265.05(M + 1) |
| 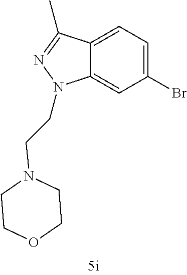 [SMILES: Cc1nn(CCN2CCOCC2)c2cc(Br)ccc12] | LCMS (m/z): 326.10(M + 1) |
| 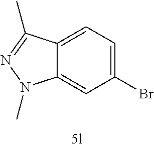 | LCMS (m/z): 227.00(M + 1) |

### iii) Alkylation using K₂CO₃ (5e & 50

To a stirred solution of compound 4 (1 eq) in acetonitrile, K₂CO₃ (3 eq) was added followed by the addition of corresponding alkyl halide (1.5 eq). The reaction mixture was heated at 100° C. for 18 h. The progress of the reaction was monitored by TLC. After completion of the reaction, the reaction mixture was filtered and washed with ethyl acetate (3×25 mL). The combined organic extracts were evaporated under reduced pressure. The crude product was purified by column chromatography on silica gel 60-120 mesh using 10% methanol in ethyl acetate to afford compounds 5e & 5f.

| Structure | LCMS |
|---|---|
| 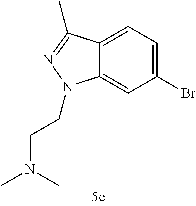 [SMILES: Cc1nn(CCN(C)C)c2cc(Br)ccc12] | LCMS (m/z): 282.10(M + 1) |
| 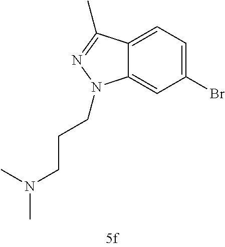 [SMILES: Cc1nn(CCCN(C)C)c2cc(Br)ccc12] | LCMS (m/z): 298.10(M + 1) |

### iv) Alkylation via 3-chloro-1-bromo propane/2-chloro-1-bromo ethane (5g, 5h & 5j)

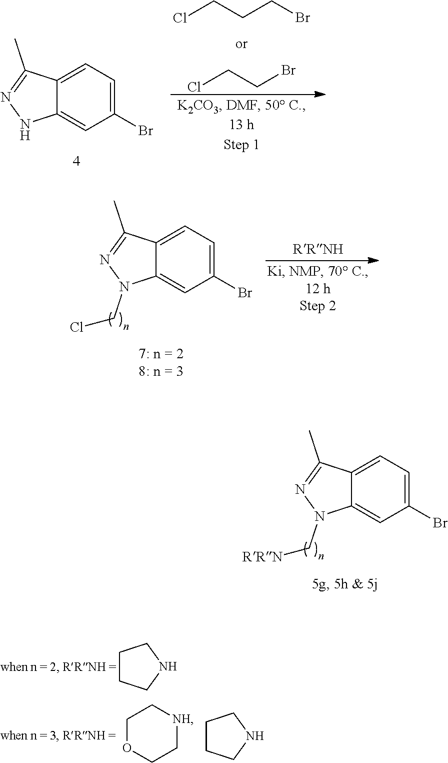

To a stirred solution of compound 4 (1 eq) in acetonitrile, K₂CO₃ (3 eq) and 3-chloro-1-bromo propane/2-chloro-1-bromoethane (2 eq) were added and heated at 80° C. for 36 h. The progress of the reaction was monitored by TLC. After completion of the reaction, the reaction mixture was diluted with water and extracted with ethyl acetate. The combined organic extracts were evaporated under reduced pressure. The crude product was purified by column chromatography on silica gel 60-120 mesh using 20% hexane in ethyl acetate to afford compound 7/8.

To a stirred solution of compound 7/8 (1 eq) in NMP, KI (4 eq) and morpholine/pyrrolidine (6.2 eq) were added and heated at 80° C. for 12 h. After completion of the reaction, the reaction mixture was diluted with water and extracted with ethyl acetate. The combined organic extracts were evaporated under reduced pressure. The crude product was purified by column chromatography on silica gel 60-120 mesh using Ethyl acetate/10% MeOH & DCM in ethyl acetate to afford compound 5g, 5h & 5j.

| Structure | LCMS |
|---|---|
| 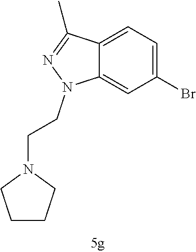 [SMILES: Cc1nn(CCN2CCCC2)c2cc(Br)ccc12] | LCMS (m/z): 308.05(M + 1) |
| 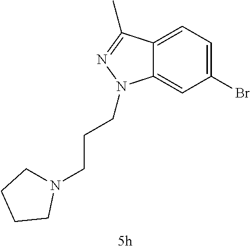 [SMILES: Cc1nn(CCCN2CCCC2)c2cc(Br)ccc12] | LCMS (m/z): 322.15(M + 1) |
| 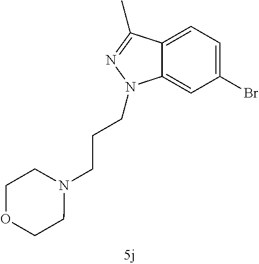 [SMILES: Cc1nn(CCCN2CCOCC2)c2cc(Br)ccc12] | LCMS (m/z): 338.15(M + 1) |

### Step 4: General Procedure for Boronate Esters (6al & 61)

The following intermediates were prepared using the General Procedure for the synthesis of boronate esters using the respective bromo compounds 5a-j, 5l and bispincolato diboron to obtain comnolincis 6a-e. 6e-j & 6l.

| Structure | LCMS |
|---|---|
| 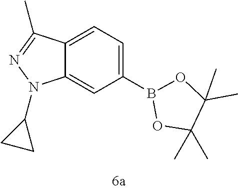 [SMILES: Cc1nn(C2CC2)c2cc(B3OC(C)(C)C(C)(C)O3)ccc12] | LCMS (m/z): 299.25(M + 1) |
| 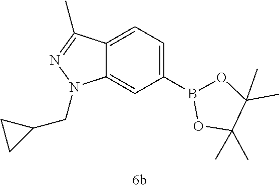 [SMILES: Cc1nn(CC2CC2)c2cc(B3OC(C)(C)C(C)(C)O3)ccc12] | LCMS (m/z): 313.30(M + 1) |
| 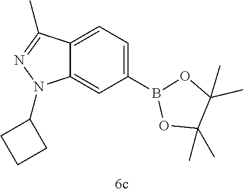 [SMILES: Cc1nn(C2CCC2)c2cc(B3OC(C)(C)C(C)(C)O3)ccc12] | LCMS (m/z): 313.25(M + 1) |
| 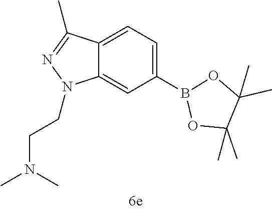 | LCMS (m/z): 330.30(M + 1) |
| 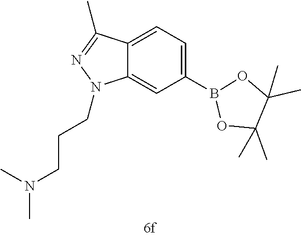 | LCMS (m/z): 344.30(M + 1) |
| 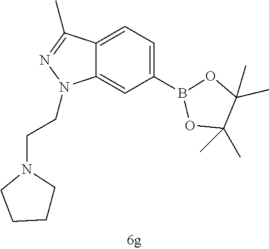 [SMILES: Cc1nn(CCN2CCCC2)c2cc(B3OC(C)(C)C(C)(C)O3)ccc12] | LCMS (m/z): 356.25(M + 1) |
| 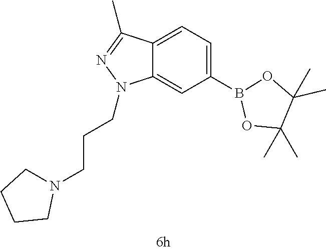 [SMILES: Cc1nn(CCCN2CCCC2)c2cc(B3OC(C)(C)C(C)(C)O3)ccc12] | LCMS (m/z): 370.40(M + 1) |
| 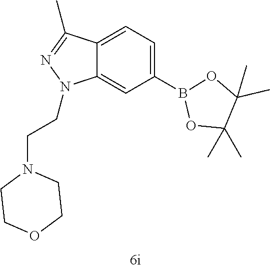 [SMILES: Cc1nn(CCN2CCOCC2)c2cc(B3OC(C)(C)C(C)(C)O3)ccc12] | LCMS (m/z): 372.35(M + 1) |
| 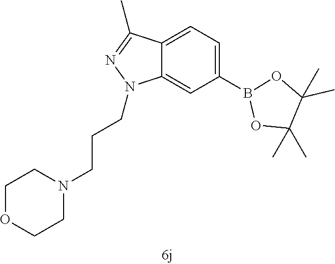 [SMILES: Cc1nn(CCCN2CCOCC2)c2cc(B3OC(C)(C)C(C)(C)O3)ccc12] | LCMS (m/z): 386.35(M + 1) |
| 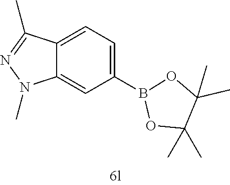 [SMILES: Cc1nn(C)c2cc(B3OC(C)(C)C(C)(C)O3)ccc12] | LCMS (m/z): 273.20(M + 1) |

### Example 263-312

### Synthesis of 5 & 6-Substituted Benzimidazoles and Indazoles p-chloro Anilines

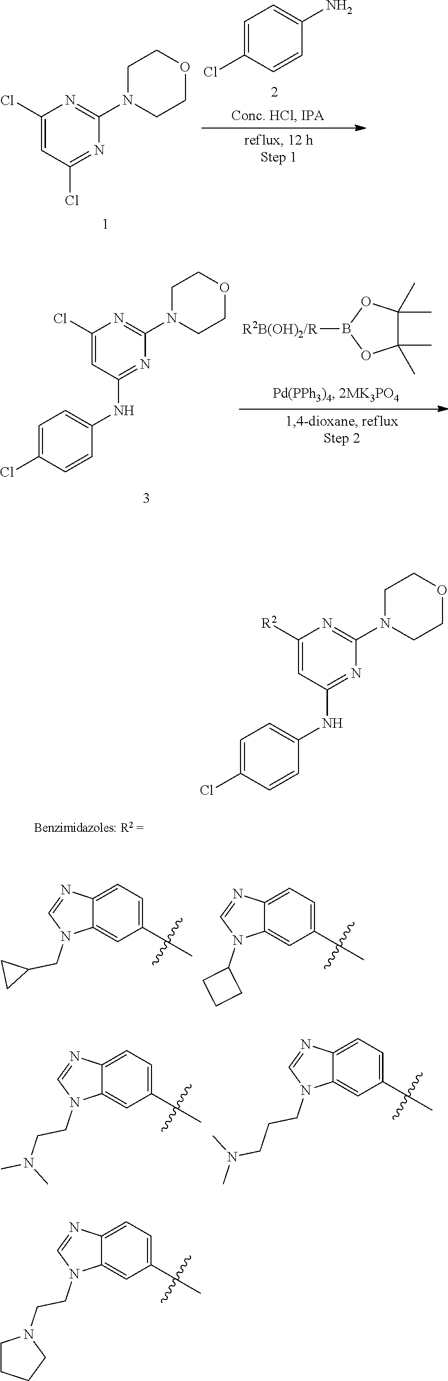

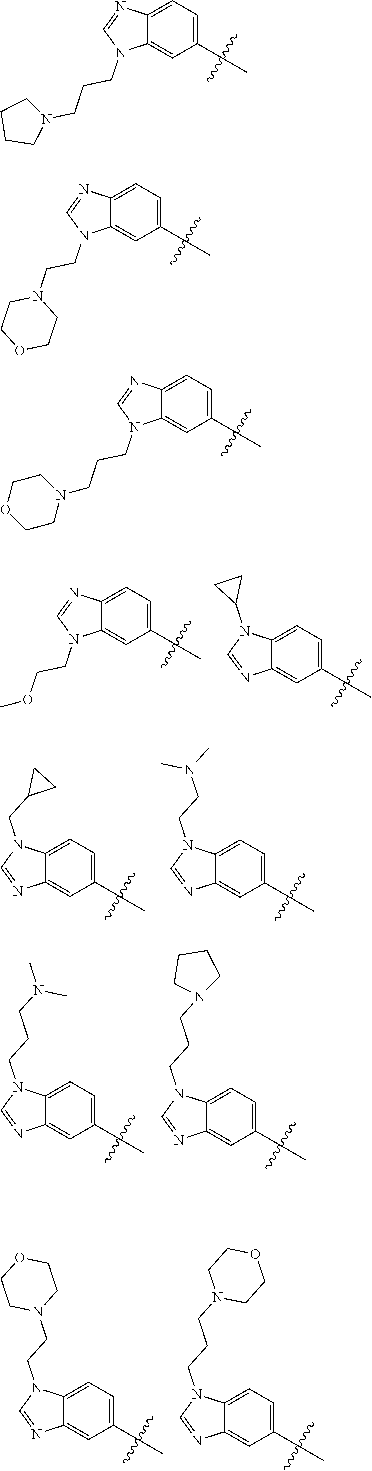

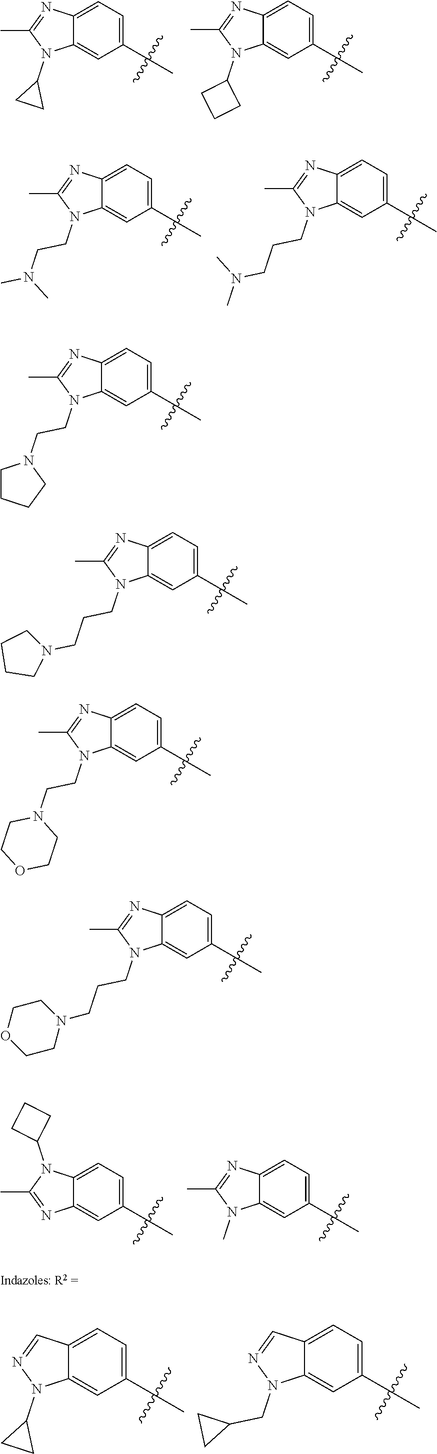

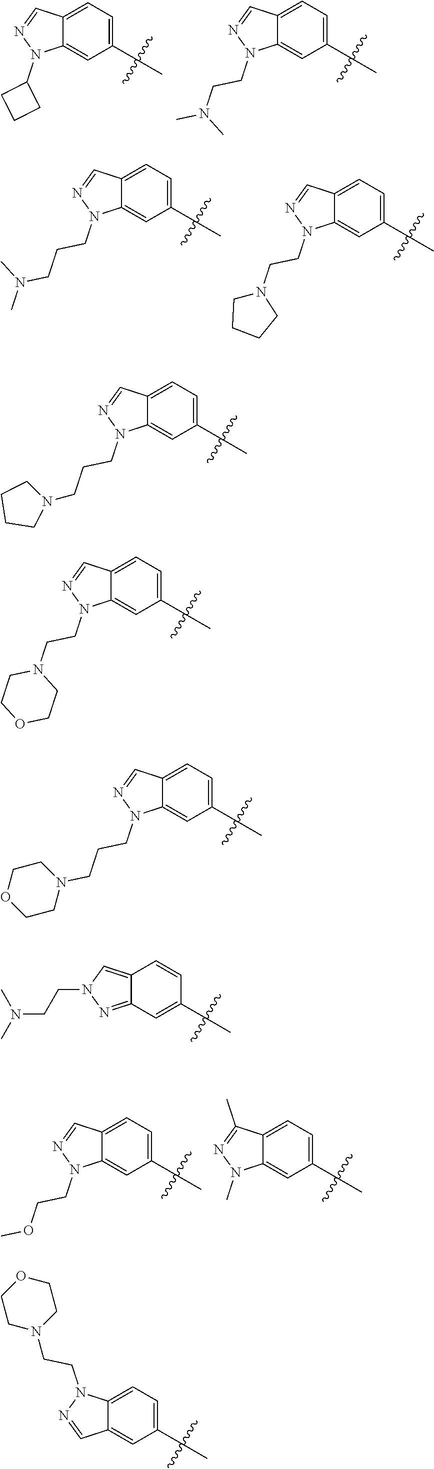

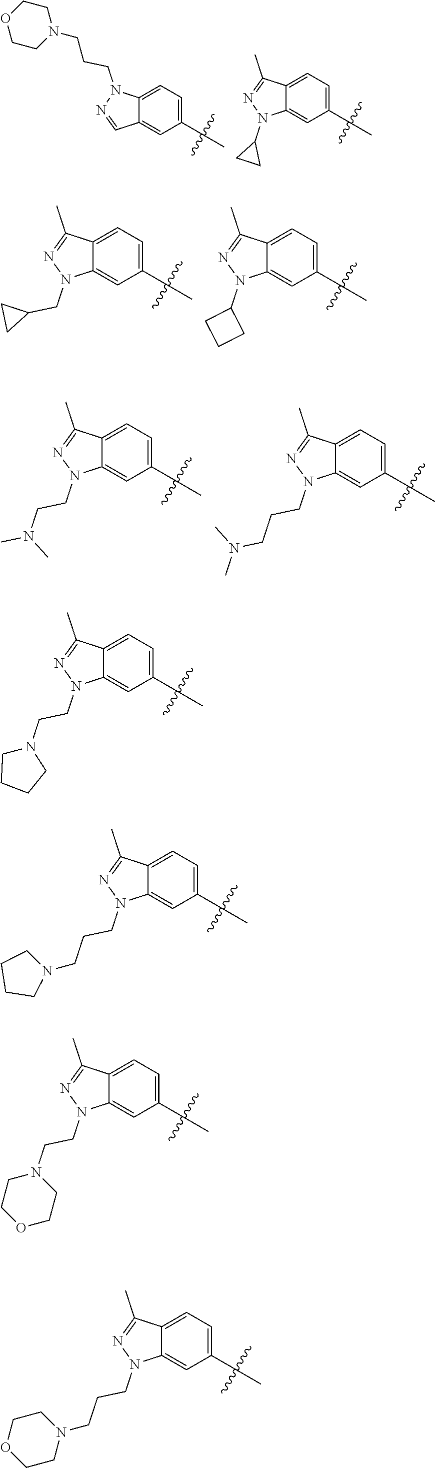

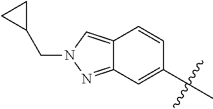 [SMILES: *c1ccc2cn(CC3CC3)nc2c1]

### Step 1: Synthesis of 6-chloro-N-(4-chlorophenyl)-2-morpholinopyrimidin-4-amine (3)

To a stirred solution of 4-(4,6-dichloropyrimidin-2-yl)morpholine 1 (1 g, 1 eq) and 4-chloro aniline 2 (0.547 g, 1 eq) in isopropanol (10 mL), Conc. HCl (2 mL) was added and heated to reflux at 100° C. for overnight. The progress of the reaction was monitored by TLC. After completion of the reaction, the reaction mixture was evaporated to dryness. The residue was taken in ethyl acetate (50 mL), washed with 1 N HCl, brine, dried over anhydrous sodium sulfate and evaporated under reduced pressure. The crude product was purified by column chromatography on silica gel 100-200 mesh using 20% EtOAc-hexane to afford the title compound 3. LCMS (m/z): 325.20 (M+1).

### Step 2: Suzuki Coupling

Adopted the general procedure as described above for Suzuki reaction using the respective pinacol boronates and compound 3.

N-(4-chlorophenyl)-6-(1-(cyclopropylmethyl)-1H-benzo[d]imidazol-6-yl)-2-morpholino pyrimidin-4-amine: ¹H NMR (400 MHz, DMSO-d₆) δ: 9.51 (s, 1H), 8.37 (s, 1H), 8.25 (d, J=1.6 Hz, 1H), 7.89 (dd, J=8.6, 1.6 Hz, 1H), 7.77-7.67 (m, 3H), 7.41-7.32 (m, 2H), 6.67 (s, 1H), 4.20 (d, J=7.1 Hz, 2H), 3.83-3.68 (m, 8H), 1.33 (ddd, J=12.4, 8.4, 4.7 Hz, 1H), 0.58 (dt, J=7.9, 2.9 Hz, 2H), 0.54-0.42 (m, 2H); HPLC purity: 99.88%; LCMS Calculated for C₂₅H₂₅ClN₆O: 460.18; Observed: 461.25 (M+1).

N-(4-chlorophenyl)-6-(1-cyclobutyl-1H-benzo[d]imidazol-6-yl)-2-morpholinopyrimidin-4-amine hydrochloride: ¹H NMR (400 MHz, DMSO-d₆) δ: 10.14 (s, 1H), 9.86 (s, 1H), 8.43 (s, 1H), 8.15 (d, J=8.6 Hz, 1H), 7.98 (d, J=8.7 Hz, 1H), 7.79-7.71 (m, 2H), 7.39 (d, J=8.7 Hz, 2H), 6.80 (s, 1H), 5.32 (p, J=8.5 Hz, 1H), 3.84-3.73 (m, 8H), 2.66 (q, J=8.0 Hz, 4H), 2.06-1.92 (m, 2H); HPLC purity: 98.25%; LCMS Calculated for C₂₅H₂₅ClN₆O: 460.18; Observed: 461.25 (M+1).

N-(4-chlorophenyl)-6-(1-(2-(dimethylamino)ethyl)-1H-benzo[d]imidazol-6-yl)-2-morpholinopyrimidin-4-amine hydrochloride: ¹H NMR (400 MHz, DMSO-d₆) δ: 11.12 (s, 1H), 10.08 (s, 1H), 9.59 (s, 1H), 8.62 (s, 1H), 8.09 (d, J=8.7 Hz, 1H), 7.97 (d, J=8.6 Hz, 1H), 7.74 (d, J=8.6 Hz, 2H), 7.40 (d, J=8.5 Hz, 2H), 6.79 (s, 1H), 5.02 (t, J=6.5 Hz, 2H), 3.88-3.74 (m, 8H), 2.88 (s, 6H); HPLC purity: 95.89%; LCMS Calculated for C₂₅H₂₈ClN₇O: 477.20; Observed: 478.30 (M+1).

N-(4-chlorophenyl)-6-(1-(3-(dimethylamino)propyl)-1H-benzo[d]imidazol-6-yl)-2-morpholinopyrimidin -4-amine: ¹H NMR (400 MHz, DMSO-d₆) δ: 9.51 (s, 1H), 8.30 (s, 1H), 8.22 (d, J=1.8 Hz, 1H), 7.88 (dd, J=8.5, 1.6 Hz, 1H), 7.72 (dd, J=8.6, 4.7 Hz, 3H), 7.41-7.32 (m, 2H), 6.66 (s, 1H), 4.34 (t, J=7.0 Hz, 2H), 3.83-3.68 (m, 8H), 2.22-2.15(m, 2H), 2.13 (s, 6H), 1.96 (p, J=6.8 Hz, 2H); HPLC purity: 99.48%; LCMS Calculated for C₂₆H₃₀ClN₇O: 491.22; Observed: 492.35 (M+1).

N-(4-chlorophenyl)-2-morpholino-6-(1-(2-(pyrrolidin-1-yl)ethyl)-1H-benzo[d]imidazol-6-yl)pyrimidin-4-amine: ¹H NMR (400 MHz, Methanol-d4) δ: 8.29 (d, J=2.0 Hz, 2H), 7.95 (dd, J=8.6, 1.6 Hz, 1H), 7.76-7.62 (m, 3H), 7.34-7.25 (m, 2H), 6.61 (s, 1H), 4.50 (t, J=6.9 Hz, 2H), 3.92-3.77 (m, 8H), 3.02 (t, J=6.8 Hz, 2H), 2.67-2.59 (m, 4H), 1.88-1.76 (m, 4H); HPLC purity: 98.19%; LCMS Calculated for C₂₇H₃₀ClN₇O: 503.22; Observed: 504.30 (M+1).

N-(4-chlorophenyl)-2-morpholino-6-(1-(3-(pyrrolidin-1-yl)propyl)-1H-benzo[d]imidazol-6-yl)pyrimidin-4-amine hydrochloride: ¹H NMR (400 MHz, DMSO-d₆) δ: 10.86 (s, 1H), 9.87 (s, 1H), 9.60 (s, 1H), 8.57 (s, 1H), 8.14 (d, J=8.7 Hz, 1H), 7.97 (d, J=8.6 Hz, 1H), 7.78-7.69 (m, 2H), 7.44-7.36 (m, 2H), 6.79 (s, 1H), 4.71 (t, J=7.1 Hz, 2H), 3.85-3.73 (m, 8H), 3.54 (dq, J=10.9, 5.5 Hz, 2H), 3.24 (dd, J=9.1, 5.4 Hz, 2H), 3.04-2.86 (m, 2H), 2.38 (p, J=6.9 Hz, 2H), 2.04-1.83 (m, 4H); HPLC purity: 98.82%; LCMS Calculated for C₂₈H₃₂ClN₇O: 517.24; Observed: 518.30 (M+1).

N-(4-chlorophenyl)-2-morpholino-6-(1-(2-morpholinoethyl)-1H-benzo[d]imidazol-6-yl)pyrimidin-4-amine hydrochloride: ¹H NMR (400 MHz, DMSO-d₆) δ: 11.98 (s, 1H), 10.25 (s, 1H), 9.65 (s, 1H), 8.68 (s, 1H), 8.08 (d, J=8.6 Hz, 1H), 7.99 (d, J=8.6 Hz, 1H), 7.79-7.70 (m, 2H), 7.46-7.37 (m, 2H), 6.81 (s, 1H), 5.08 (t, J=6.5 Hz, 2H), 3.98 (s, 2H), 3.93-3.69 (m, 12H), 3.67-3.57 (m, 2H), 3.20 (s, 2H); HPLC purity: 99.54%; LCMS Calculated for C₂₇H₃₀ClN₇O₂: 519.21; Observed: 520.40 (M+1).

N-(4-chlorophenyl)-2-morpholino-6-(1-(3-morpholinopropyl)-1H-benzo[d]imidazol-6-yl)pyrimidin-4-amine hydrochloride: ¹H NMR (400 MHz, DMSO-d₆) δ: 11.42 (s, 1H), 9.98 (s, 1H), 9.67 (s, 1H), 8.57 (s, 1H), 8.13 (d, J=8.6 Hz, 1H), 7.98 (d, J=8.7 Hz, 1H), 7.78-7.70 (m, 2H), 7.44-7.35 (m, 2H), 6.82 (s, 1H), 4.72 (t, J=6.9 Hz, 2H), 4.00-3.69 (m, 12H), 3.44 (d, J=12.3 Hz, 2H), 3.27-3.12 (m, 2H), 3.12-2.98 (m, 2H), 2.44 (q, J=7.5, 6.9 Hz, 2H); HPLC purity: 99.2%; LCMS Calculated for C₂₈H₃₂ClN₇O₂: 533.23; Observed: 534.45 (M+1).

N-(4-chlorophenyl)-6-(1-(2-methoxyethyl)-1H-benzo[d]imidazol-6-yl)-2-morpholinopyrimidin-4-amine: ¹H NMR (400 MHz, DMSO-d₆) δ: 10.05 (s, 1H), 9.62 (s, 1H), 8.56 (s, 1H), 8.16 (d, J=8.6 Hz, 1H), 7.98 (d, J=8.6 Hz, 1H), 7.78-7.69 (m, 2H), 7.39 (d, J=8.7 Hz, 2H), 6.75 (s, 1H), 4.76 (t, J=4.9 Hz, 2H), 3.82 (p, J=6.1, 5.6 Hz, 6H), 3.73 (t, J=4.6 Hz, 4H), 3.29 (s, 3H); HPLC purity: 98.55%; LCMS Calculated for C₂₄H₂₅ClN₆O₂: 464.17; Observed: 465.25 (M+1).

N-(4-chlorophenyl)-6-(1-cyclopropyl-1H-benzo[d]imidazol-5-yl)-2-morpholinopyrimidin-4-amine: ¹H NMR (400 MHz, DMSO-d₆) δ: 9.47 (s, 1H), 8.33-8.27 (m, 2H), 7.96 (dd, J=8.4, 1.7 Hz, 1H), 7.71 (d, J=8.6 Hz, 3H), 7.41-7.32 (m, 2H), 6.64 (s, 1H), 3.82-3.68 (m, 8H), 3.55 (tt, J=7.1, 3.8 Hz, 1H), 1.17-1.01 (m, 4H).; HPLC purity: 98.68%; LCMS Calculated for C₂₄H₂₃ClN₆O: 446.16; Observed: 447.15 (M+1).

N-(4-chlorophenyl)-6-(1-(cyclopropylmethyl)-1H-benzo[d]imidazol-5-yl)-2-morpholinopyrimidin-4-amine: ¹H NMR (400 MHz, DMSO-d₆) δ: 9.85 (s, 1H), 9.66 (d, J=11.3 Hz, 1H), 8.57 (s, 1H), 8.43 (s, 1H), 8.17 (q, J=8.6 Hz, 1H), 7.98 (d, J=8.6 Hz, 1H), 7.73 (d, J=8.7 Hz, 2H), 7.39 (d, J=8.6 Hz, 2H), 6.75 (d, J=2.4 Hz, 1H), 4.42 (dd, J=23.6, 7.3 Hz, 2H), 3.80-3.73 (m, 8H), 1.51-1.39 (m, 1H), 0.73-0.51 (m, 4H); HPLC purity: 97.48%; LCMS Calculated for C₂₅H₂₅ClN₆O: 460.18; Observed: 461.25 (M+1).

N-(4-chlorophenyl)-6-(1-(2-(dimethylamino)ethyl)-1H-benzo[d]imidazol-5-yl)-2-morpholinopyrimidin-4-amine: ¹H NMR (400 MHz, DMSO-d₆) δ: 11.03 (s, 1H), 10.12 (s, 1H), 9.64 (s, 1H), 8.41 (d, J=1.5 Hz, 1H), 8.25-8.11 (m, 2H), 7.79-7.70 (m, 2H), 7.44-7.36 (m, 2H), 6.79 (s, 1H), 4.98 (t, J=6.3 Hz, 2H), 3.84-3.65 (m, 10H), 2.86 (d, J=4.1 Hz, 6H); HPLC purity: 98.94%; LCMS Calculated for C₂₅H₂₈ClN₇O: 477.20; Observed: 478.40 (M+1).

N-(4-chlorophenyl)-6-(1-(3-(dimethylamino)propyl)-1H-benzo[d]imidazol-5-yl)-2-morpholinopyrimidin-4-amine: ¹H NMR (400 MHz, DMSO-d₆) δ: 10.85 (s, 1H), 10.07 (s, 1H), 9.68 (s, 1H), 8.42 (s, 1H), 8.18 (s, 2H), 7.78-7.69 (m, 2H), 7.44-7.35 (m, 2H), 6.79 (s, 1H), 4.66 (t, J=6.9 Hz, 2H), 3.84-3.69 (m, 8H), 3.20-3.10 (m, 2H), 2.74 (d, J=4.8 Hz, 6H), 2.38 (q, J=7.5 Hz, 2H); HPLC purity: 99.41%; LCMS Calculated for C₂₆H₃₀ClN₇O: 491.22; Observed: 492.30 (M+1).

N-(4-chlorophenyl)-2-morpholino-6-(1-(2-(pyrrolidin-1-yl)ethyl)-1H-benzo[d]imidazol-5-yl)pyrimidin-4-amine: ¹H NMR (400 MHz, DMSO-d₆) δ: 9.48 (s, 1H), 8.29 (d, J=7.1 Hz, 2H), 7.92 (d, J=8.6 Hz, 1H), 7.70 (dd, J=8.5, 5.5 Hz, 3H), 7.36 (d, J=8.6 Hz, 2H), 6.63 (s, 1H), 4.38 (t, J=6.2 Hz, 2H), 3.83-3.72 (m, 8H), 2.84 (t, J=6.3 Hz, 2H), 2.54-2.46 (m, 4H), 1.69-1.60 (m, 4H); HPLC purity: 96.31%; LCMS Calculated for C₂₇H₃₀ClN₇O: 503.22; Observed: 504.40 (M+1).

N-(4-chlorophenyl)-2-morpholino-6-(1-(2-morpholinoethyl)-1H-benzo[d]imidazol-5-yl) pyrimidin-4-amine: ¹H NMR (400 MHz, DMSO-d₆) 6:11.94 (s, 1H), 10.15 (s, 1H), 9.63 (s, 1H), 8.41 (s, 1H), 8.23 (d, J=8.7 Hz, 1H), 8.14 (d, J=8.8 Hz, 1H), 7.78-7.71 (m, 2H), 7.43-7.36 (m, 2H), 6.79 (s, 1H), 5.03 (t, J=6.5 Hz, 2H), 3.99 (d, J=12.8 Hz, 2H), 3.92-3.77 (m, 6H), 3.74 (d, J=5.0 Hz, 6H), 3.59 (d, J=13.0 Hz, 2H), 3.18-3.04 (m, 2H); HPLC purity: 98.24%; LCMS Calculated for C₂₇H₃₀ClN₇O₂: 519.21; Observed: 520.45 (M+1).

N-(4-chlorophenyl)-2-morpholino-6-(1-(3-morpholinopropyl)-1H-benzo[d]imidazol-5-yl)pyrimidin-4-amine: ¹H NMR (400 MHz, DMSO-d₆) δ: 11.41 (s, 1H), 9.99 (s, 1H), 9.64 (s, 1H), 8.43 (s, 1H), 8.17 (s, 2H), 7.77-7.69 (m, 2H), 7.44-7.35 (m, 2H), 6.77 (s, 1H), 4.67 (t, J=6.9 Hz, 2H), 4.00-3.90 (m, 2H), 3.90-3.69 (m, 10H), 3.46-3.35 (m, 2H), 3.19 (dt, J=10.2, 5.6 Hz, 2H), 3.10-2.96 (m, 2H), 2.42 (p, J=7.3 Hz, 2H); HPLC purity: 97.95%; LCMS Calculated for C₂₈H₃₂ClN₇O₂: 533.23; Observed: 534.45 (M+1).

N-(4-chlorophenyl)-6-(1-cyclopropyl-2-methyl-1H-benzo[d]imidazol-6-yl)-2-morpholinopyrimidin-4-amine hydrochloride: ¹H NMR (400 MHz, DMSO-d₆) δ: 9.95 (s, 1H), 8.38 (d, J=1.5 Hz, 1H), 8.19-8.11 (m, 1H), 7.90 (d, J=8.6 Hz, 1H), 7.79-7.70 (m, 2H), 7.44-7.35 (m, 2H), 6.78 (s, 1H), 3.84-3.69 (m, 8H), 3.62 (tt, J=7.1, 4.0 Hz, 1H), 2.89 (s, 3H), 1.41-1.20 (m, 4H); HPLC purity: 94.42%; LCMS Calculated for C₂₅H₂₅ClN₆O: 460.18; Observed: 461.35 (M+1).

N-(4-chlorophenyl)-6-(1-cyclobutyl-2-methyl-1H-benzo[d]imidazol-6-yl)-2-morpholinopyrimidin-4-amine¹H NMR (400 MHz, DMSO-d₆) δ: 10.02 (s, 1H), 8.50 (s, 1H), 8.12 (d, J=8.6 Hz, 1H), 7.91 (d, J=8.6 Hz, 1H), 7.75 (d, J=8.8 Hz, 2H), 7.39 (d, J=8.5 Hz, 2H), 6.81 (s, 1H), 5.25 (p, J=8.7 Hz, 1H), 3.84-3.67 (m, 8H), 2.96-2.82 (m, 2H),2.80 (s, 3H), 2.77-2.66 (m, 2H), 2.09-1.94 (m, 2H); HPLC purity: 98.37%; LCMS Calculated for C₂₆H₂₇ClN₆O: 474.19; Observed: 475.35 (M+1).

N-(4-chlorophenyl)-6-(1-(2-(dimethylamino)ethyl)-2-methyl-1H-benzo[d]imidazol-6-yl)-2-morpholinopyrimidin-4-amine: ¹H NMR (400 MHz, DMSO-d₆) 6:11.5 (s,1H),10.92 (s,1H), 8.50 (d, J=1.5 Hz, 1H), 8.13 (dd, J=8.6, 1.5 Hz, 1H), 7.85 (d, J=8.6 Hz, 1H), 7.73-7.65 (m, 2H), 7.42-7.33 (m, 2H), 6.78 (s, 1H), 4.85 (t, J=7.7 Hz, 2H), 3.83-3.72(m,8H), 3.61 (t, J=7.7 Hz, 2H), 2.93 (s, 6H), 2.89 (s, 3H); HPLC purity: 96.59%; LCMS Calculated for C₂₆H₃₀ClN₇O: 491.22; Observed: 492.40 (M+1).

N-(4-chlorophenyl)-6-(1-(3-(dimethylamino)propyl)-2-methyl-1H-benzo[d]imidazol-6-yl)-2-morpholinopyrimidin-4-amine: ¹H NMR (400 MHz, Methanol-d4) δ: 8.73 (s, 1H), 8.03 (d, J=8.7 Hz, 1H), 7.95 (d, J=8.6 Hz, 1H), 7.66 (d, J=8.4 Hz, 2H), 7.48-7.41 (m, 2H), 6.71 (s, 1H), 4.83-4.66 (m, 2H), 3.94-3.86 (m, 8H), 3.47-3.38 (m, 2H), 2.96 (s, 3H), 2.94 (s, 6H), 2.46 (p, J=8.0 Hz, 2H); HPLC purity: 96.86%; LCMS Calculated for C₂₇H₃₂ClN₇O: 505.24; Observed: 506.35 (M+1).

N-(4-chlorophenyl)-6-(2-methyl-1-(2-(pyrrolidin-1-yl)ethyl)-1H-benzo[d]imidazol-6-yl)-2-morpholinopyrimidin-4-amine: ¹H NMR (400 MHz, DMSO-d₆) δ: 11.73 (dd, J=12.1, 6.4 Hz, 1H), 9.89 (s, 1H), 8.63 (s, 1H), 8.10 (d, J=8.6 Hz, 1H), 7.90 (d, J=8.6 Hz, 1H), 7.78-7.68 (m, 2H), 7.46-7.32 (m, 2H), 6.78 (s, 1H), 4.97 (t, J=7.1 Hz, 2H), 3.83-3.72 (m, 10H), 3.58 (dq, J=11.0, 5.4 Hz, 2H), 3.15 (dq, J=13.7, 7.2 Hz, 2H), 2.94 (s, 3H), 2.03 (td, J=11.1, 9.7, 4.3 Hz, 2H), 2.00-1.85 (m, 2H); HPLC purity: 96.71%; LCMS Calculated for C₂₈H₃₂ClN₇O: 517.24; Observed: 518.40 (M+1).

N-(4-chlorophenyl)-6-(2-methyl-1-(3-(pyrrolidin-l-yl)propyl)-1H-benzo[d]imidazol-6-yl)-2-morpholinopyrimidin-4-amine: ¹H NMR (400 MHz, DMSO-d₆) δ: 9.50 (s, 1H), 8.12 (s, 1H), 7.81 (dd, J=8.4, 1.6 Hz, 1H), 7.75-7.67 (m, 2H), 7.58 (d, J=8.4 Hz, 1H), 7.40-7.32 (m, 2H), 6.63 (s, 1H), 4.30 (t, J=6.6 Hz, 2H), 3.82-3.68 (m, 8H), 2.58 (s, 3H), 2.50-2.41 (m, 6H), 1.95 (d, J=9.1 Hz, 2H), 1.69 (d, J=6.0 Hz, 4H); HPLC purity: 98.66%; LCMS Calculated for C₂₉H₃₄ClN₇O: 531.25; Observed: 532.35 (M+1).

N-(4-chlorophenyl)-6-(2-methyl-1-(2-morpholinoethyl)-1H-benzo[d]imidazol-6-yl)-2-morpholinopyrimidin-4-amine: ¹H NMR (400 MHz, DMSO-d₆) δ: 12.38 (s, 1H), 10.05 (s, 1H), 8.69 (s, 1H), 8.09 (d, J=8.5 Hz, 1H), 7.92 (d, J=8.6 Hz, 1H), 7.78-7.70 (m, 2H), 7.40 (d, J=8.6 Hz, 2H), 6.81 (s, 1H), 5.04 (t, J=7.7 Hz, 2H), 4.03 (d, J=13.5 Hz, 4H), 3.85 (d, J=5.6 Hz, 4H), 3.76-3.55 (m, 8H), 3.24 (s, 2H), 2.94 (s, 3H); HPLC purity: 99.09%; LCMS Calculated for C₂₈H₃₂ClN₇O₂: 533.23; Observed: 534.45 (M+1).

N-(4-chlorophenyl)-6-(2-methyl-1-(3-morpholinopropyl)-1H-benzo[d]imidazol-6-yl)-2-morpholino pyrimidin-4-amine: ¹H NMR (400 MHz, Methanol-d4) δ: 8.71 (s, 1H), 8.05-7.90 (m, 2H), 7.68 (d, J=8.5 Hz, 2H), 7.50-7.39 (m, 2H), 6.77 (s, 1H), 4.70 (t, J=7.7 Hz, 2H), 4.10-4.01 (m, 2H), 3.90 (dt, J=31.8, 4.5 Hz, 10H), 3.56 (d, J=12.1 Hz, 2H), 3.51-3.40 (m, 2H), 3.32-3.16 (m, 2H), 3.01 (s, 3H), 2.55 (d, J=15.0 Hz, 2H); HPLC purity: 98.45%; LCMS Calculated for C₂₉H₃₄ClN₇O₂: 547.25; Observed: 548.35 (M+1).

N-(4-chlorophenyl)-6-(1,2-dimethyl-1H-benzo[d]imidazol-6-yl)-2-morpholinopyrimidin-4-amine: ¹H NMR (400 MHz, DMSO-d₆) δ: 9.80(s, 1H), 8.48 (s, 1H), 8.14 (d, J=8.6 Hz, 1H), 7.89 (d, J=8.6 Hz, 1H), 7.73 (d, J=8.4 Hz, 2H), 7.38 (d, J=8.2 Hz, 2H), 6.75 (s, 1H), 4.18 (s, 3H), 3.81-3.73 (m,8H), 2.86 (s, 3H); HPLC purity: 98.15%; LCMS Calculated for C₂₃H₂₃ClN₆O: 434.16; Observed: 435.25 (M+1).

N-(4-chlorophenyl)-6-(1-cyclobutyl-2-methyl-1H-benzo[d]imidazol-5-yl)-2-morpholinopyrimidin-4-amine: ¹H NMR (400 MHz, DMSO-d₆) δ: 9.47 (s, 1H), 8.16 (s, 1H), 7.90-7.76 (m, 2H), 7.71 (d, J=8.5 Hz, 2H), 7.36 (d, J=8.4 Hz, 2H), 6.61 (s, 1H), 5.01 (p, J=8.9 Hz, 1H), 3.82-3.72 (m, 8H), 2.79 (dt, J=12.6, 9.2 Hz, 2H), 2.58 (s, 3H),2.55 (d, J=7.7 Hz, 2H), 2.02-1.82 (m, 2H); HPLC purity: 98.81%; LCMS Calculated for C₂₆H₂₇ClN₆O: 474.19; Observed: 475.40 (M+1).

N-(4-chlorophenyl)-6-(1-cyclopropyl-1H-indazol-6-yl)-2-morpholinopyrimidin-4-amine: ¹H NMR (400 MHz, DMSO-d₆) δ: 10.15 (s, 1H),8.24 (s, 1H),8.09(s, 1H), 7.88 (d, J=8.4 Hz, 1H), 7.74-7.72(m, 3H), 7.41 (d, J=8.8, 2H), 6.73 (s, 1H), 3.87-3.73 (m, 9H),1.17-1.16(m,4H); HPLC purity: 92.30%; LCMS Calculated for C₂₄H₂₃ClN₆O: 446.16; Observed: 447.20 (M+1).

N-(4-chlorophenyl)-6-(1-(cyclopropylmethyl)-1H-indazol-6-yl)-2-morpholinopyrimidin-4-amine: ¹H NMR (400 MHz, CD3OD) δ: 8.15 (s, 2H), 7.98 (d, J=8.8 Hz, 1H), 7.66(s, 2H), 7.51 (d, J=8.4, 1H), 7.45 (d, J=8.4 Hz, 2H), 6.61 (s, 1H),4.43 (d, J=6.8 Hz, 2H), 3.87-3.86 (m, 8H),1.46-1.40(m,1H), 0.59-0.56 (m,2H), 0.50-0.48 (m,2H) ; HPLC purity: 99.74%; LCMS Calculated for C₂₅H₂₅ClN₆O: 460.18; Observed: 461.20 (M+1).

N-(4-chlorophenyl)-6-(2-(cyclopropylmethyl)-2H-indazol-6-yl)-2-morpholino pyrimidin-4-amine: The title compound has been synthesized by following the General Procedure for Suzuki Coupling described above using compound 7 and Boronate ester 5. ¹H NMR (400 MHz, MeOD) δ: 8.54 (s, 1H),8.13 (s, 1H), 7.98 (d, J=8.4 Hz, 1H), 7.66 (s, 2H), 7.48-7.42 (m, 3H), 6.60 (s, 1H), 4.41 (d, J=7.2 Hz, 2H), 3.90-3.84 (m, 8H), 1.52-1.46 (m, 1H),0.73-0.68 (m, 2H),0.56-0.52 (m, 2H); HPLC purity: 98.38%; LCMS Calculated for C₂₅H₂₅ClN₆O: 460.18; Observed: 461.15 (M+1).

N-(4-chlorophenyl)-6-(1-cyclobutyl-1H-indazol-6-yl)-2-morpholinopyrimidin-4-amine: ¹H NMR (400 MHz, DMSO-d6) δ: 10.31 (s, 1H), 8.24 (d, J=2.9 Hz, 2H), 7.92 (d, J=8.4 Hz, 1H), 7.71 (d, J=8.5 Hz, 2H), 7.60 (d, J=8.4 Hz, 1H), 7.45 (d, J=8.4 Hz, 2H), 6.67 (s, 1H), 5.41 (p, J=8.2 Hz, 1H), 3.86-3.75 (m, 8H), 2.75-2.60 (m, 2H), 2.50-2.40 (m, 2H), 1.91 (td, J=9.5, 4.9 Hz, 2H); HPLC purity: 96.50%; LCMS Calculated for C₂₅H₂₅ClN₆O: 460.18; Observed: 461.25 (M+1).

N-(4-chlorophenyl)-6-(1-(2-(dimethylamino)ethyl)-1H-indazol-6-yl)-2-morpholinopyrimidin-4-amine: ¹H NMR (400 MHz, DMSO-d₆) δ: 9.55 (s, 1H), 8.27 (s, 1H), 8.10 (s, 1H), 7.83 (d, J=8.5 Hz, 1H), 7.78-7.67 (m, 3H), 7.41-7.32 (m, 2H), 6.68 (s, 1H), 4.57 (t, J=6.4 Hz, 2H), 3.82-3.73 (m, 8H), 2.73 (t, J=6.4 Hz, 2H), 2.19 (s, 6H); HPLC purity: 98.51%; LCMS Calculated for C₂₅H₂₈ClN₇O: 477.20; Observed: 478.30 (M+1).

N-(4-chlorophenyl)-6-(2-(2-(dimethylamino)ethyl)-2H-indazol-6-yl)-2-morpholinopyrimidin-4-amine: ¹H NMR (400 MHz, DMSO-d₆) δ: 9.52 (s, 1H), 8.42 (s, 1H), 8.27 (s, 1H), 7.78 (d, J=8.8 Hz, 1H), 7.76-7.61 (m, 3H), 7.41-7.32 (m, 2H), 6.64 (s, 1H), 4.54 (t, J=6.3 Hz, 2H), 3.82-3.72 (m, 8H), 2.81 (t, J=6.3 Hz, 2H), 2.18 (s, 6H); HPLC purity: 97.45%; LCMS Calculated for C₂₅H₂₈ClN₇O: 477.20; Observed: 478.25 (M+1).

N-(4-chlorophenyl)-6-(1-(3-(dimethylamino)propyl)-1H-indazol-6-yl)-2-morpholinopyrimidin-4-amine: ¹H NMR (400 MHz, DMSO-d₆) δ: 10.21 (d, J=10.2 Hz, 1H), 8.32 (s, 1H), 8.20 (s, 1H), 7.91 (d, J=8.4 Hz, 1H), 7.71 (dd, J=19.5, 8.4 Hz, 3H), 7.41 (d, J=8.3 Hz, 2H), 6.77 (s, 1H), 4.62 (q, J=8.3, 6.8 Hz, 3H), 3.86-3.74 (m, 8H), 3.19-3.06 (m, 2H), 2.74 (d, J=4.8 Hz, 6H), 2.28 (q, J=7.4, 6.8 Hz, 2H); HPLC purity: 97.90%; LCMS Calculated for C₂₆H₃₀ClN₇O: 491.22; Observed: 492.30 (M+1).

N-(4-chlorophenyl)-2-morpholino-6-(1-(2-(pyrrolidin-1-yl)ethyl)-1H-indazol-6-yl)pyrimidin-4-amine: ¹H NMR (400 MHz, Methanol-d4) δ: 8.27 (s, 1H), 8.06 (s, 1H), 7.81 (t, J=6.5 Hz, 2H), 7.66 (d, J=8.3 Hz, 2H), 7.33-7.26 (m, 2H), 6.62 (s, 1H), 4.64 (t, J=7.0 Hz, 2H), 3.88-3.80 (m, 8H), 3.05 (t, J=7.0 Hz, 2H), 2.60 (d, J=5.7 Hz, 4H), 1.83-1.75 (m, 4H); HPLC purity: 96.53%; LCMS Calculated for C₂₇H₃₀ClN₇O: 503.22; Observed: 504.30 (M+1).

N-(4-chlorophenyl)-2-morpholino-6-(1-(3-(pyrrolidin-1-yl)propyl)-1H-indazol-6-yl)pyrimidin-4-amine: ¹H NMR (400 MHz, Methanol-d4) δ: 8.25 (s, 1H), 8.05 (s, 1H), 7.80 (q, J=8.6 Hz, 2H), 7.66 (d, J=8.4 Hz, 2H), 7.30 (d, J=8.4 Hz, 2H), 6.61 (s, 1H), 4.56 (t, J=6.7 Hz, 2H), 3.88-3.80 (m, 8H), 2.57-2.45 (m, 6H), 2.18 (p, J=7.2 Hz, 2H), 1.82-1.74 (m, 4H); HPLC purity: 94.04%; LCMS Calculated for C₂₈H₃₂ClN₇O: 437.13; Observed: 438.25 (M+1).

N-(4-chlorophenyl)-2-morpholino-6-(1-(2-morpholinoethyl)-1H-indazol-6-yl)pyrimidin-4-amine: ¹H NMR (400 MHz, DMSO-d₆) δ: 11.48 (s, 1H), 10.10-10.01 (m, 1H), 8.44 (s, 1H), 8.25 (s, 1H), 7.93 (d, J=8.4 Hz, 1H), 7.74 (d, J=8.5 Hz, 3H), 7.40 (d, J=8.4 Hz, 2H), 6.76 (s, 1H), 5.03 (t, J=7.1 Hz, 2H), 3.98 (d, J=14.0 Hz, 2H), 3.88-3.64 (m, 12H), 3.51 (d, J=12.3 Hz, 2H), 3.18 (dd, J=15.6, 7.4 Hz, 2H); HPLC purity: 97%; LCMS Calculated for C₂₇H₃₀ClN₇O₂: 519.21; Observed: 520.30 (M+1).

N-(4-chlorophenyl)-2-morpholino-6-(1-(3-morpholinopropyl)-1H-indazol-6-yl)pyrimidin-4-amine: ¹H NMR (400 MHz, DMSO-d₆) δ: 10.03 (s, 1H), 8.30 (s, 1H), 8.20 (s, 1H), 7.91 (d, J=8.4 Hz, 1H), 7.72 (d, J=8.7 Hz, 3H), 7.41 (d, J=8.4 Hz, 2H), 6.74 (s, 1H), 4.82 (s, 1H), 4.61 (t, J=6.6 Hz, 2H), 3.99-3.91 (m, 2H), 3.85-3.62 (m, 8H), 3.44-3.37 (m, 4H), 3.19 (dd, J=9.9, 5.0 Hz, 2H), 3.07 (t, J=11.1 Hz, 2H), 2.32 (t, J=7.9 Hz, 2H); HPLC purity: 97.38%; LCMS Calculated for C₂₈H₃₂ClN₇O₂: 533.23; Observed: 534.40 (M+1).

N-(4-chlorophenyl)-6-(1-(2-methoxyethyl)-1H-indazol-6-yl)-2-morpholinopyrimidin-4-amine: ¹H NMR (400 MHz, DMSO-d₆) δ: 10.40 (s, 1H), 8.23 (s, 1H), 8.17 (s, 1H), 7.90 (d, J=8.4 Hz, 1H), 7.74 (d, J=8.4 Hz, 2H), 7.64 (d, J=8.5 Hz, 1H), 7.43 (d, J=8.4 Hz, 2H), 6.73 (s, 1H), 4.66 (t, J=5.4 Hz, 2H), 3.81-3.66 (m,10H), 3.21(s,3H); HPLC purity: 98.66%; LCMS Calculated for C₂₄H₂₅ClN₆O₂: 464.17; Observed: 465.25 (M+1).

N-(4-chlorophenyl)-2-morpholino-6-(1-(2-morpholinoethyl)-1H-indazol-5-yl)pyrimidin-4-amine: ¹H NMR (400 MHz, DMSO-d₆) δ: 9.50 (s, 1H), 8.44 (s, 1H), 8.19 (s, 1H), 8.01 (d, J=8.8 Hz, 1H), 7.77 (d, J=8.7 Hz, 1H), 7.71 (d, J=8.3 Hz, 2H), 7.36 (d, J=8.4 Hz, 2H), 6.63 (s, 1H), 4.59-4.51 (m, 2H), 3.78-3.72 (m, 8H), 3.52-3.45 (m, 4H), 2.78 (d, J=7.0 Hz, 2H), 2.42 (s, 4H); HPLC purity: 97.37%; LCMS Calculated for C₂₇H₃₀ClN₇O₂: 519.21; Observed: 520.35(M+1).

N-(4-chlorophenyl)-2-morpholino-6-(1-(3-morpholinopropyl)-1H-indazol-5-yl)pyrimidin-4-amine: ¹H NMR (400 MHz, DMSO-d₆) δ: 9.49 (s, 1H), 8.45 (d, J=1.8 Hz, 1H), 8.20 (s, 1H), 8.00 (dd, J=8.9, 1.6 Hz, 1H), 7.79-7.66 (m, 3H), 7.41-7.32 (m, 2H), 6.62 (s, 1H), 4.47 (t, J=6.6 Hz, 2H), 3.82-3.68 (m, 8H), 3.54 (t, J=4.6 Hz, 4H), 2.22 (dt, J=28.1, 5.8 Hz, 6H), 2.00 (p, J=6.8 Hz, 2H); HPLC purity: 99.06%; LCMS Calculated for C₂₈H₃₂ClN₇O₂: 533.23; Observed: 534.40 (M+1).

N-(4-chlorophenyl)-6-(1-cyclopropyl-3-methyl-1H-indazol-6-yl)-2-morpholinopyrimidin-4-amine: ¹H NMR (400 MHz, Methanol-d₄) δ: 8.07 (d, J=1.3 Hz, 1H), 7.92 (d, J=8.3 Hz, 1H), 7.66 (d, J=8.6 Hz, 2H), 7.54-7.41 (m, 3H), 6.61 (d, J=5.1 Hz, 1H), 3.92-3.81 (m, 8H), 3.73-3.63 (m, 1H), 2.58 (s, 3H), 1.26-1.17 (m, 4H); HPLC purity: 95.02%; LCMS Calculated for C₂₅H₂₅ClN₆O: 460.18; Observed: 461.30 (M+1).

N-(4-chlorophenyl)-6-(1-(cyclopropylmethyl)-3-methyl-1H-indazol-6-yl)-2-morpholinopyrimidin-4-amine: ¹H NMR (400 MHz, Methanol-d4) δ: 8.04 (s, 1H), 7.93 (d, J=8.0 Hz, 1H), 7.66-7.62(m,2H), 7.50-7.42 (m,3H), 6.56 (s, 1H), 4.33 (d, J=6.4 Hz, 2H),3.90-3.80 (m, 8H), 2.54 (s, 3H), 1.36-1.29 (m, 1H),0.58-0.46 (m, 4H); HPLC purity: 99.6%; LCMS Calculated for C₂₆H₂₇ClN₆O: 474.19; Observed: 475.35 (M+1).

N-(4-chlorophenyl)-6-(1-cyclobutyl-3-methyl-1H-indazol-6-yl)-2-morpholinopyrimidin-4-amine: ¹H NMR (400 MHz, DMSO-d₆) δ: 9.54 (s, 1H), 8.19 (s, 1H), 7.81-7.66 (m, 4H), 7.37 (d, J=8.5 Hz, 2H), 6.68 (s, 1H), 5.30 (p, J=8.3 Hz, 1H),3.80-3.70 (m, 8H), 2.72-2.57 (m, 2H), 2.54 (s, 3H), 2.49-2.39 (m, 2H), 1.87 (tq, J=6.9, 4.0, 2.4 Hz, 2H); HPLC purity: 99.9%; LCMS Calculated for C₂₆H₂₇ClN₆O: 474.19; Observed: 475.35 (M+1).

N-(4-chlorophenyl)-6-(1-(2-(dimethylamino)ethyl)-3-methyl4H-indazol-6-yl)-2-morpholinopyrimidin-4-amine: ¹H NMR (400 MHz, Methanol-d₄) δ: 8.19 (s, 1H), 7.78-7.63 (m, 4H), 7.34-7.26 (m, 2H), 6.62 (s, 1H), 4.59-4.51 (m, 2H), 3.89-3.78 (m, 8H), 2.89 (t, J=6.9 Hz, 2H), 2.57 (s, 3H), 2.34 (s, 6H); HPLC purity: 97.13%; LCMS Calculated for C₂₆H₃₀ClN₇O: 491.22; Observed: 492.40 (M+1).

N-(4-chlorophenyl)-6-(1-(3-(dimethylamino)propyl)-3-methyl-1H-indazol-6-yl)-2-morpholinopyrimidin-4-amine: ¹H NMR (400 MHz, Methanol-d₄) δ: 8.19 (s, 1H), 7.77 (q, J=8.7 Hz, 2H), 7.66 (d, J=8.5 Hz, 2H), 7.31 (d, J=8.3 Hz, 2H), 6.61 (s, 1H), 4.54 (q, J=6.4 Hz, 2H), 3.89-3.77 (m, 8H), 3.03 (t, J=7.9 Hz, 2H), 2.76 (s,6H), 2.59 (s, 3H), 2.30 (q, J=7.4 Hz, 2H); HPLC purity: 99.55%; LCMS Calculated for C₂₇H₃₂ClN₇O: 505.24; Observed: 506.40 (M+1).

N-(4-chlorophenyl)-6-(3-methyl-1-(2-(pyrrolidin-1-yl)ethyl)-1H-indazol-6-yl)-2-morpholinopyrimidin-4-amine: ¹H NMR (400 MHz, Methanol-d₄) δ: 8.28 (s,1H),7.96 (d, J=7.6 Hz, 1H), 7.64-7.44 (m, 5H),6.65 (s,1H), 4.87-4.81 (m, 2H), 3.92-3.86 (m, 10H), 3.73-3.63 (m, 2H), 3.18-3.10 (m, 2H), 2.63(s, 3H), 2.14 (d, J=8.2 Hz, 2H), 2.06-1.98 (m, 2H); HPLC purity: 91.62%; LCMS Calculated for C₂₈H₃₂ClN₇O: 517.24; Observed: 518.40 (M+1).

N-(4-chlorophenyl)-6-(3-methyl-1-(3-(pyrrolidin-1-yl)propyl)-1H-indazol-6-yl)-2-morpholinopyrimidin-4-amine: ¹H NMR (400 MHz, DMSO-d₆) δ: 9.55 (s, 1H), 8.18 (s, 1H), 7.81-7.66 (m, 4H), 7.37 (d, J=8.4 Hz, 2H), 6.68 (s, 1H), 4.43 (t, J=6.7 Hz, 2H), 3.82-3.72 (m, 8H),2.50 (s,3H),2.33 (dt, J=22.9, 6.1 Hz, 6H), 1.98 (dd, J=9.2, 4.5 Hz, 2H), 1.66 (q, J=4.2, 3.3 Hz, 4H); HPLC purity: 99.38%; LCMS Calculated for C₂₉H₃₄ClN₇O: 531.25; Observed: 532.40 (M+1).

N-(4-chlorophenyl)-6-(3-methyl-1-(2-morpholinoethyl)-1H-indazol-6-yl)-2-morpholinopyrimidin-4-amine: ¹H NMR (400 MHz, Methanol-d₄) δ: 8.18 (s, 1H), 7.74 (s, 2H), 7.66 (d, J=8.2 Hz, 2H), 7.33-7.26 (m, 2H), 6.61 (s, 1H), 4.54 (t, J=6.7 Hz, 2H), 3.87-3.76 (m,8H), 3.59 (dd, J=5.5, 3.1 Hz, 4H), 2.85 (t, J=6.6 Hz, 2H), 2.59-2.48 (m, 7H); HPLC purity: 97.74%; LCMS Calculated for C₂₈H₃₂ClN₇O₂: 533.23; Observed: 534.45 (M+1).

N-(4-chlorophenyl)-6-(3-methyl-1-(3-morpholinopropyl)-1H-indazol-6-yl)-2-morpholinopyrimidin-4-amine: ¹H NMR (400 MHz, D₂O) δ: 7.84-7.72 (m, 2H), 7.43-7.31 (m, 5H), 6.37 (s, 1H), 4.48-4.38 (m, 2H), 4.09 (d, J=11.6Hz, 2H),3.81-3.74(m,10H), 3.47 (d, J=11.2Hz, 2H),3.18-3.04 (m,4H), 2.46 (s, 3H),2.33-2.30 (m,2H); HPLC purity: 95.26%; LCMS Calculated for C₂₉H₃₄ClN₇O₂: 547.25; Observed: 548.30 (M+1).

N-(4-chlorophenyl)-6-(1,3-dimethyl-1H-indazol-6-yl)-2-morpholinopyrimidin-4-amine: ¹H NMR (400 MHz, Methanol-d4) δ: 8.04-7.98 (m, 1H), 7.92 (d, J=8.5 Hz, 1H), 7.65 (d, J=8.3 Hz, 2H), 7.52-7.41 (m, 3H), 6.58 (s, 1H), 4.10 (s, 3H), 3.93-3.81 (m, 8H), 2.60 (s, 3H); HPLC purity: 99.87%; LCMS Calculated for C₂₃H₂₃ClN₆O: 434.16; Observed: 435.20 (M+1).

### Examples 313-346

### Synthesis of 5 & 6 Substituted Benzimidazole & Indazoles Phenyl Carboxamides with Solubilizing Groups at the N1 Position

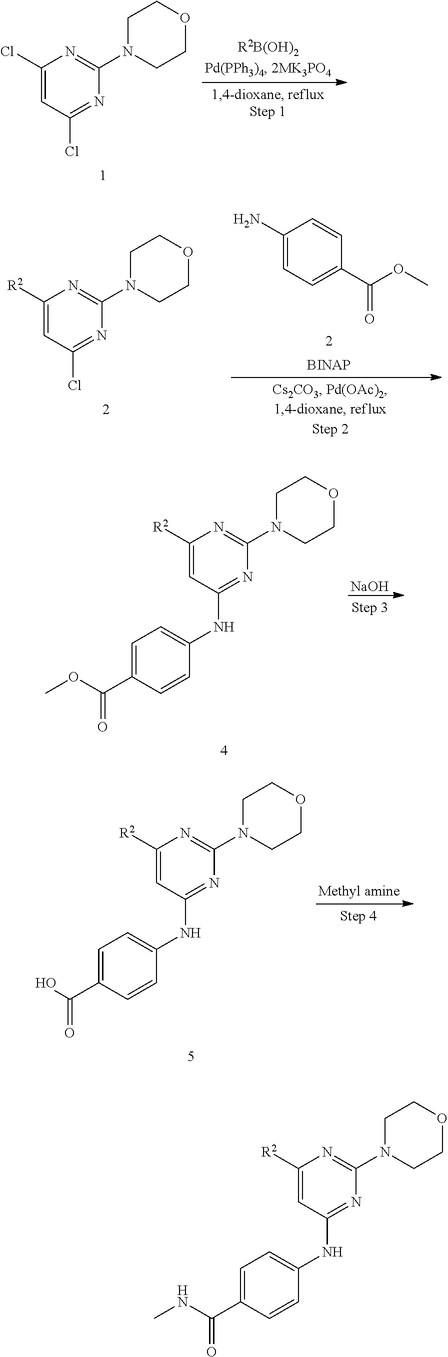

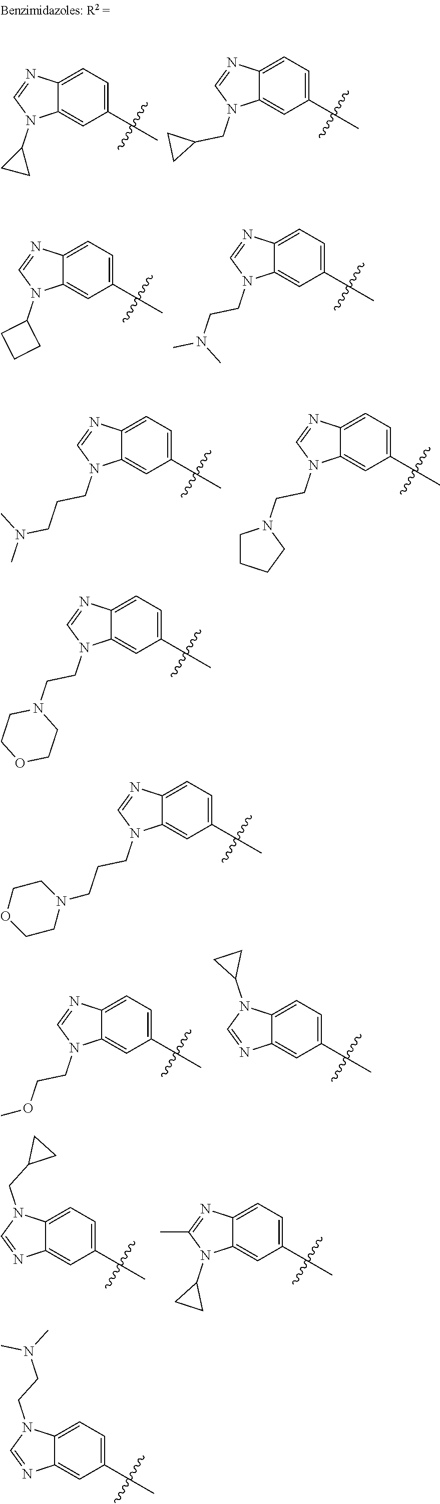

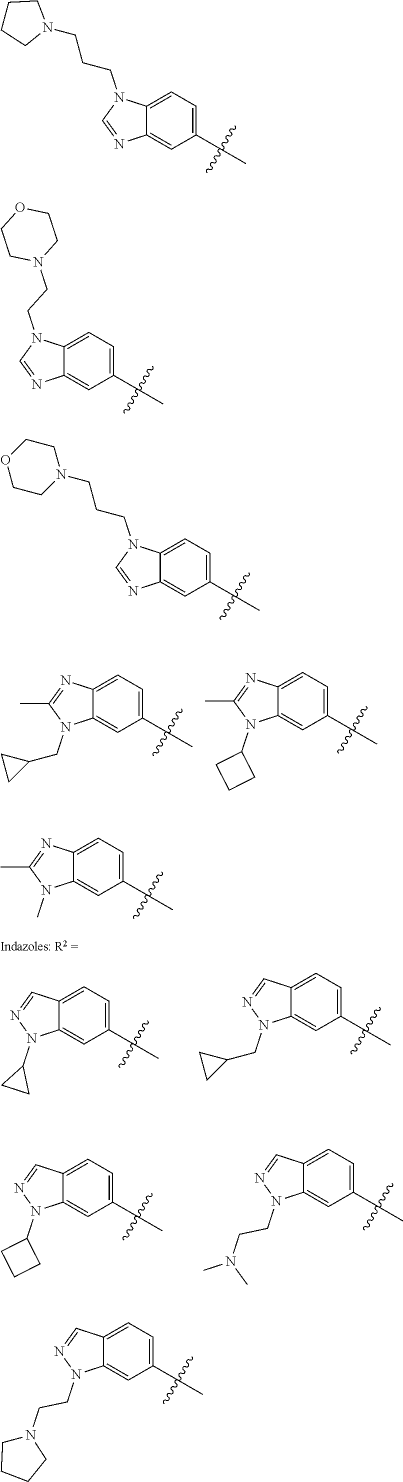

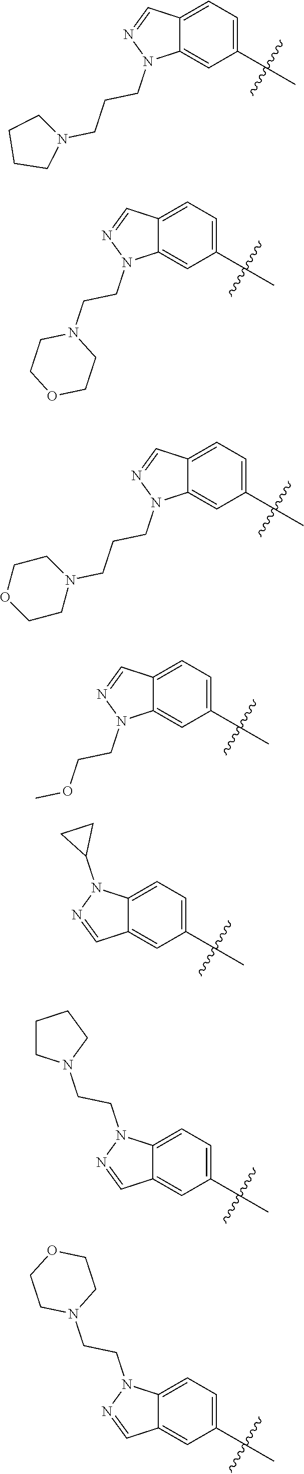

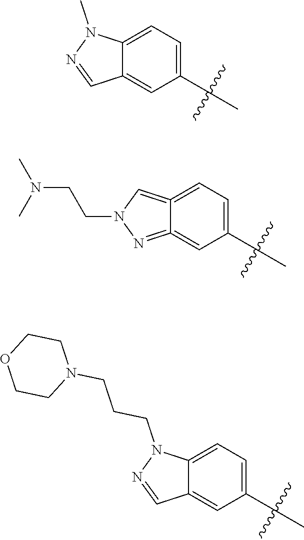

### Step 1: General Procedure for Suzuki Coupling (2)

The following intermediates were prepared using the general procedure for Suzuki coupling using the dichloro compound 1 and the respective boronic acid/boronate ester.

| Structure (Benzimidazoles) | LCMS |
|---|---|
| 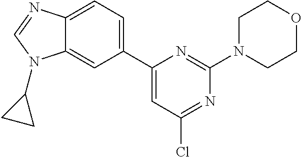 [SMILES: Clc1cc(-c2ccc3ncn(C4CC4)c3c2)nc(N2CCOCC2)n1] | LCMS (m/z): 356.10 (M + 1) |
| 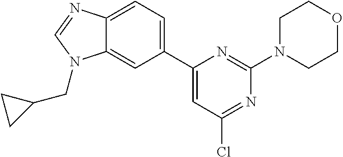 [SMILES: Clc1cc(-c2ccc3ncn(CC4CC4)c3c2)nc(N2CCOCC2)n1] | LCMS (m/z): 370.10 (M + 1) |
| 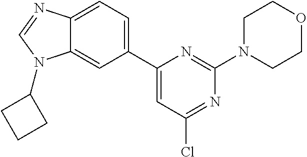 [SMILES: Clc1cc(-c2ccc3ncn(C4CCC4)c3c2)nc(N2CCOCC2)n1] | LCMS (m/z): 370.15 (M + 1) |
| 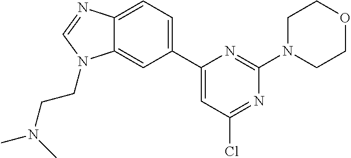 [SMILES: CN(C)CCn1cnc2ccc(-c3cc(Cl)nc(N4CCOCC4)n3)cc21] | LCMS (m/z): 387.25 (M + 1) |
| 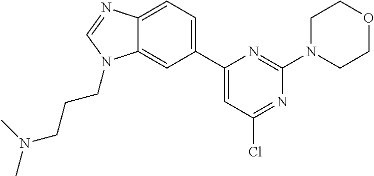 [SMILES: CN(C)CCCn1cnc2ccc(-c3cc(Cl)nc(N4CCOCC4)n3)cc21] | LCMS (m/z): 401.30 (M + 1) |
| 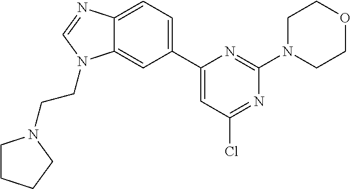 [SMILES: Clc1cc(-c2ccc3ncn(CCN4CCCC4)c3c2)nc(N2CCOCC2)n1] | LCMS (m/z): 413.25 (M + 1) |
| 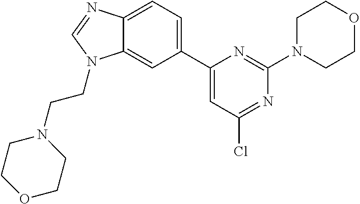 [SMILES: Clc1cc(-c2ccc3ncn(CCN4CCOCC4)c3c2)nc(N2CCOCC2)n1] | LCMS (m/z): 429.25 (M + 1) |
| 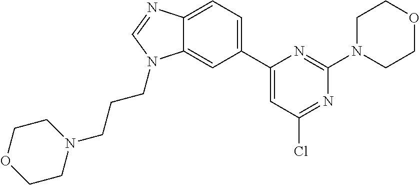 [SMILES: Clc1cc(-c2ccc3ncn(CCCN4CCOCC4)c3c2)nc(N2CCOCC2)n1] | LCMS (m/z): 443.35 (M + 1) |
| 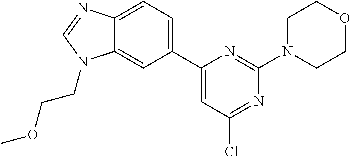 [SMILES: COCCn1cnc2ccc(-c3cc(Cl)nc(N4CCOCC4)n3)cc21] | LCMS (m/z): 374.15 (M + 1) |
| 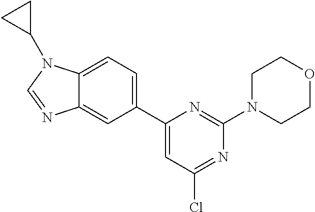 [SMILES: Clc1cc(-c2ccc3c(c2)ncn3C2CC2)nc(N2CCOCC2)n1] | LCMS (m/z): 356.05 (M + 1) |
| 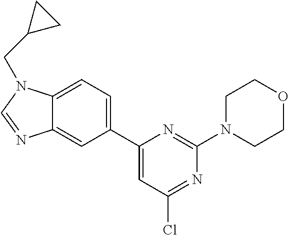 [SMILES: Clc1cc(-c2ccc3c(c2)ncn3CC2CC2)nc(N2CCOCC2)n1] | LCMS (m/z): 370.05 (M + 1) |
| 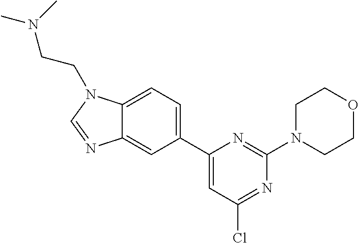 [SMILES: CN(C)CCn1cnc2cc(-c3cc(Cl)nc(N4CCOCC4)n3)ccc21] | LCMS (m/z): 387.30 (M + 1) |
| 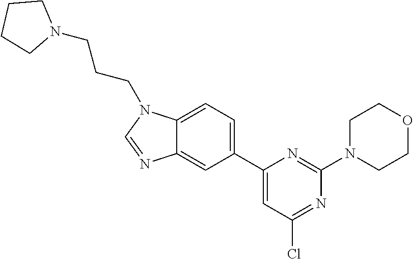 [SMILES: Clc1cc(-c2ccc3c(c2)ncn3CCCN2CCCC2)nc(N2CCOCC2)n1] | LCMS (m/z): 427.30 (M + 1) |
| 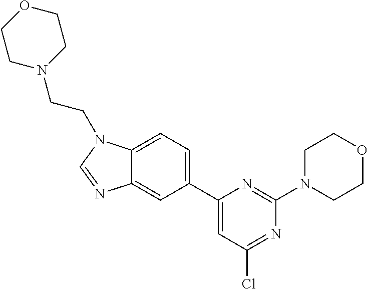 [SMILES: Clc1cc(-c2ccc3c(c2)ncn3CCN2CCOCC2)nc(N2CCOCC2)n1] | LCMS (m/z): 429.35 (M + 1) |
| 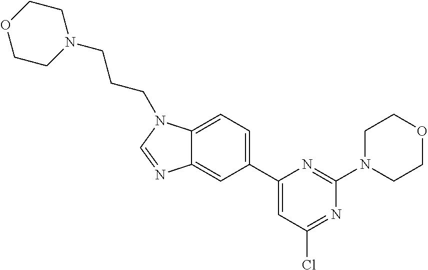 [SMILES: Clc1cc(-c2ccc3c(c2)ncn3CCCN2CCOCC2)nc(N2CCOCC2)n1] | LCMS (m/z): 443.35 (M + 1) |
| 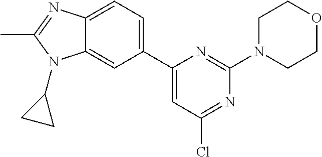 | LCMS (m/z): 384.20 (M + 1) |
| 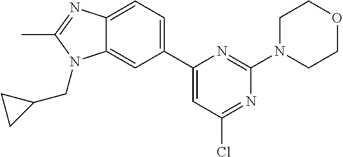 [SMILES: Cc1nc2ccc(-c3cc(Cl)nc(N4CCOCC4)n3)cc2n1CC1CC1] | LCMS (m/z): 384.20 (M + 1) |
| 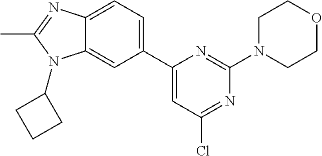 [SMILES: Cc1nc2ccc(-c3cc(Cl)nc(N4CCOCC4)n3)cc2n1C1CCC1] | LCMS (m/z): 384.30 (M + 1) |
| 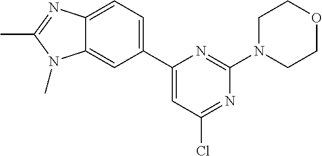 [SMILES: Cc1nc2ccc(-c3cc(Cl)nc(N4CCOCC4)n3)cc2n1C] | LCMS (m/z): 344.10 (M + 1) |
| 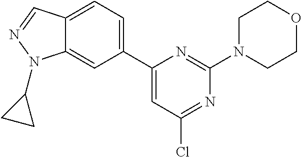 [SMILES: Clc1cc(-c2ccc3cnn(C4CC4)c3c2)nc(N2CCOCC2)n1] | LCMS (m/z): 356.15 (M + 1). |
| 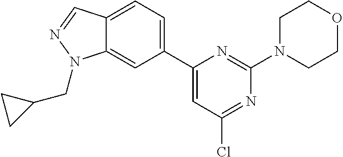 [SMILES: Clc1cc(-c2ccc3cnn(CC4CC4)c3c2)nc(N2CCOCC2)n1] | LCMS (m/z): 370.25 (M + 1) |
| 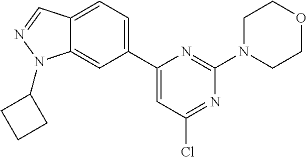 [SMILES: Clc1cc(-c2ccc3cnn(C4CCC4)c3c2)nc(N2CCOCC2)n1] | LCMS (m/z): 370.15 (M + 1) |
| 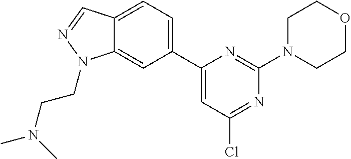 [SMILES: CN(C)CCn1ncc2ccc(-c3cc(Cl)nc(N4CCOCC4)n3)cc21] | LCMS (m/z): 387.20 (M + 1) |
| 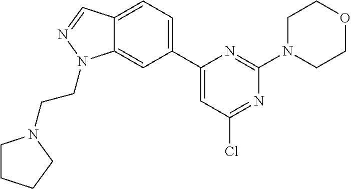 [SMILES: Clc1cc(-c2ccc3cnn(CCN4CCCC4)c3c2)nc(N2CCOCC2)n1] | LCMS (m/z): 413.30 (M + 1) |
| 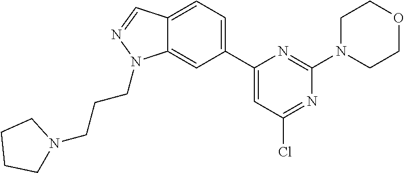 [SMILES: Clc1cc(-c2ccc3cnn(CCCN4CCCC4)c3c2)nc(N2CCOCC2)n1] | LCMS (m/z): 427.25 (M + 1) |
| 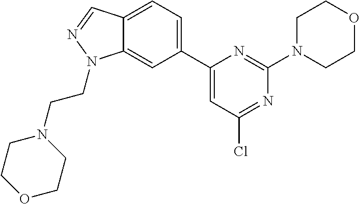 [SMILES: Clc1cc(-c2ccc3cnn(CCN4CCOCC4)c3c2)nc(N2CCOCC2)n1] | LCMS (m/z): 429.25 (M + 1) |
| 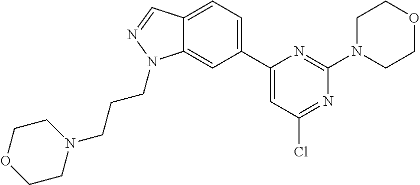 [SMILES: Clc1cc(-c2ccc3cnn(CCCN4CCOCC4)c3c2)nc(N2CCOCC2)n1] | LCMS (m/z): 443.25 (M + 1) |
| 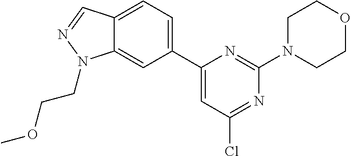 [SMILES: COCCn1ncc2ccc(-c3cc(Cl)nc(N4CCOCC4)n3)cc21] | LCMS (m/z): 374.20 (M + 1) |
| 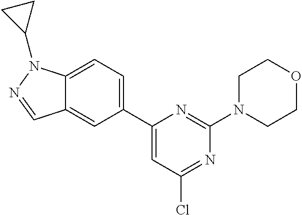 [SMILES: Clc1cc(-c2ccc3c(cnn3C3CC3)c2)nc(N2CCOCC2)n1] | LCMS (m/z): 356.15 (M + 1) |
| 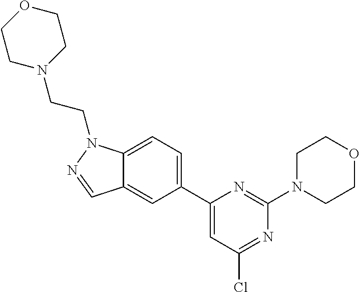 [SMILES: Clc1cc(-c2ccc3c(cnn3CCN3CCOCC3)c2)nc(N2CCOCC2)n1] | LCMS (m/z): 429.20 (M + 1) |
| 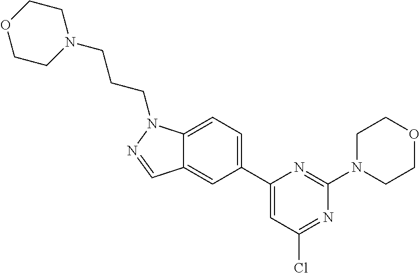 [SMILES: Clc1cc(-c2ccc3c(cnn3CCCN3CCOCC3)c2)nc(N2CCOCC2)n1] | — |
| 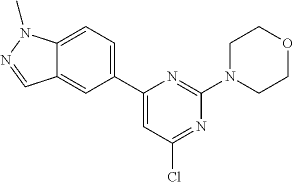 [SMILES: Cn1ncc2cc(-c3cc(Cl)nc(N4CCOCC4)n3)ccc21] | LCMS (m/z): 330.05 (M + 1) |

### Step 2: General Procedure for Buchwald Coupling (4)

The following compounds were prepared using the general procedure as described above for Buchwald coupling using the respective chloro compound 2 and methyl-4-amino benzoate 3 to provide compound 4.

| Structure | LCMS |
|---|---|
| 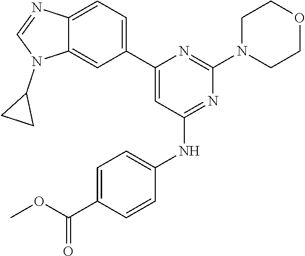 [SMILES: COC(=O)c1ccc(Nc2cc(-c3ccc4ncn(C5CC5)c4c3)nc(N3CCOCC3)n2)cc1] | LCMS (m/z): 471.00 (M + 1) |
| 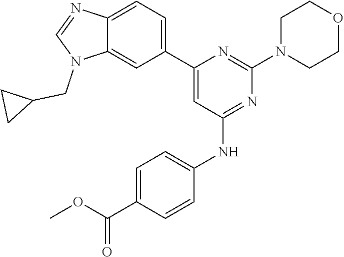 [SMILES: COC(=O)c1ccc(Nc2cc(-c3ccc4ncn(CC5CC5)c4c3)nc(N3CCOCC3)n2)cc1] | LCMS (m/z): 485.25 (M + 1) |
| 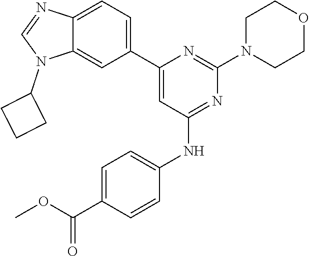 [SMILES: COC(=O)c1ccc(Nc2cc(-c3ccc4ncn(C5CCC5)c4c3)nc(N3CCOCC3)n2)cc1] | LCMS (m/z): 485.40 (M + 1) |
| 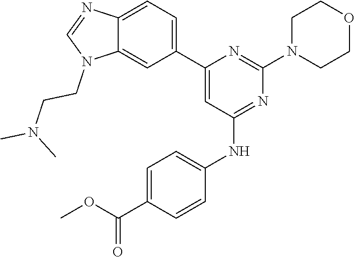 [SMILES: COC(=O)c1ccc(Nc2cc(-c3ccc4ncn(CCN(C)C)c4c3)nc(N3CCOCC3)n2)cc1] | LCMS (m/z): 502.40 (M + 1) |
| 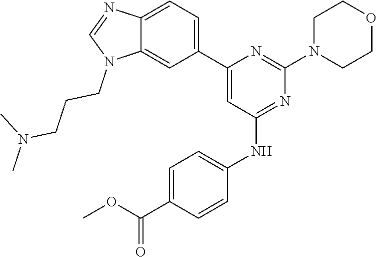 [SMILES: COC(=O)c1ccc(Nc2cc(-c3ccc4ncn(CCCN(C)C)c4c3)nc(N3CCOCC3)n2)cc1] | LCMS (m/z): 516.45 (M + 1) |
| 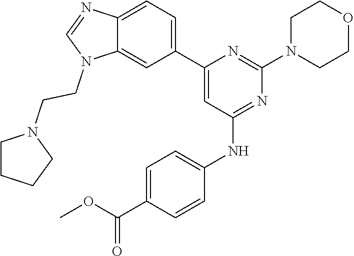 [SMILES: COC(=O)c1ccc(Nc2cc(-c3ccc4ncn(CCN5CCCC5)c4c3)nc(N3CCOCC3)n2)cc1] | LCMS (m/z): 528.05 (M + 1) |
| 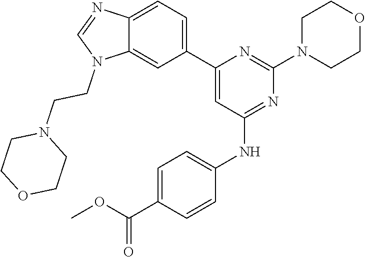 [SMILES: COC(=O)c1ccc(Nc2cc(-c3ccc4ncn(CCN5CCOCC5)c4c3)nc(N3CCOCC3)n2)cc1] | LCMS (m/z): 544.45 (M + 1) |
| 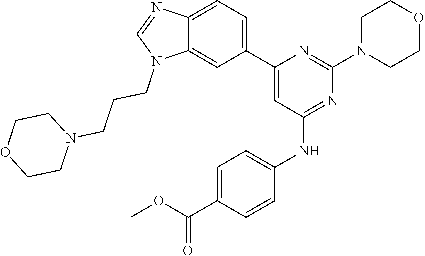 [SMILES: COC(=O)c1ccc(Nc2cc(-c3ccc4ncn(CCCN5CCOCC5)c4c3)nc(N3CCOCC3)n2)cc1] | LCMS (m/z): 558.50 (M + 1) |
| 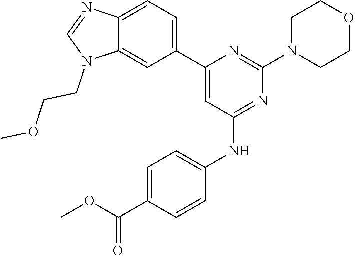 [SMILES: COCCn1cnc2ccc(-c3cc(Nc4ccc(C(=O)OC)cc4)nc(N4CCOCC4)n3)cc21] | LCMS (m/z): 489.30 (M + 1) |
| 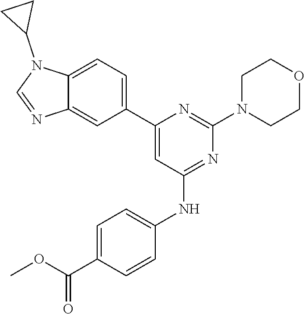 [SMILES: COC(=O)c1ccc(Nc2cc(-c3ccc4c(c3)ncn4C3CC3)nc(N3CCOCC3)n2)cc1] | LCMS (m/z): 471.25 (M + 1) |
| 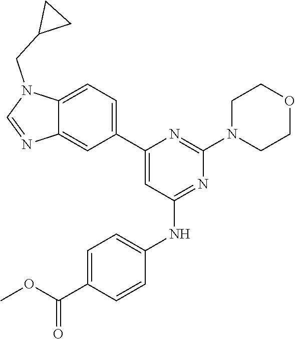 [SMILES: COC(=O)c1ccc(Nc2cc(-c3ccc4c(c3)ncn4CC3CC3)nc(N3CCOCC3)n2)cc1] | LCMS (m/z): 485.35 (M + 1) |
| 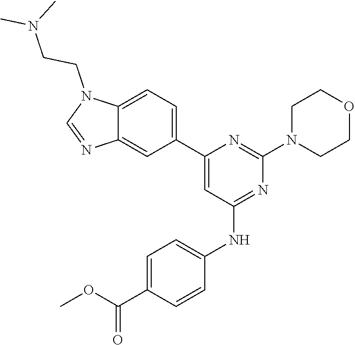 [SMILES: COC(=O)c1ccc(Nc2cc(-c3ccc4c(c3)ncn4CCN(C)C)nc(N3CCOCC3)n2)cc1] | LCMS (m/z): 502.45 (M + 1) |
| 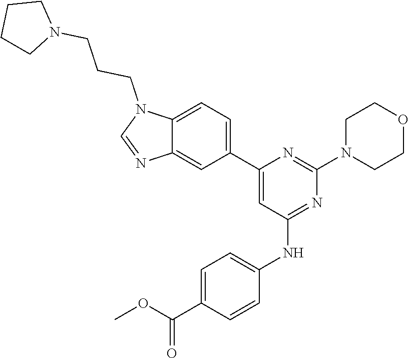 [SMILES: COC(=O)c1ccc(Nc2cc(-c3ccc4c(c3)ncn4CCCN3CCCC3)nc(N3CCOCC3)n2)cc1] | LCMS (m/z): 542.45 (M + 1) |
| 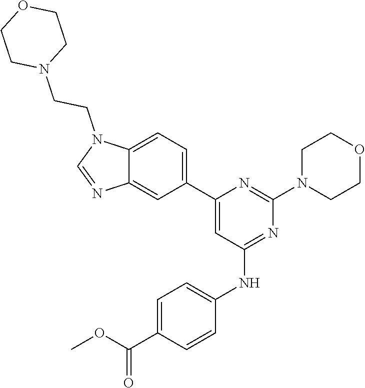 [SMILES: COC(=O)c1ccc(Nc2cc(-c3ccc4c(c3)ncn4CCN3CCOCC3)nc(N3CCOCC3)n2)cc1] | LCMS (m/z): 544.55 (M + 1) |
| 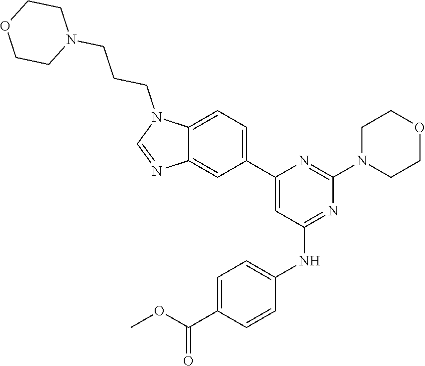 [SMILES: COC(=O)c1ccc(Nc2cc(-c3ccc4c(c3)ncn4CCCN3CCOCC3)nc(N3CCOCC3)n2)cc1] | LCMS (m/z): 558.55 (M + 1) |
| 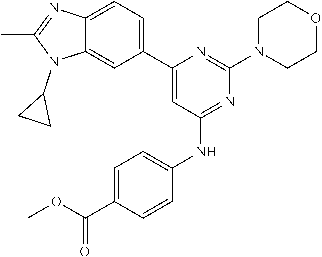 [SMILES: COC(=O)c1ccc(Nc2cc(-c3ccc4nc(C)n(C5CC5)c4c3)nc(N3CCOCC3)n2)cc1] | LCMS (m/z): 485.40 (M + 1) |
| 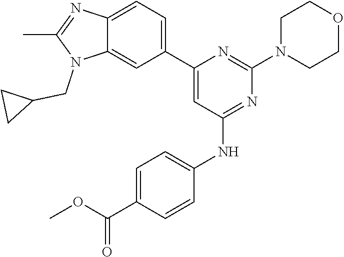 [SMILES: COC(=O)c1ccc(Nc2cc(-c3ccc4nc(C)n(CC5CC5)c4c3)nc(N3CCOCC3)n2)cc1] | LCMS (m/z): 499.40 (M + 1) |
| 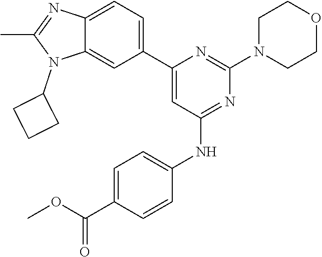 | LCMS (m/z): 599.40 (M + 1) |
| 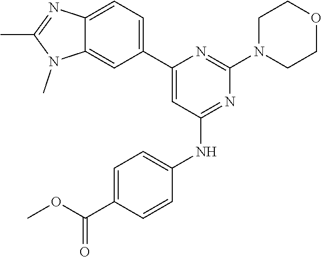 [SMILES: COC(=O)c1ccc(Nc2cc(-c3ccc4nc(C)n(C)c4c3)nc(N3CCOCC3)n2)cc1] | LCMS (m/z): 459.35 (M + 1) |

| Structure (Indazoles) | LCMS |
|---|---|
| 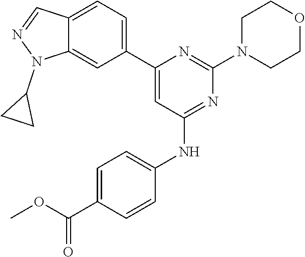 [SMILES: COC(=O)c1ccc(Nc2cc(-c3ccc4cnn(C5CC5)c4c3)nc(N3CCOCC3)n2)cc1] | LCMS (m/z): 471.25 (M + 1) |
| 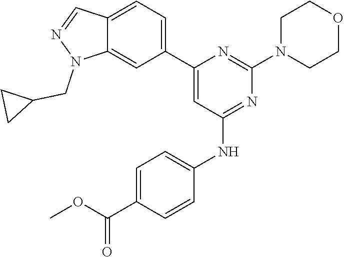 [SMILES: COC(=O)c1ccc(Nc2cc(-c3ccc4cnn(CC5CC5)c4c3)nc(N3CCOCC3)n2)cc1] | LCMS (m/z): 485.30(M + 1) |
| 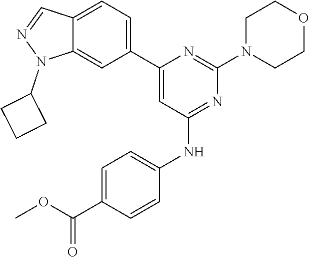 [SMILES: COC(=O)c1ccc(Nc2cc(-c3ccc4cnn(C5CCC5)c4c3)nc(N3CCOCC3)n2)cc1] | LCMS (m/z): 485.30 (M + 1) |
| 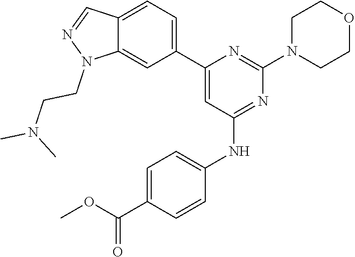 [SMILES: COC(=O)c1ccc(Nc2cc(-c3ccc4cnn(CCN(C)C)c4c3)nc(N3CCOCC3)n2)cc1] | LCMS (m/z): 502.35 (M + 1) |
| 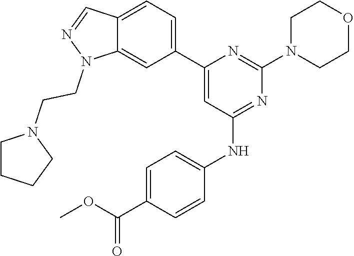 [SMILES: COC(=O)c1ccc(Nc2cc(-c3ccc4cnn(CCN5CCCC5)c4c3)nc(N3CCOCC3)n2)cc1] | monitored by TLC |
| 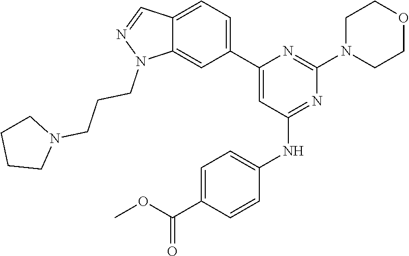 [SMILES: COC(=O)c1ccc(Nc2cc(-c3ccc4cnn(CCCN5CCCC5)c4c3)nc(N3CCOCC3)n2)cc1] | LCMS (m/z): 542.35 (M + 1) |
| 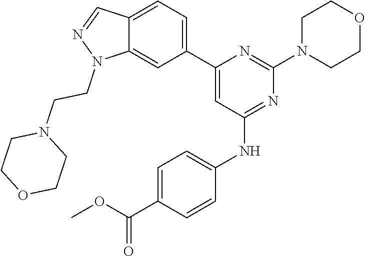 [SMILES: COC(=O)c1ccc(Nc2cc(-c3ccc4cnn(CCN5CCOCC5)c4c3)nc(N3CCOCC3)n2)cc1] | LCMS (m/z): 544.45 (M + 1) |
| 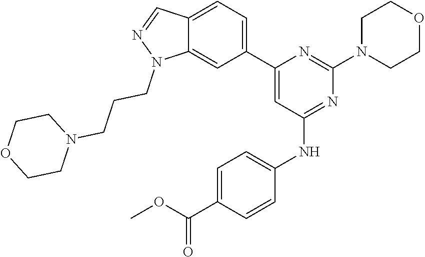 [SMILES: COC(=O)c1ccc(Nc2cc(-c3ccc4cnn(CCCN5CCOCC5)c4c3)nc(N3CCOCC3)n2)cc1] | LCMS (m/z): 558.40 (M + 1) |
| 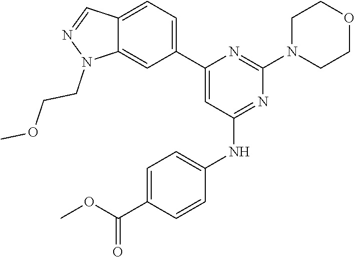 [SMILES: COCCn1ncc2ccc(-c3cc(Nc4ccc(C(=O)OC)cc4)nc(N4CCOCC4)n3)cc21] | LCMS (m/z): 489.30 (M + 1) |
| 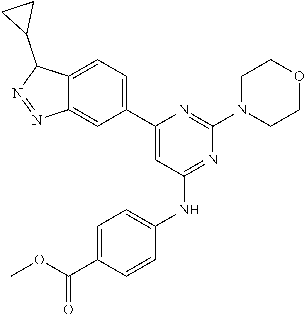 [SMILES: COC(=O)c1ccc(Nc2cc(-c3ccc4c(c3)N=NC4C3CC3)nc(N3CCOCC3)n2)cc1] | LCMS (m/z): 471.30 (M + 1) |
| 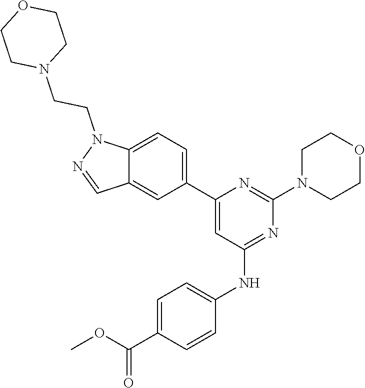 [SMILES: COC(=O)c1ccc(Nc2cc(-c3ccc4c(cnn4CCN4CCOCC4)c3)nc(N3CCOCC3)n2)cc1] | LCMS (m/z): 544.40 (M + 1) |
| 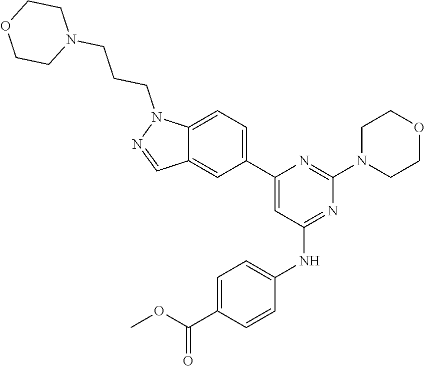 [SMILES: COC(=O)c1ccc(Nc2cc(-c3ccc4c(cnn4CCCN4CCOCC4)c3)nc(N3CCOCC3)n2)cc1] | LCMS (m/z): 557.00 (M + 1) |
| 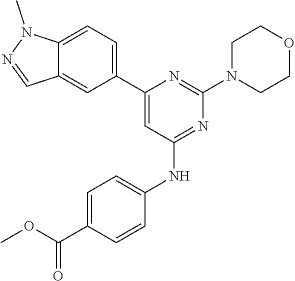 [SMILES: COC(=O)c1ccc(Nc2cc(-c3ccc4c(cnn4C)c3)nc(N3CCOCC3)n2)cc1] | LCMS (m/z): 445.30 (M + 1) |

### Step 3: General Procedure for Ester Hydrolysis (5)

The following compounds were prepared using the general procedure as described above for ester hydrolysis using the appropriate ester compound 3 and NaOH to provide compound 5. The crude acid 5 was used as such for the next step.

| Structure | LCMS |
|---|---|
| 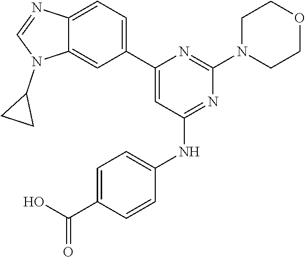 [SMILES: O=C(O)c1ccc(Nc2cc(-c3ccc4ncn(C5CC5)c4c3)nc(N3CCOCC3)n2)cc1] | LCMS (m/z): 457.15(M + 1) |
| 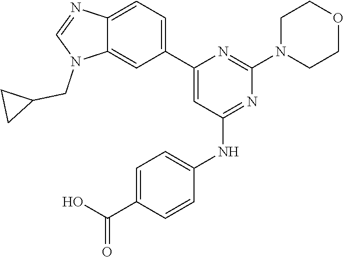 [SMILES: O=C(O)c1ccc(Nc2cc(-c3ccc4ncn(CC5CC5)c4c3)nc(N3CCOCC3)n2)cc1] | LCMS (m/z): 471.25(M + 1) |
| 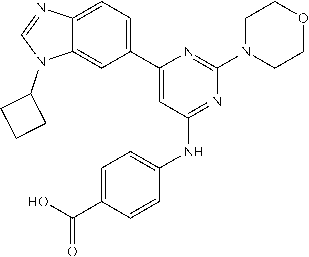 [SMILES: O=C(O)c1ccc(Nc2cc(-c3ccc4ncn(C5CCC5)c4c3)nc(N3CCOCC3)n2)cc1] | monitored by TLC |
| 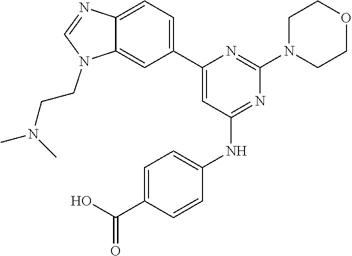 [SMILES: CN(C)CCn1cnc2ccc(-c3cc(Nc4ccc(C(=O)O)cc4)nc(N4CCOCC4)n3)cc21] | LCMS (m/z): 488.40(M + 1) |
| 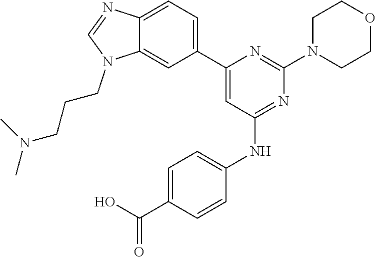 [SMILES: CN(C)CCCn1cnc2ccc(-c3cc(Nc4ccc(C(=O)O)cc4)nc(N4CCOCC4)n3)cc21] | LCMS (m/z): 502.45(M + 1) |
| 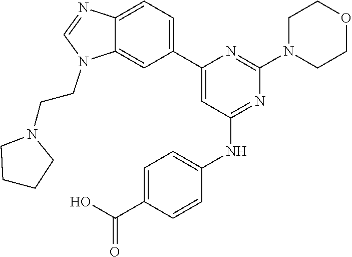 [SMILES: O=C(O)c1ccc(Nc2cc(-c3ccc4ncn(CCN5CCCC5)c4c3)nc(N3CCOCC3)n2)cc1] | LCMS (m/z): 514.45(M + 1) |
| 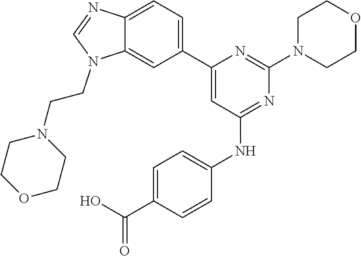 [SMILES: O=C(O)c1ccc(Nc2cc(-c3ccc4ncn(CCN5CCOCC5)c4c3)nc(N3CCOCC3)n2)cc1] | LCMS (m/z): 530.35(M + 1) |
| 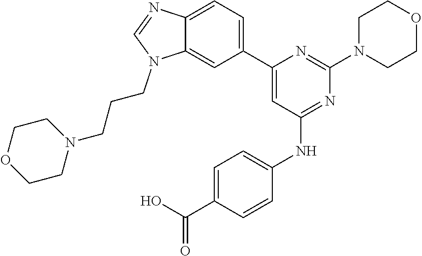 [SMILES: O=C(O)c1ccc(Nc2cc(-c3ccc4ncn(CCCN5CCOCC5)c4c3)nc(N3CCOCC3)n2)cc1] | LCMS (m/z): 544.45(M + 1) |
| 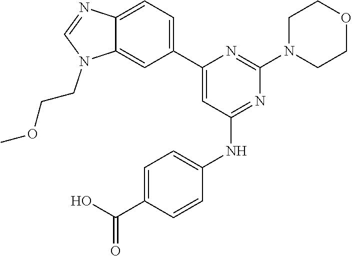 [SMILES: COCCn1cnc2ccc(-c3cc(Nc4ccc(C(=O)O)cc4)nc(N4CCOCC4)n3)cc21] | LCMS (m/z): 475.35(M + 1) |
| 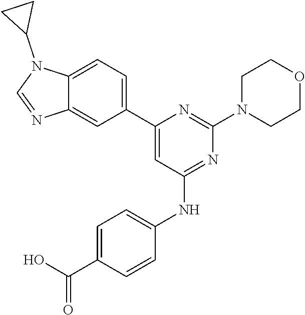 [SMILES: O=C(O)c1ccc(Nc2cc(-c3ccc4c(c3)ncn4C3CC3)nc(N3CCOCC3)n2)cc1] | LCMS (m/z): 457.50(M + 1) |
| 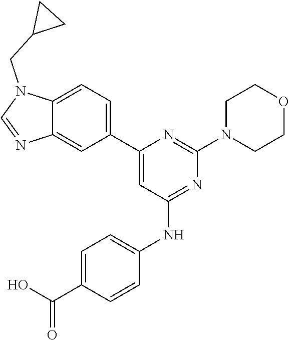 [SMILES: O=C(O)c1ccc(Nc2cc(-c3ccc4c(c3)ncn4CC3CC3)nc(N3CCOCC3)n2)cc1] | LCMS (m/z): 471.30 (M + 1) |
| 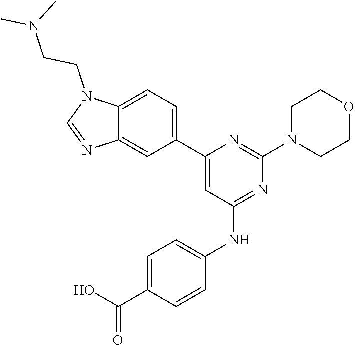 [SMILES: CN(C)CCn1cnc2cc(-c3cc(Nc4ccc(C(=O)O)cc4)nc(N4CCOCC4)n3)ccc21] | LCMS (m/z): 488.45 (M + 1) |
| 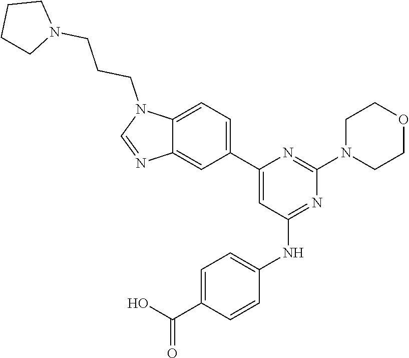 [SMILES: O=C(O)c1ccc(Nc2cc(-c3ccc4c(c3)ncn4CCCN3CCCC3)nc(N3CCOCC3)n2)cc1] | LCMS (m/z): 528.50(M + 1) |
| 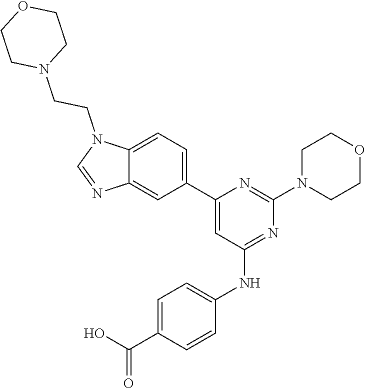 [SMILES: O=C(O)c1ccc(Nc2cc(-c3ccc4c(c3)ncn4CCN3CCOCC3)nc(N3CCOCC3)n2)cc1] | LCMS (m/z): 530.45 (M + 1) |
| 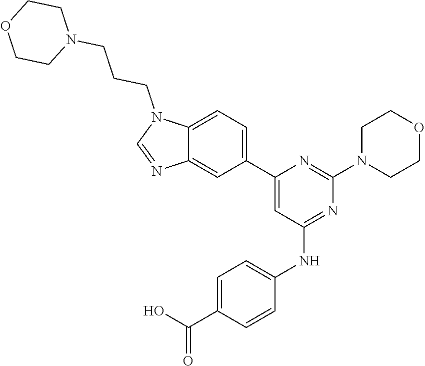 [SMILES: O=C(O)c1ccc(Nc2cc(-c3ccc4c(c3)ncn4CCCN3CCOCC3)nc(N3CCOCC3)n2)cc1] | LCMS (m/z): 544.45 (M + 1) |
| 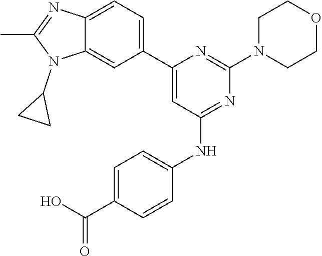 [SMILES: Cc1nc2ccc(-c3cc(Nc4ccc(C(=O)O)cc4)nc(N4CCOCC4)n3)cc2n1C1CC1] | LCMS (m/z): 471.30 (M + 1) |
| 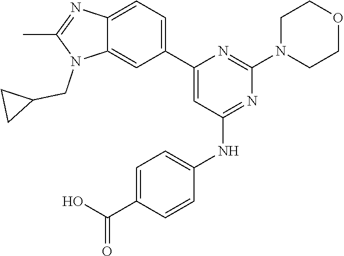 [SMILES: Cc1nc2ccc(-c3cc(Nc4ccc(C(=O)O)cc4)nc(N4CCOCC4)n3)cc2n1CC1CC1] | LCMS (m/z): 485.35 (M + 1) |
| 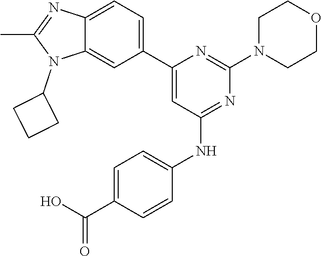 [SMILES: Cc1nc2ccc(-c3cc(Nc4ccc(C(=O)O)cc4)nc(N4CCOCC4)n3)cc2n1C1CCC1] | LCMS (m/z): 485.30 (M + 1). |
| 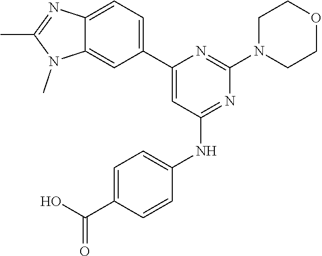 [SMILES: Cc1nc2ccc(-c3cc(Nc4ccc(C(=O)O)cc4)nc(N4CCOCC4)n3)cc2n1C] | LCMS (m/z): 445.25 (M + 1). |

| Structure (Indazoles) | LCMS |
|---|---|
| 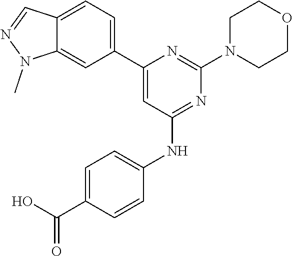 | LCMS (m/z): 457.25 (M + 1). |
| 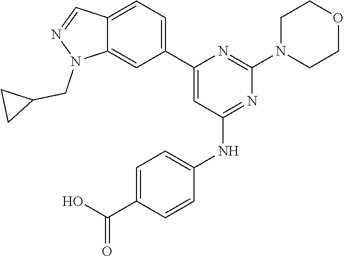 [SMILES: O=C(O)c1ccc(Nc2cc(-c3ccc4cnn(CC5CC5)c4c3)nc(N3CCOCC3)n2)cc1] | monitored by TLC |
| 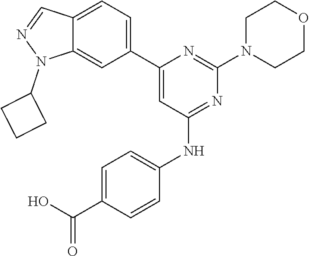 [SMILES: O=C(O)c1ccc(Nc2cc(-c3ccc4cnn(C5CCC5)c4c3)nc(N3CCOCC3)n2)cc1] | LCMS (m/z): 471.35(M + 1) |
| 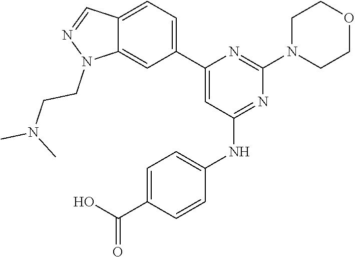 [SMILES: CN(C)CCn1ncc2ccc(-c3cc(Nc4ccc(C(=O)O)cc4)nc(N4CCOCC4)n3)cc21] | LCMS (m/z): 488.30(M + 1) |
| 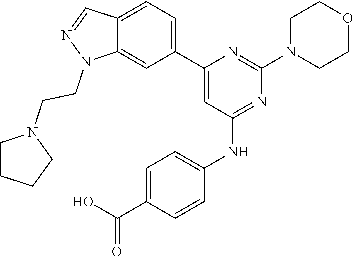 [SMILES: O=C(O)c1ccc(Nc2cc(-c3ccc4cnn(CCN5CCCC5)c4c3)nc(N3CCOCC3)n2)cc1] | LCMS (m/z): 514.40 (M + 1) |
| 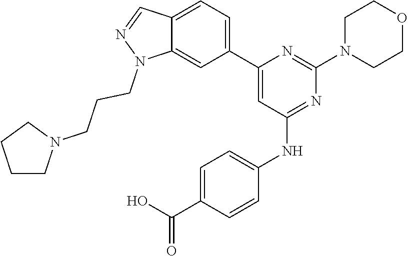 [SMILES: O=C(O)c1ccc(Nc2cc(-c3ccc4cnn(CCCN5CCCC5)c4c3)nc(N3CCOCC3)n2)cc1] | LCMS (m/z): 528.40 (M + 1) |
| 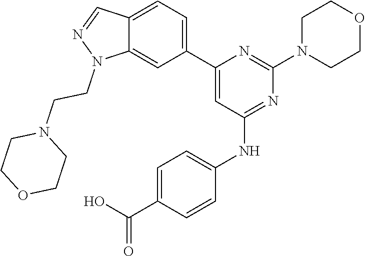 [SMILES: O=C(O)c1ccc(Nc2cc(-c3ccc4cnn(CCN5CCOCC5)c4c3)nc(N3CCOCC3)n2)cc1] | LCMS (m/z): 530.35 (M + 1) |
| 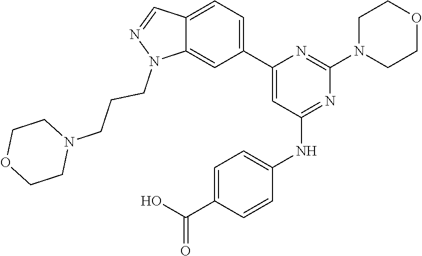 [SMILES: O=C(O)c1ccc(Nc2cc(-c3ccc4cnn(CCCN5CCOCC5)c4c3)nc(N3CCOCC3)n2)cc1] | LCMS (m/z): 544.45 (M + 1) |
| 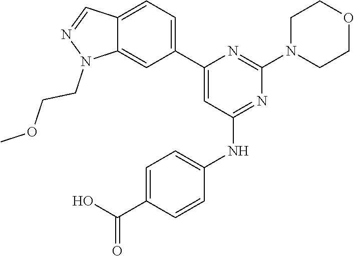 [SMILES: COCCn1ncc2ccc(-c3cc(Nc4ccc(C(=O)O)cc4)nc(N4CCOCC4)n3)cc21] | LCMS (m/z): 475.35 (M + 1) |
| 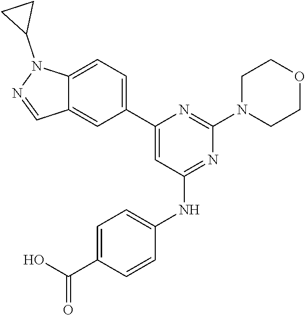 [SMILES: O=C(O)c1ccc(Nc2cc(-c3ccc4c(cnn4C4CC4)c3)nc(N3CCOCC3)n2)cc1] | LCMS (m/z): 457.30 (M + 1) |
| 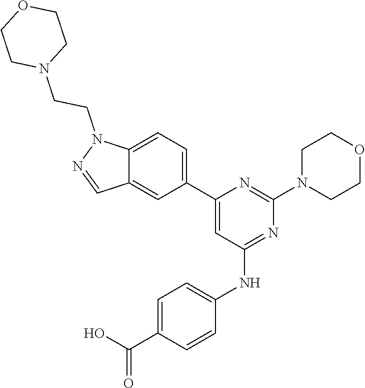 [SMILES: O=C(O)c1ccc(Nc2cc(-c3ccc4c(cnn4CCN4CCOCC4)c3)nc(N3CCOCC3)n2)cc1] | LCMS (m/z): 530.40 (M + 1) |
| 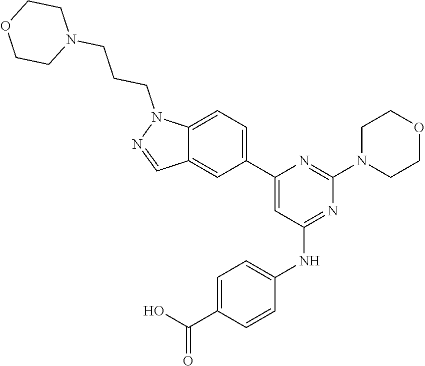 [SMILES: O=C(O)c1ccc(Nc2cc(-c3ccc4c(cnn4CCCN4CCOCC4)c3)nc(N3CCOCC3)n2)cc1] | monitored by TLC |
| 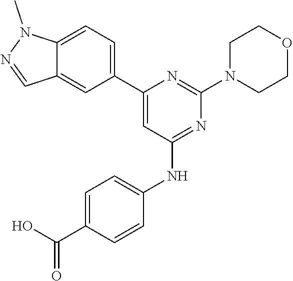 [SMILES: Cn1ncc2cc(-c3cc(Nc4ccc(C(=O)O)cc4)nc(N4CCOCC4)n3)ccc21] | LCMS (m/z): 431.35 (M + 1) |

### Step 4: General Procedure for Amide Coupling

The following compounds were prepared using the general procedure as described above for the peptide coupling using the appropriate acid 5 and methyl amine and PYBOP, DIPEA in DMF. The crude product was purified by preparative HPLC to afford the desired products.

4-((6-(1-cyclopropyl-1H-benzo[d]imidazol-6-yl)-2-morpholinopyrimidin-4-yl)amino)-N-methyl benzamide: ¹H NMR (400 MHz, DMSO-d₆) δ: 9.68 (s, 1H), 8.34-8.20 (m, 3H), 7.95 (dd, J=8.6, 1.7 Hz, 1H), 7.86-7.70 (m, 5H), 6.73 (s, 1H), 3.85-3.56 (m, 8H),3.64-3.58 (m,1H), 2.77 (d, J=4.4 Hz, 3H), 1.19-1.04 (m, 4H); HPLC purity: 95.17%; LCMS Calculated for C₂₆H₂₇N₇O₂: 469.22; observed: 470.30 (M+1).

4-((6-(1-(cyclopropylmethyl)-1H-benzo[d]imidazol-6-yl)-2-morpholinopyrimidin-4-yl)amino)-N-methylbenzamide: ¹H NMR (400 MHz, DMSO-d₆) δ: 9.65 (s, 1H), 8.37 (s, 1H), 8.27 (d, J=5.4 Hz, 2H), 7.91 (dd, J=8.4, 1.6 Hz, 1H), 7.86-7.70 (m, 5H), 6.73 (s, 1H), 4.21 (d, J=7.1 Hz, 2H), 3.86-3.74 (m, 8H), 2.77 (d, J=4.2 Hz, 3H), 1.33-1.29 (m, 1H), 0.64-0.54 (m, 2H), 0.52-0.43 (m, 2H); HPLC purity: 95.35%; LCMS Calculated for C₂₇H₂₉N₇O₂: 483.24; observed: 484.30 (M+1).

4-((6-(1-cyclobutyl-1H-benzo[d]imidazol-6-yl)-2-morpholinopyrimidin-4-yl)amino)-N-methylbenzamide: ¹H NMR (400 MHz, DMSO-d₆) δ: 9.50 (s, 1H), 8.35 (d, J=1.4 Hz, 1H), 8.18-8.10 (m, 1H), 7.92 (d, J=8.7 Hz, 1H), 7.75 (q, J=8.7 Hz, 5H), 5.21 (p, J=8.4 Hz, 1H), 3.78-3.67 (m, 8H), 2.76 (s, 3H), 2.69-2.60 (m, 4H), 1.97 (dt, J=21.8, 9.9 Hz, 2H); HPLC purity: 99.07%; LCMS Calculated for C₂₇H₂₉N₇O₂ (free base): 483.24; observed: 484.40 (M+1).

4-((6-(1-(2-(dimethylamino)ethyl)-1H-benzo[d]imidazol-6-yl)-2-morpholinopyrimidin-4-yl)amino)-N-methylbenzamide: ¹H NMR (400 MHz, CDCl₃) δ: 8.13 (s, 1H), 8.07 (s, 1H), 7.90-7.74 (m, 4H), 7.60-7.53 (m, 2H), 6.79 (s, 1H), 6.57 (s, 1H), 6.11 (q, J=4.6 Hz, 1H), 4.31 (t, J=6.6 Hz, 2H), 3.94-3.82 (m, 8H), 3.03 (d, J=4.8 Hz, 3H), 2.77 (t, J=6.5 Hz, 2H), 2.32 (s, 6H); HPLC purity: 96.66%; LCMS Calculated for C₂₇H₃₂N₈O₂: 500.26; observed: 501.45 (M+1).

4-((6-(1-(3-(dimethylamino)propyl)-1H-benzo[d]imidazol-6-yl)-2-morpholinopyrimidin-4-yl)amino)-N-methylbenzamide: ¹H NMR (400 MHz, CD₃OD) δ: 8.33-8.23 (m, 2H), 7.96 (dd, J=8.6, 1.7 Hz, 1H), 7.77 (d, J=16.2 Hz, 5H), 6.66 (s, 1H), 4.43 (t, J=6.9 Hz, 2H), 3.95-3.78 (m, 8H), 2.92 (s, 3H), 2.34 (q, J=6.7, 6.0 Hz, 2H), 2.24 (s, 6H), 2.13 (p, J=6.9 Hz, 2H); HPLC purity: 99.08%; LCMS Calculated for C₂₈H₃₄N₈O₂: 514.28; observed: 515.45 (M+1).

N-methyl-4-((2-morpholino-6-(1-(2-(pyrrolidin-1-yl)ethyl)-1H-benzo[d]imidazol-6-yl)pyrimidin-4-yl)amino)benzamide: ¹H NMR (400 MHz, CD₃OD) δ: 8.31 (d, J=6.8 Hz, 2H), 7.97 (dd, J=8.7, 1.6 Hz, 1H), 7.77 (d, J=17.0 Hz, 5H), 6.67 (s, 1H), 4.58-4.48 (m, 2H), 3.95-3.82 (m, 8H), 3.04 (t, J=6.8 Hz, 2H), 2.92 (s, 3H), 2.65 (q, J=5.6, 4.1 Hz, 4H), 1.88-1.76 (m, 4H); HPLC purity: 93.48%; LCMS Calculated for C₂₉H₃₄N₈O₂: 526.28; observed: 527.50 (M+1).

N-methyl-4-((2-morpholino-6-(1-(2-morpholinoethyl)-1H-benzo[d]imidazol-6-yl)pyrimidin-4-yl)amino)benzamide: ¹H NMR (400 MHz, DMSO-d₆) δ: 11.49 (s, 1H), 10.07 (s, 1H), 9.59 (s, 1H), 8.64 (s, 1H), 8.32 (d, J=4.7 Hz, 1H), 8.14 (d, J=8.1 Hz, 1H), 7.99 (d, J=8.6 Hz, 1H), 7.88-7.75 (m, 4H), 6.86 (d, J=15.1 Hz, 1H), 5.04 (d, J=6.6 Hz, 2H), 3.99 (s, 3H), 3.89-3.71 (m, 11H), 3.64 (d, J=15.9 Hz, 4H), 2.77 (d, J=4.2 Hz, 3H); HPLC purity: 98.09%; LCMS Calculated for C₂₉H₃₄N₈O₃ (free base): 542.28; observed: 543.50 (M+1).

N-methyl-4-((2-morpholino-6-(1-(3-morpholinopropyl)-1H-benzo[d]imidazol-6-yl)pyrimidin-4-yl)amino)benzamide: ¹H NMR (400 MHz, CD₃OD) δ: 9.62 (s, 1H), 8.76 (s, 1H), 8.12-8.02 (m, 2H), 7.94-7.86 (m, 2H), 7.84-7.76 (m, 2H), 6.80 (s, 1H), 4.06 (d, J=13.1 Hz, 2H), 3.97 (dd, J=5.8, 3.6 Hz, 4H), 3.87 (dd, J=5.7, 3.7 Hz, 8 H), 3.56 (d, J=12.6 Hz, 2H), 3.46-3.32 (m, 2H), 3.30-3.15 (m, 2H), 2.94 (s, 3H), 2.73-2.56 (m, 2H); HPLC purity: 96.42%; LCMS Calculated for C₃₀H₃₆N₈O₃ (free base): 556.29; observed: 557.35 (M+1).

4-06-(1-(2-methoxyethyl)-1H-benzo[d]imidazol-6-yl)-2-morpholinopyrimidin-4-yl)amino)-N-methylbenzamide: ¹H NMR (400 MHz, DMSO-d₆) δ: 10.19 (s, 1H), 9.67 (s, 1H), 8.59 (s, 1H), 8.34 (d, J=5.4 Hz, 1H), 8.17 (d, J=8.7 Hz, 1H), 7.99 (d, J=8.6 Hz, 1H), 7.88-7.76 (m, 4H), 6.81 (s, 1H), 4.77 (t, J=5.1 Hz, 2H), 3.87-3.75(m, 8H), 3.28 (s, 3H), 2.77 (d, J=3.8 Hz, 3H); HPLC purity: 97.93%; LCMS Calculated for C₂₆H₂₉N₇O₃ (free base):487.23; observed: 488.35 (M+1).

4-((6-(1-cyclopropyl-1H-benzo[d]imidazol-5-yl)-2-morphohnopyrimidin-4-yl)amino)-N-methylbenzamide: ¹H NMR (400 MHz, DMSO-d₆) δ: 9.61 (s, 1H), 8.29 (dd, J=13.7, 5.5 Hz, 3H), 8.00-7.93 (m, 1H), 7.86-7.68 (m, 5H), 6.69 (s, 1H), 3.85-3.73 (m, 8H), 3.55 (tt, J=7.1, 3.6 Hz, 1H), 2.77 (d, J=4.4 Hz, 3H), 1.20-1.01 (m, 4H); HPLC purity: 95.52%; LCMS Calculated for C₂₆H₂₇N₇O₂:469.22; observed: 470.30 (M+1).

4-((6-(1-(cyclopropylmethyl)-1H-benzo[d]imidazol-5-yl)-2-morphohnopyrimidin-4-yl)amino)-N-methylbenzamide: ¹H NMR (400 MHz, DMSO-d₆) δ: 10.13 (t, J=11.4 Hz, 1H), 9.75 (d, J=8.7 Hz, 1H), 8.44 (s, 1H), 8.32 (d, J=5.2 Hz, 1H), 8.18 (t, J=6.2 Hz, 2H), 7.81 (q, J=8.5 Hz, 4H), 6.83 (d, J=6.1 Hz, 1H), 4.43 (dd, J=23.5, 7.1 Hz, 2H), 3.82-3.72 (m, 8H), 2.76 (d, J=3.8 Hz, 3H), 1.45 (d, J=8.8 Hz, 1H), 0.71-0.53 (m, 4H); HPLC purity: 98.78%; LCMS Calculated for C₂₇H₃₂N₈O₂ (free base):483.24; observed: 484.35 (M+1).

4-((6-(1-(2-(dimethylamino)ethyl)-1H-benzo[d]imidazol-5-yl)-2-morphohnopyrimidin-4-yl)amino)-N-methylbenzamide: ¹H NMR (400 MHz, DMSO-d₆) δ: 10.66 (s, 1H), 10.02 (s, 1H), 9.51 (s, 1H), 8.43 (s, 1H), 8.32 (q, J=4.5 Hz, 1H), 8.16 (s, 2H), 7.88-7.75 (m, 4H), 6.79 (s, 1H), 4.94 (t, J=6.3 Hz, 2H), 3.82-3.65 (m, 10H), 2.87 (d, J=4.1 Hz, 6H), 2.77 (d, J=4.2 Hz, 3H); HPLC purity: 98.94%; LCMS Calculated for C₂₇H₃₂N₈O₂ (free base): 500.26; observed: 501.45 (M+1).

N-methyl-4-((2-morphohno-6-(1-(3-(pyrradin-1-yl)propyl)-1H-benzo[d]imidazol-5-yl)pyrimidin-4-yl)amino)benzamide: ¹H NMR (400 MHz, DMSO-d₆) δ: 9.61 (s, 1H), 8.33-8.23 (m, 3H), 7.93 (dd, J=8.6, 1.6 Hz, 1H), 7.85-7.66 (m, 5H), 6.69 (s, 1H), 4.32 (t, J=6.9 Hz, 2H), 3.83-3.75 (m, 8H), 2.77 (d, J=4.4 Hz, 3H), 2.36 (dt, J=25.2, 6.1 Hz, 6H), 1.97 (p, J=7.1 Hz, 2H), 1.72-1.64 (m, 4H); HPLC purity: 97.36%; LCMS Calculated for C₃₀H₃₄N₈O₂:540.30; observed: 541.45 (M+1).

N-methyl-4-42-morphohno-6-(1-(2-morphohnoethyl)-1H-benzo[d]imidazol-5-yl)pyrimidin-4-yl)amino)benzamide: ¹H NMR (400 MHz, CD₃OD) δ: 8.36 (d, J=1.5 Hz, 1H), 8.29 (s, 1H), 8.07 (dd, J=8.5, 1.6 Hz, 1H), 7.79 (s, 4H), 7.68 (d, J=8.6 Hz, 1H), 6.65 (s, 1H), 4.45 (t, J=6.2 Hz, 2H), 3.91-3.82 (m, 8H), 3.69-3.62 (m, 4H), 2.92 (s, 3H), 2.82 (t, J=6.1 Hz, 2H), 2.52 (t, J=4.6 Hz, 4H); HPLC purity: 98.9%; LCMS Calculated for C₂₉H₃₄N₈O₃:542.28; observed: 543.45 (M+1).

N-methyl-4-((2-morpholino-6-(1-(3-morpholinopropyl)-1H-benzo[d]imidazol-5-yl)pyrimidin-4-yl)amino)benzamide: ¹H NMR (400 MHz, DMSO-d₆) δ: 9.60 (s, 1H), 8.33-8.22 (m, 3H), 7.93 (dd, J=8.6, 1.6 Hz, 1H), 7.85-7.67 (m, 5H), 6.69 (s, 1H), 4.32 (t, J=6.8 Hz, 2H), 3.85-3.69 (m, 8H), 3.55 (dd, J=6.3, 3.1 Hz, 4H), 2.77 (d, J=4.4 Hz, 3H), 2.29 (s, 4H), 2.21 (t, J=6.7 Hz, 2H), 1.98 (p, J=6.9 Hz, 2H); HPLC purity: 98.17%; LCMS Calculated for C₃₀H₃₆N₈O₃:556.29; observed: 557.55 (M+1).

4-((6-(1-cyclopropyl-2-methyl-1H-benzo[d]imidazol-6-yl)-2-morpholinopyrimidin-4-yl)amino)-N-methylbenzamide: ¹H NMR (400 MHz, DMSO-d₆) δ: 9.95 (s, 1H), 8.40 (d, J=1.6 Hz, 1H), 8.31 (q, J=4.5 Hz, 1H), 8.17 (dd, J=8.5, 1.6 Hz, 1H), 7.94-7.73 (m, 5H), 6.81 (s, 1H), 3.83-3.74 (m, 8H), 3.63 (tt, J=7.3, 4.0 Hz, 1H), 2.89 (s, 3H), 2.77 (d, J=4.2 Hz, 3H), 1.41-1.21 (m, 4H); HPLC purity: 93.51%; LCMS Calculated for C₂₇H₂₉N₇O₂ (free base):483.24; observed: 484.40 (M+1).

4-((6-(1-(cyclopropylmethyl)-2-methyl-1H-benzo[d]imidazol-6-yl)-2-morpholinopyrimidin-4-yl)amino)-N-methylbenzamide: ¹H NMR (400 MHz, DMSO-d₆) δ: 9.83 (s, 1H), 8.53 (d, J=1.5 Hz, 1H), 8.29 (q, J=4.5 Hz, 1H), 8.19 (d, J=8.6 Hz, 1H), 7.94-7.74 (m, 5H), 6.76 (s, 1H), 4.47 (d, J=7.1 Hz, 2H), 3.86-3.74 (m, 8H), 2.91 (s, 3H), 2.77 (d, J=4.4 Hz, 3H), 1.43-1.31 (m, 1H), 0.67-0.56 (m, 4H); HPLC purity: 97.2%; LCMS Calculated for C₂₈H₃₁N₇O₂ (free base):497.25; observed: 498.45 (M+1).

4-((6-(1-cyclobutyl-2-methyl-1H-benzo[d]imidazol-6-yl)-2-morpholinopyrimidin-4-yl)amino)-N-methylbenzamide ¹H NMR (400 MHz, CD₃OD) δ: 8.42 (s, 1H), 8.05-7.86 (m, 4H), 7.78 (d, J=8.3 Hz, 2H), 6.69 (s, 1H), 5.33 (t, J=8.6 Hz, 1H), 3.96-3.83 (m, 8H), 3.09-2.76 (m, 10H), 2.21-2.07 (m, 2H); HPLC purity: 97.73%; LCMS Calculated for C₂₈H₃₁N₇O₂ (free base):497.25; observed: 498.45 (M+1).

4-((6-(1,2-dimethyl-1H-benzo[d]imidazol-6-yl)-2-morpholinopyrimidin-4-yl)amino)-N-methylbenzamide: ¹H NMR (400 MHz, DMSO-d₆) δ: 9.62 (s,1H), 8.27 (d, J=4.0 Hz, 1H),8.11 (s,1H),7.83-7.75 (m, 5H), 7.58 (d, J=9.2 Hz, 1H), 6.70 (s,1H),3.80-3.73 (m, 11H), 2.77 (d, J=4.0 Hz, 3H), 2.56 (s, 3H); HPLC purity: 98.73%; LCMS Calculated for C₂₅H₂₇N₇O₂: 457.22; observed: 458.30 (M+1).

4-((6-(1-cyclopropyl4H-indazol-6-yl)-2-morpholinopyrimidin-4-yl)amino)-N-methylbenzamide: ¹H NMR (400 MHz, DMSO-d₆) δ: 10.03 (s, 1H), 8.30 (d, J=21.2 Hz, 2H), 8.09 (s, 1H), 7.91-7.73 (m, 6H), 6.79 (s, 1H), 3.84-3.75 (m, 9H), 2.77 (d, J=4.2 Hz, 3H), 1.17 (d, J=7.0 Hz, 4H); HPLC purity: 95.86%; LCMS Calculated for C₂₆H₂₇N₇O₂ (free base): 469.22; Observed: 470.40 (M+1).

4-((6-(1-(cyclopropylmethyl)-1H-indazol-6-yl)-2-morpholinopyrimidin-4-yl)amino)-N-methylbenzamide: 1H NMR (400 MHz, DMSO-d₆) δ: 10.35 (s, 1H), 8.35 (d, J=5.4 Hz, 1H), 8.27 (s, 1H), 8.15 (s, 1H), 7.88 (dd, J=17.3, 8.4 Hz, 3H), 7.79 (d, J=8.4 Hz, 2H), 7.67 (d, J=8.4 Hz, 1H), 6.78 (s, 1H), 4.40 (d, J=7.0 Hz, 2H), 3.83-3.75 (m, 8H), 2.77 (d, J=4.0 Hz, 3H),1.32 (p, J=6.4 Hz, 1H), 0.57-0.40 (m, 4H); HPLC purity: 96.14%; LCMS Calculated for C₂₇H₂₉N₇O₂ (free base): 483.24; Observed: 484.30 (M+1).

4-((6-(1-cyclobutyl-1H-indazol-6-yl)-2-morpholinopyrimidin-4-yl)amino)-N-methylbenzamide: ¹H NMR (400 MHz, DMSO-d₆) δ: 10.01 (s, 1H),8.36 (s, 1H), 8.31-8.18 (m, 2H), 7.87 (dd, J=14.5, 8.4 Hz, 3H), 7.77 (d, J=8.5 Hz, 3H), 6.78 (s, 1H), 5.18-5.24 (m,1H), 3.84-3.75 (m, 8H), 2.77 (d, J=4.1 Hz, 3H), 2.66 (qd, J=9.8, 2.5 Hz, 2H),2.28-2.46 (m, 2H), 1.98-1.85 (m, 2H); HPLC purity: 99.74%; LCMS Calculated for C₂₇H₂₉N₇O₂(free base): 483.24; Observed: 484.30 (M+1).

4-((6-(1-(2-(dimethylamino)ethyl)-1H-indazol-6-yl)-2-morpholinopyrimidin-4-yl)amino)-N-methyl benzamide: ¹H NMR (400 MHz, CD₃OD) δ: 8.29 (d, J=1.3 Hz, 1H), 8.07 (d, J=0.9 Hz, 1H), 7.87-7.77 (m, 6H), 6.68 (s, 1H), 4.63 (t, J=6.9 Hz, 2H), 3.92-3.81 (m, 8H), 2.91 (d, J=8.0 Hz, 5H), 2.32 (s, 6H); HPLC purity: 99.9%; LCMS Calculated for C₂₇H₃₂N₈O₂: 500.26; Observed: 501.35 (M+1).

4-((6-(2-(2-(dimethylamino)ethyl)-2H-indazol-6-yl)-2-morpholinopyrimidin-4-yl)amino)-N-methylbenzamide: Regioisomer of the above compound obtained by chromatography after the final step. ¹H NMR (400 MHz, DMSO-d₆) δ: 9.64 (s, 1H), 8.43 (s, 1H), 8.28 (s, 2H), 7.79 (dt, J=11.2, 8.4 Hz, 5H), 7.66 (d, J=8.9 Hz, 1H), 6.69 (s, 1H), 4.54 (t, J=6.3 Hz, 2H), 3.85-3.73 (m, 8H), 2.87-2.74 (m, 5H), 2.18 (s, 6H); HPLC purity: 99.74%; LCMS Calculated for C₂₇H₃₂N₈O₂: 500.26; Observed: 501.35 (M+1).

N-methyl-4-42-morpholino-6-(1-(2-(pyrrolidin-l-yl)ethyl)-1H-indazol-6-yl)pyrimidin-4-yl)amino)benzamide: ¹H NMR (400 MHz, DMSO-d₆) δ: 9.68 (s, 1H), 8.27 (s, 2H), 8.11 (s, 1H), 7.88-7.72 (m, 6H), 6.74 (s, 1H), 4.60 (t, J=6.5 Hz, 2H), 3.82-3.73 (m, 8H), 2.91 (t, J=6.1 Hz, 2H), 2.77 (d, J=4.4 Hz, 3H),2.56-2.40 (m, 4H), 1.63 (s, 4H); HPLC purity: 99.05%; LCMS Calculated for C₂₉H₃₄N₈O₂: 526.28; Observed: 527.50 (M+1).

N-methyl-4-((2-morpholino-6-(1-(3-(pyrrolidin-1-yl)propyl)-1H-indazol-6-yl)pyrimidin-4-yl)amino)benzamide: ¹H NMR (400 MHz, CD₃OD) δ: 8.28 (q, J=1.0 Hz, 1H), 8.06 (d, J=0.9 Hz, 1H), 7.81 (d, J=9.9 Hz, 6H), 6.68 (s, 1H), 4.57 (t, J=6.7 Hz, 2H), 3.96-3.78 (m, 8H), 2.92 (s, 3H), 2.56-2.45 (m, 6H), 2.18 (p, J=6.8 Hz, 2H), 1.79 (p, J=3.2 Hz, 4H); HPLC purity: 98.60%; LCMS Calculated for C₃₀H₃₆N₈O₂: 540.30; Observed: 541.45 (M+1).

N-methyl-4-((2-morpholino-6-(1-(2-morpholinoethyl)-1H-indazol-6-yl)pyrimidin-4-yl)amino)benzamide: ¹H NMR (400 MHz, DMSO-d₆) δ: 10.98 (s, 1H), 10.06 (s, 1H), 8.42 (s, 1H), 8.33 (d, J=4.9 Hz, 1H), 8.26 (s, 1H), 7.94 (d, J=8.4 Hz, 1H), 7.88-7.70 (m, 5H), 6.78 (s, 1H), 5.01 (t, J=6.7 Hz, 2H), 3.99 (d, J=12.7 Hz, 2H), 3.89-3.82 (m, 4H), 3.81-3.62 (m, 8H), 3.58-3.46 (m, 2H), 3.18 (d, J=9.9 Hz, 2H), 2.77 (d, J=4.3 Hz, 3H); HPLC purity: 97.56%; LCMS Calculated for C₂₉H₃₄N₈O₃ (free base): 542.28; Observed: 543.40 (M+1).

4-((6-(1-(2-methoxyethyl)-1H-indazol-6-yl)-2-morpholinopyrimidin-4-yl)amino)-N-methylbenzamide: 1H NMR (400 MHz, DMSO-d₆) δ: 10.63 (s, 1H), 8.38 (d, J=5.8 Hz, 1H), 8.24 (s, 1H), 8.18 (s, 1H), 7.89 (dd, J=13.6, 8.4 Hz, 3H), 7.80 (d, J=8.5 Hz, 2H), 7.62 (d, J=8.4 Hz, 1H), 6.82 (s, 1H), 6.00 (s, 1H), 4.66 (t, J=5.4 Hz, 2H), 3.89-3.72 (m, 10H), 3.21 (s, 3H), 2.78 (d, J=3.8 Hz, 3H); HPLC purity: 98.99%; LCMS Calculated for C₂₆H₂₉N₇O₃ (free base): 487.23; Observed: 488.30 (M+1).

4-((6-(1-cyclopropyl-1H-indazol-5-yl)-2-morpholinopyrimidin-4-yl)amino)-N-methylbenzamide: 1H NMR (400 MHz, CD3OD) δ: 8.34-8.27 (m, 1H), 8.17 (d, J=3.7 Hz, 1H), 7.96-7.74 (m, 6H), 6.60 (d, J=4.0 Hz, 1H), 3.95-3.82 (m, 8H), 3.77 (tq, J=7.7, 3.9 Hz, 1H), 2.93 (d, J=3.9 Hz, 3H), 1.24 (dddt, J=10.2, 6.5, 4.2, 2.0 Hz, 4H); HPLC purity: 99.02%; LCMS Calculated for C₂₆H₂₇N₇O₂ (free base): 469.22; Observed: 470.30 (M+1).

N-methyl-4-((2-morpholino-6-(1-(2-morpholinoethyl)-1H-indazol-5-yl)pyrimidin-4-yl)amino)benzamide: ¹H NMR (400 MHz, DMSO-d₆) δ: 11.60 (s, 1H), 10.42 (s, 1H), 8.44 (s, 1H), 8.36 (d, J=4.5 Hz, 2H), 7.98 (s, 2H), 7.90-7.76 (m, 4H), 6.80 (s, 1H), 5.00 (t, J=7.0 Hz, 2H), 3.98 (dd, J=12.1, 3.4 Hz, 2H), 3.88-3.71 (m, 10H), 3.65 (t, J=7.0 Hz, 2H), 3.52-3.46 (m, 2H), 3.17 (q, J=8.3, 6.7 Hz, 2H), 2.83 (t, 3H); HPLC purity: 97.22%; LCMS Calculated for C₂₉H₃₄N₈O₃ (free base): 542.28; Observed: 543.45 (M+1).

N-methyl-4-((2-morpholino-6-(1-(3-morpholinopropyl)-1H-indazol-5-yl)pyrimidin-4-yl)amino)benzamide: ¹H NMR (400 MHz, DMSO-d₆) δ: δ 9.63 (s, 1H), 8.46 (s, 1H), 8.28 (q, J=4.4 Hz, 1H), 8.21 (s, 1H), 8.01 (d, J=9.0 Hz, 1H), 7.86-7.72 (m, 5H), 6.68 (s, 1H), 4.47 (t, J=6.6 Hz, 2H), 3.85-3.77 (m, 4H), 3.77-3.68 (m, 4H), 3.54 (t, J=4.4 Hz, 4H), 2.77 (d, J=4.4 Hz, 3H), 2.29-2.15 (m, 6H), 2.00 (p, J=6.8 Hz, 2H); HPLC purity: 97.86%; LCMS Calculated for C₃₀H₃₆N₈O₃: 556.29; Observed: 557.45 (M+1).

N-methyl-4-((6-(1-methyl-1H-indazol-5-yl)-2-morpholinopyrimidin-4-yl)amino)benzamide: ¹H NMR (400 MHz, DMSO-d₆) δ: 10.24 (s, 1H), 8.42 (s, 1H), 8.34 (d, J=4.9 Hz, 1H), 8.22 (s, 1H), 7.95 (d, J=9.0 Hz, 1H), 7.89-7.71 (m, 5H), 6.72 (s, 1H), 4.10 (s, 3H), 3.83-3.74 (m, 8H), 2.77 (d, J=4.2 Hz, 3H); HPLC purity: 95.46%; LCMS Calculated for C₂₄H₂₅N₇O₂ (freebase): 443.21; Observed: 444.25 (M+1).

N-(4-chlorophenyl)-6-(2-methyl-1-(2-morpholinoethyl)-1H-benzo[d]imidazol-5-yl)-2-morpholinopyrimidin-4-amine: ¹H NMR (400 MHz, Methanol-d₄) δ 8.38 (d, J=8.7 Hz, 1H), 8.28 (s, 1H), 8.05 (dd, J=8.8, 1.6 Hz, 1H), 7.65 (d, J=8.6 Hz, 2H), 7.53-7.40 (m, 2H), 6.60 (s, 1H), 5.10 (t, J=7.8 Hz, 2H), 4.05 (s, 4H), 3.93-3.66 (m, 8H), 3.64 (s, 4H), 3.47-3.32 (m, 2H), 3.06 (s, 3H)HPLC purity: 96.61%; LCMS Calculated for C₂₈H₃₂ClN₇O:518.05:observed: 534.40(M+1).

N-methyl-4-((2-morpholino-6-(1-(3-morpholinopropyl)-1H-indazol-6-yl)pyrimidin-4-yl)amino)benzamide: ¹H NMR (400 MHz, DMSO-d₆) δ: 9.68 (s, 1H), 8.32-8.23 (m, 2H), 8.13 (s, 1H), 7.88-7.70 (m, 6H), 6.74 (s, 1H), 4.53 (t, J=6.3 Hz, 2H), 3.87-3.77 (m, 4H), 3.77-3.70 (m, 4H), 3.51 (t, J=4.4 Hz, 4H), 2.77 (d, J=4.4 Hz, 3H), 2.25-2.12 (m, 6H), 2.07-1.97 (m, 2H); HPLC purity: 97.44%; LCMS Calculated for C₃₀H₃₆N₈O₃: 556.29; Observed: 557.40 (M+1).

### Examples 347-351

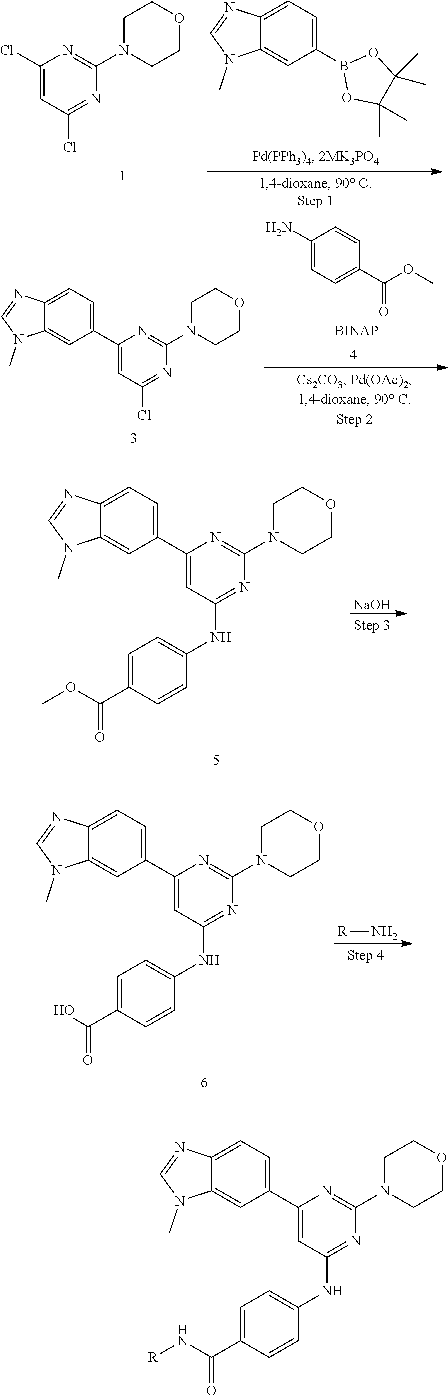

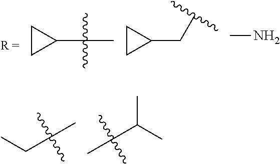

### Step 1: Synthesis of 4-(4-chloro-6-(1-methyl-1H-benzo[d]imidazol-6-yl)pyrimidin-2-yl)morpholine (3)

The title compound has been synthesized by following the general procedure described above for Suzuki coupling by using compound 1 and Boronate ester 2. LCMS (m/z): 330.05 (M+1).

### Step 2: Synthesis of methyl 4-((6-(1-methyl-1H-benzo[d]imidazol-6-yl)-2-morpholinopyrimidin-4-yl) amino)benzoate (5)

The title compound (crude) has been synthesized by following the general procedure described above for Buchwald Coupling by using compound3and methyl 4-aminobenzoate 4. LCMS (m/z): 445.20 (M+1).

### Step 3: Synthesis of 4-((6-(1-methyl-1H-benzo[d]imidazol-6-yl)-2-morpholinopyrimidin-4-yl)amino) benzoic acid (6)

The title compound has been synthesized by following the procedure described above for ester hydrolysis by using compound 5 and the crude product has been used as such for the next step. LCMS (m/z): 431.25 (M+1).

The following compounds were prepared using the general procedure described above for Amide coupling by using compound 6 and the appropriate amine.

N-cyclopropyl-4-((6-(1-methyl-1H-benzo[d]imidazol-6-yl)-2-morpholinopyrimidin-4-yl) amino)benzamide: The title compound has been synthesized by following. ¹H NMR (400 MHz, DMSO-d₆) δ: 9.64 (s, 1H), 8.30-8.21 (m, 2H), 8.19 (s, 1H), 7.94-7.86 (m, 1H), 7.84-7.69 (m, 5H), 6.73 (s, 1H), 3.92 (s, 3H), 3.81-3.73 (m, 8H), 2.86-2.80 (m, 1H), 0.67 (dt, J=7.0, 3.3 Hz, 2H), 0.56 (dt, J=7.3, 4.6 Hz, 2H); HPLC purity: 95.27%; LCMS Calculated for C₂₆H₂₇N₇O₂(free base): 469.22; observed: 470.30 (M+1).

N-(cyclopropylmethyl)-4-((6-(1-methyl-1H-benzo[d]imidazol-6-yl)-2-morpholinopyrimidin-4-yl)amino)benzamide: The title compound has been synthesized by following the general procedure described above for Amide coupling by using compound 6 and cyclopropyl methylamine. ¹H NMR (400 MHz, DMSO-d₆) δ: 9.66 (s, 1H), 8.40 (t, J=5.8 Hz, 1H), 8.28 (s, 1H), 8.20 (s, 1H), 7.88 (dd, J=21.1, 8.5 Hz, 3H), 7.76 (dd, J=17.0, 8.5 Hz, 3H), 6.73 (s, 1H), 3.92 (s, 3H), 3.82-3.74 (m, 8H),3.13 (t, J=6.3 Hz, 2H), 1.07-0.97 (m, 1H), 0.47-0.38 (m, 2H), 0.22-0.20 (m, 2H); HPLC purity: 99.17%; LCMS Calculated for C₂₇H₂₉N₇O₂: 483.24; observed: 484.30(M+1).

4-((6-(1-methyl-1H-1-benzo[d]imidazol-6-yl)-2-morpholinopyrimidin-4-yl)amino)benzamide: To a stirred solution of acid 6 (150mg, 1 eq) in DMF (2 mL) CDI (67 mg, 1.2 eq) was added and the resulting mixture stirred at 60° C. for 1 h. Reaction mixture was cooled to room temperature, ammonium hydroxide (0.49 mL, 10 eq) was added and the reaction mixture was stirred for 1 h at room temperature. After completion of the reaction, water was added and extracted with ethyl acetate (2×25 mL). Combined organic extracts were washed with brine, dried over anhydrous sodium sulfate and evaporated under reduced pressure. The crude product was purified by preparative HPLC to afford the desired product. ¹H NMR (400 MHz, DMSO-d₆) δ: 9.65 (s, 1H), 8.28 (s, 1H), 8.20 (d, J=1.6 Hz, 1H), 7.94-7.70 (m, 7H), 7.15 (s, 1H), 6.73 (s, 1H), 3.92 (s, 3H), 3.86-3.70 (m, 8H); HPLC purity: 98.97%; LCMS Calculated for C₂₃H₂₃N₇O₂: 429.19; observed: 430.25 (M+1).

N-ethyl-4-((6-(1-methyl-1H-1-benzo[d]imidazol-6-yl)-2-morpholinopyrimidin-4-yl)amino) benzamide: The title compound has been synthesized by following the general procedure described above for Amide coupling by using compound 6 and ethyl amine. ¹H NMR (400 MHz, DMSO-d₆) δ: 9.83 (s, 1H), 9.45 (s, 1H), 8.49 (d, J=1.5 Hz, 1H), 8.33 (t, J=5.6 Hz, 1H), 8.18 (d, J=8.8 Hz, 1H), 7.95 (d, J=8.6 Hz, 1H), 7.88-7.74 (m, 4H), 6.78 (s, 1H), 4.12 (s, 3H), 3.83-3.74 (m, 8H), 3.33-3.22 (m, 2H), 1.12 (t, 3H); HPLC purity: 95.41%; LCMS Calculated for C₂₅H₂₇N₇O₂ (free base): 457.22; observed: 458.40 (M+1).

N-isopropyl-4-((6-(1-methyl-1H-benzo[d]imidazol-6-yl)-2-morpholinopyrimidin-4-yl) amino)benzamide: The title compound has been synthesized by following the general procedure described above for Amide coupling by using compound 6 and Isopropyl amine. ¹H NMR (400 MHz, CD3OD) δ: 9.59 (s, 1H), 8.55 (s, 1H), 8.08 (s, 2H), 7.95-7.87 (m, 2H), 7.77 (d, J=8.2 Hz, 2H), 6.69 (s, 1H), 4.29-4.16 (m, 4H), 3.98-3.83 (m, 8H), 1.36-1.23 (m, 6H); HPLC purity: 95.24%; LCMS Calculated for C₂₆H₂₉N₇O₂ (free base): 471.24; observed: 472.40(M+1).

### Example 352

### Synthesis of 4-((6-(1-methyl-1H-benzo[d]imidazol-6-yl)-2-morpholinopyrimidin-4-yl)amino) benzonitrile

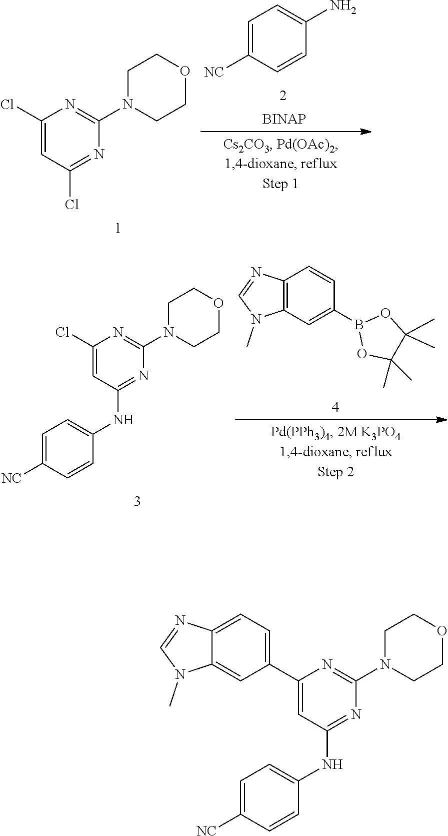

### Step 1: Synthesis of 4-((6-chloro-2-morpholinopyrimidin-4-yl)amino)benzonitrile (3)

The title compound (crude product) has been synthesized by following the general procedure described above for Buchwald coupling by using chloro compound 1 and 4-cyano aniline2. LCMS (m/z): 315.90 (M+1).

### Step 2: synthesis of 4-((6-(1-methyl-1H-benzo[d]imidazol-6-yl)-2-morpholinopyrimidin-4-yl)amino)benzonitrile

The title compound has been synthesized by following the general procedure described above for Suzuki coupling by using compound 3 and Boronate ester 4. ¹H NMR (400 MHz, DMSO-d₆) δ: 9.91 (s, 1H), 8.29 (s, 1H), 8.20 (d, J=1.6 Hz, 1H), 7.90 (dd, J=11.9, 8.5 Hz, 3H), 7.75 (dd, J=8.6, 6.4 Hz, 3H), 6.76 (s, 1H), 3.92 (s, 3H), 3.85-3.70 (m, 8H); HPLC purity: 97.54%; LCMS Calculated for C₂₃H₂₁N₇O: 411.18; Observed: 412.30 (M+1).

### Examples 353-357

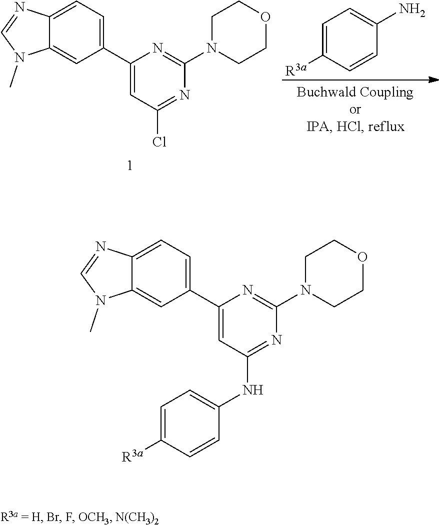

6-(1-methyl-1H-benzo [d]imidazol-6-yl)-2-morpholino-N-phenylpyrimidin-4-amine: The title compound has been synthesized by following the general procedure described above for displacement reaction (IPA, Conc. HCl) by using chloro compound 1 and aniline. ¹H NMR (400 MHz, DMSO-d₆) δ: 9.82 (s, 1H), 9.61 (s, 1H), 8.50 (d, J=1.5 Hz, 1H), 8.17 (d, J=8.8 Hz, 1H), 7.98 (d, J=8.6 Hz, 1H), 7.70 (d, J=8.0 Hz, 2H), 7.35 (t, J=7.9 Hz, 2H), 7.03 (t, J=7.4 Hz, 1H), 6.77 (s, 1H), 4.14 (s, 3H), 3.83-3.73 (m, 8H); HPLC purity: 99.08%; LCMS Calculated for C₂₂H₂₂N₆O (free base): 386.19; Observed: 387.25 (M+1).

N-(4-bromophenyl)-6-(1-methyl-1H-benzo[d]imidazol-6-yl)-2-morpholinopyrimidin-4-amine: The title compound has been synthesized by following the general procedure described above for displacement reaction (IPA, Conc. HCl) by using chloro compound 1 and 4-bromo aniline. ¹H NMR (400 MHz, DMSO-d₆) δ: 9.95 (s, 1H), 9.61 (s, 1H), 8.48 (s, 1H), 8.15 (d, J=8.9 Hz, 1H), 7.95 (d, J=8.6 Hz, 1H), 7.70-7.59 (m, 2H), 7.52-7.43 (m, 2H), 6.75 (s, 1H), 4.11 (s, 3H), 3.82-3.66 (m, 8H); HPLC purity: 95.60%; LCMS Calculated for C₂₂H₂₁BrN₆O (free base): 464.10; Observed: 465.25 (M+1).

N-(4-fluorophenyl)-6-(1-methyl-1H-benzo[d]imidazol-6-yl)-2-morpholinopyrimidin-4-amine: ¹H NMR (400 MHz, DMSO-d₆) δ: 10.06 (s, 1H), 9.66 (s, 1H), 8.51 (s, 1H), 8.15 (d, J=8.7 Hz, 1H), 7.99 (d, J=8.6 Hz, 1H), 7.76-7.67 (m, 2H), 7.20 (t, J=8.8 Hz, 2H), 6.76 (s, 1H), 4.14 (s, 3H), 3.82-3.70 (m, 8H); HPLC purity: 98.81%; LCMS Calculated for C₂₂H₂₁FN₆O (free base): 404.18; Observed: 405.25 (M+1).

N-(4-methoxyphenyl)-6-(1-methyl-1H-benzo[d]imidazol-6-yl)-2-morpholinopyrimidin-4-amine The title compound has been synthesized by following the general procedure described above for displacement reaction (IPA, Conc. HCl) by using chloro compound 1 and 4-methoxy aniline. ¹H NMR (400 MHz, DMSO-d₆) δ: 9.19 (s, 1H), 8.26 (s, 1H), 8.19-8.14 (m, 1H), 7.86 (dd, J=8.5, 1.6 Hz, 1H), 7.71 (d, J=8.5 Hz, 1H), 7.61-7.53 (m, 2H), 6.96-6.87 (m, 2H), 6.60 (s, 1H), 3.91 (s, 3H), 3.82-3.67 (m, 11H); HPLC purity: 99.85%; LCMS Calculated for C₂₃H₂₄N₆O₂: 416.20; Observed: 417.30 (M+1).

N1,N1-dimethyl-N4-(6-(1-methyl-1H-benzo[d]imidazol-6-yl)-2-morpholinopyrimidin-4-yl)benzene-1,4-diamine: The title compound has been synthesized by following the general procedure described above for displacement reaction (IPA, Conc. HCl) by using chloro compound 1 and N1,N1-dimethylbenzene-1,4-diamine ¹H NMR (400 MHz, DMSO-d₆) δ: 10.21 (s, 1H), 9.65 (s, 1H), 8.49 (s, 1H), 8.14 (d, J=8.8 Hz, 1H), 7.96 (d, J=8.7 Hz, 1H), 7.85 (d, J=8.6 Hz, 2H), 7.69 (d, J=8.6 Hz, 2H), 6.81 (s, 1H), 4.11 (s, 3H), 3.84-3.71 (m, 8H), 3.06 (s, 6H); HPLC purity: 99.19%; LCMS Calculated for C₂₄H₂₇N₇O (free base): 429.23; Observed: 430.35 (M+1).

### Example 358

### Synthesis of N-(4-((6-(1-methyl-1H-benzo[d]imidazol-6-yl)-2-morpholinopyrimidin-4-yl) amino) phenyl)acetamide

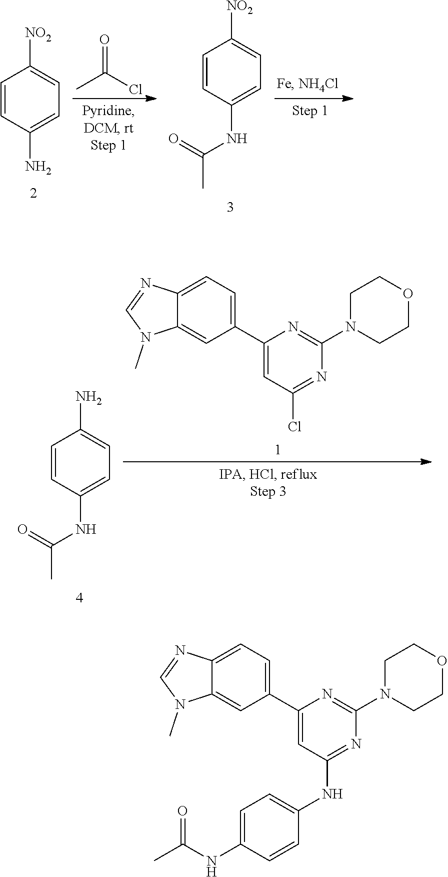

### Step 1: Synthesis of N-(4-nitrophenyl)acetamide (3)

To a stirred solution of compound 2 (3 g, 1 eq) in pyridine (12 mL) and dichloromethane (30 mL), acetyl chloride(2 g, 1.2 eq)was added at 0° C. and stirred at room temperature for 1 h. The progress of the reaction was monitored by TLC. After completion of the reaction, the reaction mixture was evaporated to dryness. The residue was washed with water, brine, dried over anhydrous sodium sulfate and evaporated under reduced pressure to afford the title compound 3.

### Step 2: Synthesis of N-(4-aminophenyl)acetamide (4)

To a stirred solution of compound 3 (3 g, 1 eq) in ethanol (30 mL), iron powder (4.6 g, 5 eq), water (10 mL) and ammonium chloride (4.4 g, 5 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (100 mL), washed with brine, dried over anhydrous sodium sulfate and evaporated under reduced pressure to afford the title compound 4. LCMS (m/z): 151.00 (M+1).

### Step 3: Synthesis of N-(4-((6-(1-methyl-1H-benzo[d]imidazol-6-yl)-2-morpholinopyrimidin-4-yl) amino) phenyl)acetamide

The title compound has been synthesized by following the general procedure described above for displacement reaction (IPA, Conc. HCl) by using chloro compound 1 and N-(4-aminophenyl)acetamide. ¹H NMR (400 MHz, D20) δ: 9.18 (s, 1H), 8.08 (d, J=1.5 Hz, 1H), 7.89 (d, J=8.6 Hz, 1H), 7.78 (dd, J=8.7, 1.6 Hz, 1H), 7.41-7.33 (m, 2H), 7.23 (d, J=8.5 Hz, 2H), 6.25 (s, 1H), 4.11 (s, 3H), 3.88-3.75 (m, 8H), 1.89 (s, 3H); HPLC purity: 97.15%; LCMS Calculated for C₂₄H₂₅N₇O₂ (free base): 443.21; Observed: 444.35 (M+1).

### Example 359

### Synthesis of N-(4-((6-(1-methyl-1H-benzo[d]imidazole-6-yl)-2-morpholinopyrimidin-4-yl)amino) phenyl) methanesulfonamide

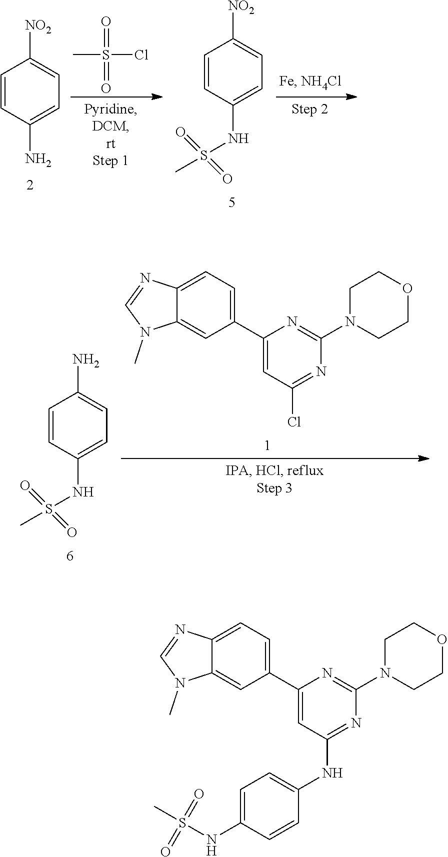

### Step 1: Synthesis of N-(4-nitrophenyl) methane sulfonamide (5)

To a stirred solution of compound 2 (2 g, 1.2 eq) in dichloromethane (20 mL), methane sulfonyl chloride (3 g, 1 eq) and pyridine were added at 0° C. and stirred at same temperature for 2 h. The progress of the reaction was monitored by TLC. After completion of the reaction, the reaction mixture was diluted with water (20 mL) and extracted with dichloromethane (2×25 mL). Combined organic extracts were washed with brine, dried over anhydrous sodium sulfate and evaporated under reduced pressure to afford the crude product 5.

### Step 2: Synthesis of N-(4-aminophenyl) methane sulfonamide (6)

To a stirred solution of compound 5 (0.8 g, 1 eq) in ethanol (20 mL), iron powder (1.03 g, 5 eq), water (20 mL) and ammonium chloride (0.98 g,5 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (50 mL) and washed with brine. The organic extract was dried over anhydrous sodium sulfate and evaporated under reduced pressure to afford the title compound 6.
¹NMR (400 MHz, DMSO-d₆) δ: 8.89 (s, 1H), 6.88 (d, J=8.0 Hz, 2H), 6.50(d, J=8.8 Hz, 2H), 5.01 (s, 2H), 2.79(s, 3H).

### Step 3: Synthesis of N-(4-((6-(1-methyl-1H-benzo[d]imidazole-6-yl)-2-morpholinopyrimidin-4-yl) amino)phenyl) methanesulfonamide

The title compound has been synthesized by following the general procedure described above for displacement reaction (IPA, Conc. HCl) by using chloro compound 1 and N-(4-aminophenyl) methanesulfonamide. ¹H NMR (400 MHz, DMSO-d₆) δ: 9.83 (s, 1H), 9.59 (d, J=3.6 Hz, 2H), 8.50 (s, 1H), 8.16 (d, J=8.7 Hz, 1H), 7.98 (d, J=8.6 Hz, 1H), 7.67 (d, J=8.6 Hz, 2H), 7.21 (d, J=8.6 Hz, 2H), 6.73 (s, 1H), 4.14 (s, 3H), 3.82-3.74 (m, 8H), 2.96 (d, J=1.2 Hz, 3H); HPLC purity: 97.02%; LCMS Calculated for C₂₃H₂₅N₇O₃S (free base): 479.17; Observed: 480.40 (M+1).

### Example 360

### Synthesis of 4-((6-(1-methyl-1H-benzo[d]imidazol-6-yl)-2-morpholinopyrimidin-4-yl) amino) benzene sulfonamide

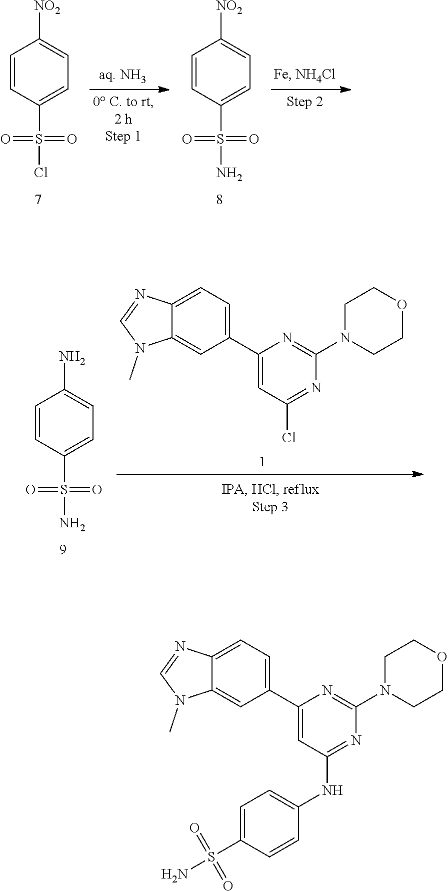

### Step 1: Synthesis of 4-nitrobenzenesulfonamide (8)

To a stirred solution of compound 7 (2 g, 1 eq) in THF (20 mL), aq. ammonia (9.3 mL) was added slowly at 0° C. and stirred at room temperature for 2 h. The progress of the reaction was monitored by TLC. After completion of the reaction, the reaction mixture was evaporated to dryness. The residue was dissolved in water (25 mL) and extracted with ethyl acetate (3×25 mL). Combined organic extracts were washed with brine, dried over anhydrous sodium sulfate and evaporated under reduced pressure to afford the crude product 8. ¹H NMR (400 MHz, DMSO-d₆) δ: 8.42-8.40 (m, 2H), 8.07-8.05 (m, 2H), 7.73 (s, 2H).

### Step 2: Synthesis of 4-aminobenzenesulfonamide (9)

To a stirred solution of compound 8 (0.8 g, 1 eq) in ethanol (20 mL), iron powder (1.03 g, 5 eq), water (10 mL) and ammonium chloride (0.98 g, 5 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (50 mL) and washed with brine, dried over anhydrous sodium sulfate and evaporated under reduced pressure to afford the title compound 9. ¹H NMR (400 MHz, DMSO-d₆) δ: 7.42-7.40 (m, 2H), 6.90 (s, 2H), 6.60-6.58 (m, 2H), 5.80 (s, 2H).

### Step 3: 4-((6-(1-methyl-1H-benzo[d]imidazol-6-yl)-2-morpholinopyrimidin-4-yl) amino) benzene sulfonamide

The title compound has been synthesized by following the general procedure described above for displacement reaction (IPA, Conc. HCl) by using chloro compound 1 and 4-aminobenzenesulfonamide. ¹H NMR (400 MHz, DMSO-d₆) δ: 9.80 (s, 1H), 8.28 (s, 1H), 8.20 (d, J=1.7 Hz, 1H), 7.94-7.80 (m, 3H), 7.75 (dd, J=12.4, 8.7 Hz, 3H), 7.19 (s, 2H), 6.75 (s, 1H), 3.92 (s, 3H), 3.85-3.74 (m, 8H); HPLC purity: 98.48%; LCMS Calculated for C₂₂H₂₃N₇O₃S: 465.16; Observed: 466.30 (M+1).

### Example 361

### Synthesis of N1-(6-(1-methyl-1H-benzo[d]imidazol-6-yl)-2-morpholinopyrimidin-4-yl)benzene-1,4-diamine

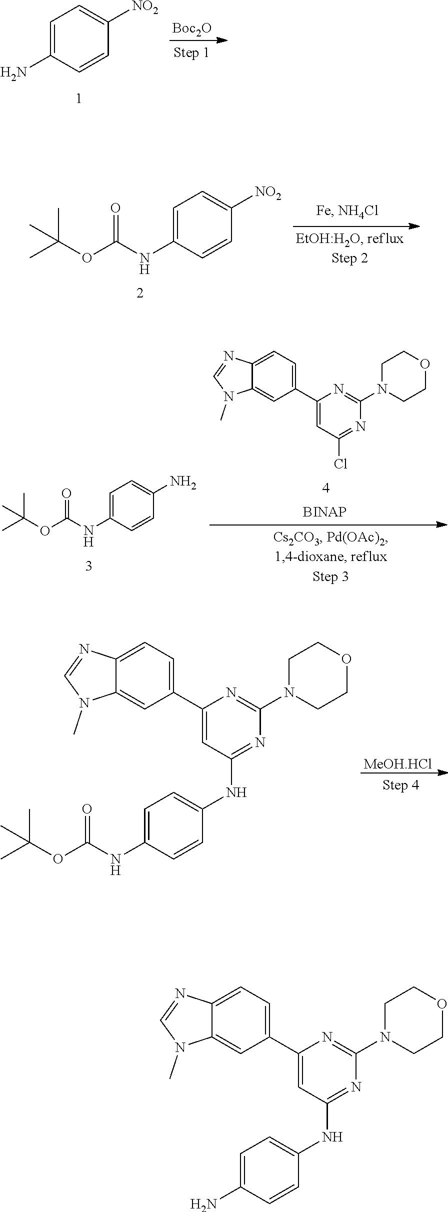

### Step 1: Synthesis of tert-butyl (4-nitrophenyl)carbamate (2)

To a stirred solution of compound 1 (4 g, 1 eq) in dichloromethane (60 mL), TEA (5.85 g, 2 eq) and DMAP (1.76 g, 0.5 eq) were added followed by the addition of Boc anhydride (9.48 g, 1.5 eq). The reaction mixture was stirred at room temperature for 18 h. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 30% EtOAc-hexane to afford the title compound 2. ¹H NMR (400 MHz, DMSO-d₆) δ: 10.12 (s, 1H), 8.17 (d, J=8.8 Hz, 2H), 7.68 (d, J=8.8 Hz, 2H), 1.49 (s, 9H).

### Step 2: Synthesis of tert-butyl (4-aminophenyl) carbamate (3)

To a stirred solution of compound 2 (3 g, 1 eq) in ethyl acetate (50 mL), 10% Pd—C (0.3 g) was added and stirred at room temperature for 18 h under hydrogen atmosphere. The progress of the reaction was monitored by TLC. After completion of the reaction, the reaction mixture was concentrated under reduced pressure to afford the title compound 3 and used as such for the next step without further purification. LCMS (m/z): 109.05 (M−Boc).

### Step 3: Synthesis of tert-butyl (4-((6-(1-methyl-1H-benzo[d]imidazol-6-yl)-2-morpholinopyrimidin-4-yl) amino) phenyl) carbamate

The title compound was synthesized by following the general procedure described above for Buchwald coupling by using chloro compound 4 and amino compound 3. ¹H NMR (400 MHz, DMSO-d₆) δ: 9.23 (d, J=5.8 Hz, 2H), 8.26 (s, 1H), 8.17 (s, 1H), 7.86 (d, J=8.6 Hz, 1H), 7.71 (d, J=8.5 Hz, 1H), 7.54 (d, J=8.6 Hz, 2H), 7.39 (d, J=8.5 Hz, 2H), 6.62 (s, 1H), 3.91 (s, 3H), 3.86-3.68 (m, 8H), 1.48 (s, 9H); HPLC purity: 99%; LCMS Calculated for C₂₇H₃₁N₇O₃: 501.25; Observed: 502.25 (M+1).

### Step 4: Synthesis of N1-(6-(1-methyl-1H-benzo[d]imidazol-6-yl)-2-morpholinopyrimidin-4-yl)benzene-1,4-diamine

To a stirred solution of compound (3 g, 1 eq) in methanol (5 mL), methanolic HCl (3 mL) was added and stirred at room temperature for 18 h. The progress of the reaction was monitored by TLC. After completion of the reaction, the reaction mixture was concentrated under reduced pressure. The crude product was washed with THF, ethyl acetate and diethyl ether to afford title compound. ¹H NMR (400 MHz, DMSO-d₆) δ: 10.54 (s, 2H), 10.11 (s, 1H), 9.66 (s, 1H), 8.54-8.46 (m, 1H), 8.17 (dd, J=8.5, 1.5 Hz, 1H), 7.99 (d, J=8.6 Hz, 1H), 7.86-7.78 (m, 2H), 7.42-7.33 (m, 2H), 6.82 (s, 1H), 4.14 (s, 3H), 3.82-3.68 (m, 8H); HPLC purity: 98.52%; LCMS Calculated for C₂₂H₂₃N₇O (free base): 401.20; Observed: 402.25 (M+1).

### Examples 362-364

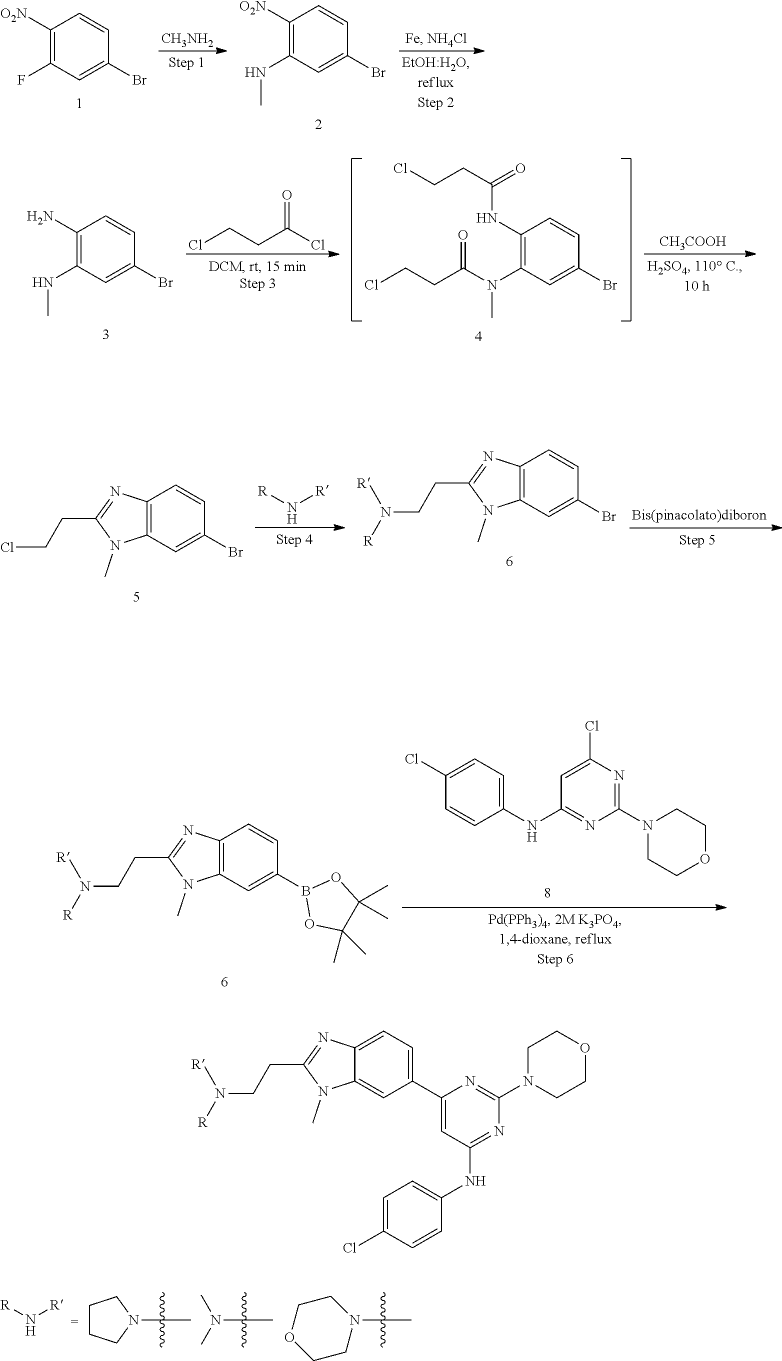

### Step 1: Synthesis of 5-bromo-N-methyl-2-nitroaniline (2)

To a stirred solution of the compound 1 (2 g, 1 eq)) in DMF (20 mL), methyl amine 2 M solution in THF (9.19 mL, 2 eq) and DIPEA (3.53 g, 3 eq) were added and stirred at room temperature for 2 h. The progress of the reaction was monitored by TLC. After completion of the reaction, the reaction mixture was diluted with water and extracted with ethyl acetate (3×25 mL). Combined organic extracts were washed with brine and dried over anhydrous sodium sulfate and evaporated under reduced pressure to afford the crude product 2 (2 g) and used as such for the next step without further purification.

### Step 2: Synthesis of 5-bromo-N1-methylbenzene-1,2-diamine (3)

To a stirred solution of compound 2 (6 g, 1 eq), in ethanol: water (2:1; 60 mL), iron powder (6 g) and ammonium chloride (5.53 g, 4 eq) were added and heated to reflux for 2 h. The progress of the reaction was monitored by TLC. After completion of the reaction, the reaction mixture was diluted with water and extracted with ethyl acetate (3×50 mL). Combined organic extracts were washed with brine and dried over anhydrous sodium sulfate and evaporated under reduced pressure to afford the crude compound 3. LCMS (m/z): 201.00 (M+1).

### Step 3: Synthesis of 6-bromo-2-(2-chloroethyl)-1-methyl-1H-benzo[d]imidazole (5)

To a stirred solution of compound 3 (2 g, 1 eq) in dichloromethane (20 mL), 3-chloropropanoyl chloride (1.08 g, 1 eq) was added at 0° C. and stirred for 15 min at same temperature. The reaction mixture was evaporated to dryness. The residue was dissolved in acetic acid (10 mL) and Conc.H₂SO₄ (1.5 mL) and heated to reflux for 10 h. The progress of the reaction was monitored by TLC. After completion of the reaction, the reaction mixture was evaporated under reduced pressure and basified to pH 8 using saturated sodium bicarbonate solution and extracted with ethyl acetate (3×50 mL). Combined organic extracts were washed with brine and dried over anhydrous sodium sulfate and evaporated under reduced pressure. The crude product was purified by column chromatography on silica gel 100-200 mesh using 30% EtOAc-hexane to afford the desired product 5 (2.05 g, impure). LCMS (m/z): 275.00 (M+2).

### Step 4: General procedure for the synthesis of compound 6

A stirred solution of compound 5 (1 eq) and corresponding amine (1 eq) in ethanol was heated to reflux for 18 h. The progress of the reaction was monitored by TLC. After completion of the reaction, the reaction mixture was evaporated to dryness. The residue was dissolved in water and extracted with 15% MeOH-DCM. Combined organic extracts were dried over anhydrous sodium sulfate and evaporated under reduced pressure to afford the crude compound 6.

| Structure | LCMS |
|---|---|
| 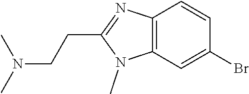 [SMILES: CN(C)CCc1nc2ccc(Br)cc2n1C] | LCMS (m/z): 282.10 (M + 1) |
| 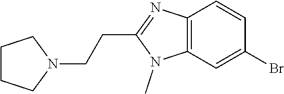 [SMILES: Cn1c(CCN2CCCC2)nc2ccc(Br)cc21] | LCMS (m/z): 308.15 (M + 1) |
| 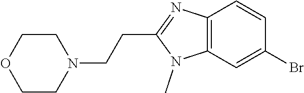 [SMILES: Cn1c(CCN2CCOCC2)nc2ccc(Br)cc21] | LCMS (m/z): 324.10 (M + 1) |

### Step 5: General Procedure for the Synthesis of Compound 7

The title compound 7 was synthesized by following the general procedure described above for Boronate ester preparation by using the respective bromo compound 6 and Bis(pinacolato)diboron.

| Structure | LCMS |
|---|---|
| 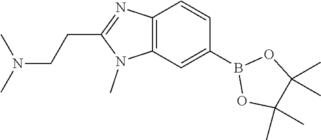 | LCMS (m/z): 330.20 (M + 1) |
| 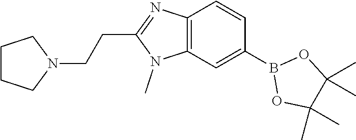 [SMILES: Cn1c(CCN2CCCC2)nc2ccc(B3OC(C)(C)C(C)(C)O3)cc21] | LCMS (m/z): 356.35 (M + 1) |
| 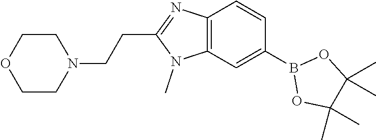 [SMILES: Cn1c(CCN2CCOCC2)nc2ccc(B3OC(C)(C)C(C)(C)O3)cc21] | LCMS (m/z): 372.15 (M + 1) |

### Step 6

The title compounds were synthesized by general procedure described above for Suzuki coupling by using the chloro compound 8 and the respective Boronate ester 7. The crude products were purified by preparative HPLC to afford the desired products.

N-(4-chlorophenyl)-6-(2-(2-(dimethylamino)ethyl)-1-methyl-1H-benzo[d]imidazol-6-yl)-2-morpholinopyrimidin-4-amine¹H NMR (400 MHz, CD₃OD) δ: 8.39 (s, 1H), 8.02-7.90 (m, 2H), 7.66 (d, J=8.1 Hz, 2H), 7.50-7.41 (m, 2H), 6.63 (s, 1H), 4.16 (s, 3H), 3.95-3.75 (m, 12H), 3.09 (s, 6H); HPLC purity: 97.01%; LCMS Calculated for C₂₆H₃₀ClN₇O (free base): 491.22; Observed: 492.25 (M+1).

N-(4-chlorophenyl)-6-(1-methyl-2-(2-(pyrrolidin-1-yl)ethyl)-1H-benzo[d]imidazol-6-yl)-2-morpholinopyrimidin-4-amine: ¹H NMR (400 MHz, CD3OD) δ: 8.30-8.25 (m, 1H), 7.96-7.82 (m, 2H), 7.65 (d, J=8.4 Hz, 2H), 7.49-7.41 (m, 2H), 6.61 (s, 1H), 4.09 (s, 3H), 3.94-3.81 (m, 10H), 3.68 (t, J=7.3 Hz, 2H), 3.56-3.50 (m, 4H), 2.22-2.18 (m, 4H); HPLC purity: 98.89%; LCMS Calculated for C₂₈H₃₂ClN₇O (free base): 517.24; Observed: 518.40 (M+1).

N-(4-chlorophenyl)-6-(1-methyl-2-(2-morpholinoethyl)-1H-benzo[d]imidazol-6-yl)-2-morpholinopyrimidin-4-amine: ¹H NMR (400 MHz, CD₃OD) δ: 8.40 (s, 1H), 8.03-7.91 (m, 2H), 7.66 (d, J=8.2 Hz, 2H), 7.49-7.42 (m, 2H), 6.62 (d, J=2.1 Hz, 1H), 4.18 (s, 3H), 4.08-4.04 (m, 4H), 3.95-3.79 (m, 12H), 3.58-3.50 (m, 4H); HPLC purity: 96.26%; LCMS Calculated for C₂₈H₃₂ClN₇O₂ (free base): 533.23; Observed: 534.00 (M+1).

### Example 365-366

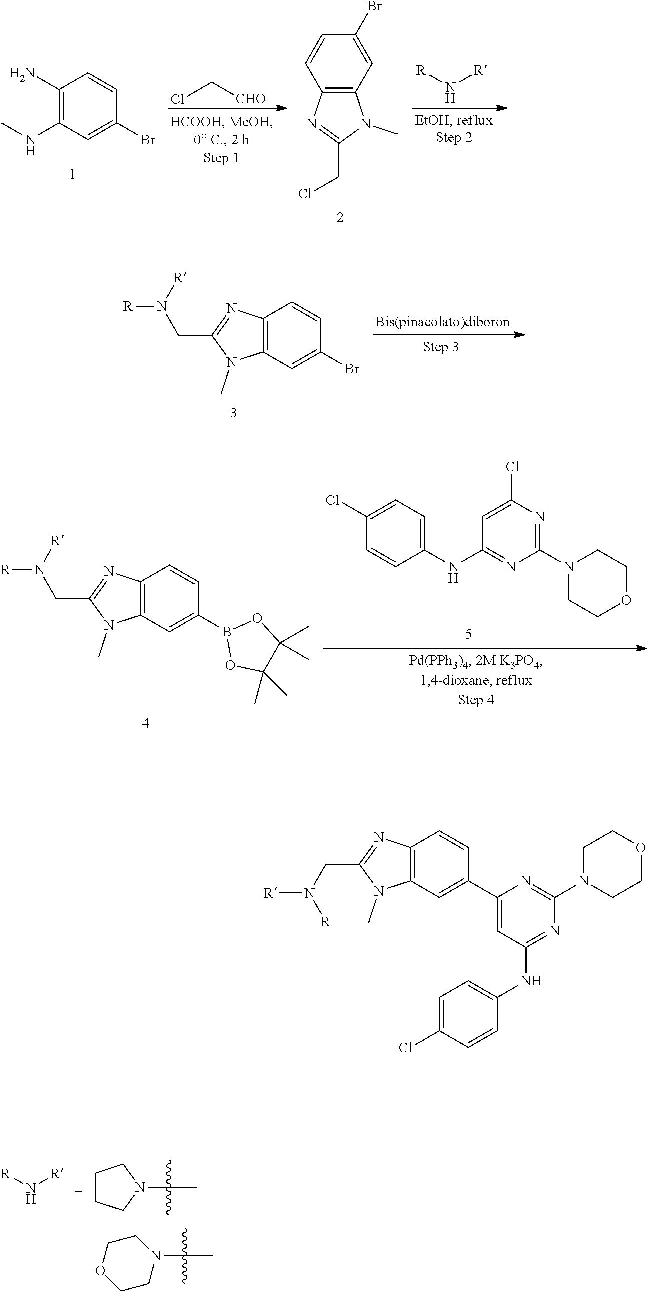

### Step 1: Synthesis of 6-bromo-2-(chloromethyl)-1-methyl-1H-benzo[d]imidazole

To a stirred solution of compound 1 (2 g, 1 eq) in methanol (20 mL), 2-chloroacetaldehyde (1.56 g, 2 eq) and formic acid (20 mL) were added at 0° C. and stirred at room temperature for 2 h. The progress of the reaction was monitored by TLC. After completion of the reaction, the reaction mixture was evaporated under reduced pressure. The crude product was washed with pentane and diethyl ether to afford the title compound 2. LCMS (m/z): 260.95 (M+2).

### Step 2: General Procedure for the Synthesis of Compound 3

A stirred solution of chloro compound 2 (1 eq) and corresponding amine (1 eq) in ethanol was heated to reflux for 4 h. The progress of the reaction was monitored by TLC. After completion of the reaction, the reaction mixture was evaporated to dryness. The residue was dissolved in water and extracted with 15% MeOH-DCM. Combined organic extracts were dried over anhydrous sodium sulfate and evaporated under reduced pressure to afford the crude compound 3.

| Structure | LCMS |
|---|---|
| 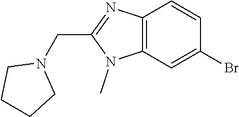 [SMILES: Cn1c(CN2CCCC2)nc2ccc(Br)cc21] | LCMS (m/z): 296.05 (M + 2) |
| 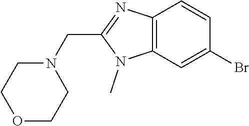 [SMILES: Cn1c(CN2CCOCC2)nc2ccc(Br)cc21] | LCMS (m/z): 312.10 (M + 2) |

### Step 3: General Procedure for the Synthesis of Compound 4

The title compound 4 was synthesized by following the general procedure described above for Boronate ester preparation by using the respective bromo compound 3 and Bis(pinacolato)diboron.

| Structure | LCMS |
|---|---|
| 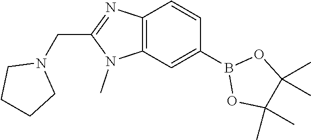 [SMILES: Cn1c(CN2CCCC2)nc2ccc(B3OC(C)(C)C(C)(C)O3)cc21] | LCMS (m/z): 342.30 (M + 1) |
| 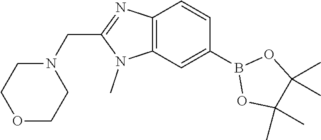 [SMILES: Cn1c(CN2CCOCC2)nc2ccc(B3OC(C)(C)C(C)(C)O3)cc21] | LCMS (m/z): 358.30 (M + 1) |

### Step 4

The title compounds were synthesized by the general procedure described above for Suzuki coupling by using the chloro compound 5 and the respective boronate ester 4. The crude product was purified by preparative HPLC to afford the desired product.

N-(4-chlorophenyl)-6-(1-methyl-2-(pyrrolidin-1-ylmethyl)-1H-benzo[d]imidazol-6-yl)-2-morpholinopyrimidin-4-amine¹H NMR (400 MHz, CDCl₃) δ: 8.03 (s, 1H), 7.83-7.67 (m, 2H), 7.41 (d, J=8.5 Hz, 2H), 7.33 (d, J=8.5 Hz, 2H), 6.54 (s, 1H), 6.49 (s, 1H), 3.97-3.77 (m, 13H), 2.62-2.54 (m, 4H), 1.82-1.76 (m 4H); HPLC purity: 97.24%; LCMS Calculated for C₂₇H₃₀ClN₇O: 503.22; Observed: 504.40 (M+1).

N-(4-chlorophenyl)-6-(1-methyl-2-(morpholinomethyl)-1H-benzo[d]imidazol-6-yl)-2-morpholinopyrimidin-4-amine: ¹H NMR (400 MHz, DMSO-d₆) δ: 9.51 (s, 1H), 8.14 (s, 1H), 7.86 (d, J=8.4 Hz, 1H), 7.68 (dd, J=24.1, 8.4 Hz, 3H), 7.36 (d, J=8.4 Hz, 2H), 6.66 (s, 1H), 3.92 (s, 3H), 3.79 (d, J=9.9 Hz, 6H), 3.75-3.68 (m, 4H), 3.58 (d, J=5.0 Hz, 4H), 2.44-2.40 (m, 4H); HPLC purity: 99.74%; LCMS Calculated for C₂₇H₃₀ClN₇O₂: 519.21; Observed: 520.40 (M+1).

### Example 367

### Synthesis of N-(4-chlorophenyl)-6-(1-methyl-2-(3-morpholinopropyl)-1H-benzo [d]imidazol-6-yl)-2-morpholinopyrimidin-4-amine

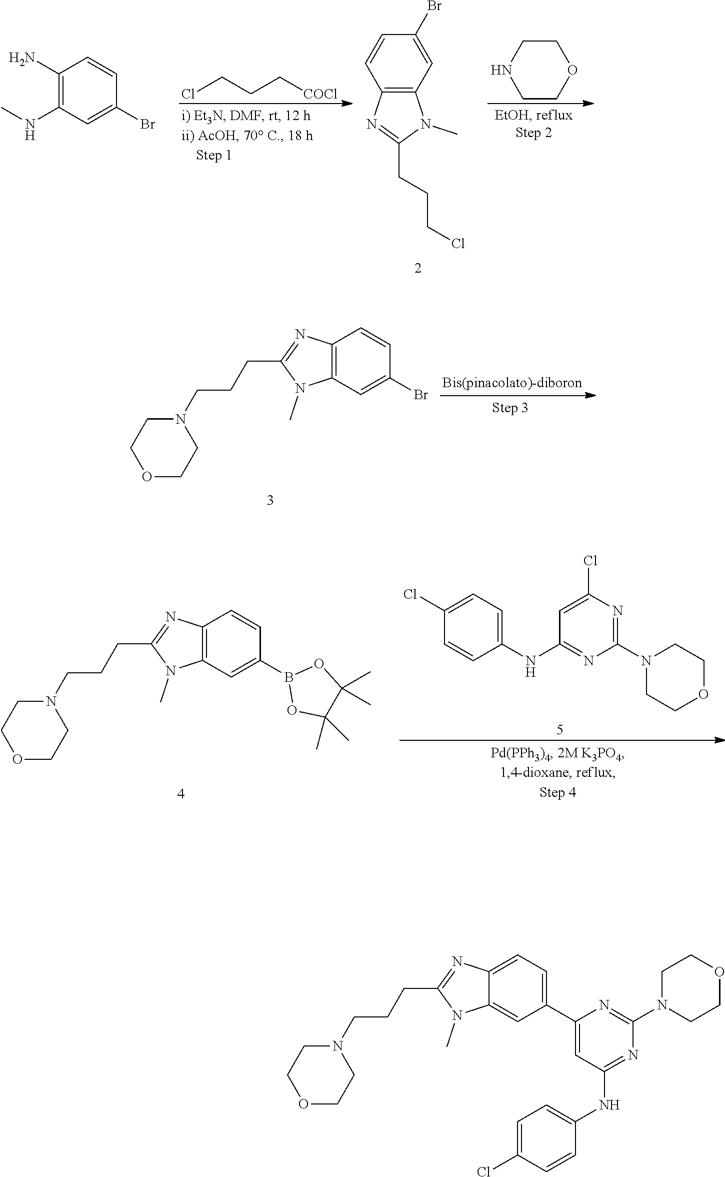

### Step 1: Synthesis of 6-bromo-2-(3-chloropropyl)-1-methyl-1H-benzo[d]imidazole (2

To a stirred solution of compound 1 (0.1 g, 1 eq) in DMF (5 mL),TEA (0.101 g, 2 eq) and 4-chlorobutanoyl chloride (0.061 g, 1 eq) was added at 0° C. and stirred at room temperature for 12 h. The progress of the reaction was monitored by TLC. After completion of the reaction, the reaction mixture was evaporated under reduced pressure and the residue was basified using saturated sodium bicarbonate solution and extracted with ethyl acetate (2×20 mL). Combined organic extracts were washed with brine and dried over anhydrous sodium sulfate and evaporated under reduced pressure. The crude product was dissolved in acetic acid (5 mL) and was heated to 70° C. for 18 h. The progress of the reaction was monitored by TLC. After completion of the reaction, the reaction mixture was evaporated under vacuum and residue was basified with saturated sodium bicarbonate solution and extracted with ethyl acetate (2×25 mL). Combined organic extracts were washed with brine and dried over anhydrous sodium sulfate and evaporated under reduced pressure. The crude product was purified by column chromatography on silica gel 100-200 mesh using 80% EtOAc-hexane to afford the title compound 2. LCMS (m/z): 289.00 (M+1).

### Step 2: Synthesis of 4-(3-(6-bromo-1-methyl-1H-benzo [d]imidazol-2-yl)propyl)morpholine (3)

A stirred solution of chloro compound 2 (0.1 g, 1 eq) and morpholine (0.121 g, 4 eq) in ethanol (5 mL) was heated to reflux for 18 h. The progress of the reaction was monitored by TLC. After completion of the reaction, the reaction mixture was evaporated to dryness. The residue was dissolved in water and extracted with 15% MeOH-DCM (2×25 mL). Combined organic extracts were dried over anhydrous sodium sulfate and evaporated under reduced pressure to afford the crude compound 3. LCMS (m/z): 340.15 (M+2).

### Step 3: Synthesis of 4-(3-(1-methyl-6-(4,4,5,5-tetramethyl-1,3,2-dioxaborolan-2-yl)-1H-benzo[d]imidazole-2-yl)propyl)morpholine (4)

The title compound was synthesized by following the general procedure described above for Boronate ester preparation by using bromo compound 3 and Bis(pinacolato)diboron.

### Step 4: Synthesis of N-(4-chlorophenyl)-6-(1-methyl-2-(3-morpholinopropyl)-1H-benzo[d]imidazol-6-yl)-2-morpholinopyrimidin-4-amine

The title compound was synthesized by the general procedure described above for Suzuki coupling by using the chloro compound 5 and boronate ester 4. The crude product was purified by preparative HPLC to afford the desired product. ¹H NMR (400 MHz, DMSO-d₆) δ: 9.49 (s, 1H), 8.11 (s, 1H), 7.83 (d, J=8.5 Hz, 1H), 7.71 (d, J=8.5 Hz, 2H), 7.60 (d, J=8.4 Hz, 1H), 7.36 (d, J=8.4 Hz, 2H), 6.65 (s, 1H), 3.84-3.69 (m, 11H), 3.57-3.45 (m, 4H), 2.92 (t, J=7.6 Hz, 2H), 2.37 (d, J=17.9 Hz, 6H), 2.00-1.90 (m, 2H); HPLC purity: 94.32%; LCMS Calculated for C₂₉H₃₄ClN₇O₂: 547.25; Observed: 548.35 (M+1).

### Example 368

### Synthesis of N-(4-chlorophenyl)-6-(2-(2-methoxyethyl)-1-methyl-1H-benzo[d]imidazol-6-yl)-2-morpholinopyrimidin-4-amine

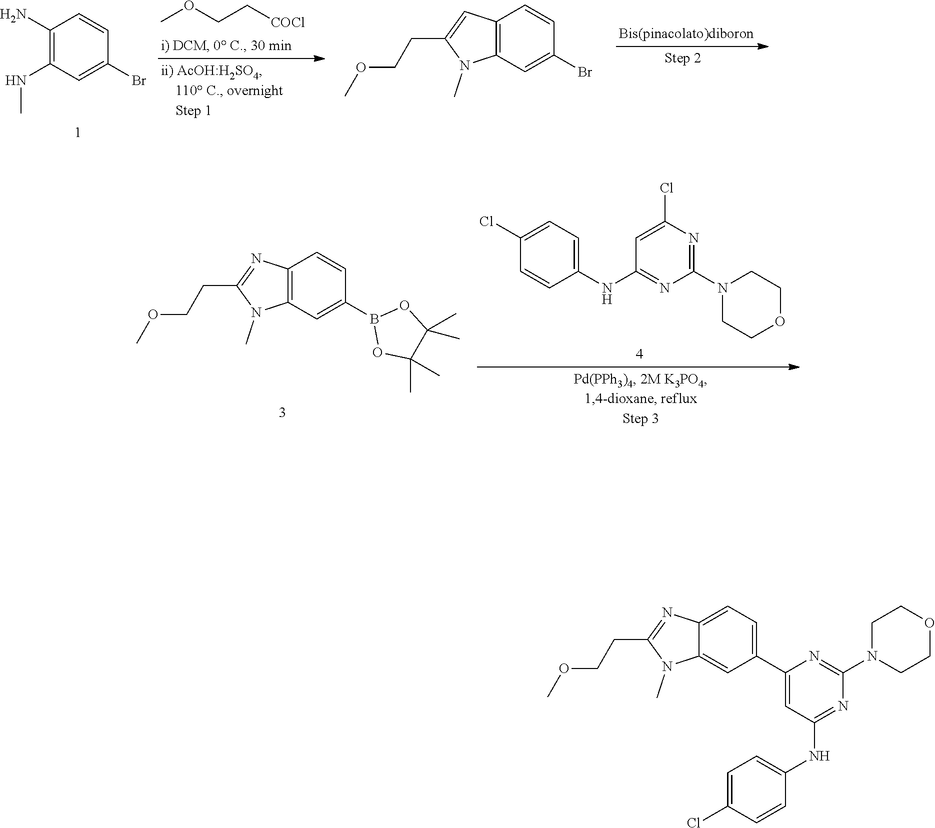

### Step 1: Synthesis of 6-bromo-2-(2-methoxyethyl)-1-methyl-1H-benzo[d]imidazole (2

To a stirred solution of compound 1 (0.7 g, 1 eq) in dichloromethane (5 mL), 3-methoxypropanoic acid (0.65 g, 1 eq) was added slowly at 0° C. and stirred at room temperature for 30 min. The reaction mixture was evaporated to dryness. The residue was dissolved in acetic acid (5 mL) and Conc.H₂SO₄ (1 mL) and heated to reflux for 18 h. The progress of the reaction was monitored by TLC. After completion of the reaction, the reaction mixture was evaporated under reduced pressure and basified to pH 8 using saturated sodium bicarbonate solution and extracted with ethyl acetate (2×25 mL). Combined organic extracts were washed with brine and dried over anhydrous sodium sulfate and evaporated under reduced pressure to afford the crude product 5 (0.81 g). LCMS (m/z): 269.00 (M+1).

### Step 2: Synthesis of 2-(2-methoxyethyl)-1-methyl-6-(4,4,5,5-tetramethyl-1,3,2-dioxaborolan-2-yl)-1H-benzo[d]imidazole (3

The title compound (crude) was synthesized by following the general procedure described above for Boronate ester preparation by using the bromo compound 2 and Bis(pinacolato)diboron. LCMS (m/z): 317.20 (M+1).

### Step 3: Synthesis of N-(4-chlorophenyl)-6-(2-(2-methoxyethyl)-1-methyl-1H-benzo[d]imidazol-6-yl)-2-morpholinopyrimidin-4-amine

The title compound has been synthesized by following the general procedure described above for Suzuki coupling by using compound 4 and Boronate ester 3. ¹H NMR (400 MHz, DMSO-d₆) δ: 9.50 (s, 1H), 8.12 (d, J=1.7 Hz, 1H), 7.84 (dd, J=8.5, 1.7 Hz, 1H), 7.76-7.67 (m, 2H), 7.62 (d, J=8.4 Hz, 1H), 7.41-7.32 (m, 2H), 6.66 (s, 1H), 3.85-3.72 (m, 13H), 3.29 (s, 3H), 3.16 (t, J=6.7 Hz, 2H); HPLC purity: 99.77%; LCMS Calculated for C₂₅H₂₇ClN₆O₂: 478.19; Observed: 479.25 (M+1).

### Examples 369-370

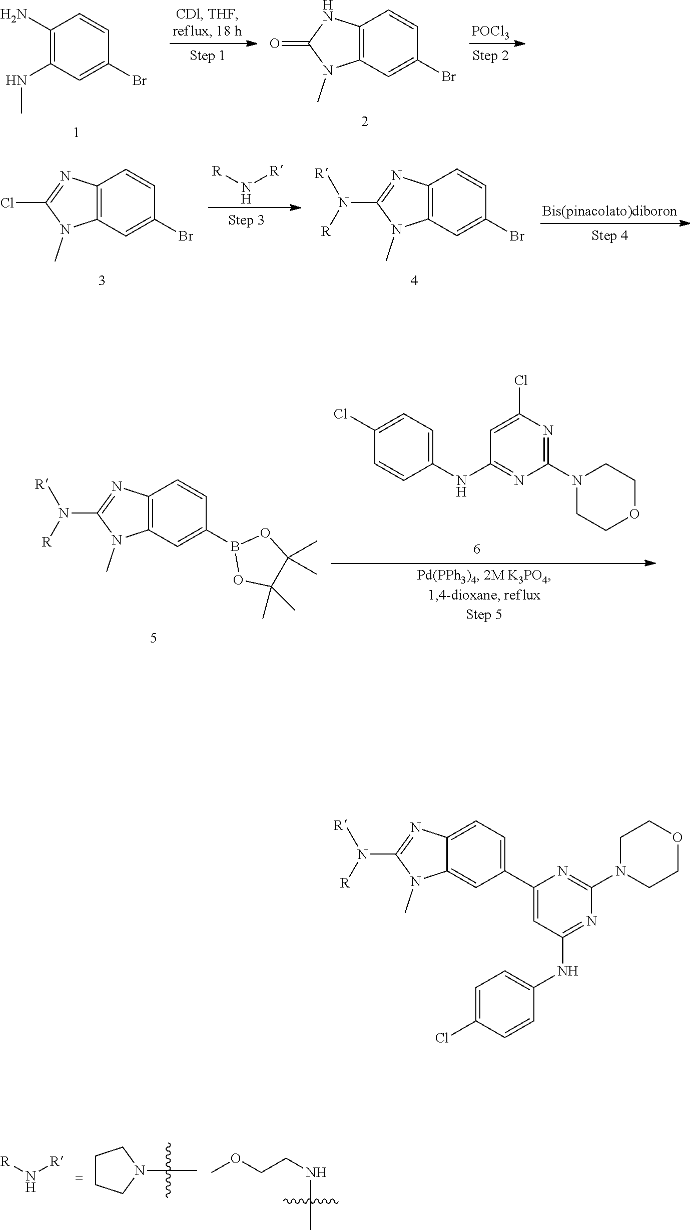

### Step 1: Synthesis of 6-bromo-1-methyl-1H-benzo[d]imidazol-2(3H)-one (2

To a stirred solution of compound 1 (2 g, 1 eq) in THF (40 mL), CDI (2.42 g, 1.5 eq) was added and heated to reflux for 18 h. The progress of the reaction was monitored by TLC. After completion of the reaction, the reaction mixture was evaporated under reduced pressure. The residue was diluted with ethyl acetate (100 mL) and washed with water. Organic layer was dried over anhydrous sodium sulfate and evaporated under reduced pressure to afford the crude compound 2. LCMS (m/z): 226.90 (M+1).

### Step 2: Synthesis of 6-bromo-2-chloro-1-methyl-1H-benzo[d]imidazole (3)

A stirred solution of compound 2 (0.1 g, 1 eq) in POCl₃ (3 mL) was heated at 110° C. for 18 h. The progress of the reaction was monitored by TLC. After completion of the reaction, the reaction mixture was slowly basified to pH 8 using saturated sodium bicarbonate solution and extracted with ethyl acetate (2×25 mL). Combined organic extracts were washed with brine and dried over anhydrous sodium sulfate and evaporated under reduced pressure. The crude product was purified by column chromatography on silica gel 100-200 mesh using 50% EtOAc-hexane to afford the title compound 3. LCMS (m/z): 244.95 (M+1).

### Step 3: General Procedure for the Synthesis of Compound 4

A stirred solution of compound 3 (1 eq) and corresponding amine (1 eq) in ethanol was heated to reflux for 18 h. The progress of the reaction was monitored by TLC. After completion of the reaction, the reaction mixture was evaporated to dryness. The residue was dissolved in water and extracted with 15% MeOH-DCM. Combined organic extracts were dried over anhydrous sodium sulfate and evaporated under reduced pressure to afford the crude compound 4.

| Structure | LCMS |
|---|---|
| 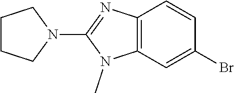 [SMILES: Cn1c(N2CCCC2)nc2ccc(Br)cc21] | LCMS (m/z): 280.05 (M + 1) |
| 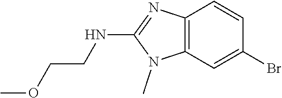 [SMILES: COCCNc1nc2ccc(Br)cc2n1C] | LCMS (m/z): 284.10 (M + 1) |

### Step 4: General Procedure for the Synthesis of Compound 5

The title compound 5 was synthesized by following the general procedure described above for Boronate ester preparation by using the respective bromo compound 4 and Bis(pinacolato)diboron.

| Structure | LCMS |
|---|---|
| 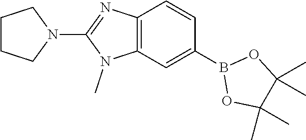 [SMILES: Cn1c(N2CCCC2)nc2ccc(B3OC(C)(C)C(C)(C)O3)cc21] | LCMS (m/z): 328.25 (M + 1) |
| 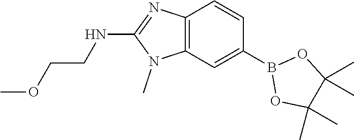 | LCMS (m/z): 332.25 (M + 1) |

### Step 5

The title compounds were synthesized by general procedure described above for Suzuki coupling by using the chloro compound 6 and the respective Boronate ester 5. The crude products were purified by preparative HPLC to afford the desired products.

N-(4-chlorophenyl)-6-(1-methyl-2-(pyrrolidin-1-yl)-1H-benzo[d]imidazol-6-yl)-2-morpholinopyrimidin-4-amine: ¹H NMR (400 MHz, DMSO-d₆) δ: 10.04-9.95 (m, 1H), 8.12 (s, 1H), 7.89 (d, J=8.4 Hz, 1H), 7.73 (d, J=8.7 Hz, 2H), 7.54 (d, J=8.3 Hz, 1H), 7.39 (d, J=8.6 Hz, 2H), 6.71 (s, 1H), 3.94 (s, 3H), 3.83-3.52 (m, 12H), 2.07-1.99 (m, 4H); HPLC purity: 96.29%; LCMS Calculated for C₂₆H₂₈ClN₇O (free base): 489.20; Observed: 490.35 (M+1).

6-(6-((4-chlorophenyl)amino)-2-morpholinopyrimidin-4-yl)-N-(2-methoxyethyl)-1-methyl-1H-benzo[d]imidazol-2-amine: ¹H NMR (400 MHz, DMSO-d₆) δ: 10.10 (s, 1H), 9.31 (t, J=5.5 Hz, 1H), 8.05 (s, 1H), 7.86 (d, J=8.3 Hz, 1H), 7.73 (d, J=8.6 Hz, 2H), 7.54 (d, J=8.2 Hz, 1H), 7.39 (d, J=8.6 Hz, 2H), 6.71 (s, 1H), 3.84-3.56 (m, 15H), 3.31 (s, 3H); HPLC purity: 95.66%; LCMS Calculated for C₂₅H₂₈ClN₇O₂ (free base): 493.20; Observed: 494.35 (M+1).

### Example 371

### Synthesis of 4-((2-morpholino-6-(1-(2-morpholinoethyl)-1H-benzo[d]imidazol-6-yl)pyrimidin-4-yl) amino)benzamide

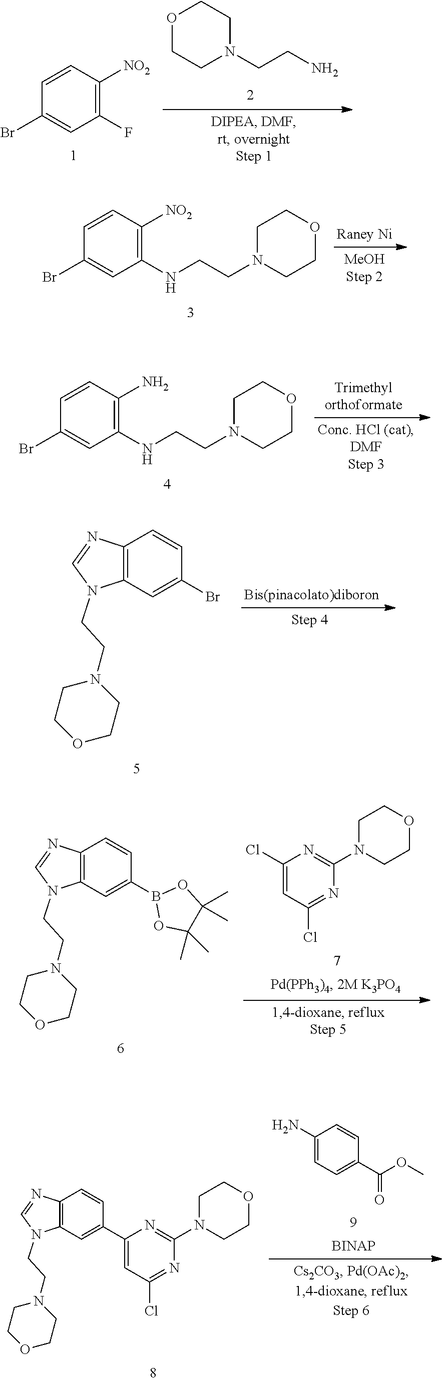

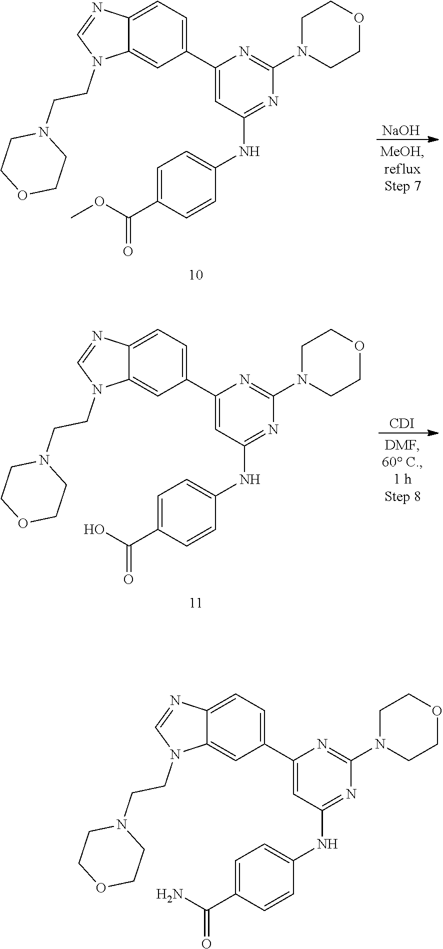

Step 1: Synthesis of 5-bromo-N-(2-morpholinoethyl)-2-nitroaniline (3)

To a stirred solution of 4-bromo-2-fluoro-1-nitrobenzene 1 (2 g, 1 eq), 2-morpholinoethanamine 2 (1.77 g, 1 eq) in DMF (20 mL), DIPEA (2.34 g, 2 eq) was added and stirred at room temperature for overnight. The progress of the reaction was monitored by TLC. After completion of the reaction, the reaction mixture was quenched with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 80% EtOAc-hexane to afford the title compound 3. LCMS (m/z): 330.05.90 (M+1).

### Step 2: Synthesis of 5-bromo-N1-(2-morpholinoethyl)benzene-1,2-diamine (4)

To a stirred solution of compound 3 (2.8 g, 1 eq) in methanol (30 mL), Raney nickel (3 g) was added and stirred at room temperature for 18 h under hydrogen atmosphere. The progress of the reaction was monitored by TLC. After completion of the reaction, the reaction mixture was filtered and evaporated under reduced pressure to afford the title compound 4. LCMS (m/z): 300.10 (M+1).

### Step 3: Synthesis of 4-(2-(6-bromo-1H-benzo[d]imidazol-1-yl)ethyl)morpholine (5)

To a stirred solution of compound 4 (2.3 g, 1 eq) in DMF (20 mL), trimethylorthoformate (2.43 g, 3 eq) and Conc. HCl (1 mL) were added and stirred at room temperature for 1 h. The progress of the reaction was monitored by TLC. After completion of the reaction, the reaction mixture was diluted with water and extracted with ethyl acetate (2×50 mL). Combined organic extracts were dried over anhydrous sodium sulfate and evaporated under reduced pressure. The crude product was purified by column chromatography on silica gel 100-200 mesh using 100% EtOAc to afford the title compound 5. LCMS (m/z): 310.10 (M+1).

### Step 4: Synthesis of 4-(2-(6-(4,4,5,5-tetramethyl-1,3,2-dioxaborolan-2-yl)-1H-benzo[d]imidazol-1-yl) ethyl)morpholine (6)

The title compound was synthesized by following the general procedure described above for Boronate ester preparation by using bromo compound 5and Bis (pinacolato)diboron. LCMS (m/z): 358.30 (M+1).

### Step 5: Synthesis of 4-(2-(6-(6-chloro-2-morpholinopyrimidin-4-yl)-1H-benzo[d]imidazol-1-yl)ethyl) morpholine (8)

The title compound was synthesized by general procedure described above for Suzuki coupling by using chloro compound 7 and Boronate ester 6. LCMS (m/z): 429.30 (M+1).

### Step 6: Synthesis of methyl 4-((2-morpholino-6-(1-(2-morpholinoethyl)-1H-benzo[d]imidazol-6-yl) pyrimidin-4-yl)amino)benzoate (10)

The title compound has been synthesized by following the general procedure described above for Buchwald Coupling by using chloro compound 8 and methyl 4-aminobenzoate 9. LCMS (m/z): 544.50 (M+1).

### Step 7: Synthesis of 4-((2-morpholino-6-(1-(2-morpholinoethyl)-1H-benzo[d]imidazol-6-yl)pyrimidin-4-yl)amino)benzoic acid (11)

The title compound has been synthesized by following the general procedure described above for ester hydrolysis by using compound 10 and sodium hydroxide. LCMS (m/z): 530.45 (M+1).

### Step 8: Synthesis of 4-((2-morpholino-6-(1-(2-morpholinoethyl)-1H-benzo[d]imidazol-6-yl) pyrimidin-4-yl) amino) benzamide

To a stirred solution of acid 11 (0.2 g, 1 eq) in DMF (3 mL) CDI (73 mg, 1.2 eq) was added and the resulting mixture stirred at 60° C. for 1 h. Reaction mixture was cooled to room temperature, ammonium hydroxide (0.132 g, 10 eq) was added and the reaction mixture was stirred for 1 h at room temperature. The progress of the reaction was monitored by TLC. After completion of the reaction, the reaction mixture was diluted with ethyl acetate (25 mL). Organic layer was washed with water, dried over anhydrous sodium sulfate and evaporated under reduced pressure. The crude product was purified by column chromatography on silica gel 100-200 mesh using 5% MeOH-DCM to afford the title compound. ¹H NMR (400 MHz, DMSO-d₆) δ: 8.59 (s, 1H), 8.09 (d, J=8.1 Hz, 1H), 7.95 (d, J=8.6 Hz, 1H), 7.91-7.82 (m, 3H), 7.81-7.74 (m, 2H), 7.18 (d, J=11.9 Hz, 1H), 6.80 (s, 1H), 5.03 (t, J=6.2 Hz, 2H), 3.98-3.74 (m, 18H), 3.18-3.10 (m, 2H); HPLC purity: 96.36%; LCMS Calculated for C₂₈H₃₂N₈O₃ (free base): 528.26; Observed: 529.45 (M+1).

### Example 372-373

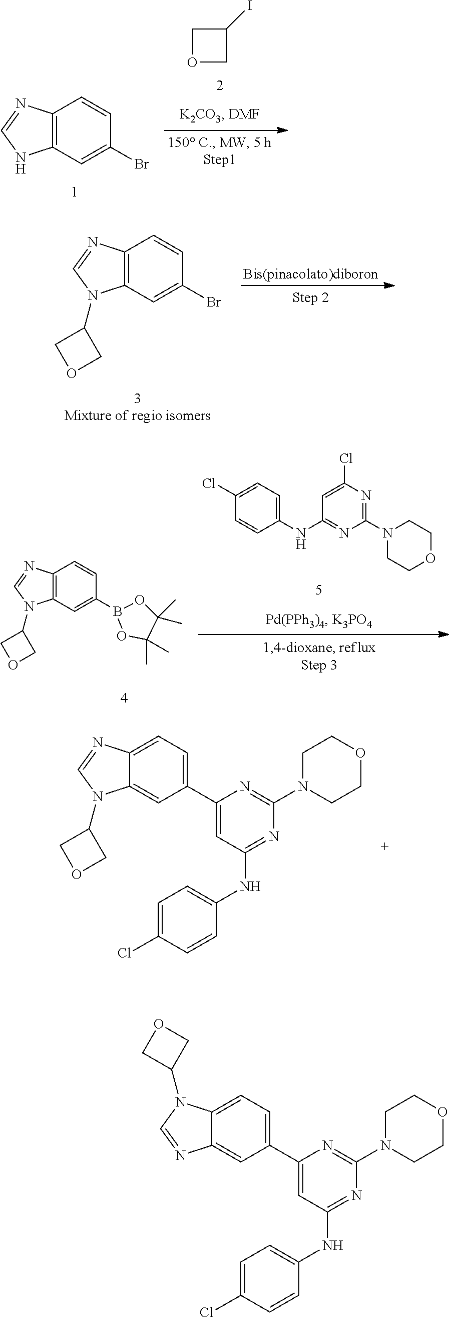

### Step 1: Synthesis of 6-bromo-1-(oxetan-3-yl)-1H-benzo[d]imidazole(3)

To a stirred solution of compound 1 (0.6 g, 1 eq) in DMF (2.5 mL), K₂CO₃ (0.855 g, 8 eq) and 3-iodooxetane 2 (0.73 g, 1.3 eq) were added and heated at 150° C. for 5 h in microwave. The progress of the reaction was monitored by TLC. After completion of the reaction, the reaction mixture was diluted with ethyl acetate (50 mL), washed with water, dried over anhydrous sodium sulfate and evaporated under reduced pressure to afford the title compound3. ¹H NMR confirms the formation of regioisomers. LCMS (m/z): 255.05 (M+2).

### Step 2: Synthesis of 1-(oxetan-3-yl)-6-(4,4,5,5-tetramethyl-1,3,2-dioxaborolan-2-yl)-1H-benzo[d] imidazole (4)

The title compound has been synthesized by following the general procedure described above for Boronate ester formation by using bromo compound 3 and Bis (pinacolato)diboron. LCMS (m/z): 301.15 (M+1).

### Step 3: Synthesis of N-(4-chlorophenyl)-2-morpholino-6-(1-(oxetan-3-yl)-1H-benzo[d]imidazol-6-yl) pyrimidin-4-amine

The title compound has been synthesized by following the general procedure described above for Suzuki coupling by using chloro compound 5 and boronate ester 4. Both the regioisomers were separated by chiral preparative HPLC. ¹H NMR (400 MHz, DMSO-d₆) δ: 9.57 (s, 1H), 8.58 (s, 1H), 8.43 (d, J=1.6 Hz, 1H), 7.94 (dd, J=8.5, 1.6 Hz, 1H), 7.82-7.67 (m, 3H), 7.41-7.32 (m, 2H), 6.66 (s, 1H), 5.85 (qd, J=7.4, 5.7 Hz, 1H), 5.18-5.01 (m, 4H), 3.83-3.68 (m, 8H); HPLC purity: 98.83%; LCMS Calculated for C₂₄H₂₃ClN₆O₂:462.16; Observed: 463.25 (M+1).

N-(4-chlorophenyl)-2-morpholino-6-(1-(oxetan-3-yl)-1H-benzo[d]imidazol-5-yl)pyrimidin-4-amine: ¹H NMR (400 MHz, DMSO-d₆) δ: 9.50 (s, 1H), 8.56 (s, 1H), 8.35 (d, J=1.7 Hz, 1H), 7.97 (dd, J=8.6, 1.7 Hz, 1H), 7.84 (d, J=8.6 Hz, 1H), 7.75-7.67 (m, 2H), 7.41-7.32 (m, 2H), 6.64 (s, 1H), 5.85-5.73 (m, 1H), 5.06 (dt, J=29.5, 7.1 Hz, 4H), 3.82-3.68 (m, 8H); HPLC purity: 99.79%; LCMS Calculated for C₂₄H₂₃ClN₆O₂:462.16; Observed: 463.25 (M+1).

N-(4-chlorophenyl)-6-(2-methyl-1-(oxetan-3-yl)-1H-benzo[d]imidazol-6-yl)-2-morpholinopyrimidin-4-amine: ¹H NMR (400 MHz, Chloroform-d) δ 8.71 (d, J=1.5 Hz, 1H), 7.89 (dd, J=8.5, 1.6 Hz, 1H), 7.73 (d, J=8.5 Hz, 1H), 7.46-7.38 (m, 2H), 7.35-7.27 (m, 2H), 6.52 (d, J=18.4 Hz, 2H), 5.61-5.50 (m, 1H), 5.33 (dd, J=7.5, 5.6 Hz, 2H), 5.24 (t, J=7.7 Hz, 2H), 3.93-3.85 (m, 4H), 3.80 (dd, J=5.5, 3.9 Hz, 4H), 2.61 (s, 3H), 1.26 (d, J=12.4 Hz, 1H); HPLC purity: 99.68%; LCMS Calculated for C₂₅H₂₅ClN₆O₂: 476.96:observed: 477.30(M+1).

### Example 374

### Synthesis of 6-(1-(cyclopropylmethyl)-2-methyl-1H-benzo[d]imidazol-6-yl)-N-(4-(5-methyl-4H-1,2,4-triazol-3-yl)phenyl)-2-morpholinopyrimidin-4-amine

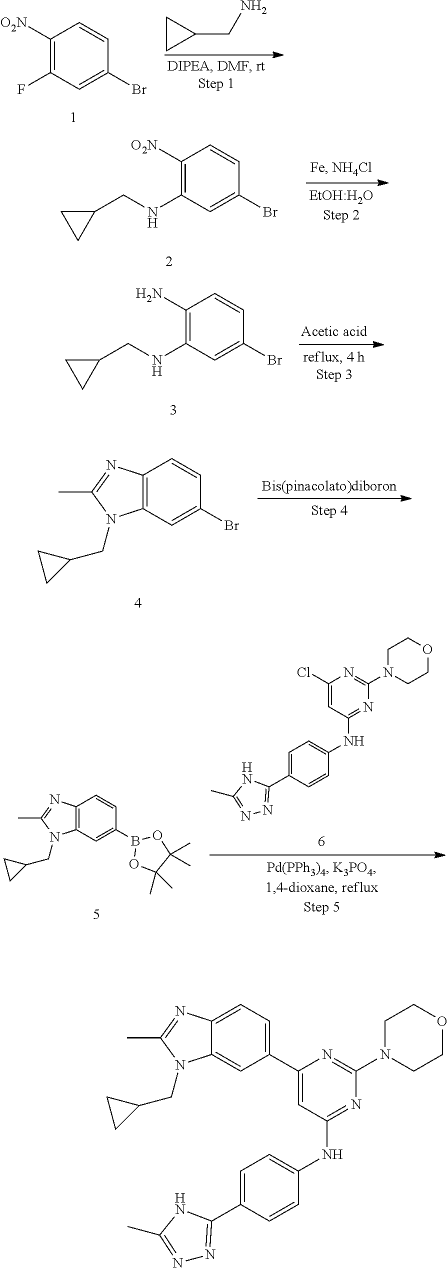

### Step 1: Synthesis of 5-bromo-N-(cyclopropylmethyl)-2-nitroaniline (2)

To a stirred solution of compound 1 (2 g, 1 eq) in DMF (20 mL), cyclopropylmethanamine (0.97 g,1.5 eq) and DIPEA (3.1 mL, 2 eq) were added at 0° C. and stirred at same temperature for 15 min. The reaction mixture was stirred at room temperature for 2 h. The progress of the reaction was monitored by TLC. After completion of the reaction, the reaction mixture was quenched with water and extracted with ethyl acetate (2×50 mL). Combined organic extracts were washed with brine, dried over anhydrous sodium sulfate and evaporated under reduced pressure to afford the title compound 2. LCMS (m/z): 272.90 (M+2).

### Step 2: Synthesis of 5-bromo-N1-(cyclopropylmethyl) benzene-1, 2-diamine (3)

The title compound has been synthesized by following the general procedure described above for reduction using the nitro compound 2 and Fe/NH₄Cl. LCMS (m/z): 241.00 (M+1).

### Step 3: Synthesis of 6-bromo-1-(cyclopropylmethyl)-2-methyl-1H-benzo[d]imidazole (4)

A stirred solution of compound 3 (1.85 g, 1 eq) in acetic acid (15 mL) was heated to reflux for 4 h. The progress of the reaction was monitored by TLC. After completion of the reaction, the reaction mixture was quenched with saturated sodium bicarbonate solution and extracted with ethyl acetate (2×50 mL). Combined organic extracts were washed with brine, dried over anhydrous sodium sulfate and evaporated under reduced pressure to afford the title compound 4. LCMS (m/z): 265.05 (M+1).

### Step 4: Synthesis of 1-(cyclopropylmethyl)-2-methyl-6-(4,4,5,5-tetramethyl-1,3,2-dioxaborolan-2-yl)-1H-benzo[d]imidazole (5)

The title compound (crude product) has been synthesized by following the general procedure described above for Boronate ester preparation by using bromo compound 4 and Bis (pinacolato)diboron. LCMS (m/z): 313.25 (M+1).

### Step 5: Synthesis of 6-(1-(cyclopropylmethyl)-2-methyl-1H-benzo[d]imidazol-6-yl)-N-(4-(5-methyl-4H-1,2,4-triazol-3-yl)phenyl)-2-morpholinopyrimidin-4-amine

The title compound has been synthesized by following the general procedure described above for Suzuki coupling by using chloro compound 6 and Boronate ester 5. ¹H NMR (400 MHz, DMSO-d₆) δ: 13.53 (s, 1H), 9.55 (s, 1H), 8.21 (s, 1H), 7.92 (d, J=8.4 Hz, 3H), 7.79 (s, 2H), 7.66 (d, J=8.5 Hz, 1H), 6.70 (s, 1H), 4.25 (d, J=6.9 Hz, 2H), 3.81-3.73 (m, 8H), 2.66 (s, 3H), 2.49 (s, 3H), 1.27 (td, J=12.4, 11.4, 6.4 Hz, 1H), 0.62-0.45 (m, 4H); HPLC purity: 98.55%; LCMS Calculated for C₂₉H₃₁N₉O: 521.27; Observed: 522.40 (M+1).

### Example 375

### Synthesis of 6-chloro-N-(4-(5-methyl-4H-1,2,4-triazol-3-yl)phenyl)-2-morpholinopyrimidin-4-amine

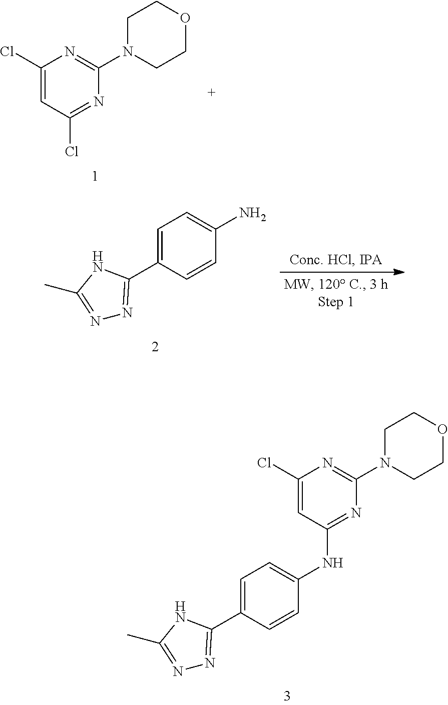 [SMILES: Clc1cc(Cl)nc(N2CCOCC2)n1 ; Cc1nnc(-c2ccc(N)cc2)[nH]1 ; Cc1nnc(-c2ccc(Nc3cc(Cl)nc(N4CCOCC4)n3)cc2)[nH]1]

### Step 1: Synthesis of 6-chloro-N-(4-(5-methyl-4H-1,2,4-triazol-3-yl)phenyl)-2-morpholinopyrimidin-4-amine (3)

To a stirred solution of compound 1 (0.6 g, 1 eq) in isopropanol (20 mL), compound 2 (0.803 g, 1 eq) and catalytic amount of concentrated HCl was added and stirred at 120° C. for 3 h in a microwave reactor. The progress of the reaction was monitored by TLC. After completion of the reaction, the reaction mixture was evaporated under reduced pressure. The residue was basified with saturated sodium bicarbonate and extracted with ethyl acetate (2×25 mL). Combined organic extracts were washed with brine, dried over anhydrous sodium sulfate and evaporated under reduced pressure. The crude product was purified by column chromatography on silica gel 100-200 mesh using 5% MeOH-DCM to afford the title compound3. LCMS (m/z): 372.20 (M+1).

### Example 376

### Synthesis of N-(4-chlorophenyl)-6-(2-cyclopropyl-1-methyl-1H-benzo [d] imidazol-6-yl)-2-morpholino pyrimidin-4-amine

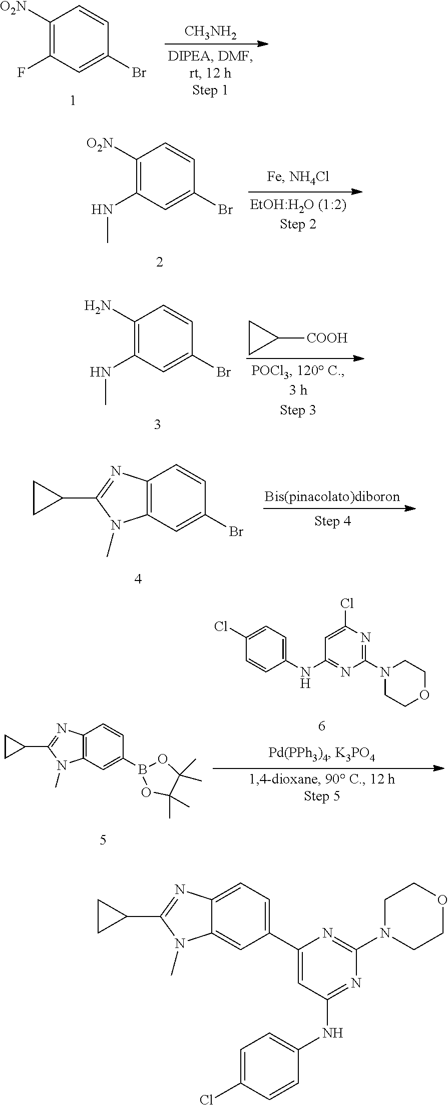

### Step 1: Synthesis of 5-bromo-N-methyl-2-nitroaniline (2)

To a stirred solution of compound 1 (10 g, 1 eq) in DMF (50 mL), DIPEA (17.59 g, 3 eq) and methylamine (3.52 g, 2.5 eq) were added and stirred at room temperature for 16 h. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure to afford the title compound 2.

### Step 2: Synthesis of 5-bromo-N1-methylbenzene-1,2-diamine (3)

To a stirred solution of compound 2 (8 g, 1 eq) in EtOH:H₂O (350 mL:150 mL), Iron powder (9.69 g, 5 eq) and NH₄Cl (9.26 g, 5 eq) were added. The resulting reaction mixture was refluxed for 3 h. The progress of the reaction was monitored by TLC. After completion of the reaction, the reaction mixture was filtered through a celite and the filtrate was evaporated under reduced pressure. The residue was diluted with water and extracted with ethyl acetate (3×50 mL). Combined organic extracts were washed with brine, dried over anhydrous sodium sulfate and evaporated under reduced pressure to afford the title compound 3. LCMS (m/z): 202.00 (M+1).

### Step 3: Synthesis of 6-bromo-2-cyclopropyl-1-methyl-1H-benzo[d]imidazole (4)

To a stirred solution of compound 3 (1 g, 1 eq) in POCl₃ (2 mL), cyclopropane carboxylic acid (0.5 mL) were added. The resulting reaction mixture was heated at 120° C. for 3 h. The progress of the reaction was monitored by TLC. After completion of the reaction, the reaction mixture was quenched with saturated sodium bicarbonate solution and extracted with ethyl acetate (3×20 mL). Combined organic extracts were washed with brine, dried over anhydrous sodium sulfate and evaporated under reduced pressure. The crude product was purified by column chromatography on silica gel 100-200 mesh using 20% EtOAc-hexane to afford the title compound 4. LCMS (m/z): 250.95 (M+1).

### Step 4: Synthesis of 2-cyclopropyl-1-methyl-6-(4,4,5,5-tetramethyl-1,3,2-dioxaborolan-2-yl)-1H-benzo[d]imidazole (5)

The title compound has been synthesized by following the general procedure described above for Boronate ester preparation by using bromo compound 4 and Bis (pinacolato)diboron. LCMS (m/z): 299.15 (M+1).

### Step 5: Synthesis of N-(4-chlorophenyl)-6-(2-cyclopropyl-1-methyl-1H-benzo[d]imidazol-6-yl)-2-morpholino pyrimidin-4-amine

The title compound has been synthesized by following the general procedure described above for Suzuki coupling by using chloro compound 6 and Boronate ester 5. HPLC purity: 99.43%; ¹NMR (400 MHz, DMSO-d₆) δ: 9.76 (s, 1H), 8.44 (s, 1H), 8.12 (d, J=8.6 Hz, 1H), 7.81 (d, J=8.6 Hz, 1H), 7.76-7.69 (m, 2H), 7.42-7.35 (m, 2H), 6.73 (s, 1H), 4.12 (s, 3H), 3.84-3.77 (m, 4H), 3.76-3.69 (m, 4H), 2.60 (dd, J=14.2, 7.6 Hz, 2H), 1.42 (t, J=5.3 Hz, 3H); LCMS Calculated for C₂₅H₂₅ClN₆O (free base): 460.18; Observed: 461.25 (M+1).

### Example 377-378

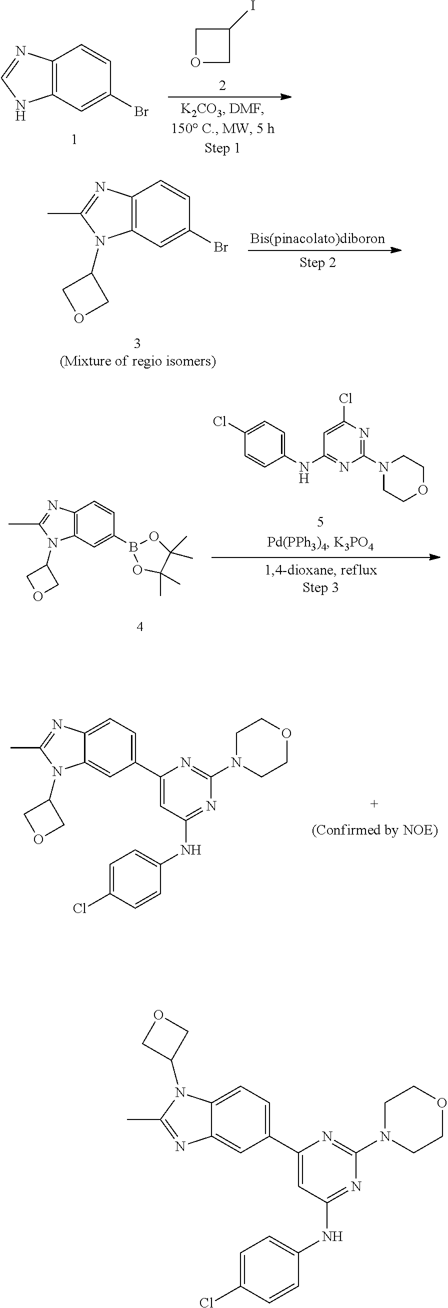

### Step 1: Synthesis of 6-bromo-2-methyl-1-(oxetan-3-yl)-1H-benzo[d]imidazole (3)

To a stirred solution of compound 1 (0.6 g, 1 eq) in DMF (6 mL), K₂CO₃ (0.784 g, 2 eq) and compound 2 (0.63 g, 1 eq) were added and heated at 180° C. for 5 h. The progress of the reaction was monitored by TLC. After completion of the reaction, the reaction mixture was diluted with ethyl acetate (50 mL), washed with water, dried over anhydrous sodium sulfate and evaporated under reduced pressure to afford the title compound3.¹H NMR confirms the formation of regioisomers. LCMS (m/z): 269.02 (M+2).

### Step 2: Synthesis of 2-methyl-1-(oxetan-3-yl)-6-(4,4,5,5-tetramethyl-1,3,2-dioxaborolan-2-yl)-1H-benzo[d]imidazole (4)

The title compound has been synthesized by following the general procedure described above for Boronate ester formation by using bromo compound 3 and Bis (pinacolato)diboron. LCMS (m/z): 315.05 (M+1).

### Step 3: Synthesis of N-(4-chlorophenyl)-6-(2-methyl-1-(oxetan-3-yl)-1H-benzo[d]imidazol-6-yl)-2-morpholinopyrimidin-4-amine and (4-chlorophenyl)[6-(2-methyl-1-oxetan-3-ylbenzimidazol-5-yl)-2-morpholin-4-ylpyrimidin-4-yl]amine

The title compounds have been synthesized by following the general procedure described above for Suzuki coupling by using chloro compound 5 and boronate ester 4. Both the regioisomers were separated by preparative HPLC.

N-(4-chlorophenyl)-6-(2-methyl-1-(oxetan-3-yl)-1H-benzo[d]imidazol-6-yl)-2-morpholinopyrimidin-4-amine: ¹H NMR (400 MHz, Chloroform-d) δ 8.71 (d, J=1.5 Hz, 1H), 7.89 (dd, J=8.5, 1.6 Hz, 1H), 7.73 (d, J=8.5 Hz, 1H), 7.46-7.38 (m, 2H), 7.35-7.27 (m, 2H), 6.52 (d, J=18.4 Hz, 2H), 5.61-5.50 (m, 1H), 5.33 (dd, J=7.5, 5.6 Hz, 2H), 5.24 (t, J=7.7 Hz, 2H), 3.93-3.85 (m, 4H), 3.80 (dd, J=5.5, 3.9 Hz, 4H), 2.61 (s, 3H); HPLC purity: 99.68%; LCMS Calculated for C₂₅H₂₅ClN₆O₂: 476.96; Observed: 477.30 (M+1). (4-chlorophenyl)[6-(2-methyl-1-oxetan-3-ylbenzimidazol-5-yl)-2-morpholin-4-ylpyrimidin-4-yl]amine: ¹H NMR (400 MHz, Chloroform-d) δ 8.33 (s, 1H), 8.06-7.95 (m, 2H), 7.43-7.25 (m, 4H), 6.55 (s, 1H), 6.48 (s, 1H), 5.52 (q, J=6.9 Hz, 1H), 5.33-5.17 (m, 4H), 3.89 (t, J=4.7 Hz, 4H), 3.80 (t, J=4.5 Hz, 4H), 2.59 (s, 3H); HPLC purity: 99.06%; LCMS Calculated for C₂₅H₂₅ClN₆O₂: 476.96; Observed: 477.25 (M+1).

### Example 379

### Synthesis of N-(6-(1-(cyclopropylmethyl)-2-methyl-1H-benzo[d]imidazol-6-yl)-2-morpholinopyrimidin-4-yl)-1-methyl-1H-indol-6-amine

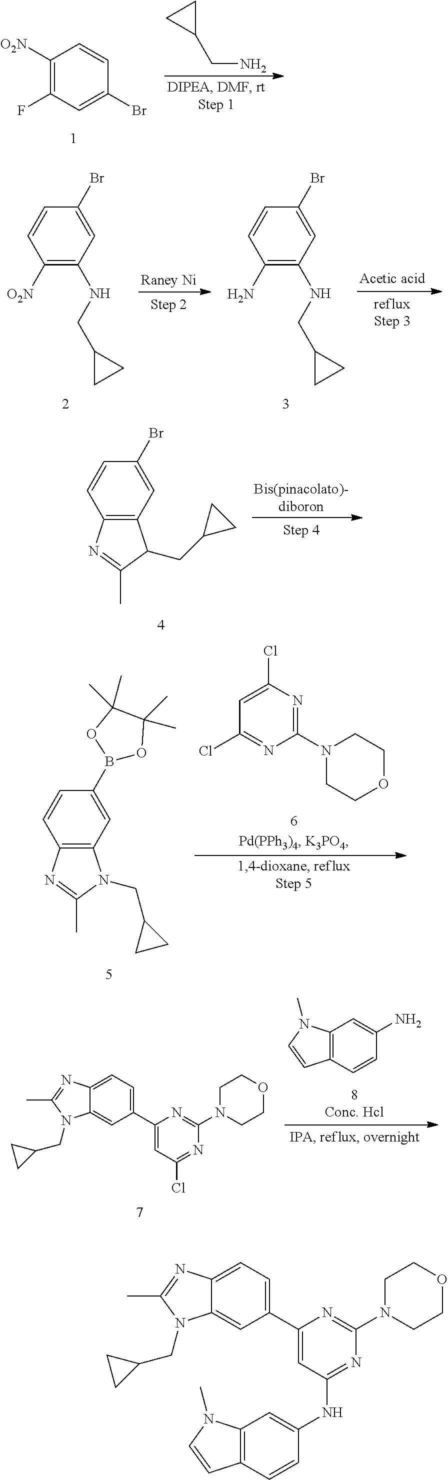

### Step-1:Synthesis of 5-bromo-N-(cyclopropylmethyl)-2-nitroaniline (2)

To a stirred solution of compound 1 (2 g, 1 eq) in DMF (10 mL), DIPEA (3.51 g, 3 eq) was added followed by the addition of cyclopropylmethanamine (0.774 g, 1.2 eq). The reaction mixture was stirred at rt for 16 h. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 15% EtOAc-hexane to afford the title compound 2. LCMS (m/z): 272.00 (M+2).

### Step-2: Synthesis of 5-bromo-N1-(cyclopropylmethyl) benzene-1, 2-diamine (3)

To a stirred solution of compound 2 (2.4 g, 1 eq) in methanol (30 mL), Raney nickel (2.4 g) was added under nitrogen atmosphere. The reaction mixture was stirred at room temperature for 18 h under hydrogen atmosphere (balloon pressure). The progress of the reaction was monitored by TLC. After completion of the reaction, the reaction mixture was filtered through celite and evaporated under reduced pressure to afford the title compound 3. LCMS (m/z): 267.05 (M+2).

### Step-3: Synthesis of6-bromo-1-(cyclopropylmethyl)-2-methyl-1H-benzo[d]imidazole(4)

To a stirred solution of compound 3 (2.1 g, 1 eq) in acetic acid (20 mL) and reaction mixture was heated at 120° C. for 3 h. The progress of the reaction was monitored by TLC. After completion of the reaction, the reaction mixture was basified to pH 7-8 with aq. Sodium carbonate and extracted with ethyl acetate (2×50 mL). Combined organic extracts were washed with brine, dried over anhydrous sodium sulfate and evaporated under reduced pressure. The crude product was purified by flash column chromatography to afford the title compound 4. LCMS (m/z): 267.00 (M+2).

### Step-4: Synthesis of1-(cyclopropylmethyl)-2-methyl-6-(4,4,5,5-tetramethyl-1,3,2-dioxaborolan-2-yl)-1H-benzo[d]imidazole (5)

The title compound has been synthesized by following the general procedure described above for Boronate ester preparation by using bromo compound 4 and Bis (pinacolato) diboron. LCMS (m/z): 313.00 (M+1).

### Step-5: Synthesis of4-(4-chloro-6-(1-(cyclopropylmethyl)-2-methyl-1H-benzo[d]imidazol-6-yl) pyrimidin-2-yl) morpholine (6)

The title compound has been synthesized by following the general procedure described above for Suzuki coupling by using chloro compound 6 and Boronate ester 5. LCMS (m/z): 384 (M+1).

### Step-6: Synthesis of N-(6-(1-(cyclopropylmethyl)-2-methyl-1H-benzo[d]imidazol-6-yl)-2-morpholinopyrimidin-4-yl)-1-methyl-1H-indol-6-amine

The title compound has been synthesized by following the general procedure described above for displacement reaction (IPA/HCl) by using chloro compound 7 and amine 8. ¹H NMR (400 MHz, DMSO-d₆) δ 9.38 (s, 1H), 8.21 (s, 1H), 8.16 (s, 1H), 7.84 (d, J=8.6 Hz, 1H), 7.63 (d, J=6.1 Hz, 1H), 7.50 (d, J=8.4 Hz, 1H), 7.28 (d, J=3.3 Hz, 1H), 7.08 (d, J=8.3 Hz, 1H), 6.66 (s, 1H), 6.35 (d, J=3.3 Hz, 1H), 4.18 (d, J=6.8 Hz, 2H), 3.90-3.78 (m, 4H), 3.75 (d, J=5.8 Hz, 7H), 2.57 (d, J=18.5 Hz, 3H), 1.25 (d, J=13.8 Hz, 1H), 0.59-0.52 (m, 2H), 0.47 (d, J=4.9 Hz, 2H); HPLC purity: 97.75%; LCMS Calculated for C₂₉H₃₁N₇O (free base): 493.60; Observed: 494.50 (M+1).

### Example 380

### Synthesis of 5-(6-((4-chlorophenyl)amino)-2-morpholinopyrimidin-4-yl)-1,3-dimethyl-1H-benzo[d]imidazol-2(3H)-one

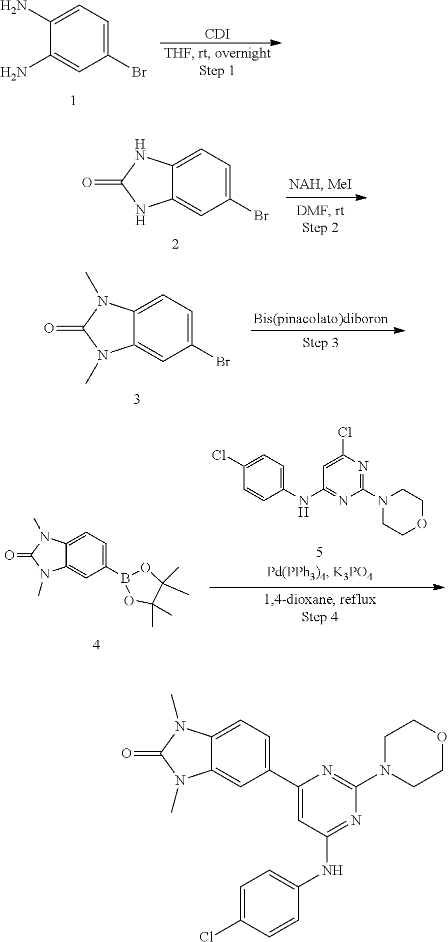

### Step-1; Synthesis of 5-bromo-1H-benzo[d]imidazol-2(3H)-one(2)

To a stirred solution of compound 1 (1 g, 1 eq) in dry THF (10 mL), CDI (0.953 g, 1.1 eq) was added under nitrogen atmosphere. The reaction mixture was stirred at room temperature for 16 h. The progress of the reaction was monitored by TLC. After completion of the reaction, the reaction mixture was evaporated under reduced pressure to obtain a crude residue. The crude product was purified by column chromatography on silica gel 100-200 mesh using 40% EtOAc-hexane to afford the title compounds 2. LCMS (m/z): 214.05 (M+1).

### Step-2: Synthesis of 5-bromo-1,3-dimethyl-1H-benzo[d]imidazol-2(3H)-one(3)

To a stirred solution of compound 2 (0.8 g, 1 eq) in DMF (10 mL), sodium hydride (0.48 g, 3.2 eq) was added at 0° C. and stirred for 30 min followed by the addition of methyl iodide (0.58 mL, 2.5 eq) at 0° C. and stirred at room temperature for 2 h. The progress of the reaction was monitored by TLC. After completion of the reaction, the reaction mixture was quenched with ice water and extracted with ethyl acetate (2×25 mL). Combined organic extracts were washed with brine, dried over anhydrous sodium sulfate and evaporated under reduced pressure to afford the title compound 3. LCMS (m/z): 242.20 (M+1).

### Step-3: Synthesis of 1,3-dimethyl-5-(4,4,5,5-tetramethyl-1,3,2-dioxaborolan-2-yl)-1H-benzo[d]imidazol-2(3H)-one(4)

The title compound have been synthesized by following the general procedure described above for Boronate ester preparation by using bromo compound 3 and Bis (pinacolato)diboron. LCMS (m/z): 289.05 (M+1).

### Step-4: Synthesis of Synthesis of5-(6-((4-chlorophenyl) amino)-2-morpholinopyrimidin-4-yl)-1, 3-dimethyl-1H-benzo [d]imidazol-2(3H)-one

The title compound has been synthesized by following the general procedure described above for Suzuki coupling by using chloro compound 5 and boronate ester 4 to afford title compound. The compound was taken in methanol (5 mL), methanol.HCl (0.2 mL) was added and stirred at rt for 30 min. the reaction mixture was evaporated under reduced pressure and purified by washing with ether to afford title compound as HCl salt. ¹H NMR (400 MHz, DMSO-d₆) δ 7.70 (dd, J=5.3, 3.5 Hz, 4H), 7.43-7.36 (m, 2H), 7.29 (d, J=8.1 Hz, 1H), 6.61 (s, 1H), 3.40 (d, J=11.1 Hz, 8H), 2.56 (s, 6H); HPLC purity: 995.59%; LCMS Calculated for C₂₃H₂₃ClN₆O₂ (free base): 450.92: Observed : 451.15(M+1).

### Example 381

### Synthesis of 5-(6-((4-chlorophenyl) amino)-2-morpholinopyrimidin-4-yl)-3-methyl-1-(2-morpholinoethyl)-1H-benzo[d]imidazol-2(3H)-one

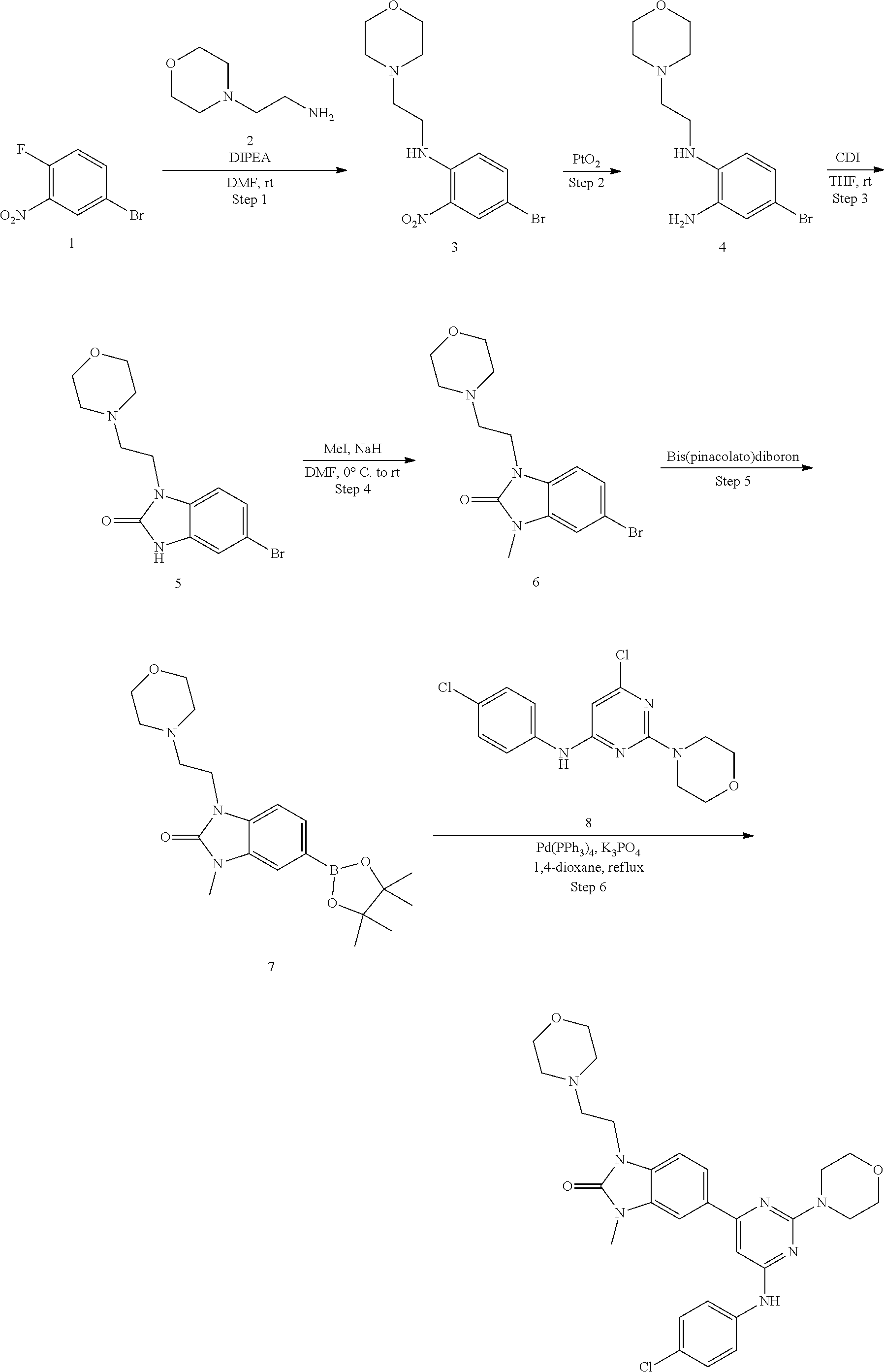

### Step-1: Synthesis of 4-bromo-N-(2-morpholinoethyl)-2-nitroaniline (3)

To a stirred solution of compound 1 (3.54 g, 1.5 eq) in dry DMF (20 mL), DIPEA (6.32 mL, 2 eq) was added followed by the addition of compound 2 (4 g, 1 eq). The reaction mixture was stirred at room temperature for 2 h. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure to afford title compound 3. LCMS (m/z): 332.05 (M+1).

### Step-2: Synthesis of 4-bromo-N1-(2-morpholinoethyl) benzene-1,2-diamine(4)

To a stirred solution of compound 3 (2.8 g, 1 eq) in methanol (50 mL), Raney Ni (2 g) was added under nitrogen atmosphere. The reaction mixture was stirred at room temperature for 16 h under hydrogen balloon pressure. The progress of the reaction was monitored by TLC. After completion of the reaction, the reaction mixture was filtered through celite and evaporated under reduced pressure to afford the title compound 4. LCMS (m/z): 302.1 (M+1).

### Step-3: Synthesis of 5-bromo-1-(2-morpholinoethyl)-1H-benzo[d]imidazol-2(3H)-one (5)

To a stirred solution of compound 4 (2.5 g, 1 eq) in dry THF (50 mL), CDI (1.48 g, 1.1 eq) was added under nitrogen atmosphere. The reaction mixture was stirred at room temperature for 16 h. The progress of the reaction was monitored by TLC. After completion of the reaction, the reaction mixture was evaporated under reduced pressure to obtain a crude residue. The crude product was purified by column chromatography on silica gel 100-200 mesh using 40% EtOAc-hexane to afford the title compounds 5. LCMS (m/z): 328 (M+1).

### Step-4: Synthesis of 5-bromo-3-methyl-1-(2-morpholinoethyl)-1H-benzo[d]imidazol-2(3H)-one(6)

To a stirred solution of compound 5 (0.5 g, 1 eq) in DMF (20 mL), sodium hydride (0.061 g, 1 eq) was added at 0° C. and stirred for 30 min followed by the addition of methyl iodide (0.1 mL, 1.1 eq) at 0° C. and stirred at room temperature for 2 h. The progress of the reaction was monitored by TLC. After completion of the reaction, the reaction mixture was quenched with ice water and extracted with ethyl acetate (2×25 mL). Combined organic extracts were washed with brine, dried over anhydrous sodium sulfate and evaporated under reduced pressure to afford the title compound 6. LCMS (m/z): 342 (M+1).

### Step-5: Synthesis of 3-methyl-1-(2-morpholinoethyl)-5-(4,4,5,5-tetramethyl-1,3,2-dioxaborolan-2-yl)-1H-benzo[d]imidazol-2(3H)-one(7)

The title compound have been synthesized by following the general procedure described above for Boronate ester preparation by using bromo compound 6 and Bis (pinacolato)diboron. LCMS (m/z): 342 (M+1).

### Step-6: Synthesis of 5-(6-((4-chlorophenyl) amino)-2-morpholinopyrimidin-4-yl)-3-methyl-1-(2-morpholinoethyl)-1H-benzo[d]imidazol-2(3H)-one

The title compound has been synthesized by following the general procedure described above for Suzuki coupling by using chloro compound 8 and boronate ester 7. ¹H NMR (400 MHz, DMSO-d₆) δ 9.14 (s, 1H), 7.85-7.60 (m, 4H), 7.40-7.23 (m, 3H), 6.62 (d, J=8.4 Hz,2H), 3.84-3.69 (m, 8H), 3.45 (s, 4H), 3.40 (s, 3H), 2.71-2.63 (m, 2H),2.23 (dd, J=5.3, 3.5 Hz, 4H); HPLC purity: 98.74%; LCMS Calculated for C₂₈H₃₂ClN₇O₃ (free base): 550.05:observed: 450.45(M+1).

Synthesis of 6-(1H-benzo[d]imidazol-4-yl)-N-(4-chlorophenyl)-2-morpholinopyrimidin-4-amine

The title compound has been synthesized by following the general procedure described above for Suzuki coupling by using chloro compound 1 and Boronic acid. ¹H NMR (400 MHz, Chloroform-d) δ 8.14 (s, 1H), 7.85 (d, J=8.0 Hz, 1H), 7.70 (d, J=7.6 Hz, 1H), 7.42 (d, J=8.4 Hz, 2H), 7.32 (dt, J=7.9, 3.6 Hz, 3H), 6.62 (s, 1H), 3.84 (s, 9H), 3.54 (s, 1H); HPLC purity: 99.68%; LCMS Calculated for C₂₁H₁₉ClN₆O (free base): 406.87:observed: 407.20 (M+1).

### Example 382

### Synthesis of 6-(6-((4-chlorophenyl)amino)-2-morpholinopyrimidin-4-yl)-1-methyl-1,3-dihydro-2H-benzo[d]imidazol-2-one

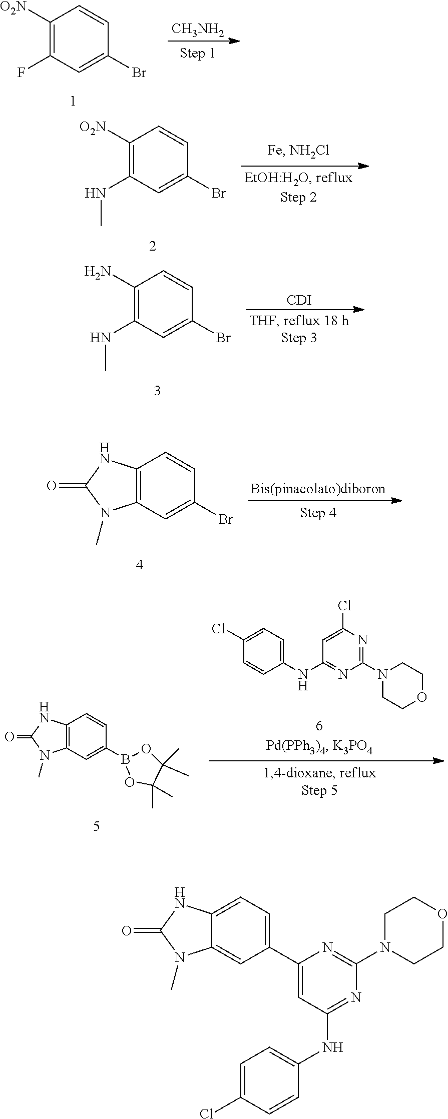

### Step 1: Synthesis of 5-bromo-N-methyl-2-nitroaniline (2)

To a stirred solution of compound 1 (2 g, 1 eq) in DMF (20 mL), 2 M methyl amine solution in THF (9.13 mL, 2 eq) and DIPEA (3.53 g, 2.5 eq) were added and stirred at room temperature for 2 h. The progress of the reaction was monitored by TLC. After completion of the reaction, the reaction mixture was diluted with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 20% EtOAc-hexane to afford the title compound 2. LCMS (m/z): 231.05 (M+1).

### Step 2: Synthesis of 5-bromo-N1-methylbenzene-1,2-diamine (3

To a stirred solution of compound 2 (1 g, 1 eq) in ethanol (40 mL), iron powder (2.31 g, 5 eq), water (20 mL) and ammonium chloride (2.18 g, 5 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate (100 mL), washed with water, brine, dried over anhydrous sodium sulfate and evaporated under reduced pressure to afford the title compound 3. LCMS (m/z): 200.95 (M+1).

### Step 3: Synthesis of 6-bromo-1-methyl-1,3-dihydro-2H-benzo[d]imidazol-2-one (4)

To a stirred solution of compound 3 (2 g, 1 eq) in THF (40 mL), CDI (2.42g, 1.5 eq) was added slowly at room temperature. The reaction mixture was heated to reflux for 18 h. The progress of the reaction was monitored by TLC. After completion of the reaction, the reaction mixture was evaporated under reduced pressure. The residue was diluted with water and extracted with ethyl acetate (2×50 mL). Combined organic extracts were washed with brine, dried over anhydrous sodium sulfate and evaporated under reduced pressure to afford the title compound 4. LCMS (m/z): 226.90 (M+1).

### Step 4: Synthesis of 1-methyl-6-(4,4,5,5-tetramethyl-1,3,2-dioxaborolan-2-yl)-1,3-dihydro-2H-benzo[d]imidazol-2-one (5)

The title compound has been synthesized by following the general procedure described above for Boronate ester preparation by using bromo compound 4 and Bis (pinacolato)diboron. LCMS (m/z): 275.20 (M+1).

### Step 5: Synthesis of 6-(6-((4-chlorophenyl)amino)-2-morpholinopyrimidin-4-yl)-1-methyl-1,3-dihydro-2H-benzo[d]imidazol-2-one

The title compound has been synthesized by following the general procedure described above for Suzuki coupling by using chloro compound 6 and boronate ester 5. ¹H NMR (400 MHz, DMSO-d₆) δ: 11.21 (s, 1H),10.20 (s, 1H), 7.71 (d, J=8.5 Hz, 2H), 7.60 (d, J=17.0 Hz, 2H), 7.41 (d, J=8.5 Hz, 2H), 7.13 (d, J=8.1 Hz, 1H), 6.62 (s, 1H), 3.82-3.72 (m, 8H),3.36 (s, 3H); HPLC purity: 99.74%; LCMS Calculated for C₂₂H₂₁ClN₆O₂ (free base):436.14; Observed: 437.35 (M+1).

### Example 383

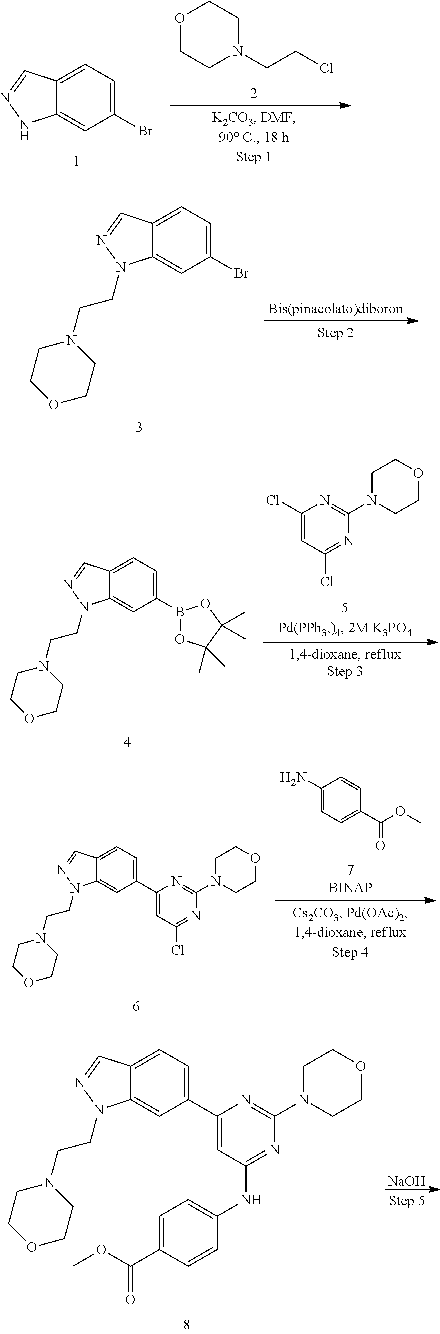

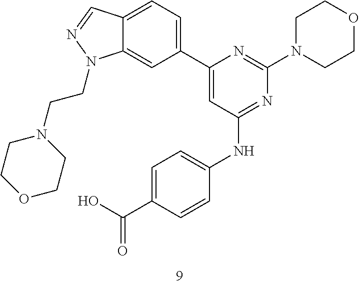 [SMILES: O=C(O)c1ccc(Nc2cc(-c3ccc4cnn(CCN5CCOCC5)c4c3)nc(N3CCOCC3)n2)cc1]

### Step 1: Synthesis of 4-(2-(6-bromo-1H-indazol-1-yl)ethyl)morpholine (3)

To a stirred solution of 6-bromo indazole 1 (3 g, 1 eq) in DMF (20 mL), K₂CO₃ (6.3 g, 3 eq) and 4-(2-chloroethyl)morpholine (3.4 g, 1.2 eq) were added and heated at 90° C. for 18 h. The progress of the reaction was monitored by TLC. After completion of the reaction, the reaction mixture was diluted with water and extracted with ethyl acetate (3×50 mL). Combined organic extracts were washed with water, dried over anhydrous sodium sulfate and evaporated under reduced pressure. The crude product was purified by column chromatography on silica gel 100-200 mesh using 40% EtOAc-hexane to afford the title compound 3. LCMS (m/z): 310.05 (M+1).

### Step 2: Synthesis of 4-(2-(6-(4,4,5,5-tetramethyl-1,3,2-dioxaborolan-2-yl)-1H-indazol-1-yl)ethyl) morpholine (4)

The title compound (crude) was synthesized by following the general procedure described above for Boronate ester preparation by using bromo compound 3 and Bis(pinacolato)diboron.

### Step 3: Synthesis of 4-(2-(6-(6-chloro-2-morpholinopyrimidin-4-yl)-1H-indazol-1-yl)ethyl)morpholine (6)

The title compound was synthesized by general procedure described above for Suzuki coupling by using chloro compound 5 and Boronate ester 4. LCMS (m/z): 429.30 (M+1).

### Step 4: Synthesis of methyl 4-((2-morpholino-6-(1-(2-morpholinoethyl)-1H-indazol-6-yl)pyrimidin-4-yl) amino)benzoate (8)

The title compound has been synthesized by following the general procedure described above for Buchwald Coupling by using chloro compound 6 and methyl 4-aminobenzoate 7. LCMS (m/z): 544.45 (M+1).

### Step 5: Synthesis of 4-((2-morpholino-6-(1-(2-morpholinoethyl)-1H-indazol-6-yl) pyrimidin-4-yl) amino) benzoic acid

The title compound has been synthesized by following the general procedure described above for ester hydrolysis by using compound 5 and sodium hydroxide. ¹H NMR (400 MHz, DMSO-d₆) δ: 12.58 (s, 1H), 9.81 (s, 1H), 8.29 (s, 1H), 8.12 (s, 1H), 7.92 (d, J=8.4 Hz, 2H), 7.90-7.73 (m, 4H), 6.77 (s, 1H), 4.62 (t, J=6.6 Hz, 2H), 3.83-3.75 (m, 8H), 3.48 (t, J=4.5 Hz, 4H), 2.79 (t, J=6.5 Hz, 2H), 2.44 (t, J=4.7 Hz, 4H); HPLC purity: 99.14%; LCMS Calculated for C₂₈H₃₁N₇O₄: 529.24; Observed: 530.40 (M+1).

### Example 384

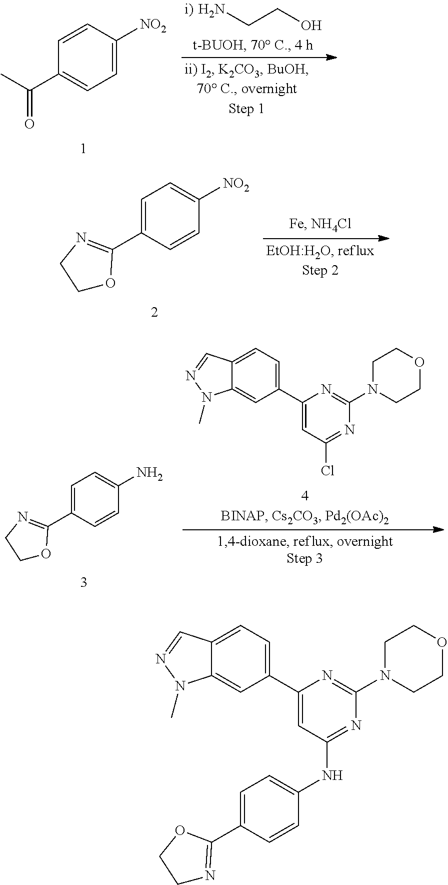

### Step 1: General Procedure for Cyclization

To a stirred solution of compound 1 (1 eq) in t-butanol, 2-aminoethanol (1.1 eq) was added and stirred at 70° C. for 4 h followed by the addition of K₂CO₃ (3 eq) and Iodine (1.2 eq). The reaction mixture was heated at 70° C. for overnight. The progress of the reaction was monitored by TLC. After completion of the reaction, the reaction mixture was evaporated to dryness. The residue was diluted with sodium thiosulphate and extracted with ethyl acetate. Combined organic extracts were washed with water, brine, dried over anhydrous sodium sulfate and evaporated under reduced pressure. The crude product was purified by column chromatography on silica gel to afford compound 2 2-(4-nitrophenyl)-4,5-dihydrooxazole. LCMS: (m/z): 192.95 (M+1)

### Step 2: General Procedure for Reduction

To a stirred solution of compound 2 (1 eq) in ethanol, iron powder (4 eq), water and ammonium chloride (4 eq) were added slowly. The reaction mixture was heated to reflux for 2 h. The progress of the reaction was monitored by TLC. After completion of the reaction, the reaction mixture was filtered through celite and evaporated under reduced pressure. The residue was dissolved in ethyl acetate and washed with brine, dried over anhydrous sodium sulfate and evaporated under reduced pressure to afford the title compound 3, 4-(4,5-dihydrooxazol-2-yl)aniline. LCMS: (m/z): 163.05 (M+1).

### Step 3: Synthesis of N-(4-(4,5-dihydrooxazol-2-yl)phenyl)-6-(1-methyl-111-indazol-6-yl)-2-morpholinopyrimidin-4-amine

The title compound has been synthesized by following the general procedure described above for Buchwald coupling by using chloro compound 4 and compound 3. ¹H NMR (400 MHz, DMSO-d₆) δ: 10.09 (s, 1H), 8.63 (t, J=5.5 Hz, 1H), 8.24 (s, 1H), 8.12 (d, J=2.7 Hz, 1H), 7.92-7.77 (m, 5H), 7.72 (d, J=8.5 Hz, 1H), 6.80 (s, 1H), 4.14 (s, 3H), 3.88-3.69 (m, 10H), 3.58 (q, J=6.1 Hz, 2H); HPLC purity: 97.52%; LCMS Calculated for C₂₅H₂₅N₇O₂ (free base): 455.21; Observed: 456.30 (M+1).

### Example 385

### Synthesis of N-(4-(1H-pyrrol-3-yl)phenyl)-6-(1-methyl-1H-indazol-6-yl)-2-morpholinopyrimidin-4-amine

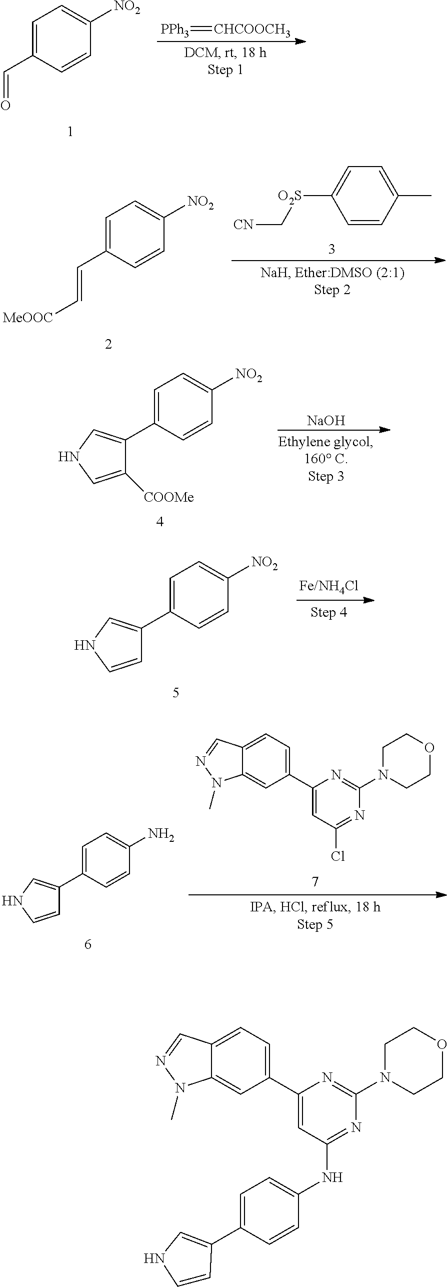

### Step 1: Synthesis of (E)-methyl 3-(4-nitrophenyl)acrylate (2)

To a stirred solution of compound 1 (1 g, 1 eq) in dichloromethane (50 mL), PPh₃═CHCOOCH₃ (3.32 g, 1 eq) was added and stirred for 18 hat room temperature. The progress of the reaction was monitored by TLC. After completion of the reaction, the reaction mixture was diluted with water and extracted with dichloromethane (3×25 mL). Combined organic extracts were washed with brine, dried over anhydrous sodium sulfate and evaporated under reduced pressure. The crude product was purified by column chromatography on silica gel 100-200 mesh using 10% EtOAc-hexane to afford the title compound 2.

### Step 2: Synthesis of methyl 4-(4-nitrophenyl)-1H-pyrrole-3-carboxylate (4)

To a stirred solution of NaH (0.12 g, 1.3 eq) in diethyl ether (7 mL), a solution of compound 2 (0.8 g, 1 eq) and compound 3 (0.792 g, 1.05 eq) in DMSO: diethyl ether(1:1.3; 23 mL) was added drop wise and stirred for 4 h at room temperature. The progress of the reaction was monitored by TLC. After completion of the reaction, the reaction mixture was diluted with water and extracted with ethyl acetate (2×25 mL). Combined organic extracts were washed with brine, dried over anhydrous sodium sulfate and evaporated under reduced pressure. The crude product was purified by column chromatography on silica gel 100-200 mesh using 40% EtOAc-hexane to afford title compound 4.41 NMR (400 MHz, DMSO-d₆) δ: 11.82 (s, 1H), 8.17 (d, J=8.8 Hz, 2H), 7.75 (d, J=8.8 Hz, 2H), 7.58 (t, J=2.4 Hz, 1H), 7.22 (t, J=2.4 Hz, 1H), 3.67 (s, 3H).

### Step 3: Synthesis of 3-(4-nitrophenyl)-1H-pyrrole (5)

To a stirred solution of compound 4 (0.3 g, 1 eq) in ethylene glycol (5 mL), NaOH (0.121 g, 2.5 eq) was added and stirred at 160° C. in a sealed tube for 5 h. The progress of the reaction was monitored by TLC. After completion of the reaction, the reaction mixture was diluted with water and extracted with ethyl acetate (2×25 mL). Combined organic extracts were washed with brine, dried over anhydrous sodium sulfate and evaporated under reduced pressure. The crude product was purified by column chromatography on silica gel 100-200 mesh using 30% EtOAc-hexane to afford the title compound 5. ¹H NMR (400 MHz, DMSO-d₆) δ: 11.26 (s, 1H), 8.14 (d, J=9.2 Hz, 2H), 7.78 (d, J=8.8 Hz, 2H), 7.52 (s, 1H), 6.88 (s, 1H), 6.60 (s, 1H).

### Step 4: Synthesis of 4-(1H-pyrrol-3-yl) aniline (6)

To a stirred solution of compound 5 (1 eq) in EtOH:H₂O (1:1, 30 mL), Iron powder (4.01 eq) and NH₄Cl (4.01 eq) were added. The resulting reaction mixture was refluxed for 3 h. The progress of the reaction was monitored by TLC. After completion of the reaction, the reaction mixture was filtered through a celite and the filtrate was evaporated under reduced pressure. The residue was diluted with water and extracted with ethyl acetate (3×50 mL). Combined organic extracts were washed with brine, dried over anhydrous sodium sulfate and evaporated under reduced pressure to afford the title compound. LCMS (m/z): 158.95 (M+1).

### Step 5: Synthesis of N-(4-(1H-pyrrol-3-yl)phenyl)-6-(1-methyl-1H-indazol-6-yl)-2-morpholinopyrimidin-4-amine

The title compound has been synthesized by following the general procedure described above for displacement reaction (IPA, Conc. HCl) by using chloro compound 7 and amine 6. ¹H NMR (400 MHz, DMSO-d₆) δ: 10.85 (s, 1H), 9.36 (s, 1H), 8.23 (s, 1H), 8.09 (s, 1H), 7.84 (d, J=8.5 Hz, 1H), 7.79-7.72 (m, 1H), 7.62 (d, J=8.3 Hz, 2H), 7.49 (d, J=8.6 Hz, 2H), 7.16 (d, J=2.2 Hz, 1H), 6.78 (q, J=2.4 Hz, 1H), 6.70 (s, 1H), 6.41 (t, J=2.2 Hz, 1H), 4.13 (s, 3H), 3.86-3.73 (m, 8H); HPLC purity: 90.57%; LCMS Calculated for C₂₆H₂₅N₇O: 451.21; Observed: 452.30 (M+1).

### Example 386

### Synthesis of N-(4-chlorophenyl)-6-(5-fluoro-l-methyl-3-(2-morpholinoethyl)-1H-indazol-6-yl)-2-morpholinopyrimidin-4-amine

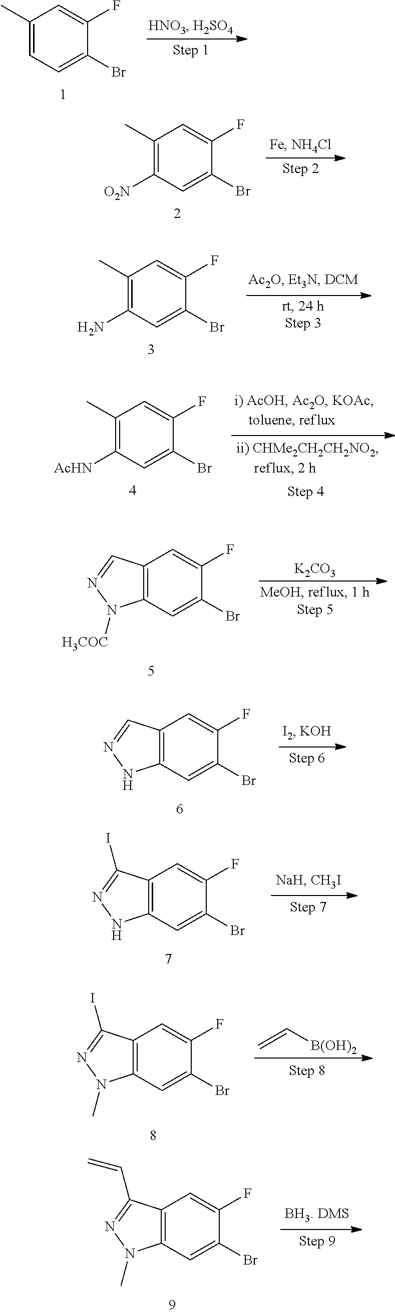

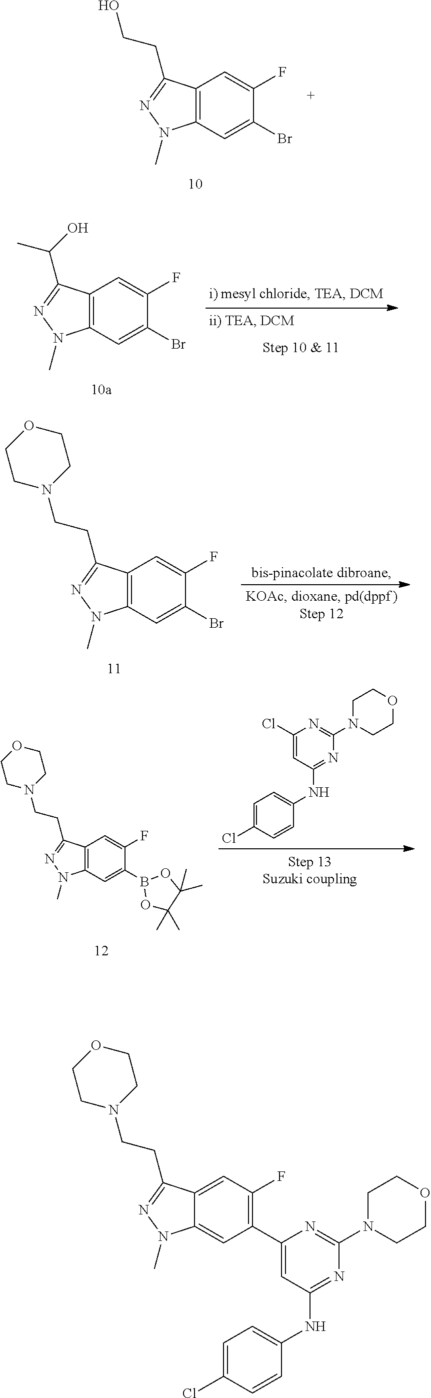 [SMILES: Cn1nc(CCO)c2cc(F)c(Br)cc21 ; CC(O)c1nn(C)c2cc(Br)c(F)cc12 ; Cn1nc(CCN2CCOCC2)c2cc(F)c(Br)cc21 ; Cn1nc(CCN2CCOCC2)c2cc(F)c(B3OC(C)(C)C(C)(C)O3)cc21 ; Clc1ccc(Nc2cc(Cl)nc(N3CCOCC3)n2)cc1 ; Cn1nc(CCN2CCOCC2)c2cc(F)c(-c3cc(Nc4ccc(Cl)cc4)nc(N4CCOCC4)n3)cc21]

### Step-1: Synthesis of 1-bromo-2-fluoro-4-methyl-5-nitrobenzene (2)

To a stirred solution of compound 1 (8 g, 1 eq) in Conc. H₂SO₄ (35 mL), nitrating mixture (13.2 mL Conc. HNO₃+5 mL Conc. H₂SO₄) was added at 0° C. and stirred at same temperature for 2 h. The progress of the reaction was monitored by TLC. After completion of the reaction, the reaction mixture was poured onto ice water and basified to pH 9 using 2N NaOH. The precipitated solid was filtered, washed with water and dried under reduced pressure to afford the title compound 2. LCMS (m/z): 235 (M+1).

### Step-2: Synthesis of 5-bromo-4-fluoro-2-methylaniline (3)

To a stirred solution of compound 2 (7.2 g, 1 eq) in EtOH: H₂O (7:3, 100 mL), Iron powder (8.1 g, 5 eq) and NH₄Cl (8.5 g, 5 eq) were added. The resulting reaction mixture was refluxed for 3 h. The progress of the reaction was monitored by TLC. After completion of the reaction, the reaction mixture was filtered through a celite and the filtrate was evaporated under reduced pressure. The residue was diluted with water and extracted with ethyl acetate (3×50 mL). Combined organic extracts were washed with brine, dried over anhydrous sodium sulfate and evaporated under reduced pressure to afford the title compound. LCMS (m/z): 205.15 (M+1).

### Step-3: Synthesis of N-(5-bromo-4-fluoro-2-methylphenyl)acetamide(4)

To a stirred solution of compound 3 (5.5 g, 1 eq) in DCM (50 mL), acetic anhydride (3.8 mL, 1.5 eq), and TEA (8.9 mL, 2.4 eq) was added and stirred under nitrogen atmosphere at room temperature for 18 h. The reaction mixture was evaporated under reduced pressure. Residue was diluted with methanol and potassium carbonate was added, reaction was stirred at rt for 1 h. The reaction mixture was evaporated under reduced pressure and diluted with water, precipitate solid was filtered and dried to afford title compound. LCMS (m/z): 247.15 (M+1).

### Step-4: Synthesis of 1-(6-bromo-5-fluoro-1H-indazol-1-yl)ethan-1-one(5)

To a stirred solution of compound 4 (5.5 g, 1 eq) in toluene (50 mL) and glacial acetic acid (6.4 mL), acetic anhydride (6.3 mL, 3 eq) and potassium acetate (4.8g, 2.2 eq). The reaction mixture was stirred warm at refluxed, then added isopentyl nitrite(3.5 mL, 1.2 eq)and stirred for 3 h. The reaction mixture was concentrated under reduced pressure to obtain a crude compound.

### Step-5: Synthesis of 6-bromo-5-fluoro-1H-indazole(6)

To a stirred solution of compound 5 (4g, 1 eq) in methanol (50 mL), potassium carbonate (9.8 g, 3.2 eq) was added and heated to reflux for 1 h. The reaction mixture was evaporated under reduced pressure to obtain a residue. The residue was diluted with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 30% EtOAc-hexane to afford the title compound. LCMS (m/z): 216 (M+2).

### Step-6: Synthesis of 6-bromo-5-fluoro-3-iodo-1H-indazole(7)

To a stirred solution of compound 6 (1.7 g, 1 eq) in dioxane (30 mL), iodine (4 g, 2 eq) and 3NaOH (10 mL) was added and stirred for 2 h. The progress of the reaction was monitored by TLC. After completion of the reaction, the reaction mixture was quenched with water and extracted with ethyl acetate (3×50 mL). Combined organic extracts were washed with sodium bicarbonate solution and sodium thiosulfate solution, dried over anhydrous sodium sulfate and evaporated under reduced pressure. LCMS (m/z): 343.15 (M+2).

### Step-7: Synthesis of 6-bromo-5-fluoro-3-iodo-1-methyl-1H-indazole (8)

To a stirred solution of compound 7 (2 g, 1 eq) in dry DMF (20 mL), NaH (0.388 g, 1.5 eq) was added at 0° C. and stirred for 15 min followed by the addition of methyl iodide (0.8 mL, 2 eq). The reaction mixture was stirred at room temperature for 30 min. The progress of the reaction was monitored by TLC. After completion of the reaction, the reaction mixture was quenched with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 20% EtOAc-hexane to afford the title compound. LCMS (m/z): 356.09 (M+2).

### Step-8: Synthesis of 6-bromo-5-fluoro-1-methyl-3-vinyl-1H-indazole (9)

The title compound has been synthesized by following the general procedure described above for Suzuki coupling by using bromo compound 8 and boronic acid. LCMS (m/z): 256.1 (M+1).

### Step-9: Synthesis of 2-(6-bromo-5-fluoro-1-methyl-1H-indazol-3-yl)ethan-1-ol(10)

To a stirred solution of compound 9 (0.48 g, 1 eq) in dry THF (30 mL), BH3:DMS (6.2 mL, 8 eq) was added at 0° C. and stirred at room temperature for 16 h. The progress of the reaction was monitored by TLC. After completion of the reaction, the reaction mixture was quenched with 4N NaOH and 30% H₂O₂ solution at 0° C. The reaction mixture was stirred at room temperature for 3 h and extracted with ethyl acetate (3×50 mL). Combined organic extracts were dried over anhydrous sodium sulfate and evaporated under reduced pressure. The crude product was purified by column chromatography on silica gel 100-200 mesh using 50% EtOAc-hexane to afford the title compound. LCMS (m/z): 273 (M+1).

### Step-10 & 11: Synthesis of 4-(2-(6-bromo-5-fluoro-1-methyl-1H-indazol-3-yl)ethyl)morpholine (11)

To a stirred solution of compound 10 (0.4 g, 1 eq) in DCM (5 mL), TEA (0.4 mL, 2 eq) was added and stirred for 15 min followed by the slow addition of mesyl chloride (0.17 mL, 1.5 eq) at 0° C. The reaction mixture was stirred at rt for 2 h. The progress of the reaction was monitored by TLC. After completion of the reaction, the reaction mixture was diluted with water, extracted with DCM (3×25 mL). The combined organic extracts were washed with brine, dried over anhydrous sodium sulfate and evaporated under reduced pressure to crude residue. The residue was dissolved in DMF(3 mL),triethylamine(0.27 mL) was added and stirred at 90° C. for 2 h. The progress of the reaction was monitored by TLC. After completion of the reaction, the reaction mixture was quenched with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 30% EtOAc-hexane to afford the compound. LCMS (m/z): 273 (M+1).

### Step-12: Synthesis of4-(2-(5-fluoro-1-methyl-6-(4,4,5,5-tetramethyl-1,3,2-dioxaborolan-2-yl)-1H-indazol-3-yl)ethyl)morpholine(12)

The title compound has been synthesized by following the general procedure described above for boronate ester coupling by using bromo compound 11 and Boronate ester. LCMS (m/z): 390.25 (M+1).

### Step-13: Synthesis of Synthesis of N-(4-chlorophenyl)-6-(5-fluoro-1-methyl-3-(2-morpholinoethyl)-1H-indazol-6-yl)-2-morpholinopyrimidin-4-amine

The title compound has been synthesized by following the general procedure described above for Suzuki coupling by using chloro compound 13 and Boronate ester compound 12. ¹H NMR (400 MHz, DMSO-d₆) δ 9.64 (s, 1H), 8.23 (s, 1H), 7.75 (d, J=8.6 Hz, 3H), 7.39 (d, J=9 Hz, 2H), 6.73 (s, 1H), 4.04 (s, 3H), 3.82-3.65 (m, 8H),3.40-3.35(m,4H), 3.08(t, J=2.6 Hz, 2H),2.78 (t, J=2.4 Hz, 2H); HPLC purity: 96.31%; LCMS Calculated for C₂₈H₃₁ClFN₇O₂: 552.05; Observed: 552.45 (M+1).

### Example 387

### Synthesis of N-(4-(4-methyl-2H-1,2,3-triazol-2-yl)phenyl)-6-(1-methyl-3-(2-morpholinoethyl)-1H-indazol-6-yl)-2-morpholinopyrimidin-4-amine

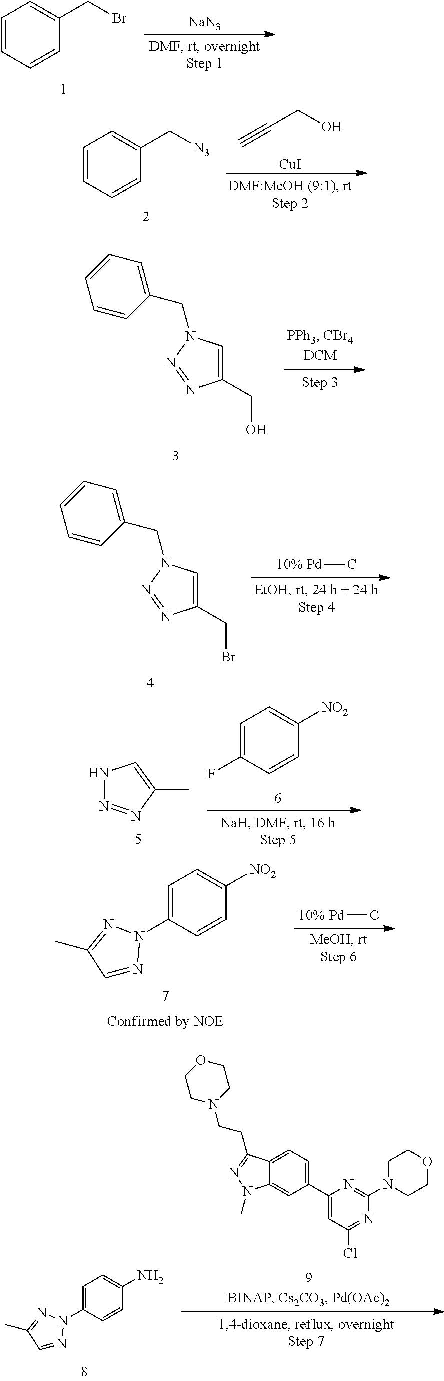

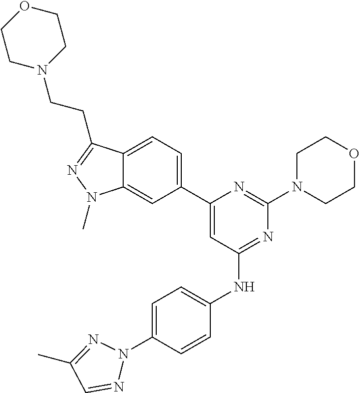 [SMILES: Cc1cnn(-c2ccc(Nc3cc(-c4ccc5c(CCN6CCOCC6)nn(C)c5c4)nc(N4CCOCC4)n3)cc2)n1]

### Step-1: Synthesis of (azidomethyl)benzene (2)

To a stirred solution of compound 1 (20 g, 1 eq) in acetone: water (4:1, 250 mL), sodium azide (11.4 g, 1.5 eq) was added and stirred for 12 h. The progress of the reaction was monitored by TLC. After completion of the reaction, the reaction mixture was quenched with water and extracted with DCM (3×50 mL). Combined organic extracts were washed with brine, dried over anhydrous sodium sulfate and evaporated under reduced pressure.

### Step-2: Synthesis of (1-benzyl-1H-1,2,3-triazol-4-yl)methanol (3)

To a stirred solution of propargyl alcohol (2.4 mL, 1.1 eq) and copper acetate (5 mL) in tert-butanol (30 mL), compound 2 (5 g, 1 eq) was added. The reaction mixture was degassed for 30 min and stirred for 12 h. The progress of the reaction was monitored by TLC. After completion of the reaction, the reaction mixture was evaporated under reduced pressure to obtain a crude residue. The crude product was purified by column chromatography on silica gel 100-200 mesh using 20% EtOAc-hexane to afford the compound.

### Step-3: Synthesis of 1-benzyl-4-(bromomethyl)-1H-1,2,3-triazole(4)

To a stirred solution of compound 3 (3 g, 1 eq) in DCM (120 mL), triphenyl phosphine (6.2 g, 1.5 eq) was added followed by addition of carbon tetra bromide (7.9 g, 1.5 eq) and stirred for 12 h. The progress of the reaction was monitored by TLC. After completion of the reaction, the reaction mixture was evaporated under reduced pressure to obtain a crude residue. The crude product was purified by column chromatography on silica gel 100-200 mesh using 25% EtOAc-hexane to afford the compound.

### Step-4: Synthesis of 4-methyl-1H-1,2,3-triazole(5,)

To a stirred solution of compound 4 (1.4 g, 1 eq) in ethanol: dioxane (1:1, 20 mL), 10% Pd—C (0.166 g) was added under nitrogen atmosphere. The reaction mixture was stirred at room temperature for 12 h under hydrogen balloon pressure. The progress of the reaction was monitored by TLC. After completion of the reaction, the reaction mixture was filtered through celite and evaporated under reduced pressure. The crude product was purified by column chromatography on silica gel 100-200 mesh using 40% ethyl acetate to afford the title compound. LCMS (m/z): 83.05(M+1).

### Step-5: Synthesis of 4-methyl-2-(4-nitrophenyl)-2H-1,2,3-triazole(7)

To a stirred solution of compound 6 (0.4 mL, 1 eq) in dry DMF (3 mL), NaH (0.211 g, 1.5 eq) was added at 0° C. and stirred for 15 min followed by the addition of compound 5 (0.380 g, 1.1 eq). The reaction mixture was stirred at room temperature for 30 min. The progress of the reaction was monitored by TLC. After completion of the reaction, the reaction mixture was quenched with water and extracted with ethyl acetate (3×50 mL). Combined organic extracts were washed with brine, dried over anhydrous sodium sulfate and evaporated under reduced pressure. The crude product was purified by column chromatography on silica gel 100-200 mesh using 0-5% EtOAc-hexane to afford the title compound. LCMS (m/z): 205 (M+1).

### Step-6: Synthesis of 4-(4-methyl-2H-1,2,3-triazol-2-yl)aniline(8)

To a stirred solution of compound 5 (0.4 g, 1 eq) in ethanol: THF (7 mL), 10% Pd—C (0.2 g) was added under nitrogen atmosphere. The reaction mixture was stirred at room temperature for 12 h under hydrogen balloon pressure. The progress of the reaction was monitored by TLC. After completion of the reaction, the reaction mixture was filtered through celite and evaporated under reduced pressure to afford the title compound. LCMS (m/z): 175(M+1).

### Step-7: Synthesis of N-(4-(4-methyl-2H-1,2,3-triazol-2-yl)phenyl)-6-(1-methyl-3-(2-morpholinoethyl)-1H-indazol-6-yl)-2-morpholinopyrimidin-4-amine

The title compound has been synthesized by following the general procedure described above for Buchwald coupling by using corresponding chloro compound 9 and amine 8. ¹H NMR (400 MHz, DMSO-d₆) δ 9.65 (s, 1H), 8.18 (s, 1H), 7.98-7.81 (m, 5H), 7.73 (d, J=9.5 Hz, 1H), 6.73 (s, 1H), 4.06 (s, 3H), 3.78 (dt, J=34.8, 4.8 Hz, 12H), 3.10 (s, 2H), 2.71 (s, 2H), 2.43 (s, 4H), 2.36 (s, 3H); HPLC purity: 98%; LCMS Calculated for C₃₄H₃₇N₇O₂: 575.70:observed: 581.55 (M+1).

### Example 388

### Synthesis of4-((6-(1,3-dimethyl-1H-indazol-6-yl)-2-morpholinopyrimidin-4-yl)amino)-N-methylbenzamide

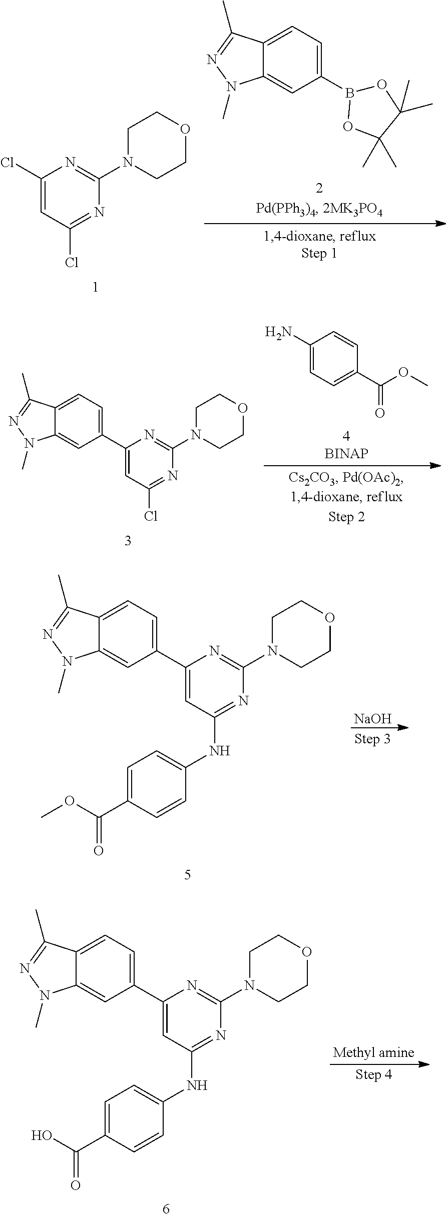

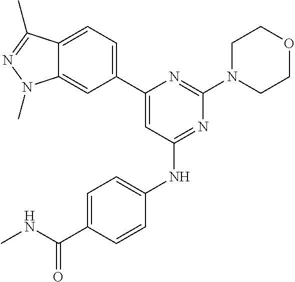 [SMILES: CNC(=O)c1ccc(Nc2cc(-c3ccc4c(C)nn(C)c4c3)nc(N3CCOCC3)n2)cc1]

Synthesis of4-((6-(1,3-dimethyl-1H-indazol-6-yl)-2-morpholinopyrimidin-4-yl)amino)-N-methylbenzamide: The title compound has been synthesized by following the general procedure described above for amide coupling by using corresponding acid compound 6 and amine. ¹H NMR (400 MHz, Methanol-d4) δ 8.02 (d, J=1.3 Hz, 1H), 7.97-7.86 (m, 3H), 7.77 (d, J=8.4 Hz, 2H), 7.49 (dd, J=8.4, 1.5 Hz, 1H), 6.62 (s, 1H), 4.10 (s, 3H), 3.95-3.83 (m, 8H), 2.94 (s, 3H), 2.60 (s, 3H); HPLC purity: 95.15%; LCMS Calculated for C₂₅H₂₇N₇O₂: 457.54:observed: 488.40 (M+1).

### Examples 389-425

The following compounds were prepared using methods disclosed herein or known to one of ordinary skill in the art.

N-(4-chlorophenyl)-6-(2-methyl-1-(2-(pyrrolidin-1-yl)ethyl)-1H-benzo[d]imidazol-5-yl)-2-morpholinopyrimidin-4-amine): ¹H NMR (400 MHz, DMSO-d₆) δ 9.47 (s, 1H), 8.14 (d, J=1.5 Hz, 1H), 7.86 (dd, J=8.4, 1.6 Hz, 1H), 7.75-7.66 (m, 2H), 7.56 (d, J=8.5 Hz, 1H), 7.40-7.31 (m, 2H), 6.61 (s, 1H), 4.30 (t, J=6.5 Hz, 2H), 3.81-3.67 (m, 8H), 3.37-3.32 (m, 2H), 2.76 (t, J=6.5 Hz, 3H), 1.72-1.60 (m, 4H); HPLC purity: 98.41%; LCMS Calculated for C₂₈H₃₂ClN₇O: 518.05:observed: 518.60(M+1).

Methyl 4-((6-(1-(2-methoxyethyl)-1H-benzo[d]imidazol-6-yl)-2-morpholinopyrimidin-4-yl) amino) benzoate: ¹H NMR (400 MHz, DMSO-d₆) δ: 9.83 (s, 1H), 8.30-8.22 (m, 2H), 7.97-7.82 (m, 5H), 7.73 (d, J=8.5 Hz, 1H), 6.74 (s, 1H), 4.50 (t, J=5.1 Hz, 2H), 3.86-3.69 (m, 13H), 3.25 (s, 3H); HPLC purity: 98.24%; LCMS Calculated for C₂₆H₂₈N₆O₄: 488.22; Observed: 489.30 (M+1).

4-((6-(1-(2-methoxyethyl)-1H-benzo[d]imidazol-6-yl)-2-morpholinopyrimidin-4-yl)amino)benzoic acid: ¹H NMR (400 MHz, DMSO-d₆) δ: 9.81 (s, 1H), 8.30-8.23 (m, 2H), 7.91 (d, J=8.6 Hz, 3H), 7.77 (dd, J=28.7, 8.5 Hz, 3H), 6.76 (s, 1H), 4.50 (t, J=5.3 Hz, 2H), 3.86-3.66 (m, 10H), 3.25 (s, 3H); HPLC purity: 99.79%; LCMS Calculated for C₂₅H₂₆N₆O₄: 474.20; Observed: 475.35 (M+1).

2-(2-(1-methyl-6-(6-((1-methyl-1H-indol-6-yl)amino)-2-morpholinopyrimidin-4-yl)-1H-indazol-3-yl)ethyl)isoindoline-1,3-dione: ¹H NMR (400 MHz, DMSO-d₆) δ 9.45 (s, 1H), 8.23(d, J=8.4 Hz, 2H), 7.92-7.65 (m, 6H), 7.45 (s, 1H), 7.21(s, 1H), 7.09 (d, J=8.5 Hz, 1H), 6.63 (s, 1H), 6.41(s, 1H),4.05-4.02 (m, 5H), 3.83 (d, J=4.9 Hz, 4H), 3.68-3.61 (m, 7H), 3.39-3.24 (m, 2H); HPLC purity: 98.59%; LCMS Calculated for C₃₅H₃₂N₈O₃: 612.69: Observed: 631 (M+1).

N-(4-chlorophenyl)-6-(3-ethyl-1-methyl-1H-pyrazolo[4,3-b]pyridin-6-yl)-2-morpholinopyrimidin-4-amine: ¹H NMR (400 MHz, DMSO-d₆) δ 9.65 (s, 1H), 9.08(s, 1H), 8.60(s,1H), 7.72 (d, J=8.4 Hz, 2H), 7.38 (d, J=8.5 Hz, 2H), 6.73 (s, 1H), 4.08 (s, 3H), 3.73 (d, J=4.9 Hz, 4H), 3.65 (s, 4H), 2.61 (s, 2H), 2.45 (s, 3H); HPLC purity: 92.32%; LCMS Calculated for C₂₃H₂₄ClN₇O: 449.94:observed : 450.30 (M+1).

N-(4-(2H-1,2,3-triazol-2-yl)phenyl)-6-(1,3-dimethyl-1H-pyrazolo[4,3-b]pyridin-6-yl)-2-morpholinopyrimidin-4-amine: ¹H NMR (400 MHz, DMSO-d₆) δ 10.09-10.02 (m, 1H), 9.03 (d, J=1.8 Hz, 1H), 8.64 (d, J=1.8 Hz, 1H), 8.09 (s, 2H), 8.06-7.97 (m, 2H), 7.94-7.86 (m, 2H), 6.80 (s, 1H), 4.10 (s, 3H), 3.85 (d, J=4.8 Hz, 4H), 3.75 (t, J=4.7 Hz, 4H), 2.57 (s, 3H); HPLC purity: 99.88%; LCMS Calculated for C₂₄H₂₄N₁₀O: 468.51:observed: 469.30(M+1).

N-(4-chlorophenyl)-6-(1H-indazol-4-yl)-2-morpholinopyrimidin-4-amine: ¹H NMR (400 MHz, DMSO-d₆) δ 8.47 (s, 1H), 7.75 (d, J=8.6 Hz, 3H), 7.63 (d, J=7.1 Hz, 1H), 7.55-7.38 (m, 3H), 6.81 (s, 1H), 3.84-3.71 (m, 8H), 2.46 (d, J=11.8 Hz, 1H); HPLC purity: 98.6%; LCMS Calculated for C₂₁H₁₉ClN₆O (free base): 406.87:observed : 407.20 (M+1).

Methyl 4-((6-(1-(2-methoxyethyl)-1H-indazol-6-yl)-2-morpholinopyrimidin-4-yl) amino) benzoate: ¹H NMR (400 MHz, DMSO-d₆) δ: 9.87 (s, 1H), 8.27 (s, 1H), 8.13 (s, 1H), 7.94 (d, J=8.5 Hz, 2H), 7.89-7.74 (m, 4H), 6.76 (s, 1H), 4.65 (t, J=5.3 Hz, 2H), 3.86-3.70 (m, 13H), 3.21 (s, 3H); HPLC purity: 99.13%; LCMS Calculated for C₂₆H₂₈N₆O₄: 488.22; Observed: 489.30 (M+1).

4-((6-(1-(2-methoxyethyl)-1H-indazol-6-yl)-2-morpholinopyrimidin-4-yl)amino)benzoic acid: ¹H NMR (400 MHz, DMSO-d₆-D₂O) δ: 8.18 (d, J=19.4 Hz, 2H), 7.91 (dd, J=21.1, 8.5 Hz, 3H), 7.80 (d, J=8.4 Hz, 2H), 7.65 (d, J=8.5 Hz, 1H), 4.62 (t, J=5.2 Hz, 2H), 3.79-3.69 (m, 10H), 3.17 (s, 3H); HPLC purity: 99.13%; LCMS Calculated for C₂₅H₂₆N₆O₄: 474.20; Observed: 475.25 (M+1).

2-(3-(1-methyl-6-(6-((1-methyl-1H-indol-6-yl)amino)-2-morpholinopyrimidin-4-yl)-1H-indazol-3-yl)propyl)isoindoline-1,3-dione: ¹H NMR (400 MHz, DMSO-d₆) δ 9.41 (s, 1H), 8.19 (s, 1H), 8.07 (s, 1H), 7.79 (d, J=12.7 Hz, 5H), 7.68 (dd, J=8.3, 1.3 Hz, 1H), 7.46 (d, J=8.3 Hz, 1H), 7.23 (d, J=3.1 Hz, 1H), 7.04 (dd, J=8.5, 1.8 Hz, 1H), 6.70 (s, 1H), 6.35 (d, J=3.0 Hz, 1H), 3.93 (s, 3H), 3.73 (q, J=7.3, 6.8 Hz, 9H), 3.33 (s, 4H), 2.98 (t, J=7.4 Hz, 2H), 2.49-2.42 (m, 1H), 2.13 (p, J=7.3 Hz, 2H); HPLC purity: 98.54%; LCMS Calculated for C₃₆H₃₄N₈O₃: 626.71:observed: 627.50(M+1).

N-(4-(5-cyclopropyl-4H-1,2,4-triazol-3-yl)phenyl)-6-(1-methyl-3-(2-morpholinoethyl)-1H-indazol-6-yl)-2-morpholinopyrimidin-4-amine: ¹H NMR (400 MHz, Chloroform-d) δ 8.04-7.97 (m, 3H), 7.75-7.63 (m, 2H), 7.53 (d, J=8.4 Hz, 2H), 6.68 (s, 1H), 6.56 (s, 1H), 4.07 (s, 3H), 3.92 (t, J=4.8 Hz, 4H), 3.81 (dt, J=11.6, 4.8 Hz, 8H), 3.28-3.18 (m, 2H), 2.92 (t, J=8.0 Hz, 2H), 2.68 (s, 4H), 2.04 (ddd, J=13.8, 8.3, 5.2 Hz, 1H), 1.19-1.04 (m, 4H); HPLC purity: 98.54%; LCMS Calculated for C₃₃H₃₈N₁₀O₂ (free base): 606.72:observed: 607.50(M+1).

6-(1,3-dimethyl-1H-indazol-6-yl)-N-(4-(1-(4-methoxybenzyl)-1H-pyrazol-4-yl)phenyl)-2-morpholinopyrimidin-4-amine: ¹H NMR (400 MHz, DMSO-d₆) δ 9.42 (s, 1H), 8.15 (s, 2H), 7.87-7.75 (m, 2H), 7.69 (dd, J=12.6, 8.4 Hz, 3H), 7.52 (d, J=8.2 Hz, 2H), 7.25 (d, J=8.3 Hz, 2H), 6.92 (d, J=8.3 Hz, 2H), 6.69 (s, 1H), 5.24 (s, 2H), 4.03 (s, 3H), 3.85-3.78 (m, 4H), 3.73 (d, J=6.9 Hz, 7H); HPLC purity: 96.20%; LCMS Calculated for C₃₄H₃₄N₈O₂ (free base): 586.69:observed: 587.50(M+1).

N-(4-chlorophenyl)-2-morpholino-6-(2-(2-morpholinoethyl)-1-(oxetan-3-yl)-1H-benzo[d] imidazol-6-yl)pyrimidin-4-amine: ¹H NMR (400 MHz, DMSO-d₆) δ 9.65 (s, 1H), 8.25 (s, 1H), 8.10 (s, 1H), 7.97-7.74 (m, 1H), 7.58 (d, J=7.7 Hz, 3H), 7.28(s, 1H),6.74 (d, J=1.9 Hz, 1H), 4.13 (d, J=2.0 Hz, 3H), 3.82 (d, J=5.0 Hz, 8H), 3.74 (t, J=4.7 Hz, 4H), 3.05 (d, J=2 Hz, 2H), 2.86 (d, J=1.9 Hz, 2H), 2.36 (d, J=1.9 Hz, 4H); HPLC purity: 98.23%; LCMS Calculated for C₃₀H₃₄ClN₇O₃: 576.10; Observed: 576.45 (M+1).

N-(6-(1-(cyclopropylmethyl)-2-(2-morpholinoethyl)-1H-benzo[d]imidazol-6-yl)-2-morpholinopyrimidin-4-yl)-1-methyl-1H-indol-6-amine: ¹H NMR (400 MHz, DMSO-d₆) δ 9.33 (s, 1H), 8.17 (s, 1H), 8.12 (s, 1H), 7.83 (d, J=8.6 Hz, 1H), 7.60 (d, J=8.4 Hz, 1H), 7.44 (d, J=8.5 Hz, 1H), 7.21 (d, J=3.0 Hz, 1H), 7.03 (d, J=8.5 Hz, 1H), 6.65 (s, 1H), 6.33 (d, J=3.0 Hz, 1H), 4.20 (d, J=6.8 Hz, 2H), 3.84 (t, J=4.5 Hz, 4H), 3.58 (t, J=4.6 Hz, 7H), 3.30 (d, J=1.9 Hz, 4H), 3.07 (t, J=7.6 Hz, 2H), 2.84 (t, J=7.6 Hz, 2H), 2.36-2.31(m, 4H)1.27-1.19 (m, 1H), 0.54 (d, J=7.8 Hz, 2H), 0.46 (d, J=5.0 Hz, 2H); HPLC purity: 98.91%; LCMS Calculated for C₃₄H₄₀N₈O₂: 592.75; Observed: 593.55 (M+1).

6-(1-methyl-1H-indazol-6-yl)-N-(4-(4-methyl-2H-1,2,3-triazol-2-yl)phenyl)-2-morpholino pyrimidin-4-amine: ¹H NMR (400 MHz, DMSO-d₆) δ 9.65 (s, 1H), 8.25 (s, 1H), 8.10 (s, 1H), 7.97-7.74 (m, 7H), 6.74 (d, J=1.9 Hz, 1H), 4.13 (d, J=2.0 Hz, 3H), 3.82 (d, J=5.0 Hz, 4H), 3.74 (t, J=4.7 Hz, 4H), 2.36 (d, J=1.9 Hz, 3H); HPLC purity: 93.15%; LCMS Calculated for C₂₅H₂₅N₉O: 467.54; Observed:468.35 (M+1).

6-(1,3-dimethyl-1H-indazol-6-yl)-N-(4-(4-methyl-2H-1,2,3-triazol-2-yl)phenyl)-2-morpholinopyrimidin-4-amine(: ¹H NMR (400 MHz, DMSO-d₆) δ 9.63 (s, 1H), 8.16 (s, 1H), 7.96-7.69 (m, 7H), 6.72 (s, 1H), 4.04 (s, 3H), 3.82 (d, J=4.6 Hz, 4H), 3.77-3.70 (m, 4H), 2.46 (s, 3H),2.35 (s, 3H); HPLC purity: 99.02%; LCMS Calculated for C₂₆H₂₇N₉O: 481.56; Observed:482.40 (M+1).

1,3-dimethyl-N-(6-(1-methyl-3-(2-morpholinoethyl)-1H-indazol-6-yl)-2-morpholinopyrimidin-4-yl)-1H-indol-6-amine: ¹H NMR (400 MHz, DMSO-d₆) δ 9.38 (s, 1H), 8.15 (s, 1H), 8.09 (s, 1H), 7.84 (d, J=8.5 Hz, 1H), 7.74-7.66 (m, 1H), 7.41 (d, J=8.4 Hz, 1H), 7.09-6.96 (m, 2H), 6.71 (s, 1H), 4.04 (s, 3H), 3.90-3.82 (m, 4H), 3.78-3.55 (m, 11H), 3.09 (dd, J=8.9, 6.8 Hz, 2H), 2.70 (dd, J=8.9, 6.8 Hz, 2H), 2.22 (s, 3H); HPLC purity: 96.76%; LCMS Calculated for C₃₂H₃₈N₈O₂: 566.71; Observed: 567.45 (M+1).

1,2-dimethyl-N-(6-(1-methyl-3-(2-morpholinoethyl)-1H-indazol-6-yl)-2-morpholino pyrimidin-4-yl)-1H-indol-6-amine: ¹H NMR (400 MHz, DMSO-d₆) δ 9.32 (s, 1H), 8.14 (s, 1H), 8.06 (s, 1H), 7.83 (d, J=8.4 Hz, 1H), 7.73-7.66 (m, 1H), 7.34 (d, J=8.3 Hz, 1H), 6.99 (dd, J=8.4, 1.8 Hz, 1H), 6.69 (s, 1H), 6.14 (s, 1H), 4.04 (s, 3H), 3.90-3.78 (m, 4H), 3.74 (t, J=4.9 Hz, 4H), 3.63-3.55 (m, 7H), 3.13-3.04 (m, 2H), 2.70 (dd, J=8.9, 6.9 Hz, 2H), 2.47 (d, J=4.6 Hz, 4H), 2.39 (s, 3H); HPLC purity: 95.74%; LCMS Calculated for C₃₂H₃₈N₈O₂: 566.71; Observed: 567.40 (M+1).

N-(6-(1,3-dimethyl-1H-indazol-6-yl)-2-morpholinopyrimidin-4-yl)-1,2-dimethyl-1H-indol-6-amine: ¹H NMR (400 MHz, DMSO-d₆) δ 9.32 (s, 1H), 8.14 (s, 1H), 8.06 (s, 1H), 7.78 (d, J=8.5 Hz, 1H), 7.73-7.66 (m, 1H), 7.34 (d, J=8.4 Hz, 1H), 6.99 (dd, J=8.4, 1.9 Hz, 1H), 6.69 (s, 1H), 6.14 (s, 1H), 4.03 (d, J=1.5 Hz, 3H), 3.86 (t, J=4.7 Hz, 4H), 3.74 (t, J=4.5 Hz, 4H), 3.61 (d, J=1.5 Hz, 3H), 2.39 (s, 3H), 2.21 (m, 3H); HPLC purity: 96.83%; LCMS Calculated for C₂₇H₂₉N₇O: 467.58; Observed: 468.35 (M+1).

4-chloro-N-(6-(1,3-dimethyl-1H-indazol-6-yl)-2-morpholinopyrimidin-4-yl)-1-methyl-1H-indol-6-amine: ¹H NMR (400 MHz, DMSO-d₆) δ 9.62 (s, 1H), 8.25 (d, J=2.5 Hz, 2H), 8.16 (d, J=2.4 Hz, 2H), 7.83-7.68 (m, 2H), 7.62 (dd, J=9.1, 2.4 Hz, 1H), 6.73 (d, J=2.4 Hz, 1H), 4.03 (d, J=2.5 Hz, 3H), 3.85-3.78 (m, 4H), 3.74 (d, J=4.9 Hz, 7H), 2.50 (dd, J=3.6, 1.9 Hz, 3H); HPLC purity: 97.19%; LCMS Calculated for C₂₆H₂₆ClN₇O: 487.99; Observed: 488.30 (M+1).

N-(6-(3-(2-aminoethyl)-1-methyl-1H-indazol-6-yl)-2-morpholinopyrimidin-4-yl)-1-methyl-1H-indol-6-amine: ¹H NMR (400 MHz, Methanol-d₄) δ 8.18 (t, J=1.1 Hz, 1H), 7.98 (s, 1H), 7.79 (d, J=1.0 Hz, 1H), 7.48 (d, J=8.4 Hz, 1H), 7.13-7.04 (m, 2H), 6.64 (s, 1H), 6.39 (dd, J=3.2, 0.9 Hz, 1H), 4.11 (s, 3H), 3.94 (t, J=4.8 Hz, 4H), 3.85-3.75 (m, 8H), 3.67-3.58 (m, 3H), 3.43 (dd, J=7.2, 5.6 Hz, 2H), 3.38-3.31 (m, 2H); HPLC purity: 96.64%; LCMS Calculated for C₂₇H₃₀N₈O: 482.59; Observed: 483.30 (M+1).

2-(2-(1-methyl-6-(6-((1-methyl-1H-indol-6-yl)amino)-2-morpholinopyrimidin-4-yl)-1H-indazol-3-yl)ethyl)isoindoline-1,3-dione: ¹H NMR (400 MHz, DMSO-d₆) δ 9.56 (s, 1H), 8.16(d, J=2.4 Hz, 2H), 7.88-7.66 (m, 6H), 7.25(s, 1H), 7.21(s,1H), 7.08(d, J=8.4 Hz, 1H),6.72 (s, 1H), 6.51 (d, J=2.3 Hz, 1H), 4.02-3.90 (m, 4H), 3.78 (dq, J=28.9, 4.6 Hz, 4H), 3.68 (d, J=3.5 Hz, 7H),3.28 (s, 2H); HPLC purity: 98.59%; LCMS Calculated for C₃₅H₃₂N₈O₃: 612.69; Observed: 613 (M+1).

N-(6-(3-(3-(dimethylamino)propyl)-1-methyl-1H-indazol-6-yl)-2-morpholinopyrimidin-4-yl)-1-methyl-1H-indol-6-amine: ¹H NMR (400 MHz, Methanol-d₄) δ 8.13 (s, 1H), 7.99 (s, 1H), 7.76 (q, J=8.3 Hz, 2H), 7.47 (d, J=8.4 Hz, 1H), 7.12-7.03 (m, 2H), 6.63 (d, J=2.5 Hz, 1H), 6.38 (d, J=3.2 Hz, 1H), 4.06 (d, J=3.3 Hz, 3H), 3.85-3.74 (m, 1H), 3.02 (t, J=7.4 Hz, 2H), 2.62-2.53 (m, 2H), 2.37 (s, 6H), 2.04 (p, J=7.8 Hz, 2H); HPLC purity: 96.38%; LCMS Calculated for C₃₀H₃₆N₈O: 524.67; Observed: 525.40 (M+1).

1-methyl-N-(6-(1-methyl-3-(3-(pyrrolidin-1-yl)propyl)-1H-indazol-6-yl)-2-morpholino pyrimidin-4-yl)-1H-indol-6-amine: ¹H NMR (400 MHz, DMSO-d₆) δ 9.43 (s, 1H), 8.29 (d, J=1.3 Hz, 1H), 8.27-8.16 (m, 2H), 7.87 (dd, J=8.6, 1.5 Hz, 1H), 7.47 (d, J=8.4 Hz, 1H), 7.24 (d, J=3.1 Hz, 1H), 7.05 (dd, J=8.5, 1.9 Hz, 1H), 6.75 (s, 1H), 6.35 (d, J=3.1 Hz, 1H), 4.12 (s, 3H), 3.92-3.84 (m, 4H), 3.75 (d, J=3.5 Hz, 7H), 2.98-2.95 (m, 2H), 2.46-2.41 (m, 6H), 1.91 (dp, J=32.2, 7.1 Hz, 2H), 1.65-1.6(m, 4H); HPLC purity: 94.8%; LCMS Calculated for C₃₂H₃₈N₈O: 550.71; Observed: 551.45 (M+1).

4-(4-((6-(1-methyl-1H-indazol-6-yl)-2-morpholinopyrimidin-4-yl)amino)phenyl)-2,4-dihydro-3H-1,2,4-triazol-3-one: ¹H NMR (400 MHz, DMSO-d₆) δ 11.93 (s, 1H), 9.61 (s, 1H), 8.32 (d, J=1.4 Hz, 1H), 8.24 (s, 1H), 8.10 (d, J=1.1 Hz, 1H), 7.89-7.73 (m, 4H), 7.61 (d, J=8.7 Hz, 2H), 6.73 (s, 1H), 4.13 (d, J=1.2 Hz, 3H), 3.78 (dt, J=35.4, 4.5 Hz, 8H); HPLC purity: 97.84%; LCMS Calculated for C₂₄H₂₃N₉O₂: 469.51; Observed: 470.35 (M+1).

N-(4-(1-methyl-1H-1,2,3-triazol-4-yl)phenyl)-6-(1-methyl-1H-indazol-6-₃71)-2-morpholino pyrimidin-4-amine: ¹H NMR (400 MHz, DMSO-d₆) δ 9.55 (s, 1H), 8.42 (s, 1H), 8.24 (s, 1H), 8.10 (s, 1H), 7.90-7.74 (m, 6H), 6.74 (s, 1H), 4.11 (d, J=19.2 Hz, 6H), 3.87-3.74 (m, 8H), 2.46 (d, J=8.3 Hz, 1H); HPLC purity: 99.63%; LCMS Calculated for C₂₅H₂₅N₉O: 467.54; Observed: 468 (M+1).

1,2,3-trimethyl-N-(6-(1-methyl-1H-indazol-6-yl)-2-morpholinopyrimidin-4-yl)-1H-indol-6-amine: ¹H NMR (400 MHz, DMSO-d₆) δ 8.79 (s, 1H), 8.31(s, 1H),8.03 (s, 1H), 7.69 (d, J=8.4 Hz, 1H), 7.49 (d, J=8.4 Hz, 1H), 7.24 (d, J=8.1 Hz, 1H), 7.10-6.94 (m, 2H), 6.35 (s, 1H), 3.98 (s, 3H), 3.76-3.62 (m, 8H),3.19 (s, 3H), 2.26 (s, 3H), 1.96 (s, 3H); HPLC purity: 96.62%; LCMS Calculated for C₂₇H₂₉N₇O: 467.58; Observed: 468.35 (M+1).

N-(4-(1H-pyrazol-1-yl)phenyl)-6-(1-methyl-1H-indazol-6-yl)-2-morpholinopyrimidin-4-amine: ¹H NMR (400 MHz, DMSO-d₆) δ 9.57 (s, 1H), 8.42 (d, J=2.5 Hz, 1H), 8.24 (s, 1H), 8.10 (s, 1H), 7.89-7.74 (m, 6H), 7.71 (d, J=1.8 Hz, 1H), 6.73 (s, 1H), 4.13 (s, 3H), 3.78 (dt, J=36.9, 4.6 Hz, 8H), 2H); HPLC purity: 95.07%; LCMS Calculated for C₂₅H₂₄N₈O: 452.52; Observed: 453.30 (M+1).

6-(1-methyl-1H-indazol-6-yl)-N-(4-(4-methyl-1H-pyrazol-1-yl)phenyl)-2-morpholino pyrimidin-4-amine¹H NMR (400 MHz, DMSO-d₆) δ 9.54 (s, 1H), 8.26-8.16 (m, 2H), 8.09 (d, J=1.0 Hz, 1H), 7.89-7.69 (m, 6H), 7.52 (s, 1H), 6.72 (s, 1H), 4.13 (s, 3H), 3.78 (dt, J=36.0, 4.7 Hz, 8H), 2.10 (s, 3H); HPLC purity: 97.08%; LCMS Calculated for C₂₆H₂₆N₈O: 466.55; Observed: 467.30 (M+1).

6-(1-methyl-1H-indazol-6-yl)-N-(4-(3-methyl-1H-pyrazol-1-yl)phenyl)-2-morpholinopyrimidin-4-amine: ¹H NMR (400 MHz, DMSO-d₆) δ 9.55 (s, 1H), 8.32-8.21 (m, 2H), 8.10 (s, 1H), 7.91-7.70 (m, 6H), 7.67-7.51 (m, 1H), 6.71 (s, 1H), 6.30 (d, J=2.3 Hz, 1H), 4.13 (s, 3H), 3.78 (dt, J=35.5, 4.6 Hz, 8H), 2.27 (s, 3H); HPLC purity: 96.09%; LCMS Calculated for C₂₆H₂₆N₈O: 466.55; Observed: 467.25 (M+1).

N-(4-(1H-pyrazol-1-yl)phenyl)-6-(1,3-dimethyl-1H-indazol-6-yl)-2-morpholinopyrimidin-4-amine: ¹H NMR (400 MHz, DMSO-d₆) δ 9.57 (s, 1H), 8.42 (d, J=2.5 Hz, 1H), 8.24 (s, 1H), 7.89-7.74 (m, 7H), 6.73 (s, 1H), 6.52 (t, J=2.1 Hz, 1H), 4.13 (s, 3H), 3.78 (dt, J=36.9, 4.6 Hz, 8H), 2.46 (s, 3H); HPLC purity: 94.51%; LCMS Calculated for C₂₆H₂₆N₈O: 466.55; Observed: 467.35 (M+1).

6-(1,3-dimethyl-1H-indazol-6-yl)-N-(4-(4-methyl-1H-pyrazol-1-yl)phenyl)-2-morpholino pyrimidin-4-amine: ¹H NMR (400 MHz, DMSO-d₆) δ 9.54 (s, 1H), 8.09 (d, J=1.0 Hz, 2H), 7.89-7.69 (m, 6H), 7.52 (s, 1H), 6.72 (s, 1H), 4.13 (s, 3H), 3.78 (dt, J=36.0, 4.7 Hz, 8H), 2.46(s, 3H),2.10 (s, 3H); HPLC purity: 97.06%; LCMS Calculated for C₂₇H₂₈N₈O: 480.58; Observed: 481.35 (M+1).

6-(1,3-dimethyl-1H-indazol-6-yl)-N-(4-(3-methyl-1H-pyrazol-1-yl)phenyl)-2-morpholino pyrimidin-4-amine: ¹H NMR (400 MHz, DMSO-d₆) δ 9.43 (s, 1H), 8.29 (d, J=1.3 Hz, 1H), 8.16 (s, 1H), 7.87-7.67 (d, J=8.4 Hz, 6H), 6.75 (s, 1H), 6.35 (d, J=3.1 Hz, 1H), 4.08 (s, 3H), 3.92-3.84 (m, 8H), 2.46 (s, 3H), 2.12 (s, 3H); HPLC purity: 95%; LCMS Calculated for C₂₇H₂₈N₈O: 480.58; Observed: 481.30 (M+1).

N-(4-(1H-pyrazol-1-yl)phenyl)-6-(1-methyl-3-(2-morpholinoethyl)-1H-indazol-6-yl)-2-morpholinopyrimidin-4-amine: ¹H NMR (400 MHz, DMSO-d₆) δ 9.56 (s, 1H), 8.42 (d, J=2.4 Hz, 1H), 8.16 (s, 1H), 7.88-7.66 (m, 7H), 6.72 (s, 1H), 6.51 (d, J=2.3 Hz, 1H), 4.05 (s, 3H), 3.78 (dq, J=28.9, 4.6 Hz, 8H), 3.58 (q, J=7.6, 6.0 Hz, 4H), 3.28 (s, 2H), 3.09 (dd, J=9.4, 6.3 Hz, 2H), 2.56 (d, J=18.9 Hz, 2H), 2.47 (d, J=5.5 Hz, 4H); HPLC purity: 97.54%; LCMS Calculated for C₃₁H₃₅N₉O₂: 565.68; Observed: 566.45 (M+1).

N-(4-(3-methyl-1H-pyrazol-1-yl)phenyl)-6-(1-methyl-3-(2-morpholinoethyl)-1H-indazol-6-yl)-2-morpholinopyrimidin-4-amine: ¹H NMR (400 MHz, DMSO-d₆) δ 9.52 (s, 1H), 8.28 (d, J=2.4 Hz, 1H), 8.16 (s, 1H), 7.92-7.68 (m, 6H), 6.70 (s, 1H), 6.30 (d, J=2.3 Hz, 1H), 4.05 (s, 3H), 3.81 (q, J=6.0, 5.2 Hz, 4H), 3.73 (t, J=4.7 Hz, 4H), 3.63-3.56 (m, 4H), 3.14-3.04 (m, 2H), 2.72 (d, J=8.6 Hz, 2H), 2.50 (s, 4H), 2.27 (s, 3H); HPLC purity: 93.48%; LCMS Calculated for C₃₂H₃₇N₉O₂: 579.71; Observed: 580.45 (M+1).

N-(4-chlorophenyl)-6-(2-(2-(dimethylamino)ethyl)-3-methyl-2H-indazol-6-yl)-2-morpholinopyrimidin-4-amine: ¹H NMR (400 MHz, MeOD) δ: 8.21 (s, 1H), 7.74-7.63 (m, 4H), 7.33-7.24 (m, 2H), 6.58 (s, 1H), 4.53 (t, J=6.9 Hz, 2H), 3.83-3.74 (m, 8H), 2.93 (t, J=6.9 Hz, 2H), 2.70 (s, 3H), 2.34 (s, 6H); HPLC purity: 97.92%; LCMS Calculated for C₂₆H₃₀ClN₇O: 491.22; Observed: 492.40 (M+1).

Methyl 4-((6-(1-methyl-1H-indazol-6-yl)-2-morpholinopyrimidin-4-yl) amino) benzoate: ¹H NMR (400 MHz, DMSO-d₆) δ: 9.87 (s, 1H), 8.25 (s, 1H), 8.10 (s, 1H), 7.94 (d, J=8.4 Hz, 2H), 7.86 (d, J=8.2 Hz, 3H), 7.78 (d, J=8.5 Hz, 1H), 6.79 (s, 1H), 4.13 (s, 3H), 3.82-3.74 (m, 11H); HPLC purity: 94.79%; LCMS Calculated for C₂₄H₂₄N₆O₃: 444.19; Observed: 445.25 (M+1).

### BIOLOGICAL EXAMPLES

The following describes ways in which the compounds described herein were tested to measure in vitro activity in cell-based assays. A person of ordinary skill in the art would know that variations in the assay conditions could be used to determine the activity of the compounds. See Nat. Chem Biol. 2012, 8, 197.

### Assay 1: MPS Cellular Assay

Dermal fibroblasts from humans with an MPS disorder and from healthy donors are grown. Cells are expanded and kept in culture for analysis. Human foreskin fibroblasts can be obtained from the American Type Culture Collection (CRL-1634). Human dermal fibroblasts derived from biopsies of individuals are purchased from Coriell Institute: clinically healthy individuals, GM00408, GM00409, GM00200 (clinically unaffected sibling of an individual with metachromatic leukodystrophy), GM05659, GM08398, and GM15871 (clinically unaffected sibling of an individual with unclassified Ehlers-Danlos syndrome); heterozygous MPS I carriers, GM01392, GM01393 and GM0003; homozygous individuals affected with MPS I, GM06214, GM11495, GM00034, GM01391 and GM01256; individuals with MPS II, GM01896, GM03181, GM00615 and GM00298; individuals with MPS IIIA, GM00879, GM00643, GM00934, GM06110 and GM00629; MPS IIIB, GM01426; MPS IIIC, GM05157; MPS IIID, GM05093; and MPS VII, GM00121. All cells are grown in DMEM containing 50 U ml-1 penicillin, 50 pg ml-1 streptomycin, 2 mM glutamine and 10% FBS. Cells are seeded on 15-cm-diameter tissue culture dishes, grown to confluence and maintained in culture for analysis. Sulfamidase activity in cell extracts is measured with 4-methylumbelliferyl-α-D-N-sulfoglucosaminide according the vendors instructions substituting Tris-acetate buffer (pH 6.5; Moscerdam).

Cells are treated with a compound of the invention at a concentration at or below 30 μM for 4 to 21 days. Cell monolayers are washed with phosphate buffered saline and detached by treatment with GIBCO trypsin-EDTA solution (Invitrogen). After centrifugation, the supernatant is removed and the cells are lysed in 0.5 mL of 0.1 N sodium hydroxide. The amount of protein is determined by bicinchoninic acid (BCA assay, Thermo Scientific). GAGs are isolated by anion exchange chromatography and digested with glycosaminoglycan lyases (heparinases I, II, and III, IBEX).

Enzymatically depolymerized GAG preparations are differentially mass labeled by reductive amination with [¹²C₆]aniline. Briefly, heparan sulfate disaccharides (1-10 pmol) are dried down in a centrifugal evaporator and reacted with [¹²C₆]aniline or [¹³C₆]aniline (15 μL, 165 μmol) and 15 μL of 1 M NaCNBH₃ (Sigma-Aldrich) freshly prepared in dimethylsulfoxide/acetic acid (7:3, v/v) is added to each sample. Reactions are carried out at 37° C. for 16 h and then dried in a centrifugal evaporator. Unsubstituted amines are reacted with propionic anhydride (Sigma-Aldrich). Dried samples are reconstituted in 20 μL of 50% methanol, and 3 μL of propionic anhydride (Sigma-Aldrich, 23.3 μmol) is added. Reactions are carried out at ˜23° C. for 2 h. Acylated disaccharides are subsequently aniline tagged as described above. Each sample is mixed with commercially available standard unsaturated disaccharides (Seikagaku), standard N-sulfoglucosamine, glucosamine-6-sulfate, N-acetylgalactosamine-4-sulfate and N-acetylgalactosamine-6-sulfate (Sigma-Aldrich), and/or synthesized α-L-idopyranosyluronate-(1,4)-2-N-acetyl-2-deoxy-α/β-D-glucopyranoside (I0S0). All standards are tagged with [¹³C₆]aniline (Sigma-Aldrich). Samples are then analyzed by LC-MS using an LTQ Orbitrap Discovery electrospray ionization mass spectrometer (Thermo Scientific) equipped with quaternary HPLC pump (Finnigan Surveyor MS pump) and a reverse-phase capillary column as described in J. Biol. Chem. 2008, 283(48), 33674.

MPS II: Assay 1, as described above, was used to determine activity for MPS II using GM01896 cells. The cells were treated with a compound of the invention and the heparan sulfate disaccharide detected was [¹²C₆]aniline-tagged I2a4, I2a6, 12S0 and/or I2S6. Extracted ion chromatograms were obtained. Cell viability was monitored using alamar blue or ATP quantification. IC50's were calculated using curve fitting within the ChemInnovation CBIS database system. Data is given in Table 2.

Assay 1 can be adapted for use in testing cellular activity of compounds of the invention for treating MPS I where the biomarker detected is I0a6, I0a4, I0a10, I0S0 and/or I0S6; MPS IIIA where the biomarker detected is S0, S0U0S6, S0U2A6, S0U2S0, S0U2S6, S6U0A6, S6U0S0, S6U0S6, S6U2A6, S6U2S0, and/or S6U2S6; MPSIIIB where the biomarker detected is A0U0A6, A0U0S6, A0U2A0, A0U2S0, and/or A0U2S6; MPS IIIC where the biomarker detected is H0U0S6, H0U2A6, H0U2S0, H0U2S6, H6U0A6, H6U0S0, H6U0S6, H6U2A6, H6U2S0, and/or H6U2S6; MPS IIID where the biomarker detected is H6; and MPS VII where the biomarker detected is G0a0, G0a4, G0a10, G0a6, G0A0, G0A6, G0S0, and/or G0S6. Saccharide structure naming code is as described in Lawrence et al. Nature Methods 2008, 5(4), 291. Certain embodiments have been evaluated in Assay I in MPSIIIA cells and demonstrated activity in the range of about 0.2 μM to about 2 μM.

The compounds of Formula I have the ability to reduce heparan sulfate accumulation as shown by Assay 1 in which the compounds reduced biomarker(s) indicative of abnormal HS accumulation in MPS II cells. Certain embodiments have also been evaluated in MPS IIIA cells and demonstrated a reduction in biomarker(s) indicative of abnormal HS accumulation. The compounds in the following table show activity in Assay 1 for MPS II, where activity “A” represents an IC₅₀ of less than 1 to 500 nM; “B” represents an IC₅₀ greater than 500 to 1000 nM, and “C” represents an IC₅₀ greater than 1000 to 5000 nM. A person of ordinary skill in the art would recognize that the compounds have the ability to modulate HS biosynthesis in general which enables the degradation of the heparan sulfate polymer and therefore are useful for treating diseases associated with abnormal HS accumulation.

TABLE 2

| | IC₅₀ |
|---|---|
| Compound | Range |
| (2-methoxy(4-pyridyl))[6-(1-methylbenzimidazol-6-yl)-2-morpholin-4- | B |
| ylpyrimidin-4-yl]amine | |
| N-methyl(5-{[6-(1-methyl(1H-indazol-6-yl))-2-morpholin-4-ylpyrimidin-4- | B |
| yl]amino}(2-pyridyl))carboxamide | |
| N-methyl(5-{[6-(1-methylbenzimidazol-5-yl)-2-morpholin-4-ylpyrimidin-4- | B |
| yl]amino}(2-pyridyl))carboxamide | |
| N-methyl(5-{[6-(1-methylbenzimidazol-6-yl)-2-morpholin-4-ylpyrimidin-4- | B |
| yl]amino}(2-pyridyl))carboxamide | |
| N-methyl(5-{[6-(1-methyl(1H-indazol-5-yl))-2-morpholin-4-ylpyrimidin-4- | B |
| yl]amino}(2-pyridyl))carboxamide | |
| (4-chlorophenyl)[6-(1-methylbenzimidazol-6-yl)-2-morpholin-4-ylpyrimidin- | A |
| 4-yl]amine | |
| (4-chlorophenyl)[6-(1-methylbenzimidazol-5-yl)-2-morpholin-4-ylpyrimidin- | B |
| 4-yl]amine | |
| (4-chlorophenyl)[6-(1-methyl(1H-indazol-6-yl))-2-morpholin-4-ylpyrimidin- | B |
| 4-yl]amine | |
| (2-methoxy(4-pyridyl))[6-(1-methyl(1H-indazol-5-yl))-2-morpholin-4- | C |
| ylpyrimidin-4-yl]amine | |
| [6-(1-methyl(1H-indazol-6-yl))-2-morpholin-4-ylpyrimidin-4-yl](4- | C |
| methylphenyl)amine | |
| (2-methoxy(4-pyridyl))[6-(1-methylbenzimidazol-5-yl)-2-morpholin-4- | C |
| ylpyrimidin-4-yl]amine | |
| [6-(1-methylbenzimidazol-6-yl)-2-morpholin-4-ylpyrimidin-4-yl](4- | C |
| methylphenyl)amine | |
| [6-(1-methyl(1H-indazol-5-yl))-2-morpholin-4-ylpyrimidin-4-yl](4- | C |
| methylphenyl)amine | |
| (2-methoxy(4-pyridyl))[6-(1-methyl(1H-indazol-6-yl))-2-morpholin-4- | B |
| ylpyrimidin-4-yl]amine | |
| [6-(1-methylbenzimidazol-5-yl)-2-morpholin-4-ylpyrimidin-4-yl](4- | B |
| methylphenyl)amine | |
| (6-(1H-indazol-6-yl)-2-morpholin-4-ylpyrimidin-4-yl)(4-chlorophenyl)amine | C |
| (4-chlorophenyl){6-[1-(cyclopropylmethyl)(1H-indazol-6-yl)]-2-morpholin-4- | C |
| ylpyrimidin-4-yl}amine | |
| (4-chlorophenyl)[6-(1-cyclopropyl(1H-indazol-6-yl))-2-morpholin-4- | C |
| ylpyrimidin-4-yl]amine | |
| (4-chlorophenyl){6-[2-(cyclopropylmethyl)(2H-indazol-6-yl)]-2-morpholin-4- | C |
| ylpyrimidin-4-yl}amine | |
| (5-{[6-(1-cyclopropyl(1H-indazol-6-yl))-2-morpholin-4-ylpyrimidin-4- | A |
| yl]amino}(2-pyridyl))-N-methylcarboxamide | |
| (4-chlorophenyl){6-[2-(cyclopropylmethyl)(2H-indazol-5-yl)]-2-morpholin-4- | C |
| ylpyrimidin-4-yl}amine | |
| (6-(1H-indazol-5-yl)-2-morpholin-4-ylpyrimidin-4-yl)(4-chlorophenyl)amine | C |
| [5-({6-[2-(cyclopropylmethyl)(2H-indazol-6-yl)]-2-morpholin-4-ylpyrimidin- | C |
| 4-yl}amino)(2-pyridyl)]-N-methylcarboxamide | |
| N-methyl(4-{[6-(1-methyl(1H-indazol-5-yl))-2-morpholin-4-ylpyrimidin-4- | B |
| yl]amino}phenyl)carboxamide | |
| {5-[(6-(1H-indazol-5-yl)-2-morpholin-4-ylpyrimidin-4-yl)amino](2-pyridyl)}- | B |
| N-methylcarboxamide | |
| [5-({6-[2-(cyclopropylmethyl)(2H-indazol-5-yl)]-2-morpholin-4-ylpyrimidin- | C |
| 4-yl}amino)(2-pyridyl)]-N-methylcarboxamide | |
| N-methyl(4-{[6-(1-methyl(1H-indazol-6-yl))-2-morpholin-4-ylpyrimidin-4- | A |
| yl]amino}phenyl)carboxamide | |
| (6-benzimidazol-6-yl-2-morpholin-4-ylpyrimidin-4-yl)(4-chlorophenyl)amine | B |
| N-methyl(4-{[6-(1-methylbenzimidazol-6-yl)-2-morpholin-4-ylpyrimidin-4- | A |
| yl]amino}phenyl)carboxamide | |
| N-methyl(4-{[6-(1-methylbenzimidazol-5-yl)-2-morpholin-4-ylpyrimidin-4- | A |
| yl]amino}phenyl)carboxamide | |
| (4-chlorophenyl){6-[1-(cyclopropylmethyl)benzimidazol-5-yl]-2-morpholin-4- | B |
| ylpyrimidin-4-yl}amine | |
| N-cyclopropyl(5-{[6-(1-methylbenzimidazol-6-yl)-2-morpholin-4- | B |
| ylpyrimidin-4-yl]amino}(2-pyridyl))carboxamide | |
| {5-[(6-(1H-indazol-6-yl)-2-morpholin-4-ylpyrimidin-4-yl)amino](2-pyridyl)}- | B |
| N-methylcarboxamide | |
| {5-[(6-benzimidazol-6-yl-2-morpholin-4-ylpyrimidin-4-yl)amino](2- | C |
| pyridyl)}-N-methylcarboxamide | |
| (4-chlorophenyl)[6-(1-cyclopropylbenzimidazol-6-yl)-2-morpholin-4- | B |
| ylpyrimidin-4-yl]amine | |
| (4-chlorophenyl){6-[1-(cyclopropylmethyl)benzimidazol-6-yl]-2-morpholin-4- | B |
| ylpyrimidin-4-yl}amine | |
| [5-({6-[1-(cyclopropylmethyl)benzimidazol-5-yl]-2-morpholin-4-ylpyrimidin- | B |
| 4-yl}amino)(2-pyridyl)]-N-methylcarboxamide | |
| (4-chlorophenyl)[6-(1-cyclopropylbenzimidazol-5-yl)-2-morpholin-4- | C |
| ylpyrimidin-4-yl]amine | |
| (5-{[6-(1-cyclopropylbenzimidazol-6-yl)-2-morpholin-4-ylpyrimidin-4- | B |
| yl]amino}(2-pyridyl))-N-methylcarboxamide | |
| [5-({6-[1-(cyclopropylmethyl)benzimidazol-6-yl]-2-morpholin-4-ylpyrimidin- | A |
| 4-yl}amino)(2-pyridyl)]-N-methylcarboxamide | |
| (4-{[6-(1-methyl(1H-indazol-6-yl))-2-morpholin-4-ylpyrimidin-4- | C |
| yl]amino}phenyl)-N-(methylcyclopropyl)carboxamide | |
| (4-{[6-(1-cyclopropylbenzimidazol-6-yl)-2-morpholin-4-ylpyrimidin-4- | A |
| yl]amino}phenyl)-N-methylcarboxamide | |
| [4-({6-[1-(cyclopropylmethyl)benzimidazol-6-yl]-2-morpholin-4-ylpyrimidin- | A |
| 4-yl}amino)phenyl]-N-methylcarboxamide | |
| (4-{[6-(1-cyclopropylbenzimidazol-5-yl)-2-morpholin-4-ylpyrimidin-4- | B |
| yl]amino}phenyl)-N-methylcarboxamide | |
| (4-chlorophenyl){2-morpholin-4-yl-6-[1-(2-morpholin-4-ylethyl)(1H-indazol- | C |
| 5-yl)]pyrimidin-4-yl}amine | |
| [3-(5-{6-[(4-chlorophenyl)amino]-2-morpholin-4-ylpyrimidin-4- | B |
| yl}benzimidazolyl)propyl]dimethylamine | |
| [4-({6-[1-(cyclopropylmethyl)benzimidazol-5-yl]-2-morpholin-4-ylpyrimidin- | A |
| 4-yl}amino)phenyl]-N-methylcarboxamide | |
| methyl 4-{[6-(1-methyl(1H-indazol-6-yl))-2-morpholin-4-ylpyrimidin-4- | C |
| yl]amino}benzoate | |
| (4-chlorophenyl){2-morpholin-4-yl-6-[1-(2-morpholin-4-ylethyl)(1H-indazol- | B |
| 6-yl)]pyrimidin-4-yl}amine | |
| N-methyl[4-({2-morpholin-4-yl-6-[1-(2-morpholin-4-ylethyl)(1H-indazol-5- | B |
| yl)]pyrimidin-4-yl}amino)phenyl]carboxamide | |
| N-cyclopropyl(4-{[6-(1-methyl(1H-indazol-6-yl))-2-morpholin-4-ylpyrimidin- | C |
| 4-yl]amino}phenyl)carboxamide | |
| (4-chlorophenyl)[6-(1-cyclobutylbenzimidazol-6-yl)-2-morpholin-4- | B |
| ylpyrimidin-4-yl]amine | |
| (4-chlorophenyl)[6-(1-cyclobutyl(1H-indazol-6-yl))-2-morpholin-4- | C |
| ylpyrimidin-4-yl]amine | |
| (4-chlorophenyl){2-morpholin-4-yl-6-[1-(3-pyrrolidinylpropyl)(1H-indazol-6- | C |
| yl)]pyrimidin-4-yl}amine | |
| [6-(1,2-dimethylbenzimidazol-6-yl)-2-morpholin-4-ylpyrimidin-4-yl](4- | B |
| chlorophenyl)amine | |
| (4-chlorophenyl){6-[1-(2-methoxyethyl)(1H-indazol-6-yl)]-2-morpholin-4- | C |
| ylpyrimidin-4-yl}amine | |
| (4-chlorophenyl){2-morpholin-4-yl-6-[1-(3-pyrrolidinylpropyl)benzimidazol- | B |
| 6-yl]pyrimidin-4-yl}amine | |
| [2-(6-{6-[(4-chlorophenyl)amino]-2-morpholin-4-ylpyrimidin-4-yl}(1H- | B |
| indazolyl))ethyl]dimethylamine | |
| [2-(6-{6-[(4-chlorophenyl)amino]-2-morpholin-4-ylpyrimidin-4-yl}(2H- | B |
| indazol-2-yl))ethyl]dimethylamine | |
| [3-(6-{6-[(4-chlorophenyl)amino]-2-morpholin-4-ylpyrimidin-4-yl}(1H- | C |
| indazolyl))propyl]dimethylamine | |
| (4-chlorophenyl){2-morpholin-4-yl-6-[1-(2-pyrrolidinylethyl)(1H-indazol-6- | C |
| yl)]pyrimidin-4-yl}amine | |
| (4-{[6-(1-cyclopropyl(1H-indazol-6-yl))-2-morpholin-4-ylpyrimidin-4- | A |
| yl]amino}phenyl)-N-methylcarboxamide | |
| [4-({6-[1-(cyclopropylmethyl)(1H-indazol-6-yl)]-2-morpholin-4-ylpyrimidin- | A |
| 4-yl}amino)phenyl]-N-methylcarboxamide | |
| (4-{[6-(1,2-dimethylbenzimidazol-6-yl)-2-morpholin-4-ylpyrimidin-4- | A |
| yl]amino}phenyl)-N-methylcarboxamide | |
| [4-({6-[1-(2-methoxyethyl)(1H-indazol-6-yl)]-2-morpholin-4-ylpyrimidin-4- | A |
| yl}amino)phenyl]-N-methylcarboxamide | |
| N-methyl[4-({2-morpholin-4-yl-6-[1-(2-morpholin-4-ylethyl)(1H-indazol-6- | B |
| yl)]pyrimidin-4-yl}amino)phenyl]carboxamide | |
| [6-(1,3-dimethyl(1H-indazol-6-yl))-2-morpholin-4-ylpyrimidin-4-yl](4- | B |
| chlorophenyl)amine | |
| N-(cyclopropylmethyl)(4-{[6-(1-methylbenzimidazol-6-yl)-2-morpholin-4- | B |
| ylpyrimidin-4-yl]amino}phenyl)carboxamide | |
| N,N-dimethyl(4-{[6-(1-methyl(1H-indazol-6-yl))-2-morphlin-4-ylpyrimidin- | B |
| 4-yl]amino}phenyl)carboxamide | |
| methyl 4-({6-[1-(2-methoxyethyl)(1H-indazol-6-yl)]-2-morpholin-4- | B |
| ylpyrimidin-4-yl}amino)benzoate | |
| [3-(6-{6-[(4-chlorophenyl)amino]-2-morpholin-4-ylpyrimidin-4- | B |
| yl}benzimidazolyl)propyl]dimethylamine | |
| (4-{[6-(1-cyclobutyl(1H-indazol-6-yl))-2-morpholin-4-ylpyrimidin-4- | A |
| yl]amino}phenyl)-N-methylcarboxamide | |
| (4-chlorophenyl){2-morpholin-4-yl-6-[1-(3-morpholin-4-ylpropyl)(1H- | C |
| indazol-6-yl)]pyrimidin-4-yl}amine | |
| N-methyl[4-({2-morpholin-4-yl-6-[1-(3-pyrrolidinylpropyl)(1H-indazol-6- | A |
| yl)]pyrimidin-4-yl}amino)phenyl]carboxamide | |
| (4-chlorophenyl){6-[1-(2-methoxyethyl)benzimidazol-6-yl]-2-morpholin-4- | B |
| ylpyrimidin-4-yl}amine | |
| N-cyclopropyl(4-{[6-(1-methylbenzimidazol-6-yl)-2-morpholin-4- | C |
| ylpyrimidin-4-yl]amino}phenyl)carboxamide | |
| 4-{[6-(1-methyl(1H-indazol-6-yl))-2-morpholin-4-ylpyrimidin-4- | B |
| yl]amino}benzamide | |
| [2-(6-{6-[(4-chlorophenyl)amino]-2-morpholin-4-ylpyrimidin-4- | B |
| yl}benzimidazolyl)ethyl]dimethylamine | |
| (4-chlorophenyl){2-morpholin-4-yl-6-[1-(2-pyrrolidinylethyl)benzimidazol-6- | B |
| yl]pyrimidin-4-yl}amine | |
| [4-({6-[1-(2-methoxyethyl)benzimidazol-6-yl]-2-morpholin-4-ylpyrimidin-4- | A |
| yl}amino)phenyl]-N-methylcarboxamide | |
| (4-chlorophenyl){2-morpholin-4-yl-6-[1-(2-morpholin-4- | A |
| ylethyl)benzimidazol-6-yl]pyrimidin-4-yl}amine | |
| (4-chlorophenyl){2-morpholin-4-yl-6-[1-(3-morpholin-4- | B |
| ylpropyl)benzimidazol-6-yl]pyrimidin-4-yl}amine | |
| N-methyl[4-({2-morpholin-4-yl-6-[1-(3-morpholin-4-ylpropyl)(1H-indazol-6- | A |
| yl)]pyrimidin-4-yl}amino)phenyl]carboxamide | |
| 4-{[6-(1-methylbenzimidazol-6-yl)-2-morpholin-4-ylpyrimidin-4- | B |
| yl]amino}benzamide | |
| (4-{[6-(1-cyclobutylbenzimidazol-6-yl)-2-morpholin-4-ylpyrimidin-4- | A |
| yl]amino}phenyl)-N-methylcarboxamide | |
| N-methyl[4-({2-morpholin-4-yl-6-[1-(2-morpholin-4-ylethyl)benzimidazol-6- | B |
| yl]pyrimidin-4-yl}amino)phenyl]carboxamide | |
| {4-[(6-{1-[2-(dimethylamino)ethyl](1H-indazol-6-yl)}-2-morpholin-4- | B |
| ylpyrimidin-4-yl)amino]phenyl}-N-methylcarboxamide | |
| {4-[(6-{2-[2-(dimethylamino)ethyl](2H-indazol-6-yl)}-2-morpholin-4- | A |
| ylpyrimidin-4-yl)amino]phenyl}-N-methylcarboxamide | |
| (4-chlorophenyl){2-morpholin-4-yl-6-[1-(3-morpholin-4-ylpropyl)(1H- | C |
| indazol-5-yl)]pyrimidin-4-yl}amine | |
| methyl 4-({2-morpholin-4-yl-6-[1-(3-morpholin-4-ylpropyl)(1H-indazol-6- | B |
| yl)]pyrimidin-4-yl}amino)benzoate | |
| (4-{[6-(1-cyclopropyl(1H-indazol-5-yl))-2-morpholin-4-ylpyrimidin-4- | C |
| yl]amino}phenyl)-N-methylcarboxamide | |
| (4-{[6-(1,3-dimethyl(1H-indazol-6-yl))-2-morpholin-4-ylpyrimidin-4- | A |
| yl]amino}phenyl)-N-methylcarboxamide | |
| (4-chlorophenyl){2-morpholin-4-yl-6-[1-(2-morpholin-4- | B |
| ylethyl)benzimidazol-5-yl]pyrimidin-4-yl}amine | |
| N-methyl[4-({2-morpholin-4-yl-6-[1-(3-morpholin-4-ylpropyl)(1H-indazol-5- | C |
| yl)]pyrimidin-4-yl}amino)phenyl]carboxamide | |
| {4-[(6-{1-[2-(dimethylamino)ethyl]benzimidazol-6-yl}-2-morpholin-4- | A |
| ylpyrimidin-4-yl)amino]phenyl}-N-methylcarboxamide | |
| N-methyl[4-({2-morpholin-4-yl-6-[1-(2-pyrrolidinylethyl)benzimidazol-6- | C |
| yl]pyrimidin-4-yl}amino)phenyl]carboxamide | |
| (4-chlorophenyl)[6-(1-cyclopropyl-2-methylbenzimidazol-6-yl)-2-morpholin- | B |
| 4-ylpyrimidin-4-yl]amine | |
| (4-chlorophenyl)[6-(1-cyclobutyl-2-methylbenzimidazol-6-yl)-2-morpholin-4- | C |
| ylpyrimidin-4-yl]amine | |
| N-ethyl(4-{[6-(1-methylbenzimidazol-6-yl)-2-morpholin-4-ylpyrimidin-4- | C |
| yl]amino}phenyl)carboxamide | |
| methyl 4-({6-[1-(2-methoxyethyl)benzimidazol-6-yl]-2-morpholin-4- | A |
| ylpyrimidin-4-yl}amino)benzoate | |
| [2-(5-{6-[(4-chlorophenyl)amino]-2-morpholin-4-ylpyrimidin-4- | C |
| yl}benzimidazolyl)ethyl]dimethylamine | |
| (4-chlorophenyl){2-morpholin-4-yl-6-[1-(2-pyrrolidinylethyl)benzimidazol-5- | C |
| yl]pyrimidin-4-yl}amine | |
| (4-chlorophenyl){2-morpholin-4-yl-6-[1-(3-morpholin-4- | C |
| ylpropyl)benzimidazol-5-yl]pyrimidin-4-yl}amine | |
| N-methyl[4-({2-morpholin-4-yl-6-[1-(3-morpholin-4-ylpropyl)benzimidazol- | C |
| 6-yl]pyrimidin-4-yl}amino)phenyl]carboxamide | |
| {4-[(6-{1-[2-(dimethylamino)ethyl]benzimidazol-5-yl}-2-morpholin-4- | C |
| ylpyrimidin-4-yl)amino]phenyl}-N-methylcarboxamide | |
| 4-({6-[1-(2-methoxyethyl)benzimidazol-6-yl]-2-morpholin-4-ylpyrimidin-4- | C |
| yl}amino)benzoic acid | |
| (4-{[6-(1-cyclopropyl-2-methylbenzimidazol-6-yl)-2-morpholin-4- | A |
| ylpyrimidin-4-yl]amino}phenyl)-N-methylcarboxamide | |
| [4-({6-[1-(cyclopropylmethyl)-2-methylbenzimidazol-6-yl]-2-morpholin-4- | A |
| ylpyrimidin-4-yl}amino)phenyl]-N-methylcarboxamide | |
| 4-{[6-(1-methylbenzimidazol-6-yl)-2-morpholin-4-ylpyrimidin-4- | C |
| yl]amino}benzenecarbonitrile | |
| N-ethyl(4-{[6-(1-methyl(1H-indazol-6-yl))-2-morpholin-4-ylpyrimidin-4- | B |
| yl]amino}phenyl)carboxamide | |
| (4-{[6-(1-methyl(1H-indazol-6-yl))-2-morpholin-4-ylpyrimidin-4- | B |
| yl]amino}phenyl)-N-(methylethyl)carboxamide | |
| N-methyl[4-({2-morpholin-4-yl-6-[1-(2-pyrrolidinylethyl)(1H-indazol-6- | B |
| yl)]pyrimidin-4-yl}amino)phenyl]carboxamide | |
| N-methyl[4-({2-morpholin-4-yl-6-[1-(3-morpholin-4-ylpropyl)benzimidazol- | C |
| 5-yl]pyrimidin-4-yl}amino)phenyl]carboxamide | |
| (4-chlorophenyl)[6-(1-cyclobutyl-2-methylbenzimidazol-5-yl)-2-morpholin-4- | C |
| ylpyrimidin-4-yl]amine | |
| (4-chlorophenyl){6-[1-methyl-3-(morpholin-4-ylmethyl)(1H-indazol-6-yl)]-2- | C |
| morpholin-4-ylpyrimidin-4-yl}amine | |
| (4-{[6-(1-methylbenzimidazol-6-yl)-2-morpholin-4-ylpyrimidin-4- | C |
| yl]amino}phenyl)-N-(methylethyl)carboxamide | |
| 4-{[6-(1-methyl(1H-indazol-6-yl))-2-morpholin-4-ylpyrimidin-4- | C |
| yl]amino}benzenecarbonitrile | |
| 4-(6-{6-[(4-chlorophenyl)amino]-2-morpholin-4-ylpyrimidin-4-yl}-1-methyl- | C |
| 1H-indazol-3-yl)butan-1-ol | |
| N-methyl[4-({2-morpholin-4-yl-6-[1-(2-morpholin-4-ylethyl)benzimidazol-5- | B |
| yl]pyrimidin-4-yl}amino)phenyl]carboxamide | |
| (4-chlorophenyl){6-[2-methyl-1-(3-morpholin-4-ylpropyl)benzimidazol-6-yl]- | C |
| 2-morpholin-4-ylpyrimidin-4-yl}amine | |
| (4-chlorophenyl)[6-(1-cyclopropyl-3-methyl(1H-indazol-6-yl))-2-morpholin- | C |
| 4-ylpyrimidin-4-yl]amine | |
| N-methyl[4-({2-morpholin-4-yl-6-[1-(3-pyrrolidinylpropyl)benzimidazol-5- | B |
| yl]pyrimidin-4-yl}amino)phenyl]carboxamide | |
| (4-chlorophenyl){6-[2-methyl-1-(2-morpholin-4-ylethyl)benzimidazol-6-yl]-2- | B |
| morpholin-4-ylpyrimidin-4-yl}amine | |
| {6-[3-chloro-1-(3-morpholin-4-ylpropyl)(1H-indazol-6-yl)]-2-morpholin-4- | C |
| ylpyrimidin-4-yl}(4-chlorophenyl)amine | |
| (4-chlorophenyl){6-[2-methyl-1-(2-pyrrolidinylethyl)benzimidazol-6-yl]-2- | C |
| morpholin-4-ylpyrimidin-4-yl}amine | |
| (4-chlorophenyl){6-[1-methyl-3-(3-pyrrolidinylpropyl)(1H-indazol-6-yl)]-2- | B |
| morpholin-4-ylpyrimidin-4-yl}amine | |
| (4-chlorophenyl){6-[1-methyl-3-(3-morpholin-4-ylpropyl)(1H-indazol-6-yl)]- | C |
| 2-morpholin-4-ylpyrimidin-4-yl}amine | |
| 3-(6-{6-[(4-chlorophenyl)amino]-2-morpholin-4-ylpyrimidin-4-yl}-1-methyl- | B |
| 1H-indazol-3-yl)propan-1-ol | |
| (4-{[6-(1-cyclobutyl-2-methylbenzimidazol-6-yl)-2-morpholin-4-ylpyrimidin- | A |
| 4-yl]amino}phenyl)-N-methylcarboxamide | |
| [3-(6-{6-[(4-chlorophenyl)amino]-2-morpholin-4-ylpyrimidin-4-yl}-2- | B |
| methylbenzimidazolyl)propyl]dimethylamine | |
| (4-chlorophenyl){6-[2-methyl-1-(3-pyrrolidinylpropyl)benzimidazol-6-yl]-2- | B |
| morpholin-4-ylpyrimidin-4-yl}amine | |
| {4-[(6-{1-[3-(dimethylamino)propyl]benzimidazol-6-yl}-2-morpholin-4- | C |
| ylpyrimidin-4-yl)amino]phenyl}-N-methylcarboxamide | |
| [2-(6-{6-[(4-chlorophenyl)amino]-2-morpholin-4-ylpyrimidin-4-yl}-2- | C |
| methylbenzimidazolyl)ethyl]dimethylamine | |
| (4-chlorophenyl){6-[1-methyl-3-(2-pyrrolidinylethyl)(1H-indazol-6-yl)]-2- | B |
| morpholin-4-ylpyrimidin-4-yl}amine | |
| (4-chlorophenyl){6-[1-methyl-3-(pyrrolidinylmethyl)(1H-indazol-6-yl)]-2- | B |
| morpholin-4-ylpyrimidin-4-yl}amine | |
| [6-(1-methylbenzimidazol-6-yl)-2-morpholin-4-ylpyrimidin-4-yl]phenylamine | C |
| (4-methoxyphenyl)[6-(1-methylbenzimidazol-6-yl)-2-morpholin-4- | C |
| ylpyrimidin-4-yl]amine | |
| (4-{[6-(1-methylbenzimidazol-6-yl)-2-morpholin-4-ylpyrimidin-4- | C |
| yl]amino}phenyl)(methylsulfonyl)amine | |
| (4-chlorophenyl)(6-{1-methyl-3-[(2-morpholin-4-ylethyl)amino](1H-indazol- | B |
| 6-yl)}-2-morpholin-4-ylpyrimidin-4-yl)amine | |
| (4-bromophenyl)[6-(1-methylbenzimidazol-6-yl)-2-morpholin-4-ylpyrimidin- | B |
| 4-yl]amine | |
| dimethyl(4-{[6-(1-methylbenzimidazol-6-yl)-2-morpholin-4-ylpyrimidin-4- | C |
| yl]amino}phenyl)amine | |
| (4-chlorophenyl){6-[3-methyl-1-(2-morpholin-4-ylethyl)(1H-indazol-6-yl)]-2- | B |
| morpholin-4-ylpyrimidin-4-yl}amine | |
| (4-chlorophenyl)[6-(1-cyclobutyl-3-methyl(1H-indazol-6-yl))-2-morpholin-4- | C |
| ylpyrimidin-4-yl]amine | |
| [2-(6-{6-[(4-chlorophenyl)amino]-2-morpholin-4-ylpyrimidin-4-yl}-1- | B |
| methyl(1H-indazol-3-yl))ethyl]dimethylamine | |
| (4-chlorophenyl){6-[1-methyl-3-(2-morpholin-4-ylethyl)(1H-indazol-6-yl)]-2- | B |
| morpholin-4-ylpyrimidin-4-yl}amine | |
| (4-fluorophenyl)[6-(1-methylbenzimidazol-6-yl)-2-morpholin-4-ylpyrimidin- | C |
| 4-yl]amine | |
| [6-(3-chloro-1-methyl(1H-indazol-6-yl))-2-morpholin-4-ylpyrimidin-4-yl](4- | C |
| chlorophenyl)amine | |
| (4-chlorophenyl)(6-{1-methyl-3-[(3-morpholin-4-ylpropyl)amino](1H- | B |
| indazol-6-yl)}-2-morpholin-4-ylpyrimidin-4-yl)amine | |
| N-(4-{[6-(1-methylbenzimidazol-6-yl)-2-morpholin-4-ylpyrimidin-4- | B |
| yl]amino}phenyl)acetamide | |
| (4-chlorophenyl){6-[1-(cyclopropylmethyl)-3-methyl(1H-indazol-6-yl)]-2- | C |
| morpholin-4-ylpyrimidin-4-yl}amine | |
| 4-({2-morpholin-4-yl-6-[1-(2-morpholin-4-ylethyl)benzimidazol-6- | C |
| yl]pyrimidin-4-yl}amino)benzamide | |
| 4-{[6-(1-methylbenzimidazol-6-yl)-2-morpholin-4-ylpyrimidin-4- | C |
| yl]amino}benzenesulfonamide | |
| (4-chlorophenyl){6-[1-methyl-2-(morpholin-4-ylmethyl)benzimidazol-6-yl]-2- | B |
| morpholin-4-ylpyrimidin-4-yl}amine | |
| N-(4-{[6-(1-methyl(1H-indazol-6-yl))-2-morpholin-4-ylpyrimidin-4- | B |
| yl]amino}phenyl)acetamide | |
| (tert-butoxy)-N-(4-{[6-(1-methylbenzimidazol-6-yl)-2-morpholin-4- | C |
| ylpyrimidin-4-yl]amino}phenyl)carboxamide | |
| (4-chlorophenyl)[6-(1-methyl-2-pyrrolidinylbenzimidazol-6-yl)-2-morpholin- | C |
| 4-ylpyrimidin-4-yl]amine | |
| [2-(6-{6-[(4-chlorophenyl)amino]-2-morpholin-4-ylpyrimidin-4-yl}-3- | C |
| methyl(1H-indazoly))ethyl]dimethylamine | |
| {6-[3-(3-aminopropyl)-1-methyl(1H-indazol-6-yl)]-2-morpholin-4- | C |
| ylpyrimidin-4-yl}(4-chlorophenyl)amine | |
| [2-(6-{6-[(4-chlorophenyl)amino]-2-morpholin-4-ylpyrimidin-4-yl}-3- | C |
| methyl(2H-indazol-2-yl))ethyl]dimethylamine | |
| 1-{[6-(1-methyl(1H-indazol-6-yl))-2-morpholin-4-ylpyrimidin-4-yl]amino}-4- | C |
| (methylsulfonyl)benzene | |
| 4-{[6-(1-methyl(1H-indazol-6-yl))-2-morpholin-4-ylpyrimidin-4- | C |
| yl]amino}benzenesulfonamide | |
| (4-{[6-(1-methyl(1H-indazol-6-yl))-2-morpholin-4-ylpyrimidin-4- | B |
| yl]amino}phenyl)(methylsulfonyl)amine | |
| (4-chlorophenyl){6-[3-methyl-1-(2-pyrrolidinylethyl)(1H-indazol-6-yl)]-2- | C |
| morpholin-4-ylpyrimidin-4-yl}amine | |
| (4-chlorophenyl)(6-{1-methyl-3-[(4-methylpiperazinyl)methyl](1H-indazol-6- | B |
| yl)}-2-morpholin-4-ylpyrimidin-4-yl)amine | |
| methyl[(4-{[6-(1-methyl(1H-indazol-6-yl))-2-morpholin-4-ylpyrimidin-4- | C |
| yl]amino}phenyl)sulfonyl]amine | |
| [6-(1-methyl(1H-indazol-6-yl))-2-morpholin-4-ylpyrimidin-4-yl][4-(5- | A |
| methyl(4H-1,2,4-triazol-3-yl))phenyl]amine | |
| {2-[(6-{6-[(4-chlorophenyl)amino]-2-morpholin-4-ylpyrimidin-4-yl}-1- | B |
| methyl(1H-indazol-3-yl))amino]ethyl}dimethylamine | |
| [3-(6-{6-[(4-chlorophenyl)amino]-2-morpholin-4-ylpyrimidin-4-yl}-3- | B |
| methyl(1H-indazolyl))propyl]dimethylamine | |
| (4-chlorophenyl){6-[1-methyl-2-(pyrrolidinylmethyl)benzimidazol-6-yl]-2- | C |
| morpholin-4-ylpyrimidin-4-yl}amine | |
| (4-chlorophenyl)(6-{1-methyl-3-[(2-pyrrolidinylethyl)amino](1H-indazol-6- | B |
| yl)}-2-morpholin-4-ylpyrimidin-4-yl)amine | |
| (4-chlorophenyl)[6-(1-methyl-3-morpholin-4-yl(1H-indazol-6-yl))-2- | C |
| morpholin-4-ylpyrimidin-4-yl]amine | |
| (4-chlorophenyl){6-[1-methyl-2-(2-morpholin-4-ylethyl)benzimidazol-6-yl]-2- | A |
| morpholin-4-ylpyrimidin-4-yl}amine | |
| [6-(1-methyl(1H-indazol-6-yl))-2-morpholin-4-ylpyrimidin-4-yl]phenylamine | C |
| (4-methoxyphenyl)[6-(1-methyl(1H-indazol-6-yl))-2-morpholin-4- | C |
| ylpyrimidin-4-yl]amine | |
| (4-chlorophenyl){6-[1-methyl-2-(2-pyrrolidinylethyl)benzimidazol-6-yl]-2- | A |
| morpholin-4-ylpyrimidin-4-yl}amine | |
| (4-chlorophenyl)[6-(1-methylpyrazolo[4,5-b]pyridin-6-yl)-2-morpholin-4- | B |
| ylpyrimidin-4-yl]amine | |
| (4-chlorophenyl){6-[3-methyl-1-(3-pyrrolidinylpropyl)(1H-indazol-6-yl)]-2- | B |
| morpholin-4-ylpyrimidin-4-yl}amine | |
| (4-chlorophenyl){6-[3-methyl-1-(3-morpholin-4-ylpropyl)(1H-indazol-6-yl)]- | C |
| 2-morpholin-4-ylpyrimidin-4-yl}amine | |
| (4-chlorophenyl)(6-{2-[(2-methoxyethyl)amino]-1-methylbenzimidazol-6-yl}- | B |
| 2-morpholin-4-ylpyrimidin-4-yl)amine | |
| 1-{[6-(1-methyl(1H-indazol-6-yl))-2-morpholin-4-ylpyrimidin-4-yl]amino}-4- | C |
| (methylsulfinyl)benzene | |
| (4-chlorophenyl)(6-{1-methyl-3-[(3-pyrrolidinylpropyl)amino](1H-indazol-6- | C |
| yl)}-2-morpholin-4-ylpyrimidin-4-yl)amine | |
| (4-chlorophenyl){6-[1-methyl-2-(3-morpholin-4-ylpropyl)benzimidazol-6-yl]- | C |
| 2-morpholin-4-ylpyrimidin-4-yl}amine | |
| (4-chlorophenyl){6-[1-methyl-3-(4-methylpiperazinyl)(1H-indazol-6-yl)]-2- | C |
| morpholin-4-ylpyrimidin-4-yl}amine | |
| [2-(6-{6-[(4-chlorophenyl)amino]-2-morpholin-4-ylpyrimidin-4-yl} | A |
| methylbenzimidazol-2-yl)ethyl]dimethylamine | |
| (4-chlorophenyl)[6-(1-methyl-3-pyrrolidinyl(1H-indazol-6-yl))-2-morpholin- | C |
| 4-ylpyrimidin-4-yl]amine | |
| [6-(1,3-dimethylpyrazolo[4,5-b]pyridin-6-yl)-2-morpholin-4-ylpyrimidin-4- | B |
| yl](4-chlorophenyl)amine | |
| (4-chlorophenyl)[6-(1-methylpyrazolo[4,5-e]pyridin-6-yl)-2-morpholin-4- | C |
| ylpyrimidin-4-yl]amine | |
| 1-acetyl-4-{[6-(1-methyl(1H-indazol-6-yl))-2-morpholin-4-ylpyrimidin-4- | B |
| yl]amino}benzene | |
| (4-(4H-1,2,4-triazol-3-yl)phenyl)[6-(1-methyl(1H-indazol-6-yl))-2-morpholin- | A |
| 4-ylpyrimidin-4-yl]amine | |
| [6-(1,3-dimethylpyrazolo[4,5-e]pyridin-6-yl)-2-morpholin-4-ylpyrimidin-4- | C |
| yl](4-chlorophenyl)amine | |
| 6-{6-[(4-chlorophenyl)amino]-2-morpholin-4-ylpyrimidin-4-yl}-1-methyl-3- | A |
| hydrobenzimidazol-2-one | |
| (4-chlorophenyl)(6-{1-methyl-3-[(4-pyrrolidinylbutyl)amino](1H-indazol-6- | B |
| yl)}-2-morpholin-4-ylpyrimidin-4-yl)amine | |
| [6-(1-methyl(1H-indazol-6-yl))-2-morpholin-4-ylpyrimidin-4-yl](4-(1,3- | C |
| oxazolin-2-yl)phenyl)amine | |
| (4-chlorophenyl)(6-{1-[2-(4-methylpiperazinyl)ethyl](1H-indazol-6-yl)}-2- | B |
| morpholin-4-ylpyrimidin-4-yl)amine | |
| [6-(1-methyl(1H-indazol-6-yl))-2-morpholin-4-ylpyrimidin-4-yl](4-(1,3- | B |
| oxazol-2-yl)phenyl)amine | |
| 1H-indazol-5-yl[6-(1-methyl(1H-indazol-6-yl))-2-morpholin-4-ylpyrimidin-4- | C |
| yl]amine | |
| (4-chlorophenyl)(6-{1-[3-(4-methylpiperazinyl)propyl](1H-indazol-6-yl)}-2- | B |
| morpholin-4-ylpyrimidin-4-yl)amine | |
| (6-{6-[(4-chlorophenyl)amino]-2-morpholin-4-ylpyrimidin-4-yl}-1- | C |
| methyl(1H-indazol-3-yl))dimethylamine | |
| (4-chlorophenyl){6-[2-methyl-1-(2-pyrrolidinylethyl)benzimidazol-5-yl]-2- | B |
| morpholin-4-ylpyrimidin-4-yl}amine | |
| (4-chlorophenyl){6-[2-methyl-1-(2-morpholin-4-ylethyl)benzimidazol-5-yl]-2- | C |
| morpholin-4-ylpyrimidin-4-yl}amine | |
| (4-chlorophenyl){6-[2-(2-methoxyethyl)-1-methylbenzimidazol-6-yl]-2- | B |
| morpholin-4-ylpyrimidin-4-yl}amine | |
| [6-(1-methyl(1H-indazol-6-yl))-2-morpholin-4-ylpyrimidin-4-yl](3-(1,3- | C |
| oxazol-2-yl)phenyl)amine | |
| (4-chlorophenyl)[5-methyl-6-(1-methyl(1H-indazol-6-yl))-2-morpholin-4- | C |
| ylpyrimidin-4-yl]amine | |
| (1-methyl(1H-indazol-5-yl))[6-(1-methyl(1H-indazol-6-yl))-2-morpholin-4- | C |
| ylpyrimidin-4-yl]amine | |
| [6-(1-methyl(1H-indazol-6-yl))-2-morpholin-4-ylpyrimidin-4-yl](2- | C |
| methyl(2H-indazol-5-yl))amine | |
| [6-(1,3-dimethyl(1H-indazol-6-yl))-2-morpholin-4-ylpyrimidin-4-yl]indol-5- | C |
| ylamine | |
| (4-(4H-1,2,4-triazol-3-yl)phenyl)[6-(1,3-dimethyl(1H-indazol-6-yl))-2- | A |
| morpholin-4-ylpyrimidin-4-yl]amine | |
| [6-(1-methyl(1H-indazol-6-yl))-2-morpholin-4-ylpyrimidin-4-yl](4-pyrazol-5- | A |
| ylphenyl)amine | |
| [6-(1,3-dimethyl(1H-indazol-6-yl))-2-morpholin-4-ylpyrimidin-4-yl][4-(5- | A |
| methyl(4H-1,2,4-triazol-3-yl))phenyl]amine | |
| 1H-indazol-6-yl[6-(1-methyl(1H-indazol-6-yl))-2-morpholin-4-ylpyrimidin-4- | C |
| yl]amine | |
| 1H-indazol-6-yl[6-(1,3-dimethyl(1H-indazol-6-yl))-2-morpholin-4- | C |
| ylpyrimidin-4-yl]amine | |
| [4-(5-methyl(1,2,4-oxadiazol-3-yl))phenyl][6-(1-methyl(1H-indazol-6-yl))-2- | C |
| morpholin-4-ylpyrimidin-4-yl]amine | |
| [6-(1,3-dimethyl(1H-indazol-6-yl))-2-morpholin-4-ylpyrimidin-4-yl](4-(1,3- | B |
| oxazol-2-yl)phenyl)amine | |
| [4-(5-methyl(4H-1,2,4-triazol-3-yl))phenyl]{2-morpholin-4-yl-6-[1-(2- | A |
| morpholin-4-ylethyl)(1H-indazol-6-yl)]pyrimidin-4-yl}amine | |
| (4-imidazol-2-ylphenyl)[6-(1-methyl(1H-indazol-6-yl))-2-morpholin-4- | A |
| ylpyrimidin-4-yl]amine | |
| [6-(1-methyl(1H-indazol-6-yl))-2-morpholin-4-ylpyrimidin-4-yl][4-(1- | B |
| methylimidazol-2-yl)phenyl]amine | |
| [6-(1,3-dimethyl(1H-indazol-6-yl))-2-morpholin-4-ylpyrimidin-4-yl](4- | A |
| imidazol-2-ylphenyl)amine | |
| [6-(1,3-dimethyl(1H-indazol-6-yl))-2-morpholin-4-ylpyrimidin-4-yl][4-(1- | A |
| methylimidazol-2-yl)phenyl]amine | |
| [6-(1,3-dimethyl(1H-indazol-6-yl))-2-morpholin-4-ylpyrimidin-4-yl](4-(1,3- | C |
| oxazolin-2-yl)phenyl)amine | |
| [6-(1,3-dimethyl(1H-indazol-6-yl))-2-morpholin-4-ylpyrimidin-4-yl](2- | C |
| methyl(2H-indazol-5-yl))amine | |
| [4-(5-methyl(4H-1,2,4-triazol-3-yl))phenyl]{6-[1-methyl-3-(2-morpholin-4- | A |
| ylethyl)(1H-indazol-6-yl)]-2-morpholin-4-ylpyrimidin-4-yl}amine | |
| [6-(1,3-dimethylpyrazolo[5,4-d]pyridin-6-yl)-2-morpholin-4-ylpyrimidin-4- | A |
| yl](4-chlorophenyl)amine | |
| [6-(1-methyl(1H-indazol-6-yl))-2-morpholin-4-ylpyrimidin-4-yl][4-(1- | B |
| methylpyrazol-3-yl)phenyl]amine | |
| (1-methyl(1H-indazol-6-yl))[6-(1-methyl(1H-indazol-6-yl))-2-morpholin-4- | C |
| ylpyrimidin-4-yl]amine | |
| [6-(1-methyl(1H-indazol-6-yl))-2-morpholin-4-ylpyrimidin-4-yl](4-(1,3- | A |
| oxazol-4-yl)phenyl)amine | |
| [6-(1,3-dimethyl(1H-indazol-6-yl))-2-morpholin-4-ylpyrimidin-4-yl](4-(1,3- | A |
| oxazol-4-yl)phenyl)amine | |
| [6-(1-methyl(1H-indazol-6-yl))-2-morpholin-4-ylpyrimidin-4-yl](2- | C |
| methyl(2H-indazol-6-yl))amine | |
| [6-(1,3-dimethyl(1H-indazol-6-yl))-2-morpholin-4-ylpyrimidin-4-yl](2- | C |
| methyl(2H-indazol-6-yl))amine | |
| [6-(1-methyl(1H-indazol-6-yl))-2-morpholin-4-ylpyrimidin-4-yl][4-(1- | B |
| methylimidazol-4-yl)phenyl]amine | |
| [6-(1,3-dimethyl(1H-indazol-6-yl))-2-morpholin-4-ylpyrimidin-4-yl][4-(1- | C |
| methylimidazol-4-yl)phenyl]amine | |
| [6-(1-methyl(1H-indazol-6-yl))-2-morpholin-4-ylpyrimidin-4-yl](1- | B |
| methylbenzimidazol-6-yl)amine | |
| [6-(1,3-dimethyl(1H-indazol-6-yl))-2-morpholin-4-ylpyrimidin-4-yl](1- | C |
| methylbenzimidazol-6-yl)amine | |
| [6-(1-methyl(1H-indazol-6-yl))-2-morpholin-4-ylpyrimidin-4-yl](4-(1,2,4- | B |
| oxadiazol-3-yl)phenyl)amine | |
| [4-(5-methyl(4H-1,2,4-triazol-3-yl))phenyl]{6-[3-methyl-1-(2-morpholin-4- | A |
| ylethyl)(1H-indazol-6-yl)]-2-morpholin-4-ylpyrimidin-4-yl}amine | |
| [6-(1,3-dimethyl(1H-indazol-6-yl))-2-morpholin-4-ylpyrimidin-4-yl][4-(1- | C |
| methylpyrazol-3-yl)phenyl]amine | |
| [6-(1-methyl(1H-indazol-6-yl))-2-morpholin-4-ylpyrimidin-4-yl](1- | C |
| methylindol-5-yl)amine | |
| (4-chlorophenyl)[2-morpholin-4-yl-6-(1-oxetan-3-ylbenzimidazol-6- | A |
| yl)pyrimidin-4-yl]amine | |
| (4-chlorophenyl)[2-morpholin-4-yl-6-(1-oxetan-3-ylbenzimidazol-5- | B |
| yl)pyrimidin-4-yl]amine | |
| [6-(1-methyl(1H-indazol-6-yl))-2-morpholin-4-ylpyrimidin-4-yl](1- | A |
| methylindol-6-yl)amine | |
| [6-(1,3-dimethyl(1H-indazol-6-yl))-2-morpholin-4-ylpyrimidin-4-yl](1- | A |
| methylindol-6-yl)amine | |
| {6-[1-(cyclopropylmethyl)-2-methylbenzimidazol-6-yl]-2-morpholin-4- | A |
| ylpyrimidin-4-yl}[4-(5-methyl(4H-1,2,4-triazol-3-yl))phenyl]amine | |
| indol-6-yl[6-(1-methyl(1H-indazol-6-yl))-2-morpholin-4-ylpyrimidin-4- | C |
| yl]amine | |
| [6-(1,3-dimethyl(1H-indazol-6-yl))-2-morpholin-4-ylpyrimidin-4-yl]indol-6- | B |
| ylamine | |
| [6-(1,3-dimethyl(1H-indazol-6-yl))-2-morpholin-4-ylpyrimidin-4-yl](1- | B |
| methylindol-5-yl)amine | |
| [6-(1,3-dimethyl(1H-indazol-6-yl))-2-morpholin-4-ylpyrimidin-4-yl](4- | A |
| pyrazol-5-ylphenyl)amine | |
| [6-(1-methyl(1H-indazol-6-yl))-2-morpholin-4-ylpyrimidin-4-yl]{4-[5- | C |
| (trifluoromethyl)(4H-1,2,4-triazol-3-yl)]phenyl}amine | |
| [6-(1,3-dimethyl(1H-indazol-6-yl))-2-morpholin-4-ylpyrimidin-4-yl](4- | C |
| imidazol-4-ylphenyl)amine | |
| 1H-indazol-5-yl[6-(1,3-dimethyl(1H-indazol-6-yl))-2-morpholin-4- | C |
| ylpyrimidin-4-yl]amine | |
| benzoxazol-6-yl[6-(1-methyl(1H-indazol-6-yl))-2-morpholin-4-ylpyrimidin-4- | C |
| yl]amine | |
| [6-(1,3-dimethyl(1H-indazol-6-yl))-2-morpholin-4-ylpyrimidin-4-yl](4-(1,2,4- | C |
| oxadiazol-5-yl)phenyl)amine | |
| [6-(1,3-dimethyl(1H-indazol-6-yl))-2-morpholin-4-ylpyrimidin-4-yl](4-(1,2,4- | B |
| oxadiazol-3-yl)phenyl)amine | |
| [6-(1,3-dimethyl(1H-indazol-6-yl))-2-morpholin-4-ylpyrimidin-4-yl][4-(1- | B |
| methylpyrrol-2-yl)phenyl]amine | |
| [6-(1-methyl(1H-indazol-6-yl))-2-morpholin-4-ylpyrimidin-4-yl](1- | C |
| methylbenzimidazol-5-yl)amine | |
| [6-(1,3-dimethyl(1H-indazol-6-yl))-2-morpholin-4-ylpyrimidin-4-yl]{4-[5- | C |
| (trifluoromethyl)(4H-1,2,4-triazol-3-yl)]phenyl}amine | |
| (4-(1H-1,2,3-triazol-5-yl)phenyl)[6-(1-methyl(1H-indazol-6-yl))-2-morpholin- | C |
| 4-ylpyrimidin-4-yl]amine | |
| [4-(4-methyl(1,2,4-triazol-3-yl))phenyl][6-(1-methyl(1H-indazol-6-yl))-2- | C |
| morpholin-4-ylpyrimidin-4-yl]amine | |
| (4-chlorophenyl)[6-(2-cyclopropyl-1-methylbenzimidazol-6-yl)-2-morpholin- | B |
| 4-ylpyrimidin-4-yl]amine | |
| (4-imidazol-4-ylphenyl)[6-(1-methyl(1H-indazol-6-yl))-2-morpholin-4- | B |
| ylpyrimidin-4-yl]amine | |
| [4-(5-methyl(4H-1,2,4-triazol-3-yl))phenyl][6-(1-methylpyrazolo[4,5- | A |
| b]pyridin-6-yl)-2-morpholin-4-ylpyrimidin-4-yl]amine | |
| (4-(1H-1,2,3-triazol-5-yl)phenyl)[6-(1,3-dimethyl(1H-indazol-6-yl))-2- | B |
| morpholin-4-ylpyrimidin-4-yl]amine | |
| [6-(1,3-dimethyl(1H-indazol-6-yl))-2-morpholin-4-ylpyrimidin-4-yl][4-(1- | C |
| methylpyrrol-3-yl)phenyl]amine | |
| [6-(1-methyl(1H-indazol-6-yl))-2-morpholin-4-ylpyrimidin-4-yl](2-methyl(2- | C |
| hydrobenzotriazol-5-yl))amine | |
| [6-(1-methyl(1H-indazol-6-yl))-2-morpholin-4-ylpyrimidin-4-yl](1- | C |
| methylbenzotriazol-5-yl)amine | |
| [6-(1,3-dimethyl(1H-indazol-6-yl))-2-morpholin-4-ylpyrimidin-4-yl](2- | C |
| methyl(2-hydrobenzotriazol-5-yl))amine | |
| [6-(1-methyl(1H-indazol-6-yl))-2-morpholin-4-ylpyrimidin-4-yl](1- | C |
| methylbenzotriazol-6-yl)amine | |
| (4-chlorophenyl){5-fluoro-6-[1-methyl-3-(2-morpholin-4-ylethyl)(1H-indazol- | C |
| 6-yl)]-2-morpholin-4-ylpyrimidin-4-yl}amine | |
| [6-(1-methyl(1H-indazol-6-yl))-2-morpholin-4-ylpyrimidin-4-yl](4-pyrrol-2- | C |
| ylphenyl)amine | |
| [6-(1-methyl(1H-indazol-6-yl))-2-morpholin-4-ylpyrimidin-4-yl](4-pyrrol-3- | B |
| ylphenyl)amine | |
| (4-(4H-1,2,4-triazol-3-yl)phenyl)[6-(1-methylpyrazolo[4,5-b]pyridin-6-yl)-2- | A |
| morpholin-4-ylpyrimidin-4-yl]amine | |
| [6-(1,3-dimethyl(1H-indazol-6-yl))-2-morpholin-4-ylpyrimidin-4-yl][4-(5- | A |
| cyclopropyl(4H-1,2,4-triazol-3-yl))phenyl]amine | |
| (1-methylindol-6-yl){6-[1-methyl-3-(2-morpholin-4-ylethyl)(1H-indazol-6- | A |
| yl)]-2-morpholin-4-ylpyrimidin-4-yl}amine | |
| [6-(1,3-dimethyl(1H-indazol-6-yl))-2-morpholin-4-ylpyrimidin-4-yl](4-pyrrol- | B |
| 3-ylphenyl)amine | |
| [6-(1,3-dimethyl(1H-indazol-6-yl))-2-morpholin-4-ylpyrimidin-4-yl](4-pyrrol- | A |
| 2-ylphenyl)amine | |
| [4-(5-cyclopropyl(4H-1,2,4-triazol-3-yl))phenyl][6-(1-methyl(1H-indazol-6- | A |
| yl))-2-morpholin-4-ylpyrimidin-4-yl]amine | |
| indol-6-yl{6-[1-methyl-3-(2-morpholin-4-ylethyl)(1H-indazol-6-yl)]-2- | B |
| morpholin-4-ylpyrimidin-4-yl}amine | |
| (4-fluorophenyl){6-[1-methyl-3-(2-morpholin-4-ylethyl)(1H-indazol-6-yl)]-2- | C |
| morpholin-4-ylpyrimidin-4-yl}amine | |
| (4-bromophenyl){6-[1-methyl-3-(2-morpholin-4-ylethyl)(1H-indazol-6-yl)]-2- | B |
| morpholin-4-ylpyrimidin-4-yl}amine | |
| {6-[1-methyl-3-(2-morpholin-4-ylethyl)(1H-indazol-6-yl)]-2-morpholin-4- | B |
| ylpyrimidin-4-yl}(4-methylphenyl)amine | |
| 4-({6-[1-methyl-3-(2-morpholin-4-ylethyl)(1H-indazol-6-yl)]-2-morpholin-4- | C |
| ylpyrimidin-4-yl}amino)benzenecarbonitrile | |
| 1H-indazol-6-yl{6-[1-methyl-3-(2-morpholin-4-ylethyl)(1H-indazol-6-yl)]-2- | B |
| morpholin-4-ylpyrimidin-4-yl}amine | |
| (4-chlorophenyl)[6-(2-methyl-1-oxetan-3-ylbenzimidazol-6-yl)-2-morpholin- | A |
| 4-ylpyrimidin-4-yl]amine | |
| (4-chlorophenyl)[6-(3-ethyl-1-methyl(1H-indazol-6-yl))-2-morpholin-4- | C |
| ylpyrimidin-4-yl]amine | |
| (4-imidazol-2-ylphenyl){6-[1-methyl-3-(2-morpholin-4-ylethyl)(1H-indazol- | C |
| 6-yl)]-2-morpholin-4-ylpyrimidin-4-yl}amine | |
| {6-[1-methyl-3-(2-morpholin-4-ylethyl)(1H-indazol-6-yl)]-2-morpholin-4- | B |
| ylpyrimidin-4-yl}(4-(1,3-oxazol-2-yl)phenyl)amine | |
| (4-chlorophenyl)[6-(2-methyl-1-oxetan-3-ylbenzimidazol-5-yl)-2-morpholin- | A |
| 4-ylpyrimidin-4-yl]amine | |
| [6-(1-methyl(1H-indazol-6-yl))-2-morpholin-4-ylpyrimidin-4-yl](4-(1,2,4- | B |
| triazolyl)phenyl)amine | |
| [6-(1,3-dimethyl(1H-indazol-6-yl))-2-morpholin-4-ylpyrimidin-4-yl]{4-[5- | A |
| (methylethyl)(4H-1,2,4-triazol-3-yl)]phenyl}amine | |
| {6-[1-methyl-3-(2-morpholin-4-ylethyl)(1H-indazol-6-yl)]-2-morpholin-4- | A |
| ylpyrimidin-4-yl}[4-(1-methylpyrrol-2-yl)phenyl]amine | |
| {6-[1-methyl-3-(2-morpholin-4-ylethyl)(1H-indazol-6-yl)]-2-morpholin-4- | A |
| ylpyrimidin-4-yl}(4-(1,3-oxazol-4-yl)phenyl)amine | |
| (4-chlorophenyl){6-[1-methyl-3-(2-piperazinylethyl)(1H-indazol-6-yl)]-2- | C |
| morpholin-4-ylpyrimidin-4-yl}amine | |
| [4-(1-methylimidazol-2-yl)phenyl]{6-[1-methyl-3-(2-morpholin-4- | B |
| ylethyl)(1H-indazol-6-yl)]-2-morpholin-4-ylpyrimidin-4-yl}amine | |
| (4-imidazol-4-ylphenyl){6-[1-methyl-3-(2-morpholin-4-ylethyl)(1H-indazol- | C |
| 6-yl)]-2-morpholin-4-ylpyrimidin-4-yl}amine | |
| (4-(4H-1,2,4-triazol-3-yl)phenyl)[6-(1,3-dimethylpyrazolo[4,5-b]pyridin-6- | A |
| yl)-2-morpholin-4-ylpyrimidin-4-yl]amine | |
| (1-ethylindol-6-yl)[6-(1-methyl(1H-indazol-6-yl))-2-morpholin-4-ylpyrimidin- | C |
| 4-yl]amine | |
| (4-chlorophenyl)(6-{1-methyl-3-[2-(4-methylpiperazinyl)ethyl](1H-indazol-6- | B |
| yl)}-2-morpholin-4-ylpyrimidin-4-yl)amine | |
| [6-(1-methyl(1H-indazol-6-yl))-2-morpholin-4-ylpyrimidin-4-yl](4-(1,2,3- | A |
| triazol-2-yl)phenyl)amine | |
| [6-(1,3-dimethyl(1H-indazol-6-yl))-2-morpholin-4-ylpyrimidin-4-yl](4-(1,2,3- | A |
| triazol-2-yl)phenyl)amine | |
| [6-(1,3-dimethyl(1H-indazol-6-yl))-2-morpholin-4-ylpyrimidin-4-yl](1- | C |
| ethylindol-6-yl)amine | |
| {6-[1-(cyclopropylmethyl)-2-methylbenzimidazol-6-yl]-2-morpholin-4- | A |
| ylpyrimidin-4-yl}(1-methylindol-6-yl)amine | |
| [6-(1-methyl(1H-indazol-6-yl))-2-morpholin-4-ylpyrimidin-4-yl]{4-[5- | B |
| (trifluoromethyl)imidazol-2-yl]phenyl}amine | |
| [4-(3-methyl(1,2,4-triazol-4-yl))phenyl][6-(1-methyl(1H-indazol-6-yl))-2- | B |
| morpholin-4-ylpyrimidin-4-yl]amine | |
| [6-(1,3-dimethyl(1H-indazol-6-yl))-2-morpholin-4-ylpyrimidin-4-yl]{4-[5- | B |
| (trifluoromethyl)imidazol-2-yl]phenyl}amine | |
| {6-[1-methyl-3-(2-morpholin-4-ylethyl)(1H-indazol-6-yl)]-2-morpholin-4- | A |
| ylpyrimidin-4-yl}(4-pyrazol-5-ylphenyl)amine | |
| (1-cyclopropylindol-6-yl) {6-[1-methyl-3-(2-morpholin-4-ylethyl)(1H-indazol- | C |
| 6-yl)]-2-morpholin-4-ylpyrimidin-4-yl}amine | |
| 5-{6-[(4-chlorophenyl)amino]-2-morpholin-4-ylpyrimidin-4-yl}-1,3-dimethyl- | A |
| 3-hydrobenzimidazol-2-one | |
| (6-(1H-indazol-4-yl)-2-morpholin-4-ylpyrimidin-4-yl)(4-chlorophenyl)amine | B |
| {6-[3-(3-aminopropyl)-1-methyl(1H-indazol-6-yl)]-2-morpholin-4- | A |
| ylpyrimidin-4-yl}(1-methylindol-6-yl)amine | |
| 5-{6-[(4-chlorophenyl)amino]-2-morpholin-4-ylpyrimidin-4-yl}-3-methyl-1- | B |
| (2-morpholin-4-ylethyl)-3-hydrobenzimidazol-2-one | |
| (6-benzimidazol-4-yl-2-morpholin-4-ylpyrimidin-4-yl)(4-chlorophenyl)amine | B |
| [4-(4-fluoropyrazol-5-yl)phenyl][6-(1-methyl(1H-indazol-6-yl))-2-morpholin- | C |
| 4-ylpyrimidin-4-yl]amine | |
| [6-(1,3-dimethyl(1H-indazol-6-yl))-2-morpholin-4-ylpyrimidin-4-yl][4-(4- | B |
| fluoropyrazol-5-yl)phenyl]amine | |
| {6-[1-methyl-3-(2-morpholin-4-ylethyl)(1H-indazol-6-yl)]-2-morpholin-4- | B |
| ylpyrimidin-4-yl}(1,2,3-trimethylindol-6-yl)amine | |
| 2-[3-(1-methyl-6-{6-[(1-methylindol-6-yl)amino]-2-morpholin-4-ylpyrimidin- | A |
| 4-yl}-1H-indazol-3-yl)propyl]benzo[c]azoline-1,3-dione | |
| {6-[1-methyl-3-(2-morpholin-4-ylethyl)(1H-indazol-6-yl)]-2-morpholin-4- | A |
| ylpyrimidin-4-yl}(4-(1,2,3-triazol-2-yl)phenyl)amine | |
| (4-chlorophenyl)[6-(3-ethyl-1-methylpyrazolo[4,5-b]pyridin-6-yl)-2- | C |
| morpholin-4-ylpyrimidin-4-yl]amine | |
| [6-(1,3-dimethyl(1H-indazol-6-yl))-2-morpholin-4-ylpyrimidin-4-yl](1,2,3- | C |
| trimethylindol-6-yl)amine | |
| [4-(1-methyl(1,2,3-triazol-4-yl))phenyl][6-(1-methyl(1H-indazol-6-yl))-2- | C |
| morpholin-4-ylpyrimidin-4-yl]amine | |
| [6-(1,3-dimethyl(1H-indazol-6-yl))-2-morpholin-4-ylpyrimidin-4-yl](4-{1-[(4- | C |
| methoxyphenyl)methyl]pyrazol-4-yl}phenyl)amine | |
| [4-(5-cyclopropyl(4H-1,2,4-triazol-3-yl))phenyl]{6-[1-methyl-3-(2-morpholin- | A |
| 4-ylethyl)(1H-indazol-6-yl)]-2-morpholin-4-ylpyrimidin-4-yl}amine | |
| 2-[2-(1-methyl-6-{6-[(1-methylindol-6-yl)amino]-2-morpholin-4-ylpyrimidin- | B |
| 4-yl}-1H-indazol-3-yl)ethyl]benzo[c]azoline-1,3-dione | |
| dimethyl[3-(1-methyl-6-{6-[(1-methylindol-6-yl)amino]-2-morpholin-4- | A |
| ylpyrimidin-4-yl} (1H-indazol-3-yl))propyl]amine | |
| (1-methylindol-6-yl) {6-[1-methyl-3-(3-pyrrolidinylpropyl)(1H-indazol-6-yl)]- | B |
| 2-morpholin-4-ylpyrimidin-4-yl}amine | |
| [6-(1,3-dimethylpyrazolo[4,5-b]pyridin-6-yl)-2-morpholin-4-ylpyrimidin-4- | A |
| yl](4-(1,2,3-triazol-2-yl)phenyl)amine | |
| (1,3-dimethylindol-6-yl){6-[1-methyl-3-(2-morpholin-4-ylethyl)(1H-indazol- | B |
| 6-yl)]-2-morpholin-4-ylpyrimidin-4-yl}amine | |
| (1,2-dimethylindol-6-yl){6-[1-methyl-3-(2-morpholin-4-ylethyl)(1H-indazol- | B |
| 6-yl)]-2-morpholin-4-ylpyrimidin-4-yl}amine | |
| [6-(1,3-dimethyl(1H-indazol-6-yl))-2-morpholin-4-ylpyrimidin-4-yl](1,2- | B |
| dimethylindol-6-yl)amine | |
| {6-[3-(2-aminoethyl)-1-methyl(1H-indazol-6-yl)]-2-morpholin-4-ylpyrimidin- | A |
| 4-yl}(1-methylindol-6-yl)amine | |
| [6-(1,3-dimethyl(1H-indazol-6-yl))-2-morpholin-4-ylpyrimidin-4-yl](4-chloro- | C |
| 1-methylindol-6-yl)amine | |
| [6-(1-methyl(1H-indazol-6-yl))-2-morpholin-4-ylpyrimidin-4-yl][4-(3- | A |
| methylpyrazolyl)phenyl]amine | |
| [6-(1,3-dimethyl(1H-indazol-6-yl))-2-morpholin-4-ylpyrimidin-4-yl][4-(3- | A |
| methylpyrazolyl)phenyl]amine | |
| [6-(1,3-dimethyl(1H-indazol-6-yl))-2-morpholin-4-ylpyrimidin-4-yl](4- | A |
| pyrazolylphenyl)amine | |
| [6-(1-methyl(1H-indazol-6-yl))-2-morpholin-4-ylpyrimidin-4-yl](1,2,3- | C |
| trimethylindol-6-yl)amine | |
| [6-(1-methyl(1H-indazol-6-yl))-2-morpholin-4-ylpyrimidin-4-yl][4-(4- | C |
| methylpyrazolyl)phenyl]amine | |
| [6-(1,3-dimethyl(1H-indazol-6-yl))-2-morpholin-4-ylpyrimidin-4-yl][4-(4- | C |
| methylpyrazolyl)phenyl]amine | |
| {6-[1-methyl-3-(2-morpholin-4-ylethyl)(1H-indazol-6-yl)]-2-morpholin-4- | A |
| ylpyrimidin-4-yl}[4-(3-methylpyrazolyl)phenyl]amine | |
| [6-(1-methyl(1H-indazol-6-yl))-2-morpholin-4-ylpyrimidin-4-yl](4- | A |
| pyrazolylphenyl)amine | |
| {6-[1-methyl-3-(2-morpholin-4-ylethyl)(1H-indazol-6-yl)]-2-morpholin-4- | A |
| ylpyrimidin-4-yl}(4-pyrazolylphenyl)amine | |
| {6-[1-(cyclopropylmethyl)-2-(2-morpholin-4-ylethyl)benzimidazol-6-yl]-2- | A |
| morpholin-4-ylpyrimidin-4-yl}(1-methylindol-6-yl)amine | |
| [4-(4-methyl(1,2,3-triazol-2-yl))phenyl][6-(1-methyl(1H-indazol-6-yl))-2- | A |
| morpholin-4-ylpyrimidin-4-yl]amine | |
| [6-(1,3-dimethyl(1H-indazol-6-yl))-2-morpholin-4-ylpyrimidin-4-yl][4-(4- | A |
| methyl(1,2,3-triazol-2-yl))phenyl]amine | |
| [4-(4-methyl(1,2,3-triazol-2-yl))phenyl]{6-[1-methyl-3-(2-morpholin-4- | A |
| ylethyl)(1H-indazol-6-yl)]-2-morpholin-4-ylpyrimidin-4-yl}amine | |
| 4-(4-{[6-(1-methyl(1H-indazol-6-yl))-2-morpholin-4-ylpyrimidin-4- | C |
| yl]amino}phenyl)-1,2,4-triazolin-5-one | |
| [4-(4,5-dimethyl(1,2,3-triazol-2-yl))phenyl][6-(1,3-dimethyl(1H-indazol-6- | C |
| yl))-2-morpholin-4-ylpyrimidin-4-yl]amine | |
| (4-chlorophenyl){6-[5-fluoro-1-methyl-3-(2-morpholin-4-ylethyl)(1H-indazol- | B |
| 6-yl)]-2-morpholin-4-ylpyrimidin-4-yl}amine | |

### MPS IIIA Disease Mouse Model

MPS IIIA mice (Sgsh−/−) are obtained from Jackson Laboratory (B6.Cg-Sgsh) and were housed in Association for Assessment and Accreditation of Laboratory Animal Care (AAALAC)-approved vivaria in the School of Medicine, University of California, San Diego, following standards and procedures approved by the local Institutional Animal Care and Use Committee for the ethical use of animals in experiments. Canine samples are provided by P. Dickson (University of California, Harbor). MPS MA mice (see Bhaumik et al. Glycobiol 1999, 9, 1389) are injected with an amount (e.g., about 100 mg/kg/day) of an HS biosynthesis inhibitor. Samples of mouse urine are incubated for one hour at 37° C. with two volumes of 0.1% cetylpyridinium chloride in 0.054 M sodium citrate (pH 4.8). Samples are centrifuged for 10 minutes at 3000 rpm and pellets are resuspended in 150 μL 2 M LiCl. Following addition of 800 μL absolute ethanol, samples are incubated at −20° C. for one hour and then centrifuged for 10 minutes at 3000 rpm. Pellets are resuspended in 200 μL of water, lyophilized, and then resuspended in 20 μL water. Purified glycosaminoglycan samples (0.2 μmol creatinine equivalents) are analyzed on 40-50% linear gradient polyacrylamide gels. Samples are compared to results for a control MPS IIIA mouse (or population thereof) that was not exposed to an HS biosynthesis inhibitor.

### MPS IIIB Disease Mouse Model

Samples from MPS IIIB mice are provided by E. Neufeld (University of California, Los Angeles). Human urine samples without personal identifying information are obtained from donors from an MPS patient advocacy group with informed consent.

### Substrate Reduction Therapy

The non-specific inhibitor of sulfation, sodium chlorate, is used for validation or SRT. 30-60 mM sodium chlorate inhibits the synthesis of PAPs, the sulfate donor used in all cellular sulfation reactions including heparan sulfate biosynthesis. Cells grown in the presence of sodium chlorate produce heparan sulfate with reduced sulfation. In certain instances, in vitro MPS models are based on measuring the accumulation of GAG fragments in cultured primary human fibroblast from MPS patients. In some instances, the GAGs that accumulate in MPS patients are much smaller than normal tissue GAGs and they lack a core protein on their reducing termini. Based on these features the in vitro MPS model is based on a method of tagging reducing ends of the GAGs with a detectable label and analyzing (i.e., detecting and/or measuring) the detectable labels using a device suitable for detecting the label (e.g., an HPLC with a fluorimeter).

Other objects, features and advantages of the compounds, methods and compositions described herein will become apparent from the following description. It should be understood, however, that the description and the specific examples, while indicating specific embodiments, are given by way of illustration only, since various changes and modifications within the spirit and scope of the present description will become apparent from this detailed description.

All publications including patents, patent applications and published patent applications cited herein are hereby incorporated by reference for all purposes.

1. A compound of Formula I: 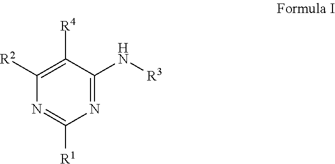
wherein:
R¹ is heterocycloalkyl optionally substituted with 1, 2, 3, or 4 alkyl;
R² is a 9-membered bicyclic ring comprising 1, 2, 3, or 4 nitrogen atoms where one or both of the rings is aromatic, where a carbon atom in R² is the point of attachment to the pyrimidinyl in Formula I, and where R² is optionally substituted with 1 oxo and additionally optionally substituted with 1, 2, or 3 R^{2a} groups;
each R^{2a} is independently selected from cyano, nitro, halo, hydroxy, alkyl, alkenyl, carboxy, alkoxycarbonyl, alkylcarbonyl, alkylsulfinyl, alkylsulfonyl, alkylsulfonyloxyalkyl, alkoxyalkyl, alkoxyalkenyl, aminoalkyl, alkylaminoalkyl, dialkylaminoalkyl, hydroxyalkyl, cycloalkyl, cycloalkylalkyl, heterocycloalkyl, heterocycloalkylalkyl, 1,3-dioxo-isoindolinylalkyl, —NR^{2b}R^{2c}, and —OR^{2d}; where each heterocycloalkyl, either alone or as part of another group, is optionally substituted with 1, 2, 3, or 4 groups independently selected from alkyl, hydroxy, alkylcarbonyl, and alkoxycarbonyl;
R^{2b} is hydrogen or alkyl;
R^{2c} is hydrogen, alkyl, aminoalkyl, alkylaminoalkyl, dialkylaminoalkyl, alkoxyalkyl, hydroxyalkyl, alkylcarbonyl, cycloalkylcarbonyl, alkyl sulfonyl, alkoxycarbonyl, cycloalkylalkyl, or heterocycloalkylalkyl; where the heterocycloalkyl in heterocycloalkylalkyl is optionally substituted with 1 or 2 groups independently selected from alkyl, hydroxy, alkylcarbonyl, and alkoxycarbonyl;
R^{2d} is alkyl, haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl; where each heterocycloalkyl, either alone or as part of another group, is optionally substituted with 1 or 2 groups independently selected from alkyl, hydroxy, alkylcarbonyl, and alkoxycarbonyl;
R³ is phenyl or heteroaryl each of which is optionally substituted with 1, 2, or 3 R^{3a} groups;
each R^{3a} is independently selected from —C(═NH)NHOH, cyano, nitro, halo, hydroxy, alkyl, alkoxycarbonyl, alkoxyalkyl, cycloalkyl, cycloalkylalkyl, heterocycloalkyl, heterocycloalkylalkyl, alkylcarbonyl, alkylsulfinyl, alkyl sulfonyl, —NR^{3b}R^{3c}, —C(O)NR^{3b}R^{3c}, —S(O)₂NR^{3b}R^{3c}, and heteroaryl optionally substituted with 1, 2, or 3 R⁵ groups;
where the heterocycloalkyl either alone or as part of heterocycloalkylalkyl is optionally substituted with 1 or two alkyl groups;
R^{3b} is hydrogen or alkyl;
R^{3c} is hydrogen, alkyl, haloalkyl, aminoalkyl, alkylaminoalkyl, dialkylaminoalkyl, alkoxyalkyl, alkylcarbonyl, alkyl sulfonyl, alkoxycarbonyl, cycloalkylalkyl, heterocycloalkyl, heterocycloalkylalkyl, or cycloalkyl optionally substituted with 1 or 2 alkyl; or R^{3b} and R^{3c} together with the nitrogen to which they are attached form heterocycloalkyl;
R^{3d} is alkyl, haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl;
R⁴ is hydrogen, methyl, halo, or —CN; and
each R⁵ is independently halo, hydroxy, alkoxycarbonyl, alkyl, haloalkyl, cycloalkyl, cycloalkylalkyl, heterocycloalkyl, heterocycloalkylalkyl, phenyl, or phenylmethyl which is optionally substituted with 1 or 2 alkoxy;
optionally as a single stereoisomer or mixture of stereoisomers thereof and additionally optionally as a pharmaceutically acceptable salt thereof;
provided that:
a) the compound is not N-(6-(1H-imidazo[4,5-b]pyridin-6-yl)-2-morpholinopyrimidin-4-yl)quinolin-3-amine or N-(6-(1H-pyrazolo[3,4-b]pyridin-5-yl)-2-morpholinopyrimidin-4-yl)quinolin-3-amine;
b) when R³ is pyrazolyl substituted with one R^{3a}, then R^{3a} is not cyclopropyl; and
c) when R³ is phenyl substituted with one R^{3a}, then the one R^{3a} is not 3-7-membered cycloalkyl ring.

2. The compound of claim 1, wherein:
R¹ is dihydro-2H-_{pyran-}4-yl, tetrahydro-2H-pyran-4-yl, or morpholin-4-yl, each of which is optionally substituted with 1 or 2 alkyl;
R³ is phenyl substituted with 1, 2, or 3 R^{3a} groups independently selected from —C(═NH)NHOH, cyano, nitro, halo, alkyl, alkoxycarbonyl, alkoxyalkyl, cycloalkyl, cycloalkylalkyl, heterocycloalkyl, heterocycloalkylalkyl, alkylcarbonyl, alkylsulfinyl, alkylsulfonyl, —OR^{3d}, —NR^{3b}R^{3c}, —C(O)NR^{3b}R^{3c}, —S(O)₂NR^{3b}R^{3c}, and heteroaryl optionally substituted with 1, 2, or 3 R⁵ groups; where the heterocycloalkyl either alone or as part of heterocycloalkylalkyl is optionally substituted with 1 alkyl; provided that R³ is not 3-amino-phenyl or 3,4-dimethylphenyl; or
R³ is 6-10 membered heteroaryl each of which is substituted with 1, 2, or 3 R^{3a} groups independently selected from —C(═NH)NHOH, cyano, nitro, halo, hydroxy, alkyl, alkoxycarbonyl, alkoxyalkyl, cycloalkyl, cycloalkylalkyl, heterocycloalkyl, heterocycloalkylalkyl, alkylcarbonyl, alkylsulfinyl, alkyl sulfonyl, —OR^{3d}, —NR^{3b}R^{3c}, —C(O)NR^{3b}R^{3c}, —S(O)₂NR^{3b}R^{3c}, and heteroaryl optionally substituted with 1, 2, or 3 R⁵ groups; provided that R³ is not 2-oxo-1H-benzo[d]imidazolyl, 1-ethyl-2-methyl-1H-benzo[d]imidazolyl, or 1-acetyl-indolinyl;
R^{3b} is hydrogen or alkyl;
R^{3c} is hydrogen, alkyl, haloalkyl, aminoalkyl, alkylaminoalkyl, dialkylaminoalkyl, alkoxyalkyl, alkylcarbonyl, alkyl sulfonyl, alkoxycarbonyl, cycloalkylalkyl, heterocycloalkyl, heterocycloalkylalkyl, or cycloalkyl optionally substituted with 1 or 2 alkyl; or R^{3b} and R^{3c} together with the nitrogen to which they are attached form heterocycloalkyl;
R^{3d} is haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl;
R⁴ is hydrogen, methyl, or halo; and
each R⁵ is independently halo, hydroxy, alkoxycarbonyl, alkyl, haloalkyl, cycloalkyl, cycloalkylalkyl, heterocycloalkyl, heterocycloalkylalkyl, phenyl, or phenylmethyl which is optionally substituted with 1 or 2 alkoxy;
optionally as a single stereoisomer or mixture of stereoisomers thereof and additionally optionally as a pharmaceutically acceptable salt thereof.

3. The compound of claim 1, wherein:
R¹ is morpholin-4-yl;
R² is a 9-membered bicyclic ring comprising 1, 2, or 3 nitrogen atoms where one or both of the rings is aromatic, where a carbon atom in R² is the point of attachment to the pyrimidinyl in Formula I, and where R² is optionally substituted with 1 oxo and additionally substituted with 1, 2, or 3 R^{2a} groups;
each R^{2a} is independently selected from halo, alkyl, alkoxyalkyl, aminoalkyl, alkylaminoalkyl, dialkylaminoalkyl, hydroxyalkyl, cycloalkyl, cycloalkylalkyl, heterocycloalkyl, heterocycloalkylalkyl, 1,3-dioxo-isoindolinylalkyl, and —NR^{2b}R^{2c}; where each heterocycloalkyl, either alone or as part of another group, is optionally substituted with alkyl;
R^{2b} is hydrogen or alkyl;
R^{2c} is alkyl, aminoalkyl, alkylaminoalkyl, dialkylaminoalkyl, alkoxyalkyl, or heterocycloalkylalkyl;
R³ is phenyl or heteroaryl each of which is optionally substituted with 1, 2, or 3 R^{3a} groups;
each R^{3a} is independently selected from cyano, halo, alkyl, alkoxycarbonyl, cycloalkyl, alkylcarbonyl, alkylsulfinyl, alkylsulfonyl, —OR^{3d}, —NR^{3b}R^{3c}, —C(O)NR^{3b}R^{3c}, —S(O)₂NR^{3b}R^{3c}, and heteroaryl optionally substituted with R⁵;
R^{3b} is hydrogen or alkyl;
R^{3c} is hydrogen, alkyl, alkylcarbonyl, alkylsulfonyl, cycloalkylalkyl, or cycloalkyl optionally substituted with alkyl;
R^{3d} is alkyl;
R⁴ is hydrogen, or halo; and
each R⁵ is independently halo, alkyl, haloalkyl, cycloalkyl, or phenylmethyl which is optionally substituted with alkoxy;
optionally as a single stereoisomer or mixture of stereoisomers thereof and additionally optionally as a pharmaceutically acceptable salt thereof.

4. The compound of any of claim 1, wherein R¹ is morpholin-4-yl.

5. The compound of any of claim 1, wherein R² is indazolyl or pyrazolopyridinyl, each of which is optionally substituted on any atom of the ring with 1, 2, or 3 R^{2a} groups.

6. The compound of claim 1, wherein R² is benzimidazolyl or imidazopyridinyl, each of which is optionally substituted on any atom of the ring with 1, 2, or 3 R^{2a} groups.

7. The compound of claim 1, wherein R² is 2-oxo-1H-benzo[d]imidazolyl optionally substituted on any atom of the ring with 1, 2, or 3 R^{2a} groups.

8. The compound of claim 1, wherein R² is indazolyl or benzimidazolyl, each of which is optionally substituted on any atom of the ring with 1, 2, or 3 R^{2a} groups.

9. The compound of claim 1, wherein R² is substituted with 1, 2, or 3 R^{2a} groups independently selected from alkyl, hydroxyalkyl, alkoxyalkyl, halo, cycloalkyl, cycloalkylalkyl, heterocycloalkyl, heterocycloalkylalkyl optionally substituted with one alkyl, aminoalkyl, alkylaminoalkyl, dialkylaminoalkyl, 1,3-dioxo-isoindolinylalkyl, and —NR^{2b}R^{2c}.

10. The compound of claim 1, wherein R^{2a} is alkyl or heterocycloalkylalkyl.

11. The compound of claim 1, wherein R^{2a} is C₁₋₃ alkyl or heterocycloalkyl(C₁₋₃)alkyl, where the heterocycloalkyl group is morpholinyl, piperzinyl, or pyrrolodinyl.

12. The compound of claim 1, wherein R³ is phenyl substituted with one or two R^{3a} groups.

13. The compound of claim 1, wherein R³ is phenyl substituted with halo.

14. The compound of claim 1, wherein R³ is phenyl substituted with chloro.

15. The compound of claim 1, wherein R³ is phenyl substituted with —C(O)NR^{3b}R^{3c}.

16. The compound of any of claim 1, wherein R³ is phenyl substituted with a 5-membered heteroaryl optionally substituted with one R⁵.

17. The compound of claim 16, wherein R³ is phenyl substituted with R^{3a} where R^{3a} is triazolyl, oxazolyl, imidazolyl, oxadiazolyl, pyrazolyl, or pyrrolyl, each of which is optionally substituted with one R⁵.

18. The compound of claim 17, wherein R³ is phenyl substituted with R^{3a} where R^{3a} is triazolyl, oxazolyl, imidazolyl, oxadiazolyl, pyrazolyl, or pyrrolyl, each of which is optionally substituted with alkyl, halo, haloalkyl, cycloalkyl, or phenylmethyl, where the phenylmethyl is optionally substituted with alkoxy.

19. The compound of claim 1, wherein R³ is a 6-10 membered heteroaryl substituted with 1, 2, or 3 R^{3a} groups.

20. The compound of claim 19, wherein R³ is pyridyl substituted with 1, 2, or 3 R^{3a} groups.

21. The compound of claim 19, wherein R³ is a 9-membered heteroaryl with 1, 2, or 3 nitrogen atoms, optionally substituted with 1, 2, or 3 R^{3a} groups.

22. The compound of claim 21, where R³ is indolyl, benzoisoxazolyl, indazolyl, benzotriazolyl, benzoxazolyl, or benzimidazolyl, each of which is optionally substituted with 1, 2, or 3 alkyl groups.

23. The compound of claim 1, wherein:
R¹ is morpholin-4-yl;
R² is indazolyl or benzimidazolyl, either of which is substituted with at least one R^{2a} independently selected from alkyl, hydroxyalkyl, alkoxyalkyl, halo, cycloalkyl, cycloalkylalkyl, heterocycloalkyl, heterocycloalkylalkyl optionally substituted with one alkyl, aminoalkyl, alkylaminoalkyl, dialkylaminoalkyl, 1,3-dioxo-isoindolinylalkyl, and —NR^{2b}R^{2c};
R³ is phenyl substituted with at least one R^{3a} independently selected from halo, —C(O)NR^{3b}R^{3c}, and a 5-membered heteroaryl optionally substituted with one R⁵; or
R³ is pyridinyl, indolyl, benzoisoxazolyl, indazolyl, benzotriazolyl, benzoxazolyl, or benzimidazolyl, each of which is optionally substituted with 1, 2, or 3 alkyl groups.

24. The compound of claim 1 according to Formula I(a): 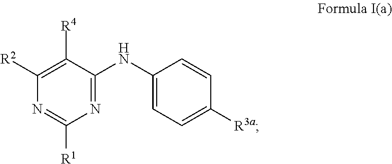
optionally as a single stereoisomer or mixture of stereoisomers thereof and additionally optionally as a pharmaceutically acceptable salt thereof.

25. The compound of claim 24, wherein:
R¹ is morpholin-4-yl;
R² is indazolyl or benzimidazolyl, either of which is substituted with at least one R^{2a} independently selected from alkyl, hydroxyalkyl, alkoxyalkyl, halo, cycloalkyl, cycloalkylalkyl, heterocycloalkyl, heterocycloalkylalkyl optionally substituted with 1 alkyl, aminoalkyl, alkylaminoalkyl, dialkylaminoalkyl, 1,3-dioxo-isoindolinylalkyl, and —NR^{2b}R^{2c};
R³ is phenyl substituted with at least one R^{3a} independently selected from halo, —C(O)NR^{3b}R^{3c}, and a 5-membered heteroaryl optionally substituted with one R⁵; or
R³ is pyridinyl, indolyl, benzoisoxazolyl, indazolyl, benzotriazolyl, benzoxazolyl, or benzimidazolyl, each of which is optionally substituted with 1, 2, or 3 alkyl groups.

26. A compound selected from Table 1; optionally as a single stereoisomer or mixture of stereoisomers thereof and additionally optionally as a pharmaceutically acceptable salt thereof.

27. A pharmaceutical composition comprising a compound of claim 1 and a pharmaceutically acceptable excipient.

28. A method of treating a disease or disorder comprising administering to a subject in need thereof a therapeutically effective amount of a compound according to claim 1.

29. A method of treating a disease or disorder mediated by inhibition of heparan sulfate biosynthesis comprising administering to a subject in need thereof a therapeutically effective amount of a compound according to claim 1.

30. A method of treatment comprising administering to a subject having a disease or disorder mediated by inhibition of heparan sulfate biosynthesis a compound according to claim 1, wherein the compound or composition is administered in an amount effective to treat the disease or disorder.

31. The method of claim 28, wherein the method further comprises identifying the subject in need thereof.

32. The method of claim 29, wherein the method further comprises identifying the subject having a disease or disorder mediated by inhibition of heparan sulfate biosynthesis.

33. The method of claim 28, wherein the disease is an amyloid disease, an autoimmune disorder, a CNS disorder, MPS I, MPS II, MPS IIIA, MPS IIIB, MPS IIIC, or MPS IIID.

34. The method of claim 28, wherein the disease is Alzheimer's disease, Parkinson's disease, type 2 diabetes, chronic hemodialysis-related amyloid, MPS I, MPS II, MPS IIIA, MPS IIIB, MPS IIIC, multiple sclerosis, rheumatoid arthritis, juvenile chronic arthritis, psoriasis, psoriatic arthritis, or Crohn's disease.

35. The method of claim 34, wherein the disease is MPS I, II, IIIA, IIIB, or IIIC.

36. The method of claim 28, further comprising administering enzyme replacement therapy to the subject.

37. A method of making a compound according to claim 1, comprising
a) treating an intermediate of formula 102: 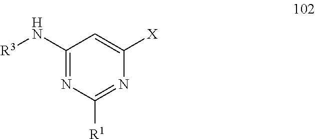
where X is halo, or a salt thereof; with an intermediate of formula R²B(OR)₂ in the presence of a catalyst and a base to yield a compound of Formula I, wherein each R is independently hydrogen or alkyl or together with the atoms to which they are attached form a carbocyclic ring; or
b) treating an intermediate of formula 101: 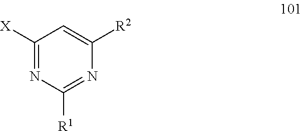
where X is halo, or a salt thereof; with an intermediate of formula R³NH₂ in the presence of a catalyst and a base to yield a compound of Formula I; and
c) optionally separating individual isomers.

38. A compound of Formula II: 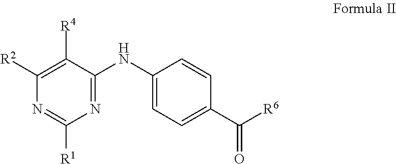
wherein:
R¹ is heterocycloalkyl optionally substituted with 1, 2, 3, or 4 alkyl;
R² is a 9-membered bicyclic ring comprising 1, 2, 3, or 4 nitrogen atoms where one or both of the rings is aromatic, where a carbon atom in R² is the point of attachment to the pyrimidinyl in Formula II, and where R² is optionally substituted with 1 oxo and additionally optionally substituted with 1, 2, or 3 R^{e}a groups;
each R^{2a} a is independently selected from cyano, nitro, halo, hydroxy, alkyl, alkenyl, carboxy, alkoxycarbonyl, alkylcarbonyl, alkylsulfinyl, alkylsulfonyl, alkylsulfonyloxyalkyl, alkoxyalkyl, alkoxyalkenyl, aminoalkyl, alkylaminoalkyl, dialkylaminoalkyl, hydroxyalkyl, cycloalkyl, cycloalkylalkyl, heterocycloalkyl, heterocycloalkylalkyl, 1,3-dioxo-isoindolinylalkyl, —NR^{2b}R^{2c}, and —OR^{2d}; where each heterocycloalkyl, either alone or as part of another group, is optionally substituted with 1, 2, 3, or 4 groups independently selected from alkyl, hydroxy, alkylcarbonyl, and alkoxycarbonyl;
R^{2b} is hydrogen or alkyl;
R^{2c} is hydrogen, alkyl, aminoalkyl, alkylaminoalkyl, dialkylaminoalkyl, alkoxyalkyl, hydroxyalkyl, alkylcarbonyl, cycloalkylcarbonyl, alkyl sulfonyl, alkoxycarbonyl, cycloalkylalkyl, or heterocycloalkylalkyl; where the heterocycloalkyl in heterocycloalkylalkyl is optionally substituted with 1 or 2 groups independently selected from alkyl, hydroxy, alkylcarbonyl, and alkoxycarbonyl;
R^{2d} is alkyl, haloalkyl, hydroxyalkyl, alkoxyalkyl, aminoalkyl, alkylaminoalkyl, dialkylaminoalkyl, cycloalkyl, cycloalkylalkyl, heterocycloalkyl, or heterocycloalkylalkyl; where each heterocycloalkyl, either alone or as part of another group, is optionally substituted with 1 or 2 groups independently selected from alkyl, hydroxy, alkylcarbonyl, and alkoxycarbonyl;
R⁴ is hydrogen, methyl, halo, or —CN; and
R⁶ is halo, hydroxy, or alkoxy;
optionally as a single stereoisomer or mixture of stereoisomers thereof and additionally optionally as a pharmaceutically acceptable salt thereof.
